(19) United States
(12) Patent Application Publication
(10) Pub. No.: US 2026/0078104 A1
(43) Pub. Date: Mar. 19, 2026

(54) ORGANIC LIGHT-EMITTING COMPOUND AND ORGANIC ELECTROLUMINESCENT DEVICE USING THE SAME

(71) Applicant: SOLUS ADVANCED MATERIALS CO., LTD., Iksan-si (KR)

(72) Inventors: Woo Jae PARK, Yongin-si (KR); Min Sik Eum, Yongin-si (KR); Jae Yi Sim, Yongin-si (KR)

(73) Assignee: SOLUS ADVANCED MATERIALS CO., LTD., Iksan-si (KR)

(21) Appl. No.: 19/395,695

(22) Filed: Nov. 20, 2025

Related U.S. Application Data
(62) Division of application No. 16/632,009.

(30) Foreign Application Priority Data
Jul. 20, 2017 (KR) 10-2017-0092063

(51) Int. Cl. C07D 401/04; C07D 401/10; H10K 50/15; H10K 50/16; H10K 50/17; H10K 85/60

(52) U.S. Cl. CPC C07D 401/04; C07D 401/10; H10K 85/654; H10K 50/15; H10K 50/166; H10K 50/17; H10K 50/171

(57) ABSTRACT

The present disclosure relates to a novel organic compound and an organic EL device including the organic compound. The compound according to the present disclosure may be used in an organic layer of an organic EL device, more specifically, in a light emitting layer, a light emitting auxiliary layer, an electron transport auxiliary layer, or an electron transporting layer and may improve driving voltage, luminous efficiency, and lifespan characteristics of the organic EL device.

### CROSS REFERENCE TO RELATED APPLICATIONS

This application is a Divisional application of U.S. application Ser. No. 16/632,009 filed Jan. 17, 2020, which is a National Stage of International Application No. PCT/KR2018/007482 filed Jul. 2, 2018, claiming priority based on Korean Patent Application No. 10-2017-0092063 filed Jul. 20, 2017.

### TECHNICAL FIELD

The present disclosure relates to a novel light-emitting organic compound and an organic electroluminescent device using the same, and more particularly, to a compound having excellent electron transporting ability and light emitting performance and an organic electroluminescence device improved in terms of luminous efficiency, driving voltage, lifespan and the like by including the compound in one or more organic layers.

### DISCUSSION OF RELATED ART

Starting from Bernanose's observation of light emission from organic thin films in the 1950s, the study on organic electroluminescent devices (hereinafter, “EL devices”) led to blue electroluminescence using anthracene monocrystals in 1965, and Tang suggested in 1987 an organic EL device in a stack structure which may be divided into functional layers of hole layers and light emitting layers. Then, in order to develop highly efficient, long lifespan organic EL devices, organic layers each having distinctive characteristics have been introduced in the EL devices, which led to the development of specialized materials used therein.

In organic EL devices, upon application of voltage between two electrodes, holes are injected from an anode into an organic layer and electrons are injected from a cathode into the organic layer. Injected holes and electrons meet each other to form excitons, and light emission occurs when the excitons fall to a ground state. In this case, materials used for the organic layer may be classified into, for example, light emitting materials, hole injection materials, hole transporting materials, electron transporting materials and electron injection materials depending on their function.

Materials forming a light emitting layer of an organic EL device may be classified into blue, green and red light emitting materials depending on their emission colors. Besides, yellow and orange light emitting materials may be used as such a light emitting material to realize better natural colors. In addition, a host/dopant system may be employed in the light emitting material to increase color purity and luminous efficiency through energy transfer. Dopant materials may be classified into fluorescent dopants using organic materials and phosphorescent dopants using metal complex compounds which include heavy atoms such as Ir and Pt. The developed phosphorescent materials may improve the luminous efficiency theoretically up to four times as compared to fluorescent materials, so attention is given to phosphorescent dopants as well as phosphorescent host materials.

To date, NPB, BCP and Alq₃, for example, are widely known as hole injection materials, hole transporting materials, electron transporting materials, and electron injection materials, and anthracene derivatives have been reported as light emitting materials. Particularly, metal complex compounds including Ir, such as FIrpic, Ir(ppy)₃, and (acac)Ir(btp)₂, are used as phosphorescent dopant materials of blue, green, and red colors, and 4,4-dicarbazolybiphenyl, (CBP) is used as phosphorescent host materials:

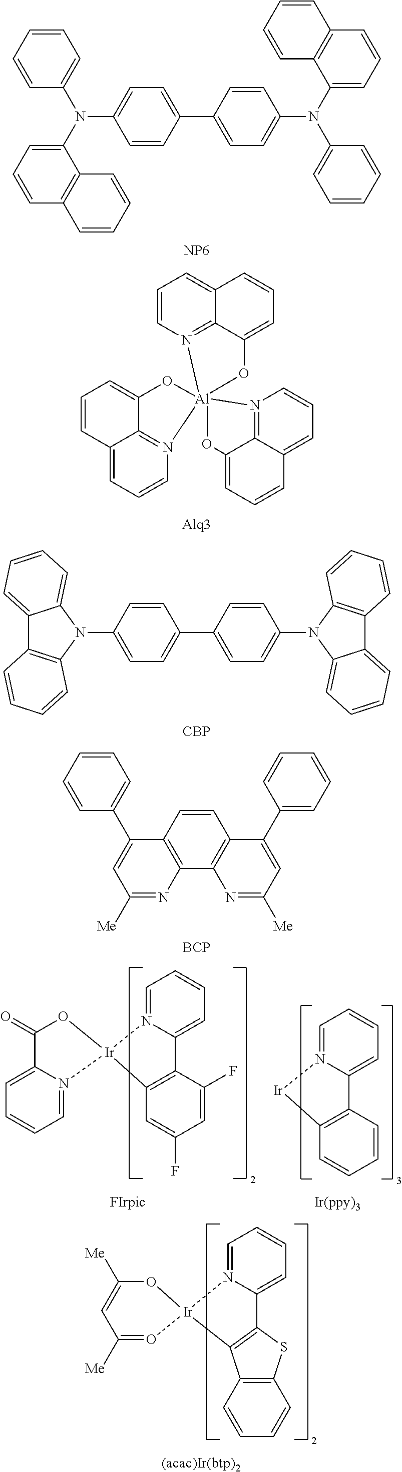

However, since conventional materials for organic layers have low glass transition temperatures, thus having poor thermal stability, and have low triplet energy, organic EL devices in which such conventional materials are used in the organic layers do not exhibit satisfactory current efficiency and lifespan characteristics. Accordingly, there is a demand for materials of organic layers that are excellent in performance.

### PRIOR ART DOCUMENT

### Patent Literature

Korean Laid-Open Patent Publication No. 2016-0078237

### DETAILED DESCRIPTION OF THE INVENTION

### Technical Objectives

The present disclosure is directed to providing a novel compound that has excellent heat resistance characteristics, carrier transporting ability and light emitting performance and thus may be used as a material for an organic layer of an organic electroluminescent device, specifically a material for a light emitting layer, a material for an electron transport auxiliary layer, a material for a light emitting auxiliary layer, or a material for an electron transporting layer.

The present disclosure is also directed to providing an organic electroluminescent device that has a low driving voltage, high luminous efficiency, and improved lifespan characteristics by including the novel compound.

### Technical Solution to the Problem

In order to achieve the above object, the present disclosure provides a compound represented by the following Chemical Formula 1:

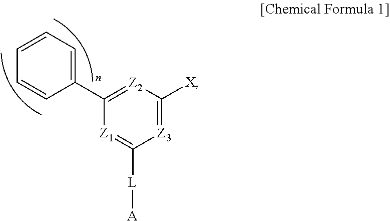

where in Chemical Formula 1,
Z₁ to Z₃ are each independently nitrogen or carbon, and include at least two nitrogens, and
X is represented by the following Chemical Formula 2 or Chemical Formula 3,

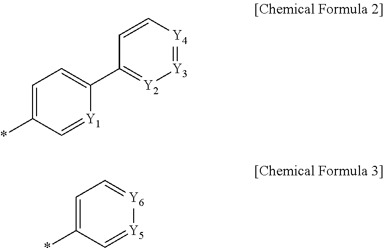

### in Chemical Formula 2 and Chemical Formula 3,

one of Y₁ to Y₄ is nitrogen and the others are carbons, and one of Y₅ and Y₆ is nitrogen and the other is carbon,
* means a site where a bond with Chemical Formula 1 is made,
n is an integer ranging from 1 to 3,
L is a single bond, or selected from the group consisting of a C₆ to C₁₈ arylene group and a heteroarylene group having 5 to 18 nuclear atoms, and
A is represented by the following Chemical Formula 4, and

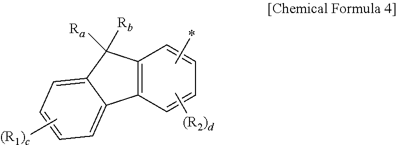

in Chemical Formula 4,
Rₐ and R_{b} are the same as or different from each other, each independently a C₁ to C₄₀ alkyl group or a C₆ to C₆₀ aryl group, or bound with each other to form a fused ring,
R₁ and R₂ are the same as or different from each other, each independently selected from the group consisting of: hydrogen, deuterium, a halogen group, a cyano group, a nitro group, an amino group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₁ to C₄₀ phosphine group, a C₁ to C₄₀ phosphine oxide group, and a C₆ to C₆₀ arylamine group, or bound with an adjacent group to form a fused ring,
c is an integer ranging from 0 to 4,
d is an integer ranging from 0 to 3,
* means a site where a bond with Chemical Formula 1 is made,
the alkyl group and the aryl group of Rₐ and R_{b}, the alkyl group, the alkenyl group, the alkynyl group, the cycloalkyl group, the heterocycloalkyl group, the aryl group, the heteroaryl group, the alkyloxy group, the aryloxy group, the alkylsilyl group, the arylsilyl group, the alkylboron group, the arylboron group, the phosphine group, the phosphine oxide group, and the arylamine group of R₁ and R₂, and the arylene group and the heteroarylene group of L are each independently substituted or unsubstituted with one or more kinds of substituents selected from the group consisting of: deuterium, a halogen group, a cyano group, a nitro group, an amino group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₁ to C₄₀ phosphine group, a C₁ to C₄₀ phosphine oxide group, and a C₆ to C₆₀ arylamine group, and when the substituents are plural in number, the plurality of substituents are the same as or different from each other.

The present disclosure also provides an organic electroluminescent device that includes an anode, a cathode and one or more organic layers disposed between the anode and the cathode. At least one of the one or more organic layers includes the compound represented by Chemical Formula 1. The organic layer including the compound represented by Chemical Formula 1 may be selected from the group consisting of: a hole injection layer, a hole transporting layer, a light emitting auxiliary layer, a light emitting layer, an electron transporting layer, and an electron injection layer. In such a case, the compound represented by Chemical Formula 1 may be used as a material of an electron transporting layer and an electron transport auxiliary layer.

### Effects of the Invention

A compound represented by Chemical Formula 1 may be used as a material for an organic layer of an organic electroluminescent device by virtue of its excellent heat resistance characteristics, carrier transporting ability and light emitting performance.

In addition, an organic electroluminescent device including the compound according to an embodiment of the present disclosure may be greatly improved in terms of light emitting performance, driving voltage, lifespan, efficiency, etc., and such an organic electroluminescent device may be effectively applied to a full color display panel and the like.

### MODES FOR CARRYING OUT THE INVENTION

Hereinafter, embodiments of the present disclosure will be described in detail.

### <Organic Compound>

A novel organic compound according to the present disclosure is a compound, represented by the above Chemical Formula 1, that has a structure, as a basic skeleton, in which a fluorene moiety is bound to an electron withdrawing group (EWG) where a pyridine moiety is bound to triazine or pyrimidine.

The compound represented by Chemical Formula 1 not only is electrochemically stable and excellent in electron transporting properties but also has high triplet energy, excellent glass transition temperature and improved thermal stability, because pyrimidine (or triazine) that has excellent electron withdrawing characteristics is bound to pyridine moiety therein. In addition, since the compound represented by Chemical Formula 1 has a higher molecular weight than that of materials of conventional organic EL devices, it has a high glass transition temperature and excellent thermal stability.

Accordingly, since the compound represented by Chemical Formula 1 has excellent electron transporting ability and luminescence properties, it may be used as a material of one of a hole injection layer, a hole transporting layer, a light emitting layer, an electron transporting layer, and an electron injection layer, which are organic layers of organic EL devices. Preferably, it may be used as a material of one of a light emitting layer of green phosphorescence, an electron transporting layer, and an electron transport auxiliary layer further laminated on the electron transporting layer.

Specifically, since the compound represented by Chemical Formula 1 has a high triplet energy, due to triplet-triplet fusion (TTF) effects, it may be used as a material for the electron transport auxiliary layer, thus exhibiting greatly increased efficiency. In addition, excitons generated in the light emitting layer may be substantially prevented from being diffused into the electron transporting layer or the hole transporting layer which are adjacent to the light emitting layer. The number of excitons contributing to light emission in the light emitting layer may increase, and thus luminous efficiency of the device may be improved. Further, durability and stability of the device may be improved, and thus the lifespan of the device may be efficiently increased. The organic EL device to which such a compound represented by the above Chemical Formula 1 is applied exhibits physical characteristics that the lifespan of the organic EL device is improved because such an organic EL device is generally capable of operating at a low voltage.

Accordingly, when the compound represented by Chemical Formula 1 is used in an organic EL device, not only excellent thermal stability and carrier transporting ability (particularly, electron transporting ability and light emitting performance) may be expected, but also the driving voltage, efficiency, lifespan and the like of the device may be improved.

In addition, the compound represented by Chemical Formula 1 is considerably advantageous for electron transporting and shows long lifespan characteristics. The excellent electron transporting ability of such a compound may provide high efficiency and fast mobility in organic EL devices, and it is easy to adjust a HOMO and LUMO energy level depending on the direction or position of substituents. Accordingly, high electron transporting ability may be provided in the organic EL device using such a compound.

Specifically, the compound represented by Chemical Formula 1 according to the present disclosure may be represented by any one of the following Chemical Formula 5 to Chemical Formula 10.

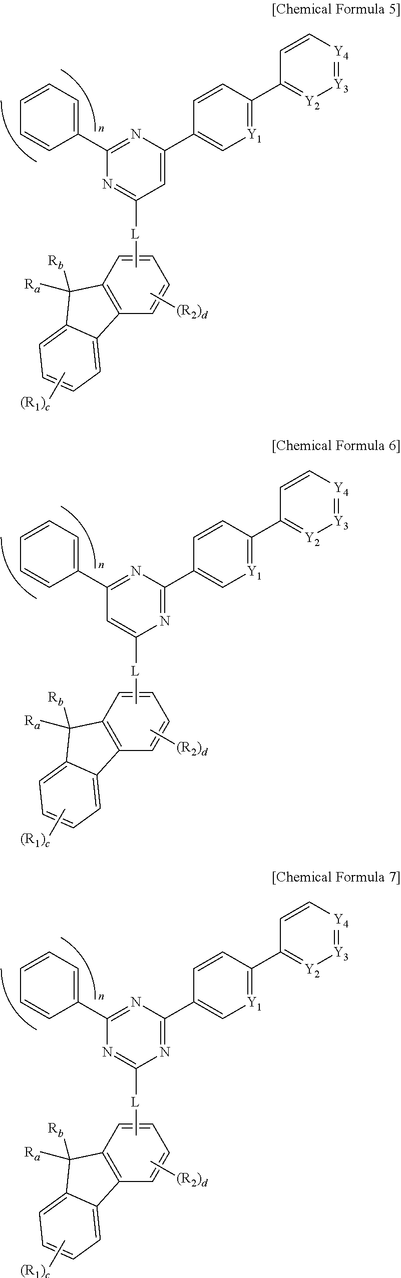

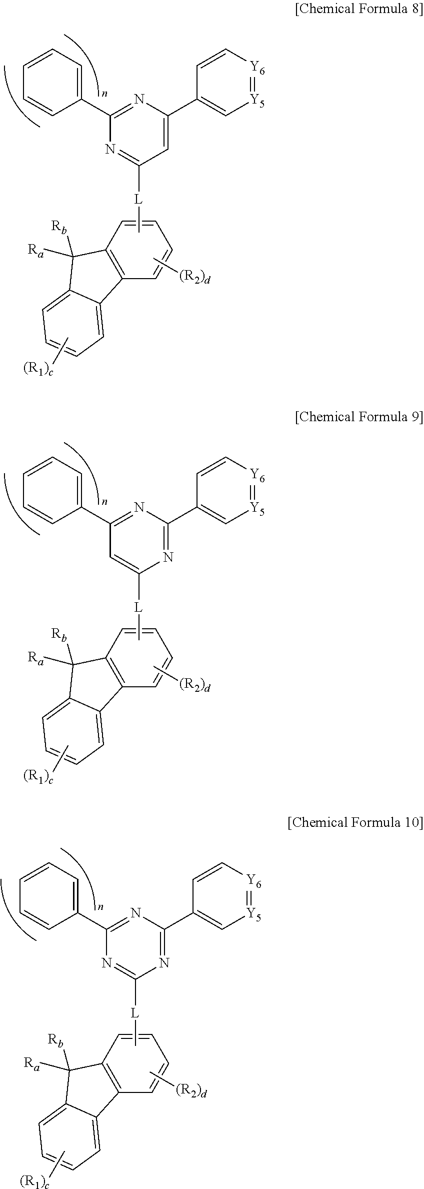

In Chemical Formula 5 to Chemical Formula 10, Rₐ, R_{b}, R₁, R₂, Y₁ to Y₆, L, c, d and n are the same as those defined in Chemical Formula 1, respectively.

Preferably, in Chemical Formula 1, X may be selected from the group consisting of the following structures represented by X-1 to X-6.

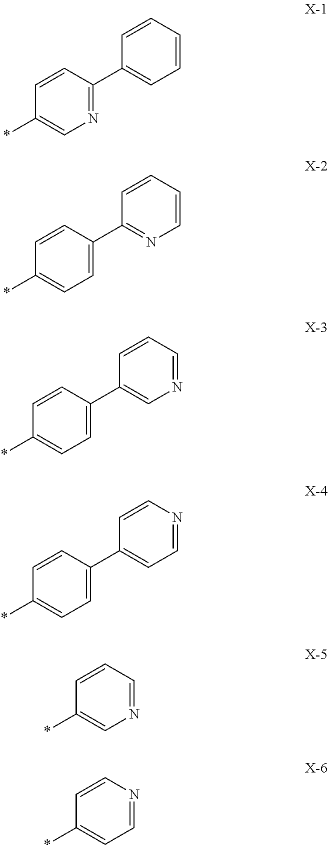 [SMILES: *c1ccc(-c2ccccc2)nc1 ; *c1ccc(-c2ccccn2)cc1 ; *c1ccc(-c2cccnc2)cc1 ; *c1ccc(-c2ccncc2)cc1 ; *c1cccnc1 ; *c1ccncc1]

Preferably, in Chemical Formula 1, a structure represented by

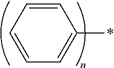

(* is a site where a bond is made) may be selected from the group consisting of the following structures represented by Ar-1 to Ar-5.

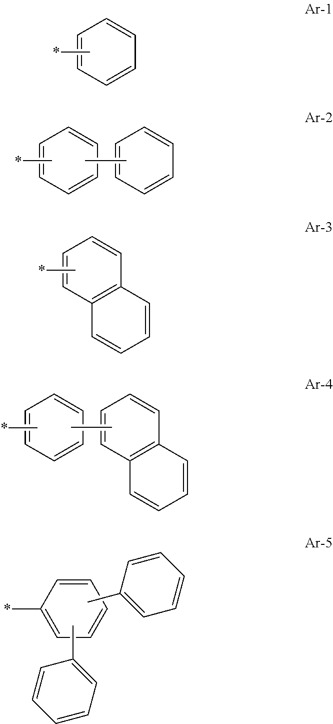

Preferably, Rₐ and R_{b} may each independently be a methyl group or a phenyl group or may be combined with each other to form a fused ring represented by

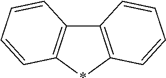 [SMILES: *1c2ccccc2c2ccccc12]

(* is a site where a bond is made).

Preferably, in Chemical Formula 1, A may be selected from the group consisting of the following structures represented by A-1 to A-6.

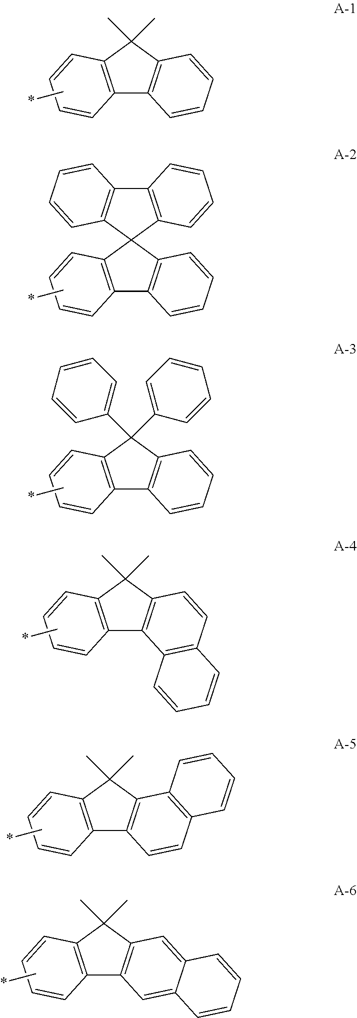

Preferably, in Chemical Formula 1, L may be a single bond or a linking group selected from the following structures represented by L-1 to L-7.

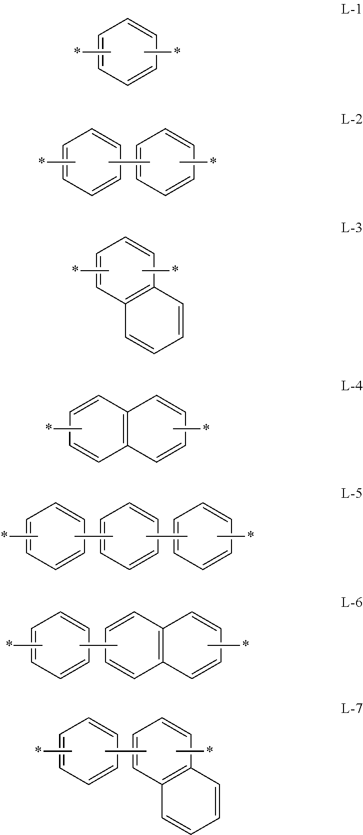

The compound, described above, represented by the above Chemical Formula 1 according to the present disclosure may be more specifically embodied as a compound represented by any one of Compounds 1 to 750 exemplified below. However, the compound represented by Chemical Formula 1 of the present disclosure is not limited by those illustrated below.

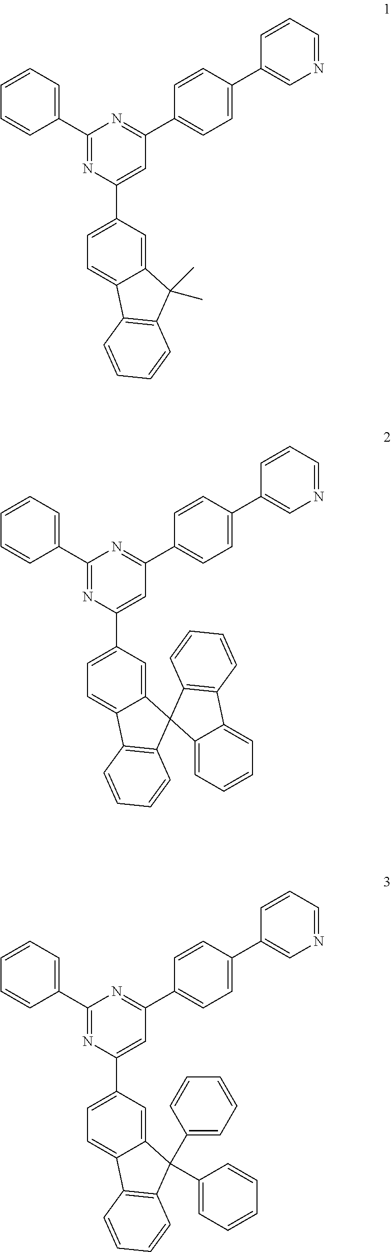 [SMILES: CC1(C)c2ccccc2-c2ccc(-c3cc(-c4ccc(-c5cccnc5)cc4)nc(-c4ccccc4)n3)cc21 ; c1ccc(-c2nc(-c3ccc(-c4cccnc4)cc3)cc(-c3ccc4c(c3)C3(c5ccccc5-c5ccccc53)c3ccccc3-4)n2)cc1 ; c1ccc(-c2nc(-c3ccc(-c4cccnc4)cc3)cc(-c3ccc4c(c3)C(c3ccccc3)(c3ccccc3)c3ccccc3-4)n2)cc1]

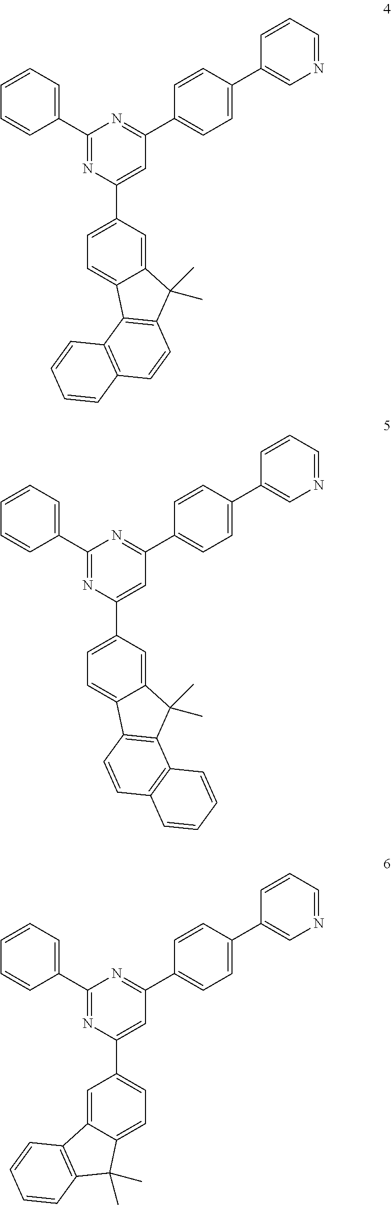 [SMILES: CC1(C)c2cc(-c3cc(-c4ccc(-c5cccnc5)cc4)nc(-c4ccccc4)n3)ccc2-c2c1ccc1ccccc21 ; CC1(C)c2cc(-c3cc(-c4ccc(-c5cccnc5)cc4)nc(-c4ccccc4)n3)ccc2-c2ccc3ccccc3c21 ; CC1(C)c2ccccc2-c2cc(-c3cc(-c4ccc(-c5cccnc5)cc4)nc(-c4ccccc4)n3)ccc21]

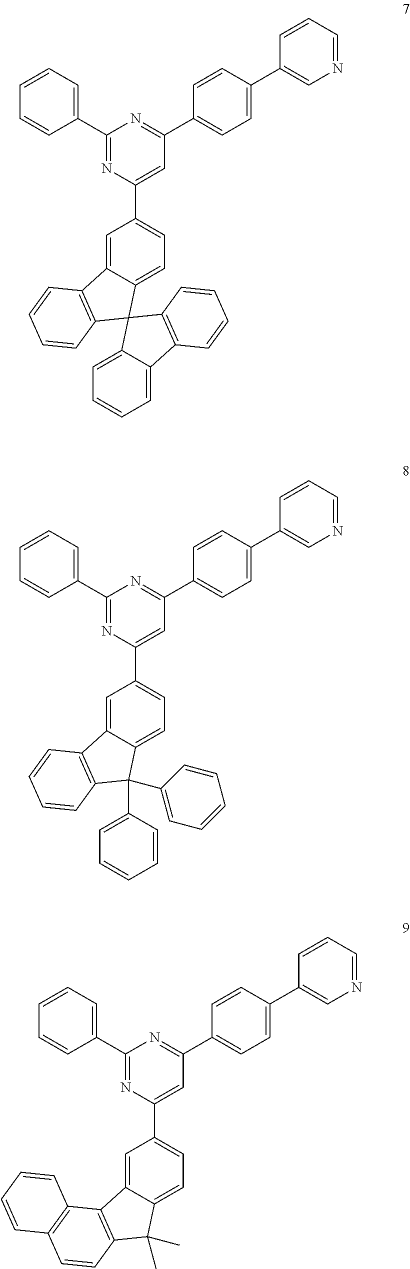 [SMILES: c1ccc(-c2nc(-c3ccc(-c4cccnc4)cc3)cc(-c3ccc4c(c3)-c3ccccc3C43c4ccccc4-c4ccccc43)n2)cc1 ; c1ccc(-c2nc(-c3ccc(-c4cccnc4)cc3)cc(-c3ccc4c(c3)-c3ccccc3C4(c3ccccc3)c3ccccc3)n2)cc1 ; CC1(C)c2ccc(-c3cc(-c4ccc(-c5cccnc5)cc4)nc(-c4ccccc4)n3)cc2-c2c1ccc1ccccc21]

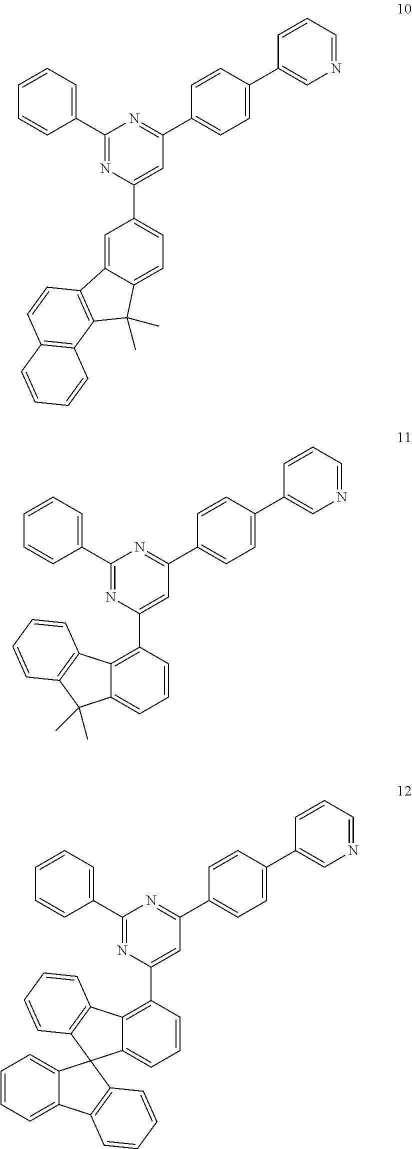 [SMILES: CC1(C)c2ccc(-c3cc(-c4ccc(-c5cccnc5)cc4)nc(-c4ccccc4)n3)cc2-c2ccc3ccccc3c21 ; CC1(C)c2ccccc2-c2c(-c3cc(-c4ccc(-c5cccnc5)cc4)nc(-c4ccccc4)n3)cccc21 ; c1ccc(-c2nc(-c3ccc(-c4cccnc4)cc3)cc(-c3cccc4c3-c3ccccc3C43c4ccccc4-c4ccccc43)n2)cc1]

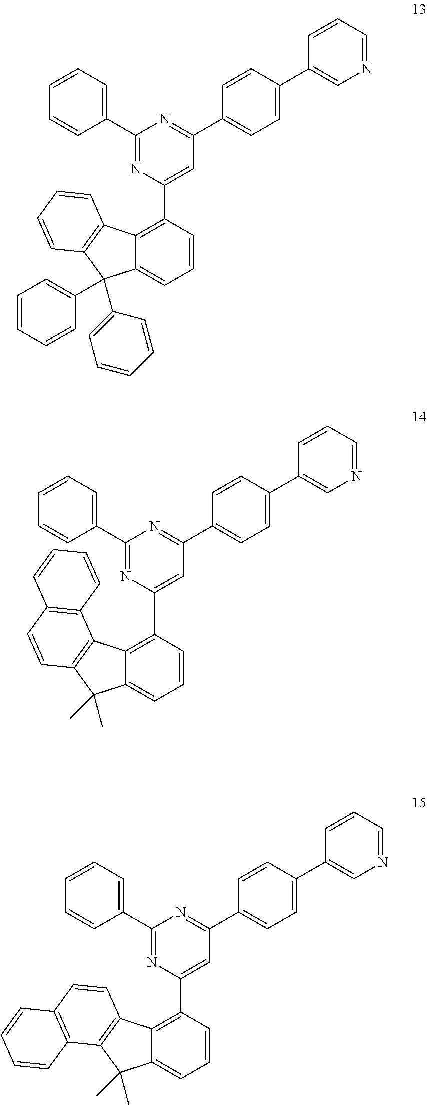 [SMILES: c1ccc(-c2nc(-c3ccc(-c4cccnc4)cc3)cc(-c3cccc4c3-c3ccccc3C4(c3ccccc3)c3ccccc3)n2)cc1 ; CC1(C)c2cccc(-c3cc(-c4ccc(-c5cccnc5)cc4)nc(-c4ccccc4)n3)c2-c2c1ccc1ccccc21 ; CC1(C)c2cccc(-c3cc(-c4ccc(-c5cccnc5)cc4)nc(-c4ccccc4)n3)c2-c2ccc3ccccc3c21]

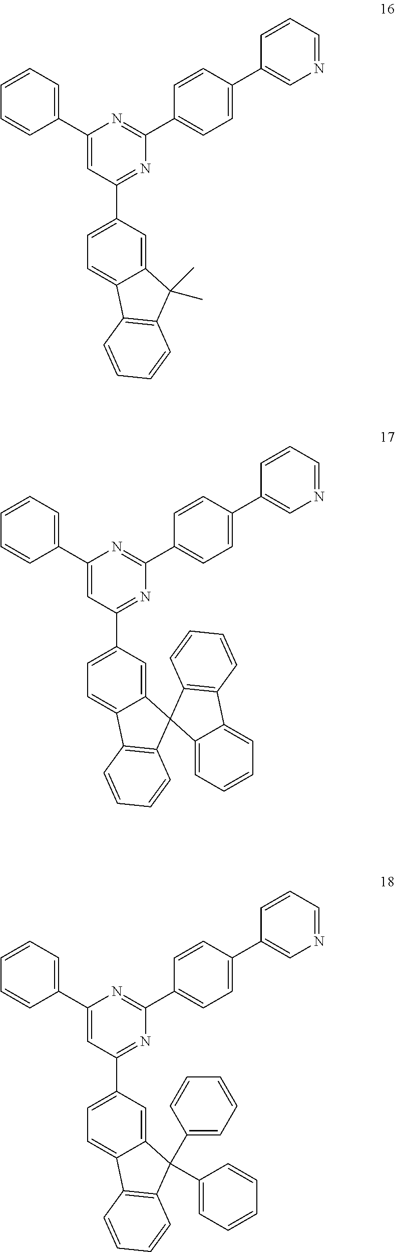 [SMILES: CC1(C)c2ccccc2-c2ccc(-c3cc(-c4ccccc4)nc(-c4ccc(-c5cccnc5)cc4)n3)cc21 ; c1ccc(-c2cc(-c3ccc4c(c3)C3(c5ccccc5-c5ccccc53)c3ccccc3-4)nc(-c3ccc(-c4cccnc4)cc3)n2)cc1 ; c1ccc(-c2cc(-c3ccc4c(c3)C(c3ccccc3)(c3ccccc3)c3ccccc3-4)nc(-c3ccc(-c4cccnc4)cc3)n2)cc1]

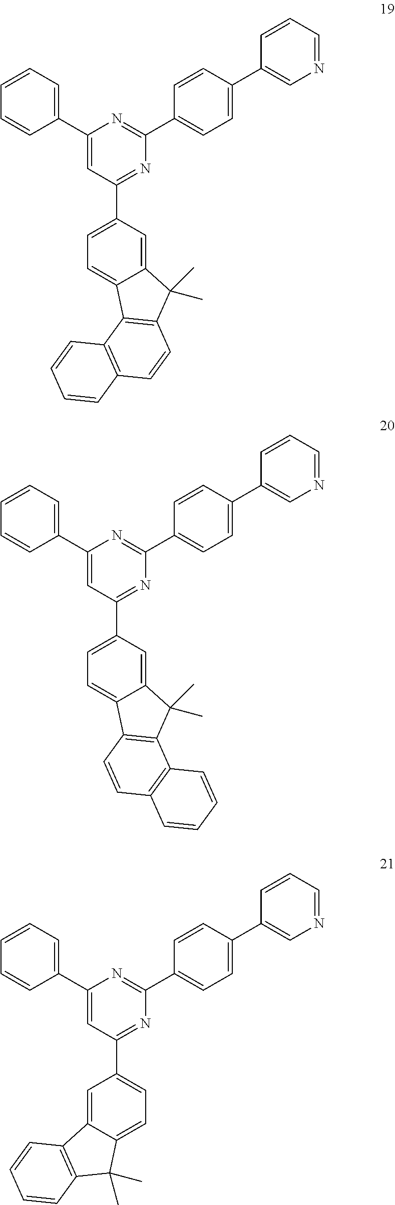 [SMILES: CC1(C)c2cc(-c3cc(-c4ccccc4)nc(-c4ccc(-c5cccnc5)cc4)n3)ccc2-c2c1ccc1ccccc21 ; CC1(C)c2cc(-c3cc(-c4ccccc4)nc(-c4ccc(-c5cccnc5)cc4)n3)ccc2-c2ccc3ccccc3c21 ; CC1(C)c2ccccc2-c2cc(-c3cc(-c4ccccc4)nc(-c4ccc(-c5cccnc5)cc4)n3)ccc21]

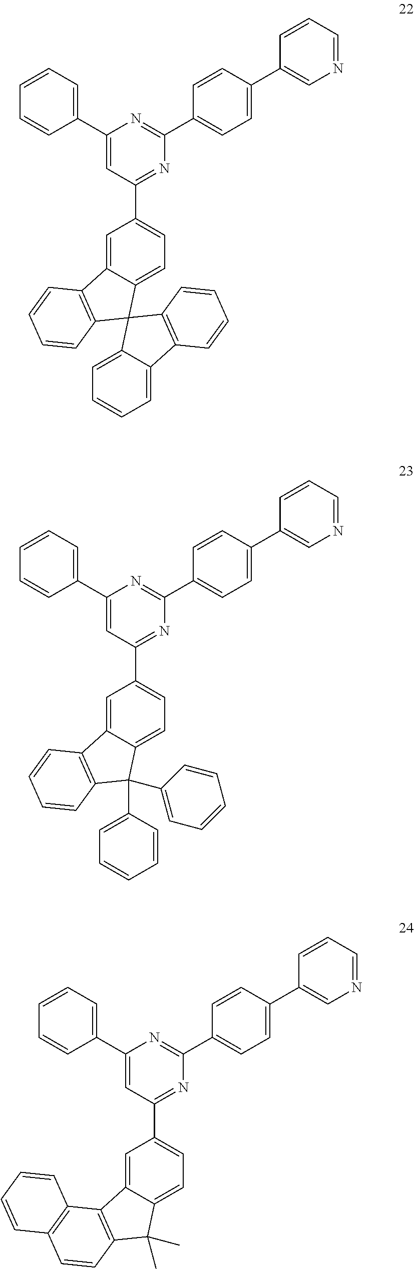 [SMILES: c1ccc(-c2cc(-c3ccc4c(c3)-c3ccccc3C43c4ccccc4-c4ccccc43)nc(-c3ccc(-c4cccnc4)cc3)n2)cc1 ; c1ccc(-c2cc(-c3ccc4c(c3)-c3ccccc3C4(c3ccccc3)c3ccccc3)nc(-c3ccc(-c4cccnc4)cc3)n2)cc1 ; CC1(C)c2ccc(-c3cc(-c4ccccc4)nc(-c4ccc(-c5cccnc5)cc4)n3)cc2-c2c1ccc1ccccc21]

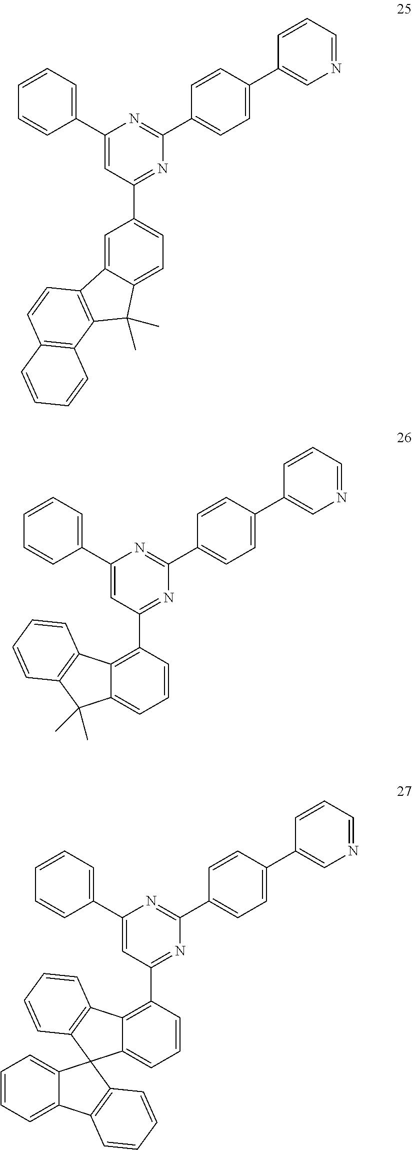 [SMILES: CC1(C)c2ccc(-c3cc(-c4ccccc4)nc(-c4ccc(-c5cccnc5)cc4)n3)cc2-c2ccc3ccccc3c21 ; CC1(C)c2ccccc2-c2c(-c3cc(-c4ccccc4)nc(-c4ccc(-c5cccnc5)cc4)n3)cccc21 ; c1ccc(-c2cc(-c3cccc4c3-c3ccccc3C43c4ccccc4-c4ccccc43)nc(-c3ccc(-c4cccnc4)cc3)n2)cc1]

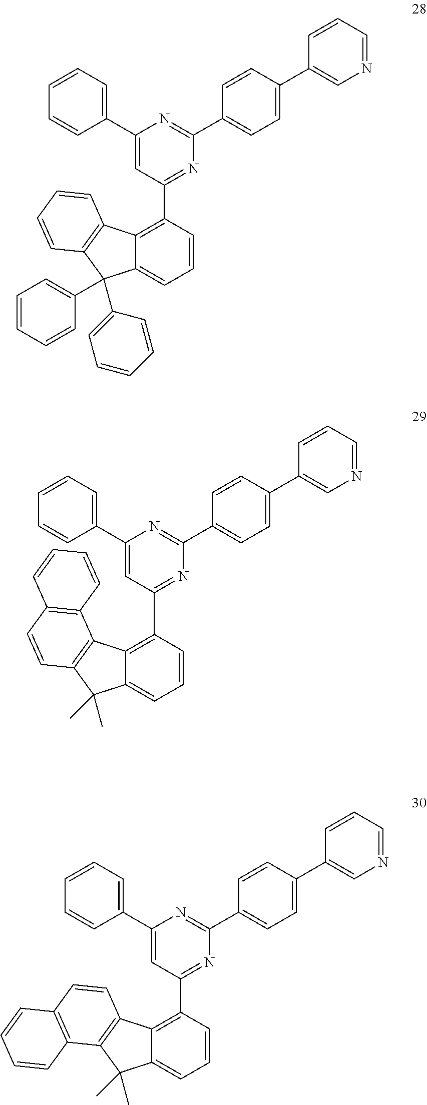 [SMILES: c1ccc(-c2cc(-c3cccc4c3-c3ccccc3C4(c3ccccc3)c3ccccc3)nc(-c3ccc(-c4cccnc4)cc3)n2)cc1 ; CC1(C)c2cccc(-c3cc(-c4ccccc4)nc(-c4ccc(-c5cccnc5)cc4)n3)c2-c2c1ccc1ccccc21 ; CC1(C)c2cccc(-c3cc(-c4ccccc4)nc(-c4ccc(-c5cccnc5)cc4)n3)c2-c2ccc3ccccc3c21]

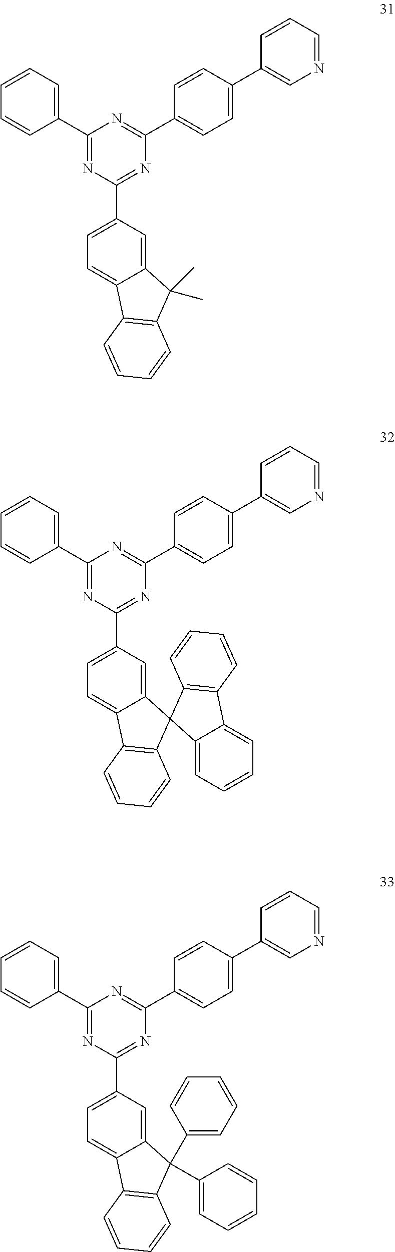 [SMILES: CC1(C)c2ccccc2-c2ccc(-c3nc(-c4ccccc4)nc(-c4ccc(-c5cccnc5)cc4)n3)cc21 ; c1ccc(-c2nc(-c3ccc(-c4cccnc4)cc3)nc(-c3ccc4c(c3)C3(c5ccccc5-c5ccccc53)c3ccccc3-4)n2)cc1 ; c1ccc(-c2nc(-c3ccc(-c4cccnc4)cc3)nc(-c3ccc4c(c3)C(c3ccccc3)(c3ccccc3)c3ccccc3-4)n2)cc1]

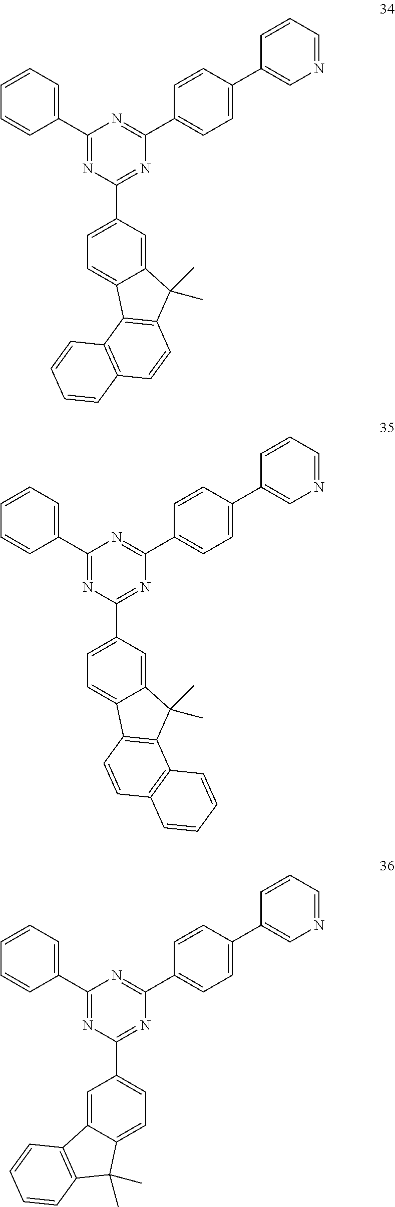 [SMILES: CC1(C)c2cc(-c3nc(-c4ccccc4)nc(-c4ccc(-c5cccnc5)cc4)n3)ccc2-c2c1ccc1ccccc21 ; CC1(C)c2cc(-c3nc(-c4ccccc4)nc(-c4ccc(-c5cccnc5)cc4)n3)ccc2-c2ccc3ccccc3c21 ; CC1(C)c2ccccc2-c2cc(-c3nc(-c4ccccc4)nc(-c4ccc(-c5cccnc5)cc4)n3)ccc21]

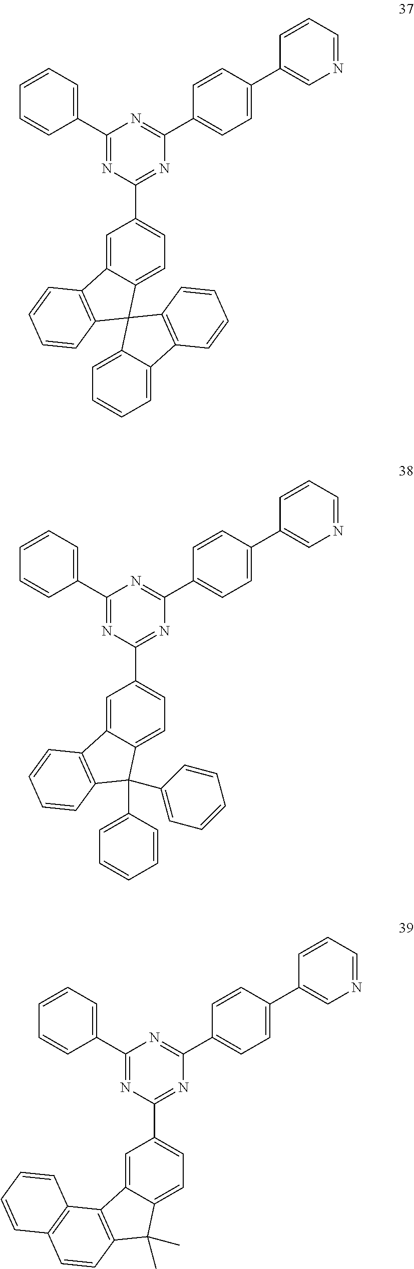 [SMILES: c1ccc(-c2nc(-c3ccc(-c4cccnc4)cc3)nc(-c3ccc4c(c3)-c3ccccc3C43c4ccccc4-c4ccccc43)n2)cc1 ; c1ccc(-c2nc(-c3ccc(-c4cccnc4)cc3)nc(-c3ccc4c(c3)-c3ccccc3C4(c3ccccc3)c3ccccc3)n2)cc1 ; CC1(C)c2ccc(-c3nc(-c4ccccc4)nc(-c4ccc(-c5cccnc5)cc4)n3)cc2-c2c1ccc1ccccc21]

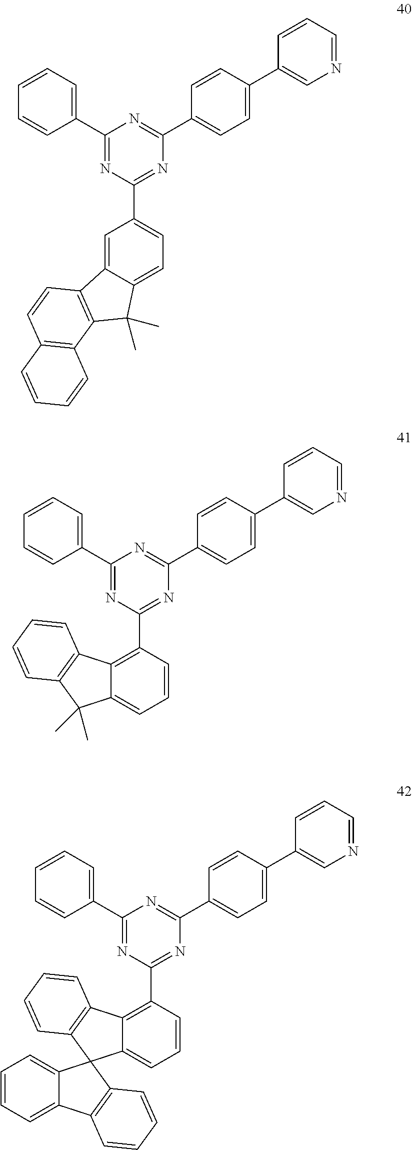 [SMILES: CC1(C)c2ccc(-c3nc(-c4ccccc4)nc(-c4ccc(-c5cccnc5)cc4)n3)cc2-c2ccc3ccccc3c21 ; CC1(C)c2ccccc2-c2c(-c3nc(-c4ccccc4)nc(-c4ccc(-c5cccnc5)cc4)n3)cccc21 ; c1ccc(-c2nc(-c3ccc(-c4cccnc4)cc3)nc(-c3cccc4c3-c3ccccc3C43c4ccccc4-c4ccccc43)n2)cc1]

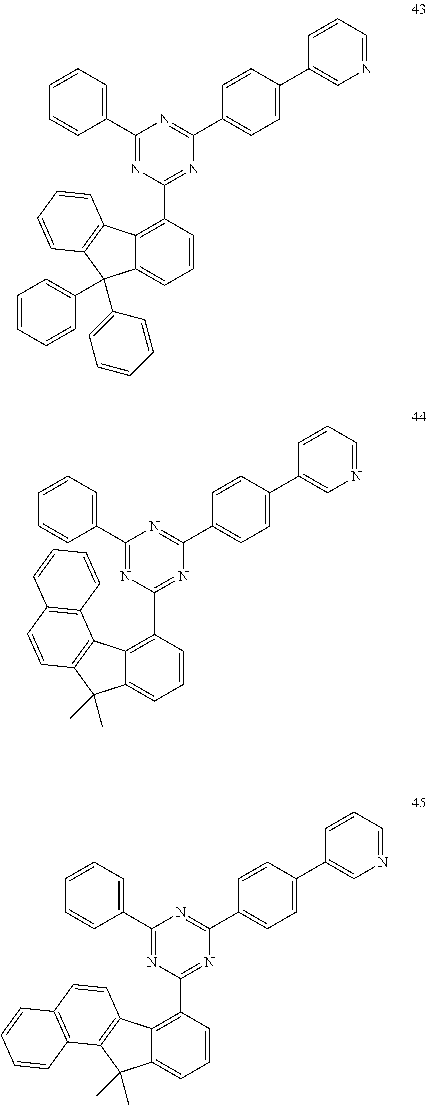 [SMILES: c1ccc(-c2nc(-c3ccc(-c4cccnc4)cc3)nc(-c3cccc4c3-c3ccccc3C4(c3ccccc3)c3ccccc3)n2)cc1 ; CC1(C)c2cccc(-c3nc(-c4ccccc4)nc(-c4ccc(-c5cccnc5)cc4)n3)c2-c2c1ccc1ccccc21 ; CC1(C)c2cccc(-c3nc(-c4ccccc4)nc(-c4ccc(-c5cccnc5)cc4)n3)c2-c2ccc3ccccc3c21]

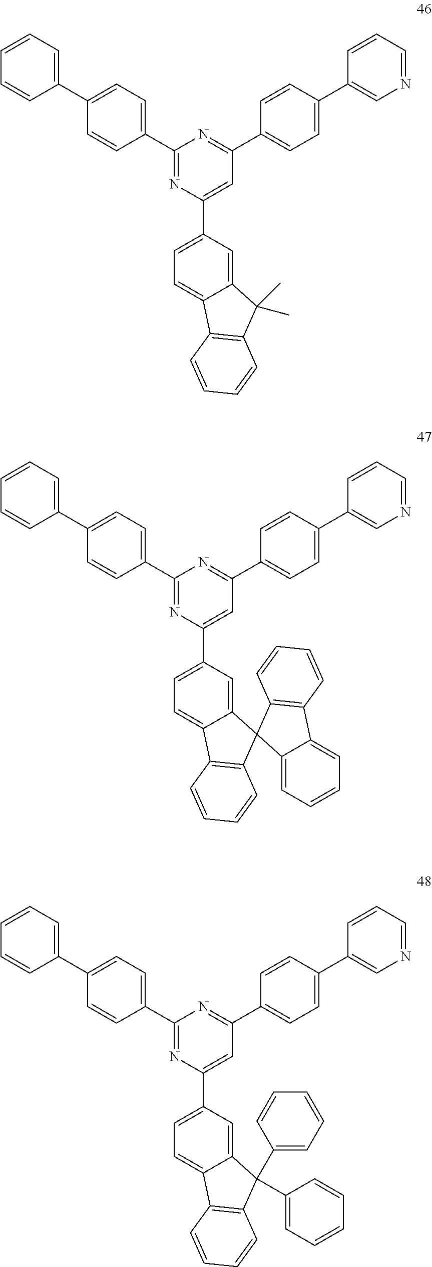 [SMILES: CC1(C)c2ccccc2-c2ccc(-c3cc(-c4ccc(-c5cccnc5)cc4)nc(-c4ccc(-c5ccccc5)cc4)n3)cc21 ; c1ccc(-c2ccc(-c3nc(-c4ccc(-c5cccnc5)cc4)cc(-c4ccc5c(c4)C4(c6ccccc6-c6ccccc64)c4ccccc4-5)n3)cc2)cc1 ; c1ccc(-c2ccc(-c3nc(-c4ccc(-c5cccnc5)cc4)cc(-c4ccc5c(c4)C(c4ccccc4)(c4ccccc4)c4ccccc4-5)n3)cc2)cc1]

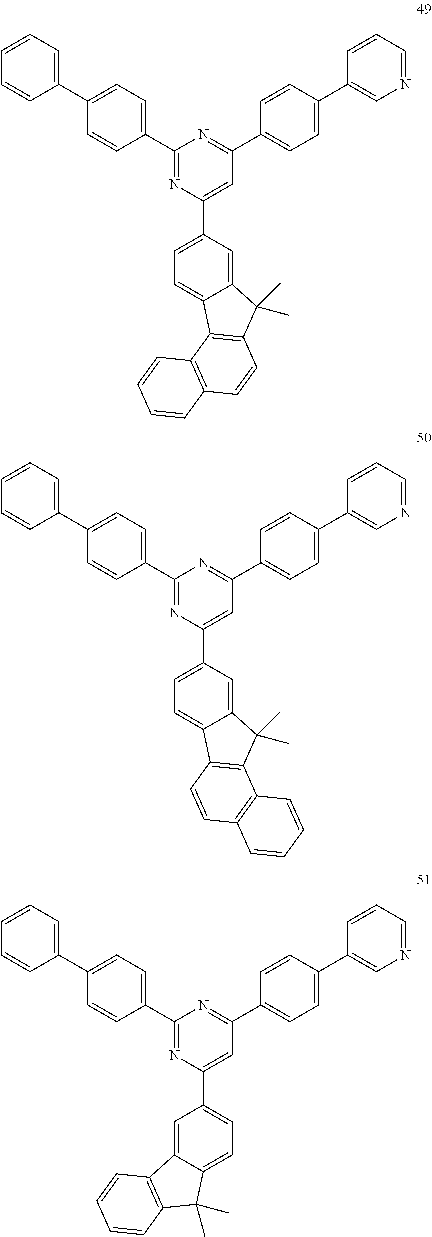 [SMILES: CC1(C)c2cc(-c3cc(-c4ccc(-c5cccnc5)cc4)nc(-c4ccc(-c5ccccc5)cc4)n3)ccc2-c2c1ccc1ccccc21 ; CC1(C)c2cc(-c3cc(-c4ccc(-c5cccnc5)cc4)nc(-c4ccc(-c5ccccc5)cc4)n3)ccc2-c2ccc3ccccc3c21 ; CC1(C)c2ccccc2-c2cc(-c3cc(-c4ccc(-c5cccnc5)cc4)nc(-c4ccc(-c5ccccc5)cc4)n3)ccc21]

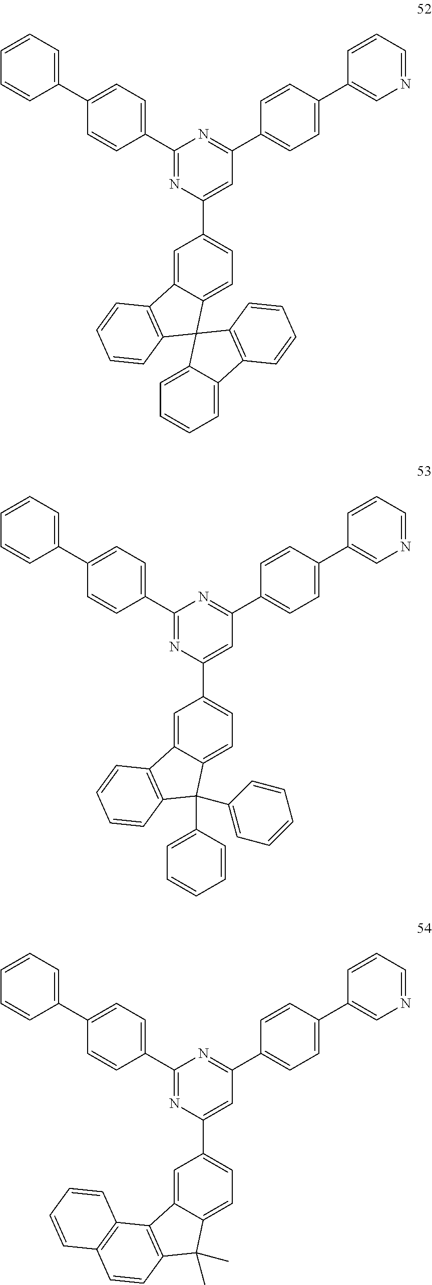 [SMILES: c1ccc(-c2ccc(-c3nc(-c4ccc(-c5cccnc5)cc4)cc(-c4ccc5c(c4)-c4ccccc4C54c5ccccc5-c5ccccc54)n3)cc2)cc1 ; c1ccc(-c2ccc(-c3nc(-c4ccc(-c5cccnc5)cc4)cc(-c4ccc5c(c4)-c4ccccc4C5(c4ccccc4)c4ccccc4)n3)cc2)cc1 ; CC1(C)c2ccc(-c3cc(-c4ccc(-c5cccnc5)cc4)nc(-c4ccc(-c5ccccc5)cc4)n3)cc2-c2c1ccc1ccccc21]

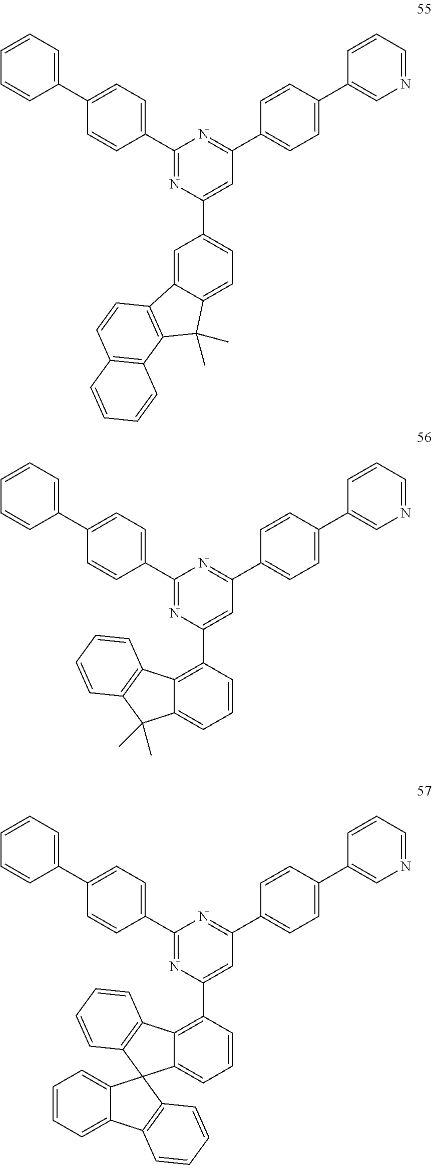 [SMILES: CC1(C)c2ccc(-c3cc(-c4ccc(-c5cccnc5)cc4)nc(-c4ccc(-c5ccccc5)cc4)n3)cc2-c2ccc3ccccc3c21 ; CC1(C)c2ccccc2-c2c(-c3cc(-c4ccc(-c5cccnc5)cc4)nc(-c4ccc(-c5ccccc5)cc4)n3)cccc21 ; c1ccc(-c2ccc(-c3nc(-c4ccc(-c5cccnc5)cc4)cc(-c4cccc5c4-c4ccccc4C54c5ccccc5-c5ccccc54)n3)cc2)cc1]

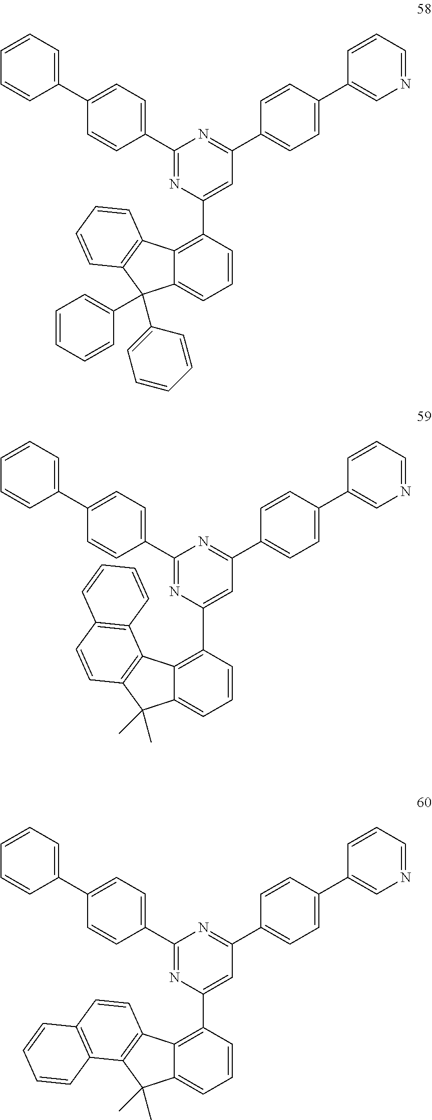 [SMILES: c1ccc(-c2ccc(-c3nc(-c4ccc(-c5cccnc5)cc4)cc(-c4cccc5c4-c4ccccc4C5(c4ccccc4)c4ccccc4)n3)cc2)cc1 ; CC1(C)c2cccc(-c3cc(-c4ccc(-c5cccnc5)cc4)nc(-c4ccc(-c5ccccc5)cc4)n3)c2-c2c1ccc1ccccc21 ; CC1(C)c2cccc(-c3cc(-c4ccc(-c5cccnc5)cc4)nc(-c4ccc(-c5ccccc5)cc4)n3)c2-c2ccc3ccccc3c21]

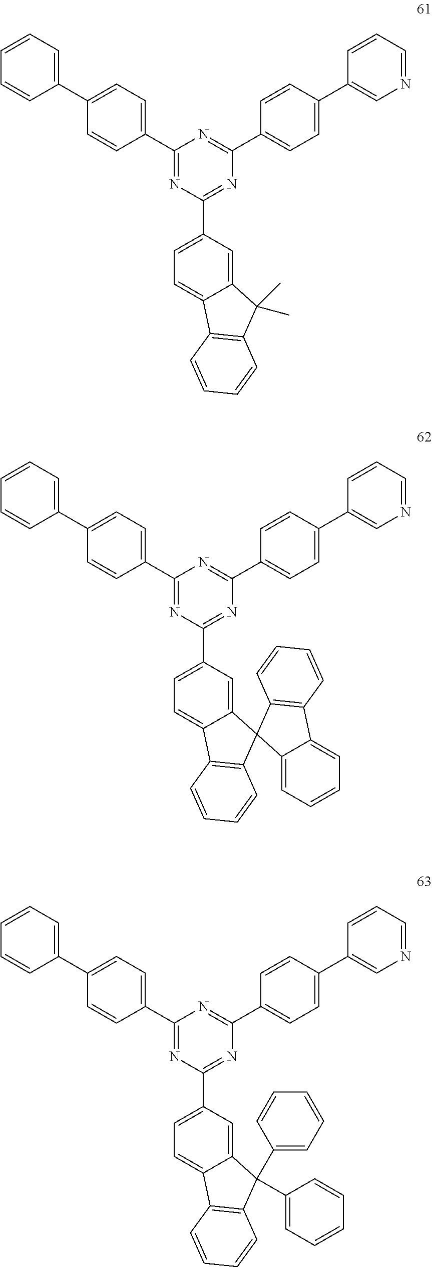 [SMILES: CC1(C)c2ccccc2-c2ccc(-c3nc(-c4ccc(-c5ccccc5)cc4)nc(-c4ccc(-c5cccnc5)cc4)n3)cc21 ; c1ccc(-c2ccc(-c3nc(-c4ccc(-c5cccnc5)cc4)nc(-c4ccc5c(c4)C4(c6ccccc6-c6ccccc64)c4ccccc4-5)n3)cc2)cc1 ; c1ccc(-c2ccc(-c3nc(-c4ccc(-c5cccnc5)cc4)nc(-c4ccc5c(c4)C(c4ccccc4)(c4ccccc4)c4ccccc4-5)n3)cc2)cc1]

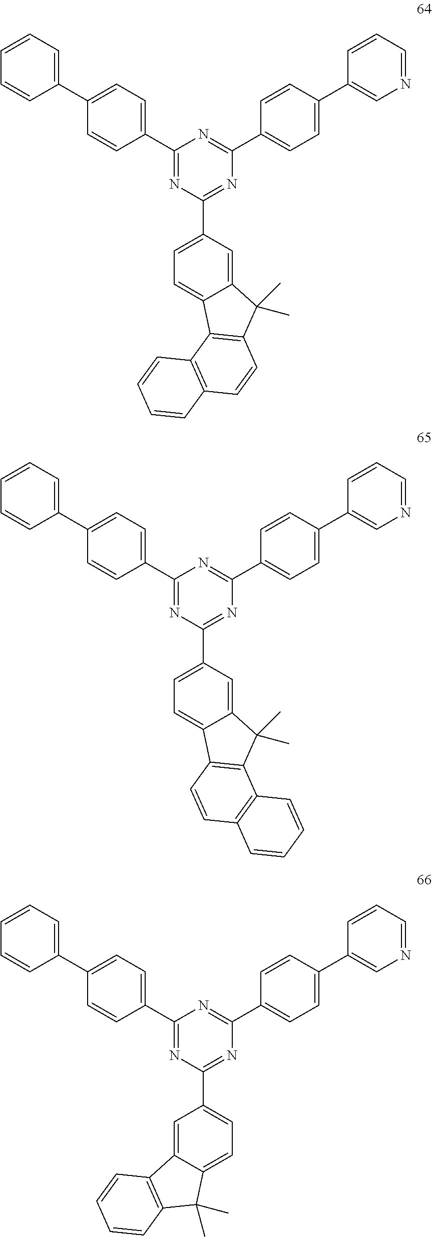 [SMILES: CC1(C)c2cc(-c3nc(-c4ccc(-c5ccccc5)cc4)nc(-c4ccc(-c5cccnc5)cc4)n3)ccc2-c2c1ccc1ccccc21 ; CC1(C)c2cc(-c3nc(-c4ccc(-c5ccccc5)cc4)nc(-c4ccc(-c5cccnc5)cc4)n3)ccc2-c2ccc3ccccc3c21 ; CC1(C)c2ccccc2-c2cc(-c3nc(-c4ccc(-c5ccccc5)cc4)nc(-c4ccc(-c5cccnc5)cc4)n3)ccc21]

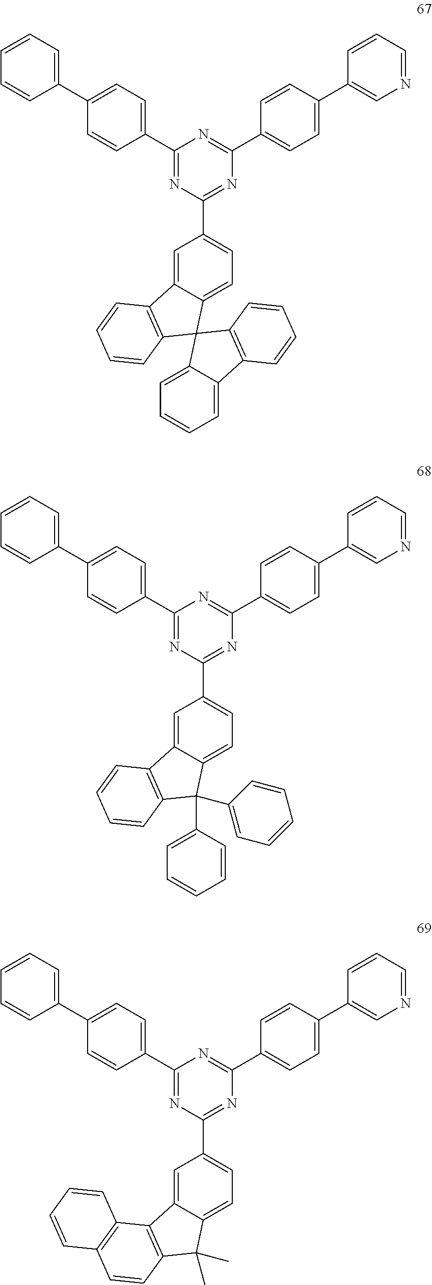 [SMILES: c1ccc(-c2ccc(-c3nc(-c4ccc(-c5cccnc5)cc4)nc(-c4ccc5c(c4)-c4ccccc4C54c5ccccc5-c5ccccc54)n3)cc2)cc1 ; c1ccc(-c2ccc(-c3nc(-c4ccc(-c5cccnc5)cc4)nc(-c4ccc5c(c4)-c4ccccc4C5(c4ccccc4)c4ccccc4)n3)cc2)cc1 ; CC1(C)c2ccc(-c3nc(-c4ccc(-c5ccccc5)cc4)nc(-c4ccc(-c5cccnc5)cc4)n3)cc2-c2c1ccc1ccccc21]

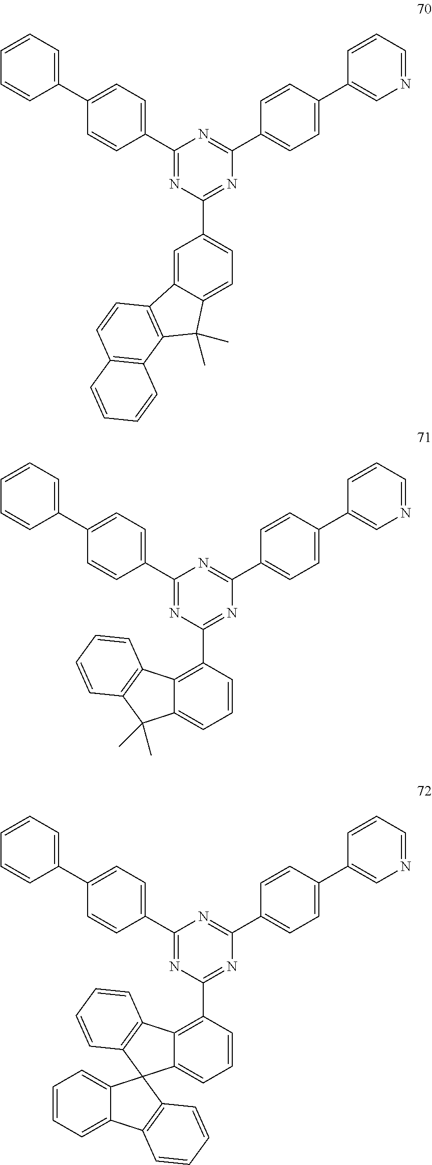 [SMILES: CC1(C)c2ccc(-c3nc(-c4ccc(-c5ccccc5)cc4)nc(-c4ccc(-c5cccnc5)cc4)n3)cc2-c2ccc3ccccc3c21 ; CC1(C)c2ccccc2-c2c(-c3nc(-c4ccc(-c5ccccc5)cc4)nc(-c4ccc(-c5cccnc5)cc4)n3)cccc21 ; c1ccc(-c2ccc(-c3nc(-c4ccc(-c5cccnc5)cc4)nc(-c4cccc5c4-c4ccccc4C54c5ccccc5-c5ccccc54)n3)cc2)cc1]

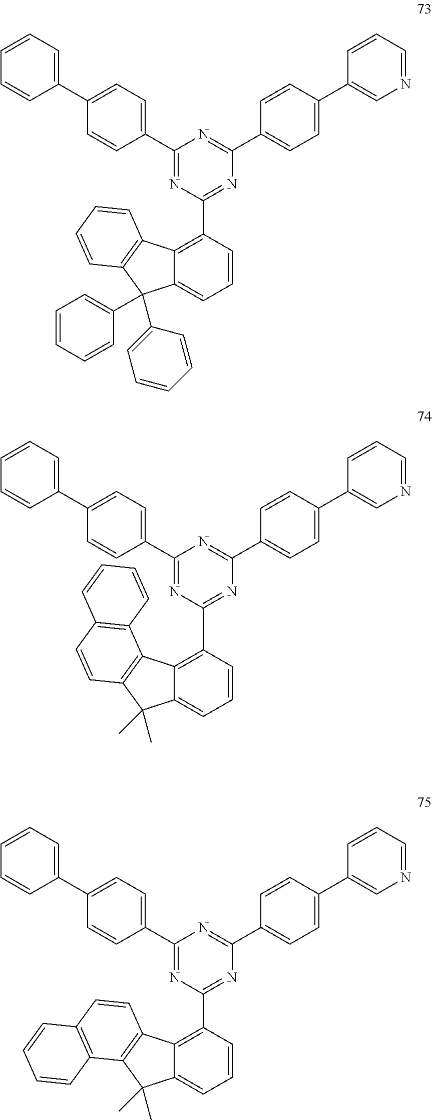 [SMILES: c1ccc(-c2ccc(-c3nc(-c4ccc(-c5cccnc5)cc4)nc(-c4cccc5c4-c4ccccc4C5(c4ccccc4)c4ccccc4)n3)cc2)cc1 ; CC1(C)c2cccc(-c3nc(-c4ccc(-c5ccccc5)cc4)nc(-c4ccc(-c5cccnc5)cc4)n3)c2-c2c1ccc1ccccc21 ; CC1(C)c2cccc(-c3nc(-c4ccc(-c5ccccc5)cc4)nc(-c4ccc(-c5cccnc5)cc4)n3)c2-c2ccc3ccccc3c21]

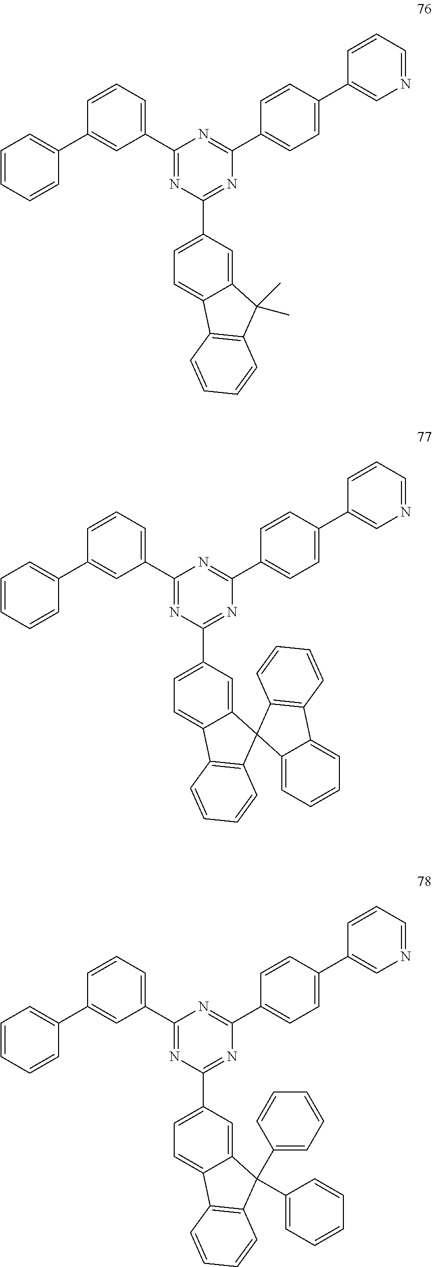 [SMILES: CC1(C)c2ccccc2-c2ccc(-c3nc(-c4ccc(-c5cccnc5)cc4)nc(-c4cccc(-c5ccccc5)c4)n3)cc21 ; c1ccc(-c2cccc(-c3nc(-c4ccc(-c5cccnc5)cc4)nc(-c4ccc5c(c4)C4(c6ccccc6-c6ccccc64)c4ccccc4-5)n3)c2)cc1 ; c1ccc(-c2cccc(-c3nc(-c4ccc(-c5cccnc5)cc4)nc(-c4ccc5c(c4)C(c4ccccc4)(c4ccccc4)c4ccccc4-5)n3)c2)cc1]

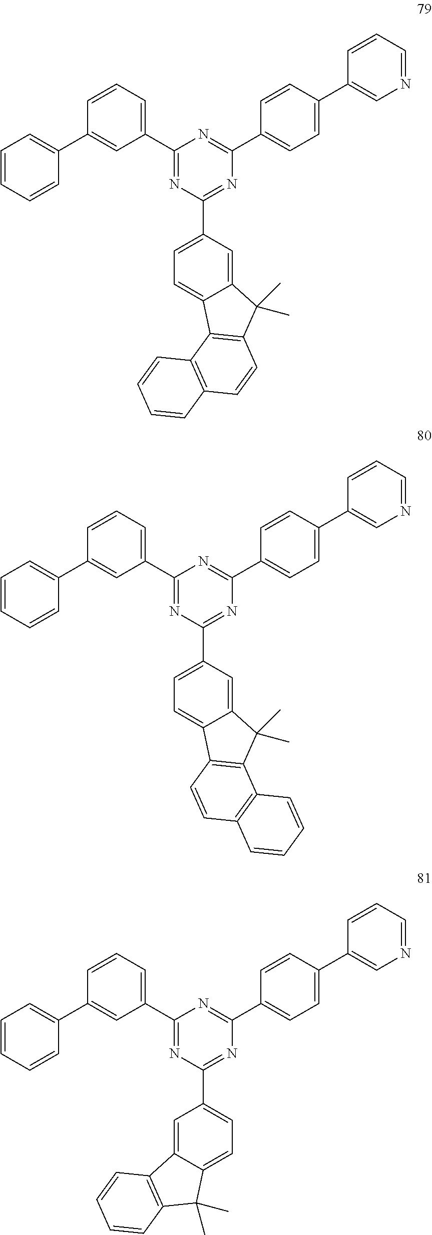 [SMILES: CC1(C)c2cc(-c3nc(-c4ccc(-c5cccnc5)cc4)nc(-c4cccc(-c5ccccc5)c4)n3)ccc2-c2c1ccc1ccccc21 ; CC1(C)c2cc(-c3nc(-c4ccc(-c5cccnc5)cc4)nc(-c4cccc(-c5ccccc5)c4)n3)ccc2-c2ccc3ccccc3c21 ; CC1(C)c2ccccc2-c2cc(-c3nc(-c4ccc(-c5cccnc5)cc4)nc(-c4cccc(-c5ccccc5)c4)n3)ccc21]

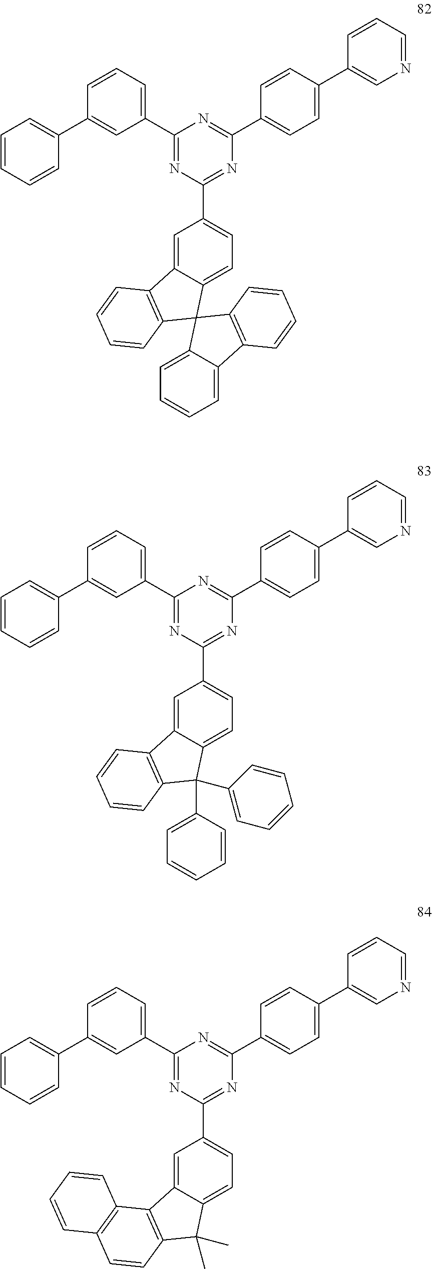 [SMILES: c1ccc(-c2cccc(-c3nc(-c4ccc(-c5cccnc5)cc4)nc(-c4ccc5c(c4)-c4ccccc4C54c5ccccc5-c5ccccc54)n3)c2)cc1 ; c1ccc(-c2cccc(-c3nc(-c4ccc(-c5cccnc5)cc4)nc(-c4ccc5c(c4)-c4ccccc4C5(c4ccccc4)c4ccccc4)n3)c2)cc1 ; CC1(C)c2ccc(-c3nc(-c4ccc(-c5cccnc5)cc4)nc(-c4cccc(-c5ccccc5)c4)n3)cc2-c2c1ccc1ccccc21]

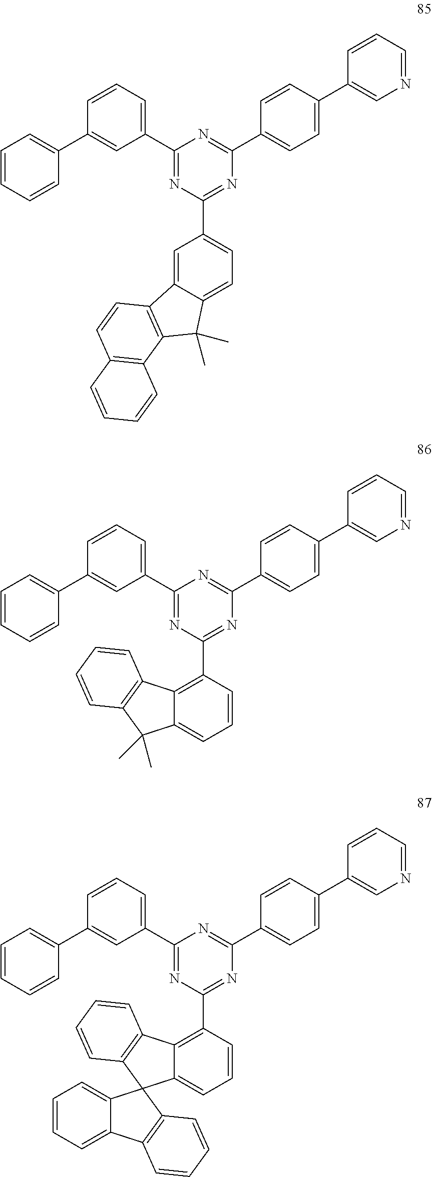 [SMILES: CC1(C)c2ccc(-c3nc(-c4ccc(-c5cccnc5)cc4)nc(-c4cccc(-c5ccccc5)c4)n3)cc2-c2ccc3ccccc3c21 ; CC1(C)c2ccccc2-c2c(-c3nc(-c4ccc(-c5cccnc5)cc4)nc(-c4cccc(-c5ccccc5)c4)n3)cccc21 ; c1ccc(-c2cccc(-c3nc(-c4ccc(-c5cccnc5)cc4)nc(-c4cccc5c4-c4ccccc4C54c5ccccc5-c5ccccc54)n3)c2)cc1]

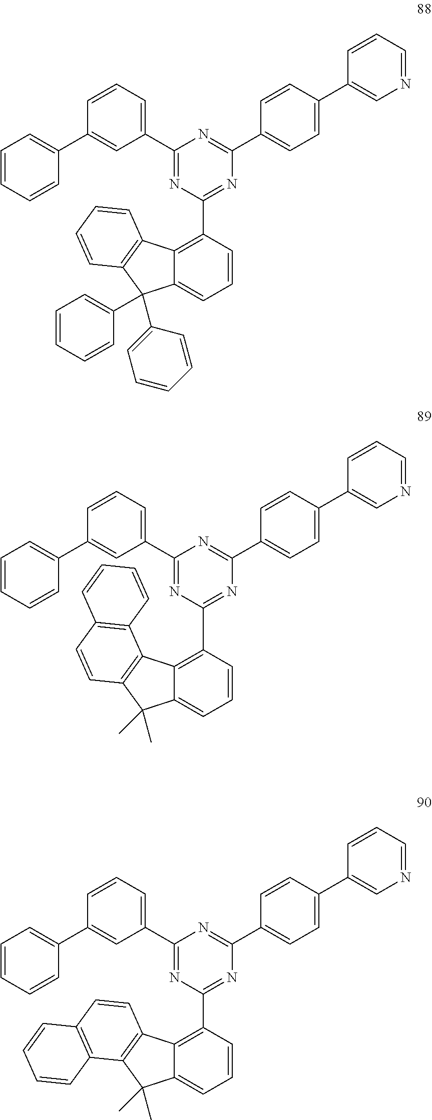 [SMILES: c1ccc(-c2cccc(-c3nc(-c4ccc(-c5cccnc5)cc4)nc(-c4cccc5c4-c4ccccc4C5(c4ccccc4)c4ccccc4)n3)c2)cc1 ; CC1(C)c2cccc(-c3nc(-c4ccc(-c5cccnc5)cc4)nc(-c4cccc(-c5ccccc5)c4)n3)c2-c2c1ccc1ccccc21 ; CC1(C)c2cccc(-c3nc(-c4ccc(-c5cccnc5)cc4)nc(-c4cccc(-c5ccccc5)c4)n3)c2-c2ccc3ccccc3c21]

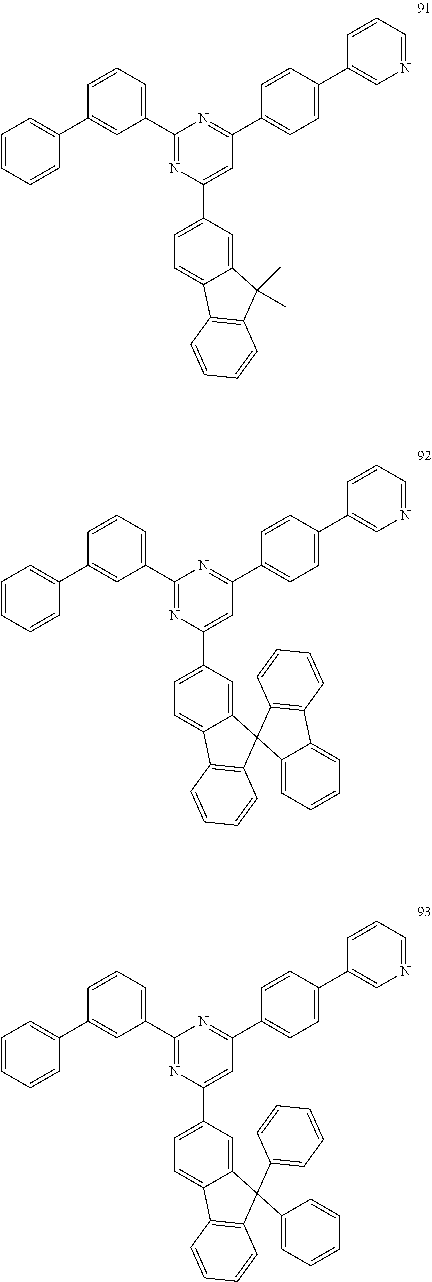 [SMILES: CC1(C)c2ccccc2-c2ccc(-c3cc(-c4ccc(-c5cccnc5)cc4)nc(-c4cccc(-c5ccccc5)c4)n3)cc21 ; c1ccc(-c2cccc(-c3nc(-c4ccc(-c5cccnc5)cc4)cc(-c4ccc5c(c4)C4(c6ccccc6-c6ccccc64)c4ccccc4-5)n3)c2)cc1 ; c1ccc(-c2cccc(-c3nc(-c4ccc(-c5cccnc5)cc4)cc(-c4ccc5c(c4)C(c4ccccc4)(c4ccccc4)c4ccccc4-5)n3)c2)cc1]

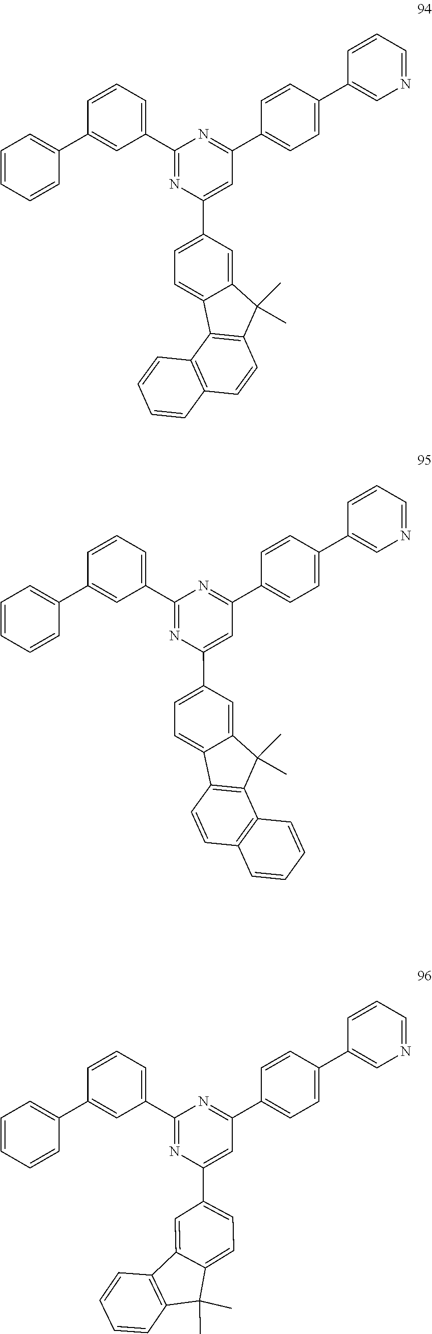 [SMILES: CC1(C)c2cc(-c3cc(-c4ccc(-c5cccnc5)cc4)nc(-c4cccc(-c5ccccc5)c4)n3)ccc2-c2c1ccc1ccccc21 ; CC1(C)c2cc(-c3cc(-c4ccc(-c5cccnc5)cc4)nc(-c4cccc(-c5ccccc5)c4)n3)ccc2-c2ccc3ccccc3c21 ; CC1(C)c2ccccc2-c2cc(-c3cc(-c4ccc(-c5cccnc5)cc4)nc(-c4cccc(-c5ccccc5)c4)n3)ccc21]

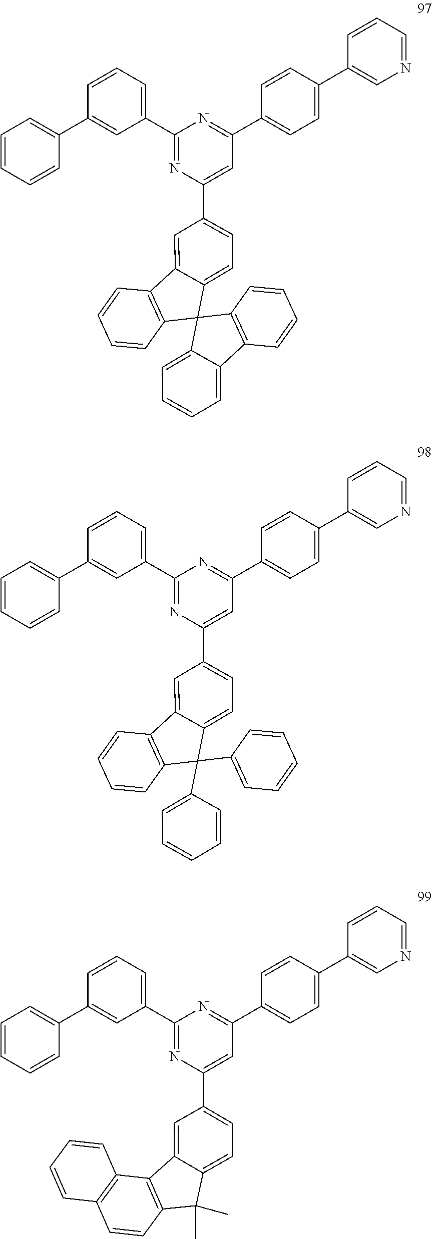 [SMILES: c1ccc(-c2cccc(-c3nc(-c4ccc(-c5cccnc5)cc4)cc(-c4ccc5c(c4)-c4ccccc4C54c5ccccc5-c5ccccc54)n3)c2)cc1 ; c1ccc(-c2cccc(-c3nc(-c4ccc(-c5cccnc5)cc4)cc(-c4ccc5c(c4)-c4ccccc4C5(c4ccccc4)c4ccccc4)n3)c2)cc1 ; CC1(C)c2ccc(-c3cc(-c4ccc(-c5cccnc5)cc4)nc(-c4cccc(-c5ccccc5)c4)n3)cc2-c2c1ccc1ccccc21]

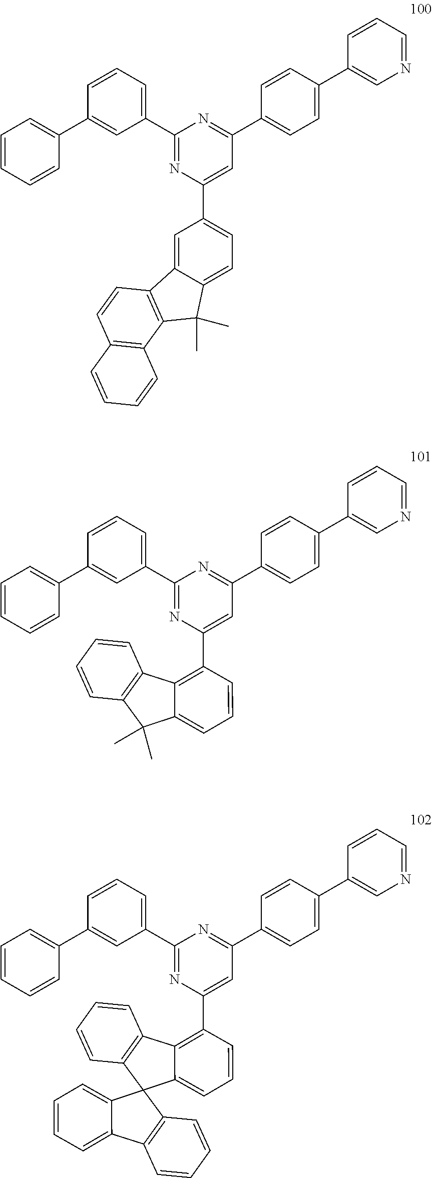 [SMILES: CC1(C)c2ccc(-c3cc(-c4ccc(-c5cccnc5)cc4)nc(-c4cccc(-c5ccccc5)c4)n3)cc2-c2ccc3ccccc3c21 ; CC1(C)c2ccccc2-c2c(-c3cc(-c4ccc(-c5cccnc5)cc4)nc(-c4cccc(-c5ccccc5)c4)n3)cccc21 ; c1ccc(-c2cccc(-c3nc(-c4ccc(-c5cccnc5)cc4)cc(-c4cccc5c4-c4ccccc4C54c5ccccc5-c5ccccc54)n3)c2)cc1]

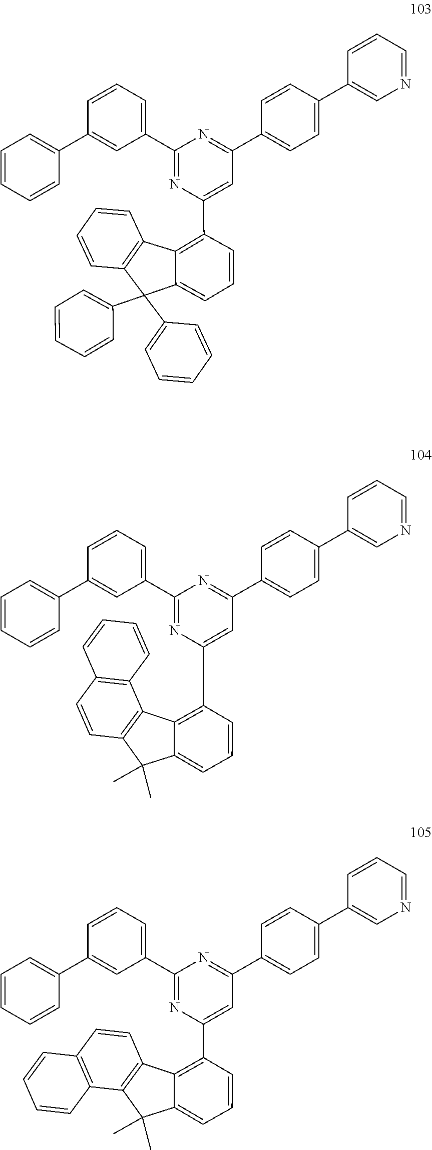 [SMILES: c1ccc(-c2cccc(-c3nc(-c4ccc(-c5cccnc5)cc4)cc(-c4cccc5c4-c4ccccc4C5(c4ccccc4)c4ccccc4)n3)c2)cc1 ; CC1(C)c2cccc(-c3cc(-c4ccc(-c5cccnc5)cc4)nc(-c4cccc(-c5ccccc5)c4)n3)c2-c2c1ccc1ccccc21 ; CC1(C)c2cccc(-c3cc(-c4ccc(-c5cccnc5)cc4)nc(-c4cccc(-c5ccccc5)c4)n3)c2-c2ccc3ccccc3c21]

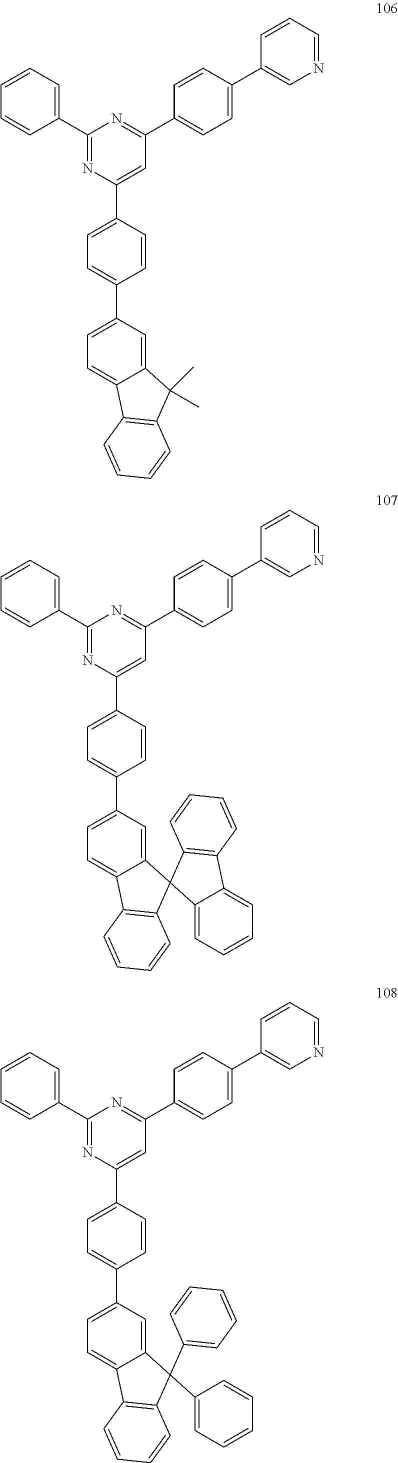 [SMILES: CC1(C)c2ccccc2-c2ccc(-c3ccc(-c4cc(-c5ccc(-c6cccnc6)cc5)nc(-c5ccccc5)n4)cc3)cc21 ; c1ccc(-c2nc(-c3ccc(-c4cccnc4)cc3)cc(-c3ccc(-c4ccc5c(c4)C4(c6ccccc6-c6ccccc64)c4ccccc4-5)cc3)n2)cc1 ; c1ccc(-c2nc(-c3ccc(-c4cccnc4)cc3)cc(-c3ccc(-c4ccc5c(c4)C(c4ccccc4)(c4ccccc4)c4ccccc4-5)cc3)n2)cc1]

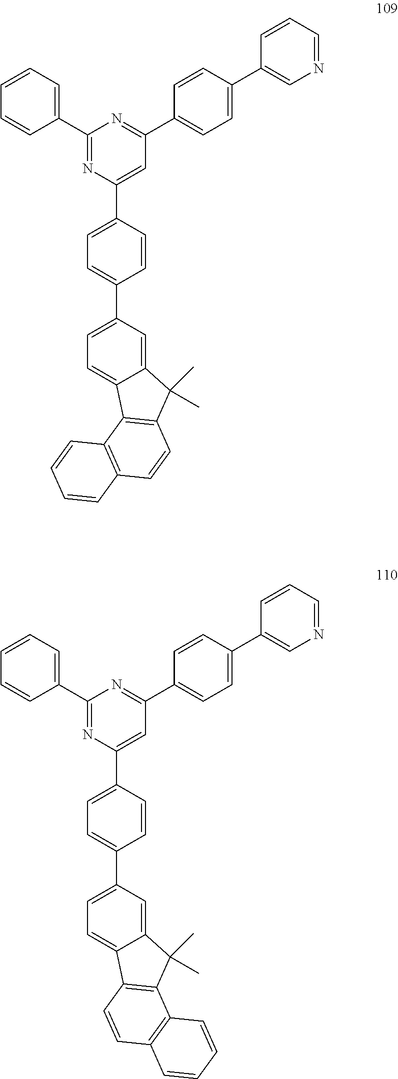 [SMILES: CC1(C)c2cc(-c3ccc(-c4cc(-c5ccc(-c6cccnc6)cc5)nc(-c5ccccc5)n4)cc3)ccc2-c2c1ccc1ccccc21 ; CC1(C)c2cc(-c3ccc(-c4cc(-c5ccc(-c6cccnc6)cc5)nc(-c5ccccc5)n4)cc3)ccc2-c2ccc3ccccc3c21]

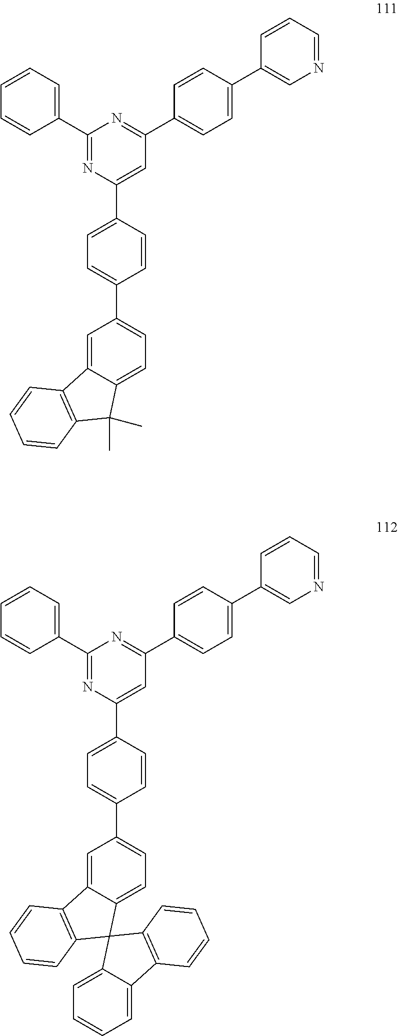 [SMILES: CC1(C)c2ccccc2-c2cc(-c3ccc(-c4cc(-c5ccc(-c6cccnc6)cc5)nc(-c5ccccc5)n4)cc3)ccc21 ; c1ccc(-c2nc(-c3ccc(-c4cccnc4)cc3)cc(-c3ccc(-c4ccc5c(c4)-c4ccccc4C54c5ccccc5-c5ccccc54)cc3)n2)cc1]

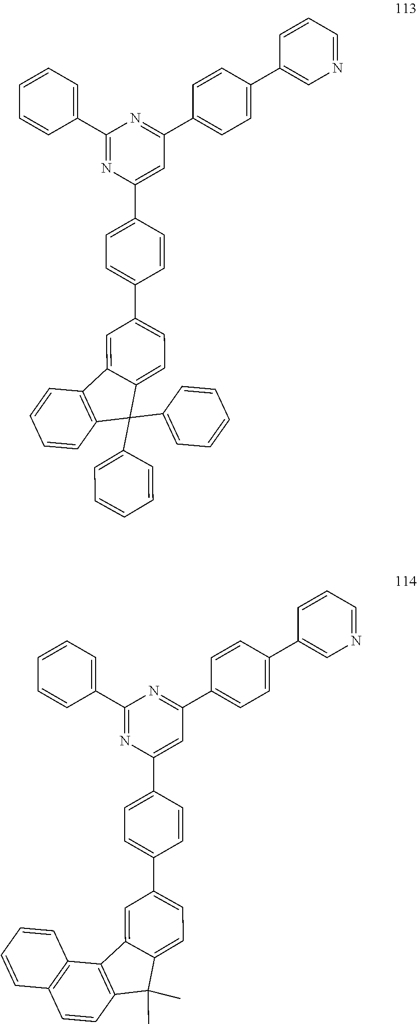 [SMILES: c1ccc(-c2nc(-c3ccc(-c4cccnc4)cc3)cc(-c3ccc(-c4ccc5c(c4)-c4ccccc4C5(c4ccccc4)c4ccccc4)cc3)n2)cc1 ; CC1(C)c2ccc(-c3ccc(-c4cc(-c5ccc(-c6cccnc6)cc5)nc(-c5ccccc5)n4)cc3)cc2-c2c1ccc1ccccc21]

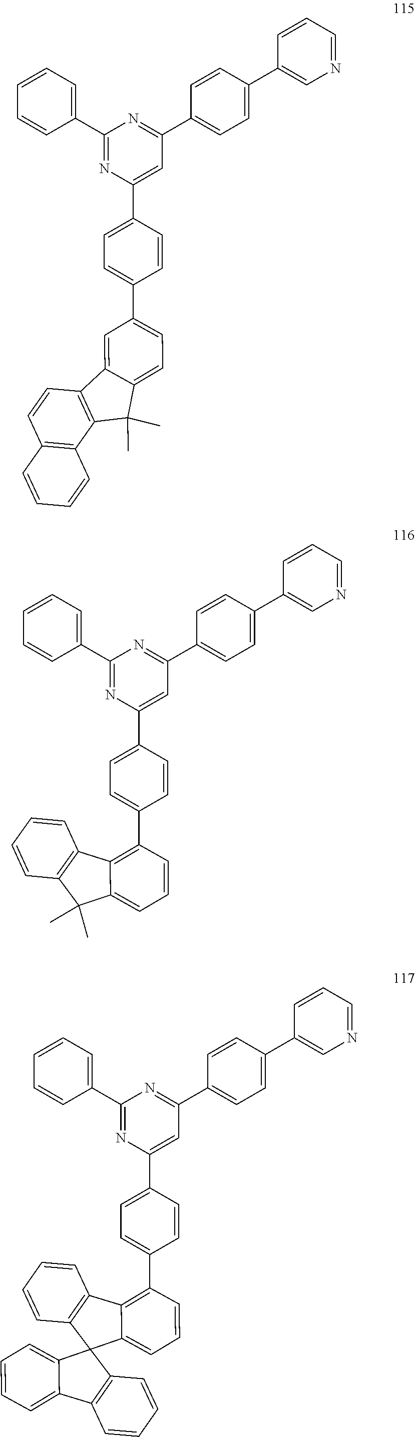 [SMILES: CC1(C)c2ccc(-c3ccc(-c4cc(-c5ccc(-c6cccnc6)cc5)nc(-c5ccccc5)n4)cc3)cc2-c2ccc3ccccc3c21 ; CC1(C)c2ccccc2-c2c(-c3ccc(-c4cc(-c5ccc(-c6cccnc6)cc5)nc(-c5ccccc5)n4)cc3)cccc21 ; c1ccc(-c2nc(-c3ccc(-c4cccnc4)cc3)cc(-c3ccc(-c4cccc5c4-c4ccccc4C54c5ccccc5-c5ccccc54)cc3)n2)cc1]

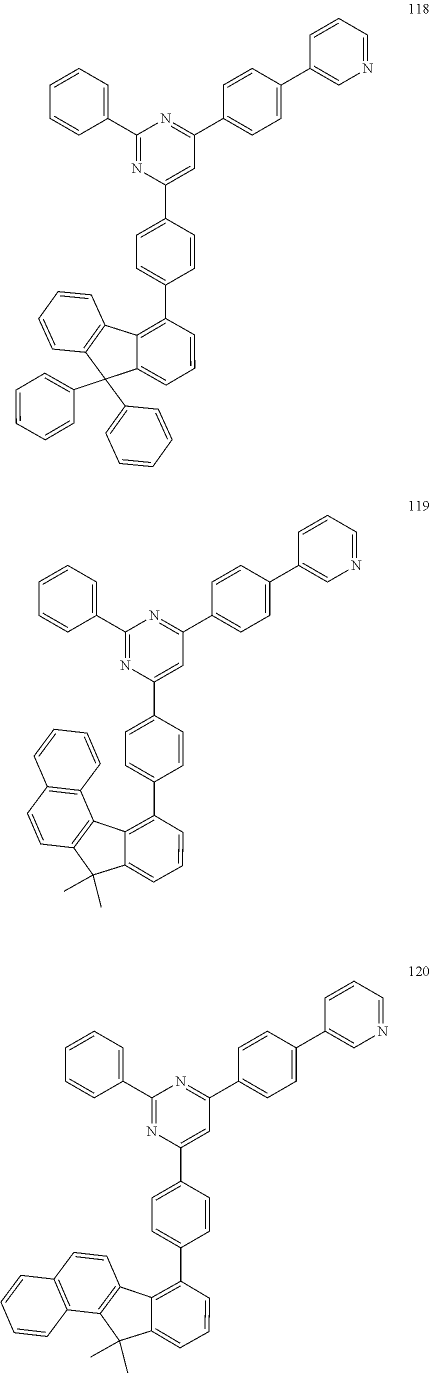 [SMILES: c1ccc(-c2nc(-c3ccc(-c4cccnc4)cc3)cc(-c3ccc(-c4cccc5c4-c4ccccc4C5(c4ccccc4)c4ccccc4)cc3)n2)cc1 ; CC1(C)c2cccc(-c3ccc(-c4cc(-c5ccc(-c6cccnc6)cc5)nc(-c5ccccc5)n4)cc3)c2-c2c1ccc1ccccc21 ; CC1(C)c2cccc(-c3ccc(-c4cc(-c5ccc(-c6cccnc6)cc5)nc(-c5ccccc5)n4)cc3)c2-c2ccc3ccccc3c21]

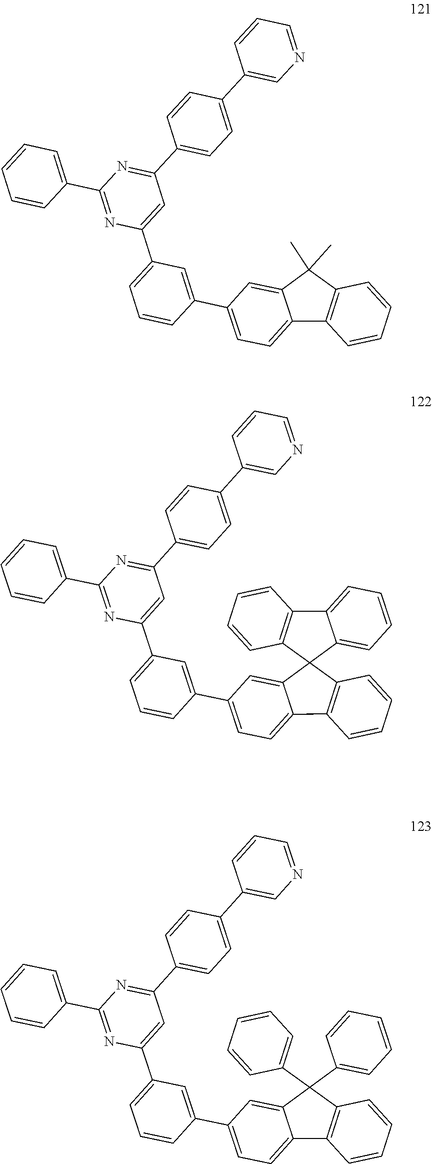 [SMILES: CC1(C)c2ccccc2-c2ccc(-c3cccc(-c4cc(-c5ccc(-c6cccnc6)cc5)nc(-c5ccccc5)n4)c3)cc21 ; c1ccc(-c2nc(-c3ccc(-c4cccnc4)cc3)cc(-c3cccc(-c4ccc5c(c4)C4(c6ccccc6-c6ccccc64)c4ccccc4-5)c3)n2)cc1 ; c1ccc(-c2nc(-c3ccc(-c4cccnc4)cc3)cc(-c3cccc(-c4ccc5c(c4)C(c4ccccc4)(c4ccccc4)c4ccccc4-5)c3)n2)cc1]

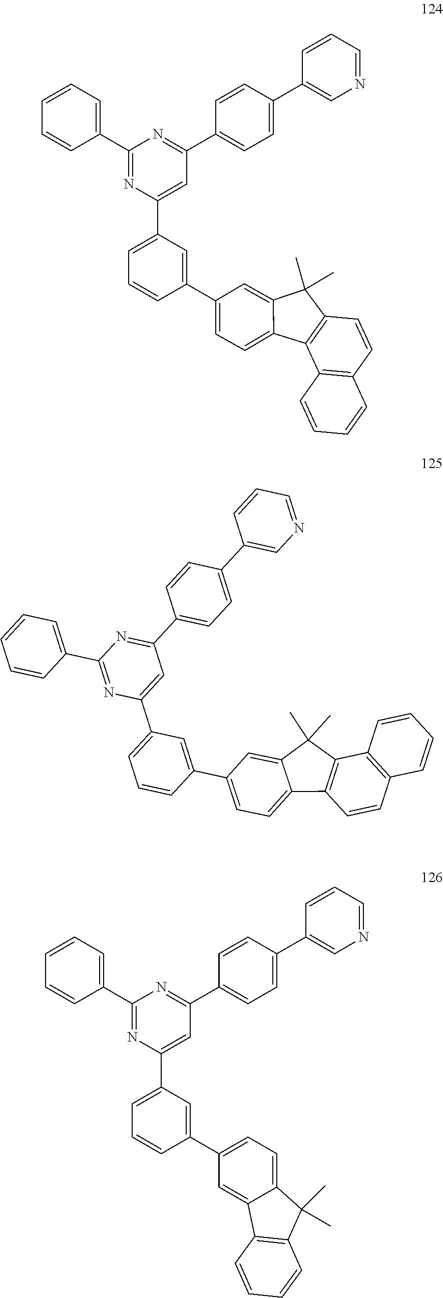 [SMILES: CC1(C)c2cc(-c3cccc(-c4cc(-c5ccc(-c6cccnc6)cc5)nc(-c5ccccc5)n4)c3)ccc2-c2c1ccc1ccccc21 ; CC1(C)c2cc(-c3cccc(-c4cc(-c5ccc(-c6cccnc6)cc5)nc(-c5ccccc5)n4)c3)ccc2-c2ccc3ccccc3c21 ; CC1(C)c2ccccc2-c2cc(-c3cccc(-c4cc(-c5ccc(-c6cccnc6)cc5)nc(-c5ccccc5)n4)c3)ccc21]

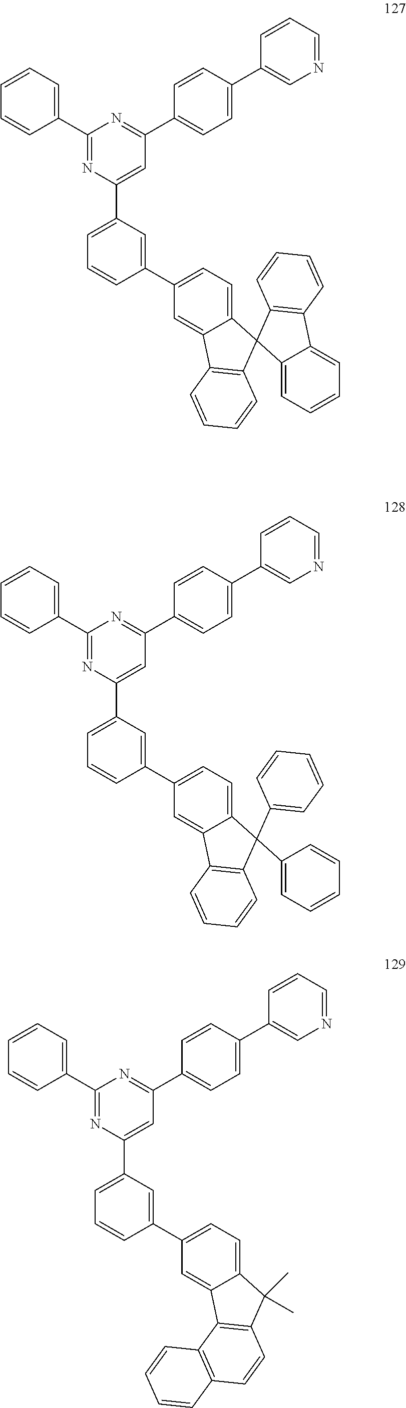 [SMILES: c1ccc(-c2nc(-c3ccc(-c4cccnc4)cc3)cc(-c3cccc(-c4ccc5c(c4)-c4ccccc4C54c5ccccc5-c5ccccc54)c3)n2)cc1 ; c1ccc(-c2nc(-c3ccc(-c4cccnc4)cc3)cc(-c3cccc(-c4ccc5c(c4)-c4ccccc4C5(c4ccccc4)c4ccccc4)c3)n2)cc1 ; CC1(C)c2ccc(-c3cccc(-c4cc(-c5ccc(-c6cccnc6)cc5)nc(-c5ccccc5)n4)c3)cc2-c2c1ccc1ccccc21]

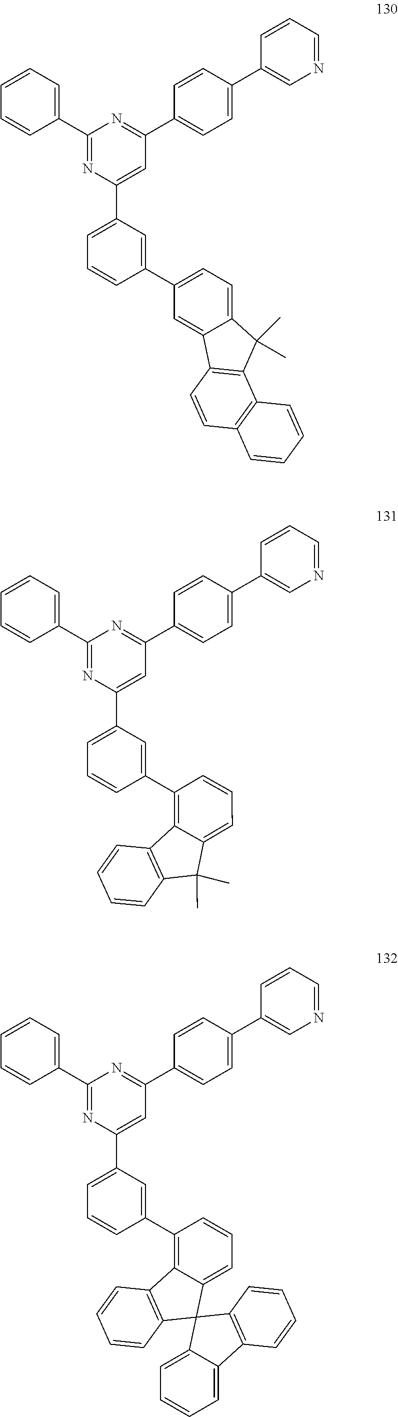 [SMILES: CC1(C)c2ccc(-c3cccc(-c4cc(-c5ccc(-c6cccnc6)cc5)nc(-c5ccccc5)n4)c3)cc2-c2ccc3ccccc3c21 ; CC1(C)c2ccccc2-c2c(-c3cccc(-c4cc(-c5ccc(-c6cccnc6)cc5)nc(-c5ccccc5)n4)c3)cccc21 ; c1ccc(-c2nc(-c3ccc(-c4cccnc4)cc3)cc(-c3cccc(-c4cccc5c4-c4ccccc4C54c5ccccc5-c5ccccc54)c3)n2)cc1]

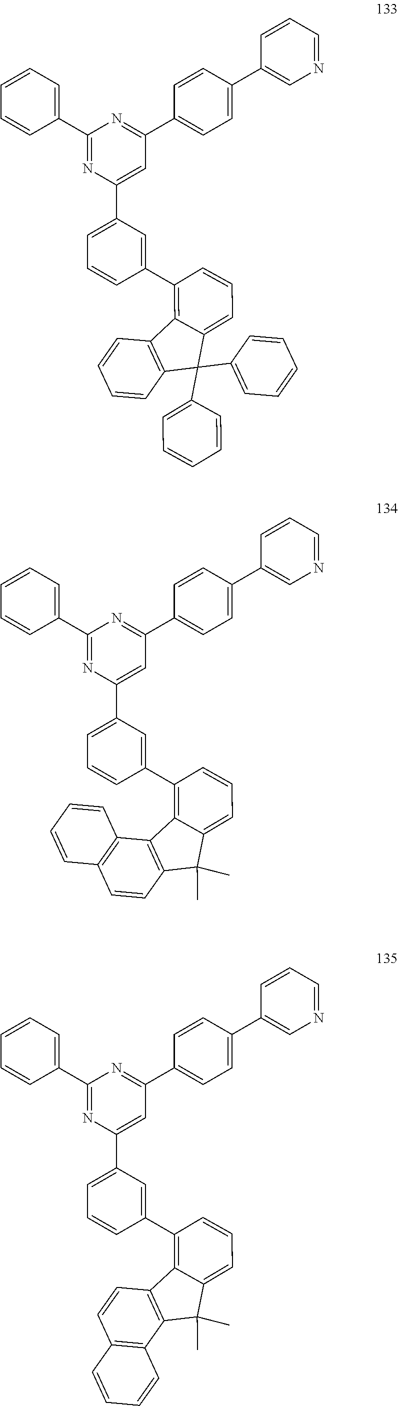 [SMILES: c1ccc(-c2nc(-c3ccc(-c4cccnc4)cc3)cc(-c3cccc(-c4cccc5c4-c4ccccc4C5(c4ccccc4)c4ccccc4)c3)n2)cc1 ; CC1(C)c2cccc(-c3cccc(-c4cc(-c5ccc(-c6cccnc6)cc5)nc(-c5ccccc5)n4)c3)c2-c2c1ccc1ccccc21 ; CC1(C)c2cccc(-c3cccc(-c4cc(-c5ccc(-c6cccnc6)cc5)nc(-c5ccccc5)n4)c3)c2-c2ccc3ccccc3c21]

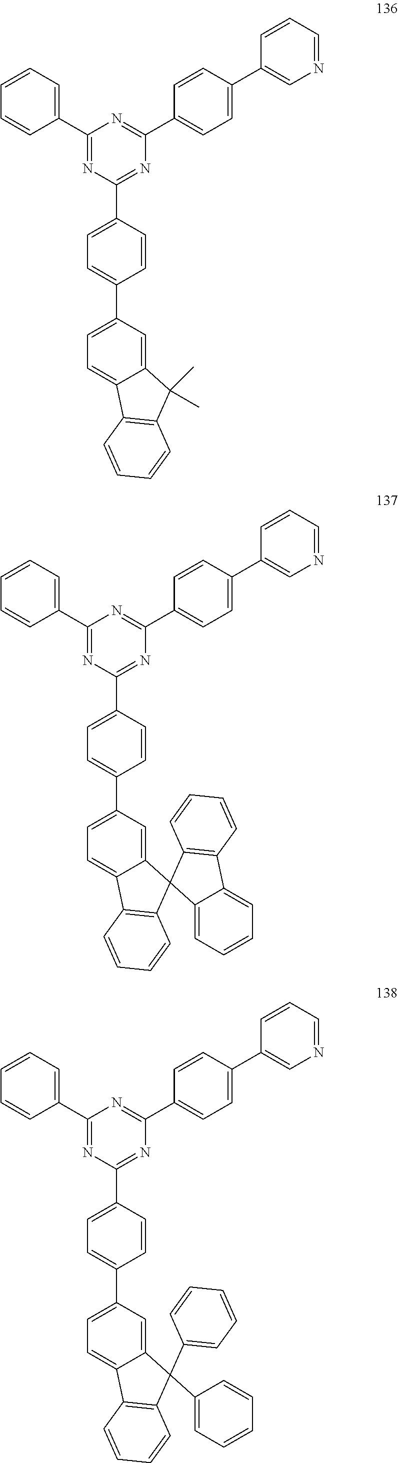 [SMILES: CC1(C)c2ccccc2-c2ccc(-c3ccc(-c4nc(-c5ccccc5)nc(-c5ccc(-c6cccnc6)cc5)n4)cc3)cc21 ; c1ccc(-c2nc(-c3ccc(-c4cccnc4)cc3)nc(-c3ccc(-c4ccc5c(c4)C4(c6ccccc6-c6ccccc64)c4ccccc4-5)cc3)n2)cc1 ; c1ccc(-c2nc(-c3ccc(-c4cccnc4)cc3)nc(-c3ccc(-c4ccc5c(c4)C(c4ccccc4)(c4ccccc4)c4ccccc4-5)cc3)n2)cc1]

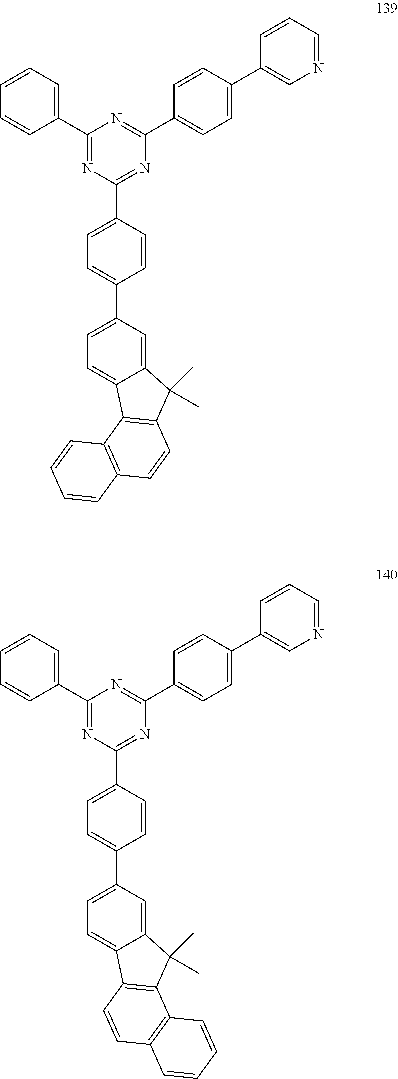 [SMILES: CC1(C)c2cc(-c3ccc(-c4nc(-c5ccccc5)nc(-c5ccc(-c6cccnc6)cc5)n4)cc3)ccc2-c2c1ccc1ccccc21 ; CC1(C)c2cc(-c3ccc(-c4nc(-c5ccccc5)nc(-c5ccc(-c6cccnc6)cc5)n4)cc3)ccc2-c2ccc3ccccc3c21]

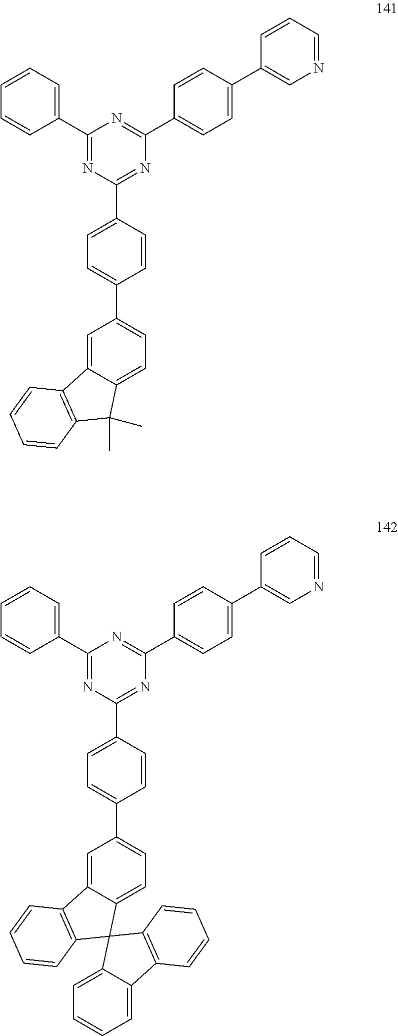 [SMILES: CC1(C)c2ccccc2-c2cc(-c3ccc(-c4nc(-c5ccccc5)nc(-c5ccc(-c6cccnc6)cc5)n4)cc3)ccc21 ; c1ccc(-c2nc(-c3ccc(-c4cccnc4)cc3)nc(-c3ccc(-c4ccc5c(c4)-c4ccccc4C54c5ccccc5-c5ccccc54)cc3)n2)cc1]

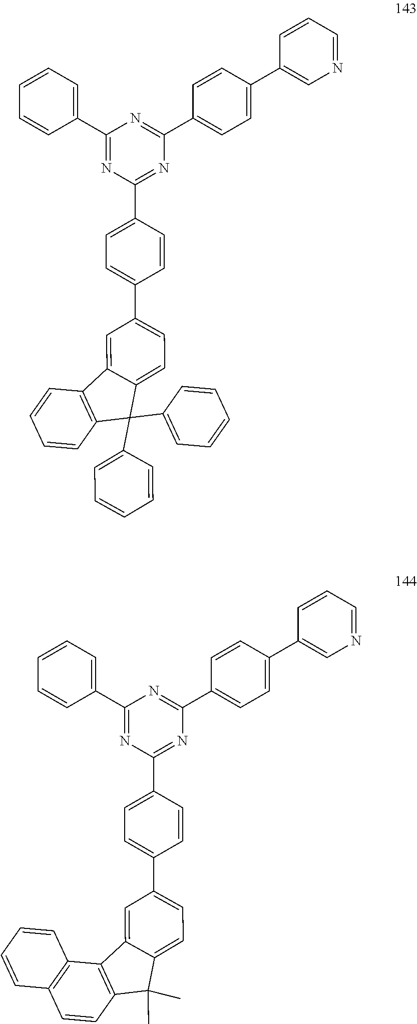 [SMILES: c1ccc(-c2nc(-c3ccc(-c4cccnc4)cc3)nc(-c3ccc(-c4ccc5c(c4)-c4ccccc4C5(c4ccccc4)c4ccccc4)cc3)n2)cc1 ; CC1(C)c2ccc(-c3ccc(-c4nc(-c5ccccc5)nc(-c5ccc(-c6cccnc6)cc5)n4)cc3)cc2-c2c1ccc1ccccc21]

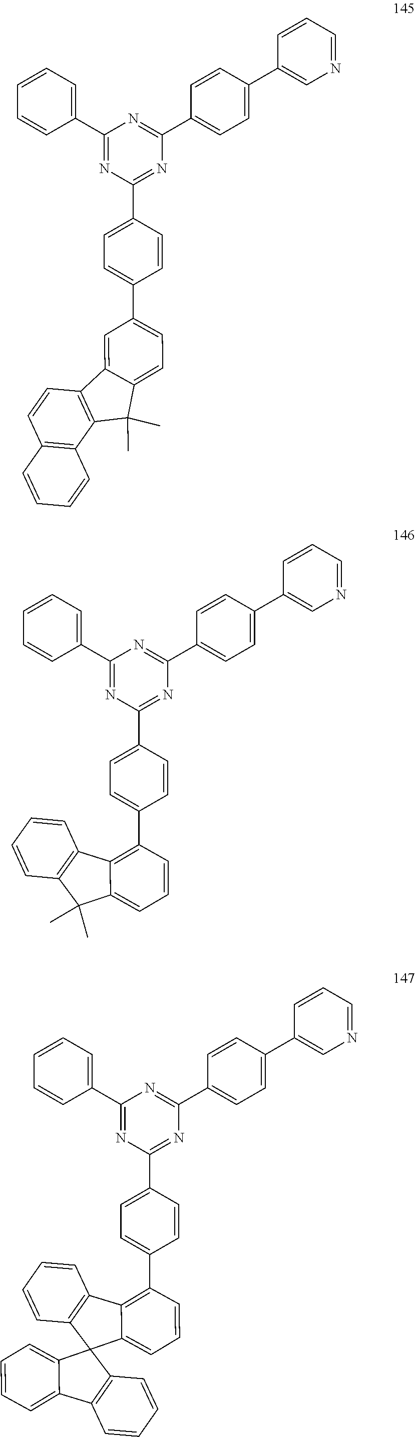 [SMILES: CC1(C)c2ccc(-c3ccc(-c4nc(-c5ccccc5)nc(-c5ccc(-c6cccnc6)cc5)n4)cc3)cc2-c2ccc3ccccc3c21 ; CC1(C)c2ccccc2-c2c(-c3ccc(-c4nc(-c5ccccc5)nc(-c5ccc(-c6cccnc6)cc5)n4)cc3)cccc21 ; c1ccc(-c2nc(-c3ccc(-c4cccnc4)cc3)nc(-c3ccc(-c4cccc5c4-c4ccccc4C54c5ccccc5-c5ccccc54)cc3)n2)cc1]

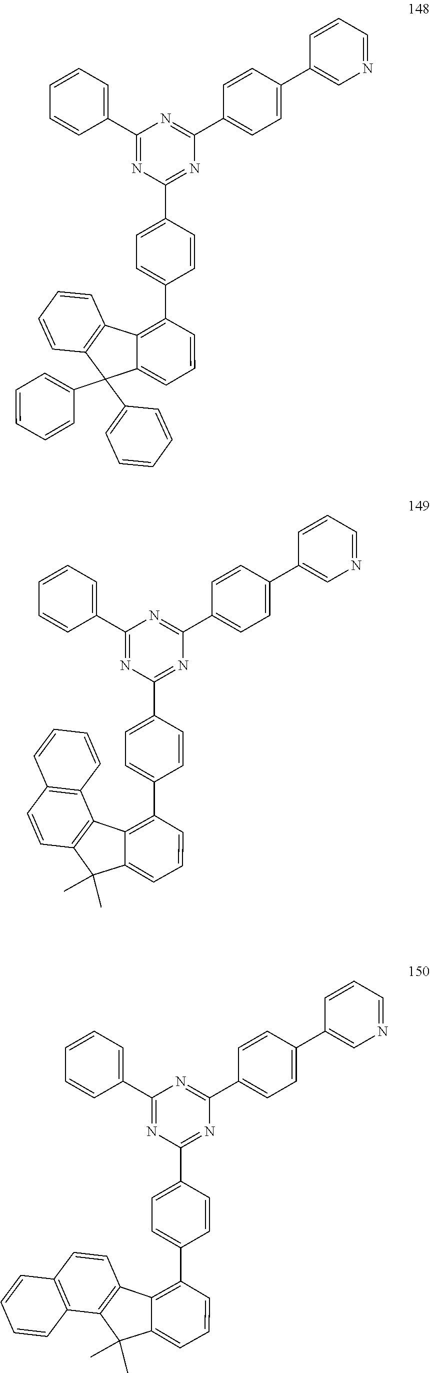 [SMILES: c1ccc(-c2nc(-c3ccc(-c4cccnc4)cc3)nc(-c3ccc(-c4cccc5c4-c4ccccc4C5(c4ccccc4)c4ccccc4)cc3)n2)cc1 ; CC1(C)c2cccc(-c3ccc(-c4nc(-c5ccccc5)nc(-c5ccc(-c6cccnc6)cc5)n4)cc3)c2-c2c1ccc1ccccc21 ; CC1(C)c2cccc(-c3ccc(-c4nc(-c5ccccc5)nc(-c5ccc(-c6cccnc6)cc5)n4)cc3)c2-c2ccc3ccccc3c21]

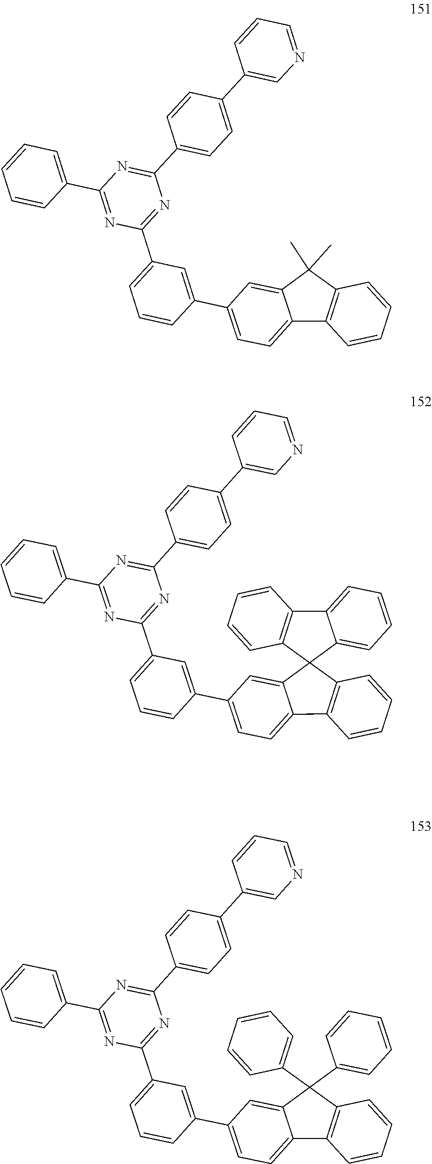 [SMILES: CC1(C)c2ccccc2-c2ccc(-c3cccc(-c4nc(-c5ccccc5)nc(-c5ccc(-c6cccnc6)cc5)n4)c3)cc21 ; c1ccc(-c2nc(-c3ccc(-c4cccnc4)cc3)nc(-c3cccc(-c4ccc5c(c4)C4(c6ccccc6-c6ccccc64)c4ccccc4-5)c3)n2)cc1 ; c1ccc(-c2nc(-c3ccc(-c4cccnc4)cc3)nc(-c3cccc(-c4ccc5c(c4)C(c4ccccc4)(c4ccccc4)c4ccccc4-5)c3)n2)cc1]

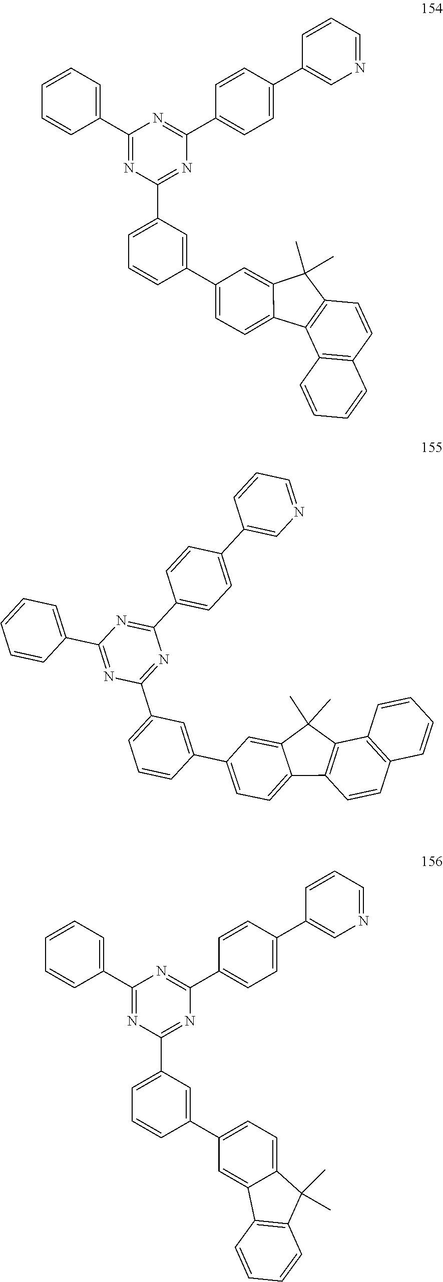 [SMILES: CC1(C)c2cc(-c3cccc(-c4nc(-c5ccccc5)nc(-c5ccc(-c6cccnc6)cc5)n4)c3)ccc2-c2c1ccc1ccccc21 ; CC1(C)c2cc(-c3cccc(-c4nc(-c5ccccc5)nc(-c5ccc(-c6cccnc6)cc5)n4)c3)ccc2-c2ccc3ccccc3c21 ; CC1(C)c2ccccc2-c2cc(-c3cccc(-c4nc(-c5ccccc5)nc(-c5ccc(-c6cccnc6)cc5)n4)c3)ccc21]

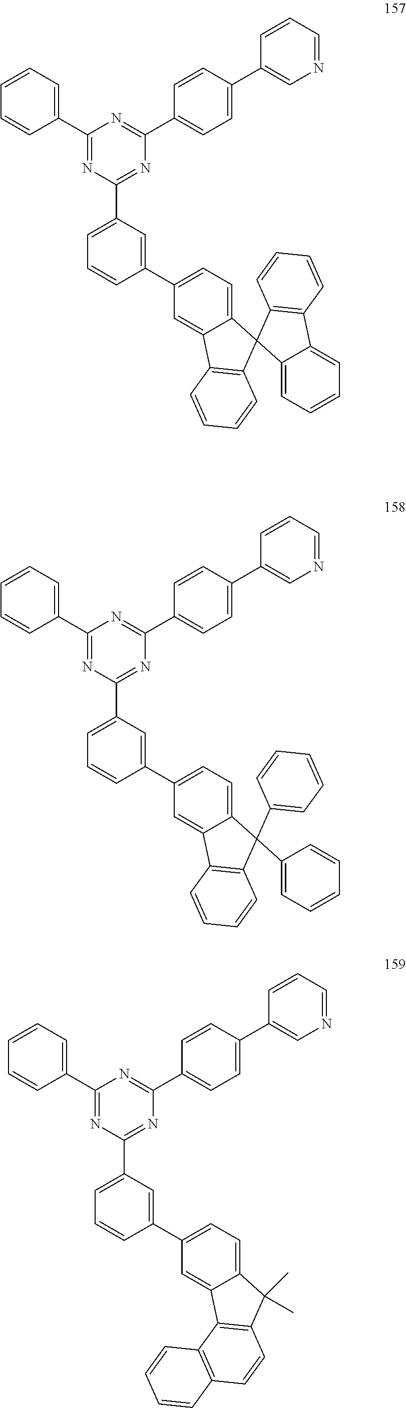 [SMILES: c1ccc(-c2nc(-c3ccc(-c4cccnc4)cc3)nc(-c3cccc(-c4ccc5c(c4)-c4ccccc4C54c5ccccc5-c5ccccc54)c3)n2)cc1 ; c1ccc(-c2nc(-c3ccc(-c4cccnc4)cc3)nc(-c3cccc(-c4ccc5c(c4)-c4ccccc4C5(c4ccccc4)c4ccccc4)c3)n2)cc1 ; CC1(C)c2ccc(-c3cccc(-c4nc(-c5ccccc5)nc(-c5ccc(-c6cccnc6)cc5)n4)c3)cc2-c2c1ccc1ccccc21]

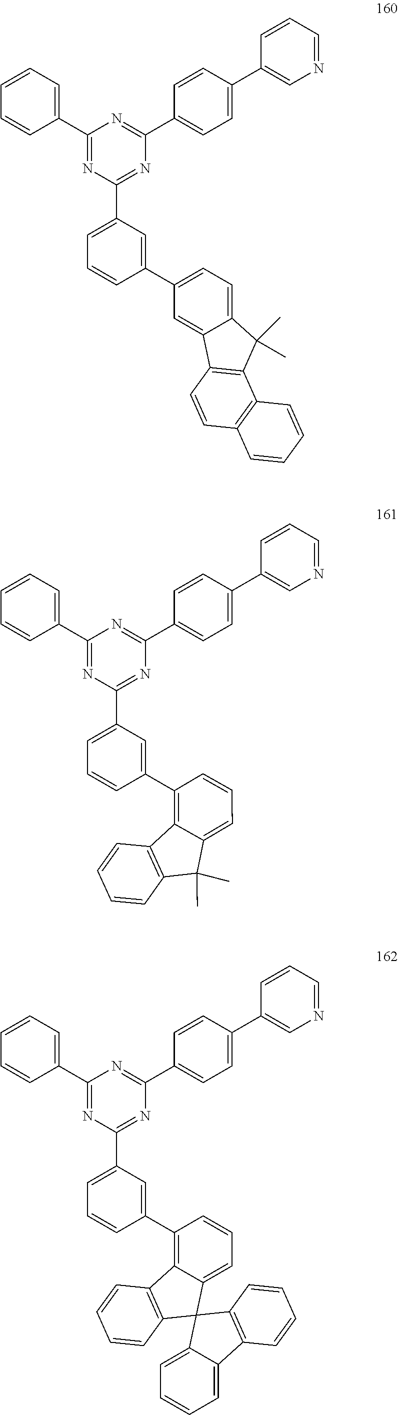 [SMILES: CC1(C)c2ccc(-c3cccc(-c4nc(-c5ccccc5)nc(-c5ccc(-c6cccnc6)cc5)n4)c3)cc2-c2ccc3ccccc3c21 ; CC1(C)c2ccccc2-c2c(-c3cccc(-c4nc(-c5ccccc5)nc(-c5ccc(-c6cccnc6)cc5)n4)c3)cccc21 ; c1ccc(-c2nc(-c3ccc(-c4cccnc4)cc3)nc(-c3cccc(-c4cccc5c4-c4ccccc4C54c5ccccc5-c5ccccc54)c3)n2)cc1]

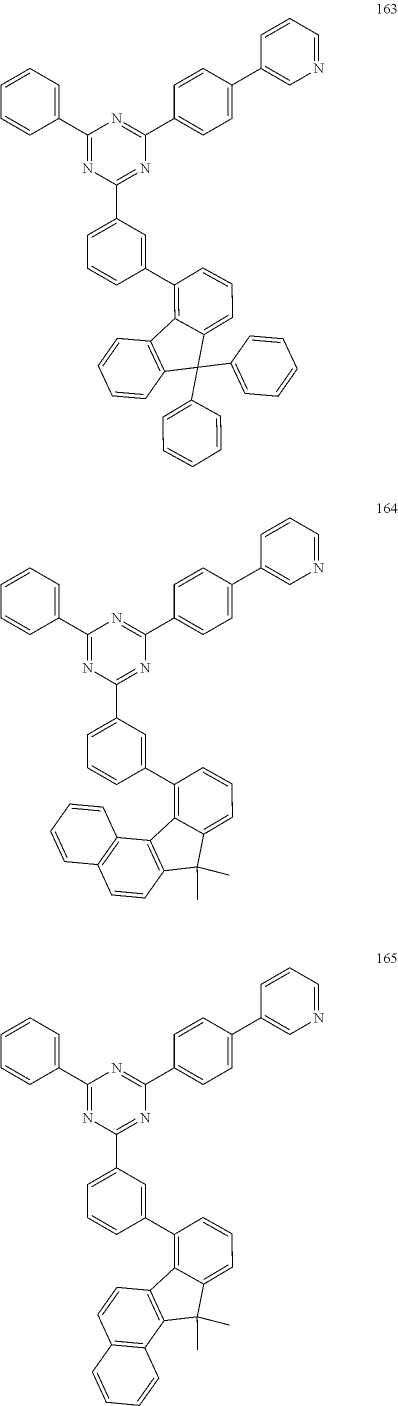 [SMILES: c1ccc(-c2nc(-c3ccc(-c4cccnc4)cc3)nc(-c3cccc(-c4cccc5c4-c4ccccc4C5(c4ccccc4)c4ccccc4)c3)n2)cc1 ; CC1(C)c2cccc(-c3cccc(-c4nc(-c5ccccc5)nc(-c5ccc(-c6cccnc6)cc5)n4)c3)c2-c2c1ccc1ccccc21 ; CC1(C)c2cccc(-c3cccc(-c4nc(-c5ccccc5)nc(-c5ccc(-c6cccnc6)cc5)n4)c3)c2-c2ccc3ccccc3c21]

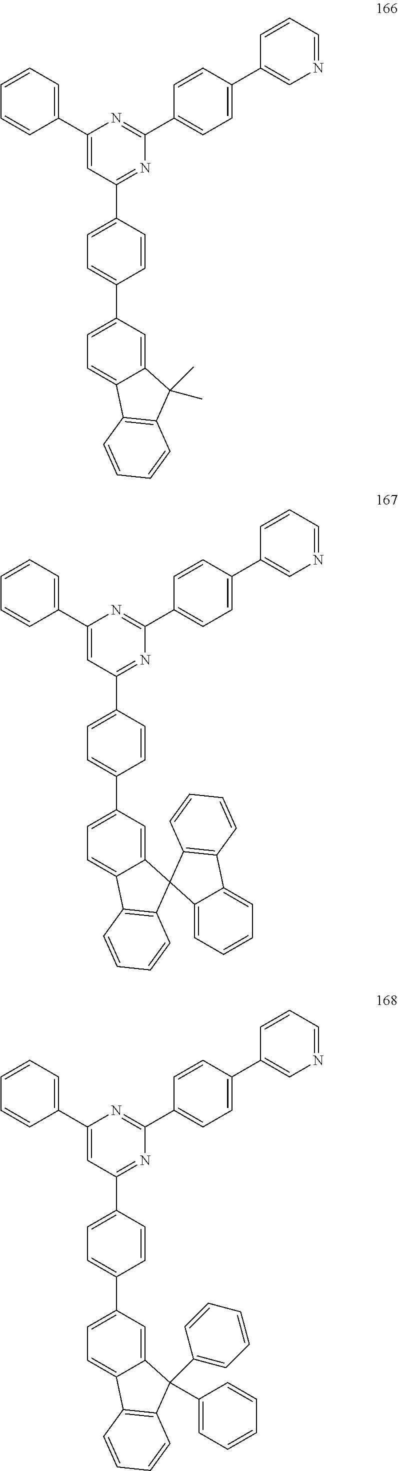 [SMILES: CC1(C)c2ccccc2-c2ccc(-c3ccc(-c4cc(-c5ccccc5)nc(-c5ccc(-c6cccnc6)cc5)n4)cc3)cc21 ; c1ccc(-c2cc(-c3ccc(-c4ccc5c(c4)C4(c6ccccc6-c6ccccc64)c4ccccc4-5)cc3)nc(-c3ccc(-c4cccnc4)cc3)n2)cc1 ; c1ccc(-c2cc(-c3ccc(-c4ccc5c(c4)C(c4ccccc4)(c4ccccc4)c4ccccc4-5)cc3)nc(-c3ccc(-c4cccnc4)cc3)n2)cc1]

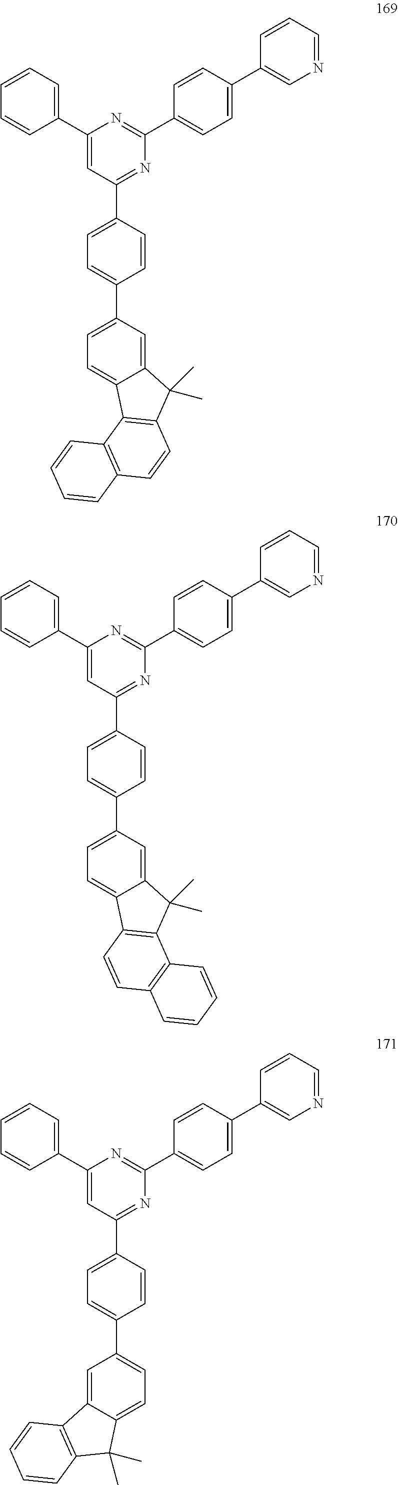 [SMILES: CC1(C)c2cc(-c3ccc(-c4cc(-c5ccccc5)nc(-c5ccc(-c6cccnc6)cc5)n4)cc3)ccc2-c2c1ccc1ccccc21 ; CC1(C)c2cc(-c3ccc(-c4cc(-c5ccccc5)nc(-c5ccc(-c6cccnc6)cc5)n4)cc3)ccc2-c2ccc3ccccc3c21 ; CC1(C)c2ccccc2-c2cc(-c3ccc(-c4cc(-c5ccccc5)nc(-c5ccc(-c6cccnc6)cc5)n4)cc3)ccc21]

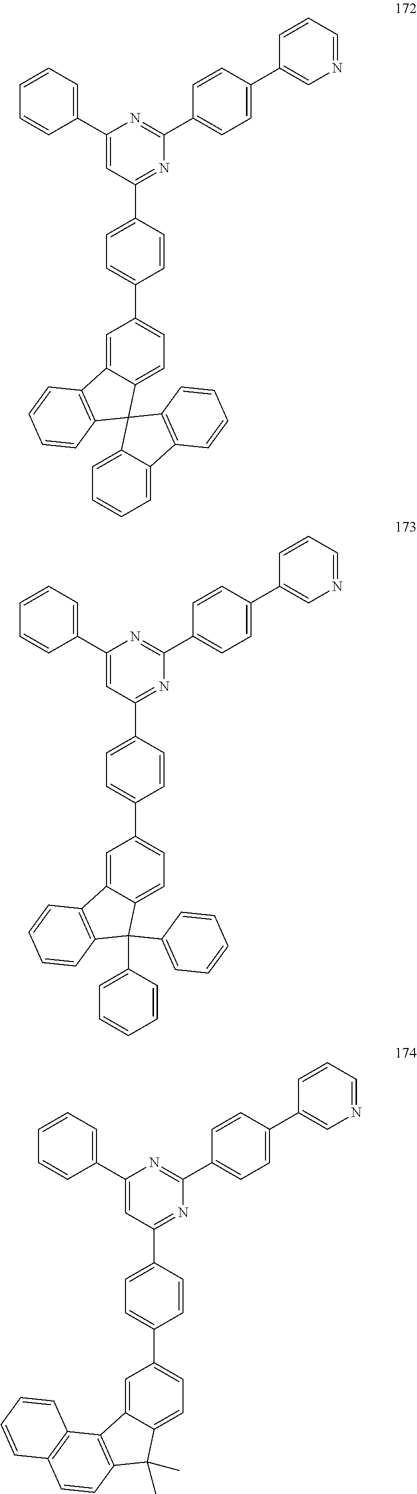 [SMILES: c1ccc(-c2cc(-c3ccc(-c4ccc5c(c4)-c4ccccc4C54c5ccccc5-c5ccccc54)cc3)nc(-c3ccc(-c4cccnc4)cc3)n2)cc1 ; c1ccc(-c2cc(-c3ccc(-c4ccc5c(c4)-c4ccccc4C5(c4ccccc4)c4ccccc4)cc3)nc(-c3ccc(-c4cccnc4)cc3)n2)cc1 ; CC1(C)c2ccc(-c3ccc(-c4cc(-c5ccccc5)nc(-c5ccc(-c6cccnc6)cc5)n4)cc3)cc2-c2c1ccc1ccccc21]

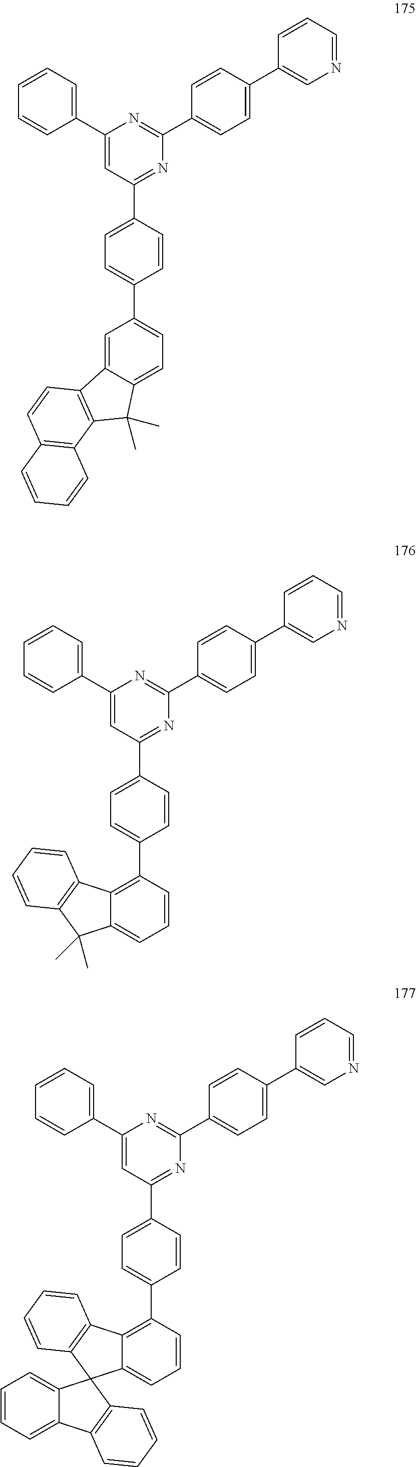 [SMILES: CC1(C)c2ccc(-c3ccc(-c4cc(-c5ccccc5)nc(-c5ccc(-c6cccnc6)cc5)n4)cc3)cc2-c2ccc3ccccc3c21 ; CC1(C)c2ccccc2-c2c(-c3ccc(-c4cc(-c5ccccc5)nc(-c5ccc(-c6cccnc6)cc5)n4)cc3)cccc21 ; c1ccc(-c2cc(-c3ccc(-c4cccc5c4-c4ccccc4C54c5ccccc5-c5ccccc54)cc3)nc(-c3ccc(-c4cccnc4)cc3)n2)cc1]

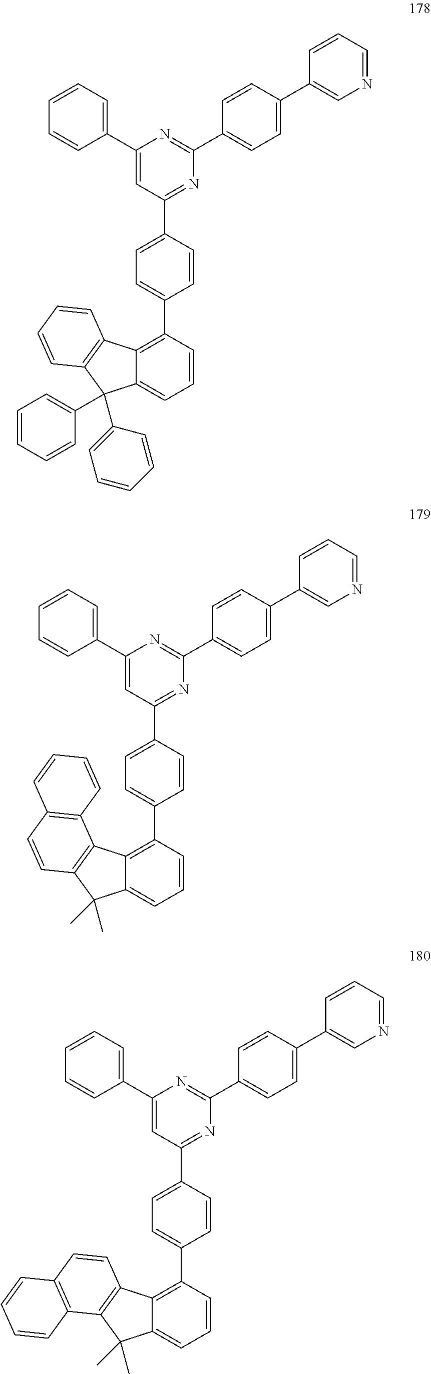 [SMILES: c1ccc(-c2cc(-c3ccc(-c4cccc5c4-c4ccccc4C5(c4ccccc4)c4ccccc4)cc3)nc(-c3ccc(-c4cccnc4)cc3)n2)cc1 ; CC1(C)c2cccc(-c3ccc(-c4cc(-c5ccccc5)nc(-c5ccc(-c6cccnc6)cc5)n4)cc3)c2-c2c1ccc1ccccc21 ; CC1(C)c2cccc(-c3ccc(-c4cc(-c5ccccc5)nc(-c5ccc(-c6cccnc6)cc5)n4)cc3)c2-c2ccc3ccccc3c21]

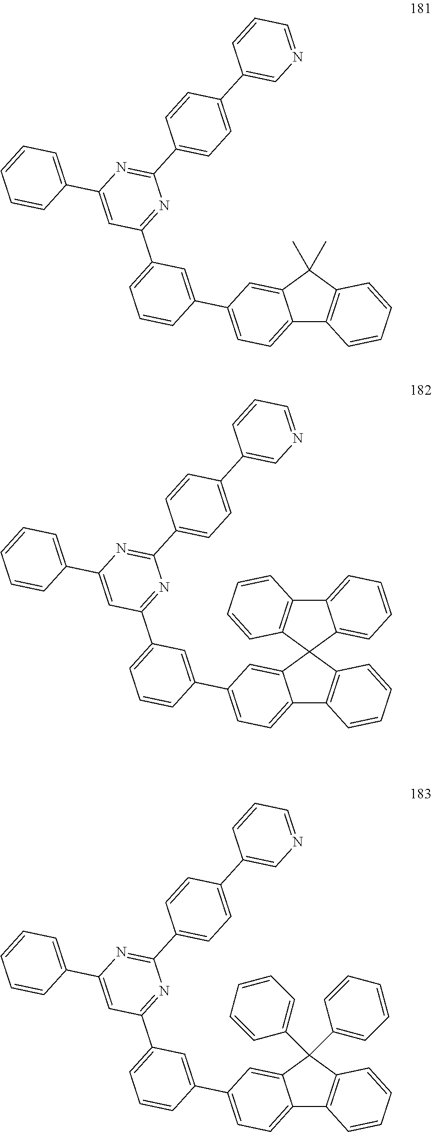 [SMILES: CC1(C)c2ccccc2-c2ccc(-c3cccc(-c4cc(-c5ccccc5)nc(-c5ccc(-c6cccnc6)cc5)n4)c3)cc21 ; c1ccc(-c2cc(-c3cccc(-c4ccc5c(c4)C4(c6ccccc6-c6ccccc64)c4ccccc4-5)c3)nc(-c3ccc(-c4cccnc4)cc3)n2)cc1 ; c1ccc(-c2cc(-c3cccc(-c4ccc5c(c4)C(c4ccccc4)(c4ccccc4)c4ccccc4-5)c3)nc(-c3ccc(-c4cccnc4)cc3)n2)cc1]

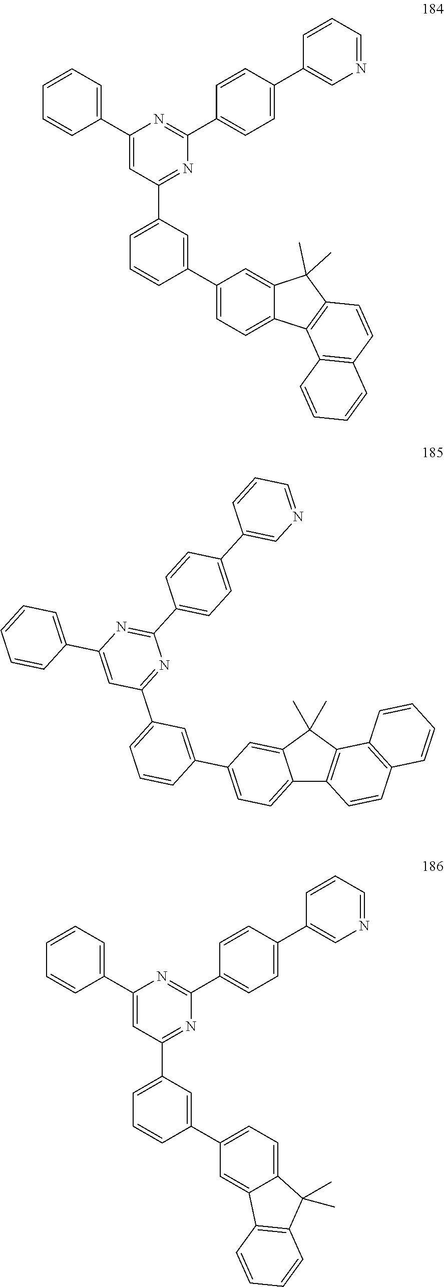 [SMILES: CC1(C)c2cc(-c3cccc(-c4cc(-c5ccccc5)nc(-c5ccc(-c6cccnc6)cc5)n4)c3)ccc2-c2c1ccc1ccccc21 ; CC1(C)c2cc(-c3cccc(-c4cc(-c5ccccc5)nc(-c5ccc(-c6cccnc6)cc5)n4)c3)ccc2-c2ccc3ccccc3c21 ; CC1(C)c2ccccc2-c2cc(-c3cccc(-c4cc(-c5ccccc5)nc(-c5ccc(-c6cccnc6)cc5)n4)c3)ccc21]

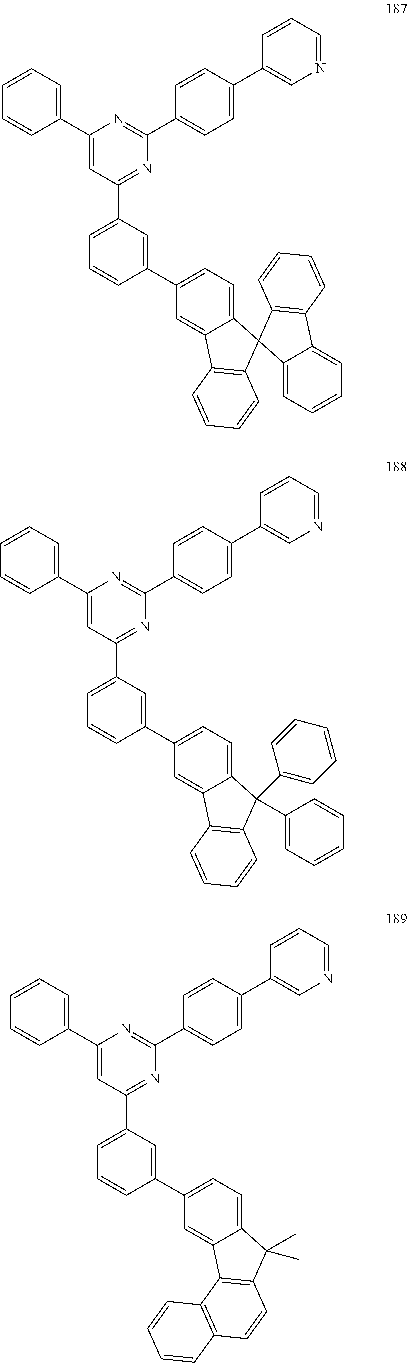 [SMILES: c1ccc(-c2cc(-c3cccc(-c4ccc5c(c4)-c4ccccc4C54c5ccccc5-c5ccccc54)c3)nc(-c3ccc(-c4cccnc4)cc3)n2)cc1 ; c1ccc(-c2cc(-c3cccc(-c4ccc5c(c4)-c4ccccc4C5(c4ccccc4)c4ccccc4)c3)nc(-c3ccc(-c4cccnc4)cc3)n2)cc1 ; CC1(C)c2ccc(-c3cccc(-c4cc(-c5ccccc5)nc(-c5ccc(-c6cccnc6)cc5)n4)c3)cc2-c2c1ccc1ccccc21]

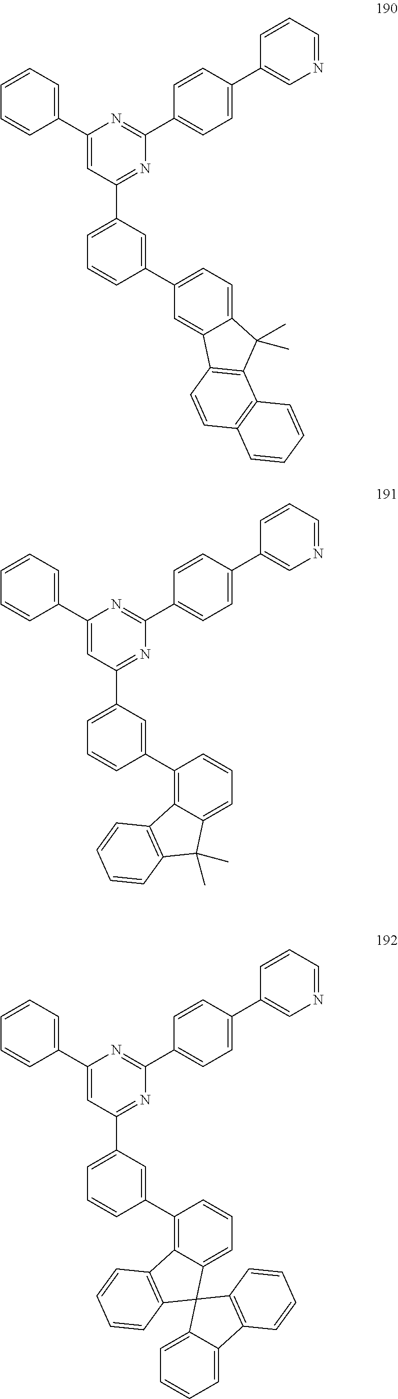

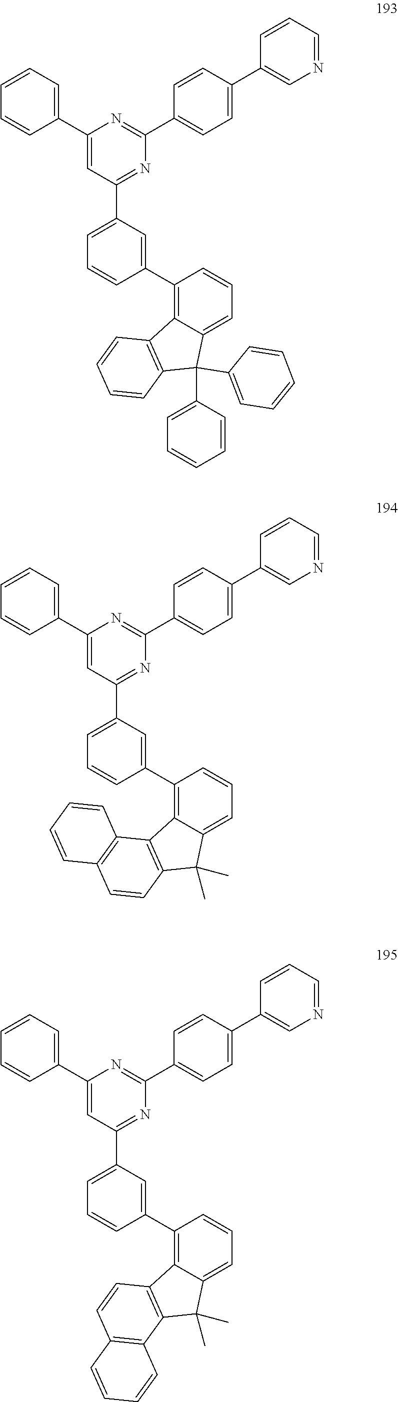 [SMILES: c1ccc(-c2cc(-c3cccc(-c4cccc5c4-c4ccccc4C5(c4ccccc4)c4ccccc4)c3)nc(-c3ccc(-c4cccnc4)cc3)n2)cc1 ; CC1(C)c2cccc(-c3cccc(-c4cc(-c5ccccc5)nc(-c5ccc(-c6cccnc6)cc5)n4)c3)c2-c2c1ccc1ccccc21 ; CC1(C)c2cccc(-c3cccc(-c4cc(-c5ccccc5)nc(-c5ccc(-c6cccnc6)cc5)n4)c3)c2-c2ccc3ccccc3c21]

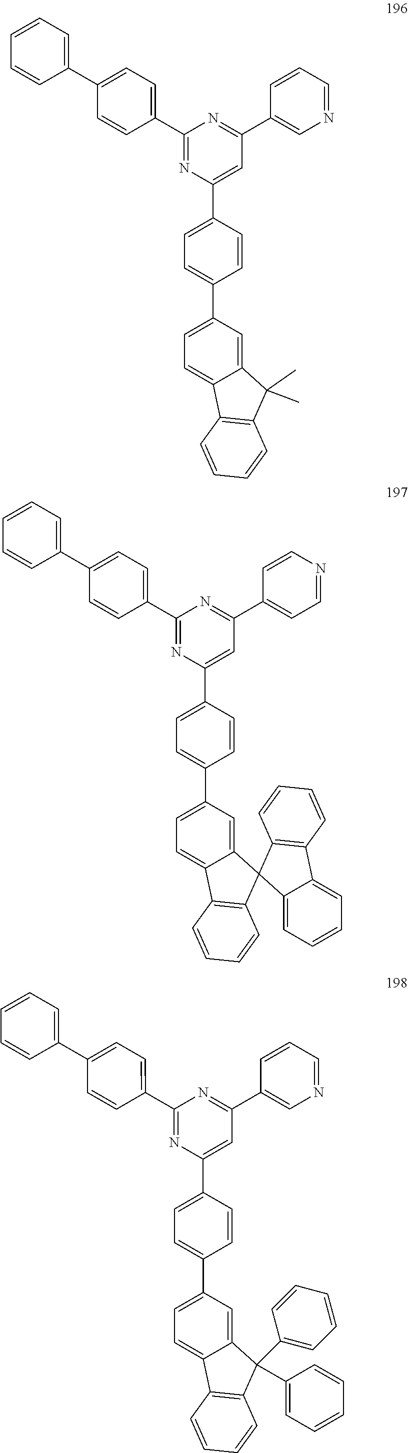 [SMILES: CC1(C)c2ccccc2-c2ccc(-c3ccc(-c4cc(-c5cccnc5)nc(-c5ccc(-c6ccccc6)cc5)n4)cc3)cc21 ; c1ccc(-c2ccc(-c3nc(-c4ccncc4)cc(-c4ccc(-c5ccc6c(c5)C5(c7ccccc7-c7ccccc75)c5ccccc5-6)cc4)n3)cc2)cc1 ; c1ccc(-c2ccc(-c3nc(-c4ccc(-c5ccc6c(c5)C(c5ccccc5)(c5ccccc5)c5ccccc5-6)cc4)cc(-c4cccnc4)n3)cc2)cc1]

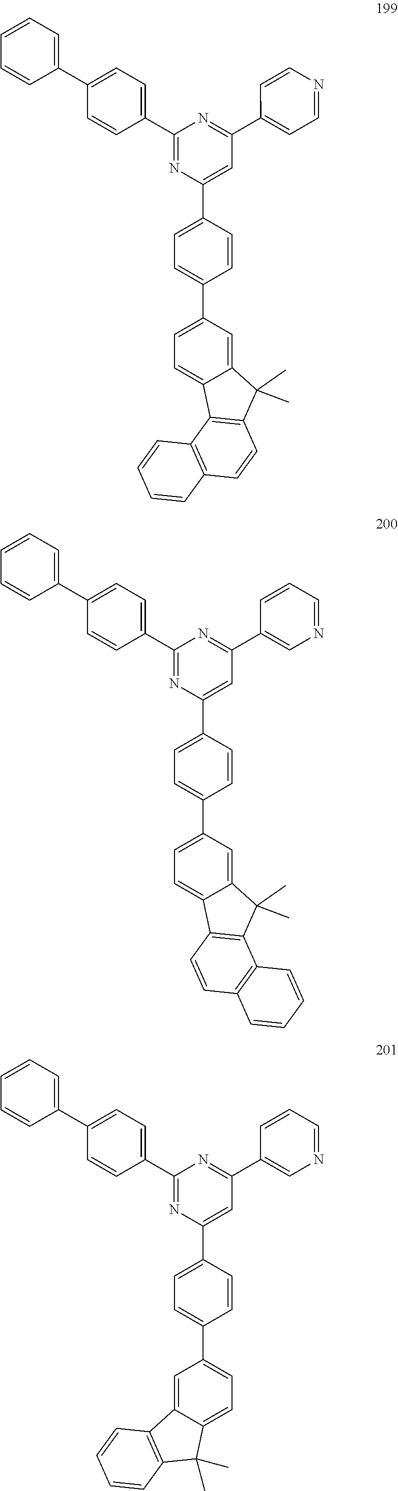 [SMILES: CC1(C)c2cc(-c3ccc(-c4cc(-c5ccncc5)nc(-c5ccc(-c6ccccc6)cc5)n4)cc3)ccc2-c2c1ccc1ccccc21 ; CC1(C)c2cc(-c3ccc(-c4cc(-c5cccnc5)nc(-c5ccc(-c6ccccc6)cc5)n4)cc3)ccc2-c2ccc3ccccc3c21 ; CC1(C)c2ccccc2-c2cc(-c3ccc(-c4cc(-c5cccnc5)nc(-c5ccc(-c6ccccc6)cc5)n4)cc3)ccc21]

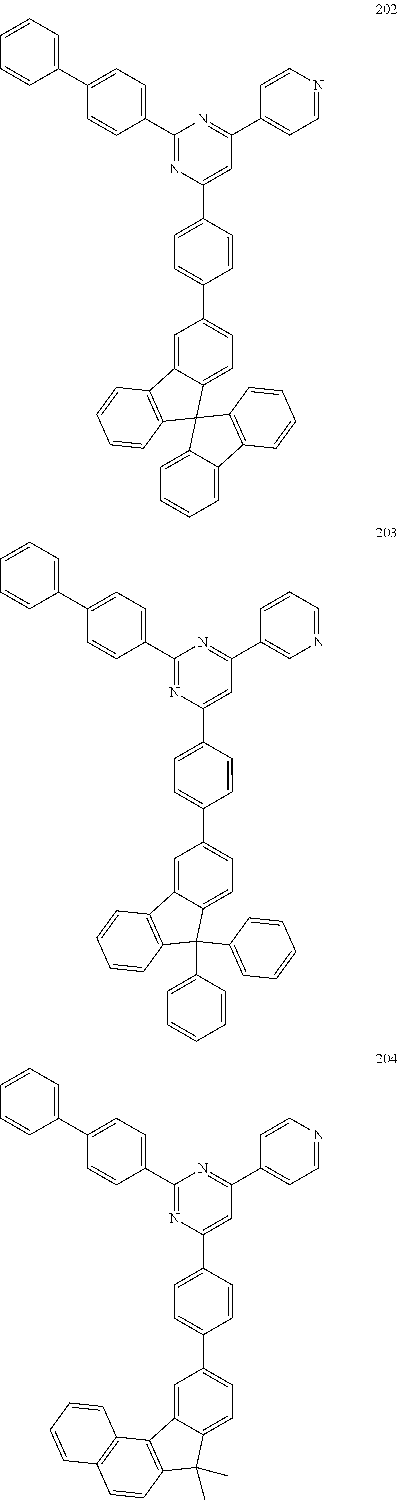 [SMILES: c1ccc(-c2ccc(-c3nc(-c4ccncc4)cc(-c4ccc(-c5ccc6c(c5)-c5ccccc5C65c6ccccc6-c6ccccc65)cc4)n3)cc2)cc1 ; c1ccc(-c2ccc(-c3nc(-c4ccc(-c5ccc6c(c5)-c5ccccc5C6(c5ccccc5)c5ccccc5)cc4)cc(-c4cccnc4)n3)cc2)cc1 ; CC1(C)c2ccc(-c3ccc(-c4cc(-c5ccncc5)nc(-c5ccc(-c6ccccc6)cc5)n4)cc3)cc2-c2c1ccc1ccccc21]

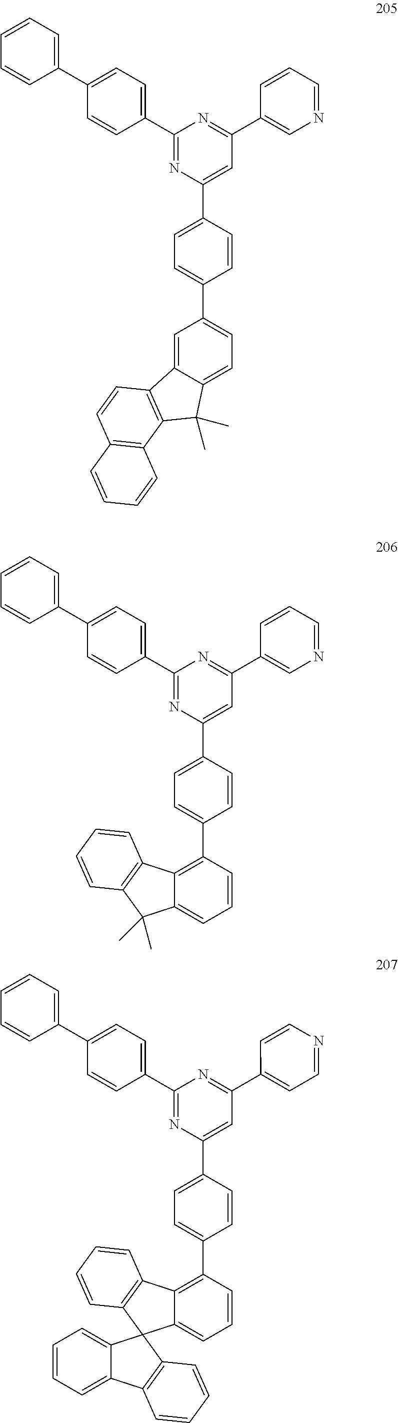 [SMILES: CC1(C)c2ccc(-c3ccc(-c4cc(-c5cccnc5)nc(-c5ccc(-c6ccccc6)cc5)n4)cc3)cc2-c2ccc3ccccc3c21 ; CC1(C)c2ccccc2-c2c(-c3ccc(-c4cc(-c5cccnc5)nc(-c5ccc(-c6ccccc6)cc5)n4)cc3)cccc21 ; c1ccc(-c2ccc(-c3nc(-c4ccncc4)cc(-c4ccc(-c5cccc6c5-c5ccccc5C65c6ccccc6-c6ccccc65)cc4)n3)cc2)cc1]

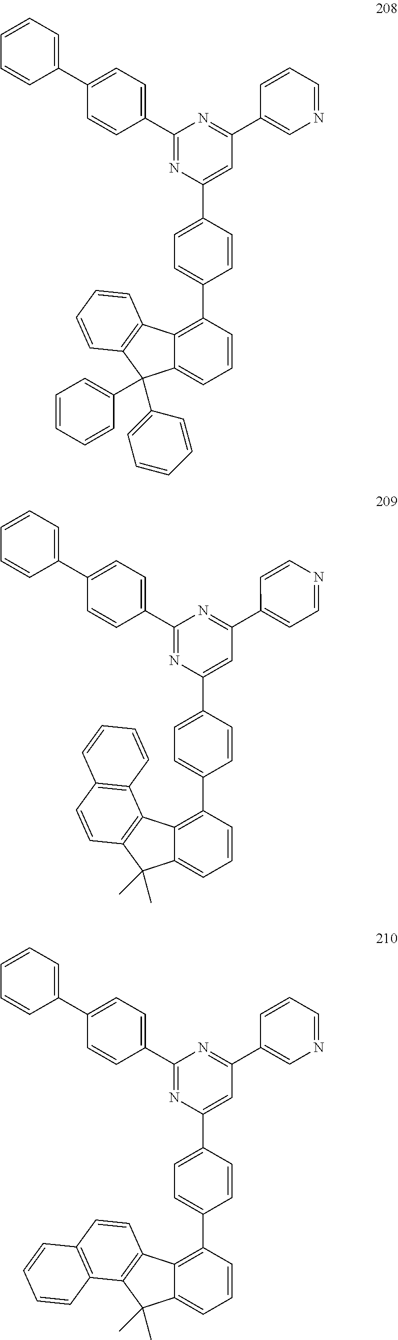 [SMILES: c1ccc(-c2ccc(-c3nc(-c4ccc(-c5cccc6c5-c5ccccc5C6(c5ccccc5)c5ccccc5)cc4)cc(-c4cccnc4)n3)cc2)cc1 ; CC1(C)c2cccc(-c3ccc(-c4cc(-c5ccncc5)nc(-c5ccc(-c6ccccc6)cc5)n4)cc3)c2-c2c1ccc1ccccc21 ; CC1(C)c2cccc(-c3ccc(-c4cc(-c5cccnc5)nc(-c5ccc(-c6ccccc6)cc5)n4)cc3)c2-c2ccc3ccccc3c21]

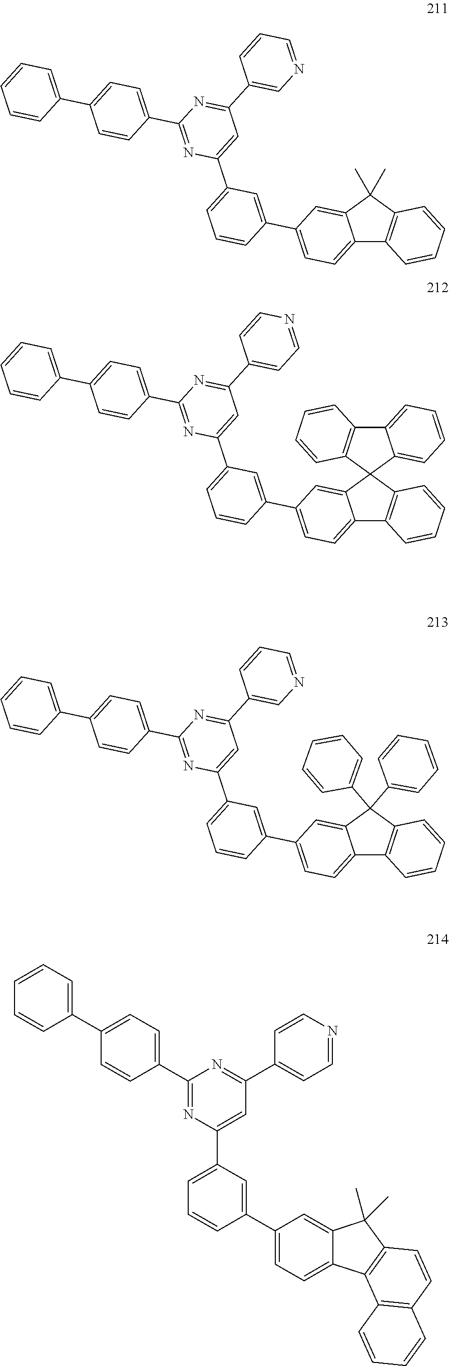 [SMILES: CC1(C)c2ccccc2-c2ccc(-c3cccc(-c4cc(-c5cccnc5)nc(-c5ccc(-c6ccccc6)cc5)n4)c3)cc21 ; c1ccc(-c2ccc(-c3nc(-c4ccncc4)cc(-c4cccc(-c5ccc6c(c5)C5(c7ccccc7-c7ccccc75)c5ccccc5-6)c4)n3)cc2)cc1 ; c1ccc(-c2ccc(-c3nc(-c4cccnc4)cc(-c4cccc(-c5ccc6c(c5)C(c5ccccc5)(c5ccccc5)c5ccccc5-6)c4)n3)cc2)cc1 ; CC1(C)c2cc(-c3cccc(-c4cc(-c5ccncc5)nc(-c5ccc(-c6ccccc6)cc5)n4)c3)ccc2-c2c1ccc1ccccc21]

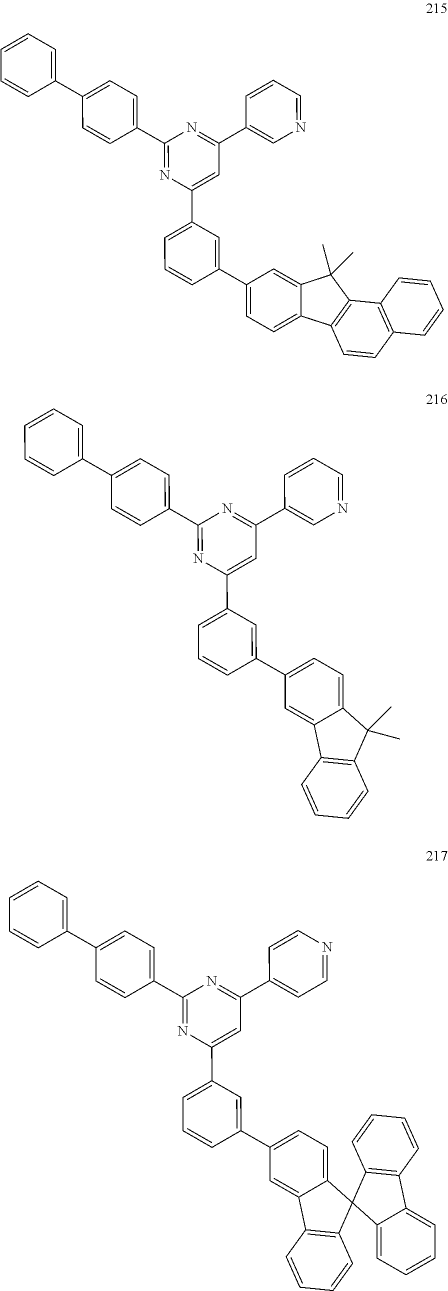 [SMILES: CC1(C)c2cc(-c3cccc(-c4cc(-c5cccnc5)nc(-c5ccc(-c6ccccc6)cc5)n4)c3)ccc2-c2ccc3ccccc3c21 ; CC1(C)c2ccccc2-c2cc(-c3cccc(-c4cc(-c5cccnc5)nc(-c5ccc(-c6ccccc6)cc5)n4)c3)ccc21 ; c1ccc(-c2ccc(-c3nc(-c4ccncc4)cc(-c4cccc(-c5ccc6c(c5)-c5ccccc5C65c6ccccc6-c6ccccc65)c4)n3)cc2)cc1]

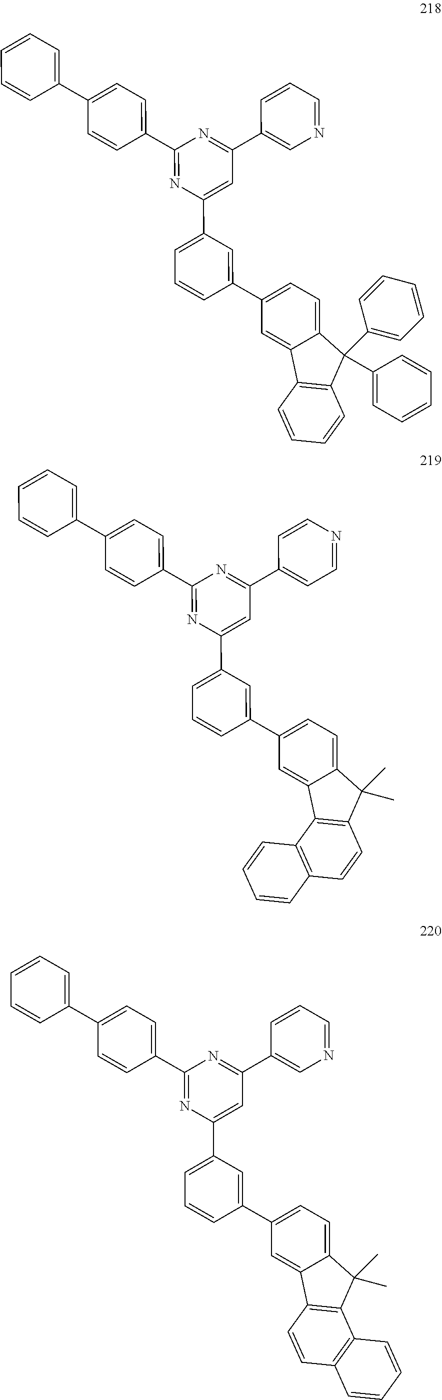 [SMILES: c1ccc(-c2ccc(-c3nc(-c4cccnc4)cc(-c4cccc(-c5ccc6c(c5)-c5ccccc5C6(c5ccccc5)c5ccccc5)c4)n3)cc2)cc1 ; CC1(C)c2ccc(-c3cccc(-c4cc(-c5ccncc5)nc(-c5ccc(-c6ccccc6)cc5)n4)c3)cc2-c2c1ccc1ccccc21 ; CC1(C)c2ccc(-c3cccc(-c4cc(-c5cccnc5)nc(-c5ccc(-c6ccccc6)cc5)n4)c3)cc2-c2ccc3ccccc3c21]

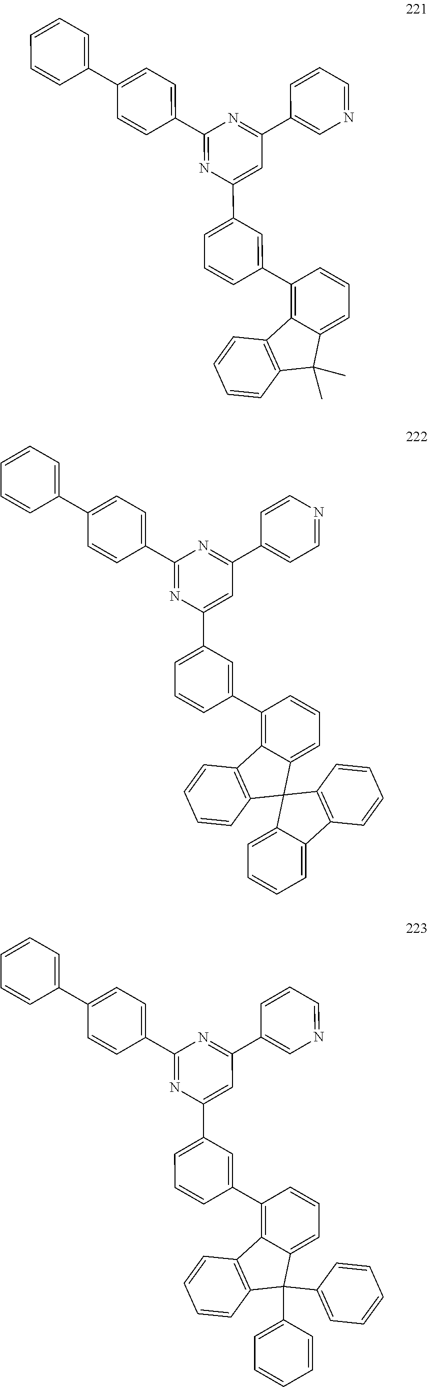 [SMILES: CC1(C)c2ccccc2-c2c(-c3cccc(-c4cc(-c5cccnc5)nc(-c5ccc(-c6ccccc6)cc5)n4)c3)cccc21 ; c1ccc(-c2ccc(-c3nc(-c4ccncc4)cc(-c4cccc(-c5cccc6c5-c5ccccc5C65c6ccccc6-c6ccccc65)c4)n3)cc2)cc1 ; c1ccc(-c2ccc(-c3nc(-c4cccnc4)cc(-c4cccc(-c5cccc6c5-c5ccccc5C6(c5ccccc5)c5ccccc5)c4)n3)cc2)cc1]

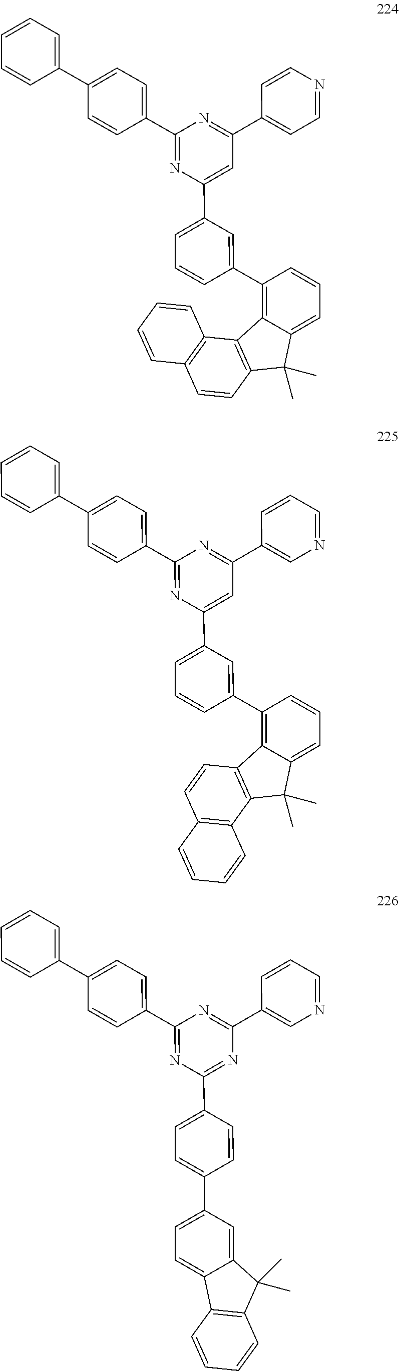 [SMILES: CC1(C)c2cccc(-c3cccc(-c4cc(-c5ccncc5)nc(-c5ccc(-c6ccccc6)cc5)n4)c3)c2-c2c1ccc1ccccc21 ; CC1(C)c2cccc(-c3cccc(-c4cc(-c5cccnc5)nc(-c5ccc(-c6ccccc6)cc5)n4)c3)c2-c2ccc3ccccc3c21 ; CC1(C)c2ccccc2-c2ccc(-c3ccc(-c4nc(-c5ccc(-c6ccccc6)cc5)nc(-c5cccnc5)n4)cc3)cc21]

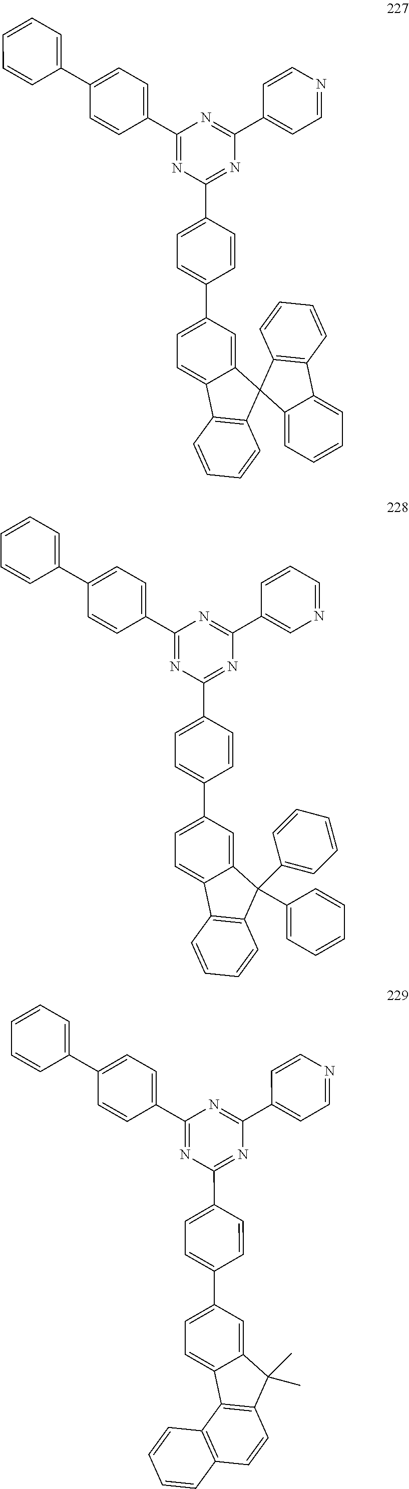 [SMILES: c1ccc(-c2ccc(-c3nc(-c4ccncc4)nc(-c4ccc(-c5ccc6c(c5)C5(c7ccccc7-c7ccccc75)c5ccccc5-6)cc4)n3)cc2)cc1 ; c1ccc(-c2ccc(-c3nc(-c4ccc(-c5ccc6c(c5)C(c5ccccc5)(c5ccccc5)c5ccccc5-6)cc4)nc(-c4cccnc4)n3)cc2)cc1 ; CC1(C)c2cc(-c3ccc(-c4nc(-c5ccncc5)nc(-c5ccc(-c6ccccc6)cc5)n4)cc3)ccc2-c2c1ccc1ccccc21]

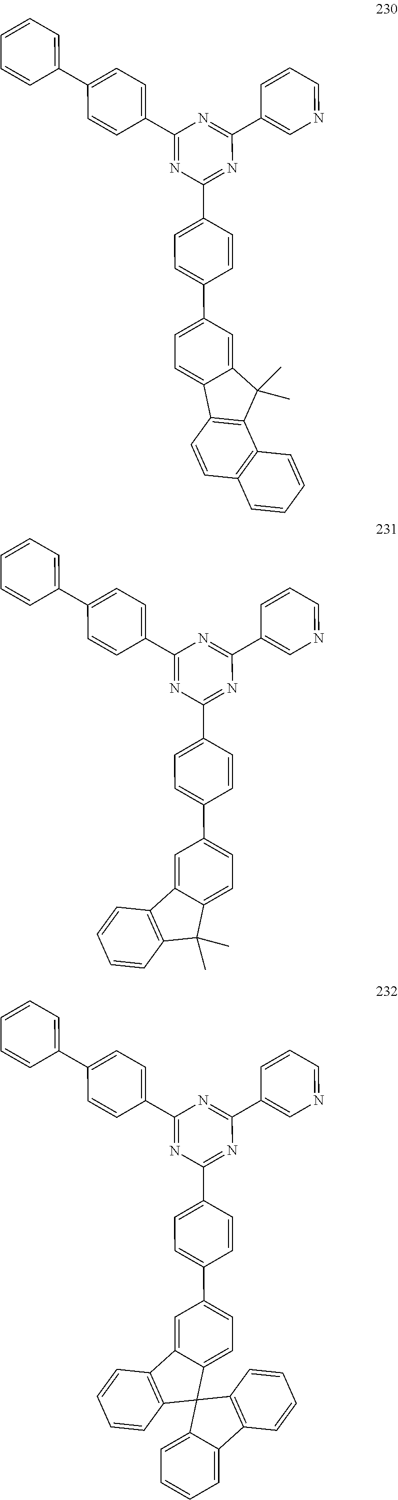 [SMILES: CC1(C)c2cc(-c3ccc(-c4nc(-c5ccc(-c6ccccc6)cc5)nc(-c5cccnc5)n4)cc3)ccc2-c2ccc3ccccc3c21 ; CC1(C)c2ccccc2-c2cc(-c3ccc(-c4nc(-c5ccc(-c6ccccc6)cc5)nc(-c5cccnc5)n4)cc3)ccc21 ; c1ccc(-c2ccc(-c3nc(-c4ccc(-c5ccc6c(c5)-c5ccccc5C65c6ccccc6-c6ccccc65)cc4)nc(-c4cccnc4)n3)cc2)cc1]

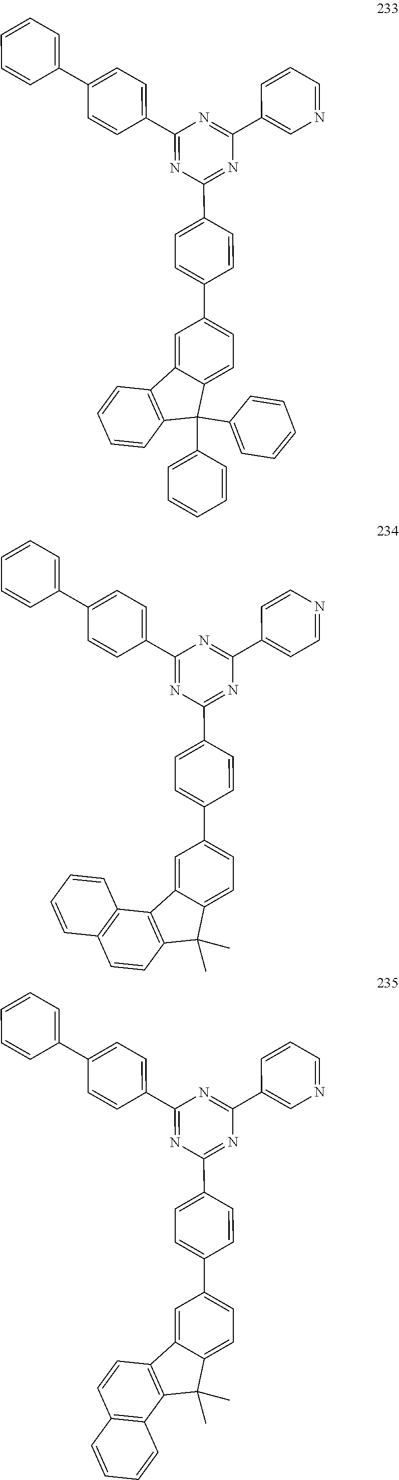 [SMILES: c1ccc(-c2ccc(-c3nc(-c4ccc(-c5ccc6c(c5)-c5ccccc5C6(c5ccccc5)c5ccccc5)cc4)nc(-c4cccnc4)n3)cc2)cc1 ; CC1(C)c2ccc(-c3ccc(-c4nc(-c5ccncc5)nc(-c5ccc(-c6ccccc6)cc5)n4)cc3)cc2-c2c1ccc1ccccc21 ; CC1(C)c2ccc(-c3ccc(-c4nc(-c5ccc(-c6ccccc6)cc5)nc(-c5cccnc5)n4)cc3)cc2-c2ccc3ccccc3c21]

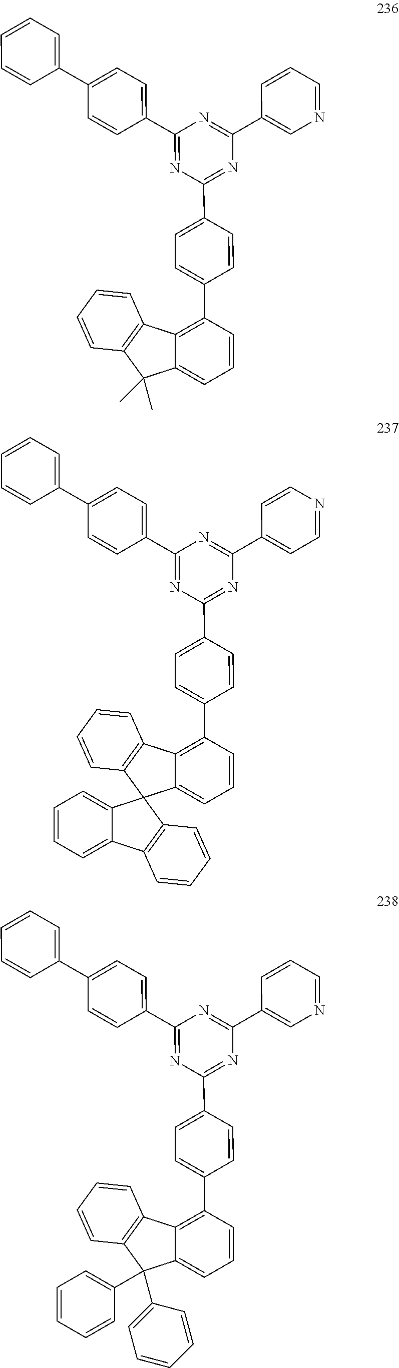 [SMILES: CC1(C)c2ccccc2-c2c(-c3ccc(-c4nc(-c5ccc(-c6ccccc6)cc5)nc(-c5cccnc5)n4)cc3)cccc21 ; c1ccc(-c2ccc(-c3nc(-c4ccncc4)nc(-c4ccc(-c5cccc6c5-c5ccccc5C65c6ccccc6-c6ccccc65)cc4)n3)cc2)cc1 ; c1ccc(-c2ccc(-c3nc(-c4ccc(-c5cccc6c5-c5ccccc5C6(c5ccccc5)c5ccccc5)cc4)nc(-c4cccnc4)n3)cc2)cc1]

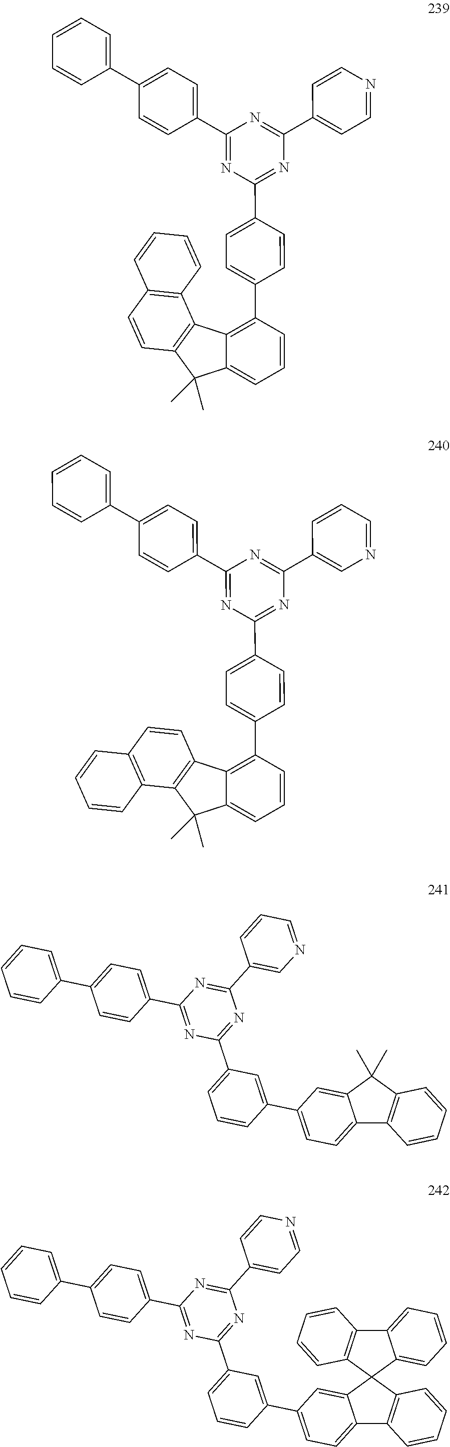 [SMILES: CC1(C)c2cccc(-c3ccc(-c4nc(-c5ccncc5)nc(-c5ccc(-c6ccccc6)cc5)n4)cc3)c2-c2c1ccc1ccccc21 ; CC1(C)c2cccc(-c3ccc(-c4nc(-c5ccc(-c6ccccc6)cc5)nc(-c5cccnc5)n4)cc3)c2-c2ccc3ccccc3c21 ; CC1(C)c2ccccc2-c2ccc(-c3cccc(-c4nc(-c5ccc(-c6ccccc6)cc5)nc(-c5cccnc5)n4)c3)cc21 ; c1ccc(-c2ccc(-c3nc(-c4ccncc4)nc(-c4cccc(-c5ccc6c(c5)C5(c7ccccc7-c7ccccc75)c5ccccc5-6)c4)n3)cc2)cc1]

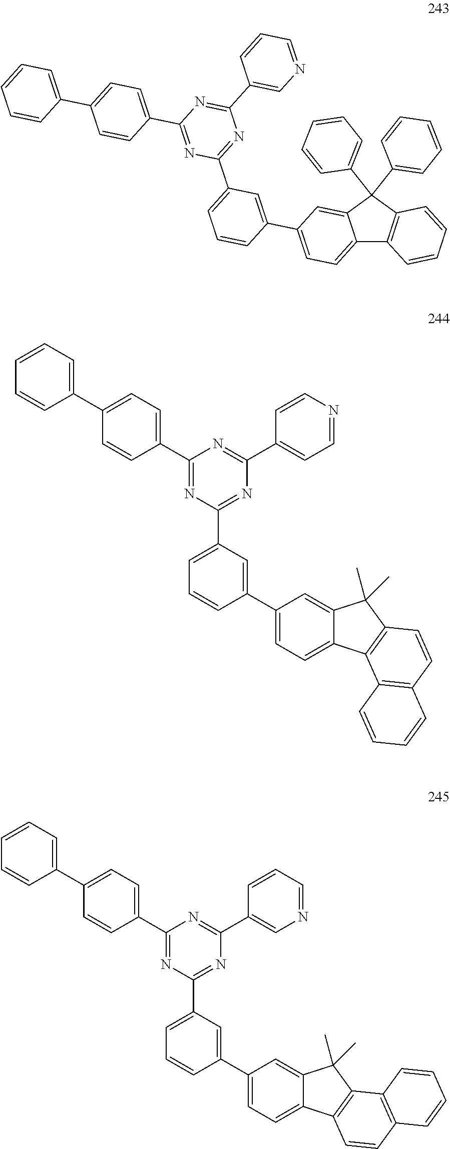 [SMILES: c1ccc(-c2ccc(-c3nc(-c4cccnc4)nc(-c4cccc(-c5ccc6c(c5)C(c5ccccc5)(c5ccccc5)c5ccccc5-6)c4)n3)cc2)cc1 ; CC1(C)c2cc(-c3cccc(-c4nc(-c5ccncc5)nc(-c5ccc(-c6ccccc6)cc5)n4)c3)ccc2-c2c1ccc1ccccc21 ; CC1(C)c2cc(-c3cccc(-c4nc(-c5ccc(-c6ccccc6)cc5)nc(-c5cccnc5)n4)c3)ccc2-c2ccc3ccccc3c21]

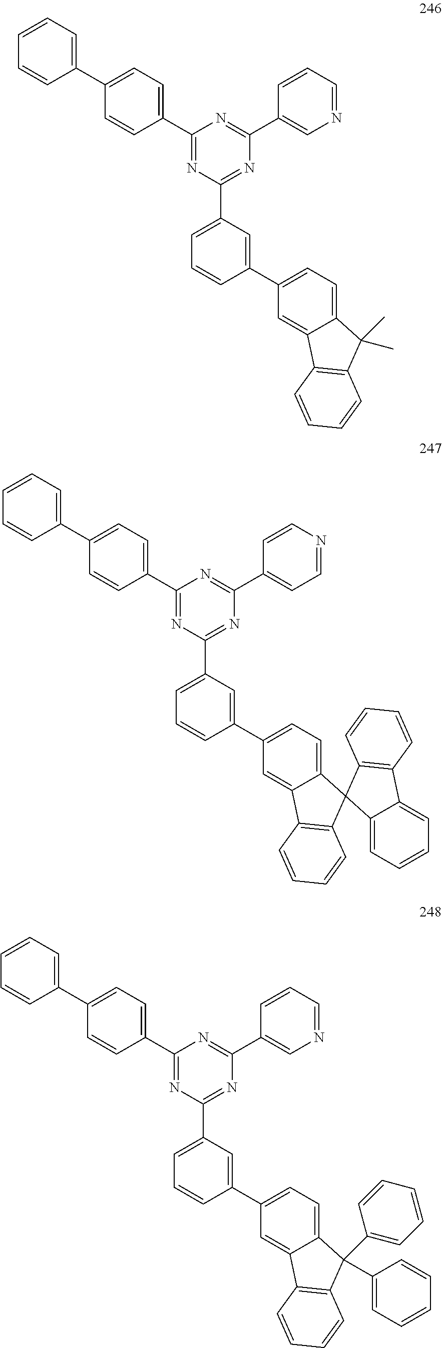 [SMILES: CC1(C)c2ccccc2-c2cc(-c3cccc(-c4nc(-c5ccc(-c6ccccc6)cc5)nc(-c5cccnc5)n4)c3)ccc21 ; c1ccc(-c2ccc(-c3nc(-c4ccncc4)nc(-c4cccc(-c5ccc6c(c5)-c5ccccc5C65c6ccccc6-c6ccccc65)c4)n3)cc2)cc1 ; c1ccc(-c2ccc(-c3nc(-c4cccnc4)nc(-c4cccc(-c5ccc6c(c5)-c5ccccc5C6(c5ccccc5)c5ccccc5)c4)n3)cc2)cc1]

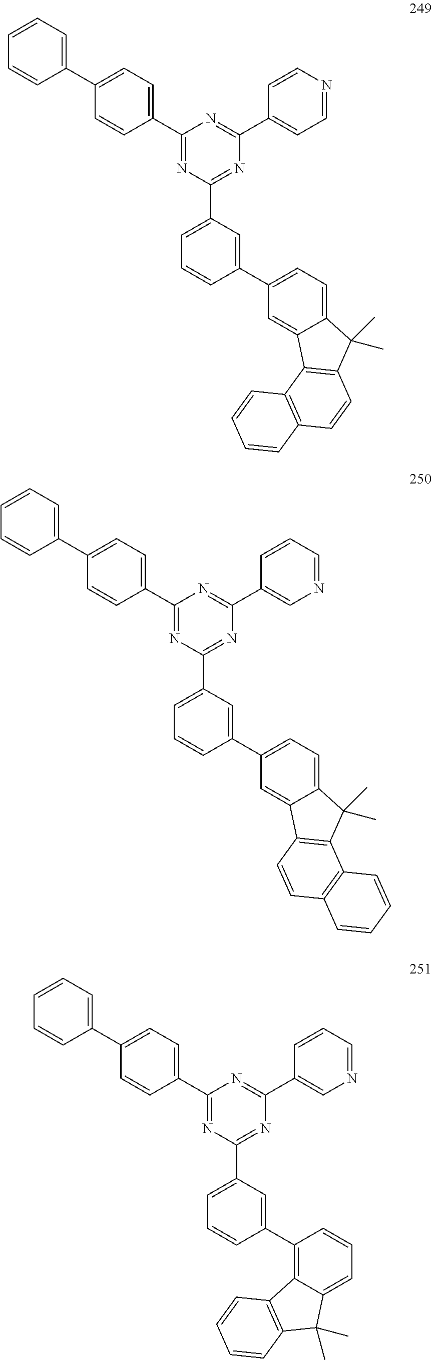 [SMILES: CC1(C)c2ccc(-c3cccc(-c4nc(-c5ccncc5)nc(-c5ccc(-c6ccccc6)cc5)n4)c3)cc2-c2c1ccc1ccccc21 ; CC1(C)c2ccc(-c3cccc(-c4nc(-c5ccc(-c6ccccc6)cc5)nc(-c5cccnc5)n4)c3)cc2-c2ccc3ccccc3c21 ; CC1(C)c2ccccc2-c2c(-c3cccc(-c4nc(-c5ccc(-c6ccccc6)cc5)nc(-c5cccnc5)n4)c3)cccc21]

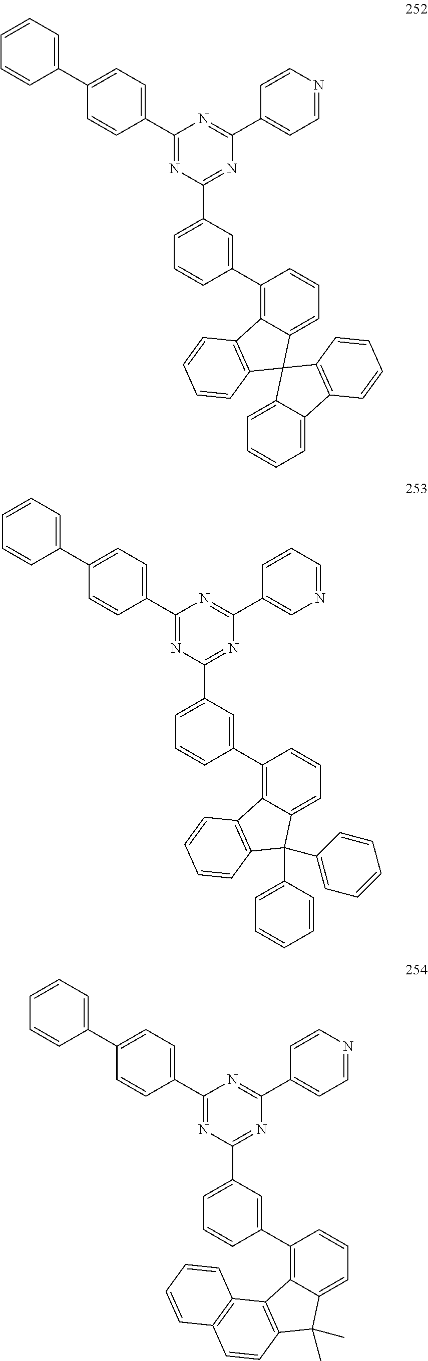 [SMILES: c1ccc(-c2ccc(-c3nc(-c4ccncc4)nc(-c4cccc(-c5cccc6c5-c5ccccc5C65c6ccccc6-c6ccccc65)c4)n3)cc2)cc1 ; c1ccc(-c2ccc(-c3nc(-c4cccnc4)nc(-c4cccc(-c5cccc6c5-c5ccccc5C6(c5ccccc5)c5ccccc5)c4)n3)cc2)cc1 ; CC1(C)c2cccc(-c3cccc(-c4nc(-c5ccncc5)nc(-c5ccc(-c6ccccc6)cc5)n4)c3)c2-c2c1ccc1ccccc21]

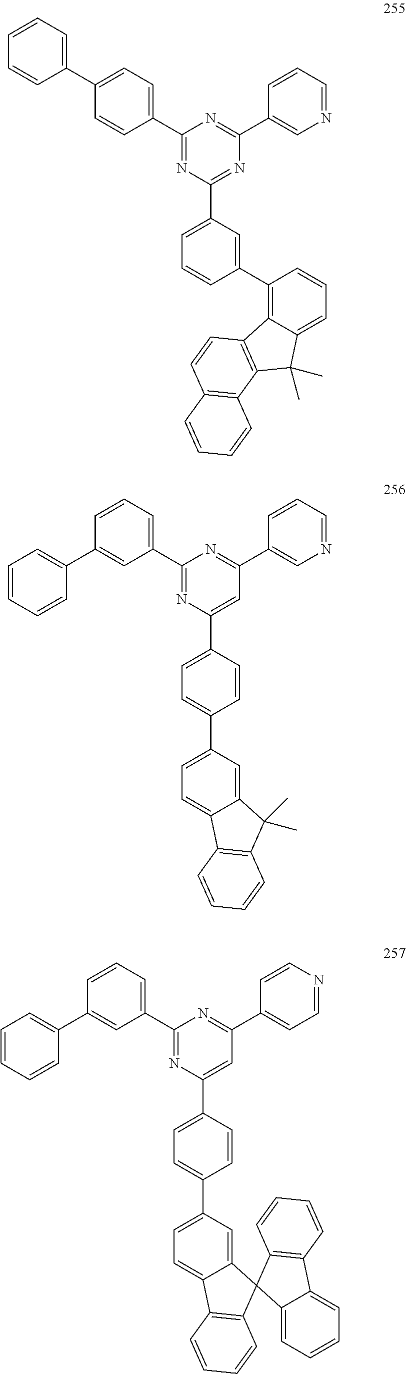 [SMILES: CC1(C)c2cccc(-c3cccc(-c4nc(-c5ccc(-c6ccccc6)cc5)nc(-c5cccnc5)n4)c3)c2-c2ccc3ccccc3c21 ; CC1(C)c2ccccc2-c2ccc(-c3ccc(-c4cc(-c5cccnc5)nc(-c5cccc(-c6ccccc6)c5)n4)cc3)cc21 ; c1ccc(-c2cccc(-c3nc(-c4ccncc4)cc(-c4ccc(-c5ccc6c(c5)C5(c7ccccc7-c7ccccc75)c5ccccc5-6)cc4)n3)c2)cc1]

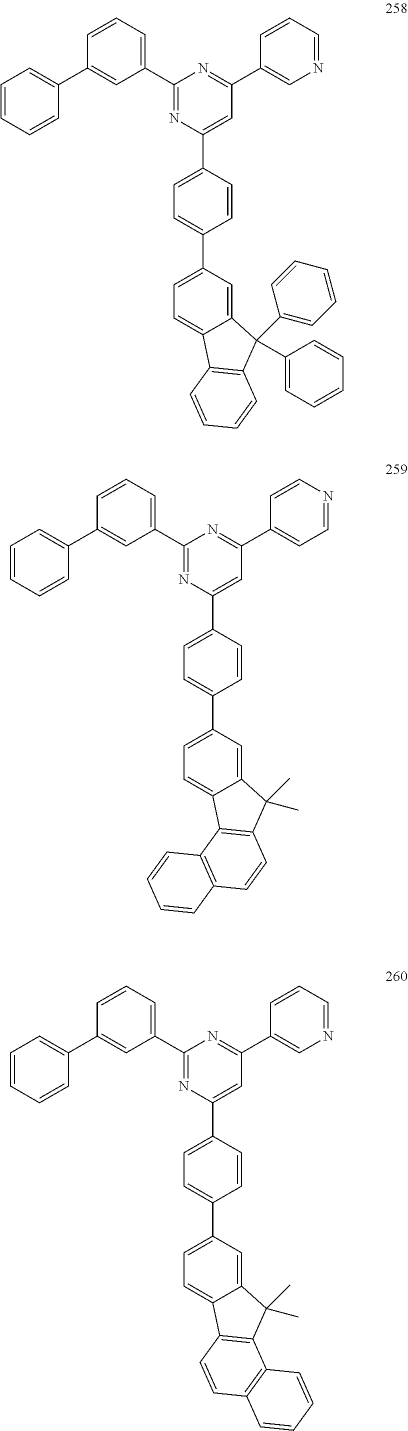 [SMILES: c1ccc(-c2cccc(-c3nc(-c4ccc(-c5ccc6c(c5)C(c5ccccc5)(c5ccccc5)c5ccccc5-6)cc4)cc(-c4cccnc4)n3)c2)cc1 ; CC1(C)c2cc(-c3ccc(-c4cc(-c5ccncc5)nc(-c5cccc(-c6ccccc6)c5)n4)cc3)ccc2-c2c1ccc1ccccc21 ; CC1(C)c2cc(-c3ccc(-c4cc(-c5cccnc5)nc(-c5cccc(-c6ccccc6)c5)n4)cc3)ccc2-c2ccc3ccccc3c21]

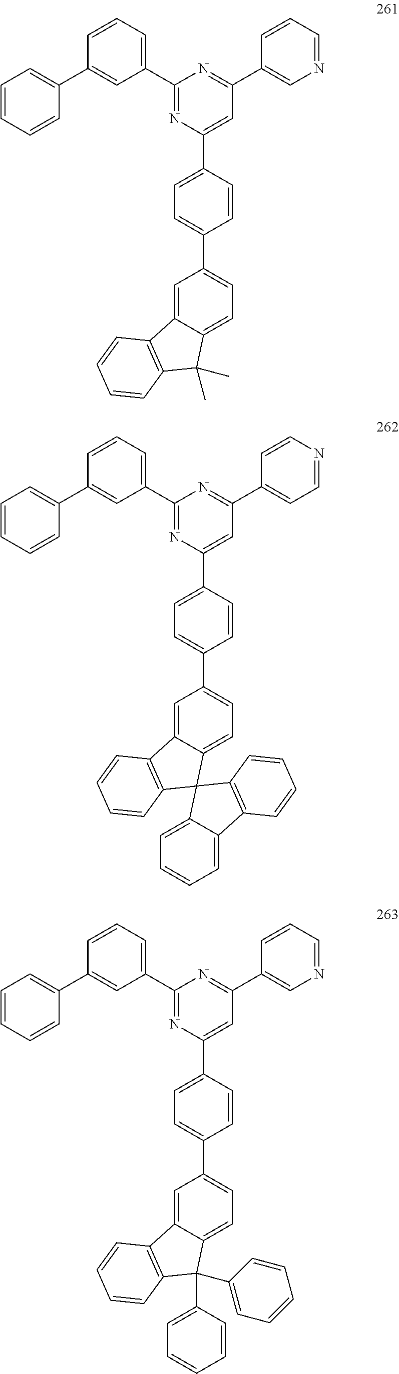

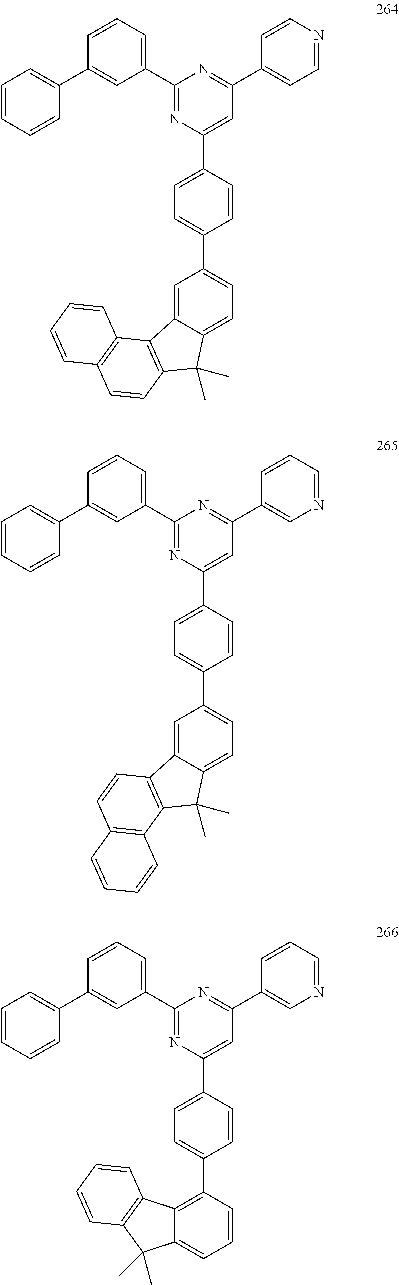 [SMILES: CC1(C)c2ccc(-c3ccc(-c4cc(-c5ccncc5)nc(-c5cccc(-c6ccccc6)c5)n4)cc3)cc2-c2c1ccc1ccccc21 ; CC1(C)c2ccc(-c3ccc(-c4cc(-c5cccnc5)nc(-c5cccc(-c6ccccc6)c5)n4)cc3)cc2-c2ccc3ccccc3c21 ; CC1(C)c2ccccc2-c2c(-c3ccc(-c4cc(-c5cccnc5)nc(-c5cccc(-c6ccccc6)c5)n4)cc3)cccc21]

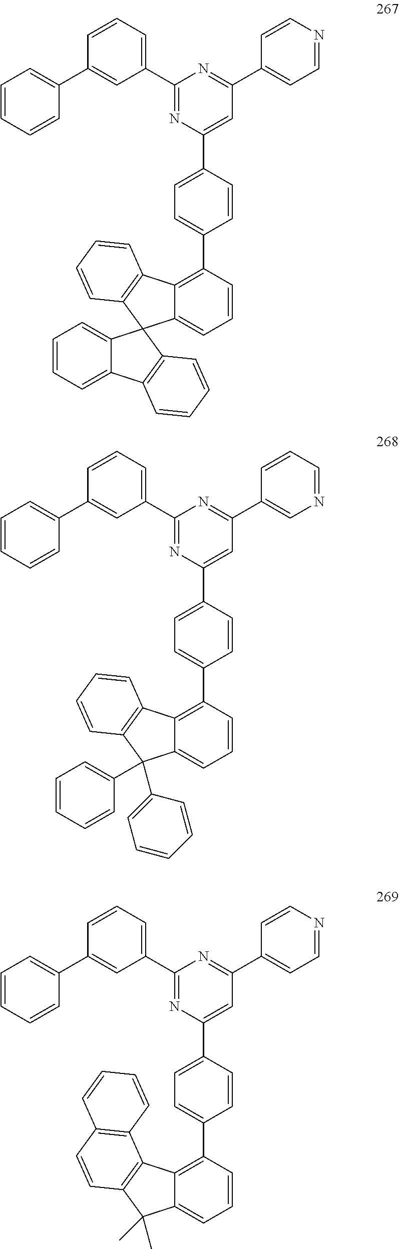 [SMILES: c1ccc(-c2cccc(-c3nc(-c4ccncc4)cc(-c4ccc(-c5cccc6c5-c5ccccc5C65c6ccccc6-c6ccccc65)cc4)n3)c2)cc1 ; c1ccc(-c2cccc(-c3nc(-c4ccc(-c5cccc6c5-c5ccccc5C6(c5ccccc5)c5ccccc5)cc4)cc(-c4cccnc4)n3)c2)cc1 ; CC1(C)c2cccc(-c3ccc(-c4cc(-c5ccncc5)nc(-c5cccc(-c6ccccc6)c5)n4)cc3)c2-c2c1ccc1ccccc21]

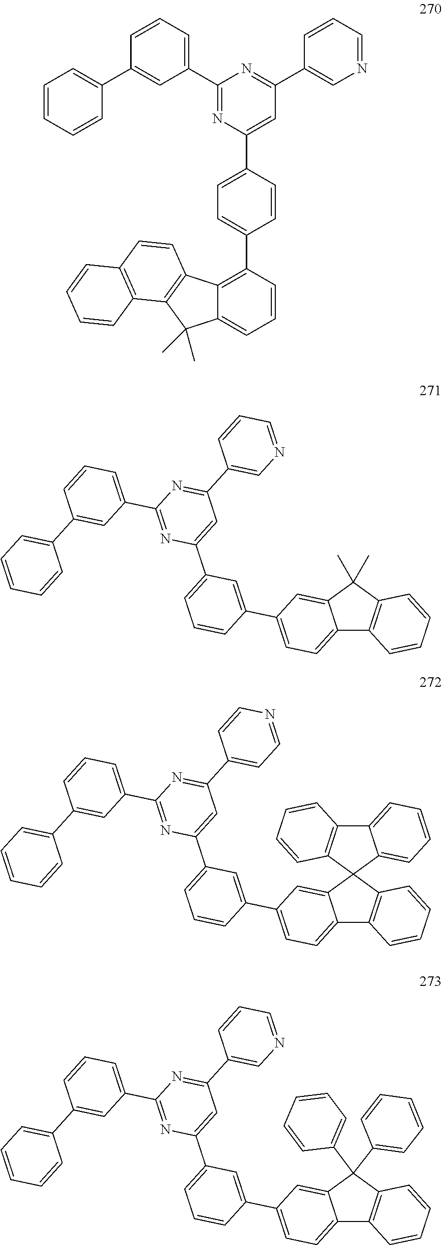 [SMILES: CC1(C)c2cccc(-c3ccc(-c4cc(-c5cccnc5)nc(-c5cccc(-c6ccccc6)c5)n4)cc3)c2-c2ccc3ccccc3c21 ; CC1(C)c2ccccc2-c2ccc(-c3cccc(-c4cc(-c5cccnc5)nc(-c5cccc(-c6ccccc6)c5)n4)c3)cc21 ; c1ccc(-c2cccc(-c3nc(-c4ccncc4)cc(-c4cccc(-c5ccc6c(c5)C5(c7ccccc7-c7ccccc75)c5ccccc5-6)c4)n3)c2)cc1 ; c1ccc(-c2cccc(-c3nc(-c4cccnc4)cc(-c4cccc(-c5ccc6c(c5)C(c5ccccc5)(c5ccccc5)c5ccccc5-6)c4)n3)c2)cc1]

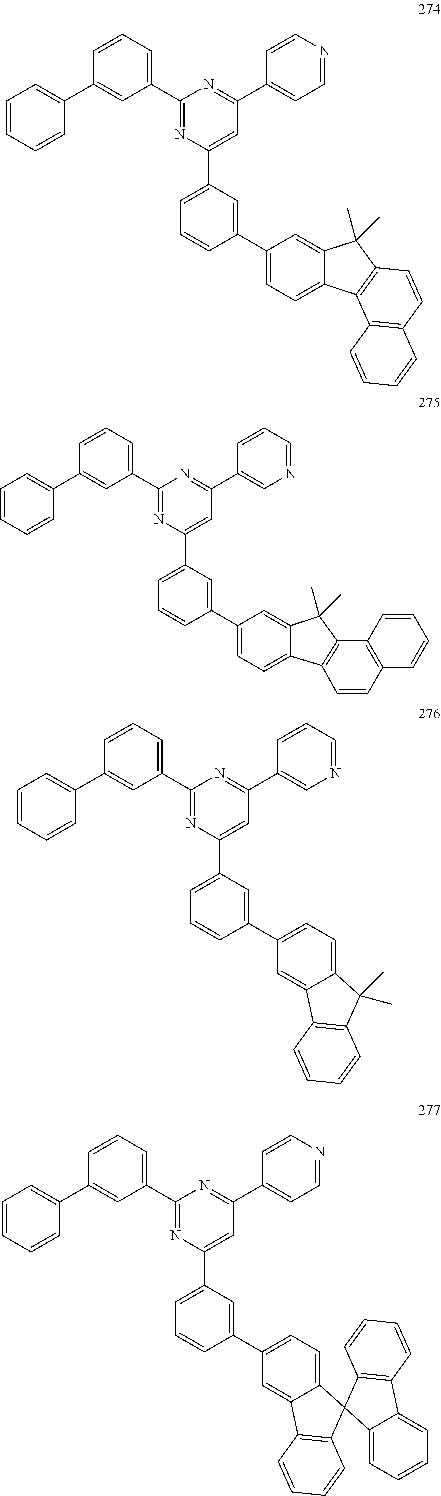

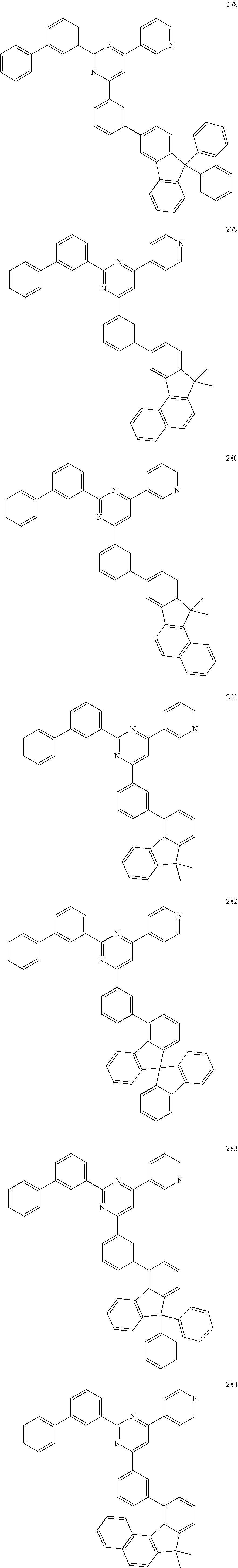

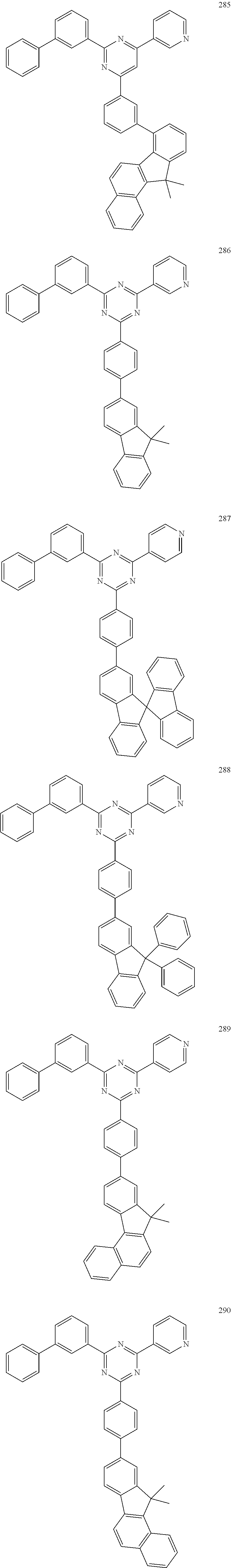

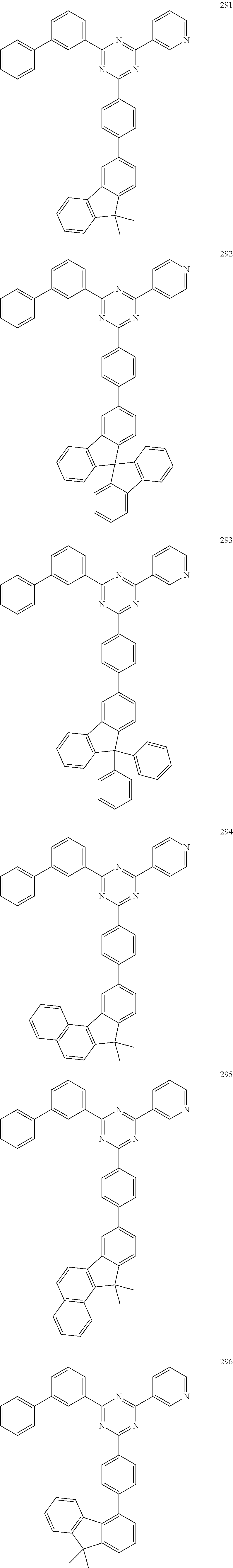

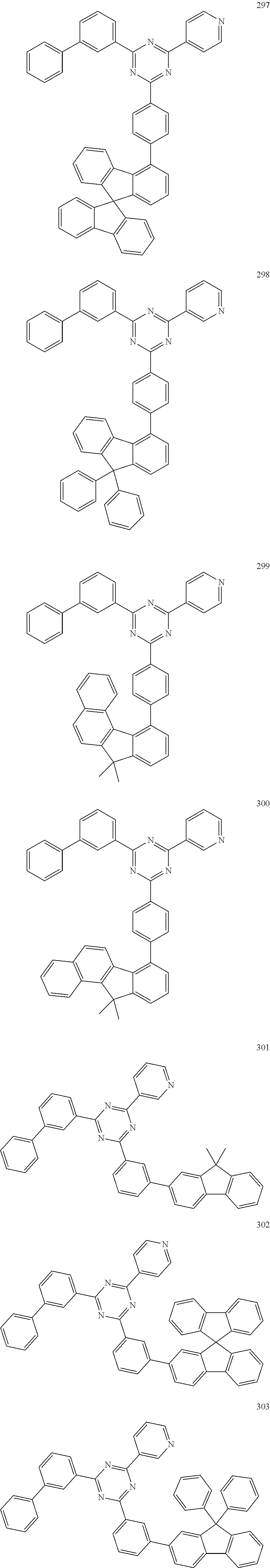

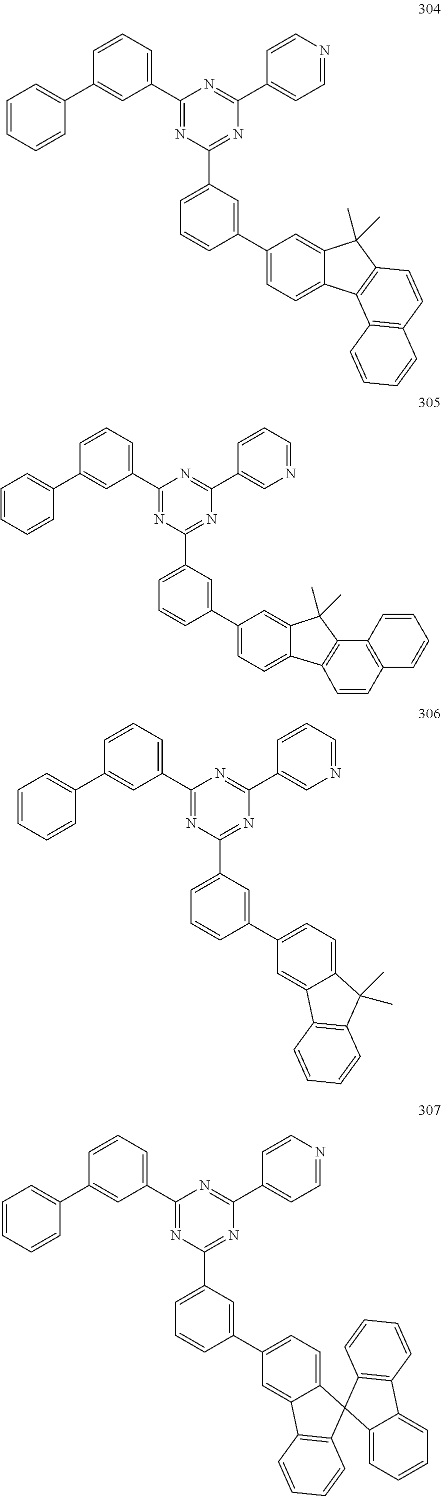

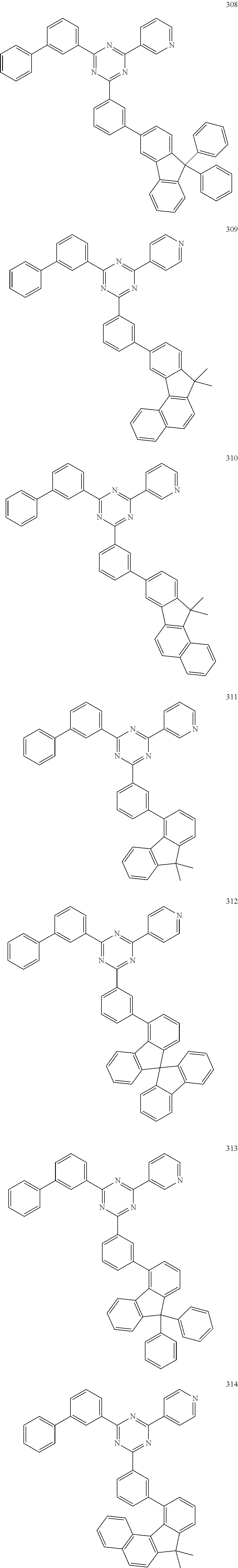

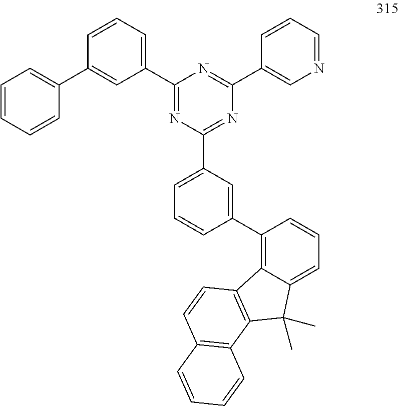 [SMILES: CC1(C)c2cccc(-c3cccc(-c4nc(-c5cccnc5)nc(-c5cccc(-c6ccccc6)c5)n4)c3)c2-c2ccc3ccccc3c21]

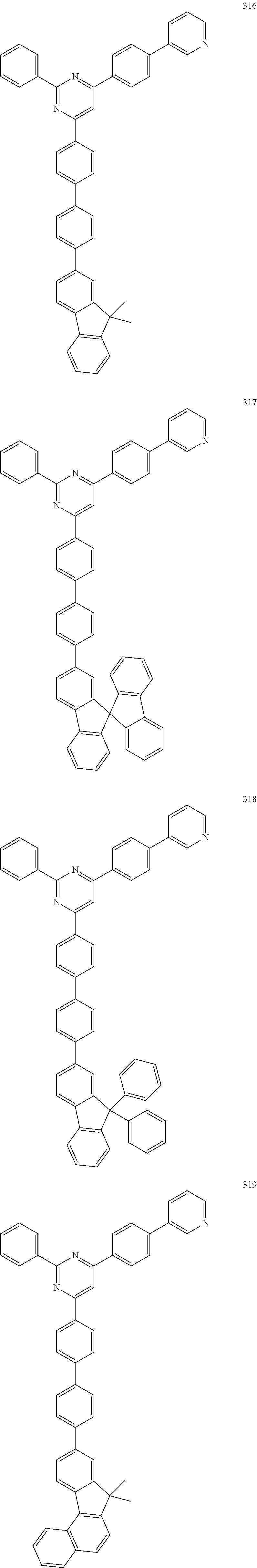 [SMILES: CC1(C)c2ccccc2-c2ccc(-c3ccc(-c4ccc(-c5cc(-c6ccc(-c7cccnc7)cc6)nc(-c6ccccc6)n5)cc4)cc3)cc21 ; c1ccc(-c2nc(-c3ccc(-c4ccc(-c5ccc6c(c5)C5(c7ccccc7-c7ccccc75)c5ccccc5-6)cc4)cc3)cc(-c3ccc(-c4cccnc4)cc3)n2)cc1 ; c1ccc(-c2nc(-c3ccc(-c4ccc(-c5ccc6c(c5)C(c5ccccc5)(c5ccccc5)c5ccccc5-6)cc4)cc3)cc(-c3ccc(-c4cccnc4)cc3)n2)cc1 ; CC1(C)c2cc(-c3ccc(-c4ccc(-c5cc(-c6ccc(-c7cccnc7)cc6)nc(-c6ccccc6)n5)cc4)cc3)ccc2-c2c1ccc1ccccc21]

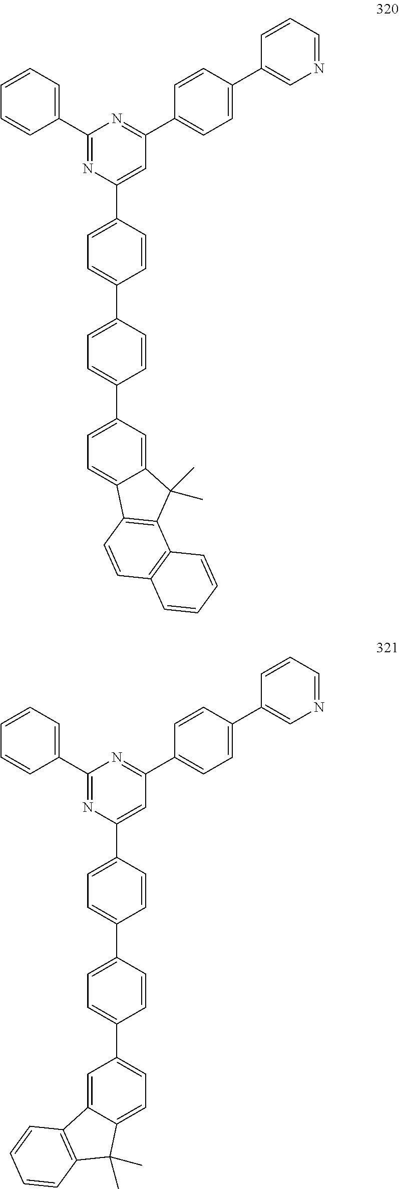 [SMILES: CC1(C)c2cc(-c3ccc(-c4ccc(-c5cc(-c6ccc(-c7cccnc7)cc6)nc(-c6ccccc6)n5)cc4)cc3)ccc2-c2ccc3ccccc3c21 ; CC1(C)c2ccccc2-c2cc(-c3ccc(-c4ccc(-c5cc(-c6ccc(-c7cccnc7)cc6)nc(-c6ccccc6)n5)cc4)cc3)ccc21]

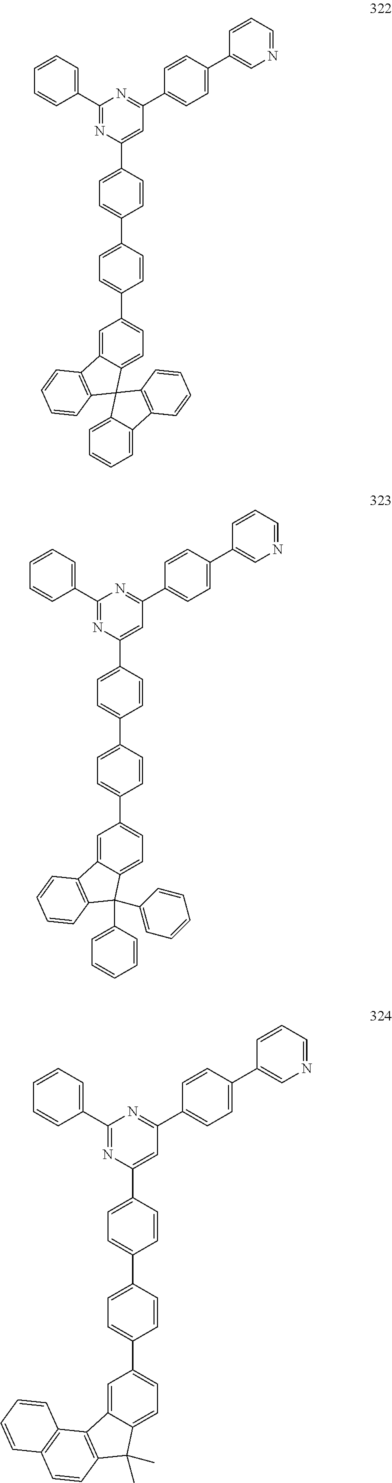 [SMILES: c1ccc(-c2nc(-c3ccc(-c4ccc(-c5ccc6c(c5)-c5ccccc5C65c6ccccc6-c6ccccc65)cc4)cc3)cc(-c3ccc(-c4cccnc4)cc3)n2)cc1 ; c1ccc(-c2nc(-c3ccc(-c4ccc(-c5ccc6c(c5)-c5ccccc5C6(c5ccccc5)c5ccccc5)cc4)cc3)cc(-c3ccc(-c4cccnc4)cc3)n2)cc1 ; CC1(C)c2ccc(-c3ccc(-c4ccc(-c5cc(-c6ccc(-c7cccnc7)cc6)nc(-c6ccccc6)n5)cc4)cc3)cc2-c2c1ccc1ccccc21]

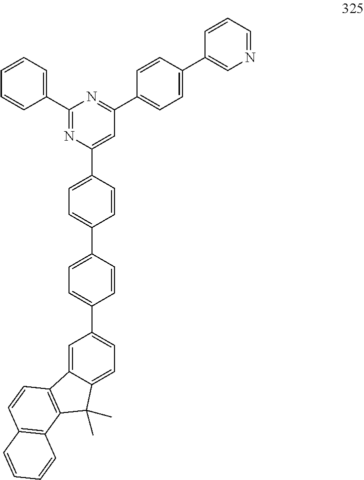 [SMILES: CC1(C)c2ccc(-c3ccc(-c4ccc(-c5cc(-c6ccc(-c7cccnc7)cc6)nc(-c6ccccc6)n5)cc4)cc3)cc2-c2ccc3ccccc3c21]

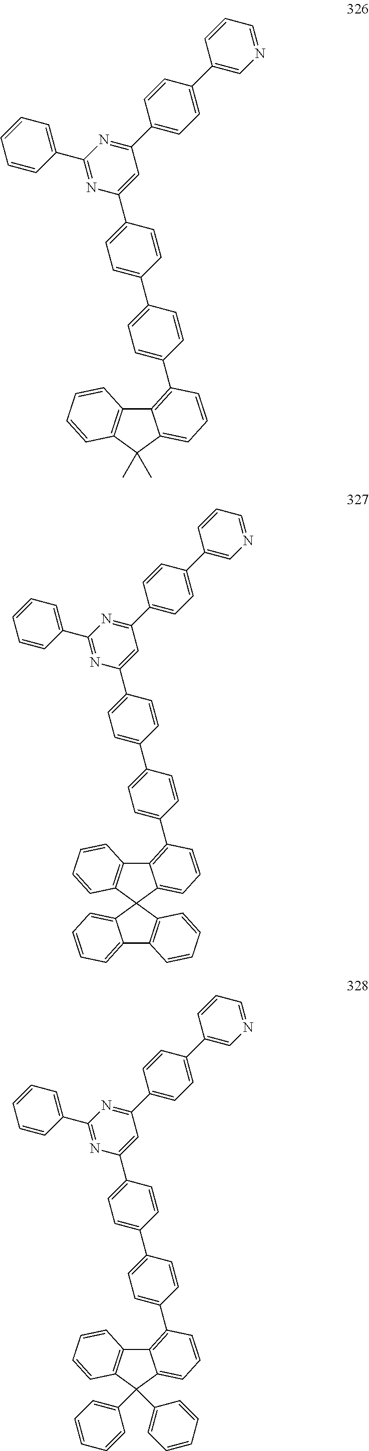 [SMILES: CC1(C)c2ccccc2-c2c(-c3ccc(-c4ccc(-c5cc(-c6ccc(-c7cccnc7)cc6)nc(-c6ccccc6)n5)cc4)cc3)cccc21 ; c1ccc(-c2nc(-c3ccc(-c4ccc(-c5cccc6c5-c5ccccc5C65c6ccccc6-c6ccccc65)cc4)cc3)cc(-c3ccc(-c4cccnc4)cc3)n2)cc1 ; c1ccc(-c2nc(-c3ccc(-c4ccc(-c5cccc6c5-c5ccccc5C6(c5ccccc5)c5ccccc5)cc4)cc3)cc(-c3ccc(-c4cccnc4)cc3)n2)cc1]

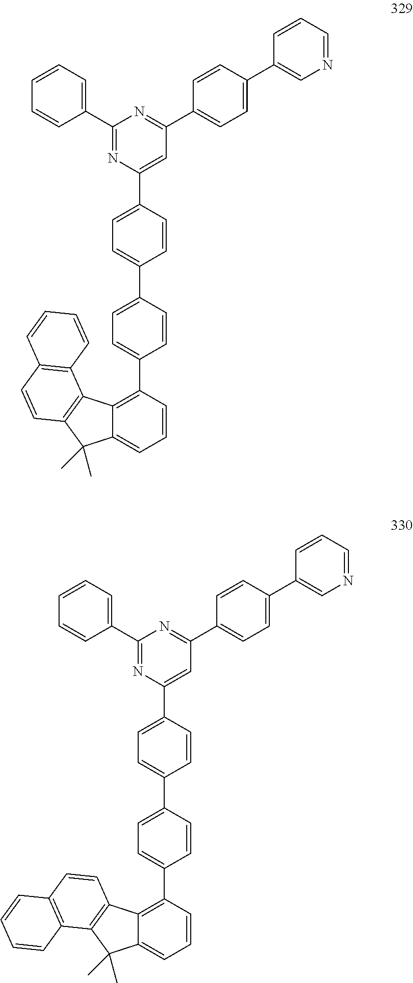 [SMILES: CC1(C)c2cccc(-c3ccc(-c4ccc(-c5cc(-c6ccc(-c7cccnc7)cc6)nc(-c6ccccc6)n5)cc4)cc3)c2-c2c1ccc1ccccc21 ; CC1(C)c2cccc(-c3ccc(-c4ccc(-c5cc(-c6ccc(-c7cccnc7)cc6)nc(-c6ccccc6)n5)cc4)cc3)c2-c2ccc3ccccc3c21]

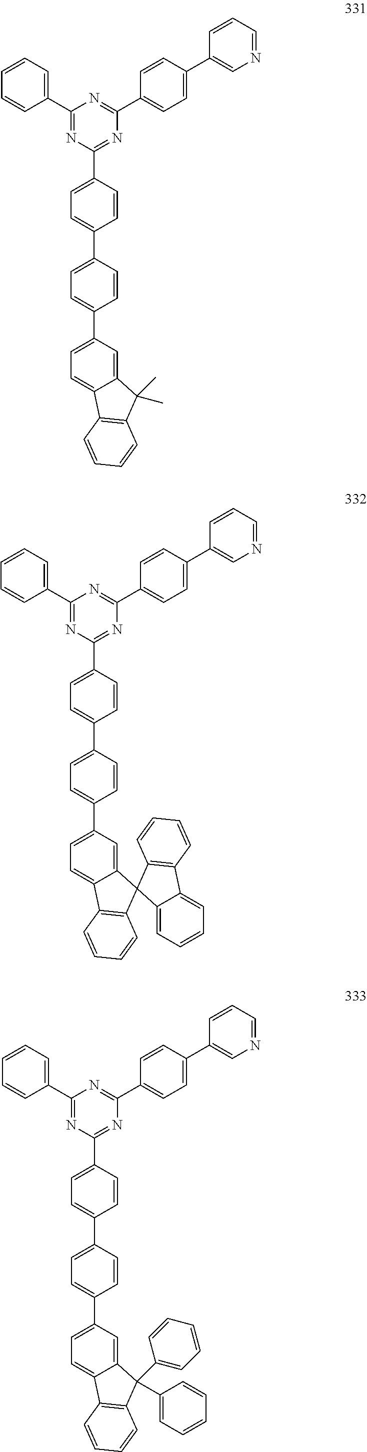 [SMILES: CC1(C)c2ccccc2-c2ccc(-c3ccc(-c4ccc(-c5nc(-c6ccccc6)nc(-c6ccc(-c7cccnc7)cc6)n5)cc4)cc3)cc21 ; c1ccc(-c2nc(-c3ccc(-c4ccc(-c5ccc6c(c5)C5(c7ccccc7-c7ccccc75)c5ccccc5-6)cc4)cc3)nc(-c3ccc(-c4cccnc4)cc3)n2)cc1 ; c1ccc(-c2nc(-c3ccc(-c4ccc(-c5ccc6c(c5)C(c5ccccc5)(c5ccccc5)c5ccccc5-6)cc4)cc3)nc(-c3ccc(-c4cccnc4)cc3)n2)cc1]

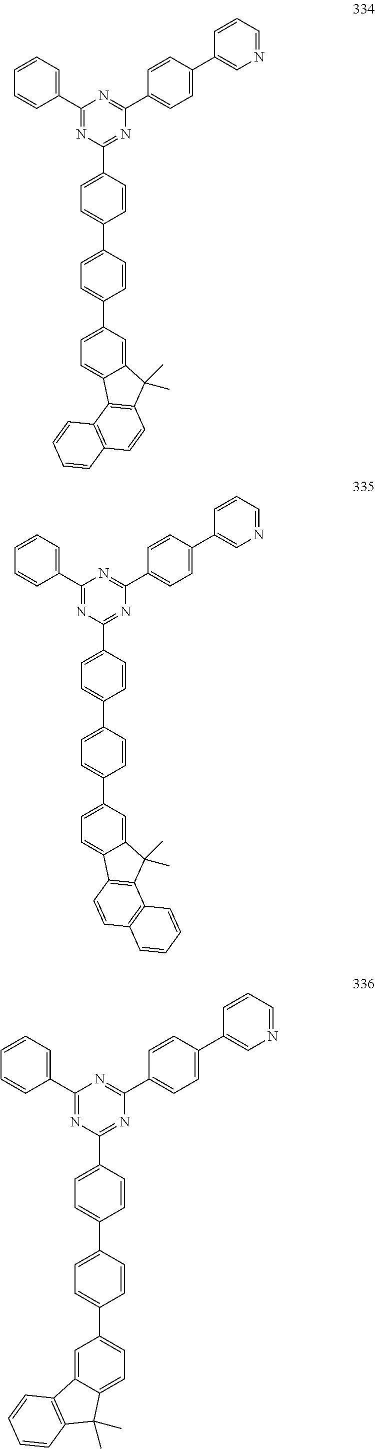 [SMILES: CC1(C)c2cc(-c3ccc(-c4ccc(-c5nc(-c6ccccc6)nc(-c6ccc(-c7cccnc7)cc6)n5)cc4)cc3)ccc2-c2c1ccc1ccccc21 ; CC1(C)c2cc(-c3ccc(-c4ccc(-c5nc(-c6ccccc6)nc(-c6ccc(-c7cccnc7)cc6)n5)cc4)cc3)ccc2-c2ccc3ccccc3c21 ; CC1(C)c2ccccc2-c2cc(-c3ccc(-c4ccc(-c5nc(-c6ccccc6)nc(-c6ccc(-c7cccnc7)cc6)n5)cc4)cc3)ccc21]

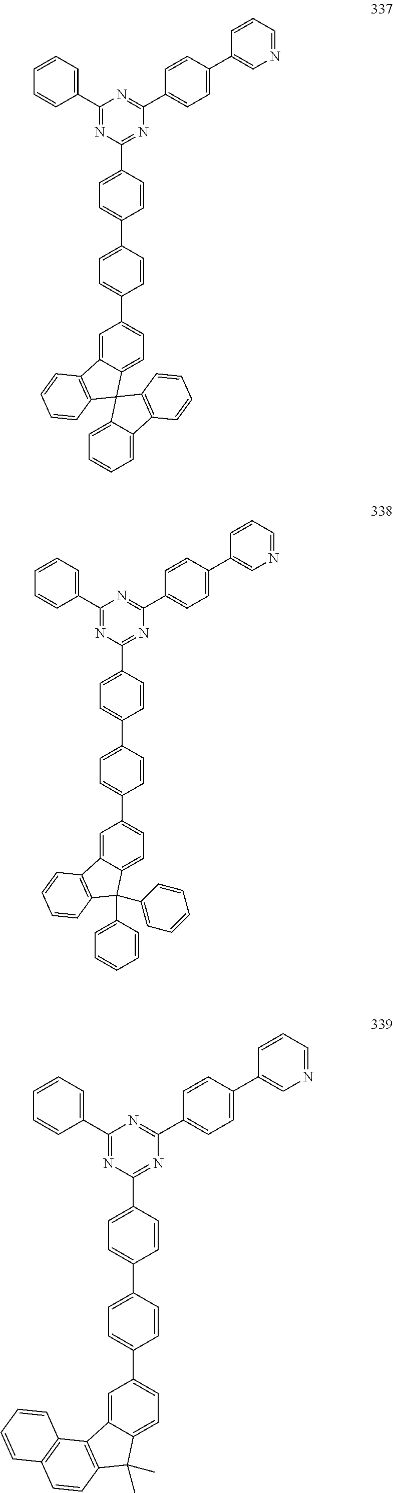 [SMILES: c1ccc(-c2nc(-c3ccc(-c4ccc(-c5ccc6c(c5)-c5ccccc5C65c6ccccc6-c6ccccc65)cc4)cc3)nc(-c3ccc(-c4cccnc4)cc3)n2)cc1 ; c1ccc(-c2nc(-c3ccc(-c4ccc(-c5ccc6c(c5)-c5ccccc5C6(c5ccccc5)c5ccccc5)cc4)cc3)nc(-c3ccc(-c4cccnc4)cc3)n2)cc1 ; CC1(C)c2ccc(-c3ccc(-c4ccc(-c5nc(-c6ccccc6)nc(-c6ccc(-c7cccnc7)cc6)n5)cc4)cc3)cc2-c2c1ccc1ccccc21]

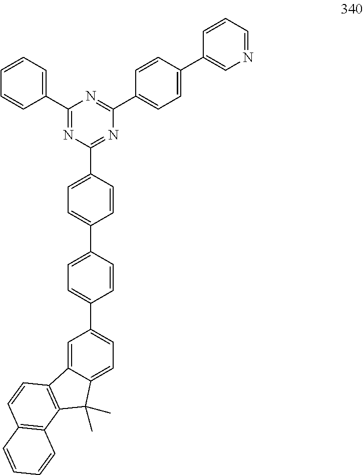 [SMILES: CC1(C)c2ccc(-c3ccc(-c4ccc(-c5nc(-c6ccccc6)nc(-c6ccc(-c7cccnc7)cc6)n5)cc4)cc3)cc2-c2ccc3ccccc3c21]

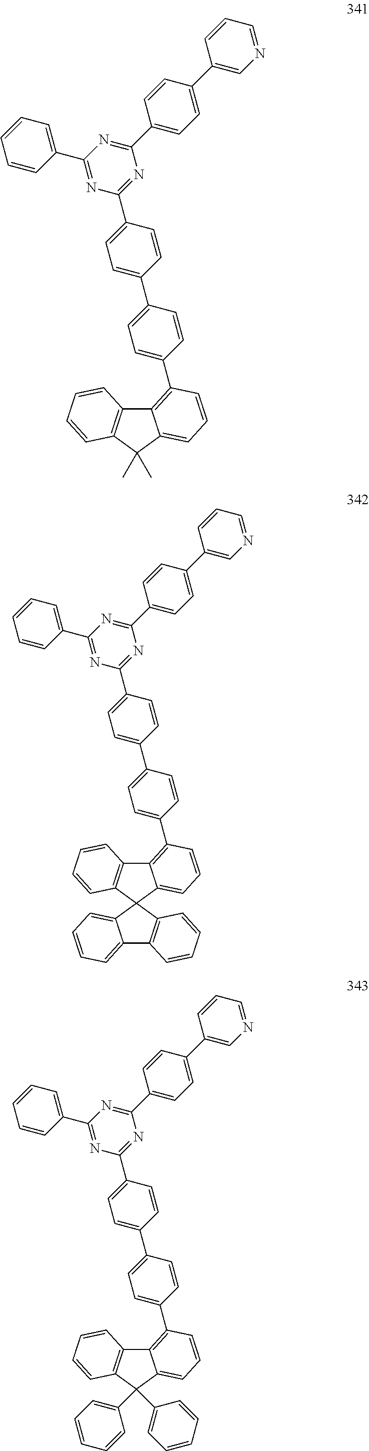 [SMILES: CC1(C)c2ccccc2-c2c(-c3ccc(-c4ccc(-c5nc(-c6ccccc6)nc(-c6ccc(-c7cccnc7)cc6)n5)cc4)cc3)cccc21 ; c1ccc(-c2nc(-c3ccc(-c4ccc(-c5cccc6c5-c5ccccc5C65c6ccccc6-c6ccccc65)cc4)cc3)nc(-c3ccc(-c4cccnc4)cc3)n2)cc1 ; c1ccc(-c2nc(-c3ccc(-c4ccc(-c5cccc6c5-c5ccccc5C6(c5ccccc5)c5ccccc5)cc4)cc3)nc(-c3ccc(-c4cccnc4)cc3)n2)cc1]

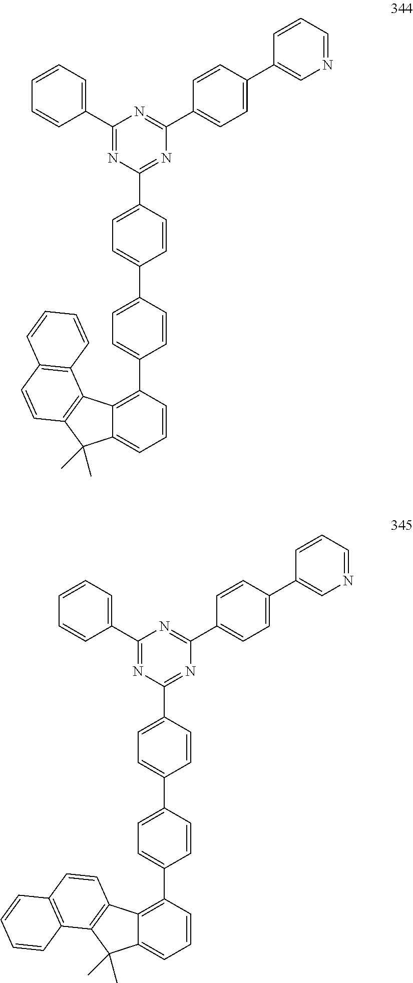 [SMILES: CC1(C)c2cccc(-c3ccc(-c4ccc(-c5nc(-c6ccccc6)nc(-c6ccc(-c7cccnc7)cc6)n5)cc4)cc3)c2-c2c1ccc1ccccc21 ; CC1(C)c2cccc(-c3ccc(-c4ccc(-c5nc(-c6ccccc6)nc(-c6ccc(-c7cccnc7)cc6)n5)cc4)cc3)c2-c2ccc3ccccc3c21]

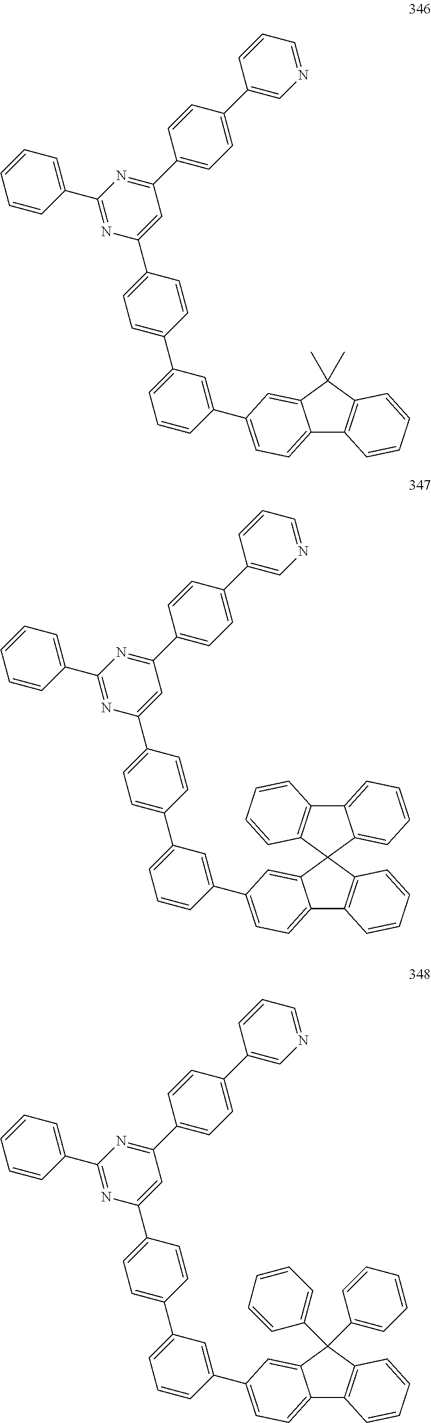 [SMILES: CC1(C)c2ccccc2-c2ccc(-c3cccc(-c4ccc(-c5cc(-c6ccc(-c7cccnc7)cc6)nc(-c6ccccc6)n5)cc4)c3)cc21 ; c1ccc(-c2nc(-c3ccc(-c4cccnc4)cc3)cc(-c3ccc(-c4cccc(-c5ccc6c(c5)C5(c7ccccc7-c7ccccc75)c5ccccc5-6)c4)cc3)n2)cc1 ; c1ccc(-c2nc(-c3ccc(-c4cccnc4)cc3)cc(-c3ccc(-c4cccc(-c5ccc6c(c5)C(c5ccccc5)(c5ccccc5)c5ccccc5-6)c4)cc3)n2)cc1]

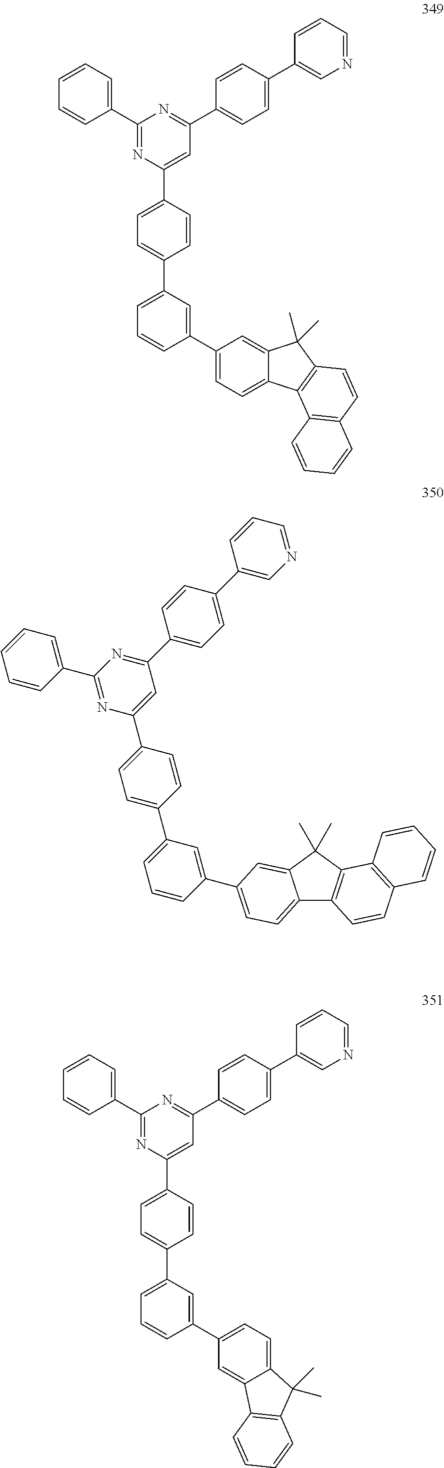 [SMILES: CC1(C)c2cc(-c3cccc(-c4ccc(-c5cc(-c6ccc(-c7cccnc7)cc6)nc(-c6ccccc6)n5)cc4)c3)ccc2-c2c1ccc1ccccc21 ; CC1(C)c2cc(-c3cccc(-c4ccc(-c5cc(-c6ccc(-c7cccnc7)cc6)nc(-c6ccccc6)n5)cc4)c3)ccc2-c2ccc3ccccc3c21 ; CC1(C)c2ccccc2-c2cc(-c3cccc(-c4ccc(-c5cc(-c6ccc(-c7cccnc7)cc6)nc(-c6ccccc6)n5)cc4)c3)ccc21]

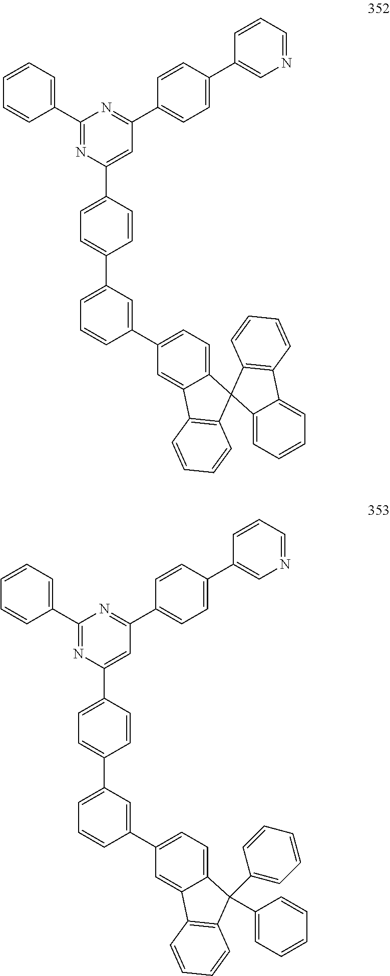 [SMILES: c1ccc(-c2nc(-c3ccc(-c4cccnc4)cc3)cc(-c3ccc(-c4cccc(-c5ccc6c(c5)-c5ccccc5C65c6ccccc6-c6ccccc65)c4)cc3)n2)cc1 ; c1ccc(-c2nc(-c3ccc(-c4cccnc4)cc3)cc(-c3ccc(-c4cccc(-c5ccc6c(c5)-c5ccccc5C6(c5ccccc5)c5ccccc5)c4)cc3)n2)cc1]

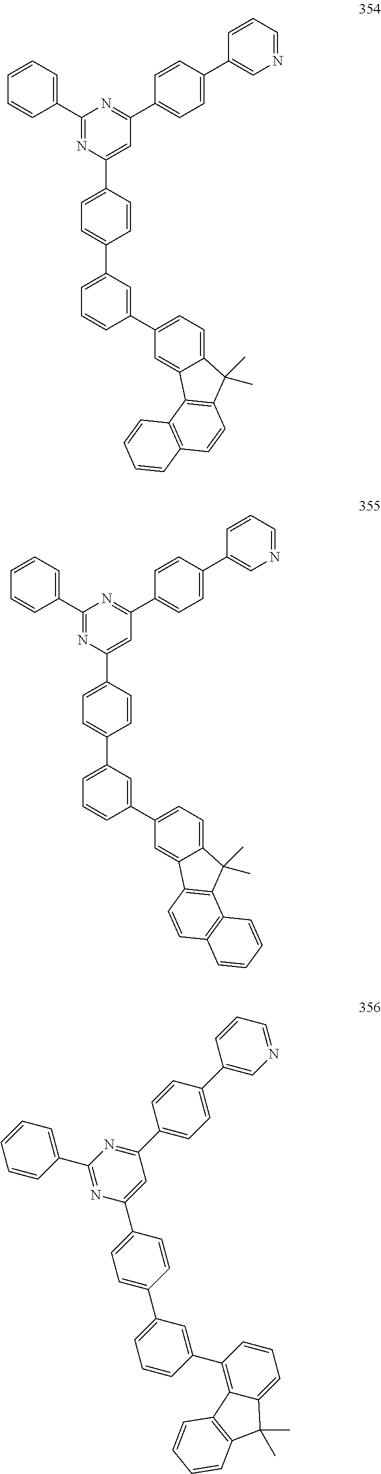 [SMILES: CC1(C)c2ccc(-c3cccc(-c4ccc(-c5cc(-c6ccc(-c7cccnc7)cc6)nc(-c6ccccc6)n5)cc4)c3)cc2-c2c1ccc1ccccc21 ; CC1(C)c2ccc(-c3cccc(-c4ccc(-c5cc(-c6ccc(-c7cccnc7)cc6)nc(-c6ccccc6)n5)cc4)c3)cc2-c2ccc3ccccc3c21 ; CC1(C)c2ccccc2-c2c(-c3cccc(-c4ccc(-c5cc(-c6ccc(-c7cccnc7)cc6)nc(-c6ccccc6)n5)cc4)c3)cccc21]

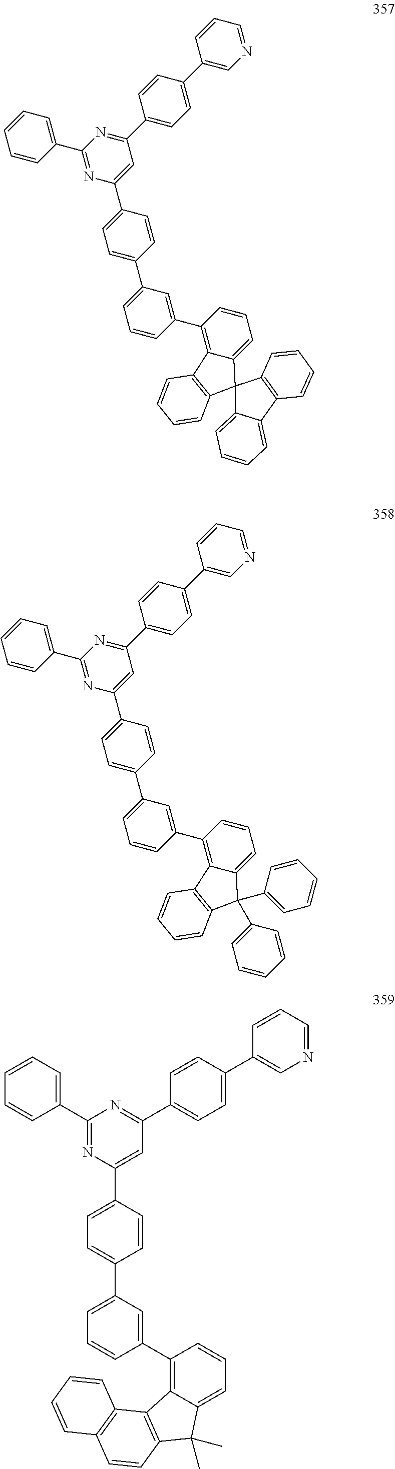

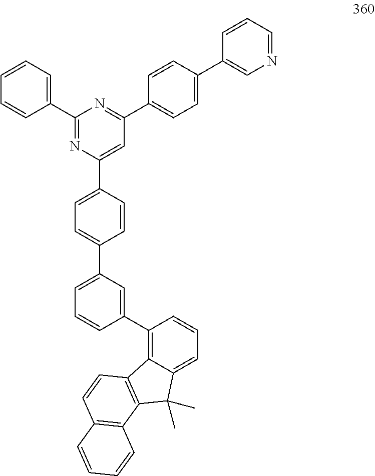 [SMILES: CC1(C)c2cccc(-c3cccc(-c4ccc(-c5cc(-c6ccc(-c7cccnc7)cc6)nc(-c6ccccc6)n5)cc4)c3)c2-c2ccc3ccccc3c21]

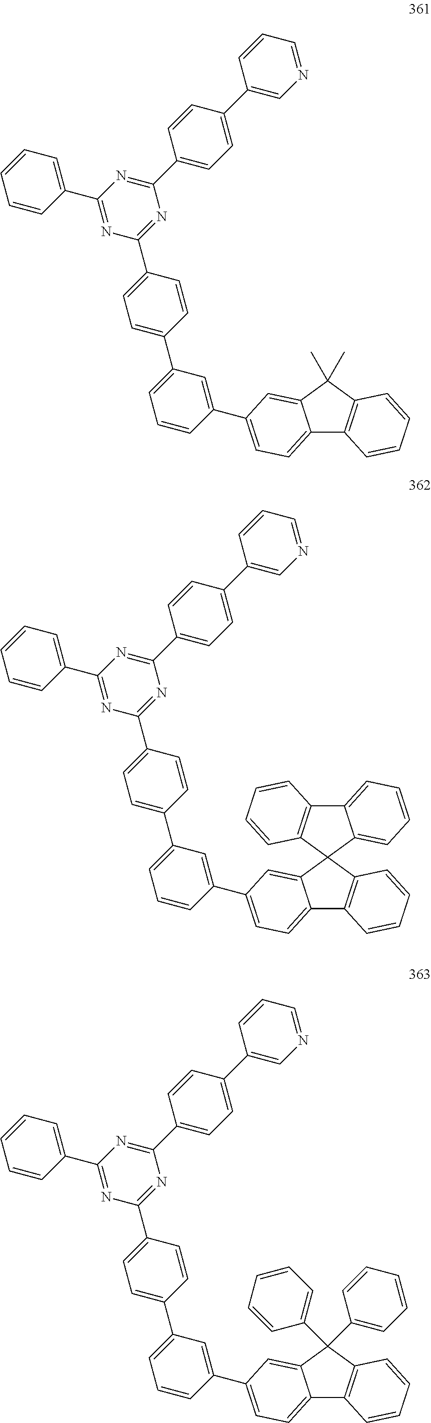 [SMILES: CC1(C)c2ccccc2-c2ccc(-c3cccc(-c4ccc(-c5nc(-c6ccccc6)nc(-c6ccc(-c7cccnc7)cc6)n5)cc4)c3)cc21 ; c1ccc(-c2nc(-c3ccc(-c4cccnc4)cc3)nc(-c3ccc(-c4cccc(-c5ccc6c(c5)C5(c7ccccc7-c7ccccc75)c5ccccc5-6)c4)cc3)n2)cc1 ; c1ccc(-c2nc(-c3ccc(-c4cccnc4)cc3)nc(-c3ccc(-c4cccc(-c5ccc6c(c5)C(c5ccccc5)(c5ccccc5)c5ccccc5-6)c4)cc3)n2)cc1]

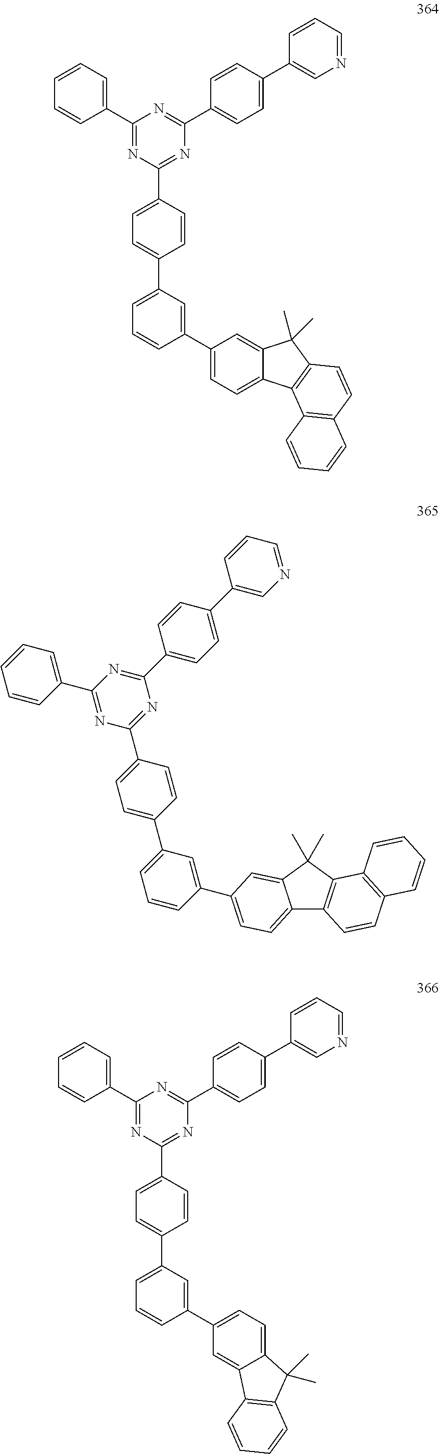 [SMILES: CC1(C)c2cc(-c3cccc(-c4ccc(-c5nc(-c6ccccc6)nc(-c6ccc(-c7cccnc7)cc6)n5)cc4)c3)ccc2-c2c1ccc1ccccc21 ; CC1(C)c2cc(-c3cccc(-c4ccc(-c5nc(-c6ccccc6)nc(-c6ccc(-c7cccnc7)cc6)n5)cc4)c3)ccc2-c2ccc3ccccc3c21 ; CC1(C)c2ccccc2-c2cc(-c3cccc(-c4ccc(-c5nc(-c6ccccc6)nc(-c6ccc(-c7cccnc7)cc6)n5)cc4)c3)ccc21]

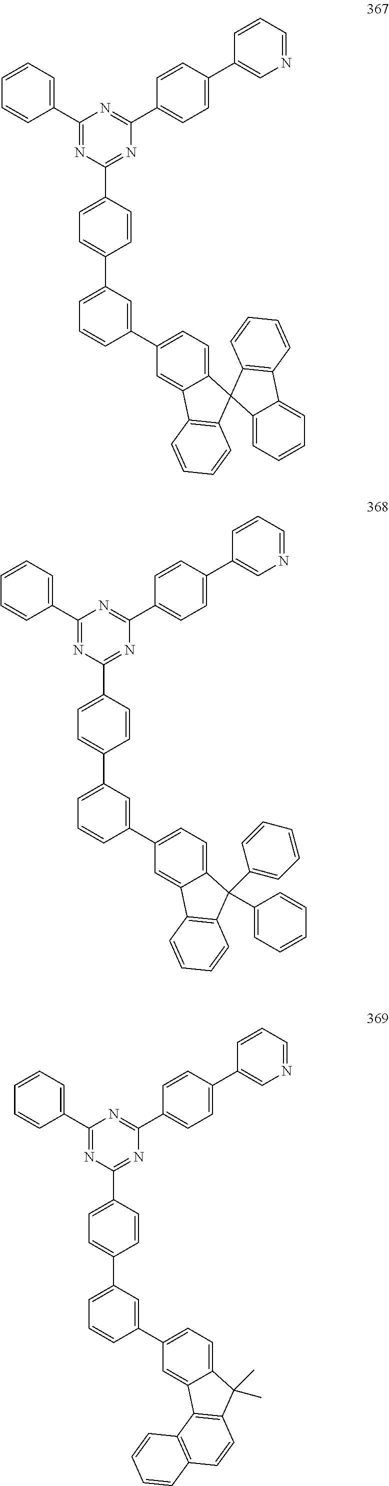 [SMILES: c1ccc(-c2nc(-c3ccc(-c4cccnc4)cc3)nc(-c3ccc(-c4cccc(-c5ccc6c(c5)-c5ccccc5C65c6ccccc6-c6ccccc65)c4)cc3)n2)cc1 ; c1ccc(-c2nc(-c3ccc(-c4cccnc4)cc3)nc(-c3ccc(-c4cccc(-c5ccc6c(c5)-c5ccccc5C6(c5ccccc5)c5ccccc5)c4)cc3)n2)cc1 ; CC1(C)c2ccc(-c3cccc(-c4ccc(-c5nc(-c6ccccc6)nc(-c6ccc(-c7cccnc7)cc6)n5)cc4)c3)cc2-c2c1ccc1ccccc21]

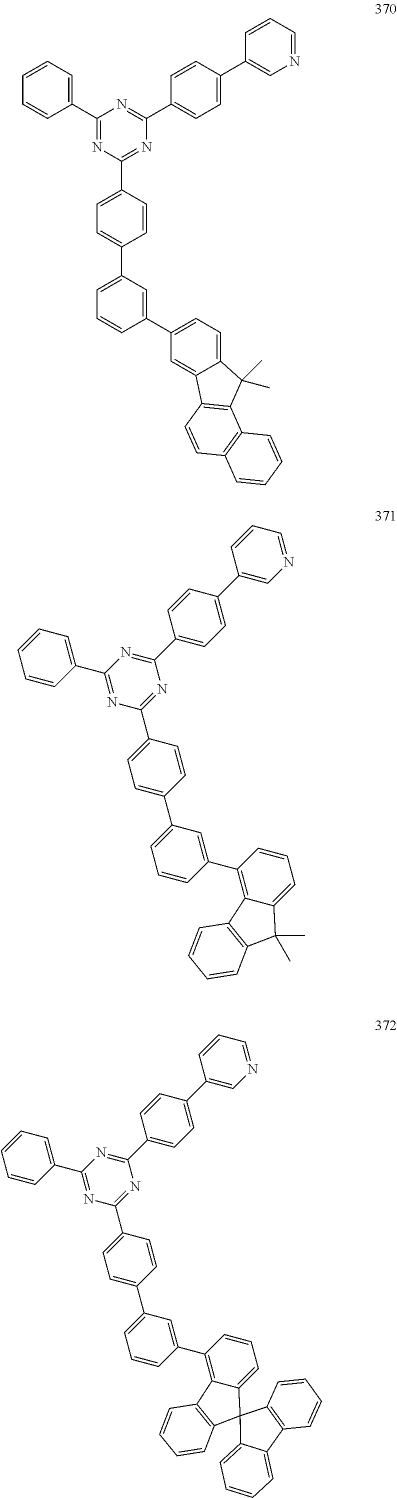 [SMILES: CC1(C)c2ccc(-c3cccc(-c4ccc(-c5nc(-c6ccccc6)nc(-c6ccc(-c7cccnc7)cc6)n5)cc4)c3)cc2-c2ccc3ccccc3c21 ; CC1(C)c2ccccc2-c2c(-c3cccc(-c4ccc(-c5nc(-c6ccccc6)nc(-c6ccc(-c7cccnc7)cc6)n5)cc4)c3)cccc21 ; c1ccc(-c2nc(-c3ccc(-c4cccnc4)cc3)nc(-c3ccc(-c4cccc(-c5cccc6c5-c5ccccc5C65c6ccccc6-c6ccccc65)c4)cc3)n2)cc1]

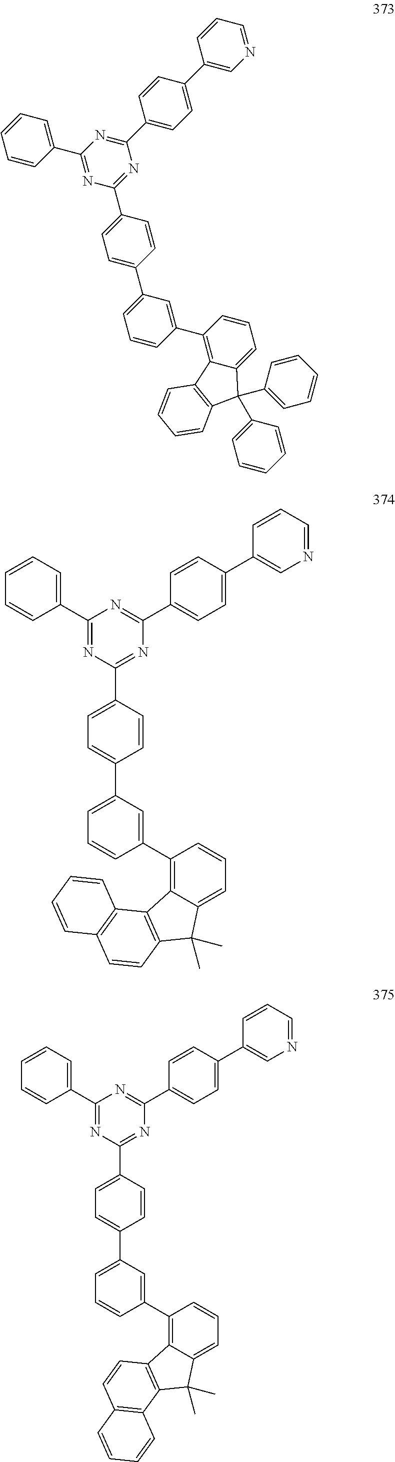

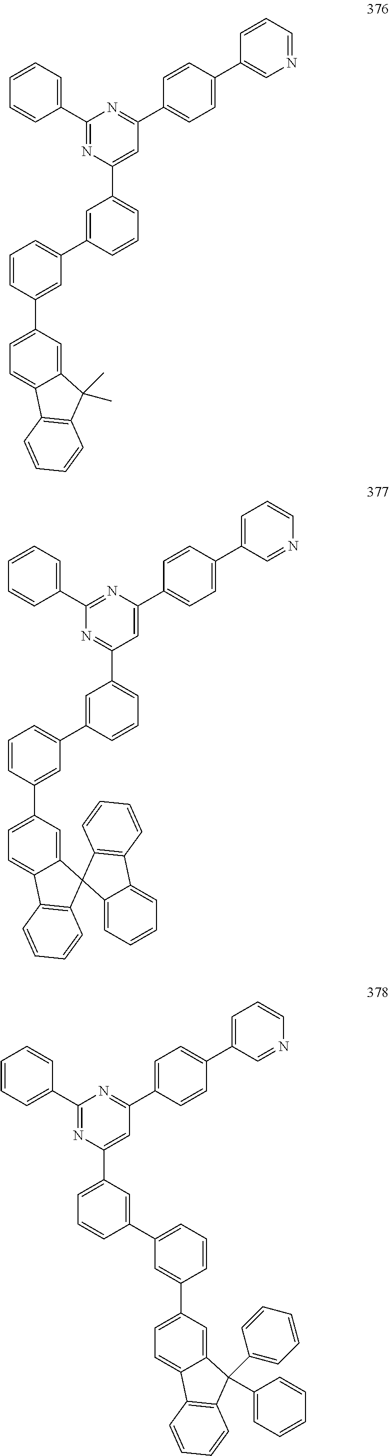 [SMILES: CC1(C)c2ccccc2-c2ccc(-c3cccc(-c4cccc(-c5cc(-c6ccc(-c7cccnc7)cc6)nc(-c6ccccc6)n5)c4)c3)cc21 ; c1ccc(-c2nc(-c3ccc(-c4cccnc4)cc3)cc(-c3cccc(-c4cccc(-c5ccc6c(c5)C5(c7ccccc7-c7ccccc75)c5ccccc5-6)c4)c3)n2)cc1 ; c1ccc(-c2nc(-c3ccc(-c4cccnc4)cc3)cc(-c3cccc(-c4cccc(-c5ccc6c(c5)C(c5ccccc5)(c5ccccc5)c5ccccc5-6)c4)c3)n2)cc1]

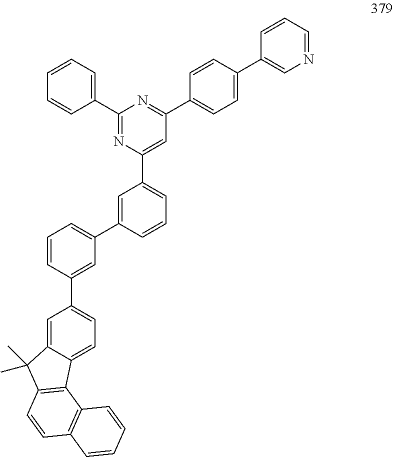 [SMILES: CC1(C)c2cc(-c3cccc(-c4cccc(-c5cc(-c6ccc(-c7cccnc7)cc6)nc(-c6ccccc6)n5)c4)c3)ccc2-c2c1ccc1ccccc21]

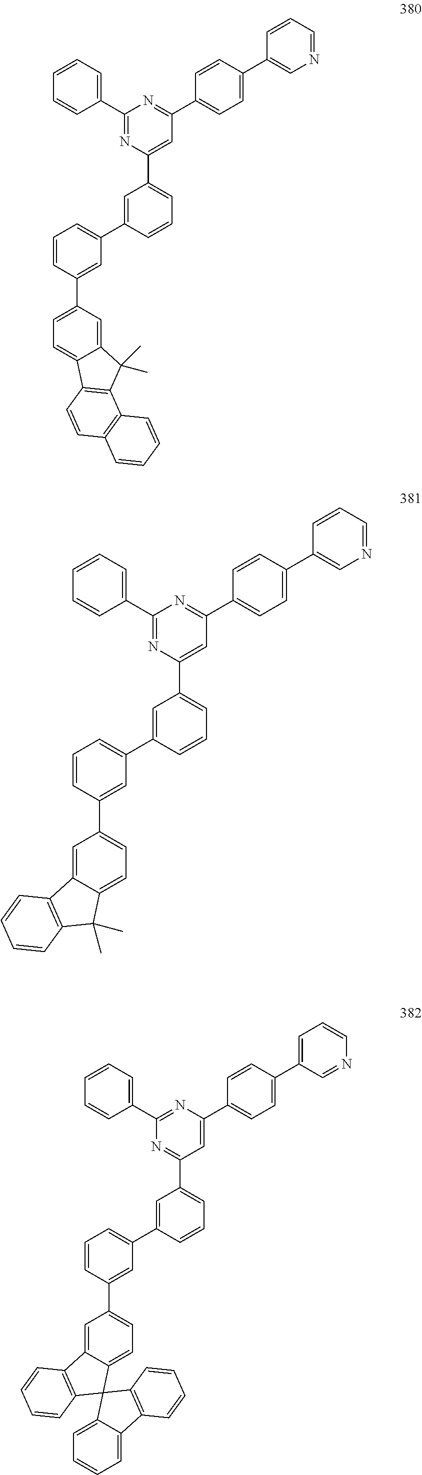 [SMILES: CC1(C)c2cc(-c3cccc(-c4cccc(-c5cc(-c6ccc(-c7cccnc7)cc6)nc(-c6ccccc6)n5)c4)c3)ccc2-c2ccc3ccccc3c21 ; CC1(C)c2ccccc2-c2cc(-c3cccc(-c4cccc(-c5cc(-c6ccc(-c7cccnc7)cc6)nc(-c6ccccc6)n5)c4)c3)ccc21 ; c1ccc(-c2nc(-c3ccc(-c4cccnc4)cc3)cc(-c3cccc(-c4cccc(-c5ccc6c(c5)-c5ccccc5C65c6ccccc6-c6ccccc65)c4)c3)n2)cc1]

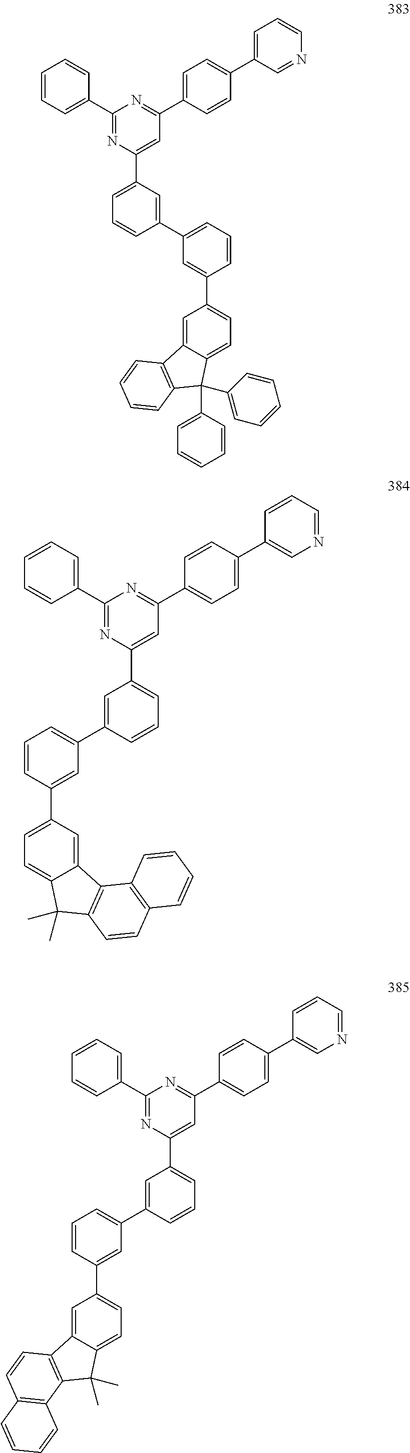 [SMILES: c1ccc(-c2nc(-c3ccc(-c4cccnc4)cc3)cc(-c3cccc(-c4cccc(-c5ccc6c(c5)-c5ccccc5C6(c5ccccc5)c5ccccc5)c4)c3)n2)cc1 ; CC1(C)c2ccc(-c3cccc(-c4cccc(-c5cc(-c6ccc(-c7cccnc7)cc6)nc(-c6ccccc6)n5)c4)c3)cc2-c2c1ccc1ccccc21 ; CC1(C)c2ccc(-c3cccc(-c4cccc(-c5cc(-c6ccc(-c7cccnc7)cc6)nc(-c6ccccc6)n5)c4)c3)cc2-c2ccc3ccccc3c21]

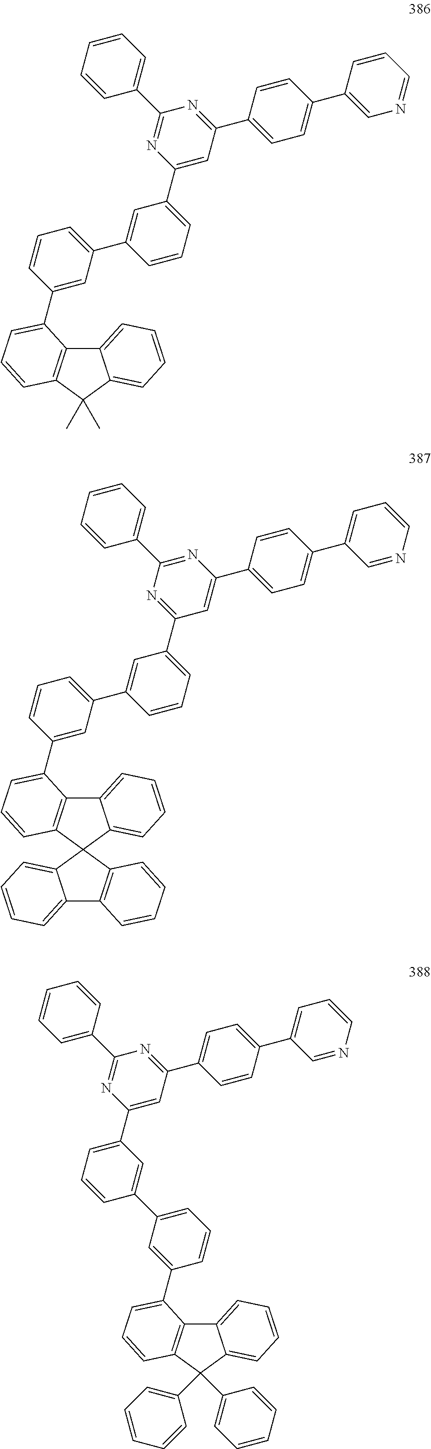 [SMILES: CC1(C)c2ccccc2-c2c(-c3cccc(-c4cccc(-c5cc(-c6ccc(-c7cccnc7)cc6)nc(-c6ccccc6)n5)c4)c3)cccc21 ; c1ccc(-c2nc(-c3ccc(-c4cccnc4)cc3)cc(-c3cccc(-c4cccc(-c5cccc6c5-c5ccccc5C65c6ccccc6-c6ccccc65)c4)c3)n2)cc1 ; c1ccc(-c2nc(-c3ccc(-c4cccnc4)cc3)cc(-c3cccc(-c4cccc(-c5cccc6c5-c5ccccc5C6(c5ccccc5)c5ccccc5)c4)c3)n2)cc1]

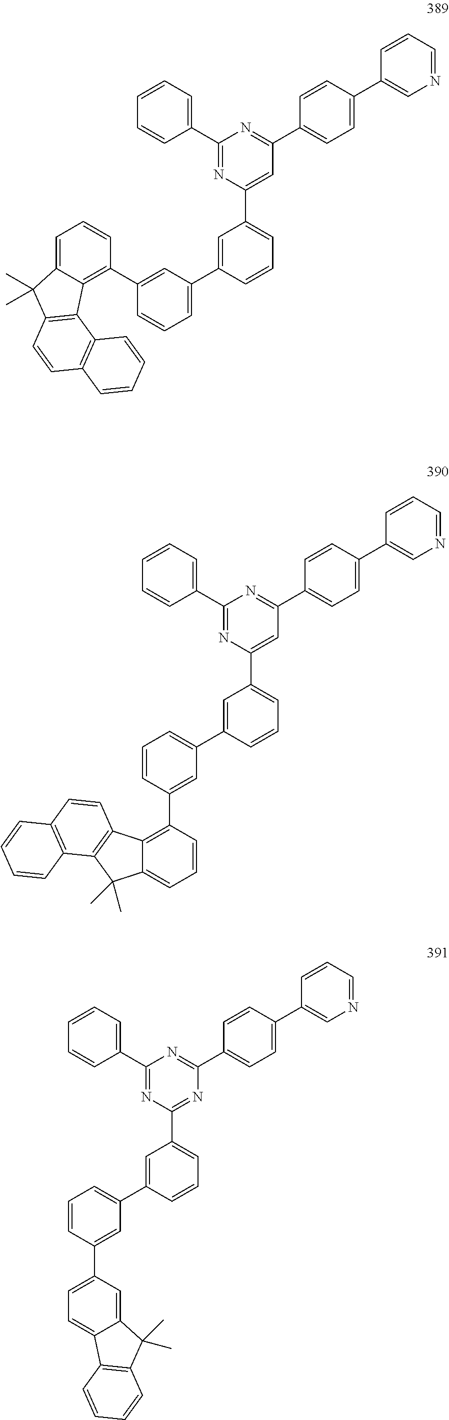 [SMILES: CC1(C)c2cccc(-c3cccc(-c4cccc(-c5cc(-c6ccc(-c7cccnc7)cc6)nc(-c6ccccc6)n5)c4)c3)c2-c2c1ccc1ccccc21 ; CC1(C)c2cccc(-c3cccc(-c4cccc(-c5cc(-c6ccc(-c7cccnc7)cc6)nc(-c6ccccc6)n5)c4)c3)c2-c2ccc3ccccc3c21 ; CC1(C)c2ccccc2-c2ccc(-c3cccc(-c4cccc(-c5nc(-c6ccccc6)nc(-c6ccc(-c7cccnc7)cc6)n5)c4)c3)cc21]

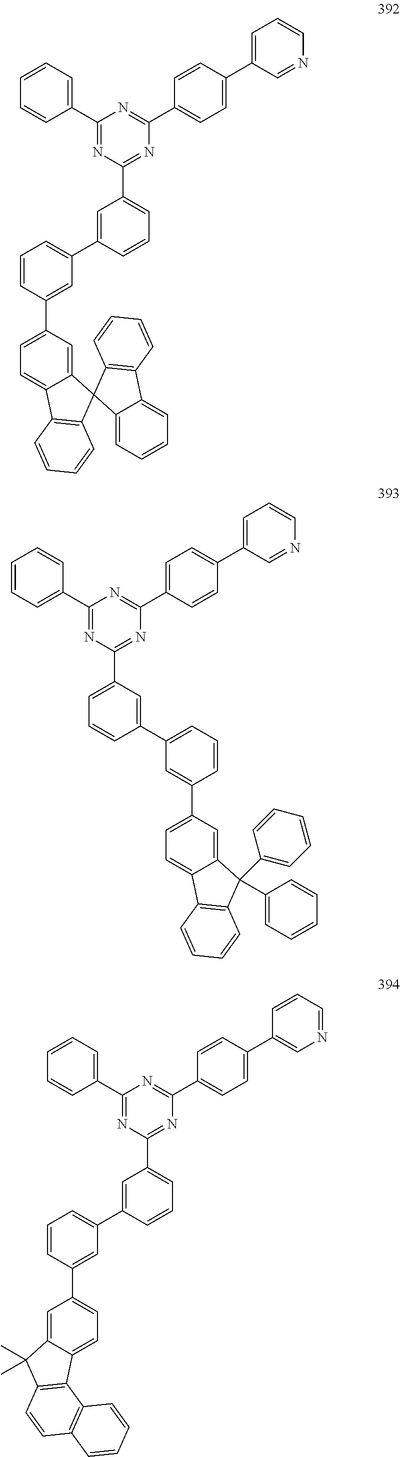 [SMILES: c1ccc(-c2nc(-c3ccc(-c4cccnc4)cc3)nc(-c3cccc(-c4cccc(-c5ccc6c(c5)C5(c7ccccc7-c7ccccc75)c5ccccc5-6)c4)c3)n2)cc1 ; c1ccc(-c2nc(-c3ccc(-c4cccnc4)cc3)nc(-c3cccc(-c4cccc(-c5ccc6c(c5)C(c5ccccc5)(c5ccccc5)c5ccccc5-6)c4)c3)n2)cc1 ; CC1(C)c2cc(-c3cccc(-c4cccc(-c5nc(-c6ccccc6)nc(-c6ccc(-c7cccnc7)cc6)n5)c4)c3)ccc2-c2c1ccc1ccccc21]

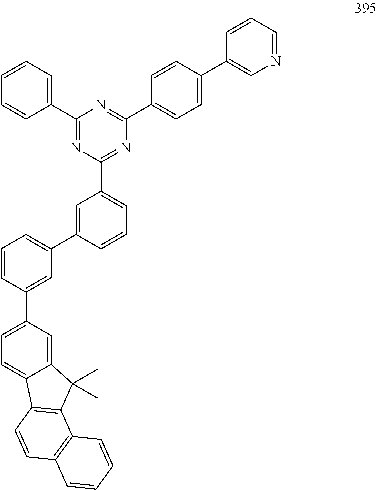 [SMILES: CC1(C)c2cc(-c3cccc(-c4cccc(-c5nc(-c6ccccc6)nc(-c6ccc(-c7cccnc7)cc6)n5)c4)c3)ccc2-c2ccc3ccccc3c21]

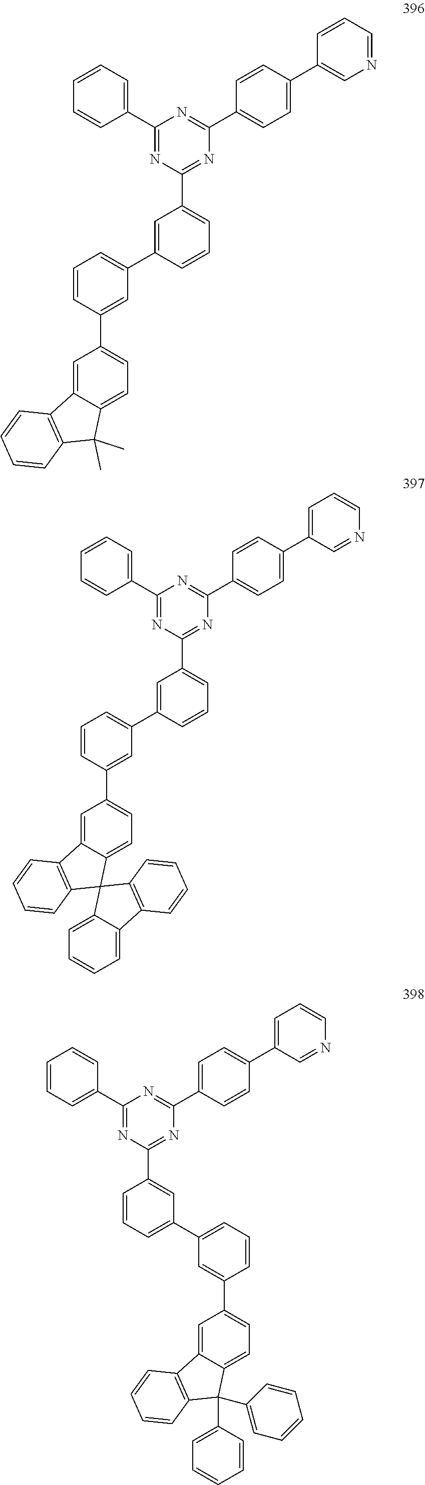 [SMILES: CC1(C)c2ccccc2-c2cc(-c3cccc(-c4cccc(-c5nc(-c6ccccc6)nc(-c6ccc(-c7cccnc7)cc6)n5)c4)c3)ccc21 ; c1ccc(-c2nc(-c3ccc(-c4cccnc4)cc3)nc(-c3cccc(-c4cccc(-c5ccc6c(c5)-c5ccccc5C65c6ccccc6-c6ccccc65)c4)c3)n2)cc1 ; c1ccc(-c2nc(-c3ccc(-c4cccnc4)cc3)nc(-c3cccc(-c4cccc(-c5ccc6c(c5)-c5ccccc5C6(c5ccccc5)c5ccccc5)c4)c3)n2)cc1]

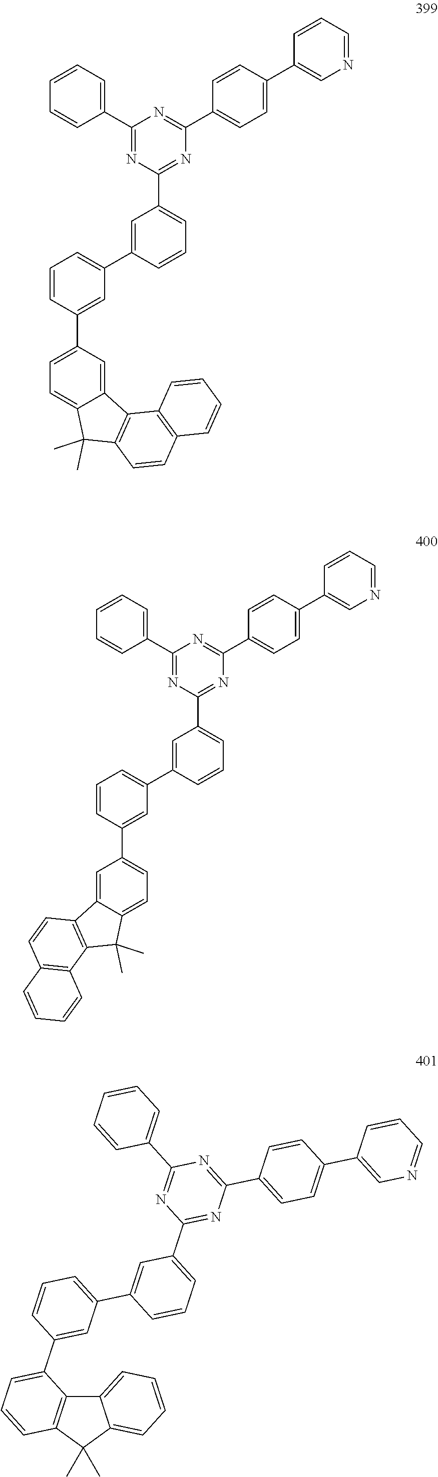 [SMILES: CC1(C)c2ccc(-c3cccc(-c4cccc(-c5nc(-c6ccccc6)nc(-c6ccc(-c7cccnc7)cc6)n5)c4)c3)cc2-c2c1ccc1ccccc21 ; CC1(C)c2ccc(-c3cccc(-c4cccc(-c5nc(-c6ccccc6)nc(-c6ccc(-c7cccnc7)cc6)n5)c4)c3)cc2-c2ccc3ccccc3c21 ; CC1(C)c2ccccc2-c2c(-c3cccc(-c4cccc(-c5nc(-c6ccccc6)nc(-c6ccc(-c7cccnc7)cc6)n5)c4)c3)cccc21]

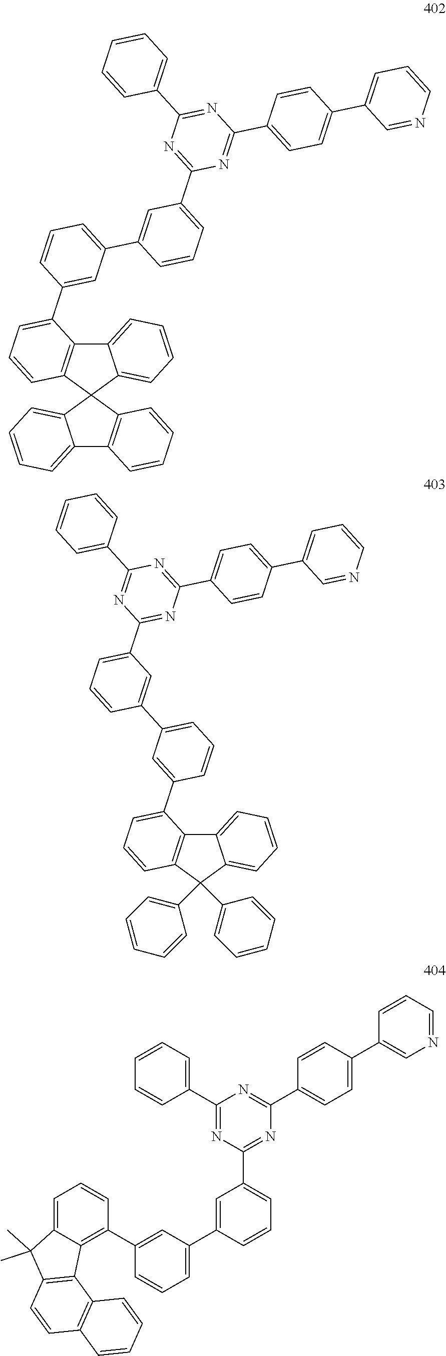

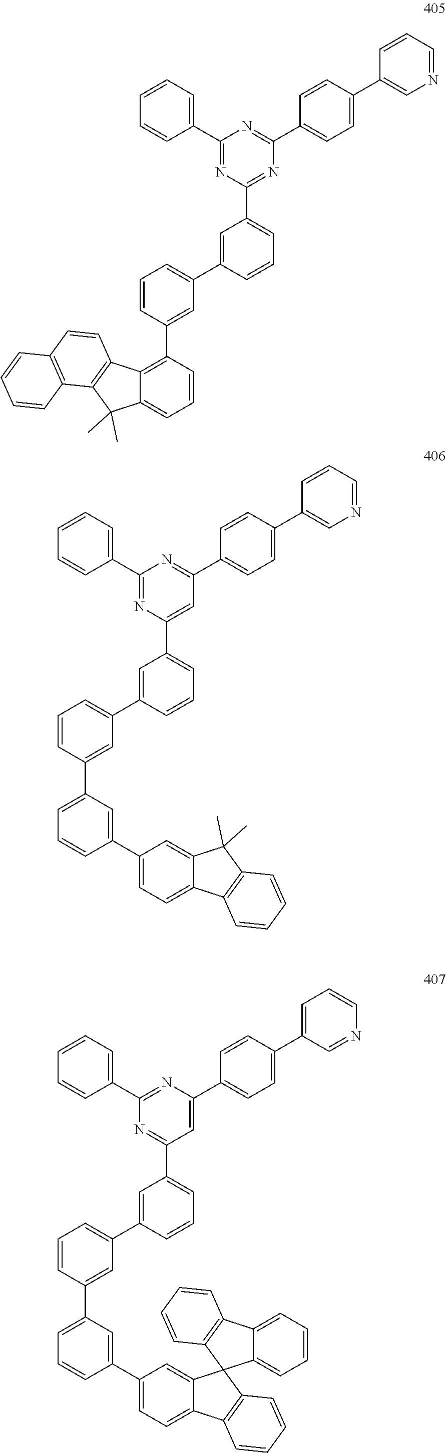 [SMILES: CC1(C)c2cccc(-c3cccc(-c4cccc(-c5nc(-c6ccccc6)nc(-c6ccc(-c7cccnc7)cc6)n5)c4)c3)c2-c2ccc3ccccc3c21 ; CC1(C)c2ccccc2-c2ccc(-c3cccc(-c4cccc(-c5cccc(-c6cc(-c7ccc(-c8cccnc8)cc7)nc(-c7ccccc7)n6)c5)c4)c3)cc21 ; c1ccc(-c2nc(-c3ccc(-c4cccnc4)cc3)cc(-c3cccc(-c4cccc(-c5cccc(-c6ccc7c(c6)C6(c8ccccc8-c8ccccc86)c6ccccc6-7)c5)c4)c3)n2)cc1]

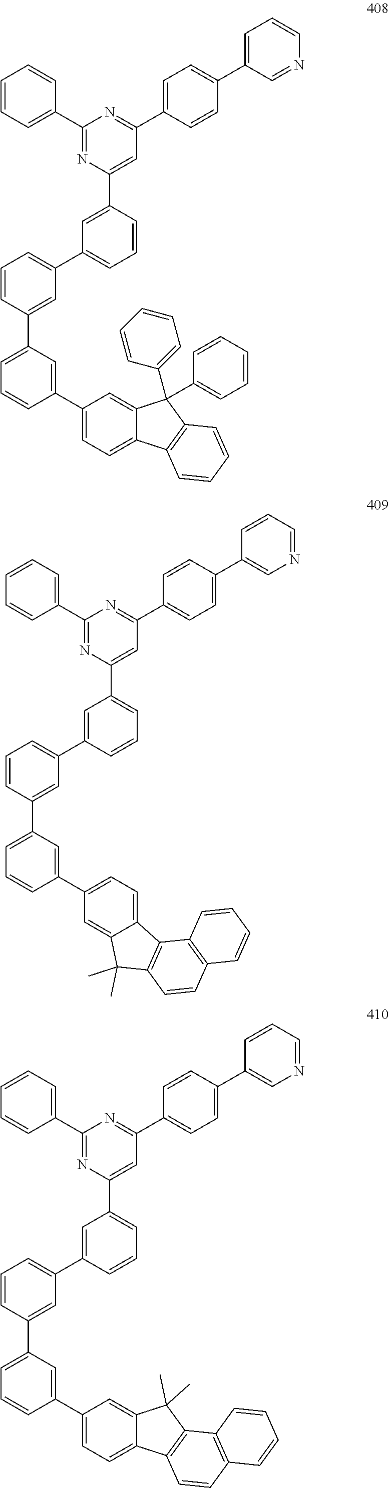 [SMILES: c1ccc(-c2nc(-c3ccc(-c4cccnc4)cc3)cc(-c3cccc(-c4cccc(-c5cccc(-c6ccc7c(c6)C(c6ccccc6)(c6ccccc6)c6ccccc6-7)c5)c4)c3)n2)cc1 ; CC1(C)c2cc(-c3cccc(-c4cccc(-c5cccc(-c6cc(-c7ccc(-c8cccnc8)cc7)nc(-c7ccccc7)n6)c5)c4)c3)ccc2-c2c1ccc1ccccc21 ; CC1(C)c2cc(-c3cccc(-c4cccc(-c5cccc(-c6cc(-c7ccc(-c8cccnc8)cc7)nc(-c7ccccc7)n6)c5)c4)c3)ccc2-c2ccc3ccccc3c21]

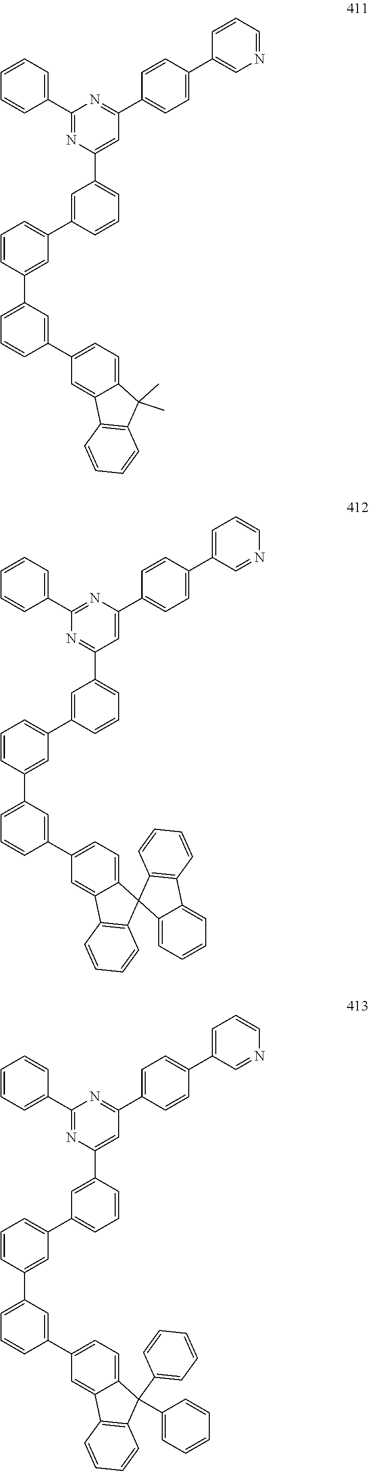 [SMILES: CC1(C)c2ccccc2-c2cc(-c3cccc(-c4cccc(-c5cccc(-c6cc(-c7ccc(-c8cccnc8)cc7)nc(-c7ccccc7)n6)c5)c4)c3)ccc21 ; c1ccc(-c2nc(-c3ccc(-c4cccnc4)cc3)cc(-c3cccc(-c4cccc(-c5cccc(-c6ccc7c(c6)-c6ccccc6C76c7ccccc7-c7ccccc76)c5)c4)c3)n2)cc1 ; c1ccc(-c2nc(-c3ccc(-c4cccnc4)cc3)cc(-c3cccc(-c4cccc(-c5cccc(-c6ccc7c(c6)-c6ccccc6C7(c6ccccc6)c6ccccc6)c5)c4)c3)n2)cc1]

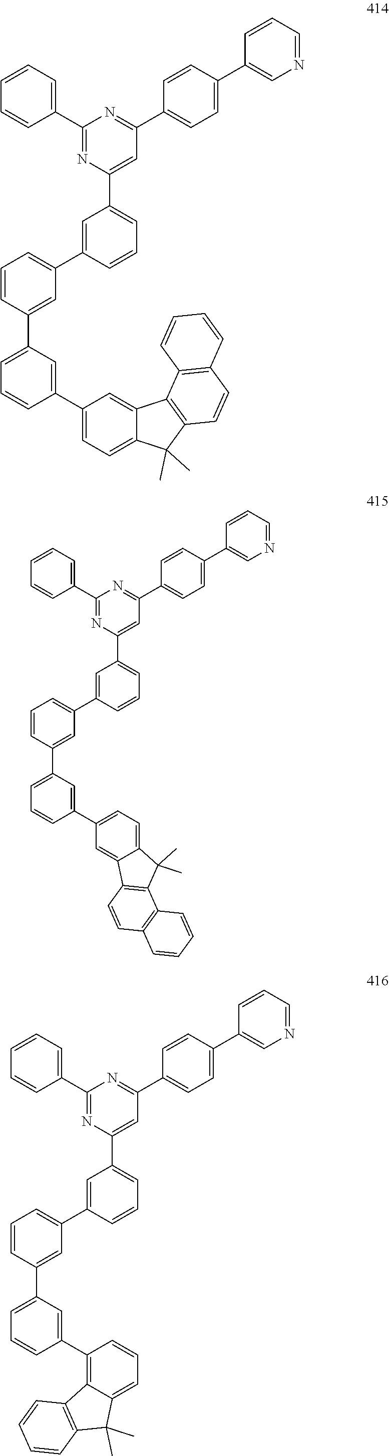 [SMILES: CC1(C)c2ccc(-c3cccc(-c4cccc(-c5cccc(-c6cc(-c7ccc(-c8cccnc8)cc7)nc(-c7ccccc7)n6)c5)c4)c3)cc2-c2c1ccc1ccccc21 ; CC1(C)c2ccc(-c3cccc(-c4cccc(-c5cccc(-c6cc(-c7ccc(-c8cccnc8)cc7)nc(-c7ccccc7)n6)c5)c4)c3)cc2-c2ccc3ccccc3c21 ; CC1(C)c2ccccc2-c2c(-c3cccc(-c4cccc(-c5cccc(-c6cc(-c7ccc(-c8cccnc8)cc7)nc(-c7ccccc7)n6)c5)c4)c3)cccc21]

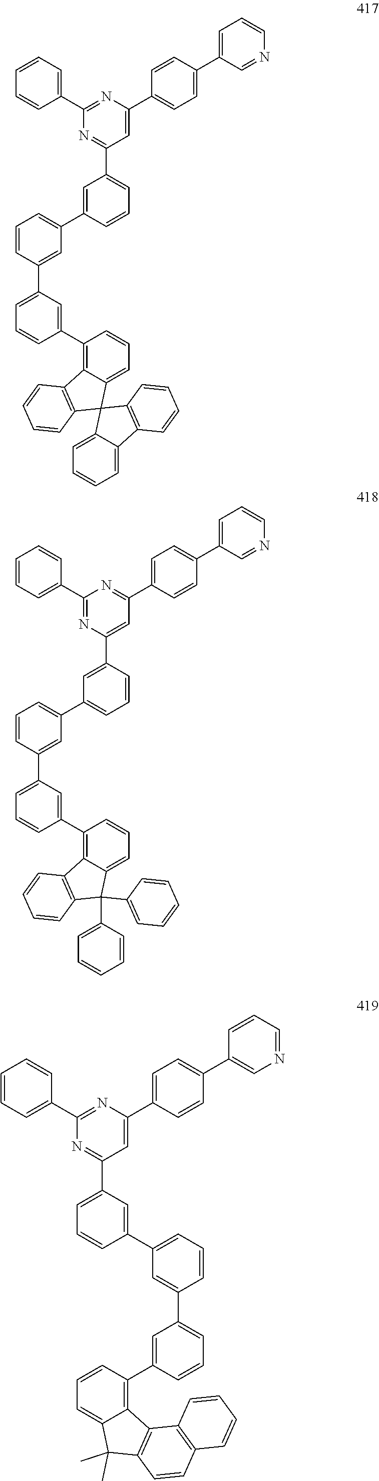 [SMILES: c1ccc(-c2nc(-c3ccc(-c4cccnc4)cc3)cc(-c3cccc(-c4cccc(-c5cccc(-c6cccc7c6-c6ccccc6C76c7ccccc7-c7ccccc76)c5)c4)c3)n2)cc1 ; c1ccc(-c2nc(-c3ccc(-c4cccnc4)cc3)cc(-c3cccc(-c4cccc(-c5cccc(-c6cccc7c6-c6ccccc6C7(c6ccccc6)c6ccccc6)c5)c4)c3)n2)cc1 ; CC1(C)c2cccc(-c3cccc(-c4cccc(-c5cccc(-c6cc(-c7ccc(-c8cccnc8)cc7)nc(-c7ccccc7)n6)c5)c4)c3)c2-c2c1ccc1ccccc21]

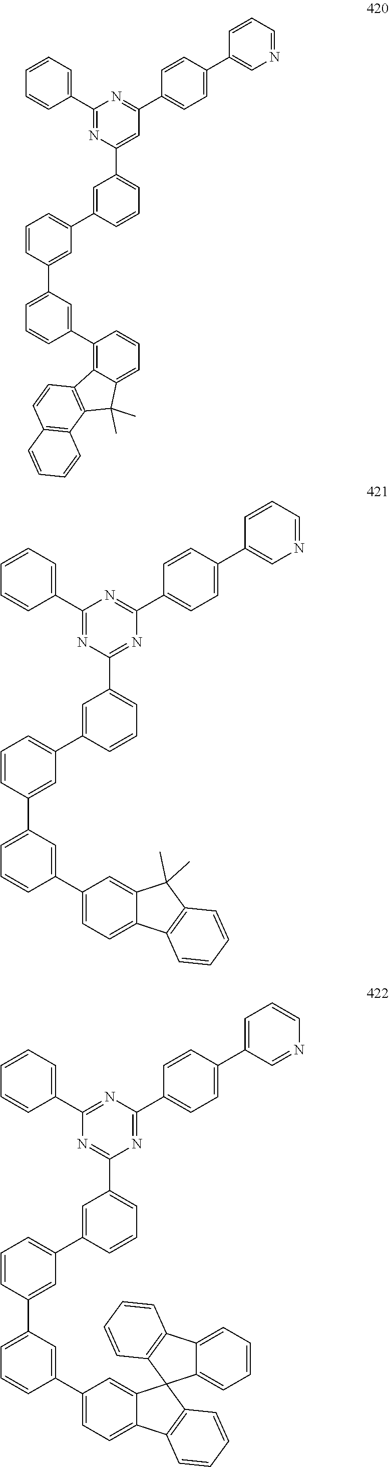 [SMILES: CC1(C)c2cccc(-c3cccc(-c4cccc(-c5cccc(-c6cc(-c7ccc(-c8cccnc8)cc7)nc(-c7ccccc7)n6)c5)c4)c3)c2-c2ccc3ccccc3c21 ; CC1(C)c2ccccc2-c2ccc(-c3cccc(-c4cccc(-c5cccc(-c6nc(-c7ccccc7)nc(-c7ccc(-c8cccnc8)cc7)n6)c5)c4)c3)cc21 ; c1ccc(-c2nc(-c3ccc(-c4cccnc4)cc3)nc(-c3cccc(-c4cccc(-c5cccc(-c6ccc7c(c6)C6(c8ccccc8-c8ccccc86)c6ccccc6-7)c5)c4)c3)n2)cc1]

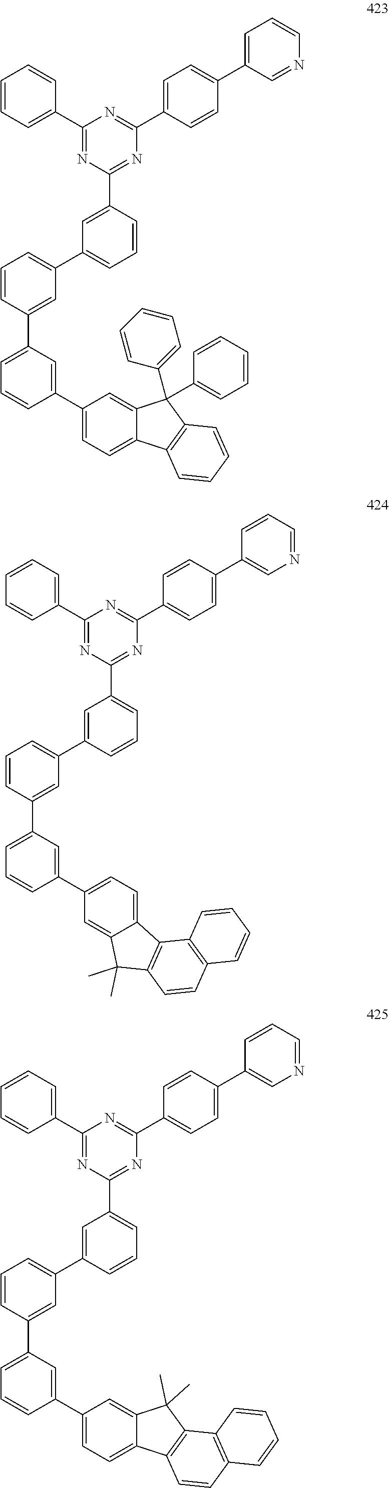 [SMILES: c1ccc(-c2nc(-c3ccc(-c4cccnc4)cc3)nc(-c3cccc(-c4cccc(-c5cccc(-c6ccc7c(c6)C(c6ccccc6)(c6ccccc6)c6ccccc6-7)c5)c4)c3)n2)cc1 ; CC1(C)c2cc(-c3cccc(-c4cccc(-c5cccc(-c6nc(-c7ccccc7)nc(-c7ccc(-c8cccnc8)cc7)n6)c5)c4)c3)ccc2-c2c1ccc1ccccc21 ; CC1(C)c2cc(-c3cccc(-c4cccc(-c5cccc(-c6nc(-c7ccccc7)nc(-c7ccc(-c8cccnc8)cc7)n6)c5)c4)c3)ccc2-c2ccc3ccccc3c21]

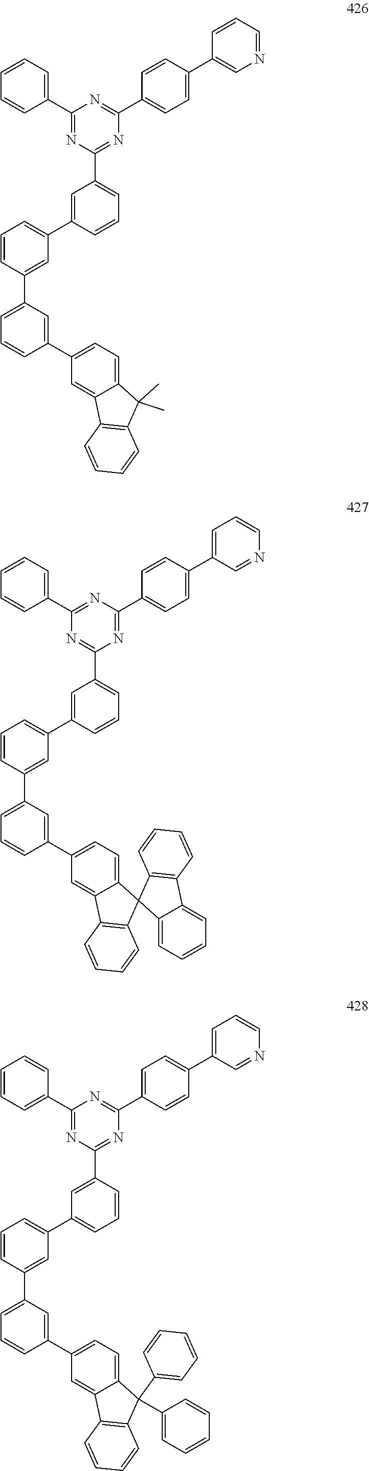 [SMILES: CC1(C)c2ccccc2-c2cc(-c3cccc(-c4cccc(-c5cccc(-c6nc(-c7ccccc7)nc(-c7ccc(-c8cccnc8)cc7)n6)c5)c4)c3)ccc21 ; c1ccc(-c2nc(-c3ccc(-c4cccnc4)cc3)nc(-c3cccc(-c4cccc(-c5cccc(-c6ccc7c(c6)-c6ccccc6C76c7ccccc7-c7ccccc76)c5)c4)c3)n2)cc1 ; c1ccc(-c2nc(-c3ccc(-c4cccnc4)cc3)nc(-c3cccc(-c4cccc(-c5cccc(-c6ccc7c(c6)-c6ccccc6C7(c6ccccc6)c6ccccc6)c5)c4)c3)n2)cc1]

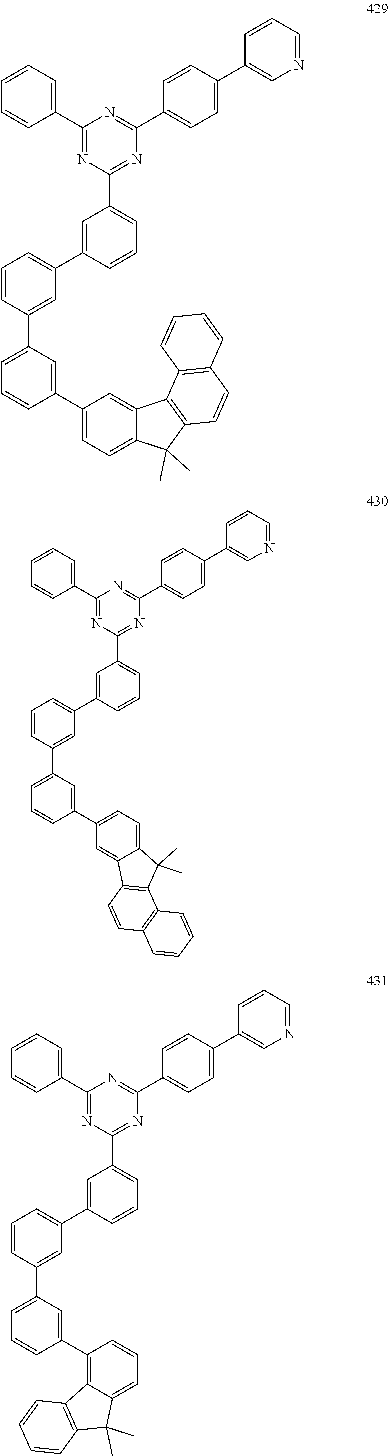 [SMILES: CC1(C)c2ccc(-c3cccc(-c4cccc(-c5cccc(-c6nc(-c7ccccc7)nc(-c7ccc(-c8cccnc8)cc7)n6)c5)c4)c3)cc2-c2c1ccc1ccccc21 ; CC1(C)c2ccc(-c3cccc(-c4cccc(-c5cccc(-c6nc(-c7ccccc7)nc(-c7ccc(-c8cccnc8)cc7)n6)c5)c4)c3)cc2-c2ccc3ccccc3c21 ; CC1(C)c2ccccc2-c2c(-c3cccc(-c4cccc(-c5cccc(-c6nc(-c7ccccc7)nc(-c7ccc(-c8cccnc8)cc7)n6)c5)c4)c3)cccc21]

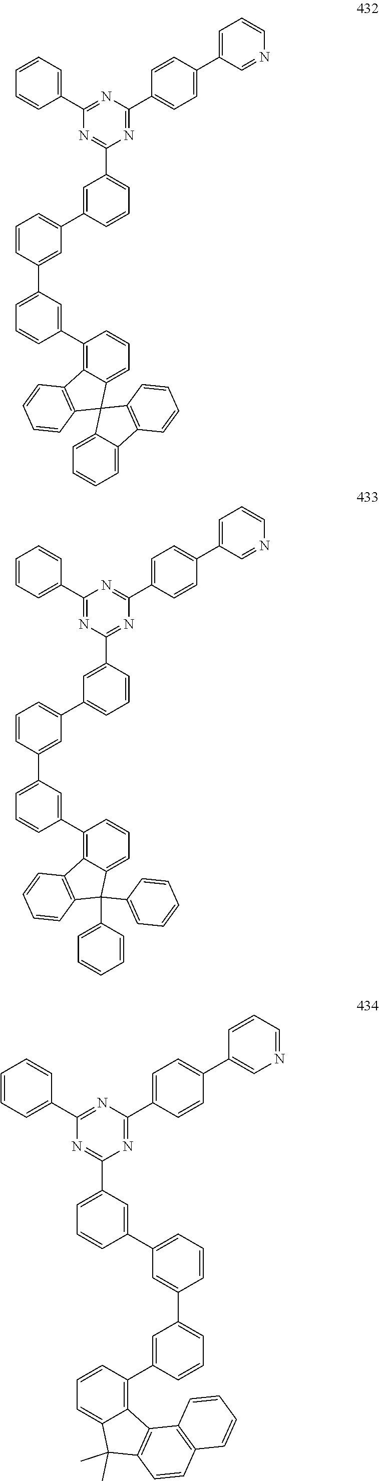 [SMILES: c1ccc(-c2nc(-c3ccc(-c4cccnc4)cc3)nc(-c3cccc(-c4cccc(-c5cccc(-c6cccc7c6-c6ccccc6C76c7ccccc7-c7ccccc76)c5)c4)c3)n2)cc1 ; c1ccc(-c2nc(-c3ccc(-c4cccnc4)cc3)nc(-c3cccc(-c4cccc(-c5cccc(-c6cccc7c6-c6ccccc6C7(c6ccccc6)c6ccccc6)c5)c4)c3)n2)cc1 ; CC1(C)c2cccc(-c3cccc(-c4cccc(-c5cccc(-c6nc(-c7ccccc7)nc(-c7ccc(-c8cccnc8)cc7)n6)c5)c4)c3)c2-c2c1ccc1ccccc21]

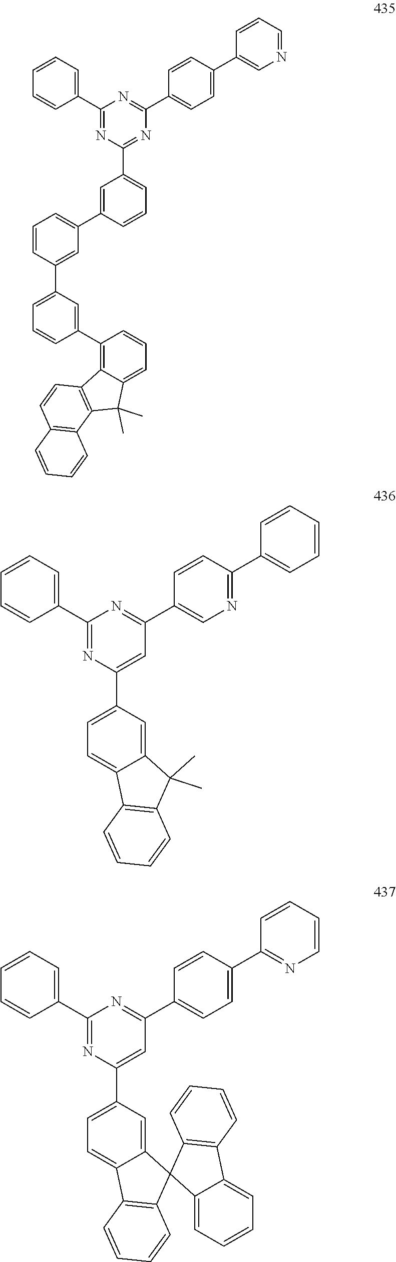 [SMILES: CC1(C)c2cccc(-c3cccc(-c4cccc(-c5cccc(-c6nc(-c7ccccc7)nc(-c7ccc(-c8cccnc8)cc7)n6)c5)c4)c3)c2-c2ccc3ccccc3c21 ; CC1(C)c2ccccc2-c2ccc(-c3cc(-c4ccc(-c5ccccc5)nc4)nc(-c4ccccc4)n3)cc21 ; c1ccc(-c2nc(-c3ccc(-c4ccccn4)cc3)cc(-c3ccc4c(c3)C3(c5ccccc5-c5ccccc53)c3ccccc3-4)n2)cc1]

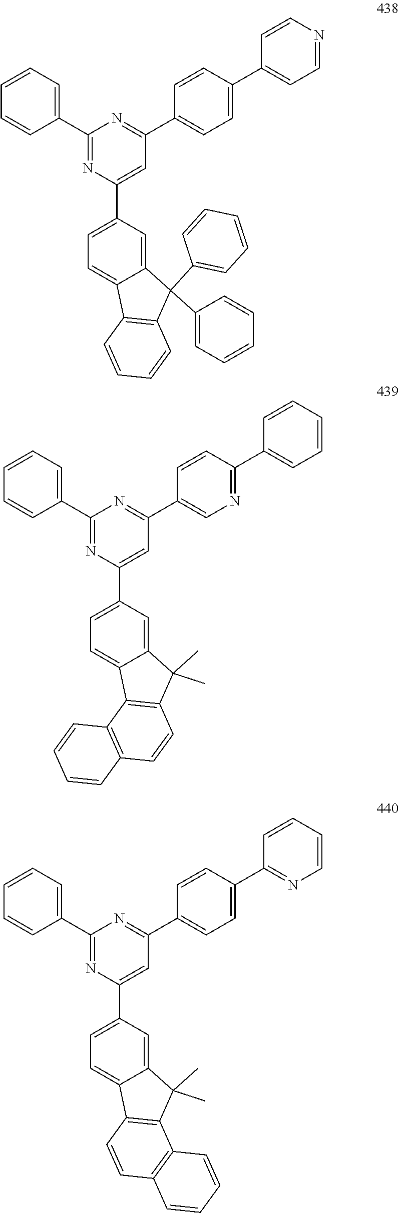 [SMILES: c1ccc(-c2nc(-c3ccc(-c4ccncc4)cc3)cc(-c3ccc4c(c3)C(c3ccccc3)(c3ccccc3)c3ccccc3-4)n2)cc1 ; CC1(C)c2cc(-c3cc(-c4ccc(-c5ccccc5)nc4)nc(-c4ccccc4)n3)ccc2-c2c1ccc1ccccc21 ; CC1(C)c2cc(-c3cc(-c4ccc(-c5ccccn5)cc4)nc(-c4ccccc4)n3)ccc2-c2ccc3ccccc3c21]

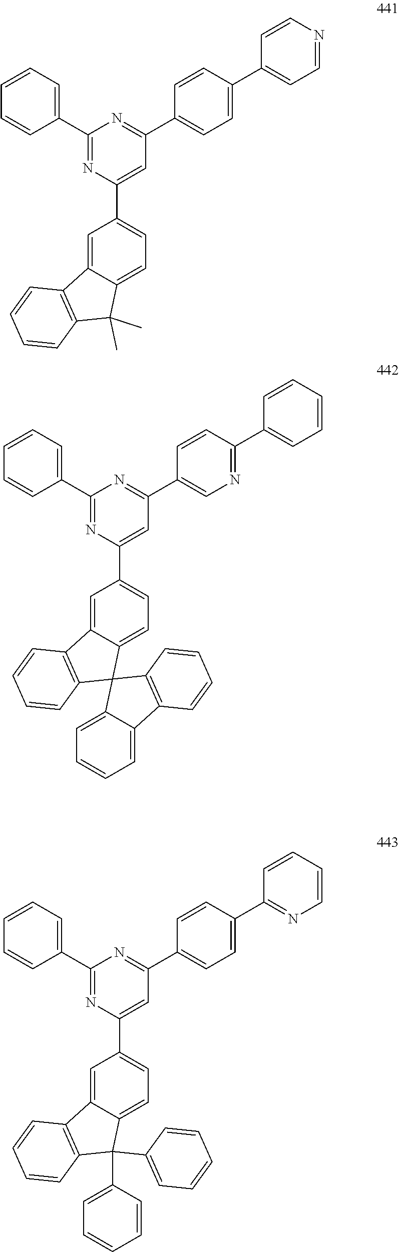 [SMILES: CC1(C)c2ccccc2-c2cc(-c3cc(-c4ccc(-c5ccncc5)cc4)nc(-c4ccccc4)n3)ccc21 ; c1ccc(-c2ccc(-c3cc(-c4ccc5c(c4)-c4ccccc4C54c5ccccc5-c5ccccc54)nc(-c4ccccc4)n3)cn2)cc1 ; c1ccc(-c2nc(-c3ccc(-c4ccccn4)cc3)cc(-c3ccc4c(c3)-c3ccccc3C4(c3ccccc3)c3ccccc3)n2)cc1]

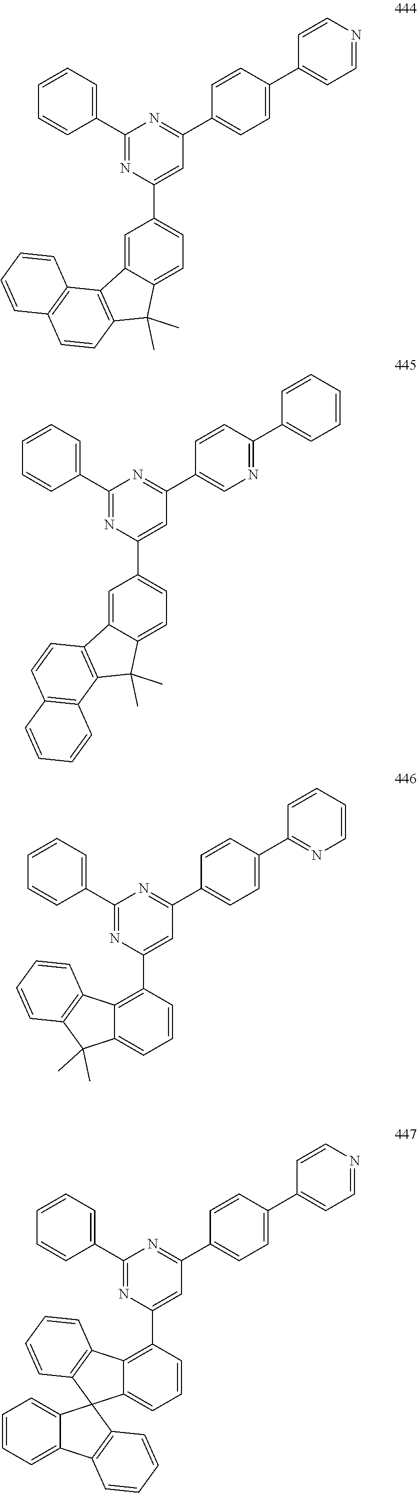 [SMILES: CC1(C)c2ccc(-c3cc(-c4ccc(-c5ccncc5)cc4)nc(-c4ccccc4)n3)cc2-c2c1ccc1ccccc21 ; CC1(C)c2ccc(-c3cc(-c4ccc(-c5ccccc5)nc4)nc(-c4ccccc4)n3)cc2-c2ccc3ccccc3c21 ; CC1(C)c2ccccc2-c2c(-c3cc(-c4ccc(-c5ccccn5)cc4)nc(-c4ccccc4)n3)cccc21 ; c1ccc(-c2nc(-c3ccc(-c4ccncc4)cc3)cc(-c3cccc4c3-c3ccccc3C43c4ccccc4-c4ccccc43)n2)cc1]

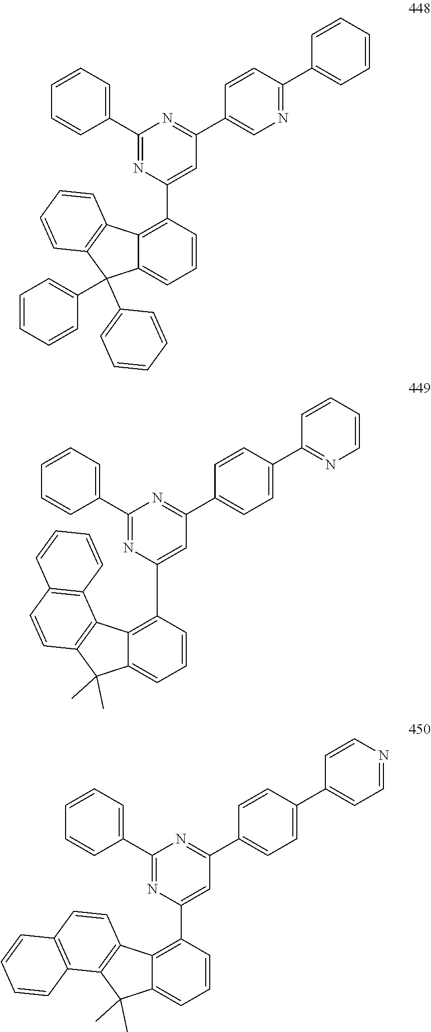 [SMILES: c1ccc(-c2ccc(-c3cc(-c4cccc5c4-c4ccccc4C5(c4ccccc4)c4ccccc4)nc(-c4ccccc4)n3)cn2)cc1 ; CC1(C)c2cccc(-c3cc(-c4ccc(-c5ccccn5)cc4)nc(-c4ccccc4)n3)c2-c2c1ccc1ccccc21 ; CC1(C)c2cccc(-c3cc(-c4ccc(-c5ccncc5)cc4)nc(-c4ccccc4)n3)c2-c2ccc3ccccc3c21]

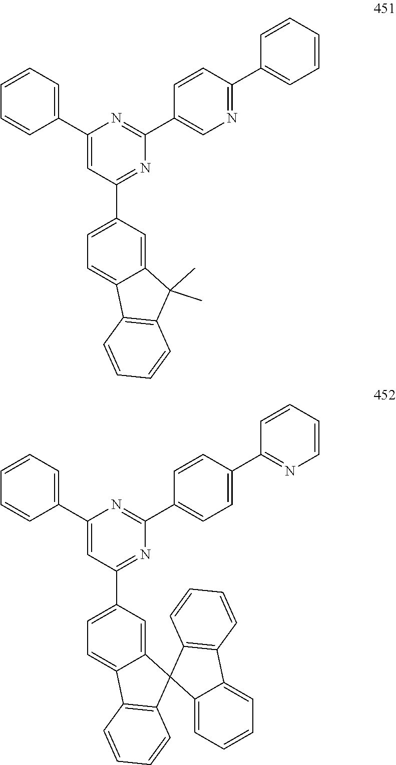 [SMILES: CC1(C)c2ccccc2-c2ccc(-c3cc(-c4ccccc4)nc(-c4ccc(-c5ccccc5)nc4)n3)cc21 ; c1ccc(-c2cc(-c3ccc4c(c3)C3(c5ccccc5-c5ccccc53)c3ccccc3-4)nc(-c3ccc(-c4ccccn4)cc3)n2)cc1]

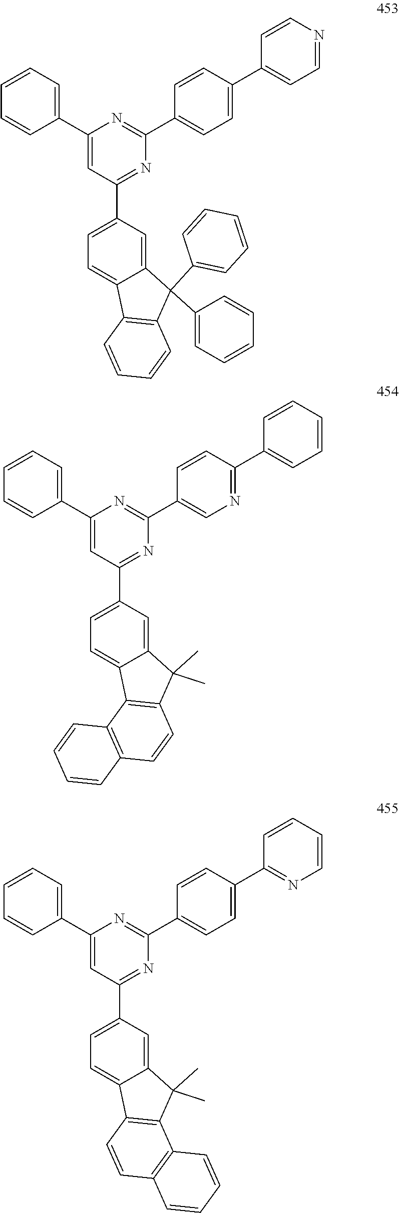 [SMILES: c1ccc(-c2cc(-c3ccc4c(c3)C(c3ccccc3)(c3ccccc3)c3ccccc3-4)nc(-c3ccc(-c4ccncc4)cc3)n2)cc1 ; CC1(C)c2cc(-c3cc(-c4ccccc4)nc(-c4ccc(-c5ccccc5)nc4)n3)ccc2-c2c1ccc1ccccc21 ; CC1(C)c2cc(-c3cc(-c4ccccc4)nc(-c4ccc(-c5ccccn5)cc4)n3)ccc2-c2ccc3ccccc3c21]

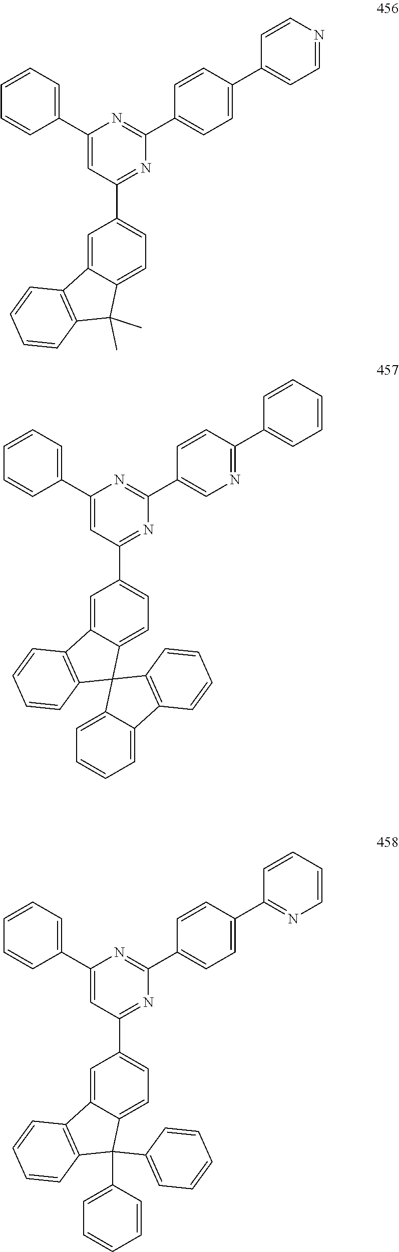 [SMILES: CC1(C)c2ccccc2-c2cc(-c3cc(-c4ccccc4)nc(-c4ccc(-c5ccncc5)cc4)n3)ccc21 ; c1ccc(-c2ccc(-c3nc(-c4ccccc4)cc(-c4ccc5c(c4)-c4ccccc4C54c5ccccc5-c5ccccc54)n3)cn2)cc1 ; c1ccc(-c2cc(-c3ccc4c(c3)-c3ccccc3C4(c3ccccc3)c3ccccc3)nc(-c3ccc(-c4ccccn4)cc3)n2)cc1]

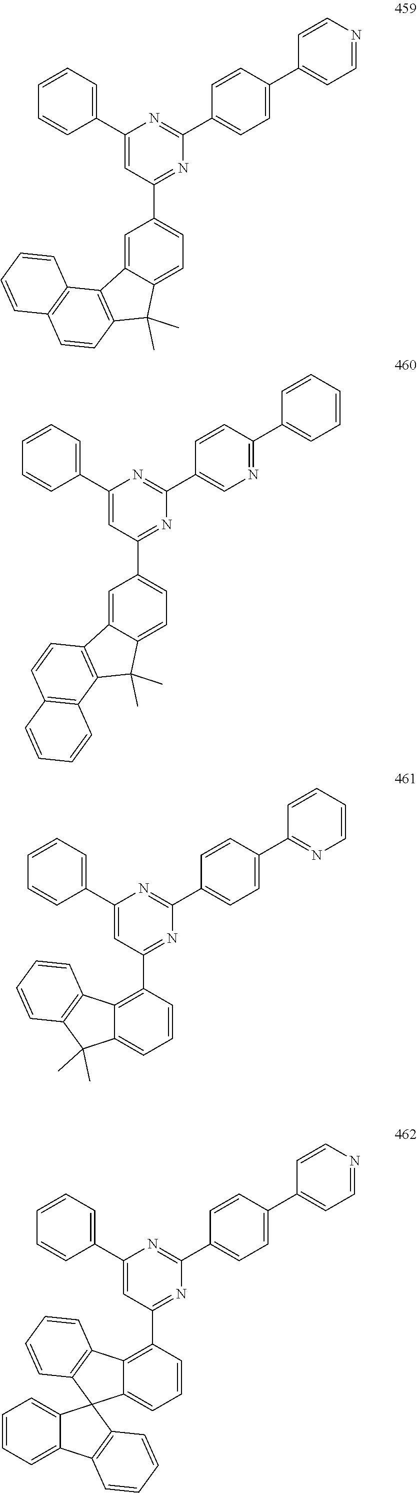 [SMILES: CC1(C)c2ccc(-c3cc(-c4ccccc4)nc(-c4ccc(-c5ccncc5)cc4)n3)cc2-c2c1ccc1ccccc21 ; CC1(C)c2ccc(-c3cc(-c4ccccc4)nc(-c4ccc(-c5ccccc5)nc4)n3)cc2-c2ccc3ccccc3c21 ; CC1(C)c2ccccc2-c2c(-c3cc(-c4ccccc4)nc(-c4ccc(-c5ccccn5)cc4)n3)cccc21 ; c1ccc(-c2cc(-c3cccc4c3-c3ccccc3C43c4ccccc4-c4ccccc43)nc(-c3ccc(-c4ccncc4)cc3)n2)cc1]

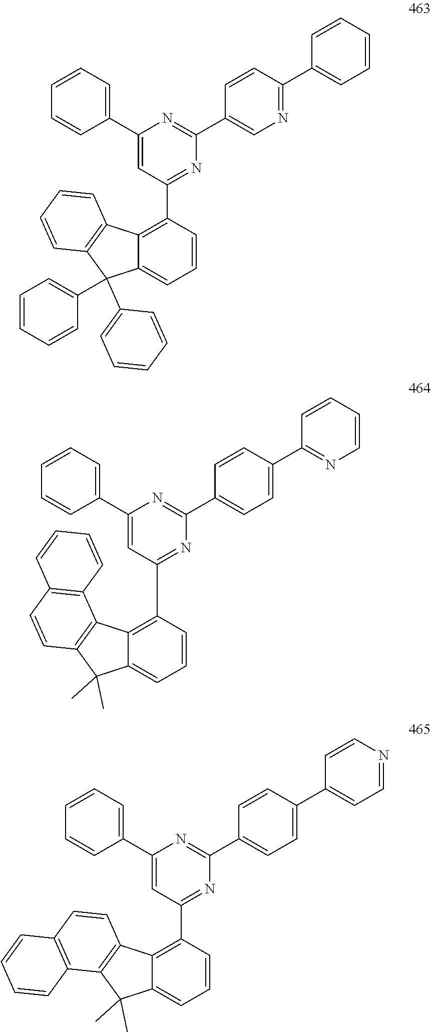 [SMILES: c1ccc(-c2ccc(-c3nc(-c4ccccc4)cc(-c4cccc5c4-c4ccccc4C5(c4ccccc4)c4ccccc4)n3)cn2)cc1 ; CC1(C)c2cccc(-c3cc(-c4ccccc4)nc(-c4ccc(-c5ccccn5)cc4)n3)c2-c2c1ccc1ccccc21 ; CC1(C)c2cccc(-c3cc(-c4ccccc4)nc(-c4ccc(-c5ccncc5)cc4)n3)c2-c2ccc3ccccc3c21]

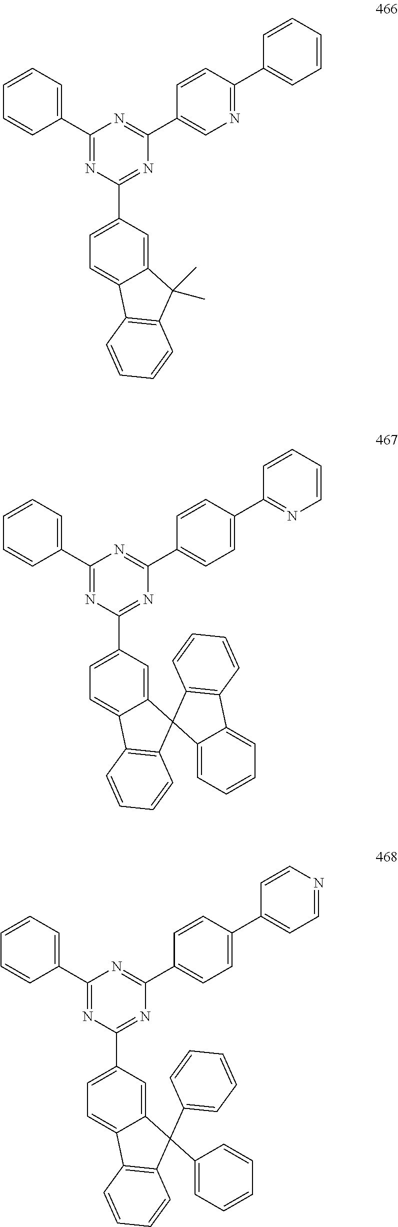 [SMILES: CC1(C)c2ccccc2-c2ccc(-c3nc(-c4ccccc4)nc(-c4ccc(-c5ccccc5)nc4)n3)cc21 ; c1ccc(-c2nc(-c3ccc(-c4ccccn4)cc3)nc(-c3ccc4c(c3)C3(c5ccccc5-c5ccccc53)c3ccccc3-4)n2)cc1 ; c1ccc(-c2nc(-c3ccc(-c4ccncc4)cc3)nc(-c3ccc4c(c3)C(c3ccccc3)(c3ccccc3)c3ccccc3-4)n2)cc1]

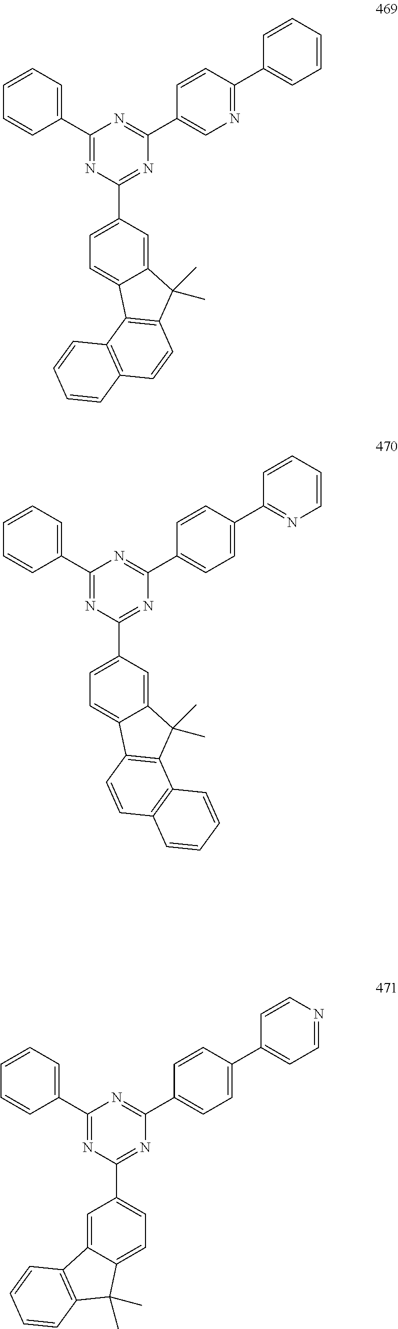 [SMILES: CC1(C)c2cc(-c3nc(-c4ccccc4)nc(-c4ccc(-c5ccccc5)nc4)n3)ccc2-c2c1ccc1ccccc21 ; CC1(C)c2cc(-c3nc(-c4ccccc4)nc(-c4ccc(-c5ccccn5)cc4)n3)ccc2-c2ccc3ccccc3c21 ; CC1(C)c2ccccc2-c2cc(-c3nc(-c4ccccc4)nc(-c4ccc(-c5ccncc5)cc4)n3)ccc21]

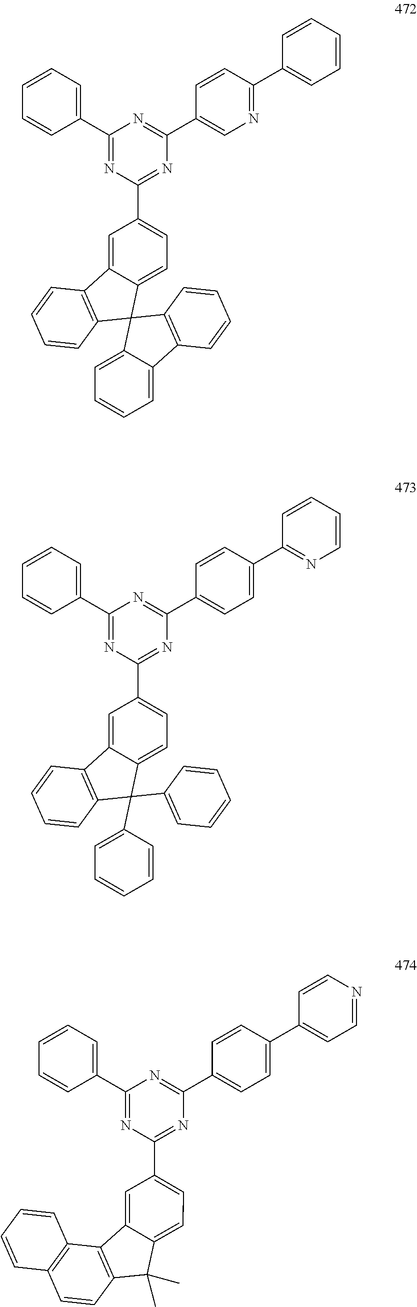 [SMILES: c1ccc(-c2ccc(-c3nc(-c4ccccc4)nc(-c4ccc5c(c4)-c4ccccc4C54c5ccccc5-c5ccccc54)n3)cn2)cc1 ; c1ccc(-c2nc(-c3ccc(-c4ccccn4)cc3)nc(-c3ccc4c(c3)-c3ccccc3C4(c3ccccc3)c3ccccc3)n2)cc1 ; CC1(C)c2ccc(-c3nc(-c4ccccc4)nc(-c4ccc(-c5ccncc5)cc4)n3)cc2-c2c1ccc1ccccc21]

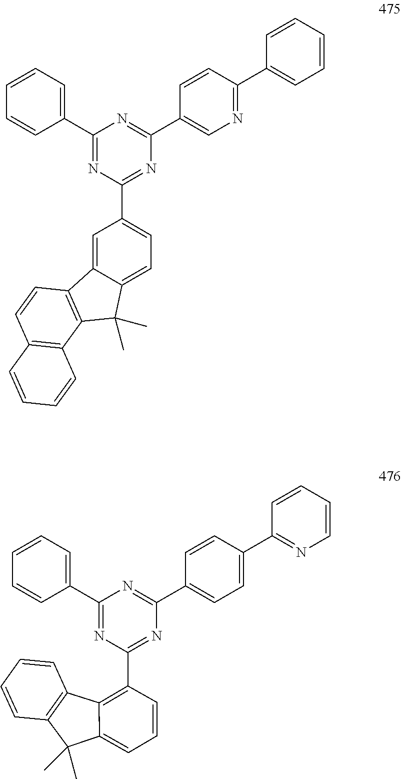 [SMILES: CC1(C)c2ccc(-c3nc(-c4ccccc4)nc(-c4ccc(-c5ccccc5)nc4)n3)cc2-c2ccc3ccccc3c21 ; CC1(C)c2ccccc2-c2c(-c3nc(-c4ccccc4)nc(-c4ccc(-c5ccccn5)cc4)n3)cccc21]

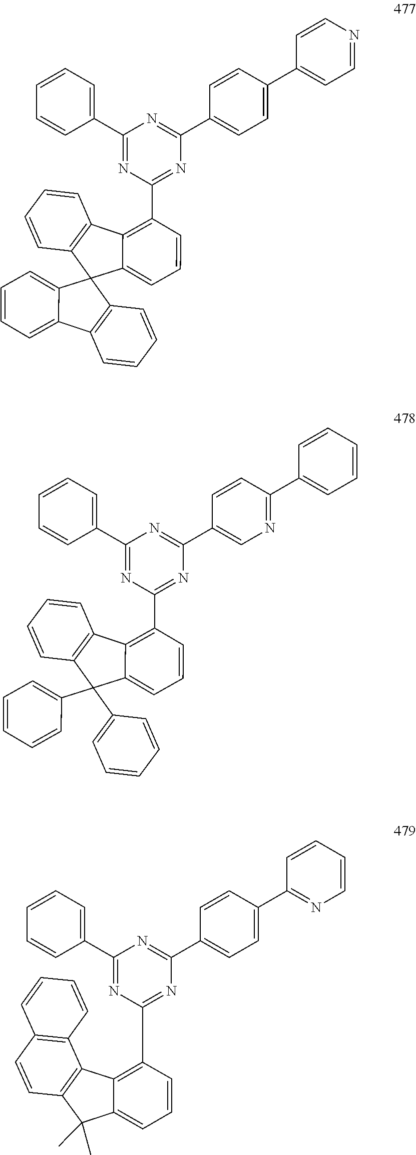 [SMILES: c1ccc(-c2nc(-c3ccc(-c4ccncc4)cc3)nc(-c3cccc4c3-c3ccccc3C43c4ccccc4-c4ccccc43)n2)cc1 ; c1ccc(-c2ccc(-c3nc(-c4ccccc4)nc(-c4cccc5c4-c4ccccc4C5(c4ccccc4)c4ccccc4)n3)cn2)cc1 ; CC1(C)c2cccc(-c3nc(-c4ccccc4)nc(-c4ccc(-c5ccccn5)cc4)n3)c2-c2c1ccc1ccccc21]

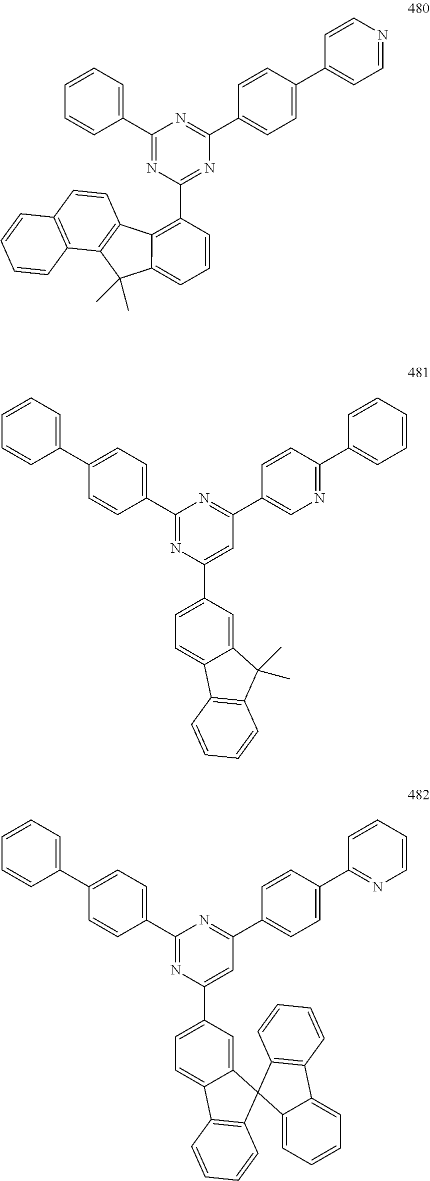 [SMILES: CC1(C)c2cccc(-c3nc(-c4ccccc4)nc(-c4ccc(-c5ccncc5)cc4)n3)c2-c2ccc3ccccc3c21 ; CC1(C)c2ccccc2-c2ccc(-c3cc(-c4ccc(-c5ccccc5)nc4)nc(-c4ccc(-c5ccccc5)cc4)n3)cc21 ; c1ccc(-c2ccc(-c3nc(-c4ccc(-c5ccccn5)cc4)cc(-c4ccc5c(c4)C4(c6ccccc6-c6ccccc64)c4ccccc4-5)n3)cc2)cc1]

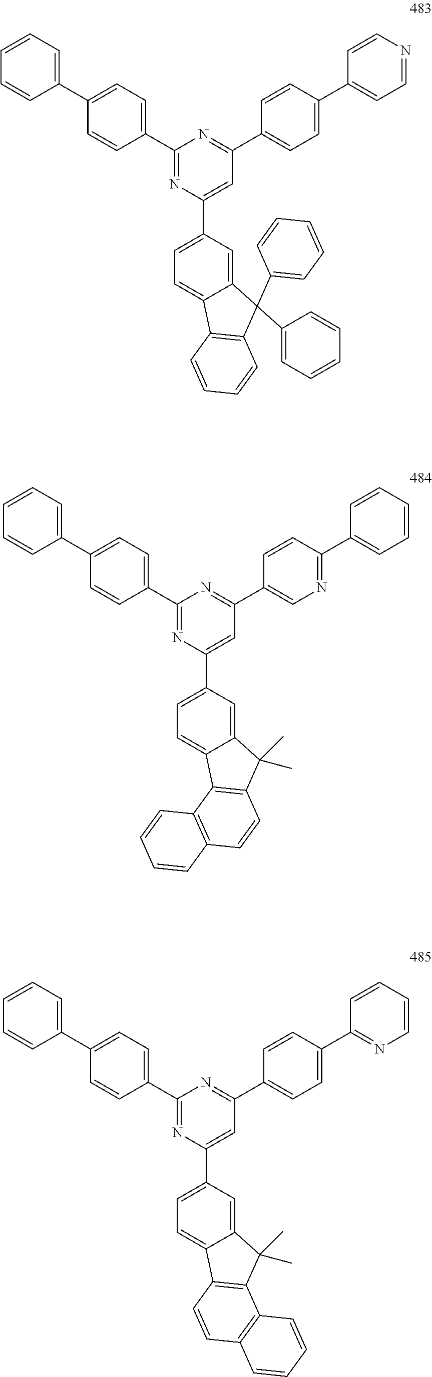 [SMILES: c1ccc(-c2ccc(-c3nc(-c4ccc(-c5ccncc5)cc4)cc(-c4ccc5c(c4)C(c4ccccc4)(c4ccccc4)c4ccccc4-5)n3)cc2)cc1 ; CC1(C)c2cc(-c3cc(-c4ccc(-c5ccccc5)nc4)nc(-c4ccc(-c5ccccc5)cc4)n3)ccc2-c2c1ccc1ccccc21 ; CC1(C)c2cc(-c3cc(-c4ccc(-c5ccccn5)cc4)nc(-c4ccc(-c5ccccc5)cc4)n3)ccc2-c2ccc3ccccc3c21]

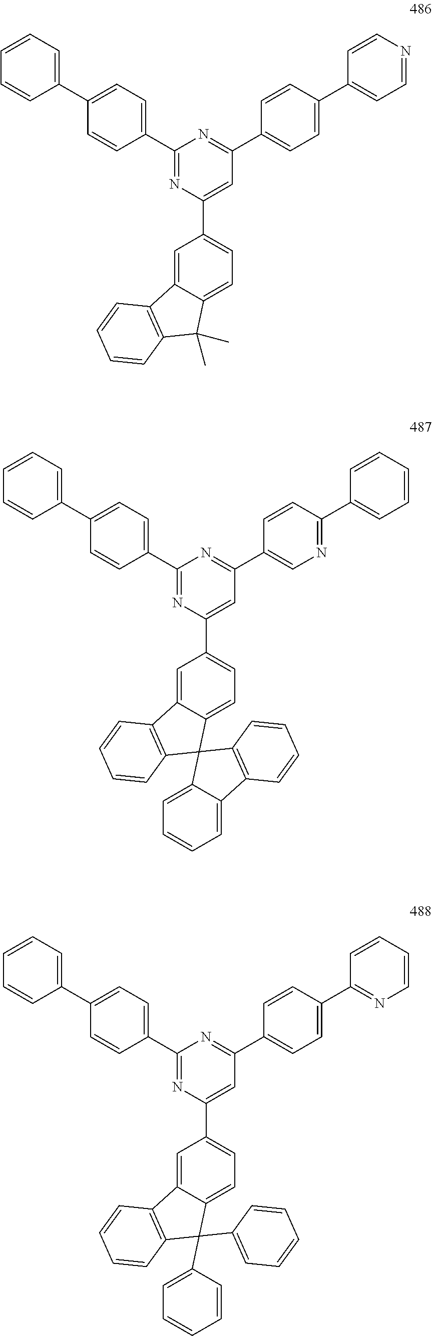 [SMILES: CC1(C)c2ccccc2-c2cc(-c3cc(-c4ccc(-c5ccncc5)cc4)nc(-c4ccc(-c5ccccc5)cc4)n3)ccc21 ; c1ccc(-c2ccc(-c3nc(-c4ccc(-c5ccccc5)nc4)cc(-c4ccc5c(c4)-c4ccccc4C54c5ccccc5-c5ccccc54)n3)cc2)cc1 ; c1ccc(-c2ccc(-c3nc(-c4ccc(-c5ccccn5)cc4)cc(-c4ccc5c(c4)-c4ccccc4C5(c4ccccc4)c4ccccc4)n3)cc2)cc1]

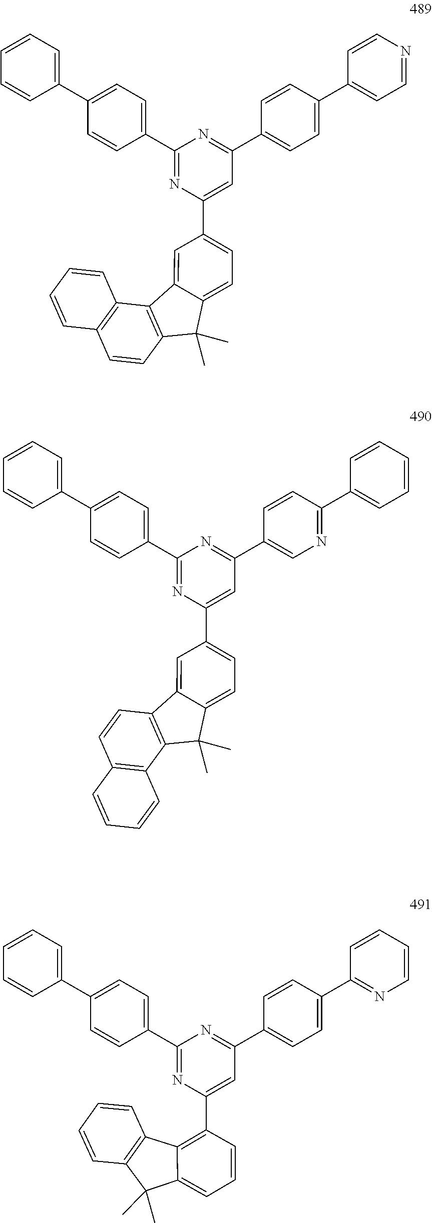 [SMILES: CC1(C)c2ccc(-c3cc(-c4ccc(-c5ccncc5)cc4)nc(-c4ccc(-c5ccccc5)cc4)n3)cc2-c2c1ccc1ccccc21 ; CC1(C)c2ccc(-c3cc(-c4ccc(-c5ccccc5)nc4)nc(-c4ccc(-c5ccccc5)cc4)n3)cc2-c2ccc3ccccc3c21 ; CC1(C)c2ccccc2-c2c(-c3cc(-c4ccc(-c5ccccn5)cc4)nc(-c4ccc(-c5ccccc5)cc4)n3)cccc21]

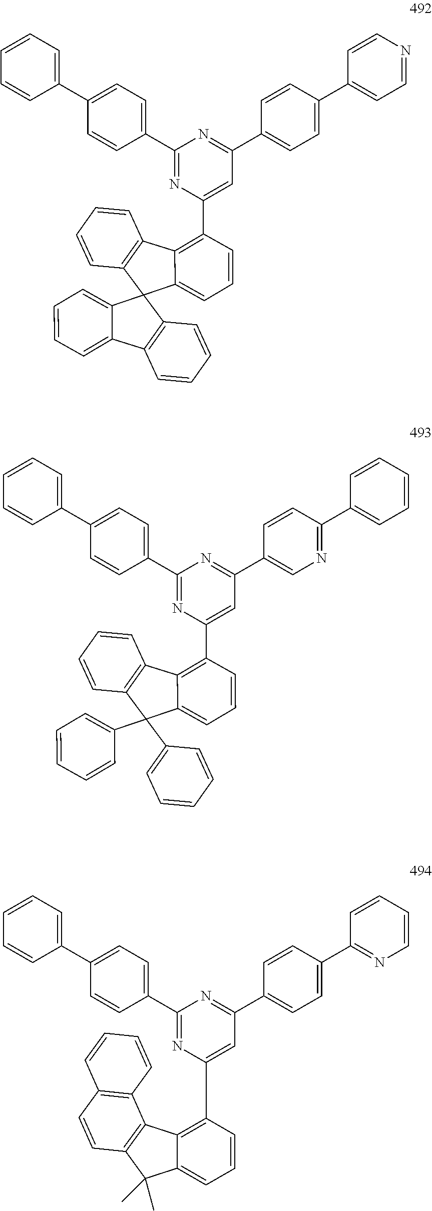 [SMILES: c1ccc(-c2ccc(-c3nc(-c4ccc(-c5ccncc5)cc4)cc(-c4cccc5c4-c4ccccc4C54c5ccccc5-c5ccccc54)n3)cc2)cc1 ; c1ccc(-c2ccc(-c3nc(-c4ccc(-c5ccccc5)nc4)cc(-c4cccc5c4-c4ccccc4C5(c4ccccc4)c4ccccc4)n3)cc2)cc1 ; CC1(C)c2cccc(-c3cc(-c4ccc(-c5ccccn5)cc4)nc(-c4ccc(-c5ccccc5)cc4)n3)c2-c2c1ccc1ccccc21]

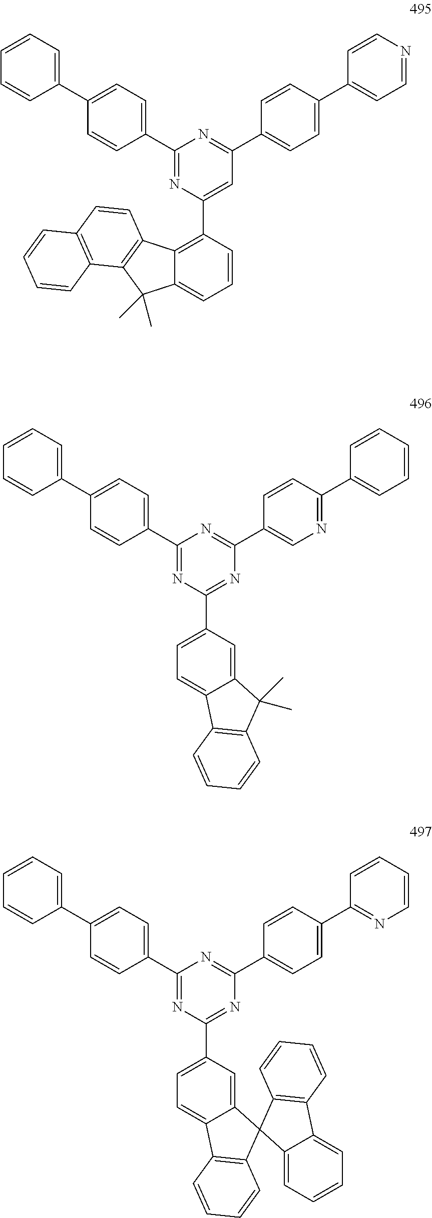 [SMILES: CC1(C)c2cccc(-c3cc(-c4ccc(-c5ccncc5)cc4)nc(-c4ccc(-c5ccccc5)cc4)n3)c2-c2ccc3ccccc3c21 ; CC1(C)c2ccccc2-c2ccc(-c3nc(-c4ccc(-c5ccccc5)cc4)nc(-c4ccc(-c5ccccc5)nc4)n3)cc21 ; c1ccc(-c2ccc(-c3nc(-c4ccc(-c5ccccn5)cc4)nc(-c4ccc5c(c4)C4(c6ccccc6-c6ccccc64)c4ccccc4-5)n3)cc2)cc1]

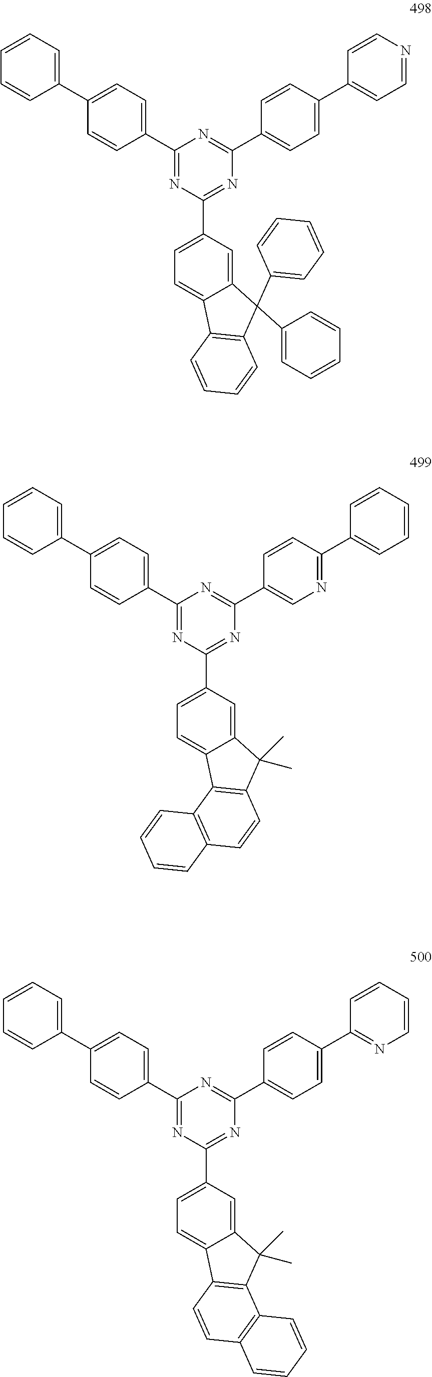 [SMILES: c1ccc(-c2ccc(-c3nc(-c4ccc(-c5ccncc5)cc4)nc(-c4ccc5c(c4)C(c4ccccc4)(c4ccccc4)c4ccccc4-5)n3)cc2)cc1 ; CC1(C)c2cc(-c3nc(-c4ccc(-c5ccccc5)cc4)nc(-c4ccc(-c5ccccc5)nc4)n3)ccc2-c2c1ccc1ccccc21 ; CC1(C)c2cc(-c3nc(-c4ccc(-c5ccccc5)cc4)nc(-c4ccc(-c5ccccn5)cc4)n3)ccc2-c2ccc3ccccc3c21]

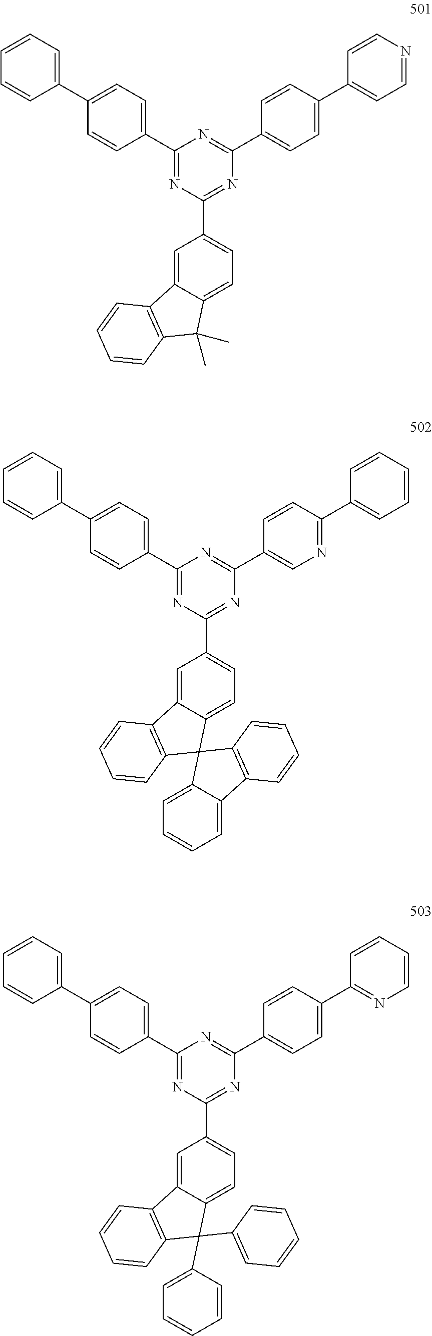 [SMILES: CC1(C)c2ccccc2-c2cc(-c3nc(-c4ccc(-c5ccccc5)cc4)nc(-c4ccc(-c5ccncc5)cc4)n3)ccc21 ; c1ccc(-c2ccc(-c3nc(-c4ccc(-c5ccccc5)nc4)nc(-c4ccc5c(c4)-c4ccccc4C54c5ccccc5-c5ccccc54)n3)cc2)cc1 ; c1ccc(-c2ccc(-c3nc(-c4ccc(-c5ccccn5)cc4)nc(-c4ccc5c(c4)-c4ccccc4C5(c4ccccc4)c4ccccc4)n3)cc2)cc1]

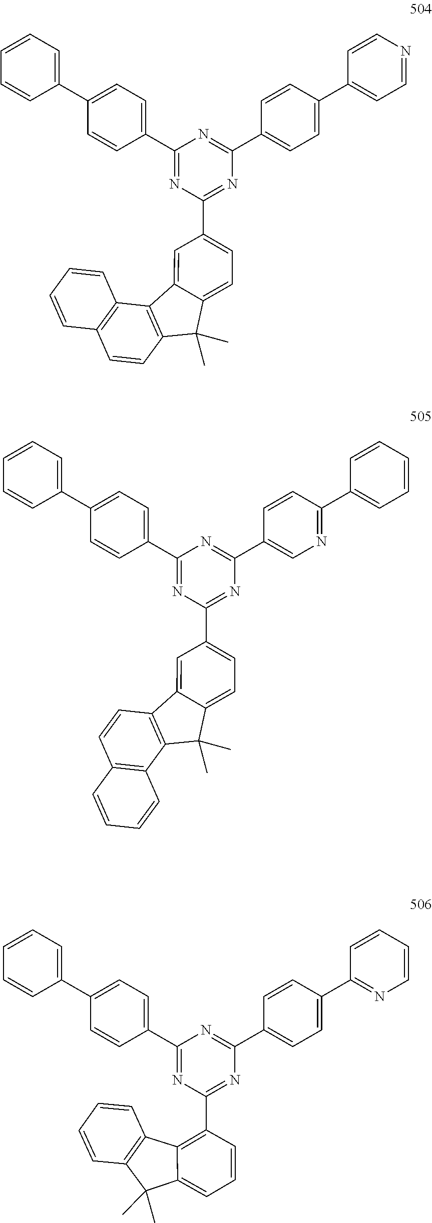 [SMILES: CC1(C)c2ccc(-c3nc(-c4ccc(-c5ccccc5)cc4)nc(-c4ccc(-c5ccncc5)cc4)n3)cc2-c2c1ccc1ccccc21 ; CC1(C)c2ccc(-c3nc(-c4ccc(-c5ccccc5)cc4)nc(-c4ccc(-c5ccccc5)nc4)n3)cc2-c2ccc3ccccc3c21 ; CC1(C)c2ccccc2-c2c(-c3nc(-c4ccc(-c5ccccc5)cc4)nc(-c4ccc(-c5ccccn5)cc4)n3)cccc21]

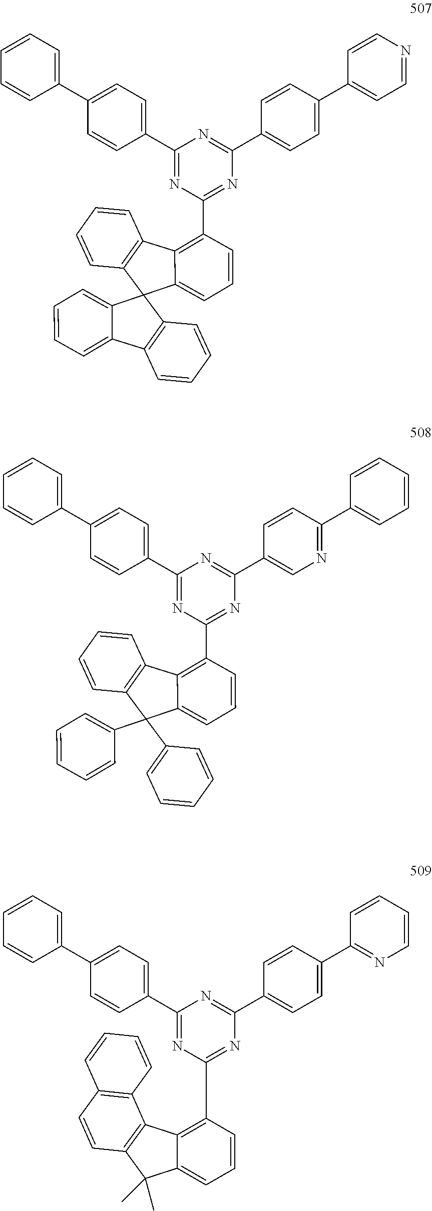 [SMILES: c1ccc(-c2ccc(-c3nc(-c4ccc(-c5ccncc5)cc4)nc(-c4cccc5c4-c4ccccc4C54c5ccccc5-c5ccccc54)n3)cc2)cc1 ; c1ccc(-c2ccc(-c3nc(-c4ccc(-c5ccccc5)nc4)nc(-c4cccc5c4-c4ccccc4C5(c4ccccc4)c4ccccc4)n3)cc2)cc1 ; CC1(C)c2cccc(-c3nc(-c4ccc(-c5ccccc5)cc4)nc(-c4ccc(-c5ccccn5)cc4)n3)c2-c2c1ccc1ccccc21]

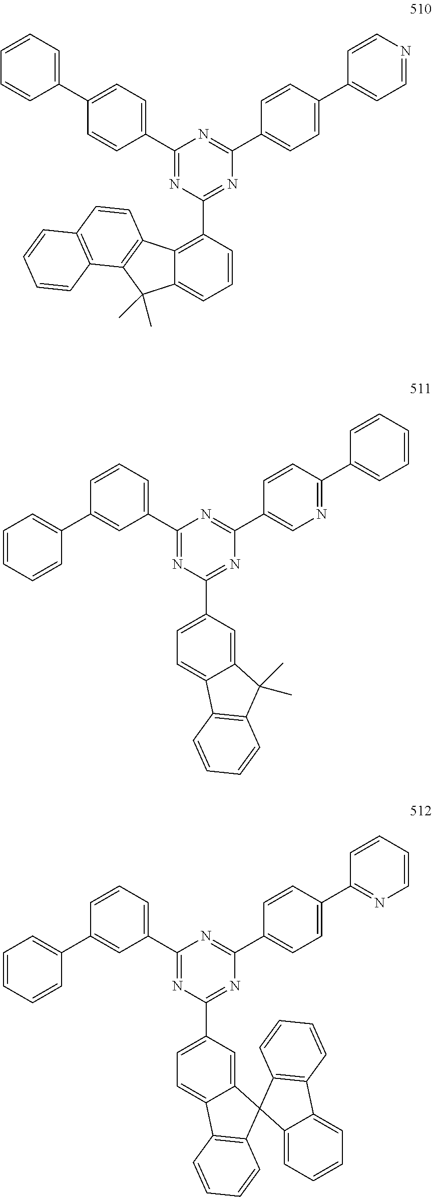 [SMILES: CC1(C)c2cccc(-c3nc(-c4ccc(-c5ccccc5)cc4)nc(-c4ccc(-c5ccncc5)cc4)n3)c2-c2ccc3ccccc3c21 ; CC1(C)c2ccccc2-c2ccc(-c3nc(-c4ccc(-c5ccccc5)nc4)nc(-c4cccc(-c5ccccc5)c4)n3)cc21 ; c1ccc(-c2cccc(-c3nc(-c4ccc(-c5ccccn5)cc4)nc(-c4ccc5c(c4)C4(c6ccccc6-c6ccccc64)c4ccccc4-5)n3)c2)cc1]

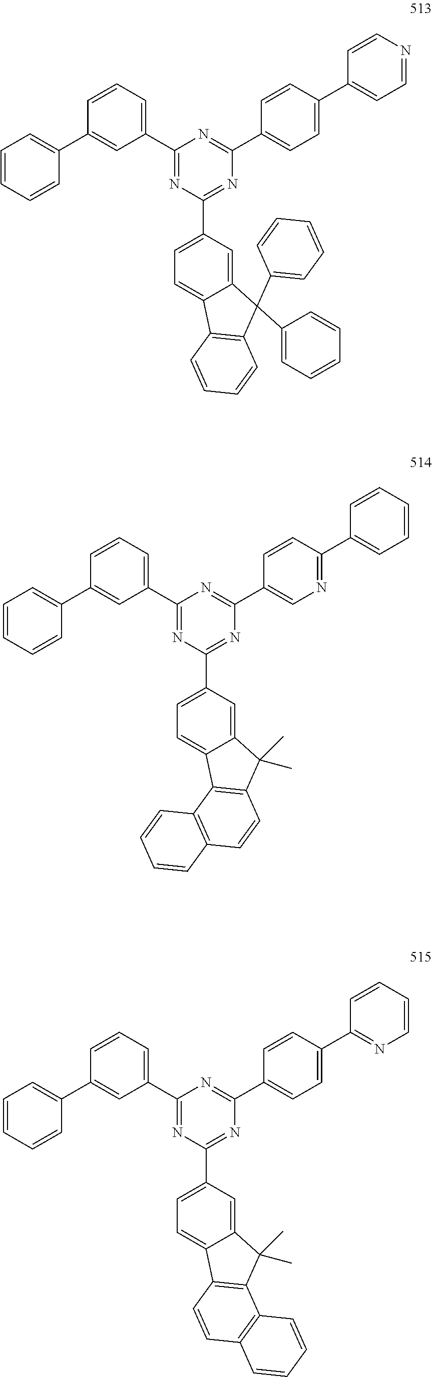 [SMILES: c1ccc(-c2cccc(-c3nc(-c4ccc(-c5ccncc5)cc4)nc(-c4ccc5c(c4)C(c4ccccc4)(c4ccccc4)c4ccccc4-5)n3)c2)cc1 ; CC1(C)c2cc(-c3nc(-c4ccc(-c5ccccc5)nc4)nc(-c4cccc(-c5ccccc5)c4)n3)ccc2-c2c1ccc1ccccc21 ; CC1(C)c2cc(-c3nc(-c4ccc(-c5ccccn5)cc4)nc(-c4cccc(-c5ccccc5)c4)n3)ccc2-c2ccc3ccccc3c21]

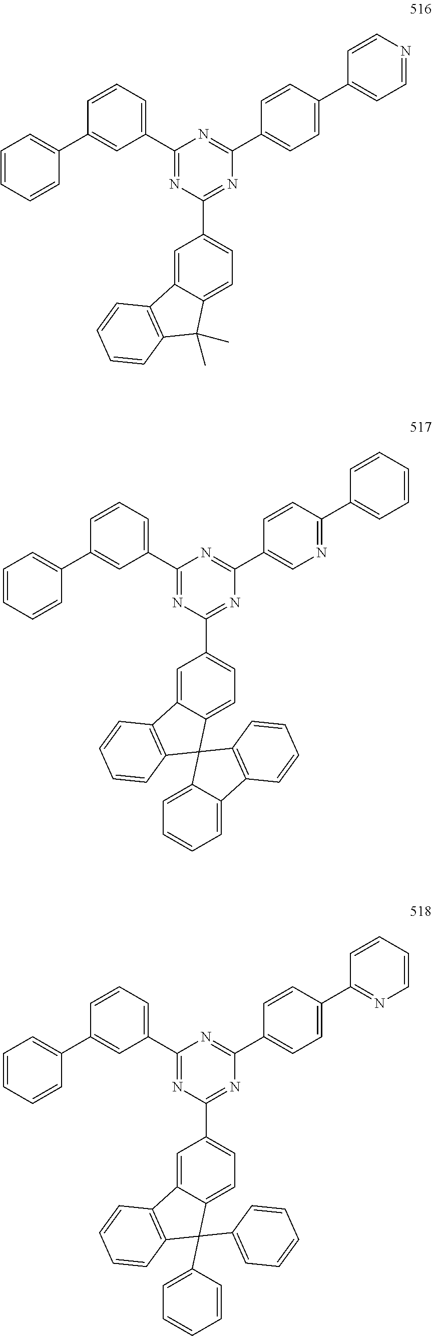 [SMILES: CC1(C)c2ccccc2-c2cc(-c3nc(-c4ccc(-c5ccncc5)cc4)nc(-c4cccc(-c5ccccc5)c4)n3)ccc21 ; c1ccc(-c2cccc(-c3nc(-c4ccc(-c5ccccc5)nc4)nc(-c4ccc5c(c4)-c4ccccc4C54c5ccccc5-c5ccccc54)n3)c2)cc1 ; c1ccc(-c2cccc(-c3nc(-c4ccc(-c5ccccn5)cc4)nc(-c4ccc5c(c4)-c4ccccc4C5(c4ccccc4)c4ccccc4)n3)c2)cc1]

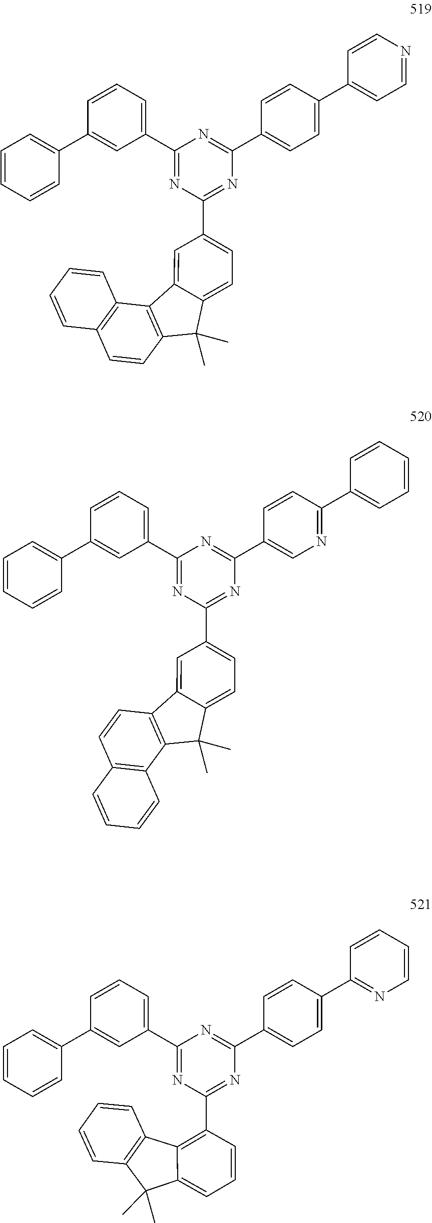 [SMILES: CC1(C)c2ccc(-c3nc(-c4ccc(-c5ccncc5)cc4)nc(-c4cccc(-c5ccccc5)c4)n3)cc2-c2c1ccc1ccccc21 ; CC1(C)c2ccc(-c3nc(-c4ccc(-c5ccccc5)nc4)nc(-c4cccc(-c5ccccc5)c4)n3)cc2-c2ccc3ccccc3c21 ; CC1(C)c2ccccc2-c2c(-c3nc(-c4ccc(-c5ccccn5)cc4)nc(-c4cccc(-c5ccccc5)c4)n3)cccc21]

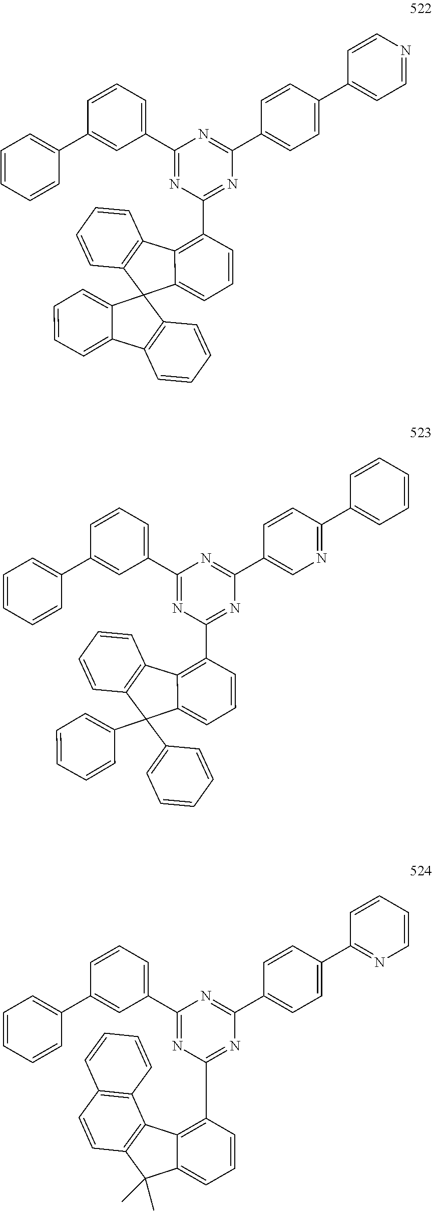 [SMILES: c1ccc(-c2cccc(-c3nc(-c4ccc(-c5ccncc5)cc4)nc(-c4cccc5c4-c4ccccc4C54c5ccccc5-c5ccccc54)n3)c2)cc1 ; c1ccc(-c2cccc(-c3nc(-c4ccc(-c5ccccc5)nc4)nc(-c4cccc5c4-c4ccccc4C5(c4ccccc4)c4ccccc4)n3)c2)cc1 ; CC1(C)c2cccc(-c3nc(-c4ccc(-c5ccccn5)cc4)nc(-c4cccc(-c5ccccc5)c4)n3)c2-c2c1ccc1ccccc21]

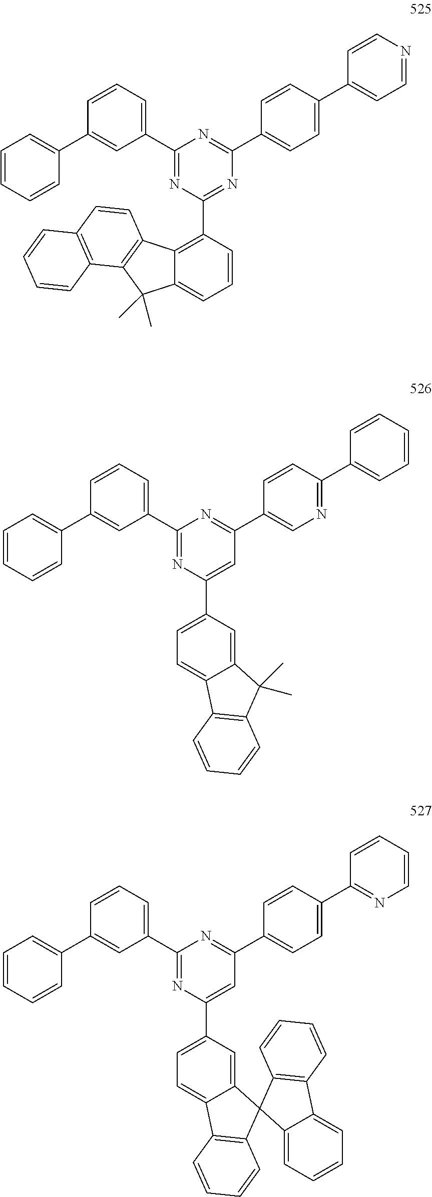 [SMILES: CC1(C)c2cccc(-c3nc(-c4ccc(-c5ccncc5)cc4)nc(-c4cccc(-c5ccccc5)c4)n3)c2-c2ccc3ccccc3c21 ; CC1(C)c2ccccc2-c2ccc(-c3cc(-c4ccc(-c5ccccc5)nc4)nc(-c4cccc(-c5ccccc5)c4)n3)cc21 ; c1ccc(-c2cccc(-c3nc(-c4ccc(-c5ccccn5)cc4)cc(-c4ccc5c(c4)C4(c6ccccc6-c6ccccc64)c4ccccc4-5)n3)c2)cc1]

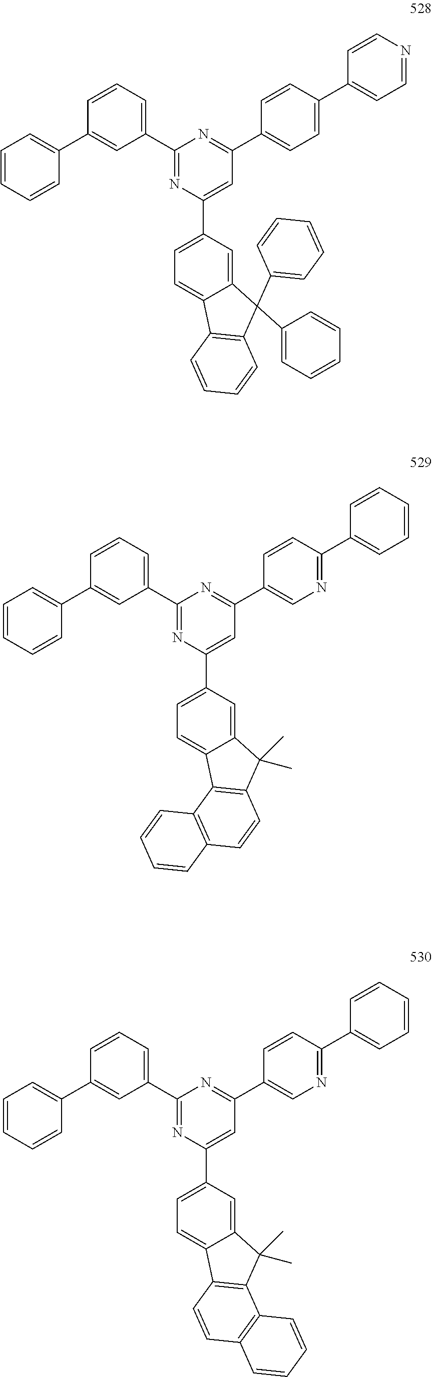 [SMILES: c1ccc(-c2cccc(-c3nc(-c4ccc(-c5ccncc5)cc4)cc(-c4ccc5c(c4)C(c4ccccc4)(c4ccccc4)c4ccccc4-5)n3)c2)cc1 ; CC1(C)c2cc(-c3cc(-c4ccc(-c5ccccc5)nc4)nc(-c4cccc(-c5ccccc5)c4)n3)ccc2-c2c1ccc1ccccc21 ; CC1(C)c2cc(-c3cc(-c4ccc(-c5ccccc5)nc4)nc(-c4cccc(-c5ccccc5)c4)n3)ccc2-c2ccc3ccccc3c21]

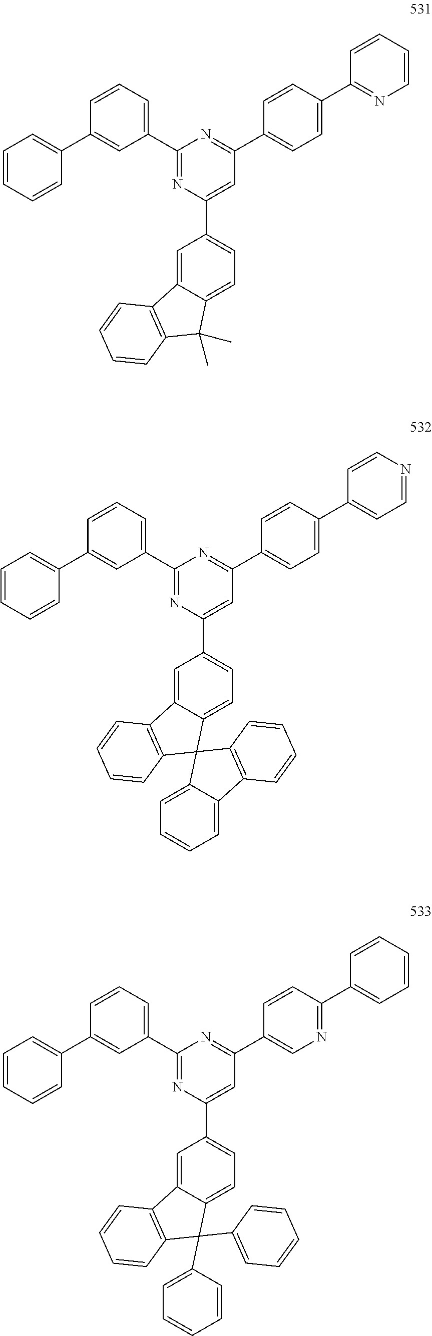 [SMILES: CC1(C)c2ccccc2-c2cc(-c3cc(-c4ccc(-c5ccccn5)cc4)nc(-c4cccc(-c5ccccc5)c4)n3)ccc21 ; c1ccc(-c2cccc(-c3nc(-c4ccc(-c5ccncc5)cc4)cc(-c4ccc5c(c4)-c4ccccc4C54c5ccccc5-c5ccccc54)n3)c2)cc1 ; c1ccc(-c2cccc(-c3nc(-c4ccc(-c5ccccc5)nc4)cc(-c4ccc5c(c4)-c4ccccc4C5(c4ccccc4)c4ccccc4)n3)c2)cc1]

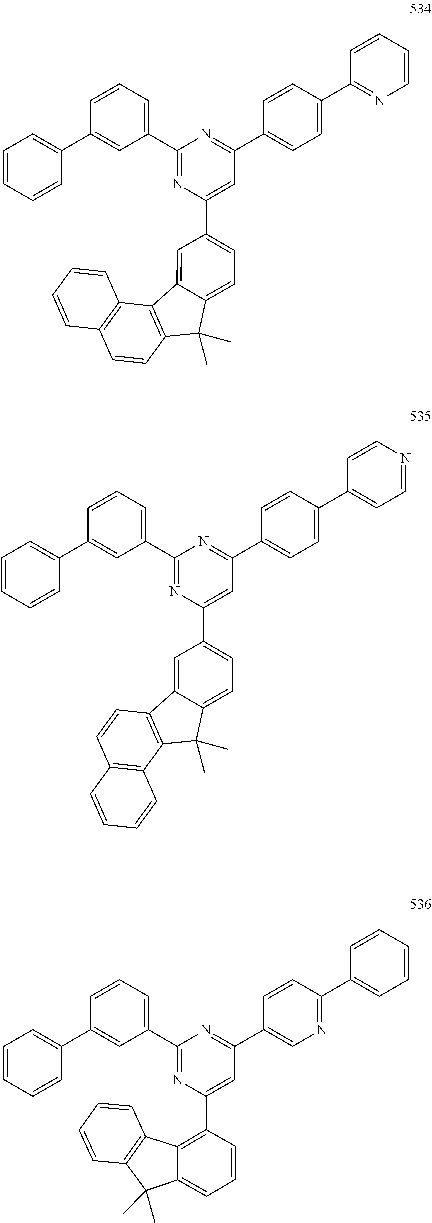 [SMILES: CC1(C)c2ccc(-c3cc(-c4ccc(-c5ccccn5)cc4)nc(-c4cccc(-c5ccccc5)c4)n3)cc2-c2c1ccc1ccccc21 ; CC1(C)c2ccc(-c3cc(-c4ccc(-c5ccncc5)cc4)nc(-c4cccc(-c5ccccc5)c4)n3)cc2-c2ccc3ccccc3c21 ; CC1(C)c2ccccc2-c2c(-c3cc(-c4ccc(-c5ccccc5)nc4)nc(-c4cccc(-c5ccccc5)c4)n3)cccc21]

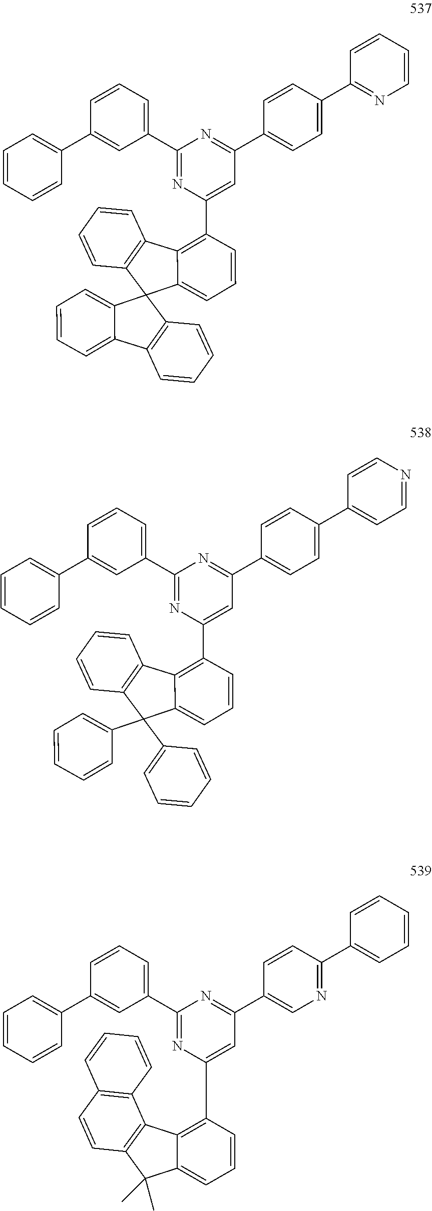 [SMILES: c1ccc(-c2cccc(-c3nc(-c4ccc(-c5ccccn5)cc4)cc(-c4cccc5c4-c4ccccc4C54c5ccccc5-c5ccccc54)n3)c2)cc1 ; c1ccc(-c2cccc(-c3nc(-c4ccc(-c5ccncc5)cc4)cc(-c4cccc5c4-c4ccccc4C5(c4ccccc4)c4ccccc4)n3)c2)cc1 ; CC1(C)c2cccc(-c3cc(-c4ccc(-c5ccccc5)nc4)nc(-c4cccc(-c5ccccc5)c4)n3)c2-c2c1ccc1ccccc21]

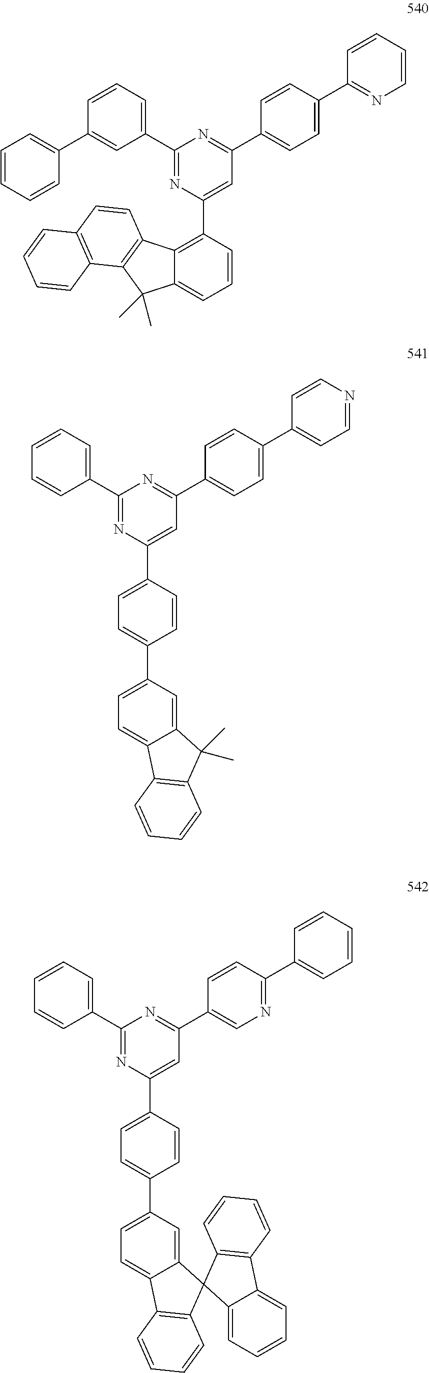 [SMILES: CC1(C)c2cccc(-c3cc(-c4ccc(-c5ccccn5)cc4)nc(-c4cccc(-c5ccccc5)c4)n3)c2-c2ccc3ccccc3c21 ; CC1(C)c2ccccc2-c2ccc(-c3ccc(-c4cc(-c5ccc(-c6ccncc6)cc5)nc(-c5ccccc5)n4)cc3)cc21 ; c1ccc(-c2ccc(-c3cc(-c4ccc(-c5ccc6c(c5)C5(c7ccccc7-c7ccccc75)c5ccccc5-6)cc4)nc(-c4ccccc4)n3)cn2)cc1]

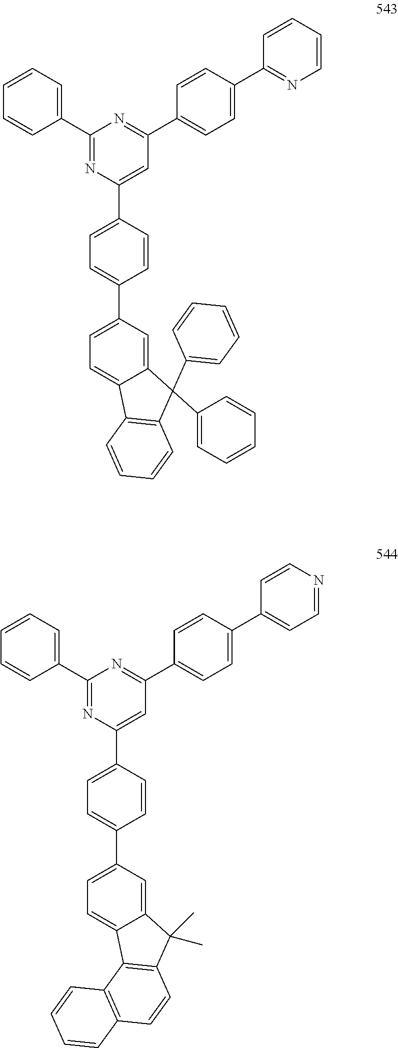 [SMILES: c1ccc(-c2nc(-c3ccc(-c4ccc5c(c4)C(c4ccccc4)(c4ccccc4)c4ccccc4-5)cc3)cc(-c3ccc(-c4ccccn4)cc3)n2)cc1 ; CC1(C)c2cc(-c3ccc(-c4cc(-c5ccc(-c6ccncc6)cc5)nc(-c5ccccc5)n4)cc3)ccc2-c2c1ccc1ccccc21]

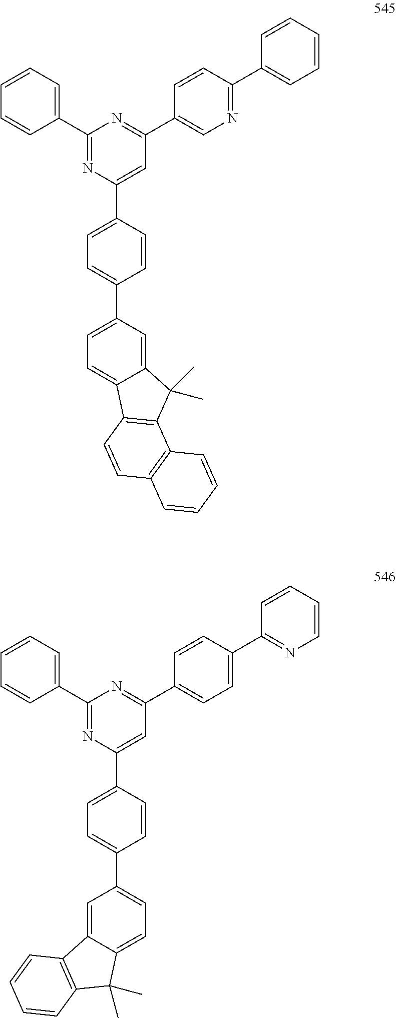 [SMILES: CC1(C)c2cc(-c3ccc(-c4cc(-c5ccc(-c6ccccc6)nc5)nc(-c5ccccc5)n4)cc3)ccc2-c2ccc3ccccc3c21 ; CC1(C)c2ccccc2-c2cc(-c3ccc(-c4cc(-c5ccc(-c6ccccn6)cc5)nc(-c5ccccc5)n4)cc3)ccc21]

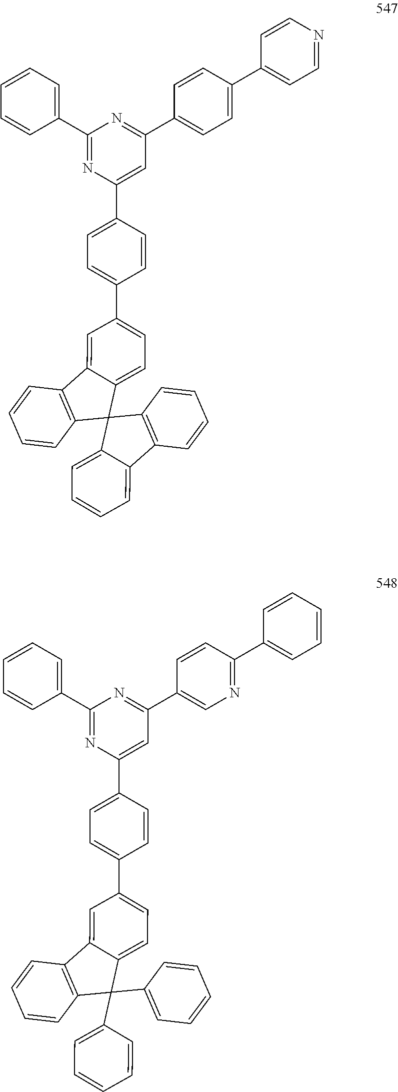 [SMILES: c1ccc(-c2nc(-c3ccc(-c4ccncc4)cc3)cc(-c3ccc(-c4ccc5c(c4)-c4ccccc4C54c5ccccc5-c5ccccc54)cc3)n2)cc1 ; c1ccc(-c2ccc(-c3cc(-c4ccc(-c5ccc6c(c5)-c5ccccc5C6(c5ccccc5)c5ccccc5)cc4)nc(-c4ccccc4)n3)cn2)cc1]

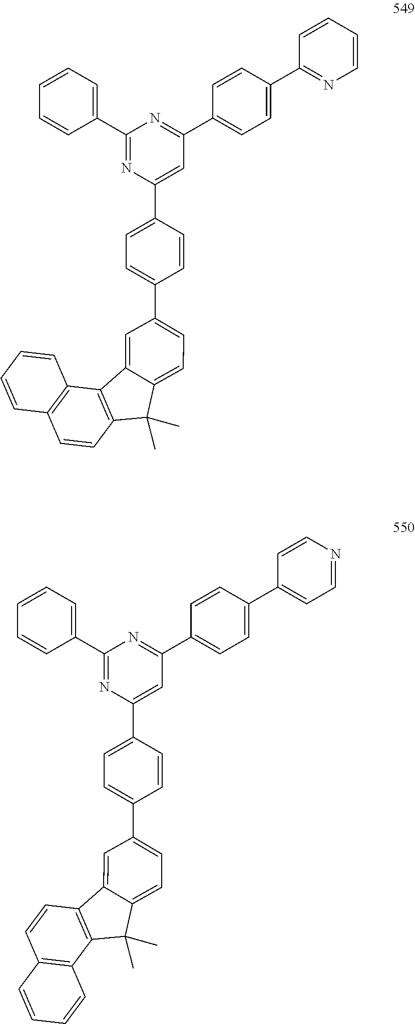 [SMILES: CC1(C)c2ccc(-c3ccc(-c4cc(-c5ccc(-c6ccccn6)cc5)nc(-c5ccccc5)n4)cc3)cc2-c2c1ccc1ccccc21 ; CC1(C)c2ccc(-c3ccc(-c4cc(-c5ccc(-c6ccncc6)cc5)nc(-c5ccccc5)n4)cc3)cc2-c2ccc3ccccc3c21]

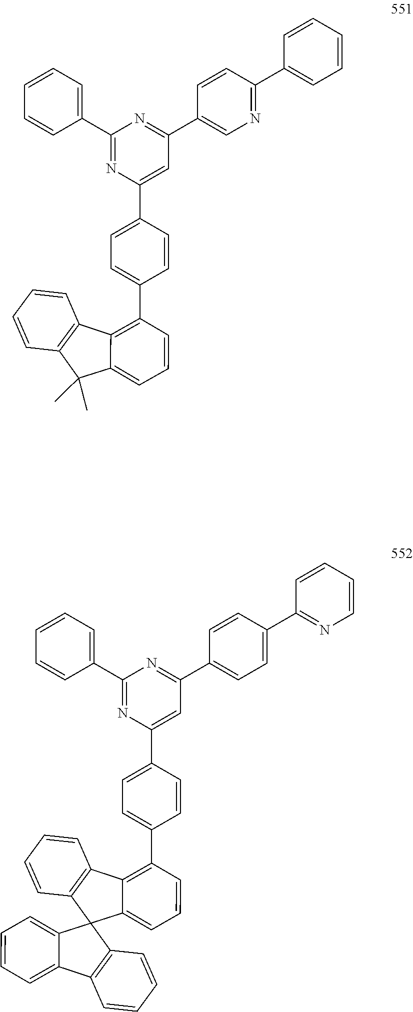 [SMILES: CC1(C)c2ccccc2-c2c(-c3ccc(-c4cc(-c5ccc(-c6ccccc6)nc5)nc(-c5ccccc5)n4)cc3)cccc21 ; c1ccc(-c2nc(-c3ccc(-c4ccccn4)cc3)cc(-c3ccc(-c4cccc5c4-c4ccccc4C54c5ccccc5-c5ccccc54)cc3)n2)cc1]

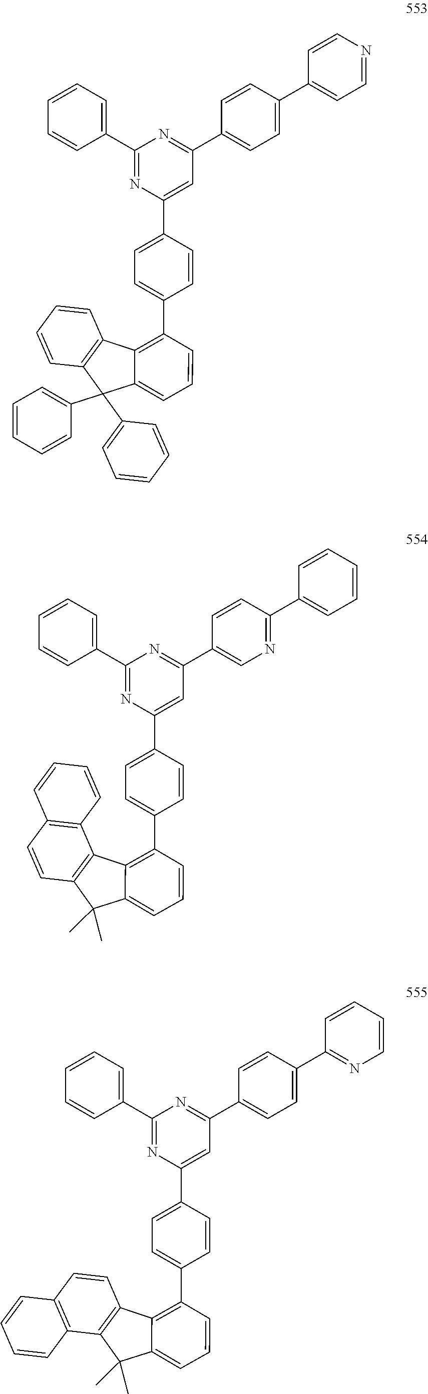 [SMILES: c1ccc(-c2nc(-c3ccc(-c4ccncc4)cc3)cc(-c3ccc(-c4cccc5c4-c4ccccc4C5(c4ccccc4)c4ccccc4)cc3)n2)cc1 ; CC1(C)c2cccc(-c3ccc(-c4cc(-c5ccc(-c6ccccc6)nc5)nc(-c5ccccc5)n4)cc3)c2-c2c1ccc1ccccc21 ; CC1(C)c2cccc(-c3ccc(-c4cc(-c5ccc(-c6ccccn6)cc5)nc(-c5ccccc5)n4)cc3)c2-c2ccc3ccccc3c21]

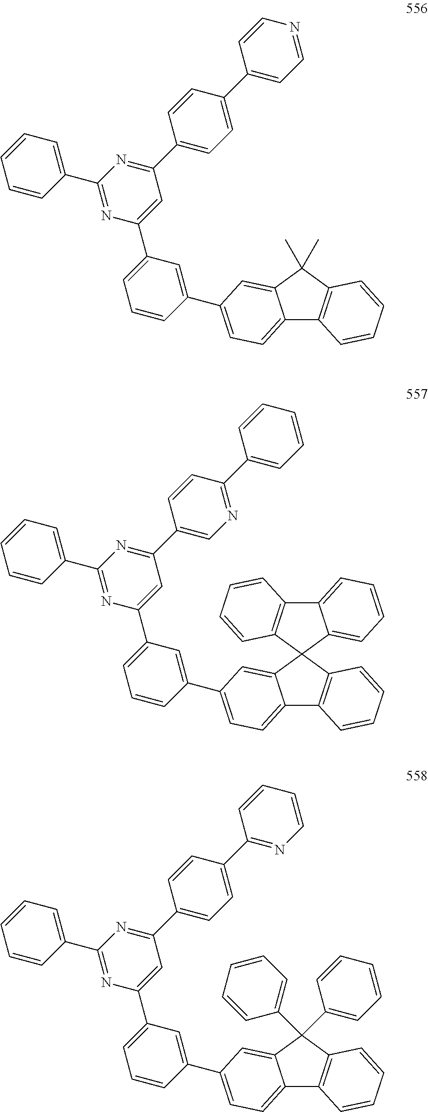 [SMILES: CC1(C)c2ccccc2-c2ccc(-c3cccc(-c4cc(-c5ccc(-c6ccncc6)cc5)nc(-c5ccccc5)n4)c3)cc21 ; c1ccc(-c2ccc(-c3cc(-c4cccc(-c5ccc6c(c5)C5(c7ccccc7-c7ccccc75)c5ccccc5-6)c4)nc(-c4ccccc4)n3)cn2)cc1 ; c1ccc(-c2nc(-c3ccc(-c4ccccn4)cc3)cc(-c3cccc(-c4ccc5c(c4)C(c4ccccc4)(c4ccccc4)c4ccccc4-5)c3)n2)cc1]

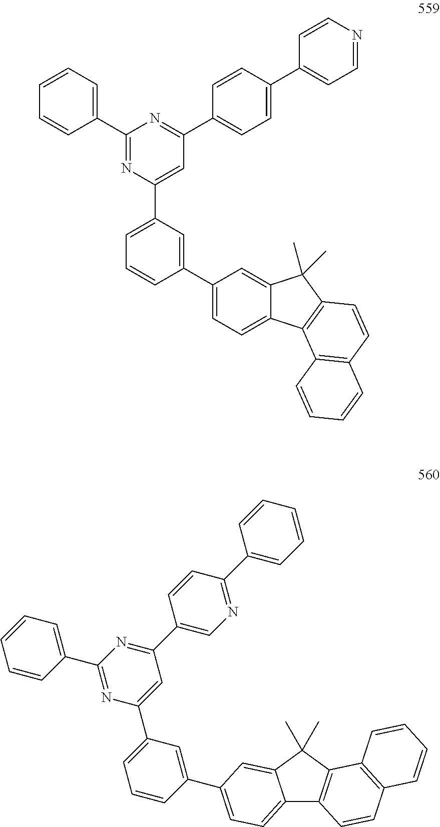 [SMILES: CC1(C)c2cc(-c3cccc(-c4cc(-c5ccc(-c6ccncc6)cc5)nc(-c5ccccc5)n4)c3)ccc2-c2c1ccc1ccccc21 ; CC1(C)c2cc(-c3cccc(-c4cc(-c5ccc(-c6ccccc6)nc5)nc(-c5ccccc5)n4)c3)ccc2-c2ccc3ccccc3c21]

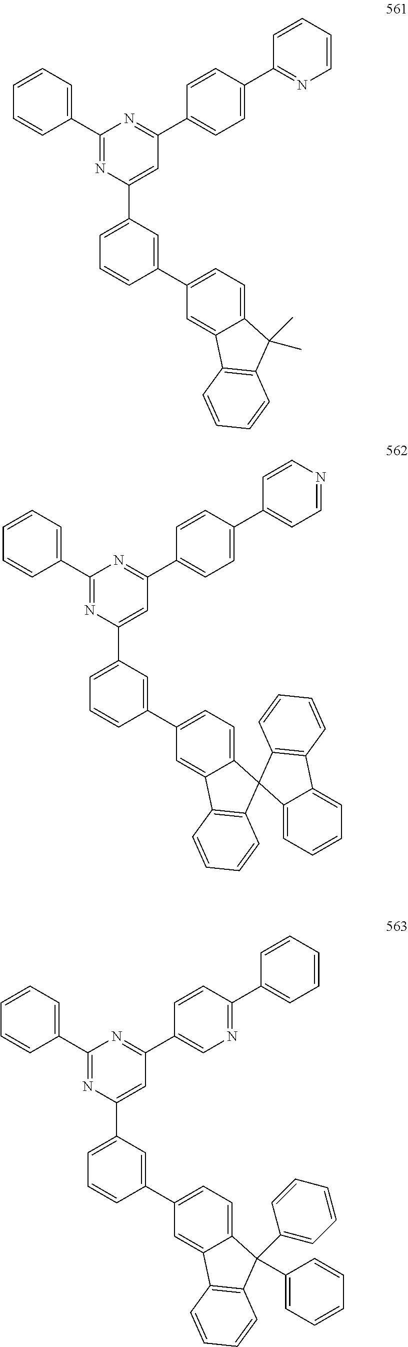 [SMILES: CC1(C)c2ccccc2-c2cc(-c3cccc(-c4cc(-c5ccc(-c6ccccn6)cc5)nc(-c5ccccc5)n4)c3)ccc21 ; c1ccc(-c2nc(-c3ccc(-c4ccncc4)cc3)cc(-c3cccc(-c4ccc5c(c4)-c4ccccc4C54c5ccccc5-c5ccccc54)c3)n2)cc1 ; c1ccc(-c2ccc(-c3cc(-c4cccc(-c5ccc6c(c5)-c5ccccc5C6(c5ccccc5)c5ccccc5)c4)nc(-c4ccccc4)n3)cn2)cc1]

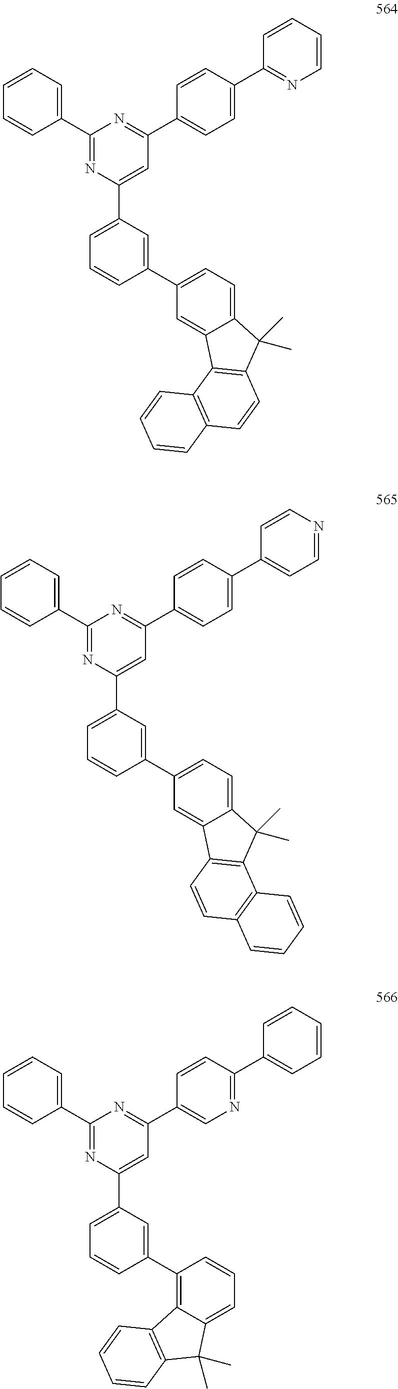 [SMILES: CC1(C)c2ccc(-c3cccc(-c4cc(-c5ccc(-c6ccccn6)cc5)nc(-c5ccccc5)n4)c3)cc2-c2c1ccc1ccccc21 ; CC1(C)c2ccc(-c3cccc(-c4cc(-c5ccc(-c6ccncc6)cc5)nc(-c5ccccc5)n4)c3)cc2-c2ccc3ccccc3c21 ; CC1(C)c2ccccc2-c2c(-c3cccc(-c4cc(-c5ccc(-c6ccccc6)nc5)nc(-c5ccccc5)n4)c3)cccc21]

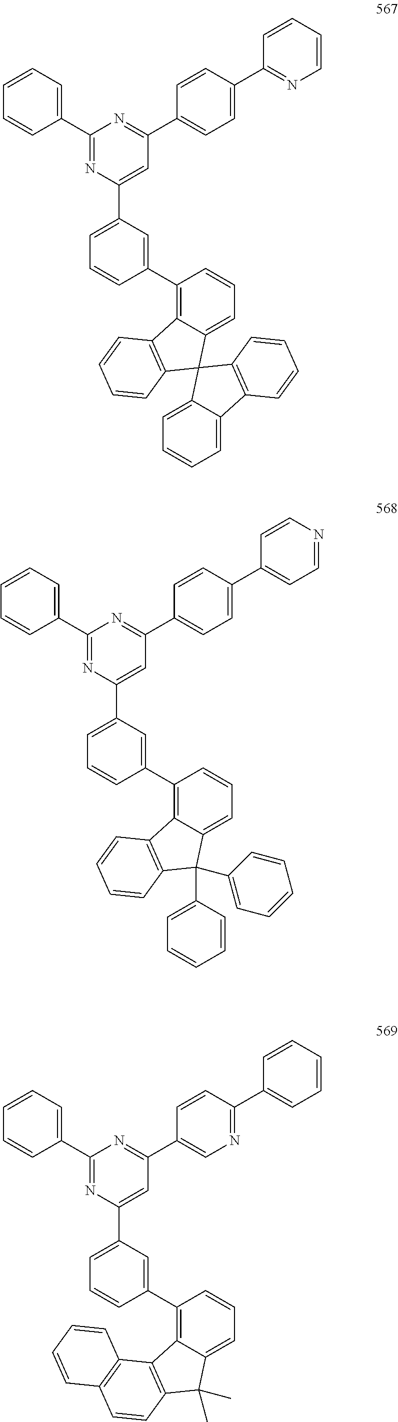 [SMILES: c1ccc(-c2nc(-c3ccc(-c4ccccn4)cc3)cc(-c3cccc(-c4cccc5c4-c4ccccc4C54c5ccccc5-c5ccccc54)c3)n2)cc1 ; c1ccc(-c2nc(-c3ccc(-c4ccncc4)cc3)cc(-c3cccc(-c4cccc5c4-c4ccccc4C5(c4ccccc4)c4ccccc4)c3)n2)cc1 ; CC1(C)c2cccc(-c3cccc(-c4cc(-c5ccc(-c6ccccc6)nc5)nc(-c5ccccc5)n4)c3)c2-c2c1ccc1ccccc21]

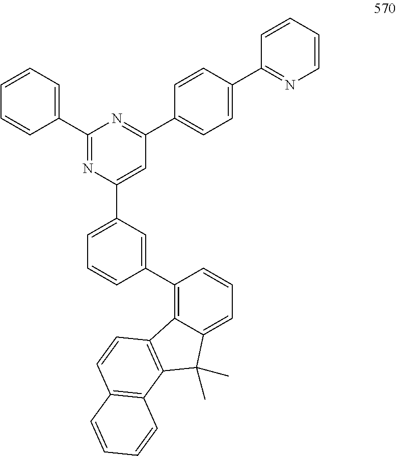 [SMILES: CC1(C)c2cccc(-c3cccc(-c4cc(-c5ccc(-c6ccccn6)cc5)nc(-c5ccccc5)n4)c3)c2-c2ccc3ccccc3c21]

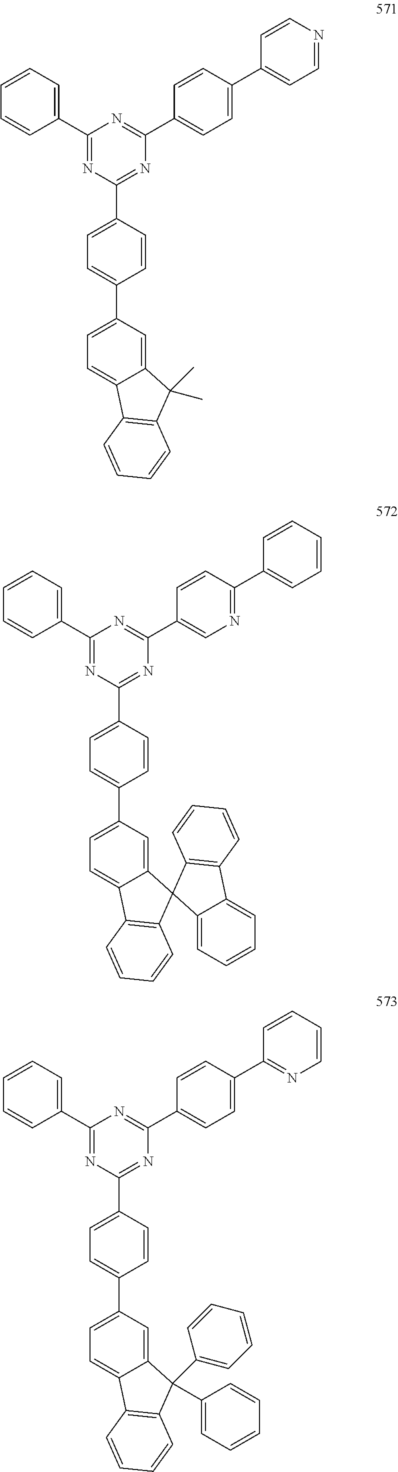 [SMILES: CC1(C)c2ccccc2-c2ccc(-c3ccc(-c4nc(-c5ccccc5)nc(-c5ccc(-c6ccncc6)cc5)n4)cc3)cc21 ; c1ccc(-c2ccc(-c3nc(-c4ccccc4)nc(-c4ccc(-c5ccc6c(c5)C5(c7ccccc7-c7ccccc75)c5ccccc5-6)cc4)n3)cn2)cc1 ; c1ccc(-c2nc(-c3ccc(-c4ccc5c(c4)C(c4ccccc4)(c4ccccc4)c4ccccc4-5)cc3)nc(-c3ccc(-c4ccccn4)cc3)n2)cc1]

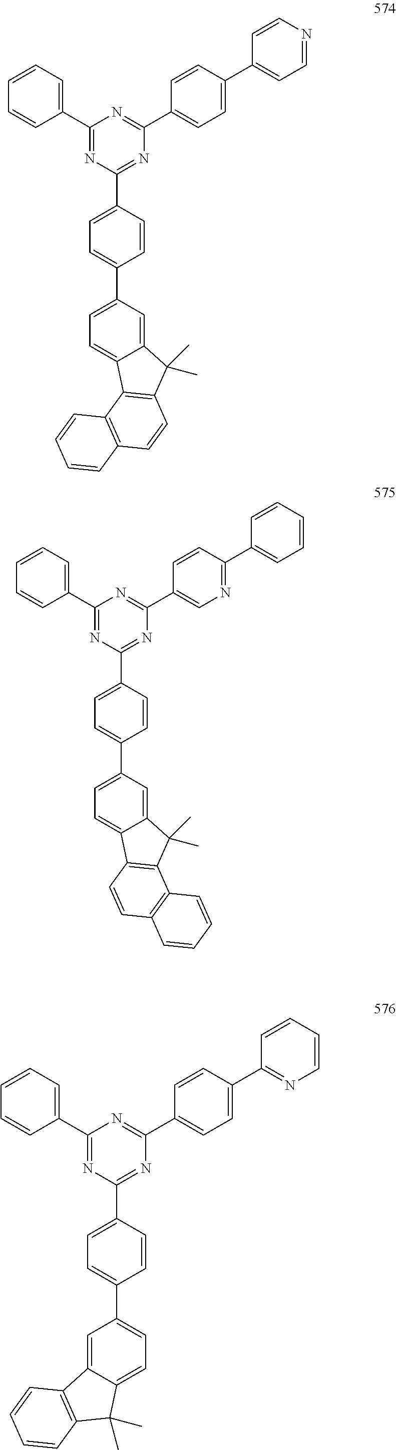 [SMILES: CC1(C)c2cc(-c3ccc(-c4nc(-c5ccccc5)nc(-c5ccc(-c6ccncc6)cc5)n4)cc3)ccc2-c2c1ccc1ccccc21 ; CC1(C)c2cc(-c3ccc(-c4nc(-c5ccccc5)nc(-c5ccc(-c6ccccc6)nc5)n4)cc3)ccc2-c2ccc3ccccc3c21 ; CC1(C)c2ccccc2-c2cc(-c3ccc(-c4nc(-c5ccccc5)nc(-c5ccc(-c6ccccn6)cc5)n4)cc3)ccc21]

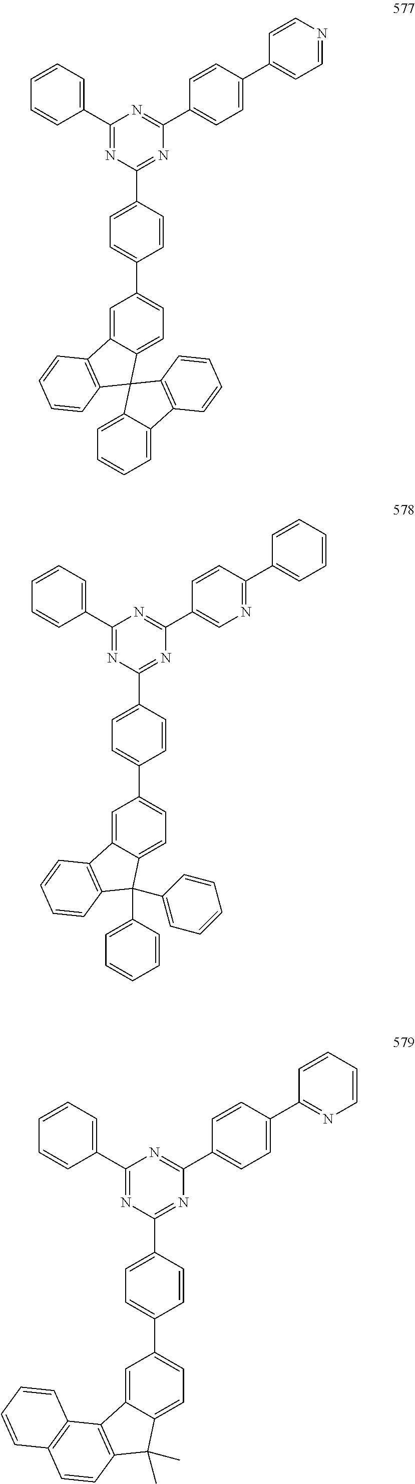 [SMILES: c1ccc(-c2nc(-c3ccc(-c4ccncc4)cc3)nc(-c3ccc(-c4ccc5c(c4)-c4ccccc4C54c5ccccc5-c5ccccc54)cc3)n2)cc1 ; c1ccc(-c2ccc(-c3nc(-c4ccccc4)nc(-c4ccc(-c5ccc6c(c5)-c5ccccc5C6(c5ccccc5)c5ccccc5)cc4)n3)cn2)cc1 ; CC1(C)c2ccc(-c3ccc(-c4nc(-c5ccccc5)nc(-c5ccc(-c6ccccn6)cc5)n4)cc3)cc2-c2c1ccc1ccccc21]

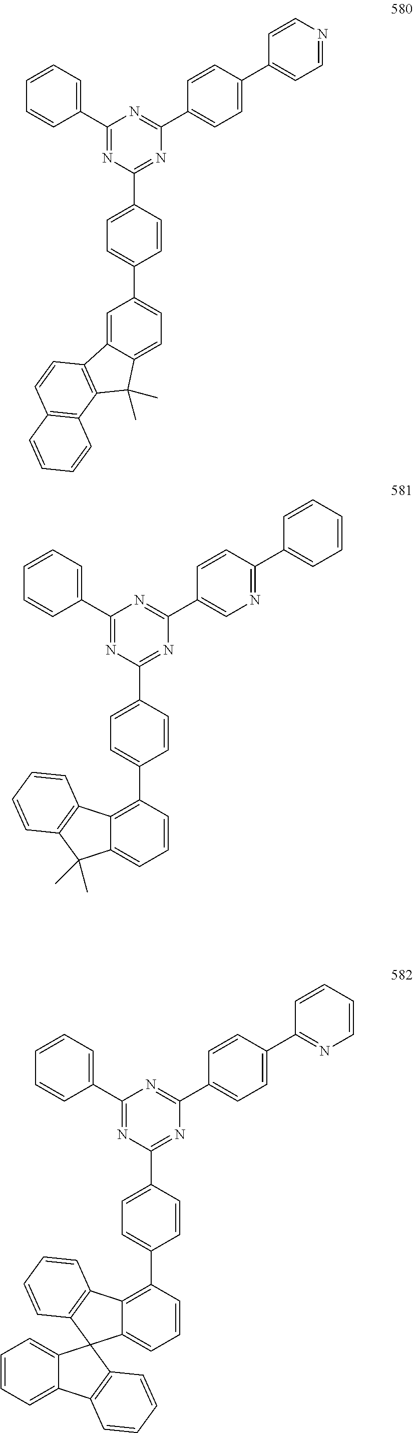 [SMILES: CC1(C)c2ccc(-c3ccc(-c4nc(-c5ccccc5)nc(-c5ccc(-c6ccncc6)cc5)n4)cc3)cc2-c2ccc3ccccc3c21 ; CC1(C)c2ccccc2-c2c(-c3ccc(-c4nc(-c5ccccc5)nc(-c5ccc(-c6ccccc6)nc5)n4)cc3)cccc21 ; c1ccc(-c2nc(-c3ccc(-c4ccccn4)cc3)nc(-c3ccc(-c4cccc5c4-c4ccccc4C54c5ccccc5-c5ccccc54)cc3)n2)cc1]

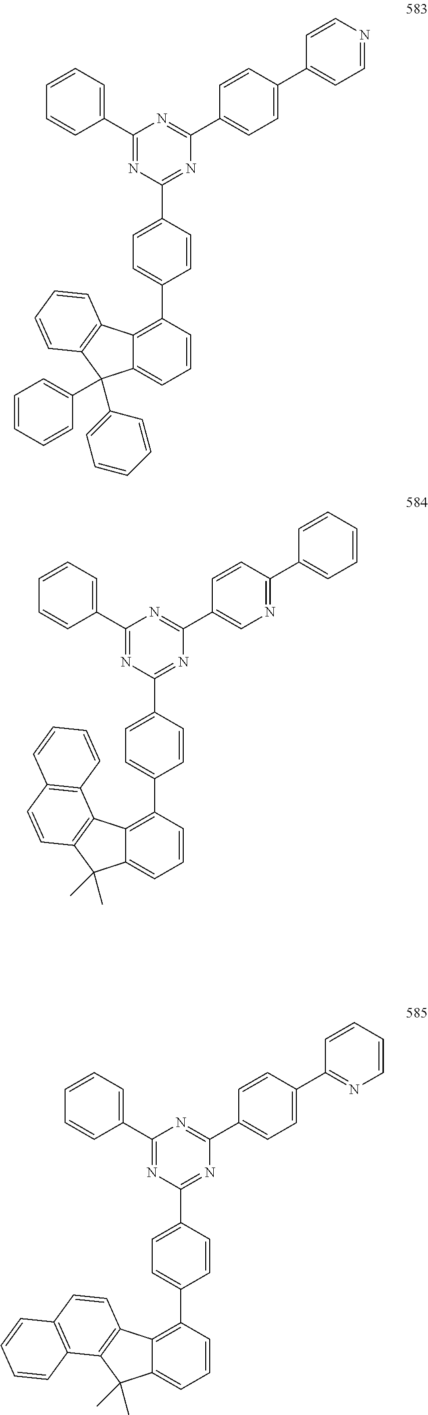 [SMILES: c1ccc(-c2nc(-c3ccc(-c4ccncc4)cc3)nc(-c3ccc(-c4cccc5c4-c4ccccc4C5(c4ccccc4)c4ccccc4)cc3)n2)cc1 ; CC1(C)c2cccc(-c3ccc(-c4nc(-c5ccccc5)nc(-c5ccc(-c6ccccc6)nc5)n4)cc3)c2-c2c1ccc1ccccc21 ; CC1(C)c2cccc(-c3ccc(-c4nc(-c5ccccc5)nc(-c5ccc(-c6ccccn6)cc5)n4)cc3)c2-c2ccc3ccccc3c21]

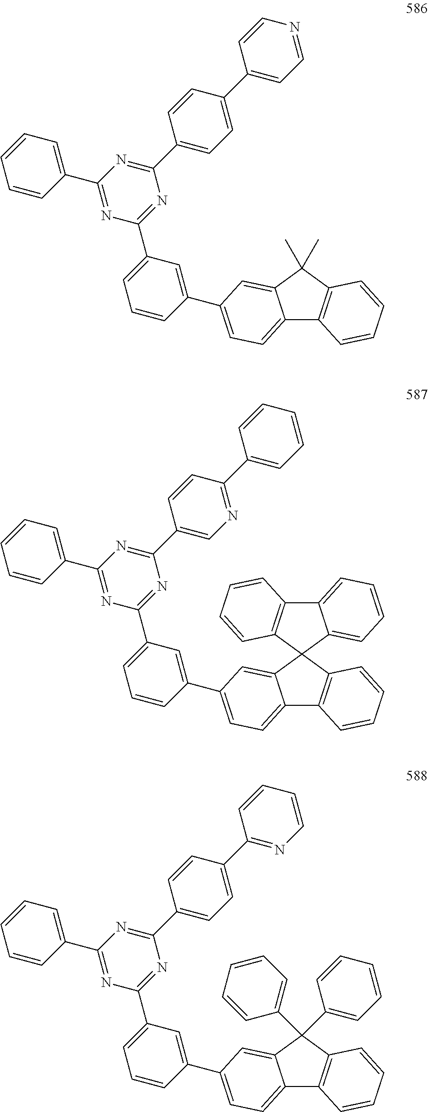 [SMILES: CC1(C)c2ccccc2-c2ccc(-c3cccc(-c4nc(-c5ccccc5)nc(-c5ccc(-c6ccncc6)cc5)n4)c3)cc21 ; c1ccc(-c2ccc(-c3nc(-c4ccccc4)nc(-c4cccc(-c5ccc6c(c5)C5(c7ccccc7-c7ccccc75)c5ccccc5-6)c4)n3)cn2)cc1 ; c1ccc(-c2nc(-c3ccc(-c4ccccn4)cc3)nc(-c3cccc(-c4ccc5c(c4)C(c4ccccc4)(c4ccccc4)c4ccccc4-5)c3)n2)cc1]

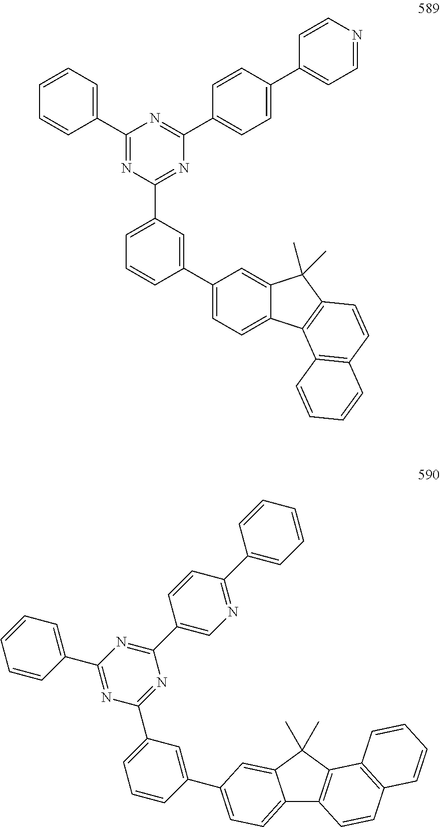 [SMILES: CC1(C)c2cc(-c3cccc(-c4nc(-c5ccccc5)nc(-c5ccc(-c6ccncc6)cc5)n4)c3)ccc2-c2c1ccc1ccccc21 ; CC1(C)c2cc(-c3cccc(-c4nc(-c5ccccc5)nc(-c5ccc(-c6ccccc6)nc5)n4)c3)ccc2-c2ccc3ccccc3c21]

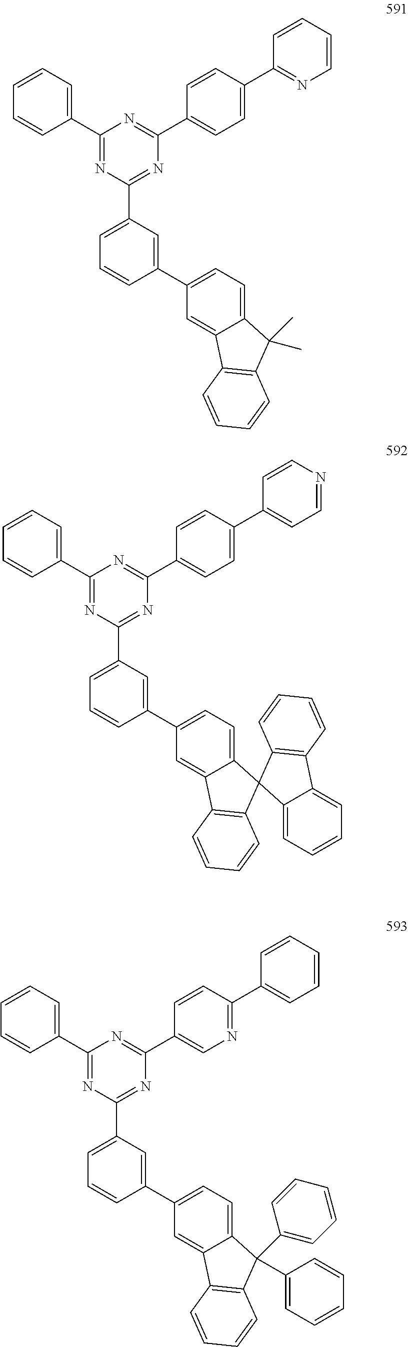 [SMILES: CC1(C)c2ccccc2-c2cc(-c3cccc(-c4nc(-c5ccccc5)nc(-c5ccc(-c6ccccn6)cc5)n4)c3)ccc21 ; c1ccc(-c2nc(-c3ccc(-c4ccncc4)cc3)nc(-c3cccc(-c4ccc5c(c4)-c4ccccc4C54c5ccccc5-c5ccccc54)c3)n2)cc1 ; c1ccc(-c2ccc(-c3nc(-c4ccccc4)nc(-c4cccc(-c5ccc6c(c5)-c5ccccc5C6(c5ccccc5)c5ccccc5)c4)n3)cn2)cc1]

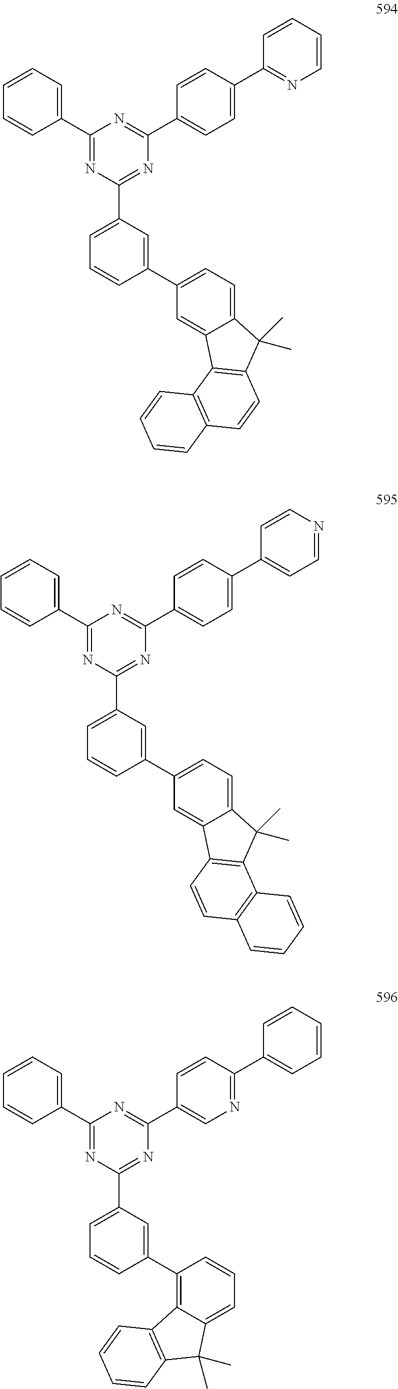 [SMILES: CC1(C)c2ccc(-c3cccc(-c4nc(-c5ccccc5)nc(-c5ccc(-c6ccccn6)cc5)n4)c3)cc2-c2c1ccc1ccccc21 ; CC1(C)c2ccc(-c3cccc(-c4nc(-c5ccccc5)nc(-c5ccc(-c6ccncc6)cc5)n4)c3)cc2-c2ccc3ccccc3c21 ; CC1(C)c2ccccc2-c2c(-c3cccc(-c4nc(-c5ccccc5)nc(-c5ccc(-c6ccccc6)nc5)n4)c3)cccc21]

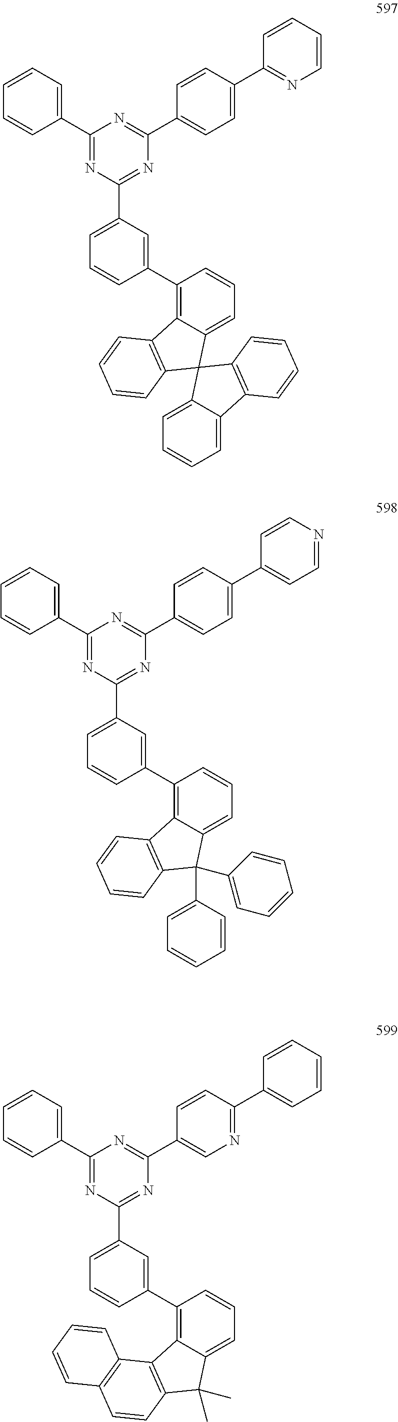 [SMILES: c1ccc(-c2nc(-c3ccc(-c4ccccn4)cc3)nc(-c3cccc(-c4cccc5c4-c4ccccc4C54c5ccccc5-c5ccccc54)c3)n2)cc1 ; c1ccc(-c2nc(-c3ccc(-c4ccncc4)cc3)nc(-c3cccc(-c4cccc5c4-c4ccccc4C5(c4ccccc4)c4ccccc4)c3)n2)cc1 ; CC1(C)c2cccc(-c3cccc(-c4nc(-c5ccccc5)nc(-c5ccc(-c6ccccc6)nc5)n4)c3)c2-c2c1ccc1ccccc21]

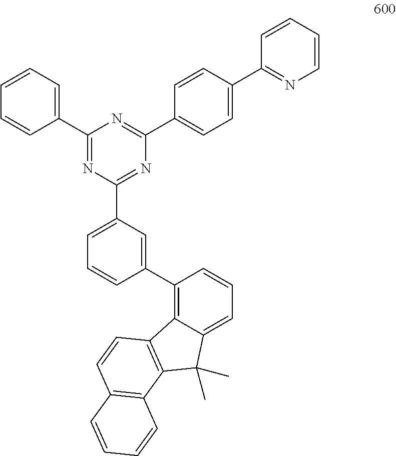 [SMILES: CC1(C)c2cccc(-c3cccc(-c4nc(-c5ccccc5)nc(-c5ccc(-c6ccccn6)cc5)n4)c3)c2-c2ccc3ccccc3c21]

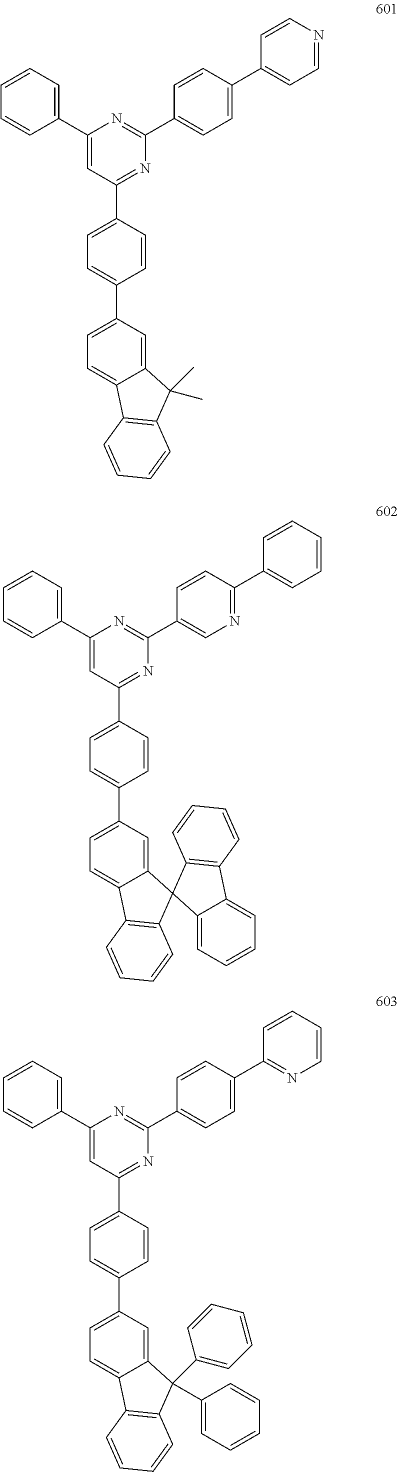 [SMILES: CC1(C)c2ccccc2-c2ccc(-c3ccc(-c4cc(-c5ccccc5)nc(-c5ccc(-c6ccncc6)cc5)n4)cc3)cc21 ; c1ccc(-c2ccc(-c3nc(-c4ccccc4)cc(-c4ccc(-c5ccc6c(c5)C5(c7ccccc7-c7ccccc75)c5ccccc5-6)cc4)n3)cn2)cc1 ; c1ccc(-c2cc(-c3ccc(-c4ccc5c(c4)C(c4ccccc4)(c4ccccc4)c4ccccc4-5)cc3)nc(-c3ccc(-c4ccccn4)cc3)n2)cc1]

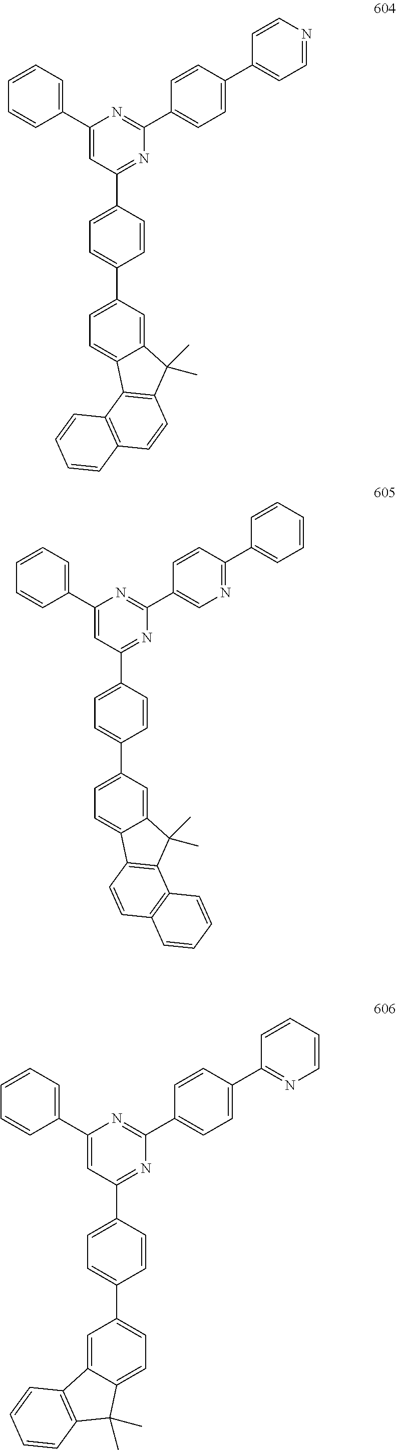 [SMILES: CC1(C)c2cc(-c3ccc(-c4cc(-c5ccccc5)nc(-c5ccc(-c6ccncc6)cc5)n4)cc3)ccc2-c2c1ccc1ccccc21 ; CC1(C)c2cc(-c3ccc(-c4cc(-c5ccccc5)nc(-c5ccc(-c6ccccc6)nc5)n4)cc3)ccc2-c2ccc3ccccc3c21 ; CC1(C)c2ccccc2-c2cc(-c3ccc(-c4cc(-c5ccccc5)nc(-c5ccc(-c6ccccn6)cc5)n4)cc3)ccc21]

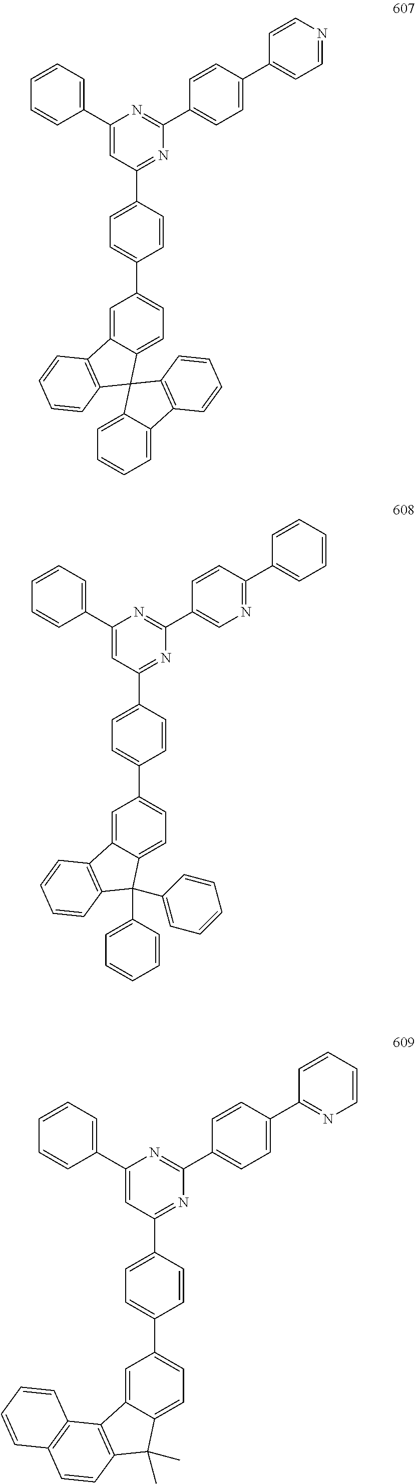 [SMILES: c1ccc(-c2cc(-c3ccc(-c4ccc5c(c4)-c4ccccc4C54c5ccccc5-c5ccccc54)cc3)nc(-c3ccc(-c4ccncc4)cc3)n2)cc1 ; c1ccc(-c2ccc(-c3nc(-c4ccccc4)cc(-c4ccc(-c5ccc6c(c5)-c5ccccc5C6(c5ccccc5)c5ccccc5)cc4)n3)cn2)cc1 ; CC1(C)c2ccc(-c3ccc(-c4cc(-c5ccccc5)nc(-c5ccc(-c6ccccn6)cc5)n4)cc3)cc2-c2c1ccc1ccccc21]

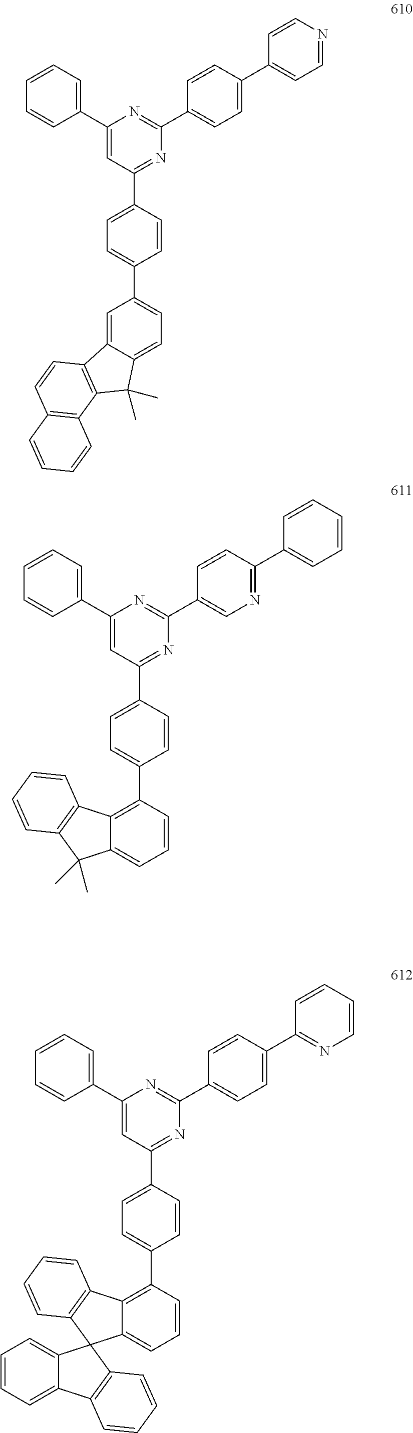 [SMILES: CC1(C)c2ccc(-c3ccc(-c4cc(-c5ccccc5)nc(-c5ccc(-c6ccncc6)cc5)n4)cc3)cc2-c2ccc3ccccc3c21 ; CC1(C)c2ccccc2-c2c(-c3ccc(-c4cc(-c5ccccc5)nc(-c5ccc(-c6ccccc6)nc5)n4)cc3)cccc21 ; c1ccc(-c2cc(-c3ccc(-c4cccc5c4-c4ccccc4C54c5ccccc5-c5ccccc54)cc3)nc(-c3ccc(-c4ccccn4)cc3)n2)cc1]

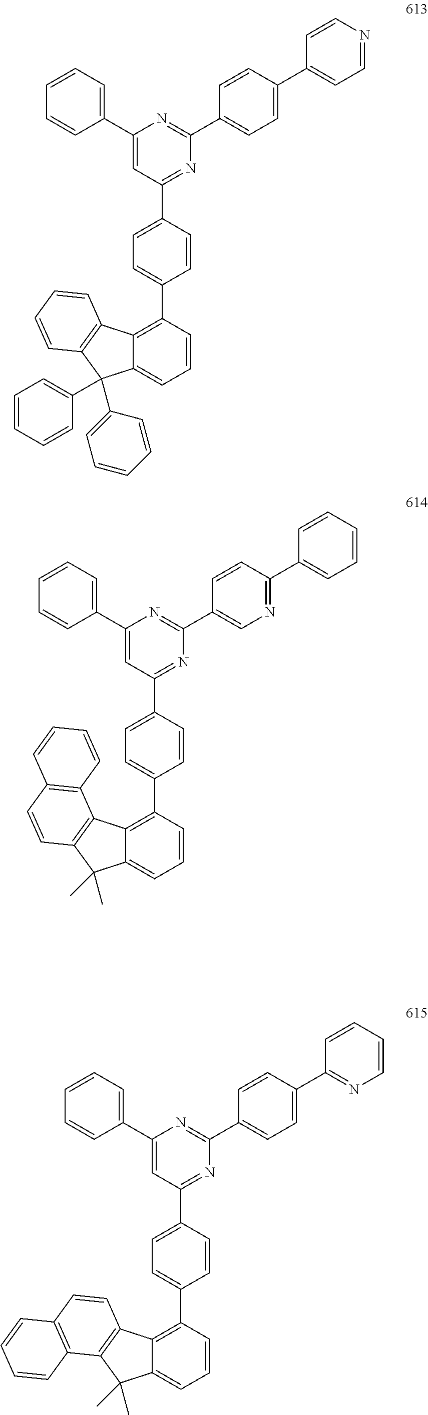 [SMILES: c1ccc(-c2cc(-c3ccc(-c4cccc5c4-c4ccccc4C5(c4ccccc4)c4ccccc4)cc3)nc(-c3ccc(-c4ccncc4)cc3)n2)cc1 ; CC1(C)c2cccc(-c3ccc(-c4cc(-c5ccccc5)nc(-c5ccc(-c6ccccc6)nc5)n4)cc3)c2-c2c1ccc1ccccc21 ; CC1(C)c2cccc(-c3ccc(-c4cc(-c5ccccc5)nc(-c5ccc(-c6ccccn6)cc5)n4)cc3)c2-c2ccc3ccccc3c21]

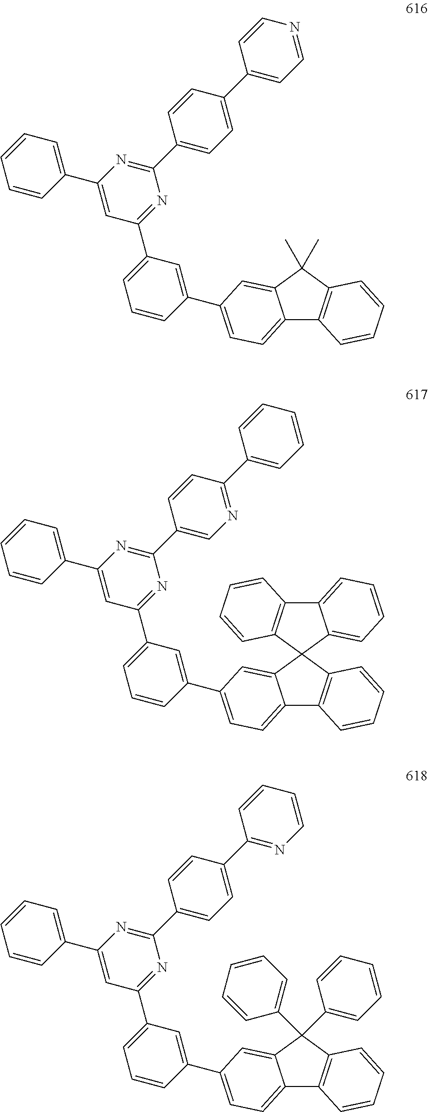 [SMILES: CC1(C)c2ccccc2-c2ccc(-c3cccc(-c4cc(-c5ccccc5)nc(-c5ccc(-c6ccncc6)cc5)n4)c3)cc21 ; c1ccc(-c2ccc(-c3nc(-c4ccccc4)cc(-c4cccc(-c5ccc6c(c5)C5(c7ccccc7-c7ccccc75)c5ccccc5-6)c4)n3)cn2)cc1 ; c1ccc(-c2cc(-c3cccc(-c4ccc5c(c4)C(c4ccccc4)(c4ccccc4)c4ccccc4-5)c3)nc(-c3ccc(-c4ccccn4)cc3)n2)cc1]

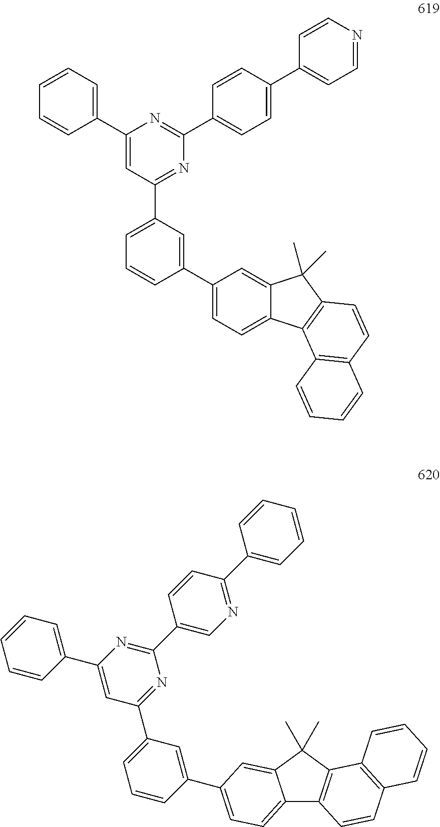 [SMILES: CC1(C)c2cc(-c3cccc(-c4cc(-c5ccccc5)nc(-c5ccc(-c6ccncc6)cc5)n4)c3)ccc2-c2c1ccc1ccccc21 ; CC1(C)c2cc(-c3cccc(-c4cc(-c5ccccc5)nc(-c5ccc(-c6ccccc6)nc5)n4)c3)ccc2-c2ccc3ccccc3c21]

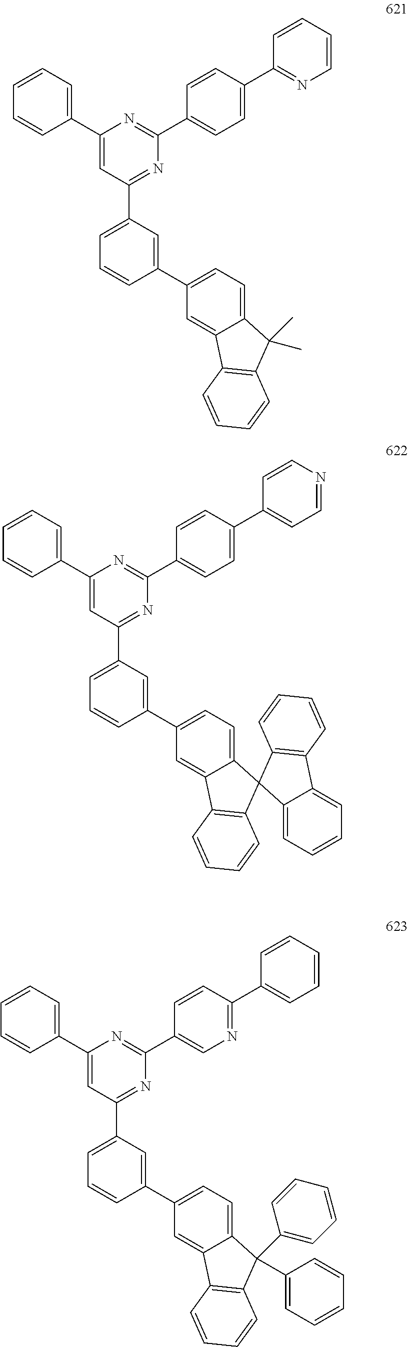 [SMILES: CC1(C)c2ccccc2-c2cc(-c3cccc(-c4cc(-c5ccccc5)nc(-c5ccc(-c6ccccn6)cc5)n4)c3)ccc21 ; c1ccc(-c2cc(-c3cccc(-c4ccc5c(c4)-c4ccccc4C54c5ccccc5-c5ccccc54)c3)nc(-c3ccc(-c4ccncc4)cc3)n2)cc1 ; c1ccc(-c2ccc(-c3nc(-c4ccccc4)cc(-c4cccc(-c5ccc6c(c5)-c5ccccc5C6(c5ccccc5)c5ccccc5)c4)n3)cn2)cc1]

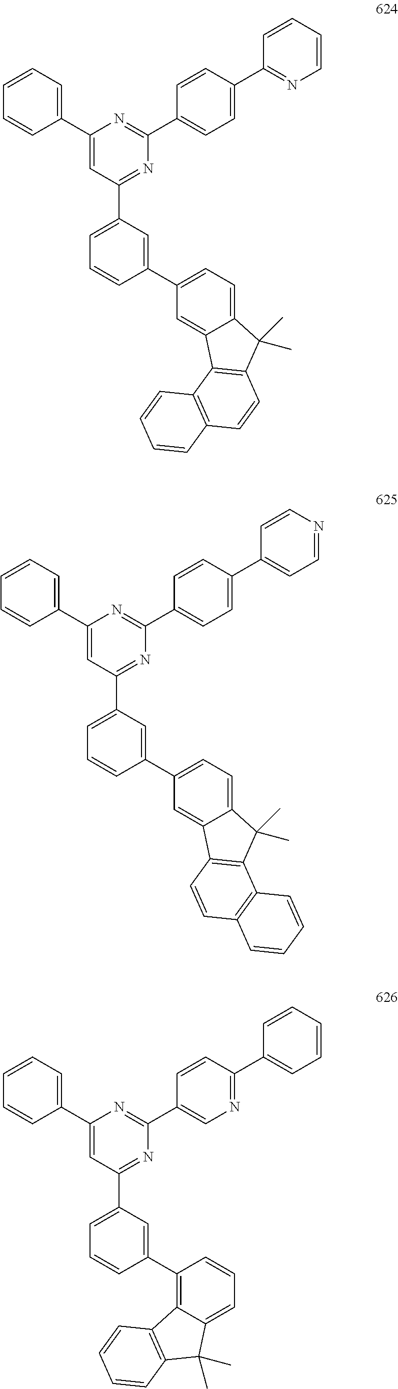 [SMILES: CC1(C)c2ccc(-c3cccc(-c4cc(-c5ccccc5)nc(-c5ccc(-c6ccccn6)cc5)n4)c3)cc2-c2c1ccc1ccccc21 ; CC1(C)c2ccc(-c3cccc(-c4cc(-c5ccccc5)nc(-c5ccc(-c6ccncc6)cc5)n4)c3)cc2-c2ccc3ccccc3c21 ; CC1(C)c2ccccc2-c2c(-c3cccc(-c4cc(-c5ccccc5)nc(-c5ccc(-c6ccccc6)nc5)n4)c3)cccc21]

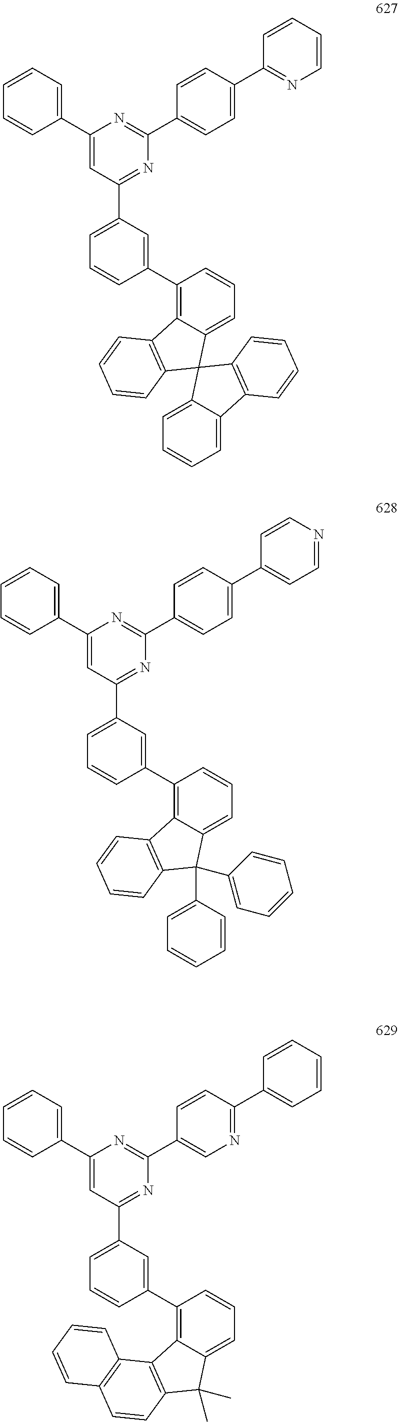 [SMILES: c1ccc(-c2cc(-c3cccc(-c4cccc5c4-c4ccccc4C54c5ccccc5-c5ccccc54)c3)nc(-c3ccc(-c4ccccn4)cc3)n2)cc1 ; c1ccc(-c2cc(-c3cccc(-c4cccc5c4-c4ccccc4C5(c4ccccc4)c4ccccc4)c3)nc(-c3ccc(-c4ccncc4)cc3)n2)cc1 ; CC1(C)c2cccc(-c3cccc(-c4cc(-c5ccccc5)nc(-c5ccc(-c6ccccc6)nc5)n4)c3)c2-c2c1ccc1ccccc21]

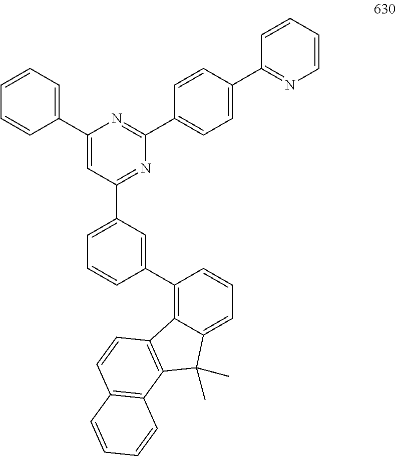 [SMILES: CC1(C)c2cccc(-c3cccc(-c4cc(-c5ccccc5)nc(-c5ccc(-c6ccccn6)cc5)n4)c3)c2-c2ccc3ccccc3c21]

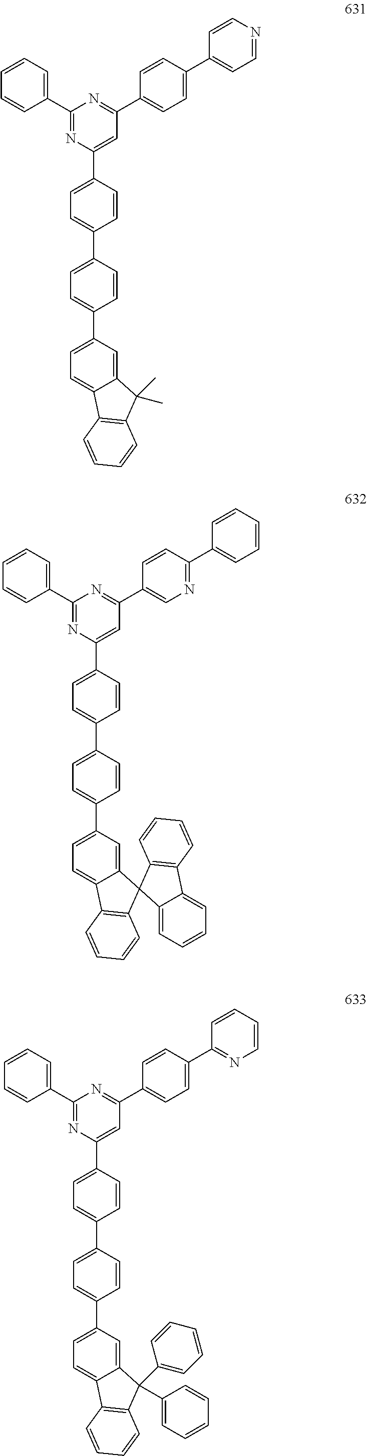 [SMILES: CC1(C)c2ccccc2-c2ccc(-c3ccc(-c4ccc(-c5cc(-c6ccc(-c7ccncc7)cc6)nc(-c6ccccc6)n5)cc4)cc3)cc21 ; c1ccc(-c2ccc(-c3cc(-c4ccc(-c5ccc(-c6ccc7c(c6)C6(c8ccccc8-c8ccccc86)c6ccccc6-7)cc5)cc4)nc(-c4ccccc4)n3)cn2)cc1 ; c1ccc(-c2nc(-c3ccc(-c4ccc(-c5ccc6c(c5)C(c5ccccc5)(c5ccccc5)c5ccccc5-6)cc4)cc3)cc(-c3ccc(-c4ccccn4)cc3)n2)cc1]

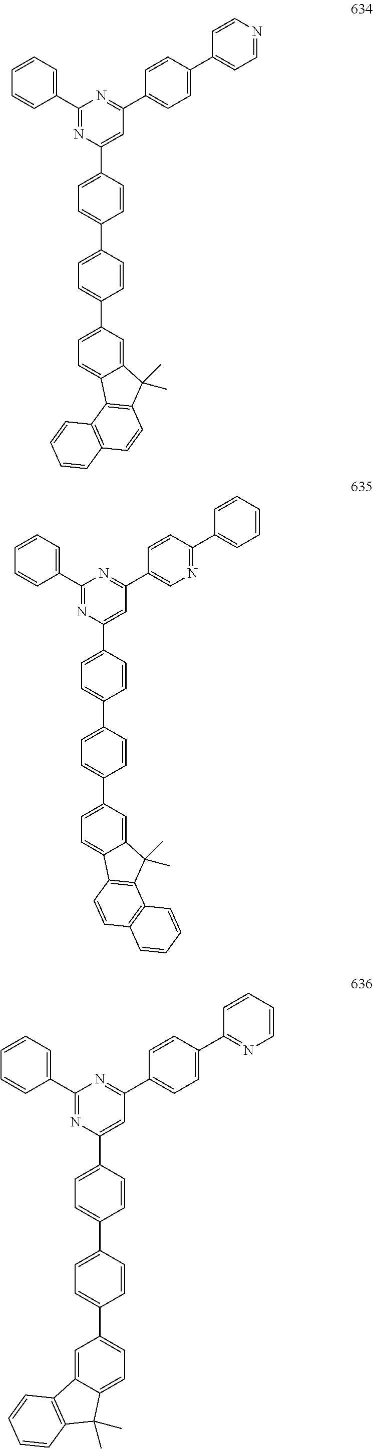 [SMILES: CC1(C)c2cc(-c3ccc(-c4ccc(-c5cc(-c6ccc(-c7ccncc7)cc6)nc(-c6ccccc6)n5)cc4)cc3)ccc2-c2c1ccc1ccccc21 ; CC1(C)c2cc(-c3ccc(-c4ccc(-c5cc(-c6ccc(-c7ccccc7)nc6)nc(-c6ccccc6)n5)cc4)cc3)ccc2-c2ccc3ccccc3c21 ; CC1(C)c2ccccc2-c2cc(-c3ccc(-c4ccc(-c5cc(-c6ccc(-c7ccccn7)cc6)nc(-c6ccccc6)n5)cc4)cc3)ccc21]

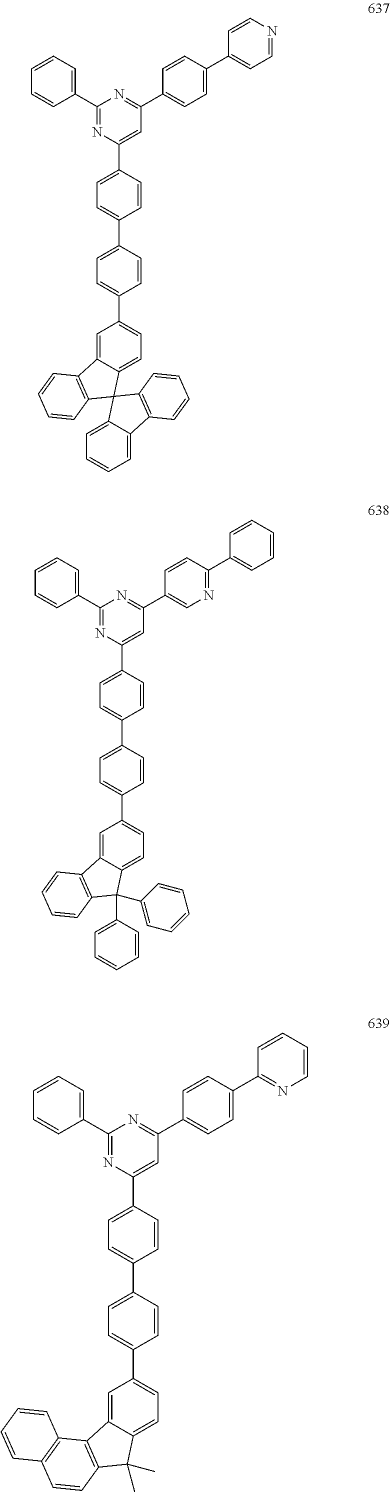 [SMILES: c1ccc(-c2nc(-c3ccc(-c4ccncc4)cc3)cc(-c3ccc(-c4ccc(-c5ccc6c(c5)-c5ccccc5C65c6ccccc6-c6ccccc65)cc4)cc3)n2)cc1 ; c1ccc(-c2ccc(-c3cc(-c4ccc(-c5ccc(-c6ccc7c(c6)-c6ccccc6C7(c6ccccc6)c6ccccc6)cc5)cc4)nc(-c4ccccc4)n3)cn2)cc1 ; CC1(C)c2ccc(-c3ccc(-c4ccc(-c5cc(-c6ccc(-c7ccccn7)cc6)nc(-c6ccccc6)n5)cc4)cc3)cc2-c2c1ccc1ccccc21]

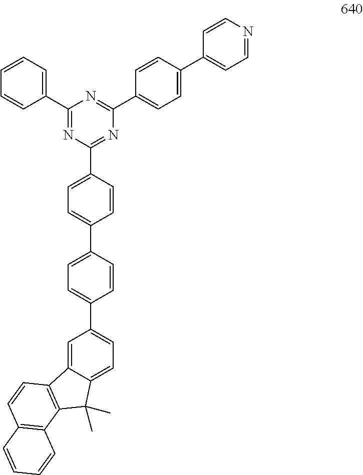 [SMILES: CC1(C)c2ccc(-c3ccc(-c4ccc(-c5nc(-c6ccccc6)nc(-c6ccc(-c7ccncc7)cc6)n5)cc4)cc3)cc2-c2ccc3ccccc3c21]

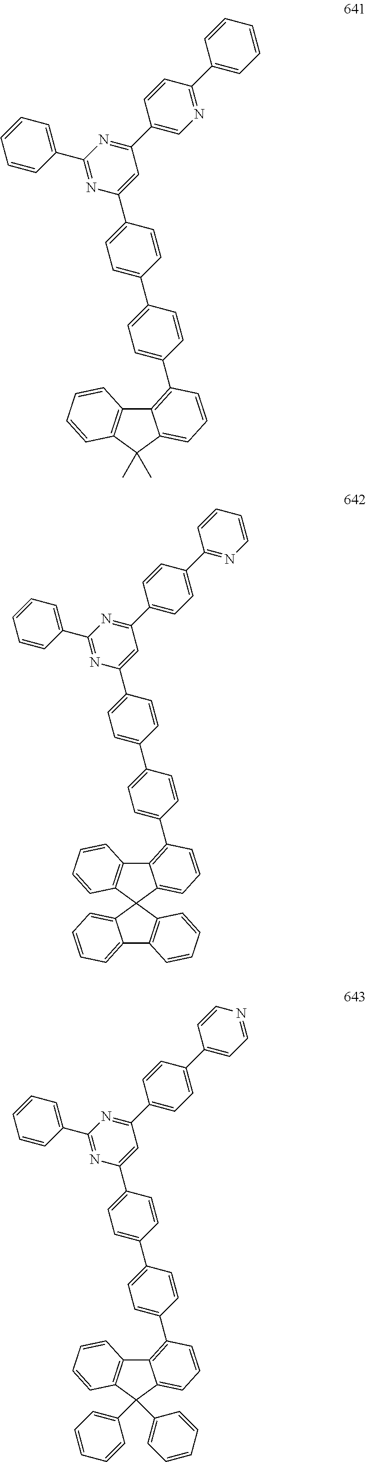 [SMILES: CC1(C)c2ccccc2-c2c(-c3ccc(-c4ccc(-c5cc(-c6ccc(-c7ccccc7)nc6)nc(-c6ccccc6)n5)cc4)cc3)cccc21 ; c1ccc(-c2nc(-c3ccc(-c4ccc(-c5cccc6c5-c5ccccc5C65c6ccccc6-c6ccccc65)cc4)cc3)cc(-c3ccc(-c4ccccn4)cc3)n2)cc1 ; c1ccc(-c2nc(-c3ccc(-c4ccncc4)cc3)cc(-c3ccc(-c4ccc(-c5cccc6c5-c5ccccc5C6(c5ccccc5)c5ccccc5)cc4)cc3)n2)cc1]

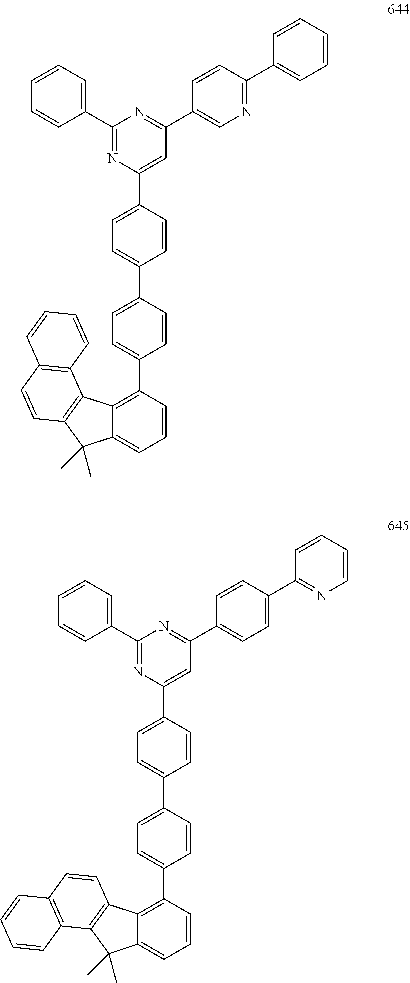 [SMILES: CC1(C)c2cccc(-c3ccc(-c4ccc(-c5cc(-c6ccc(-c7ccccc7)nc6)nc(-c6ccccc6)n5)cc4)cc3)c2-c2c1ccc1ccccc21 ; CC1(C)c2cccc(-c3ccc(-c4ccc(-c5cc(-c6ccc(-c7ccccn7)cc6)nc(-c6ccccc6)n5)cc4)cc3)c2-c2ccc3ccccc3c21]

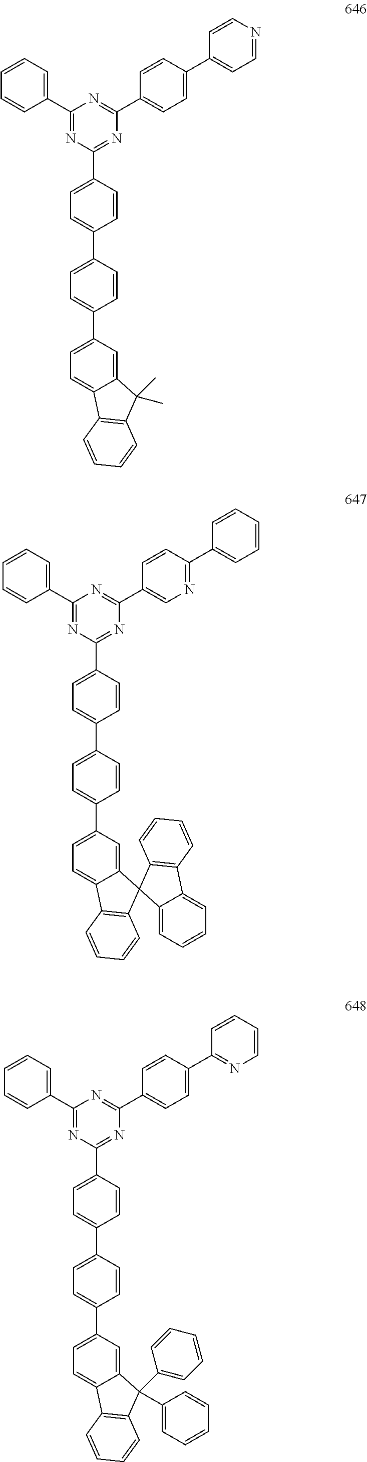 [SMILES: CC1(C)c2ccccc2-c2ccc(-c3ccc(-c4ccc(-c5nc(-c6ccccc6)nc(-c6ccc(-c7ccncc7)cc6)n5)cc4)cc3)cc21 ; c1ccc(-c2ccc(-c3nc(-c4ccccc4)nc(-c4ccc(-c5ccc(-c6ccc7c(c6)C6(c8ccccc8-c8ccccc86)c6ccccc6-7)cc5)cc4)n3)cn2)cc1 ; c1ccc(-c2nc(-c3ccc(-c4ccc(-c5ccc6c(c5)C(c5ccccc5)(c5ccccc5)c5ccccc5-6)cc4)cc3)nc(-c3ccc(-c4ccccn4)cc3)n2)cc1]

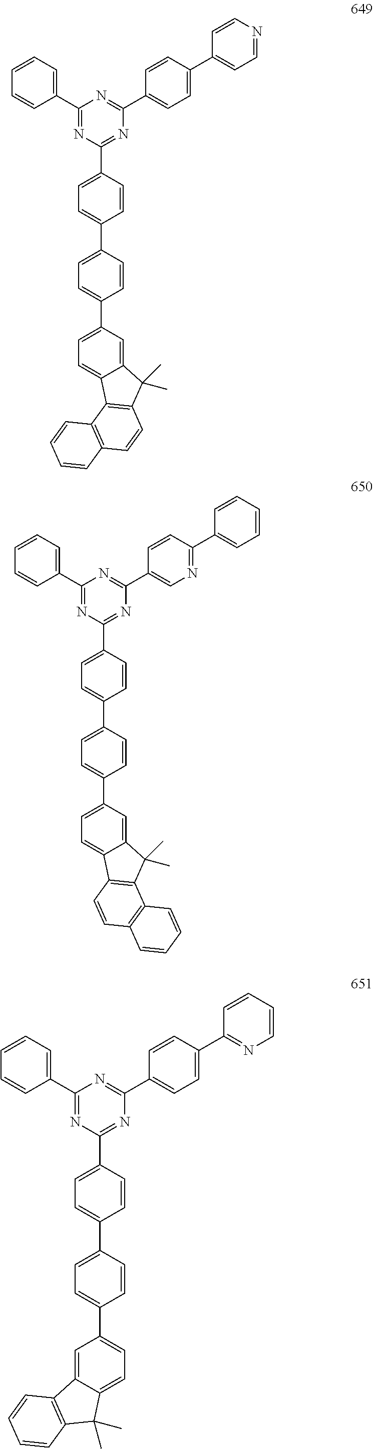 [SMILES: CC1(C)c2cc(-c3ccc(-c4ccc(-c5nc(-c6ccccc6)nc(-c6ccc(-c7ccncc7)cc6)n5)cc4)cc3)ccc2-c2c1ccc1ccccc21 ; CC1(C)c2cc(-c3ccc(-c4ccc(-c5nc(-c6ccccc6)nc(-c6ccc(-c7ccccc7)nc6)n5)cc4)cc3)ccc2-c2ccc3ccccc3c21 ; CC1(C)c2ccccc2-c2cc(-c3ccc(-c4ccc(-c5nc(-c6ccccc6)nc(-c6ccc(-c7ccccn7)cc6)n5)cc4)cc3)ccc21]

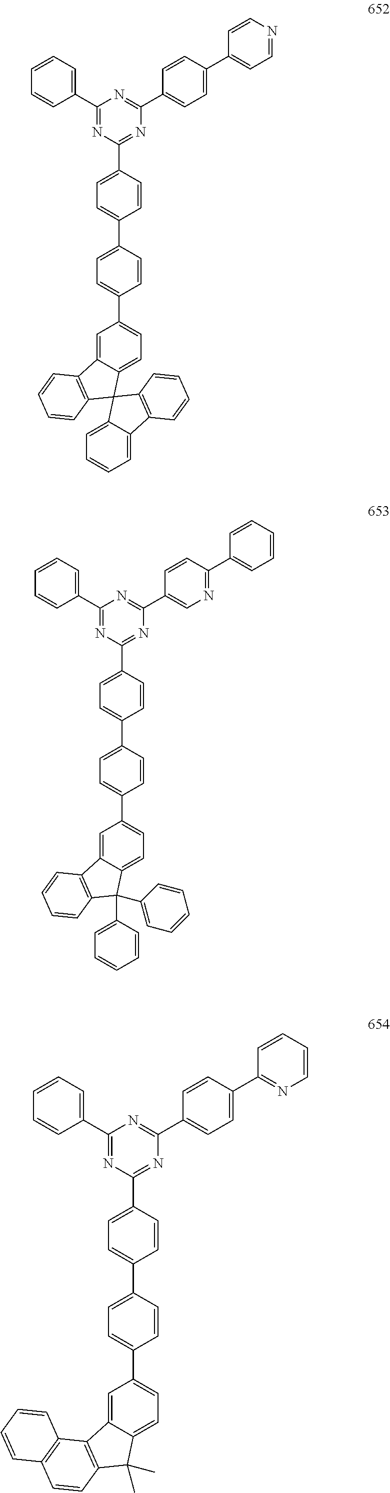 [SMILES: c1ccc(-c2nc(-c3ccc(-c4ccncc4)cc3)nc(-c3ccc(-c4ccc(-c5ccc6c(c5)-c5ccccc5C65c6ccccc6-c6ccccc65)cc4)cc3)n2)cc1 ; c1ccc(-c2ccc(-c3nc(-c4ccccc4)nc(-c4ccc(-c5ccc(-c6ccc7c(c6)-c6ccccc6C7(c6ccccc6)c6ccccc6)cc5)cc4)n3)cn2)cc1 ; CC1(C)c2ccc(-c3ccc(-c4ccc(-c5nc(-c6ccccc6)nc(-c6ccc(-c7ccccn7)cc6)n5)cc4)cc3)cc2-c2c1ccc1ccccc21]

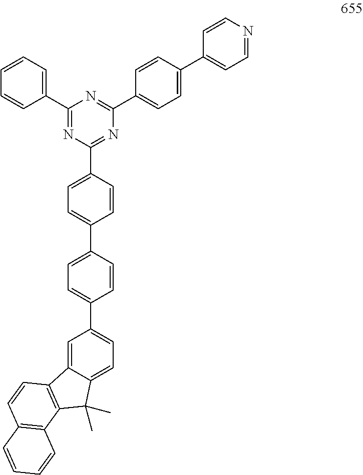 [SMILES: CC1(C)c2ccc(-c3ccc(-c4ccc(-c5nc(-c6ccccc6)nc(-c6ccc(-c7ccncc7)cc6)n5)cc4)cc3)cc2-c2ccc3ccccc3c21]

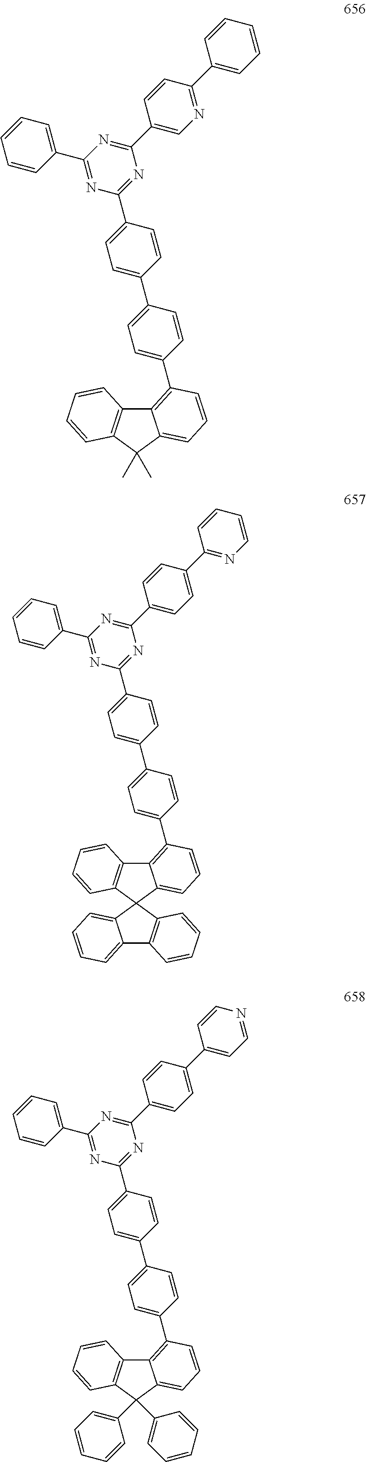 [SMILES: CC1(C)c2ccccc2-c2c(-c3ccc(-c4ccc(-c5nc(-c6ccccc6)nc(-c6ccc(-c7ccccc7)nc6)n5)cc4)cc3)cccc21 ; c1ccc(-c2nc(-c3ccc(-c4ccc(-c5cccc6c5-c5ccccc5C65c6ccccc6-c6ccccc65)cc4)cc3)nc(-c3ccc(-c4ccccn4)cc3)n2)cc1 ; c1ccc(-c2nc(-c3ccc(-c4ccncc4)cc3)nc(-c3ccc(-c4ccc(-c5cccc6c5-c5ccccc5C6(c5ccccc5)c5ccccc5)cc4)cc3)n2)cc1]

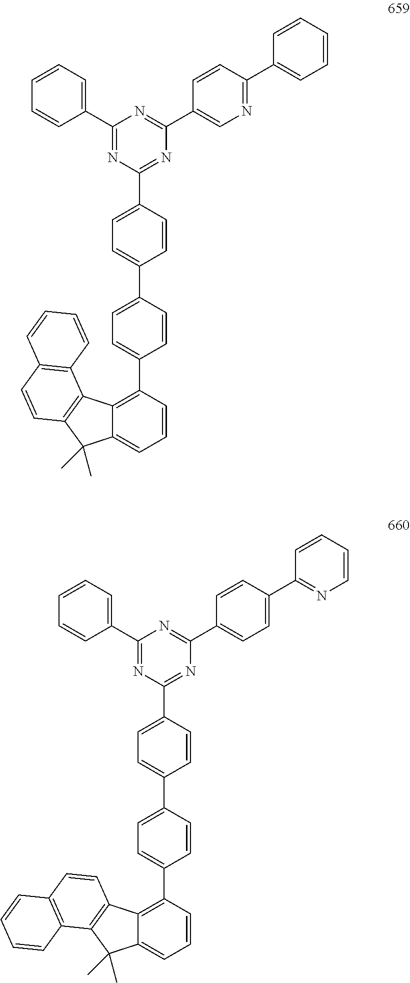 [SMILES: CC1(C)c2cccc(-c3ccc(-c4ccc(-c5nc(-c6ccccc6)nc(-c6ccc(-c7ccccc7)nc6)n5)cc4)cc3)c2-c2c1ccc1ccccc21 ; CC1(C)c2cccc(-c3ccc(-c4ccc(-c5nc(-c6ccccc6)nc(-c6ccc(-c7ccccn7)cc6)n5)cc4)cc3)c2-c2ccc3ccccc3c21]

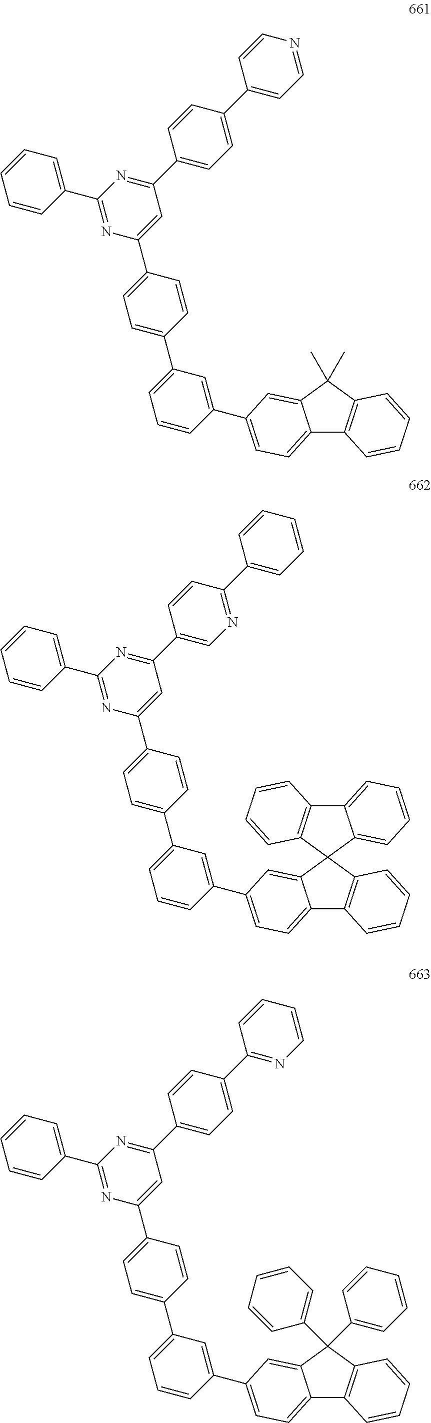 [SMILES: CC1(C)c2ccccc2-c2ccc(-c3cccc(-c4ccc(-c5cc(-c6ccc(-c7ccncc7)cc6)nc(-c6ccccc6)n5)cc4)c3)cc21 ; c1ccc(-c2ccc(-c3cc(-c4ccc(-c5cccc(-c6ccc7c(c6)C6(c8ccccc8-c8ccccc86)c6ccccc6-7)c5)cc4)nc(-c4ccccc4)n3)cn2)cc1 ; c1ccc(-c2nc(-c3ccc(-c4cccc(-c5ccc6c(c5)C(c5ccccc5)(c5ccccc5)c5ccccc5-6)c4)cc3)cc(-c3ccc(-c4ccccn4)cc3)n2)cc1]

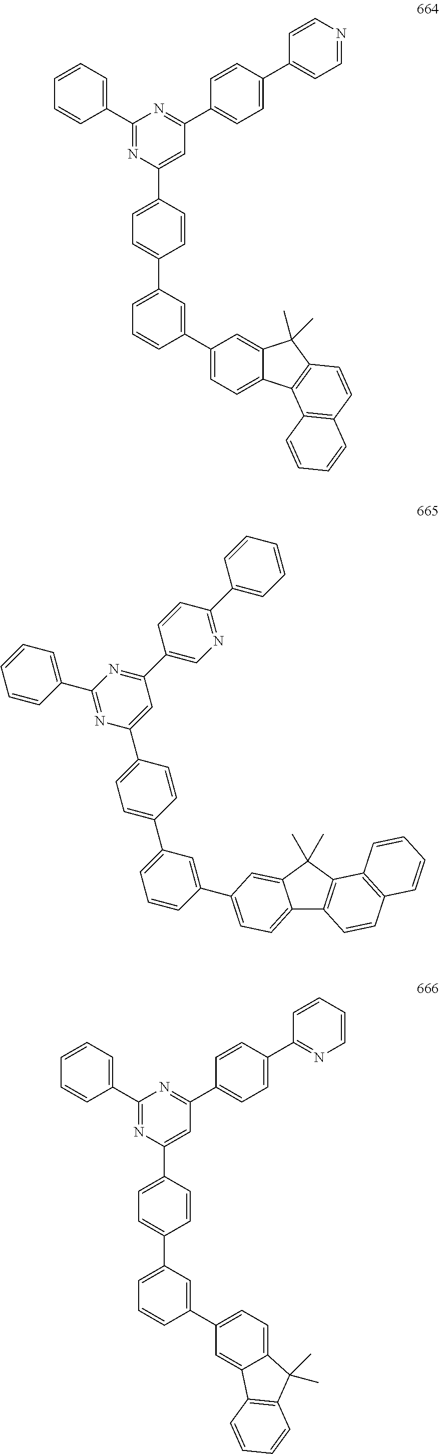 [SMILES: CC1(C)c2cc(-c3cccc(-c4ccc(-c5cc(-c6ccc(-c7ccncc7)cc6)nc(-c6ccccc6)n5)cc4)c3)ccc2-c2c1ccc1ccccc21 ; CC1(C)c2cc(-c3cccc(-c4ccc(-c5cc(-c6ccc(-c7ccccc7)nc6)nc(-c6ccccc6)n5)cc4)c3)ccc2-c2ccc3ccccc3c21 ; CC1(C)c2ccccc2-c2cc(-c3cccc(-c4ccc(-c5cc(-c6ccc(-c7ccccn7)cc6)nc(-c6ccccc6)n5)cc4)c3)ccc21]

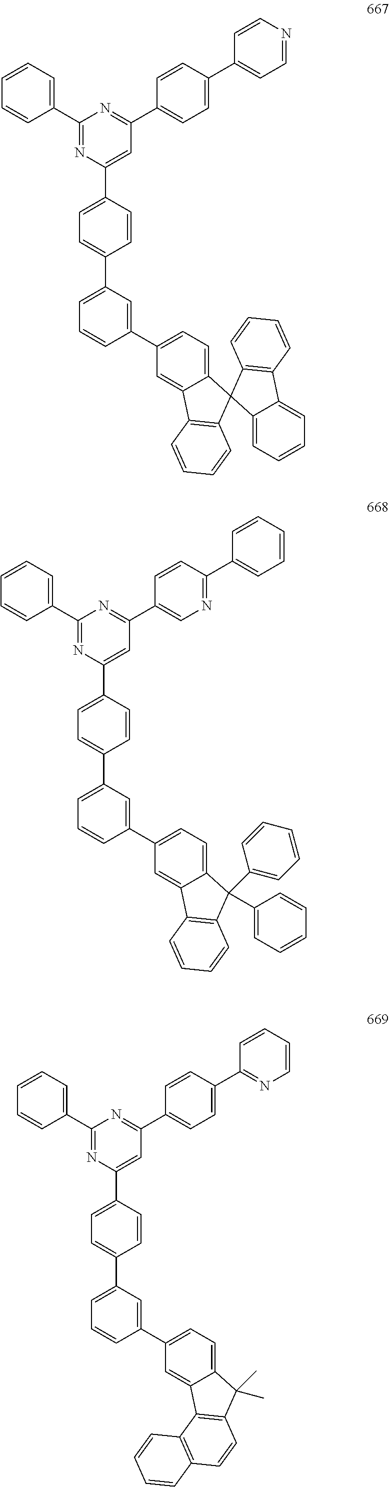 [SMILES: c1ccc(-c2nc(-c3ccc(-c4ccncc4)cc3)cc(-c3ccc(-c4cccc(-c5ccc6c(c5)-c5ccccc5C65c6ccccc6-c6ccccc65)c4)cc3)n2)cc1 ; c1ccc(-c2ccc(-c3cc(-c4ccc(-c5cccc(-c6ccc7c(c6)-c6ccccc6C7(c6ccccc6)c6ccccc6)c5)cc4)nc(-c4ccccc4)n3)cn2)cc1 ; CC1(C)c2ccc(-c3cccc(-c4ccc(-c5cc(-c6ccc(-c7ccccn7)cc6)nc(-c6ccccc6)n5)cc4)c3)cc2-c2c1ccc1ccccc21]

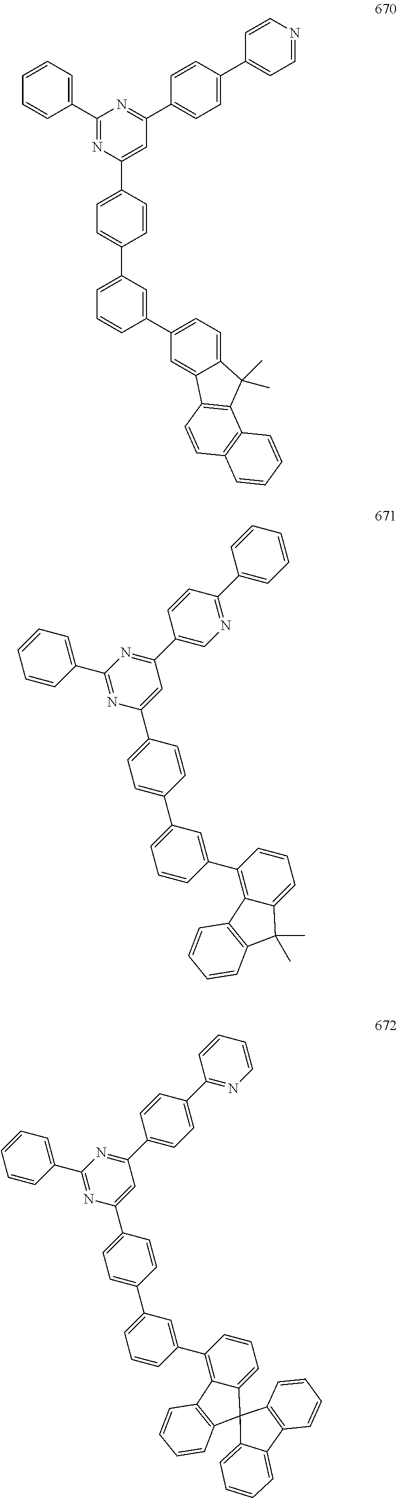 [SMILES: CC1(C)c2ccc(-c3cccc(-c4ccc(-c5cc(-c6ccc(-c7ccncc7)cc6)nc(-c6ccccc6)n5)cc4)c3)cc2-c2ccc3ccccc3c21 ; CC1(C)c2ccccc2-c2c(-c3cccc(-c4ccc(-c5cc(-c6ccc(-c7ccccc7)nc6)nc(-c6ccccc6)n5)cc4)c3)cccc21 ; c1ccc(-c2nc(-c3ccc(-c4cccc(-c5cccc6c5-c5ccccc5C65c6ccccc6-c6ccccc65)c4)cc3)cc(-c3ccc(-c4ccccn4)cc3)n2)cc1]

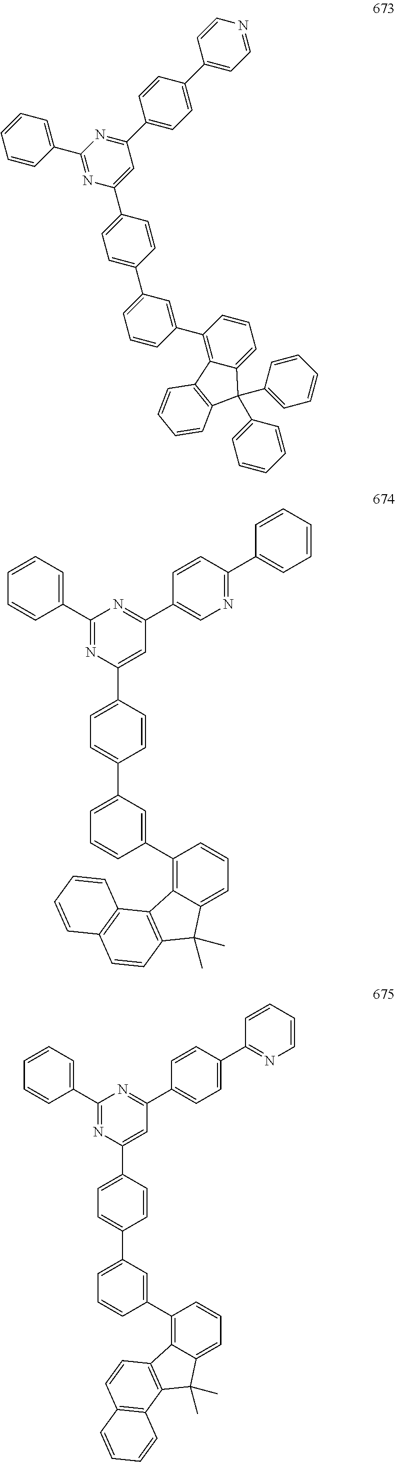 [SMILES: c1ccc(-c2nc(-c3ccc(-c4ccncc4)cc3)cc(-c3ccc(-c4cccc(-c5cccc6c5-c5ccccc5C6(c5ccccc5)c5ccccc5)c4)cc3)n2)cc1 ; CC1(C)c2cccc(-c3cccc(-c4ccc(-c5cc(-c6ccc(-c7ccccc7)nc6)nc(-c6ccccc6)n5)cc4)c3)c2-c2c1ccc1ccccc21 ; CC1(C)c2cccc(-c3cccc(-c4ccc(-c5cc(-c6ccc(-c7ccccn7)cc6)nc(-c6ccccc6)n5)cc4)c3)c2-c2ccc3ccccc3c21]

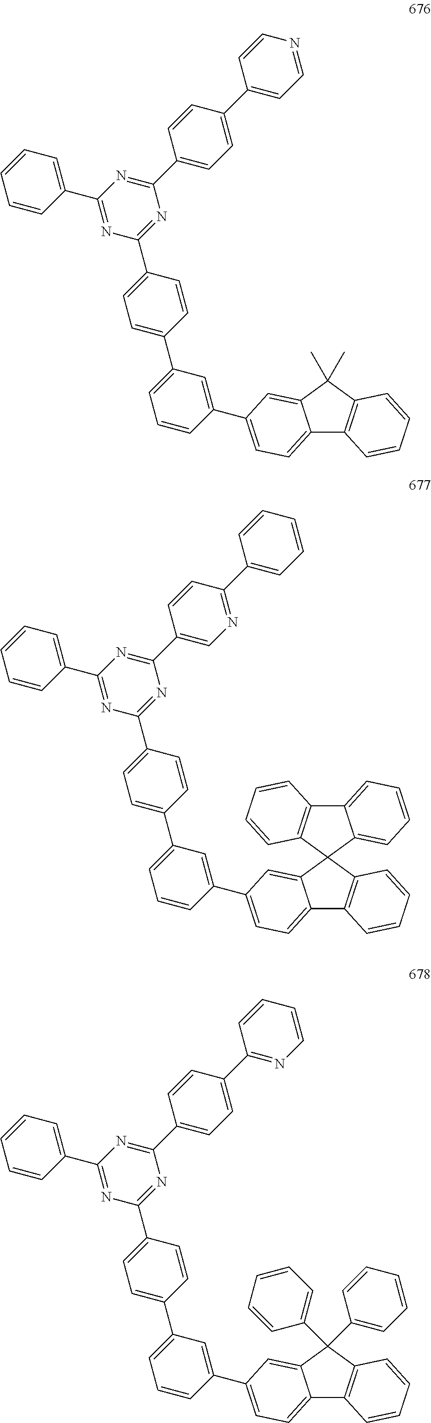 [SMILES: CC1(C)c2ccccc2-c2ccc(-c3cccc(-c4ccc(-c5nc(-c6ccccc6)nc(-c6ccc(-c7ccncc7)cc6)n5)cc4)c3)cc21 ; c1ccc(-c2ccc(-c3nc(-c4ccccc4)nc(-c4ccc(-c5cccc(-c6ccc7c(c6)C6(c8ccccc8-c8ccccc86)c6ccccc6-7)c5)cc4)n3)cn2)cc1 ; c1ccc(-c2nc(-c3ccc(-c4cccc(-c5ccc6c(c5)C(c5ccccc5)(c5ccccc5)c5ccccc5-6)c4)cc3)nc(-c3ccc(-c4ccccn4)cc3)n2)cc1]

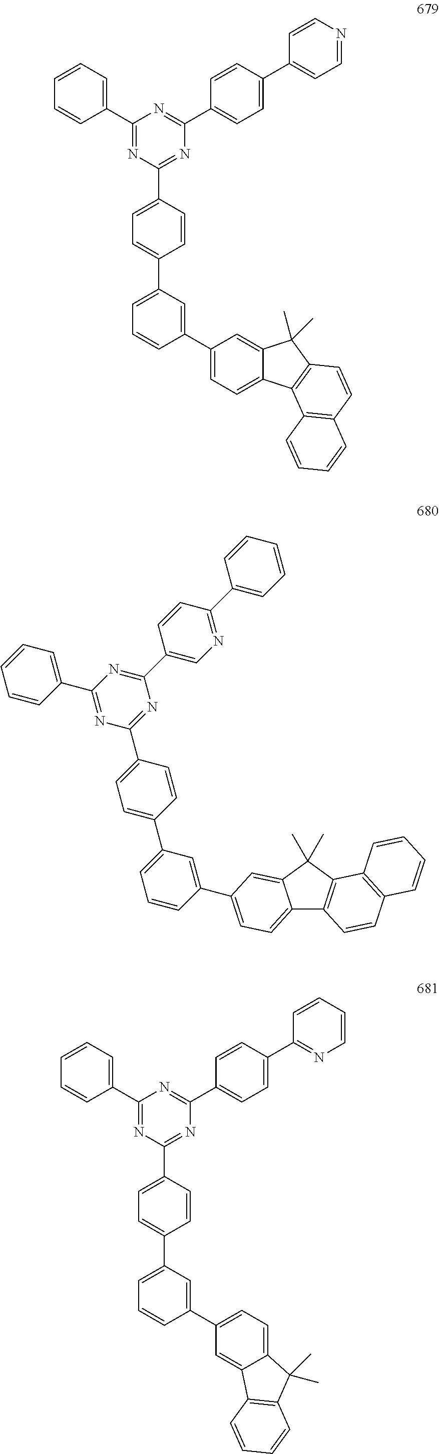 [SMILES: CC1(C)c2cc(-c3cccc(-c4ccc(-c5nc(-c6ccccc6)nc(-c6ccc(-c7ccncc7)cc6)n5)cc4)c3)ccc2-c2c1ccc1ccccc21 ; CC1(C)c2cc(-c3cccc(-c4ccc(-c5nc(-c6ccccc6)nc(-c6ccc(-c7ccccc7)nc6)n5)cc4)c3)ccc2-c2ccc3ccccc3c21 ; CC1(C)c2ccccc2-c2cc(-c3cccc(-c4ccc(-c5nc(-c6ccccc6)nc(-c6ccc(-c7ccccn7)cc6)n5)cc4)c3)ccc21]

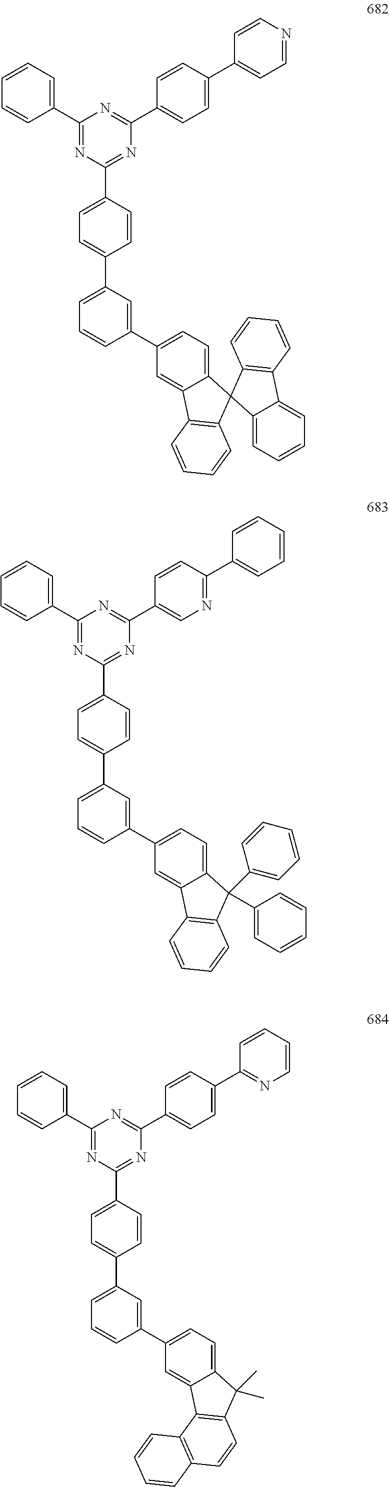 [SMILES: c1ccc(-c2nc(-c3ccc(-c4ccncc4)cc3)nc(-c3ccc(-c4cccc(-c5ccc6c(c5)-c5ccccc5C65c6ccccc6-c6ccccc65)c4)cc3)n2)cc1 ; c1ccc(-c2ccc(-c3nc(-c4ccccc4)nc(-c4ccc(-c5cccc(-c6ccc7c(c6)-c6ccccc6C7(c6ccccc6)c6ccccc6)c5)cc4)n3)cn2)cc1 ; CC1(C)c2ccc(-c3cccc(-c4ccc(-c5nc(-c6ccccc6)nc(-c6ccc(-c7ccccn7)cc6)n5)cc4)c3)cc2-c2c1ccc1ccccc21]

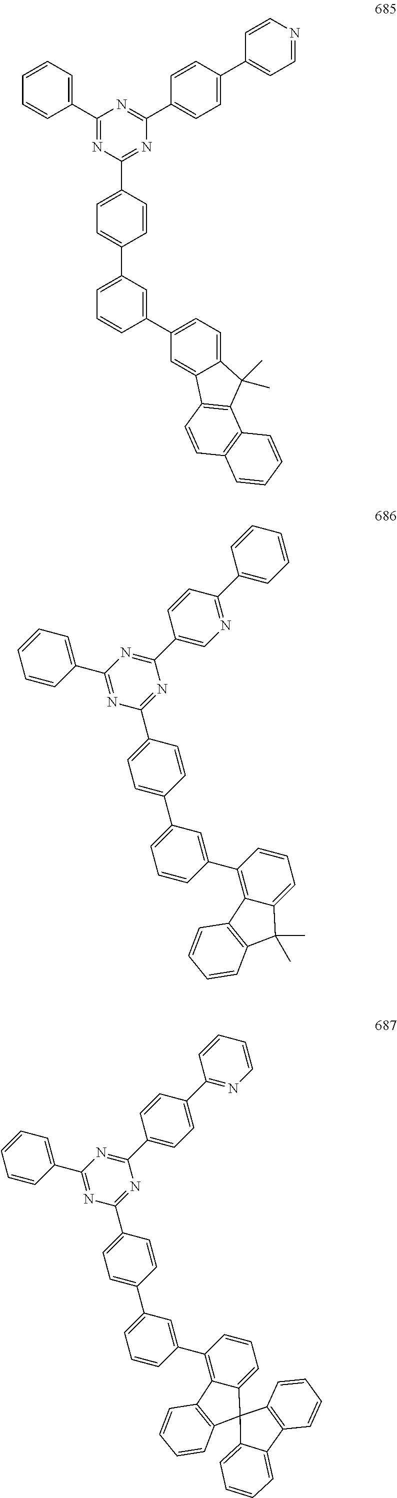 [SMILES: CC1(C)c2ccc(-c3cccc(-c4ccc(-c5nc(-c6ccccc6)nc(-c6ccc(-c7ccncc7)cc6)n5)cc4)c3)cc2-c2ccc3ccccc3c21 ; CC1(C)c2ccccc2-c2c(-c3cccc(-c4ccc(-c5nc(-c6ccccc6)nc(-c6ccc(-c7ccccc7)nc6)n5)cc4)c3)cccc21 ; c1ccc(-c2nc(-c3ccc(-c4cccc(-c5cccc6c5-c5ccccc5C65c6ccccc6-c6ccccc65)c4)cc3)nc(-c3ccc(-c4ccccn4)cc3)n2)cc1]

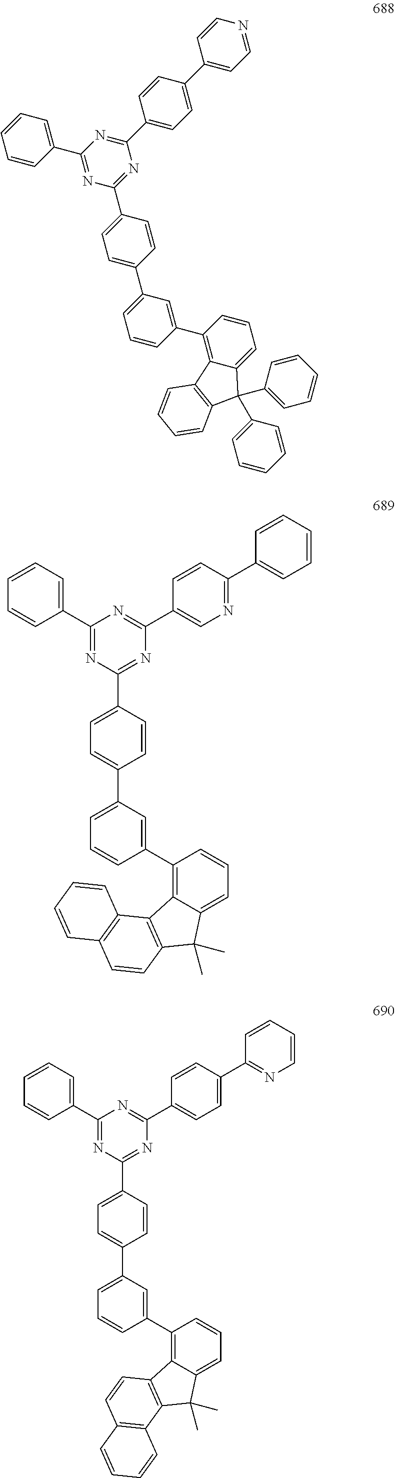 [SMILES: c1ccc(-c2nc(-c3ccc(-c4ccncc4)cc3)nc(-c3ccc(-c4cccc(-c5cccc6c5-c5ccccc5C6(c5ccccc5)c5ccccc5)c4)cc3)n2)cc1 ; CC1(C)c2cccc(-c3cccc(-c4ccc(-c5nc(-c6ccccc6)nc(-c6ccc(-c7ccccc7)nc6)n5)cc4)c3)c2-c2c1ccc1ccccc21 ; CC1(C)c2cccc(-c3cccc(-c4ccc(-c5nc(-c6ccccc6)nc(-c6ccc(-c7ccccn7)cc6)n5)cc4)c3)c2-c2ccc3ccccc3c21]

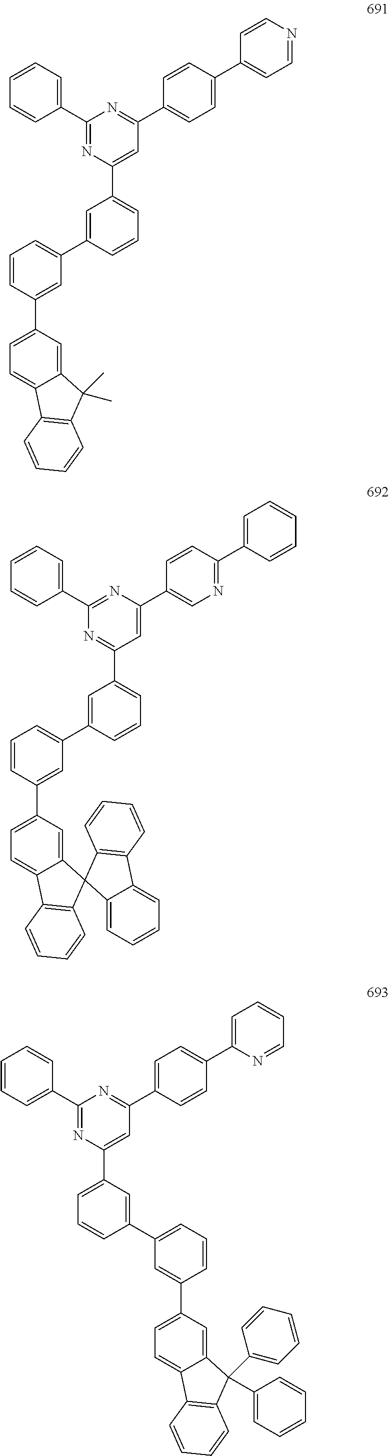 [SMILES: CC1(C)c2ccccc2-c2ccc(-c3cccc(-c4cccc(-c5cc(-c6ccc(-c7ccncc7)cc6)nc(-c6ccccc6)n5)c4)c3)cc21 ; c1ccc(-c2ccc(-c3cc(-c4cccc(-c5cccc(-c6ccc7c(c6)C6(c8ccccc8-c8ccccc86)c6ccccc6-7)c5)c4)nc(-c4ccccc4)n3)cn2)cc1 ; c1ccc(-c2nc(-c3ccc(-c4ccccn4)cc3)cc(-c3cccc(-c4cccc(-c5ccc6c(c5)C(c5ccccc5)(c5ccccc5)c5ccccc5-6)c4)c3)n2)cc1]

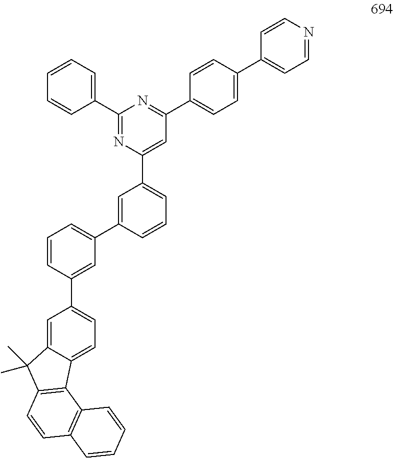 [SMILES: CC1(C)c2cc(-c3cccc(-c4cccc(-c5cc(-c6ccc(-c7ccncc7)cc6)nc(-c6ccccc6)n5)c4)c3)ccc2-c2c1ccc1ccccc21]

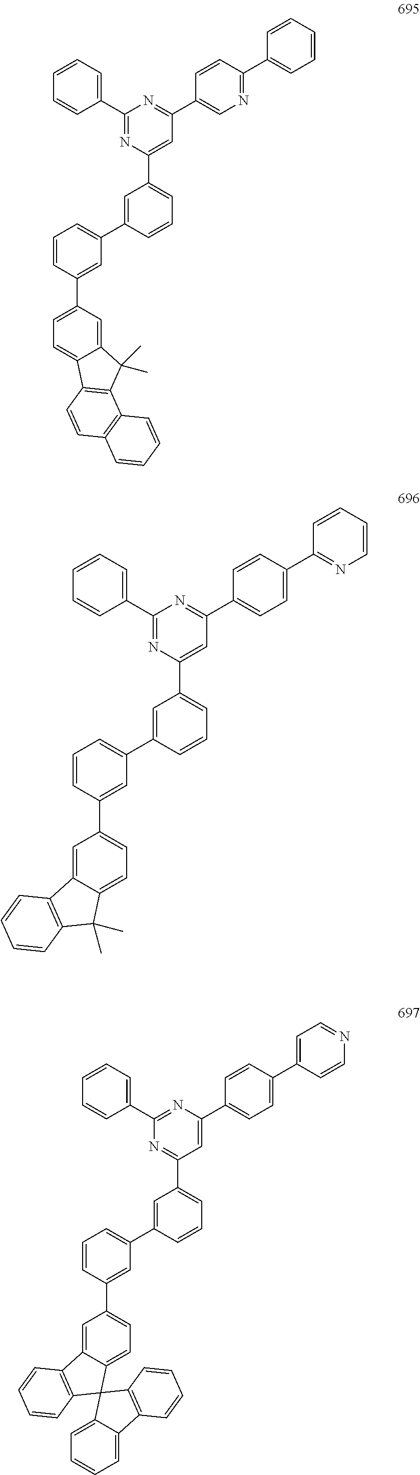 [SMILES: CC1(C)c2cc(-c3cccc(-c4cccc(-c5cc(-c6ccc(-c7ccccc7)nc6)nc(-c6ccccc6)n5)c4)c3)ccc2-c2ccc3ccccc3c21 ; CC1(C)c2ccccc2-c2cc(-c3cccc(-c4cccc(-c5cc(-c6ccc(-c7ccccn7)cc6)nc(-c6ccccc6)n5)c4)c3)ccc21 ; c1ccc(-c2nc(-c3ccc(-c4ccncc4)cc3)cc(-c3cccc(-c4cccc(-c5ccc6c(c5)-c5ccccc5C65c6ccccc6-c6ccccc65)c4)c3)n2)cc1]

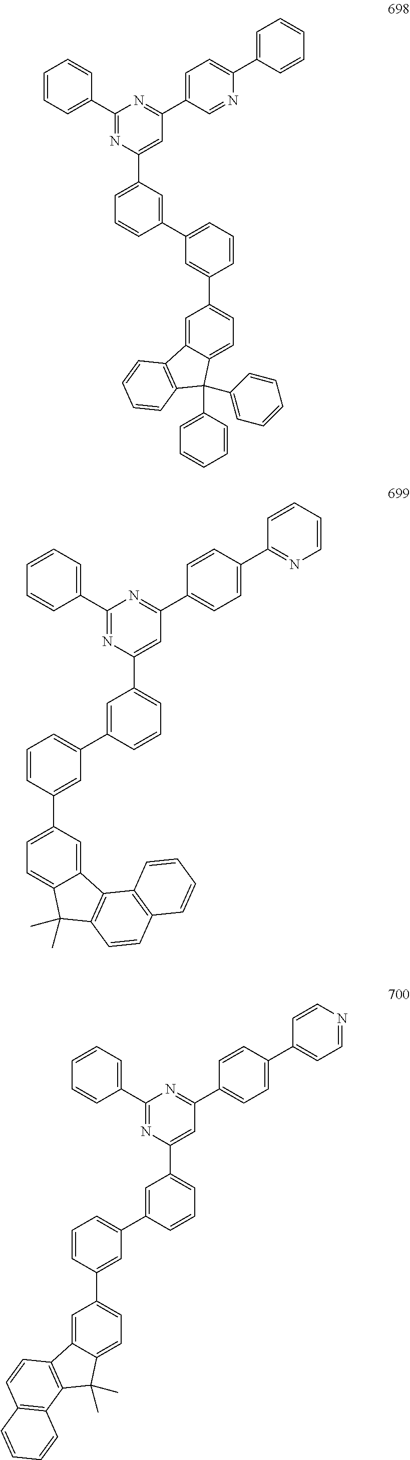 [SMILES: c1ccc(-c2ccc(-c3cc(-c4cccc(-c5cccc(-c6ccc7c(c6)-c6ccccc6C7(c6ccccc6)c6ccccc6)c5)c4)nc(-c4ccccc4)n3)cn2)cc1 ; CC1(C)c2ccc(-c3cccc(-c4cccc(-c5cc(-c6ccc(-c7ccccn7)cc6)nc(-c6ccccc6)n5)c4)c3)cc2-c2c1ccc1ccccc21 ; CC1(C)c2ccc(-c3cccc(-c4cccc(-c5cc(-c6ccc(-c7ccncc7)cc6)nc(-c6ccccc6)n5)c4)c3)cc2-c2ccc3ccccc3c21]

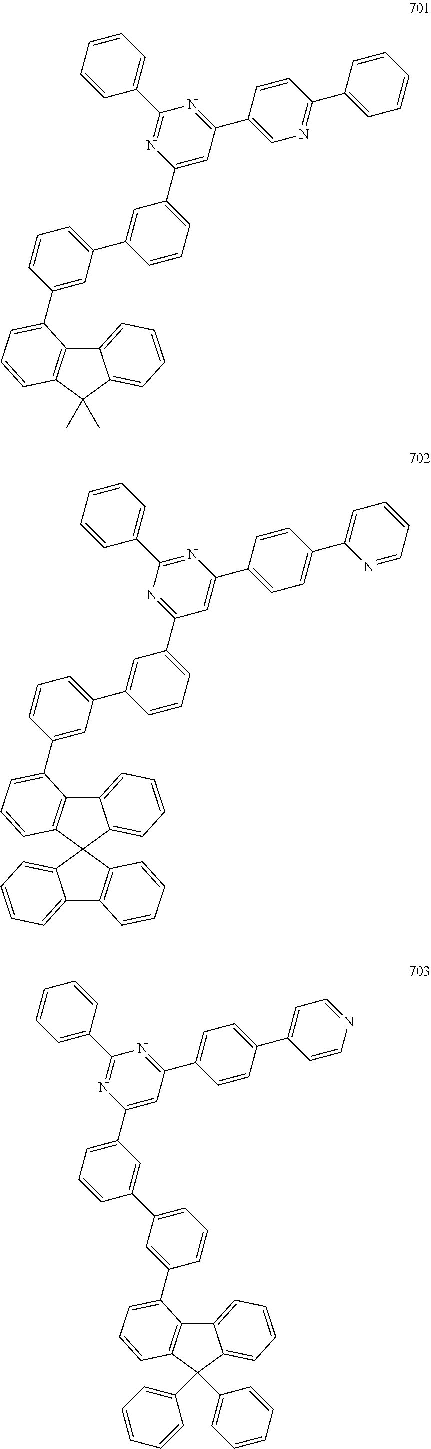 [SMILES: CC1(C)c2ccccc2-c2c(-c3cccc(-c4cccc(-c5cc(-c6ccc(-c7ccccc7)nc6)nc(-c6ccccc6)n5)c4)c3)cccc21 ; c1ccc(-c2nc(-c3ccc(-c4ccccn4)cc3)cc(-c3cccc(-c4cccc(-c5cccc6c5-c5ccccc5C65c6ccccc6-c6ccccc65)c4)c3)n2)cc1 ; c1ccc(-c2nc(-c3ccc(-c4ccncc4)cc3)cc(-c3cccc(-c4cccc(-c5cccc6c5-c5ccccc5C6(c5ccccc5)c5ccccc5)c4)c3)n2)cc1]

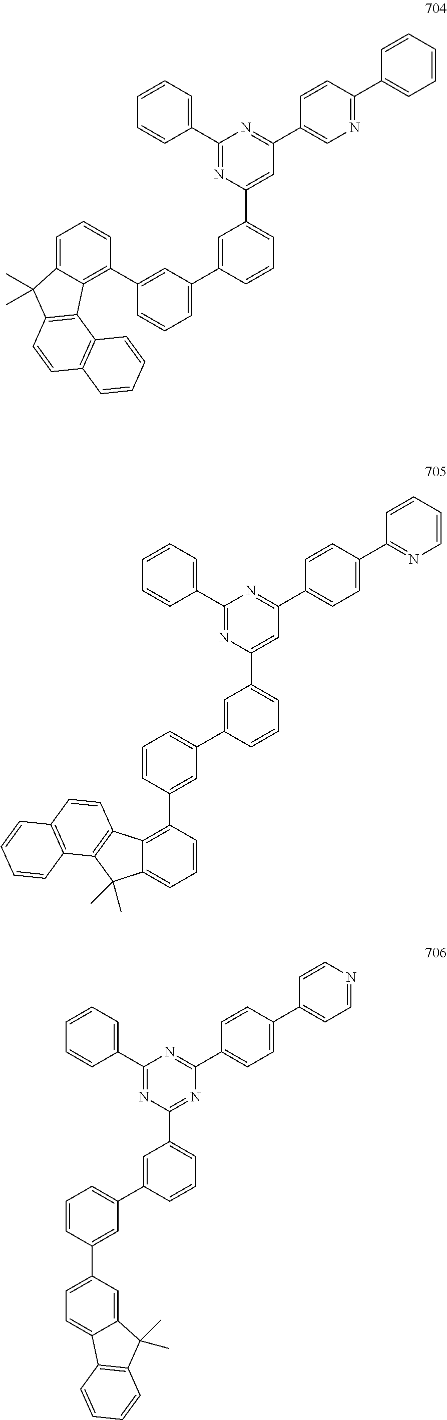 [SMILES: CC1(C)c2cccc(-c3cccc(-c4cccc(-c5cc(-c6ccc(-c7ccccc7)nc6)nc(-c6ccccc6)n5)c4)c3)c2-c2c1ccc1ccccc21 ; CC1(C)c2cccc(-c3cccc(-c4cccc(-c5cc(-c6ccc(-c7ccccn7)cc6)nc(-c6ccccc6)n5)c4)c3)c2-c2ccc3ccccc3c21 ; CC1(C)c2ccccc2-c2ccc(-c3cccc(-c4cccc(-c5nc(-c6ccccc6)nc(-c6ccc(-c7ccncc7)cc6)n5)c4)c3)cc21]

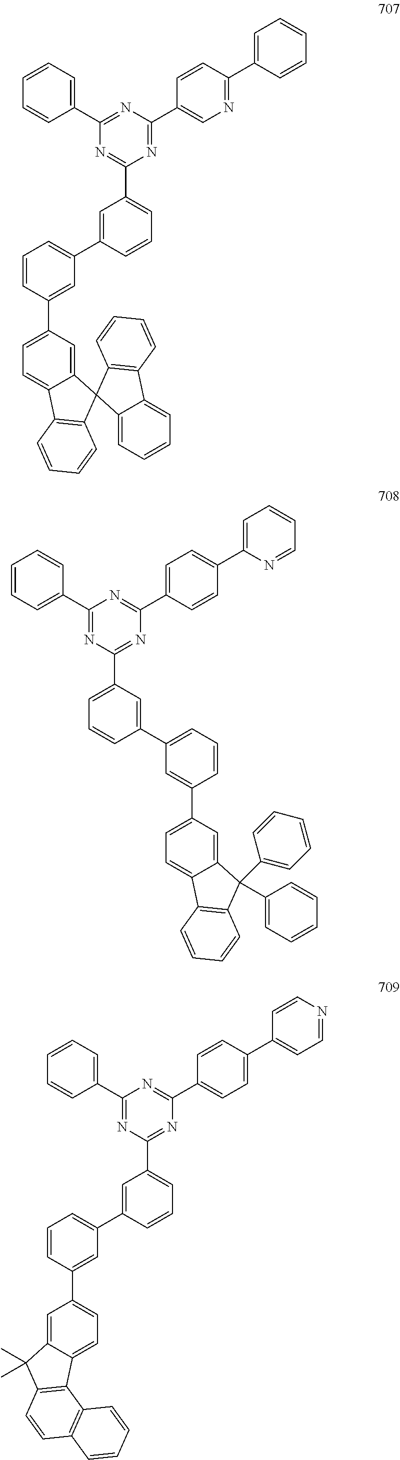 [SMILES: c1ccc(-c2ccc(-c3nc(-c4ccccc4)nc(-c4cccc(-c5cccc(-c6ccc7c(c6)C6(c8ccccc8-c8ccccc86)c6ccccc6-7)c5)c4)n3)cn2)cc1 ; c1ccc(-c2nc(-c3ccc(-c4ccccn4)cc3)nc(-c3cccc(-c4cccc(-c5ccc6c(c5)C(c5ccccc5)(c5ccccc5)c5ccccc5-6)c4)c3)n2)cc1 ; CC1(C)c2cc(-c3cccc(-c4cccc(-c5nc(-c6ccccc6)nc(-c6ccc(-c7ccncc7)cc6)n5)c4)c3)ccc2-c2c1ccc1ccccc21]

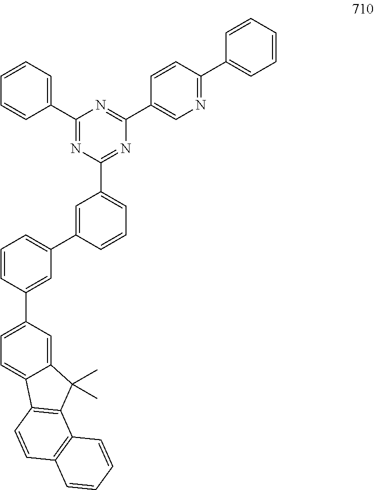 [SMILES: CC1(C)c2cc(-c3cccc(-c4cccc(-c5nc(-c6ccccc6)nc(-c6ccc(-c7ccccc7)nc6)n5)c4)c3)ccc2-c2ccc3ccccc3c21]

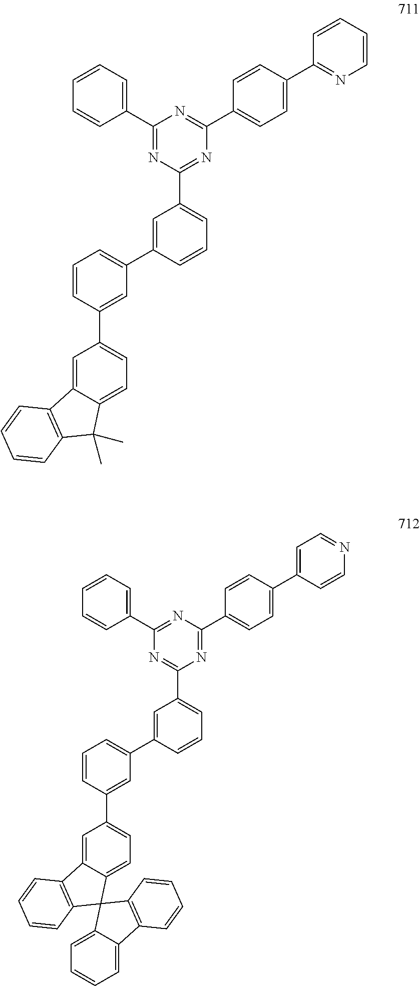 [SMILES: CC1(C)c2ccccc2-c2cc(-c3cccc(-c4cccc(-c5nc(-c6ccccc6)nc(-c6ccc(-c7ccccn7)cc6)n5)c4)c3)ccc21 ; c1ccc(-c2nc(-c3ccc(-c4ccncc4)cc3)nc(-c3cccc(-c4cccc(-c5ccc6c(c5)-c5ccccc5C65c6ccccc6-c6ccccc65)c4)c3)n2)cc1]

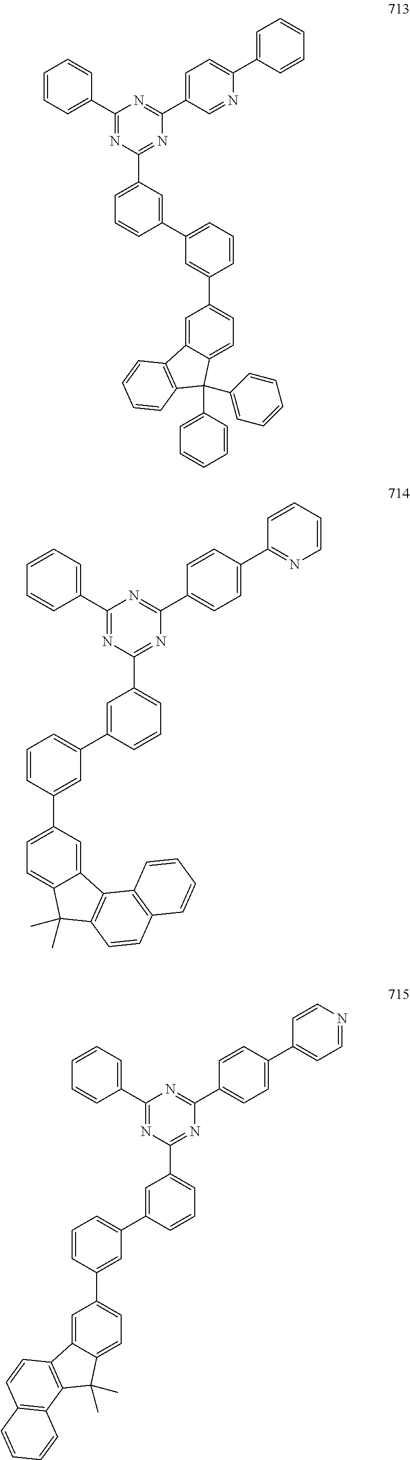 [SMILES: c1ccc(-c2ccc(-c3nc(-c4ccccc4)nc(-c4cccc(-c5cccc(-c6ccc7c(c6)-c6ccccc6C7(c6ccccc6)c6ccccc6)c5)c4)n3)cn2)cc1 ; CC1(C)c2ccc(-c3cccc(-c4cccc(-c5nc(-c6ccccc6)nc(-c6ccc(-c7ccccn7)cc6)n5)c4)c3)cc2-c2c1ccc1ccccc21 ; CC1(C)c2ccc(-c3cccc(-c4cccc(-c5nc(-c6ccccc6)nc(-c6ccc(-c7ccncc7)cc6)n5)c4)c3)cc2-c2ccc3ccccc3c21]

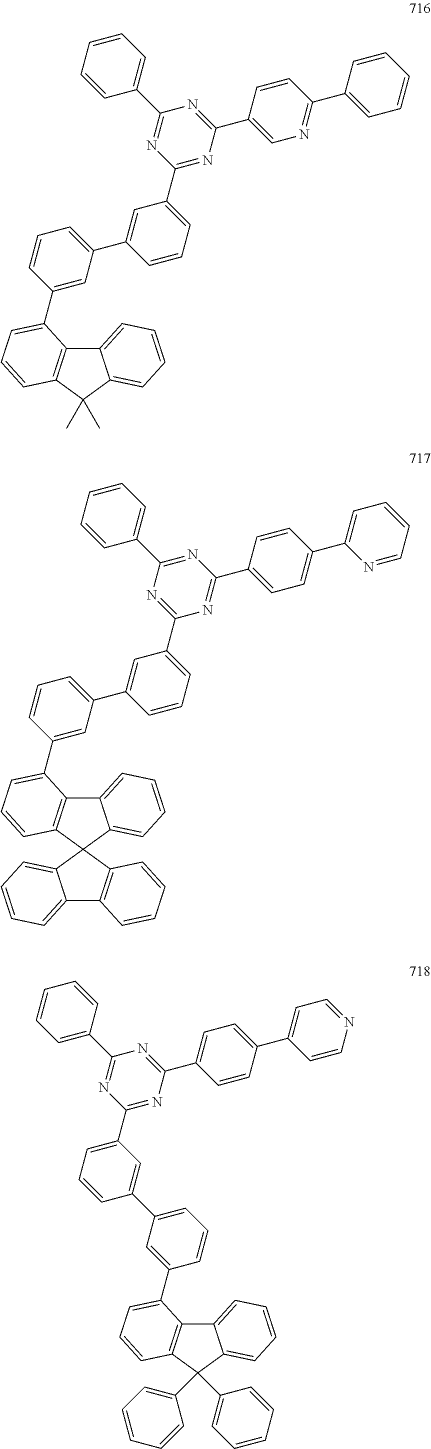 [SMILES: CC1(C)c2ccccc2-c2c(-c3cccc(-c4cccc(-c5nc(-c6ccccc6)nc(-c6ccc(-c7ccccc7)nc6)n5)c4)c3)cccc21 ; c1ccc(-c2nc(-c3ccc(-c4ccccn4)cc3)nc(-c3cccc(-c4cccc(-c5cccc6c5-c5ccccc5C65c6ccccc6-c6ccccc65)c4)c3)n2)cc1 ; c1ccc(-c2nc(-c3ccc(-c4ccncc4)cc3)nc(-c3cccc(-c4cccc(-c5cccc6c5-c5ccccc5C6(c5ccccc5)c5ccccc5)c4)c3)n2)cc1]

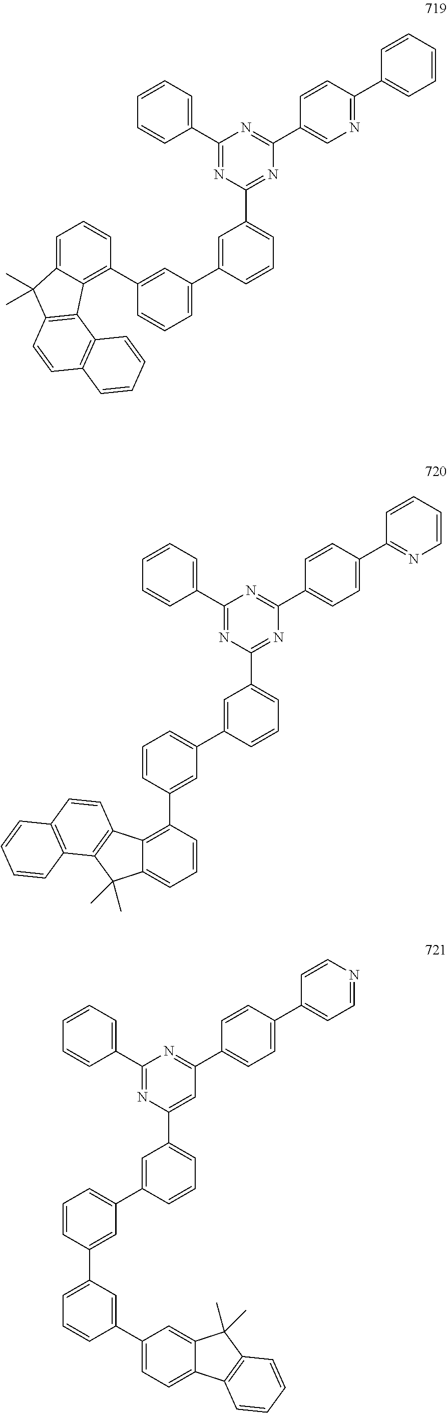 [SMILES: CC1(C)c2cccc(-c3cccc(-c4cccc(-c5nc(-c6ccccc6)nc(-c6ccc(-c7ccccc7)nc6)n5)c4)c3)c2-c2c1ccc1ccccc21 ; CC1(C)c2cccc(-c3cccc(-c4cccc(-c5nc(-c6ccccc6)nc(-c6ccc(-c7ccccn7)cc6)n5)c4)c3)c2-c2ccc3ccccc3c21 ; CC1(C)c2ccccc2-c2ccc(-c3cccc(-c4cccc(-c5cccc(-c6cc(-c7ccc(-c8ccncc8)cc7)nc(-c7ccccc7)n6)c5)c4)c3)cc21]

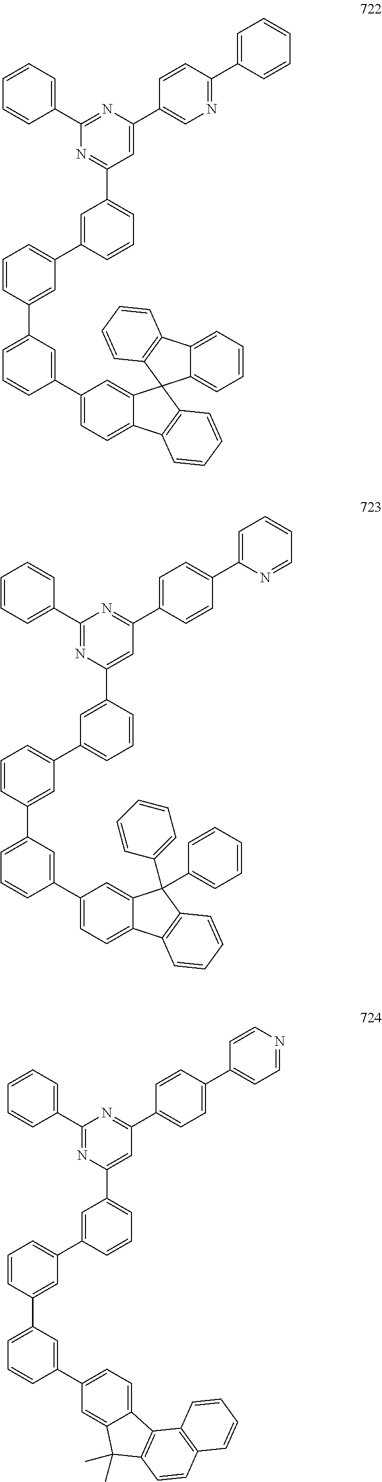 [SMILES: c1ccc(-c2ccc(-c3cc(-c4cccc(-c5cccc(-c6cccc(-c7ccc8c(c7)C7(c9ccccc9-c9ccccc97)c7ccccc7-8)c6)c5)c4)nc(-c4ccccc4)n3)cn2)cc1 ; c1ccc(-c2nc(-c3ccc(-c4ccccn4)cc3)cc(-c3cccc(-c4cccc(-c5cccc(-c6ccc7c(c6)C(c6ccccc6)(c6ccccc6)c6ccccc6-7)c5)c4)c3)n2)cc1 ; CC1(C)c2cc(-c3cccc(-c4cccc(-c5cccc(-c6cc(-c7ccc(-c8ccncc8)cc7)nc(-c7ccccc7)n6)c5)c4)c3)ccc2-c2c1ccc1ccccc21]

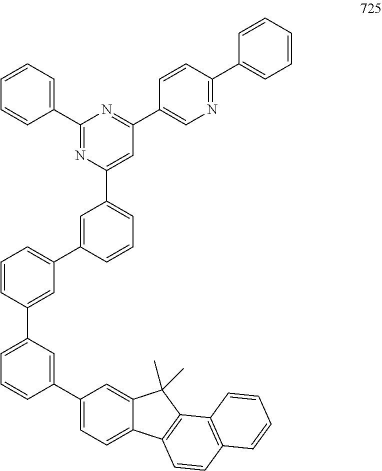 [SMILES: CC1(C)c2cc(-c3cccc(-c4cccc(-c5cccc(-c6cc(-c7ccc(-c8ccccc8)nc7)nc(-c7ccccc7)n6)c5)c4)c3)ccc2-c2ccc3ccccc3c21]

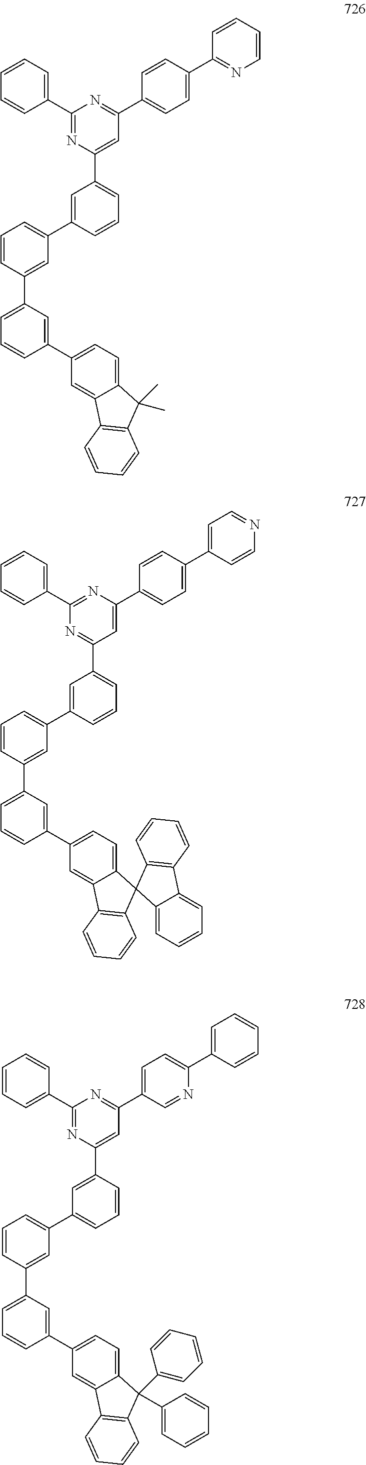 [SMILES: CC1(C)c2ccccc2-c2cc(-c3cccc(-c4cccc(-c5cccc(-c6cc(-c7ccc(-c8ccccn8)cc7)nc(-c7ccccc7)n6)c5)c4)c3)ccc21 ; c1ccc(-c2nc(-c3ccc(-c4ccncc4)cc3)cc(-c3cccc(-c4cccc(-c5cccc(-c6ccc7c(c6)-c6ccccc6C76c7ccccc7-c7ccccc76)c5)c4)c3)n2)cc1 ; c1ccc(-c2ccc(-c3cc(-c4cccc(-c5cccc(-c6cccc(-c7ccc8c(c7)-c7ccccc7C8(c7ccccc7)c7ccccc7)c6)c5)c4)nc(-c4ccccc4)n3)cn2)cc1]

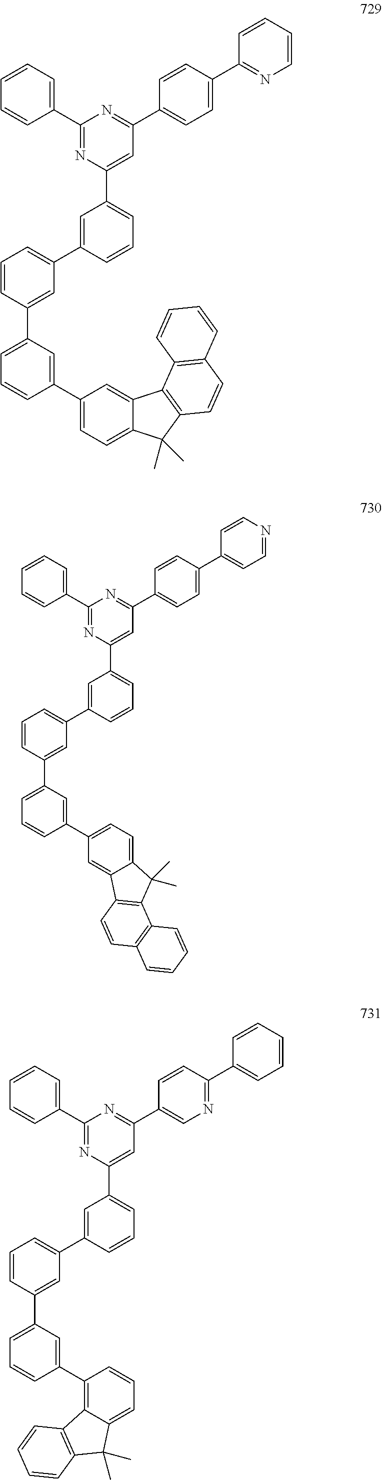 [SMILES: CC1(C)c2ccc(-c3cccc(-c4cccc(-c5cccc(-c6cc(-c7ccc(-c8ccccn8)cc7)nc(-c7ccccc7)n6)c5)c4)c3)cc2-c2c1ccc1ccccc21 ; CC1(C)c2ccc(-c3cccc(-c4cccc(-c5cccc(-c6cc(-c7ccc(-c8ccncc8)cc7)nc(-c7ccccc7)n6)c5)c4)c3)cc2-c2ccc3ccccc3c21 ; CC1(C)c2ccccc2-c2c(-c3cccc(-c4cccc(-c5cccc(-c6cc(-c7ccc(-c8ccccc8)nc7)nc(-c7ccccc7)n6)c5)c4)c3)cccc21]

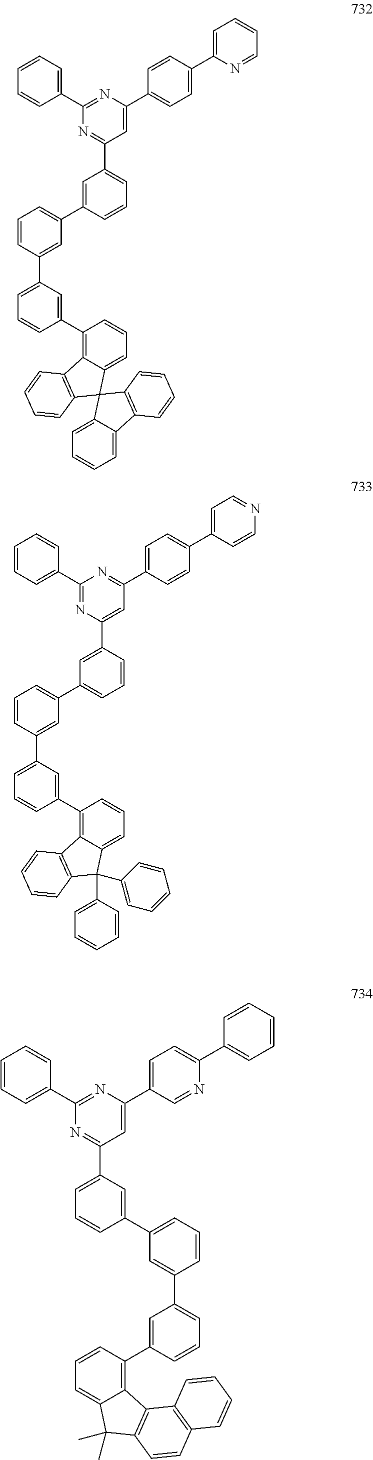 [SMILES: c1ccc(-c2nc(-c3ccc(-c4ccccn4)cc3)cc(-c3cccc(-c4cccc(-c5cccc(-c6cccc7c6-c6ccccc6C76c7ccccc7-c7ccccc76)c5)c4)c3)n2)cc1 ; c1ccc(-c2nc(-c3ccc(-c4ccncc4)cc3)cc(-c3cccc(-c4cccc(-c5cccc(-c6cccc7c6-c6ccccc6C7(c6ccccc6)c6ccccc6)c5)c4)c3)n2)cc1 ; CC1(C)c2cccc(-c3cccc(-c4cccc(-c5cccc(-c6cc(-c7ccc(-c8ccccc8)nc7)nc(-c7ccccc7)n6)c5)c4)c3)c2-c2c1ccc1ccccc21]

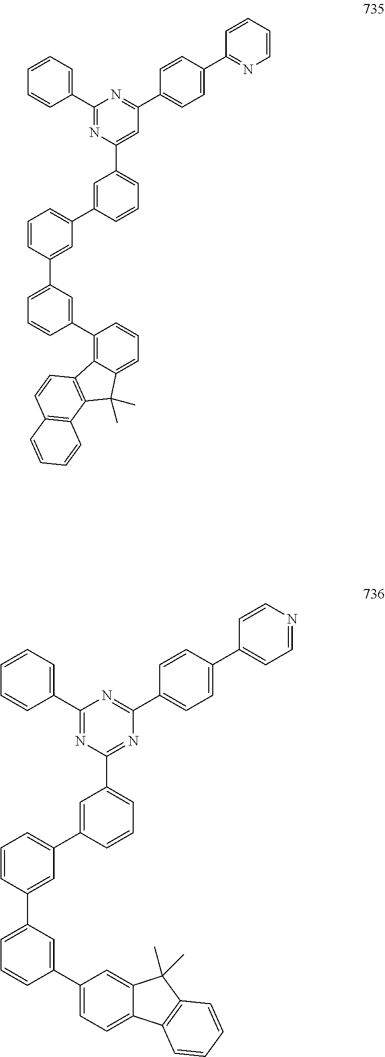 [SMILES: CC1(C)c2cccc(-c3cccc(-c4cccc(-c5cccc(-c6cc(-c7ccc(-c8ccccn8)cc7)nc(-c7ccccc7)n6)c5)c4)c3)c2-c2ccc3ccccc3c21 ; CC1(C)c2ccccc2-c2ccc(-c3cccc(-c4cccc(-c5cccc(-c6nc(-c7ccccc7)nc(-c7ccc(-c8ccncc8)cc7)n6)c5)c4)c3)cc21]

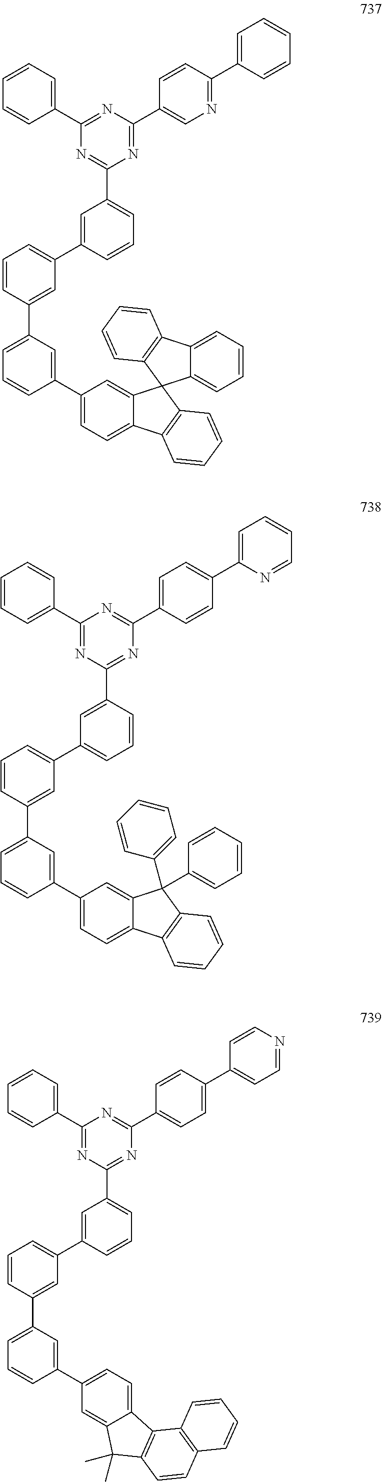 [SMILES: c1ccc(-c2ccc(-c3nc(-c4ccccc4)nc(-c4cccc(-c5cccc(-c6cccc(-c7ccc8c(c7)C7(c9ccccc9-c9ccccc97)c7ccccc7-8)c6)c5)c4)n3)cn2)cc1 ; c1ccc(-c2nc(-c3ccc(-c4ccccn4)cc3)nc(-c3cccc(-c4cccc(-c5cccc(-c6ccc7c(c6)C(c6ccccc6)(c6ccccc6)c6ccccc6-7)c5)c4)c3)n2)cc1 ; CC1(C)c2cc(-c3cccc(-c4cccc(-c5cccc(-c6nc(-c7ccccc7)nc(-c7ccc(-c8ccncc8)cc7)n6)c5)c4)c3)ccc2-c2c1ccc1ccccc21]

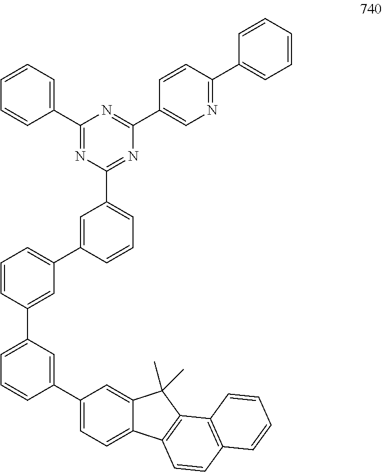 [SMILES: CC1(C)c2cc(-c3cccc(-c4cccc(-c5cccc(-c6nc(-c7ccccc7)nc(-c7ccc(-c8ccccc8)nc7)n6)c5)c4)c3)ccc2-c2ccc3ccccc3c21]

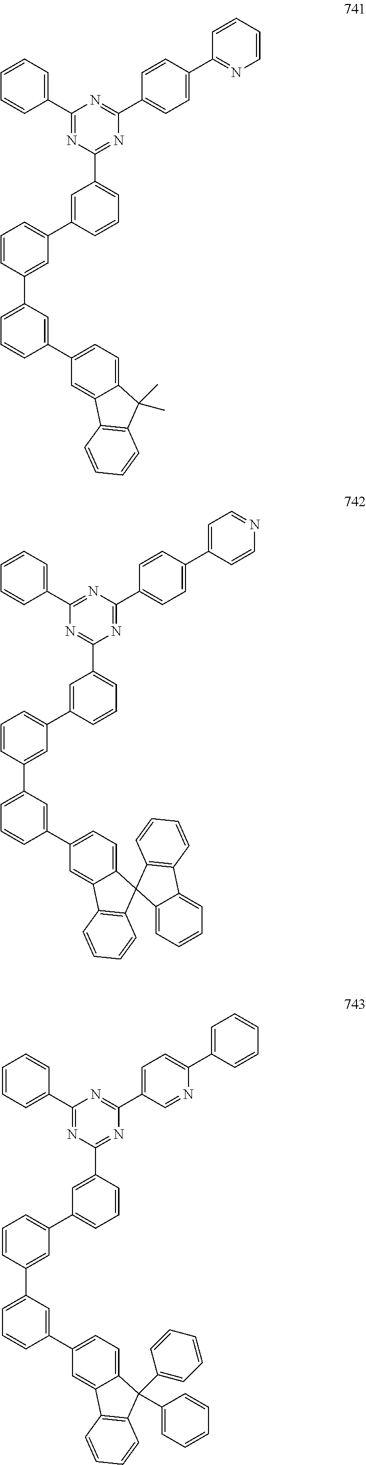 [SMILES: CC1(C)c2ccccc2-c2cc(-c3cccc(-c4cccc(-c5cccc(-c6nc(-c7ccccc7)nc(-c7ccc(-c8ccccn8)cc7)n6)c5)c4)c3)ccc21 ; c1ccc(-c2nc(-c3ccc(-c4ccncc4)cc3)nc(-c3cccc(-c4cccc(-c5cccc(-c6ccc7c(c6)-c6ccccc6C76c7ccccc7-c7ccccc76)c5)c4)c3)n2)cc1 ; c1ccc(-c2ccc(-c3nc(-c4ccccc4)nc(-c4cccc(-c5cccc(-c6cccc(-c7ccc8c(c7)-c7ccccc7C8(c7ccccc7)c7ccccc7)c6)c5)c4)n3)cn2)cc1]

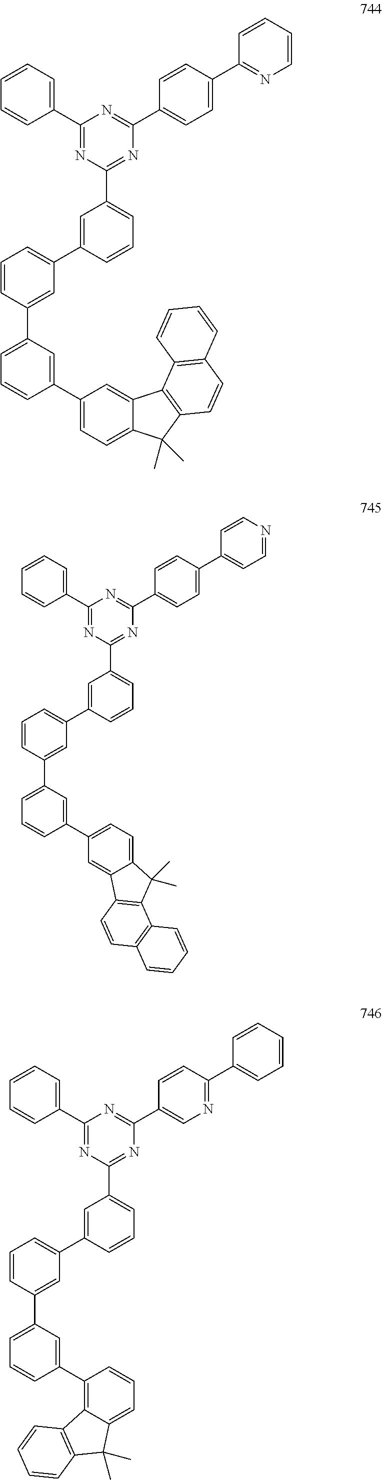 [SMILES: CC1(C)c2ccc(-c3cccc(-c4cccc(-c5cccc(-c6nc(-c7ccccc7)nc(-c7ccc(-c8ccccn8)cc7)n6)c5)c4)c3)cc2-c2c1ccc1ccccc21 ; CC1(C)c2ccc(-c3cccc(-c4cccc(-c5cccc(-c6nc(-c7ccccc7)nc(-c7ccc(-c8ccncc8)cc7)n6)c5)c4)c3)cc2-c2ccc3ccccc3c21 ; CC1(C)c2ccccc2-c2c(-c3cccc(-c4cccc(-c5cccc(-c6nc(-c7ccccc7)nc(-c7ccc(-c8ccccc8)nc7)n6)c5)c4)c3)cccc21]

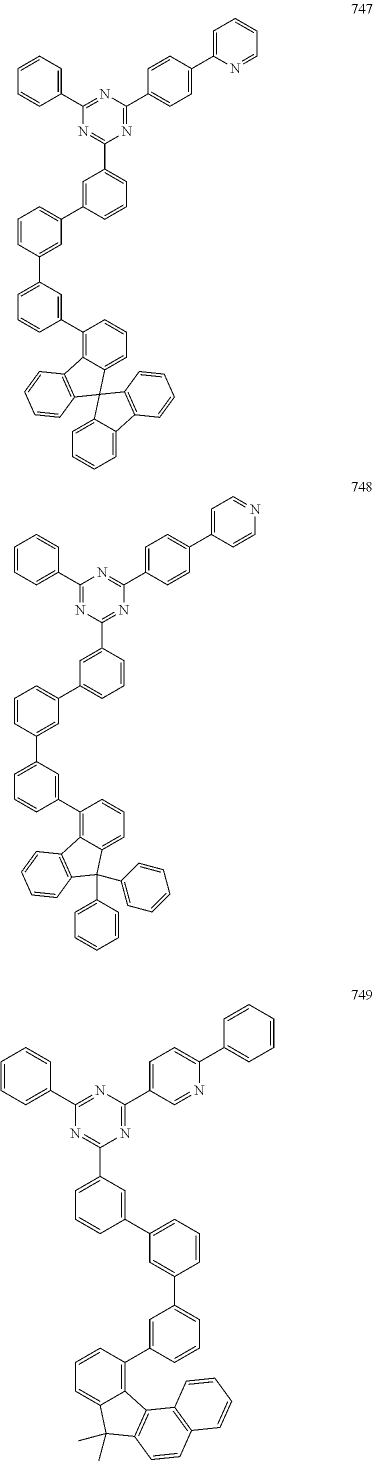 [SMILES: c1ccc(-c2nc(-c3ccc(-c4ccccn4)cc3)nc(-c3cccc(-c4cccc(-c5cccc(-c6cccc7c6-c6ccccc6C76c7ccccc7-c7ccccc76)c5)c4)c3)n2)cc1 ; c1ccc(-c2nc(-c3ccc(-c4ccncc4)cc3)nc(-c3cccc(-c4cccc(-c5cccc(-c6cccc7c6-c6ccccc6C7(c6ccccc6)c6ccccc6)c5)c4)c3)n2)cc1 ; CC1(C)c2cccc(-c3cccc(-c4cccc(-c5cccc(-c6nc(-c7ccccc7)nc(-c7ccc(-c8ccccc8)nc7)n6)c5)c4)c3)c2-c2c1ccc1ccccc21]

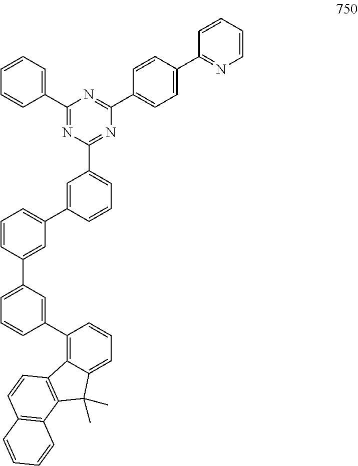 [SMILES: CC1(C)c2cccc(-c3cccc(-c4cccc(-c5cccc(-c6nc(-c7ccccc7)nc(-c7ccc(-c8ccccn8)cc7)n6)c5)c4)c3)c2-c2ccc3ccccc3c21]

As used herein, “alkyl” refers to a monovalent functional group obtained by removing a hydrogen atom from a saturated, linear or branched hydrocarbon having 1 to 40 carbon atoms. Examples of such alkyl may include, but are not limited to, methyl, ethyl, propyl, isobutyl, sec-butyl, pentyl, iso-amyl, hexyl or the like.

As used herein, “alkenyl” refers to a monovalent substituent derived from an unsaturated, linear or branched hydrocarbon having 2 to 40 carbon atoms, having at least one carbon-carbon double bond. Examples of such alkenyl may include, but are not limited to, vinyl, allyl, isopropenyl, 2-butenyl or the like.

As used herein, “alkynyl” refers to a monovalent substituent derived from an unsaturated, linear or branched hydrocarbon having 2 to 40 carbon atoms, having at least one carbon-carbon triple bond. Examples of such alkynyl may include, but are not limited to, ethynyl, 2-propynyl or the like.

As used herein, “aryl” refers to a monovalent substituent derived from an aromatic hydrocarbon having 6 to 60 carbon atoms, which is in a structure with a single ring or two or more rings combined with each other. In addition, a form in which two or more rings are pendant (e.g., simply attached) to or fused with each other may also be included. Examples of such aryl may include, but are not limited to, phenyl, naphthyl, phenanthryl, anthryl or the like.

As used herein, “heteroaryl” refers to a monovalent substituent derived from a monoheterocyclic or polyheterocyclic aromatic hydrocarbon having 5 to 60 nuclear atoms. In such a case, one or more carbons in the ring, preferably one to three carbons, are substituted with a heteroatom such as N, O, S or Se. In addition, a form in which two or more rings are pendant to or fused with each other may be included, and a form fused with an aryl group may also be included. Examples of such heteroaryl may include, but are not limited to, a 6-membered monocyclic ring such as pyridyl, pyrazinyl, pyrimidinyl, pyridazinyl and triazinyl; a polycyclic ring such as phenoxathienyl, indolizinyl, indolyl, purinyl, quinolyl, benzothiazole and carbazolyl; 2-furanyl; N-imidazolyl; 2-isoxazolyl; 2-pyridinyl; 2-pyrimidinyl or the like.

As used herein, “aryloxy” refers to a monovalent functional group represented by R″O—, where R″ is aryl having 6 to 60 carbon atoms. Examples of such aryloxy may include, but are not limited to, phenyloxy, naphthyloxy, diphenyloxy or the like.

As used herein, “alkyloxy” refers to a monovalent functional group represented by R′O—, where R′ is alkyl having 1 to 40 carbon atoms. Such alkyloxy may include a linear, branched or cyclic structure. Examples of such alkyloxy may include, but are not limited to, methoxy, ethoxy, n-propoxy, 1-propoxy, t-butoxy, n-butoxy, pentoxy or the like.

As used herein, “cycloalkyl” refers to a monovalent functional group obtained by removing a hydrogen atom from a monocyclic or polycyclic non-aromatic hydrocarbon (saturated cyclic hydrocarbon) having 3 to 40 carbon atoms. Examples of such cycloalkyl may include, but are not limited to, cyclopropyl, cyclopentyl, cyclohexyl, norbornyl, adamantine or the like.

As used herein, “heterocycloalkyl” refers to a monovalent functional group obtained by removing a hydrogen atom from a non-aromatic hydrocarbon (saturated cyclic hydrocarbon) having 3 to 40 nuclear atoms, where one or more carbons in the ring, preferably one to three carbons, are substituted with a heteroatom such as N, O, or S. Examples of such heterocycloalkyl may include, but are not limited to, morpholine, piperazine or the like.

As used herein, “alkylsilyl” refers to silyl substituted with alkyl having 1 to 40 carbon atoms, “arylsilyl” refers to silyl substituted with aryl having 6 to 60 carbon atoms, “alkylboron group” refers to a boron group substituted with alkyl having 1 to 40 carbon atoms, “arylboron group” refers to a boron group substituted with aryl having 6 to 60 carbon atoms, “arylphosphine group” refers to a phosphine group substituted with aryl having 6 to 60 carbon atoms, and “arylamine” refers to amine substituted with aryl having 6 to 60 carbon atoms.

As used herein, the term “fused (e.g., condensed) ring” refers to a fused aliphatic ring, a fused aromatic ring, a fused heteroaliphatic ring, a fused heteroaromatic ring, or a combination thereof.

Such a compound represented by Chemical Formula 1 of the present disclosure may be synthesized in various manners with reference to the synthesis process of embodiments described below. The detailed synthesis process will be described below in synthesis embodiments.

### <Organic Electroluminescent Device>

The present disclosure provides an organic electroluminescent device (“EL device”) including the compound represented by Chemical Formula 1.

More specifically, the organic EL device according to the present disclosure includes an anode, a cathode, and one or more organic layers disposed (e.g., interposed) between the anode and the cathode, and at least one of the one or more organic layers include the compound represented by Chemical Formula 1. In such a case, the compound may be used solely or as a combination of two or more kinds thereof.

The one or more organic layers may be any one or more of a hole injection layer, a hole transporting layer, a light emitting auxiliary layer, a light emitting layer, an electron transport auxiliary layer, an electron transporting layer, and an electron injection layer, and at least one of the organic layers may include the compound represented by Chemical Formula 1. Specifically, the organic layer including the compound represented by Chemical Formula 1 may preferably be a light emitting layer, an electron transport auxiliary layer, and/or an electron transporting layer.

The light emitting layer of the organic EL device of the present disclosure may include a host material (preferably, a phosphorescent host material). In addition, the light emitting layer of the organic EL device of the present disclosure may include, as a host, a compound other than the compound represented by Chemical Formula 1.

A structure of the organic EL device of the present disclosure is not particularly limited, but a non-limiting example thereof may have a structure in which a substrate, an anode, a hole injection layer, a hole transporting layer, a light emitting auxiliary layer, a light emitting layer, an electron transporting layer and a cathode are sequentially laminated. In such a case, at least one of the hole injection layer, the hole transporting layer, the light emitting auxiliary layer, the light emitting layer, and the electron transporting layer may include the compound represented by Chemical Formula 1, and preferably, the light emitting layer or the electron transporting layer may include the compound represented by Chemical Formula 1. In such a case, an electron injection layer may further be laminated on the electron transporting layer. In addition, the structure of the organic EL device of the present disclosure may have a structure in which an electron transport auxiliary layer is provided in addition to the electrodes and the organic layers described above. In such an embodiment, one or more of the hole injection layer, the hole transporting layer, the light emitting auxiliary layer, the light emitting layer, the electron transport auxiliary layer and the electron transporting layer may include the compound represented by Chemical Formula 1, and preferably, the light emitting layer, the electron transport auxiliary layer or the electron transporting layer may include the compound represented by Chemical Formula 1.

Meanwhile, the organic EL device of the present disclosure may be manufactured by forming organic layers and electrodes with conventional materials and through conventional methods known in the art, except that one or more of the aforementioned organic layers include the compound represented by Chemical Formula 1.

The organic layers may be formed by a vacuum deposition method or a solution coating method. Examples of the solution coating method may include, but are not limited to, spin coating, dip coating, doctor blading, inkjet printing, thermal transfer or the like.

The substrate used for manufacturing the organic EL device of the present disclosure is not particularly limited, but silicon wafers, quartz, glass plates, metal plates, plastic films, sheets or the like may be used.

In addition, a material of the anode may include, but not limited to, a metal such as vanadium, chromium, copper, zinc and gold or an alloy thereof, a metal oxide such as zinc oxide, indium oxide, indium tin oxide (ITO) and indium zinc oxide (IZO); a combination of oxide with metal such as ZnO:Al or SnO₂:Sb; a conductive polymer such as polythiophene, poly(3-methylthiophene), poly [3,4-(ethylene-1,2-dioxy) thiophene](PEDT), polypyrrole or polyaniline; carbon black or the like.

In addition, a material of the cathode may include, but not limited to, a metal such as magnesium, calcium, sodium, potassium, titanium, indium, yttrium, lithium, gadolinium, aluminum, silver, tin and lead or an alloy thereof, a multi-layered material such as LiF/Al and LiO₂/Al or the like.

In addition, materials of the hole injection layer, the hole transporting layer and the light emitting auxiliary layer are not particularly limited and conventional materials known in the art may be used.

Hereinafter, the present disclosure will be described in detail through exemplary embodiments. However, the following embodiments are merely to illustrate the invention, and the present disclosure is not limited by the following embodiments.

### [Preparation Example 1] Synthesis of PPY-1

### <Step 1> Synthesis of PPY-1

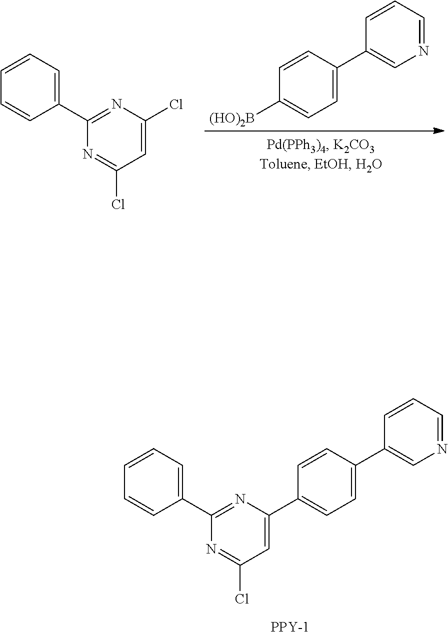

45.0 g of 4,6-dichloro-2-phenylpyrimidine and 40.0 g of (4-(pyridin-3-yl)phenyl)boronic acid, 6.0 g of tetrakis(phenylphosphine)palladium (0), and 42 g of K₂CO₃ were added to 800 ml of toluene, 200 ml of ethanol, and 200 ml of water, and the mixture was stirred and heated under reflux for 2 hours. After the reaction was completed, the solution was inactivated with a sufficient amount of water, and transferred to a separatory funnel, followed by extraction with methylene chloride. An organic layer was dried over magnesium sulfate, concentrated and purified by column chromatography, thereby obtaining 39.8 g (yield 58%) of PPY-1.

1H-NMR: δ 9.24 (s, 1H), 8.70 (d, 1H), 8.42-8.30 (m, 5H), 7.57-7.50 (m, 4H), 7.25 (d, 2H) 7.03 (s, 1H)

Mass: [(M+H)+]: 344

### [Preparation Example 2] Synthesis of PPY-2 and 3

### <Step 1> Synthesis of (E)-1-(4-bromophenyl)-3-(4-pyridin-3-yl)phenyl)prop-2-ene-1-one

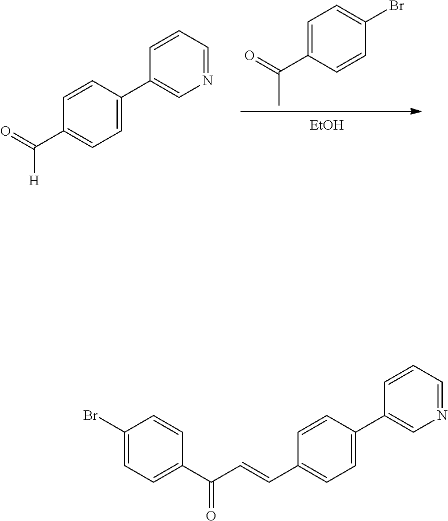

50.0 g of 4-(pyridin-3-yl) benzaldehyde, 49.1 g of 1-(4-bromophenyl)ethan-1-one, and 18.2 g of sodium methoxide were added to 800 ml of ethanol, and the mixture was stirred for 8 hours. After the reaction was completed, the mixture was stirred at room temperature for 1 hour, followed by extraction with ethyl acetate. An organic layer was dried over magnesium sulfate, concentrated and purified by column chromatography, thereby obtaining 36.4 g (yield 72%) of (E)-1-(4-bromophenyl)-3-(4-pyridin-3-yl)phenyl)prop-2-ene-1-one.

1H-NMR: δ 9.24 (s, 1H), 8.50 (d, 1H), 8.38 (d, 1H), 8.08-8.01 (m, 3H), 7.75 (d, 2H), 7.60-7.45 (m, 6H)

Mass: [(M+H)+]: 364

### <Step 2> Synthesis of PPY-2

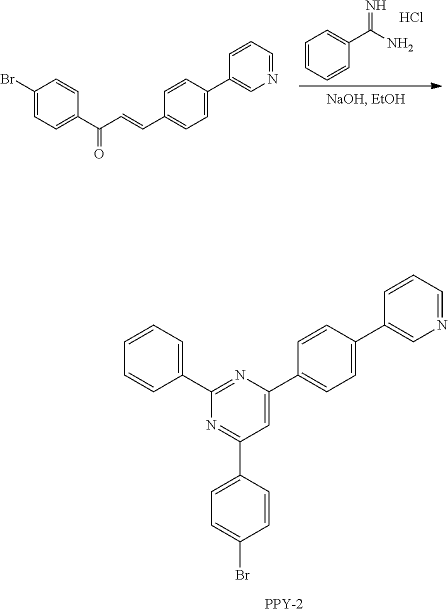

36.4 g of (E)-1-(4-bromophenyl)-3-(4-pyridin-3-yl)phenyl)prop-2-ene-1-one, 24.1 g of benzimidamide hydrochloride, 14.2 g of sodium hydroxide were added to 500 ml of ethanol, and the mixture was stirred and heated under reflux for 4 hours. After the reaction was completed, the reaction product was concentrated under reduced pressure to 250 ml, inactivated with a sufficient amount of water, and then transferred to a separatory funnel, followed by extraction with methylene chloride. An organic layer was dried over magnesium sulfate, concentrated, and purified by column chromatography, thereby obtaining 36.2 g (yield 79%) of PPY-2.

1H-NMR: δ 9.21 (s, 1H), 8.70 (d, 1H), 8.42-8.30 (m, 6H), 7.76 (d, 2H), 7.59-7.55 (m, 6H), 7.25 (d, 2H)

Mass: [(M+H)+]: 464

### <Step 3> Synthesis of PPY-3

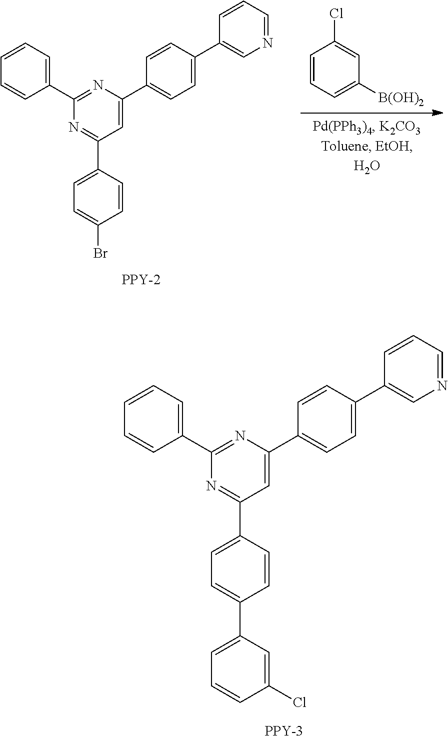

15.0 g of PPY-2, 6.1 g of (3-chlorophenyl)boronic acid, 0.9 g of tetrakis(phenylphosphine)palladium (0), and 7.0 g of K₂CO₃ were added to 300 ml of toluene, 60 ml of ethanol, and 60 ml of water, and the mixture was stirred and heated under reflux for 2 hours. After the reaction was completed, the solution was inactivated with a sufficient amount of water, and transferred to a separatory funnel, followed by extraction with methylene chloride. An organic layer was dried over magnesium sulfate, concentrated and purified by column chromatography, thereby obtaining 10.9 g (yield 68%) of PPY-3.

1H-NMR: δ 9.21 (s, 1H), 8.70 (d, 1H), 8.42-8.30 (m, 6H), 7.97 (s, 1H), 7.76 (d, 2H), 7.59-7.55 (m, 6H), 7.48 (m, 2H), 7.39 (d, 1H), 7.25 (d, 2H)

Mass: [(M+H)+]: 496

### [Preparation Example 3] Synthesis of PPY-4 to 6

### <Step 1> Synthesis of (E)-1-(3-bromophenyl)-3-(4-pyridin-3-yl)phenyl)prop-2-ene-1-one

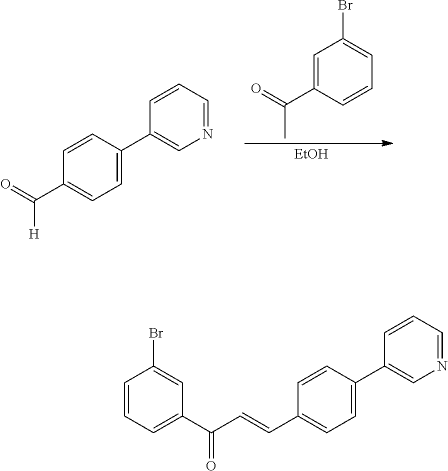

50.0 g of 4-(pyridin-3-yl) benzaldehyde, 49.1 g of 1-(3-bromophenyl)ethan-1-one, and 18.2 g of sodium methoxide were added to 800 ml of ethanol, and the mixture was stirred for 8 hours. After the reaction was completed, the mixture was stirred at room temperature for 1 hour, followed by extraction with ethyl acetate. An organic layer was dried over magnesium sulfate, concentrated and purified by column chromatography, thereby obtaining 38.2 g (yield 74%) of (E)-1-(3-bromophenyl)-3-(4-pyridin-3-yl)phenyl)prop-2-ene-1-one.

1H-NMR: δ 9.24 (s, 1H), 8.50 (d, 1H), 8.38 (d, 1H), 8.08-8.01 (m, 3H), 7.82 (d, 1H), 7.60-7.45 (m, 7H)

Mass: [(M+H)+]: 364

### <Step 2> Synthesis of PPY-4

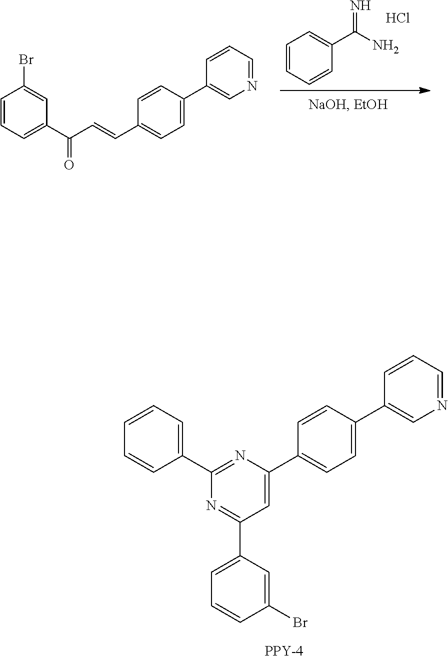

38.2 g of (E)-1-(3-bromophenyl)-3-(4-pyridin-3-yl)phenyl)prop-2-ene-1-one, 25.0 g of benzimidamide hydrochloride, and 14.8 g of sodium hydroxide were added to 500 ml of ethanol, and the mixture was stirred and heated under reflux for 4 hours. After the reaction was completed, the reaction product was concentrated under reduced pressure to 250 ml, inactivated with a sufficient amount of water, and then transferred to a separatory funnel, followed by extraction with methylene chloride. An organic layer was dried over magnesium sulfate, concentrated, and purified by column chromatography, thereby obtaining 34.2 g (yield 75%) of PPY-4.

1H-NMR: δ 9.24 (s, 1H), 8.70 (d, 1H), 8.42-8.30 (m, 6H), 7.78 (d, 1H), 7.67 (d, 1H) 7.50-7.43 (m, 6H), 7.25 (d, 2H)

Mass: [(M+H)+]: 464

### <Step 3> Synthesis of PPY-5

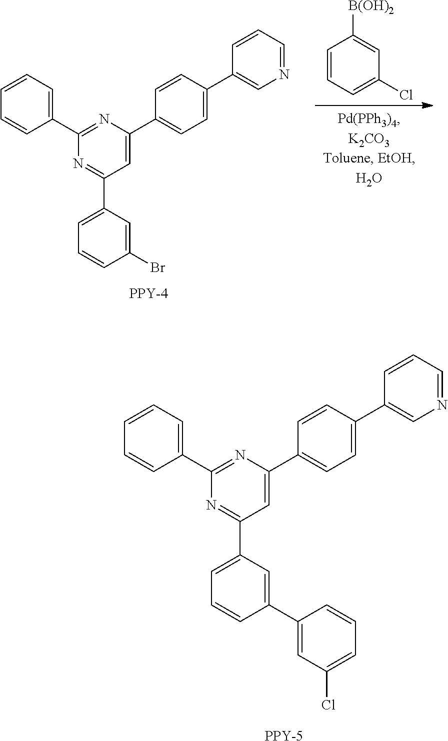

15.0 g of PPY-4, 6.1 g of (3-chlorophenyl)boronic acid, 0.9 g of tetrakis(phenylphosphine)palladium (0), and 7.0 g of of K₂CO₃ were added to 300 ml of toluene, 60 ml of ethanol, and 60 ml of water, and the mixture was stirred and heated under reflux for 2 hours. After the reaction was completed, the solution was inactivated with a sufficient amount of water, and transferred to a separatory funnel, followed by extraction with methylene chloride. An organic layer was dried over magnesium sulfate, concentrated and purified by column chromatography, thereby obtaining 10.1 g (yield 67%) of PPY-5.

1H-NMR: δ 9.24 (s, 1H), 8.70 (d, 1H), 8.42-8.30 (m, 6H), 7.97 (s, 1H), 7.78 (d, 1H), 7.67 (d, 1H) 7.50-7.43 (m, 8H), 7.35 (d, 1H), 7.25 (d, 2H)

Mass: [(M+H)+]: 496

### <Step 4> Synthesis of PPY-6

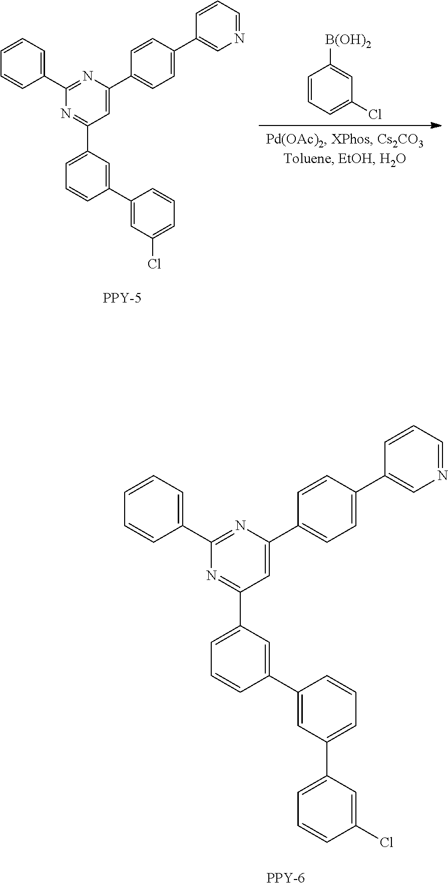 [SMILES: Clc1cccc(-c2cccc(-c3cc(-c4ccc(-c5cccnc5)cc4)nc(-c4ccccc4)n3)c2)c1 ; OB(O)c1cccc(Cl)c1 ; "PPY-5" ; Clc1cccc(-c2cccc(-c3cccc(-c4cc(-c5ccc(-c6cccnc6)cc5)nc(-c5ccccc5)n4)c3)c2)c1 ; "PPY-6"]

10.0 g of PPY-5, 4.1 g of (3-chlorophenyl)boronic acid, 0.1 g of Pd(OAc)₂, 0.4 g of Xphos, and 4.5 g of Cs₂CO₃ were added to 200 ml of toluene, 40 ml of ethanol, and 40 ml of water, and the mixture was stirred and heated under reflux for 2 hours. After the reaction was completed, the solution was inactivated with a sufficient amount of water, and transferred to a separatory funnel, followed by extraction with methylene chloride. An organic layer was dried over magnesium sulfate, concentrated and purified by column chromatography, thereby obtaining 6.7 g (yield 66%) of PPY-6.

1H-NMR: δ 9.24 (s, 1H), 8.70 (d, 1H), 8.42-8.30 (m, 6H), 7.97 (s, 1H), 7.90 (s, 1H), 7.78 (d, 1H), 7.67 (d, 1H) 7.50-7.40 (m, 10H), 7.35 (d, 2H), 7.25 (d, 2H)

Mass: [(M+H)+]: 572

### [Preparation Example 4] Synthesis of PPY-7 and 8

### <Step 1> Synthesis of PPY-7

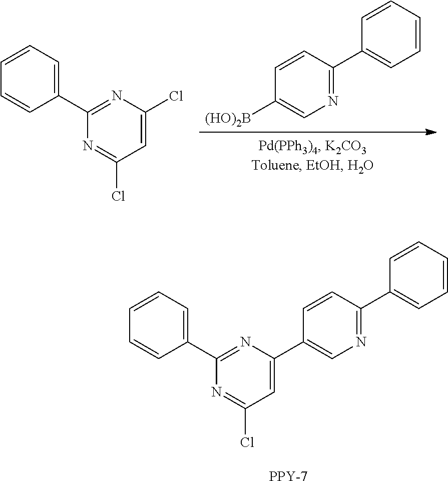

45.0 g of 4,6-dichloro-2-phenylpyrimidine, 38.7 g of (6-phenylpyridin-3-yl)boronic acid, 6.0 g of tetrakis(phenylphosphine)palladium (0), and 42 g of K₂CO₃ were added to 800 ml of toluene, 200 ml of ethanol, and 200 ml of water, and the mixture was stirred and heated under reflux for 2 hours. After the reaction was completed, the solution was inactivated with a sufficient amount of water, and transferred to a separatory funnel, followed by extraction with methylene chloride. An organic layer was dried over magnesium sulfate, concentrated and purified by column chromatography, thereby obtaining 40.7 g (yield 61%) of PPY-7.

1H-NMR: δ 9.23 (s, 1H), 8.62 (d, 1H), 8.42-8.30 (m, 3H), 7.96 (d, 2H), 7.73 (s, 1H), 7.54-7.48 (m, 4H), 7.31 (d, 2H)

Mass: [(M+H)+]: 344

### <Step 2> Synthesis of PPY-8

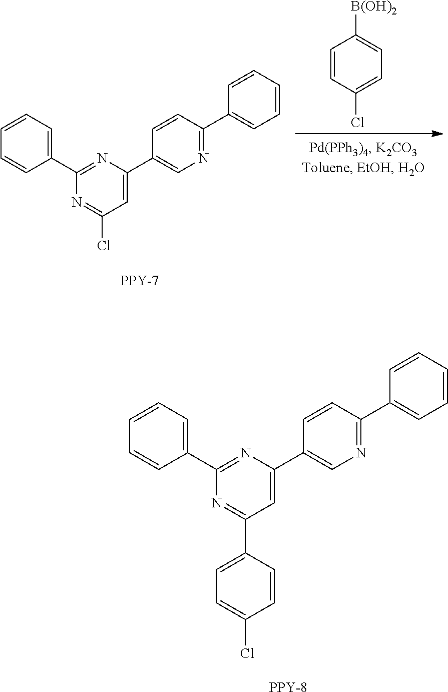

15.0 g of PPY-7, 6.1 g of (3-chlorophenyl)boronic acid, 0.9 g of tetrakis(phenylphosphine)palladium (0), and 7.1 g of K₂CO₃ were added to 300 ml of toluene, 60 ml of ethanol, and 60 ml of water, and the mixture was stirred and heated under reflux for 2 hours. After the reaction was completed, the solution was inactivated with a sufficient amount of water, and transferred to a separatory funnel, followed by extraction with methylene chloride. An organic layer was dried over magnesium sulfate, concentrated and purified by column chromatography, thereby obtaining 13.7 g (yield 72%) of PPY-8.

1H-NMR: δ 9.15 (s, 1H), 8.73 (d, 1H), 8.43-8.12 (m, 4H), 8.13 (s, 1H), 7.99-7.97 (m, 3H), 7.52-7.41 (m, 6H), 7.11 (d, 2H)

Mass: [(M+H)+]: 420

### [Preparation Example 5] Synthesis of PPY-1 and 2

### <Step 1> Synthesis of PTZ-1

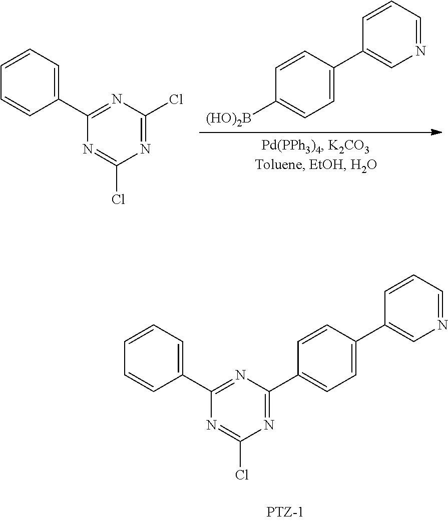 [SMILES: Clc1nc(Cl)nc(-c2ccccc2)n1 ; OB(O)c1ccc(-c2cccnc2)cc1 ; Clc1nc(-c2ccccc2)nc(-c2ccc(-c3cccnc3)cc2)n1]

45.0 g of 2,4-dichloro-6-phenyl-1,3,5-triazine, 39.2 g of (4-(pyridin-3-yl)phenyl)boronic acid, 6.0 g of tetrakis(phenylphosphine)palladium (0), and 42 g of K₂CO₃ were added to 800 ml of toluene, 200 ml of ethanol, and 200 ml of water, and the mixture was stirred and heated under reflux for 2 hours. After the reaction was completed, the solution was inactivated with a sufficient amount of water, and transferred to a separatory funnel, followed by extraction with methylene chloride. An organic layer was dried over magnesium sulfate, concentrated and purified by column chromatography, thereby obtaining 36.2 g (yield 53%) of PTZ-1.

1H-NMR: δ 9.24 (s, 1H), 8.70 (d, 1H), 8.42-8.30 (m, 3H), 7.96 (d, 2H), 7.57-7.50 (m, 4H), 7.25 (d, 2H)

Mass: [(M+H)+]: 345

### <Step 2> Synthesis of PTZ-2

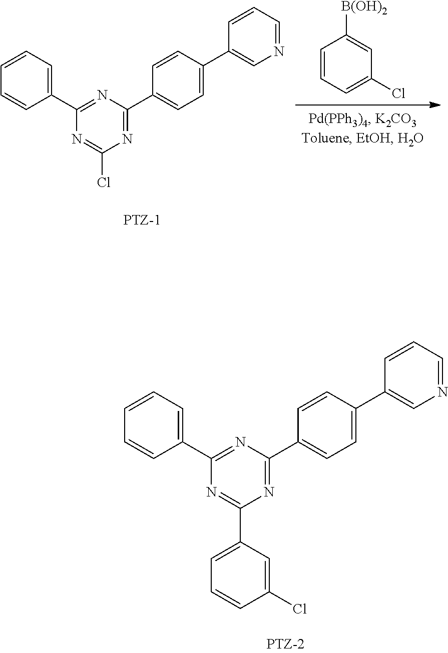 [SMILES: Clc1nc(-c2ccccc2)nc(-c2ccc(-c3cccnc3)cc2)n1 ; OB(O)c1cccc(Cl)c1 ; Clc1cccc(-c2nc(-c3ccccc3)nc(-c3ccc(-c4cccnc4)cc3)n2)c1]

10.0 g of PTZ-1, 4.1 g of (3-chlorophenyl)boronic acid, 0.6 g of tetrakis(phenylphosphine)palladium (0), and 4.7 g of K₂CO₃ were added to 200 ml of toluene, 40 ml of ethanol, and 40 ml of water, and the mixture was stirred and heated under reflux for 2 hours. After the reaction was completed, the solution was inactivated with a sufficient amount of water, and transferred to a separatory funnel, followed by extraction with methylene chloride. An organic layer was dried over magnesium sulfate, concentrated and purified by column chromatography, thereby obtaining 8.7 g (yield 71%) of PTZ-2.

1H-NMR: δ 9.24 (s, 1H), 8.70 (d, 1H), 8.42-8.30 (m, 3H), 8.16 (s, 1H), 7.96-7.95 (m, 3H), 7.50-7.43 (m, 6H), 7.25 (d, 2H)

Mass: [(M+H)+]: 421

### [Preparation Example 6] Synthesis of PTZ-3

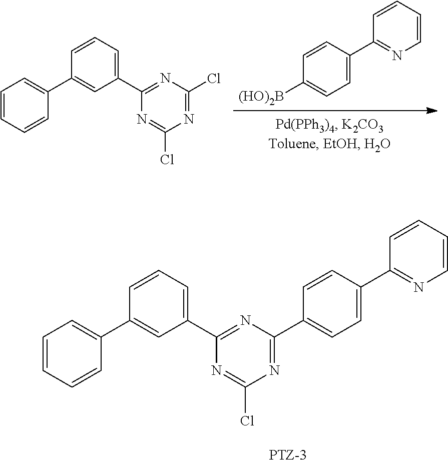 [SMILES: Clc1nc(Cl)nc(-c2cccc(-c3ccccc3)c2)n1 ; OB(O)c1ccc(-c2ccccn2)cc1 ; Clc1nc(-c2ccc(-c3ccccn3)cc2)nc(-c2cccc(-c3ccccc3)c2)n1 ; "PTZ-3"]

45.0 g of 2-([1,1′-biphenyl]-3-yl)-4,6-dichloro-1,3,5-triazine, 38.1 g of (4-(pyridin-2-yl)phenyl)boronic acid, 6.0 g of tetrakis(phenylphosphine)palladium (0), and 42 g of K₂CO₃ were added to 800 ml of toluene, 200 ml of ethanol, and 200 ml of water, and the mixture was stirred and heated under reflux for 2 hours. After the reaction was completed, the solution was inactivated with a sufficient amount of water, and transferred to a separatory funnel, followed by extraction with methylene chloride. An organic layer was dried over magnesium sulfate, concentrated and purified by column chromatography, thereby obtaining 40.4 g (yield 65%) of PTZ-3.

1H-NMR: δ 9.23 (s, 1H), 8.70 (d, 1H), 8.42-8.30 (m, 3H), 7.96 (d, 2H), 7.75 (d, 2H) 7.67-7.43 (m, 7H), 7.23 (d, 2H)

Mass: [(M+H)+]: 421

### [Synthesis Example 1] Synthesis of Compound 1

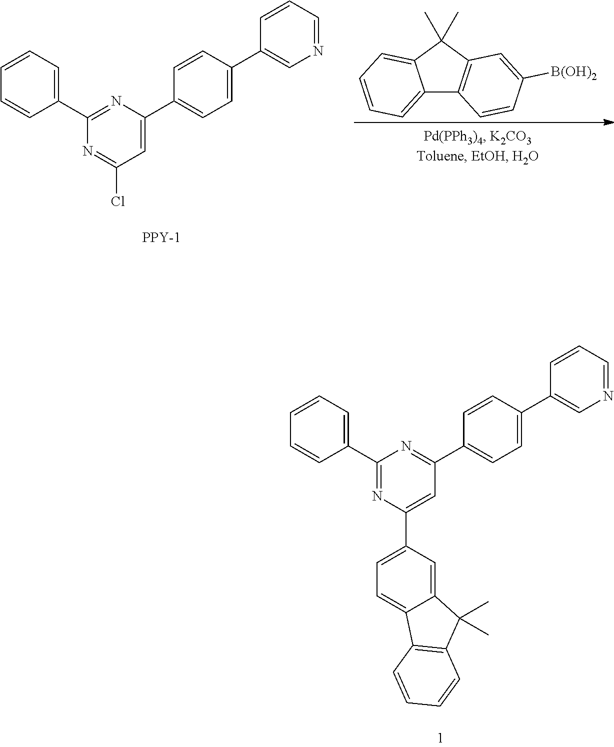

3.0 g of PPY-1, 4.3 g of (9,9-dimethyl-9H-fluoren-2-yl)boronic acid, and 3.3 g of K₂CO₃ were mixed, 60 ml of toluene, 12 ml of ethanol, and 12 ml of water were added thereto, 500 mg of tetrakis(phenylphosphine)palladium (0) was further added thereto, and the mixture was stirred and heated for 4 hours. After the reaction was completed, the temperature was lowered to room temperature, and the reaction product was filtered. The filtrate was poured into water, followed by extraction with chloroform. An organic layer was dried over MgSO₄ and concentrated under reduced pressure, and column chromatography with MC:Hex=2:1 was performed, thereby obtaining 2.8 g (yield 55%) of Compound 1 in a white solid state.

Mass: [(M+H)+]: 502

### [Synthesis Example 2] Synthesis of Compound 2

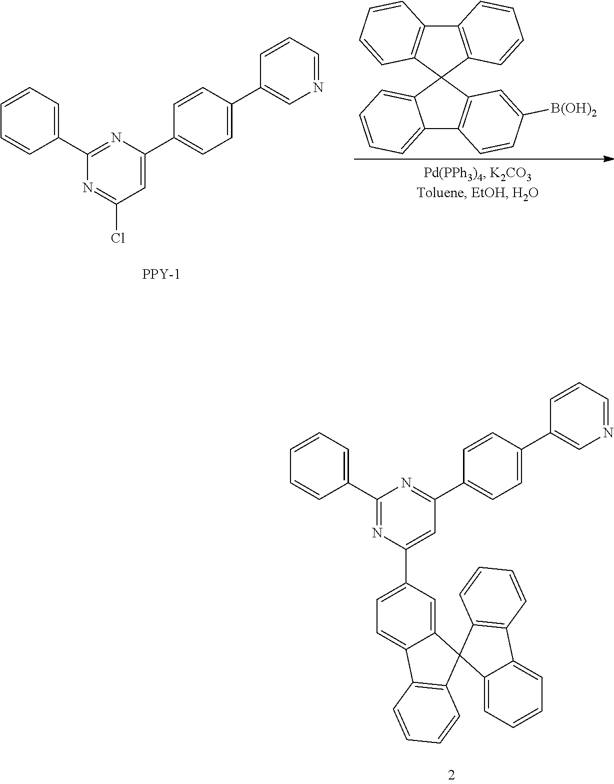

3.0 g of PPY-1, 5.1 g of 9,9′-spirobi[fluorene]-2-yl boronic acid, and 3.3 g of K₂CO₃ were mixed, 60 ml of toluene, 12 ml of ethanol, and 12 ml of water were added thereto, 500 mg of tetrakis(phenylphosphine)palladium (0) was further added thereto, and the mixture was stirred and heated for 4 hours. After the reaction was completed, the temperature was lowered to room temperature, and the reaction product was filtered. The filtrate was poured into water, followed by extraction with chloroform. An organic layer was dried over MgSO₄ and concentrated under reduced pressure, and column chromatography with MC:Hex=2:1 was performed, thereby obtaining 3.2 g (yield 58%) of Compound 2 in a white solid state.

Mass: [(M+H)+]: 624

### [Synthesis Example 3] Synthesis of Compound 4

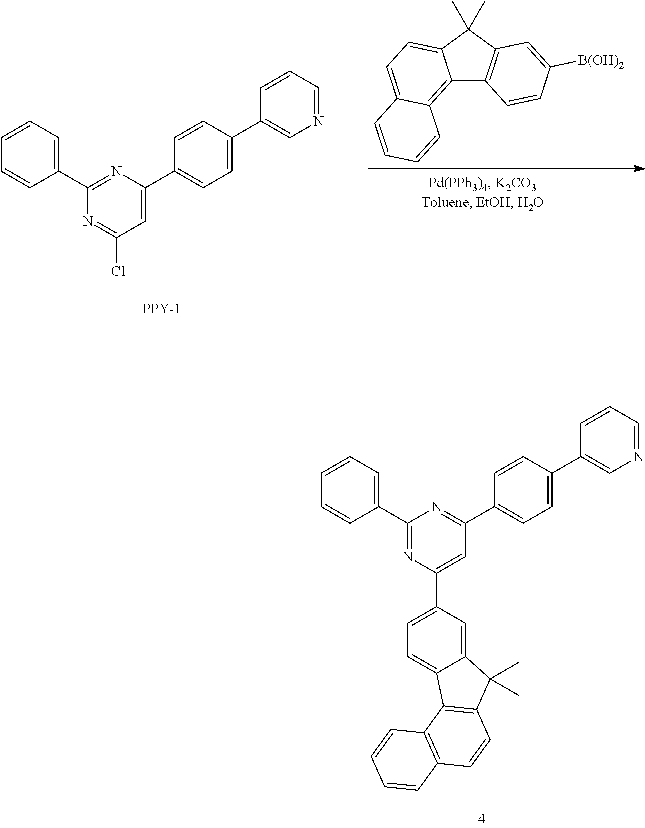

3.1 g of PPY-1, 4.8 g of (7,7-dimethyl-7H-benzo[c]fluoren-9-yl)boronic acid, and 3.3 g of K₂CO₃ were mixed, 60 ml of toluene, 12 ml of ethanol, and 12 ml of water were added thereto, 500 mg of tetrakis(phenylphosphine)palladium (0) was further added thereto, and the mixture was stirred and heated for 4 hours. After the reaction was completed, the temperature was lowered to room temperature, and the reaction product was filtered. The filtrate was poured into water, followed by extraction with chloroform. An organic layer was dried over MgSO₄ and concentrated under reduced pressure, and column chromatography with MC:Hex=2:1 was performed, thereby obtaining 3.5 g (yield 56%) of Compound 4 in a white solid state.

Mass: [(M+H)+]: 551

### [Synthesis Example 4] Synthesis of Compound 42

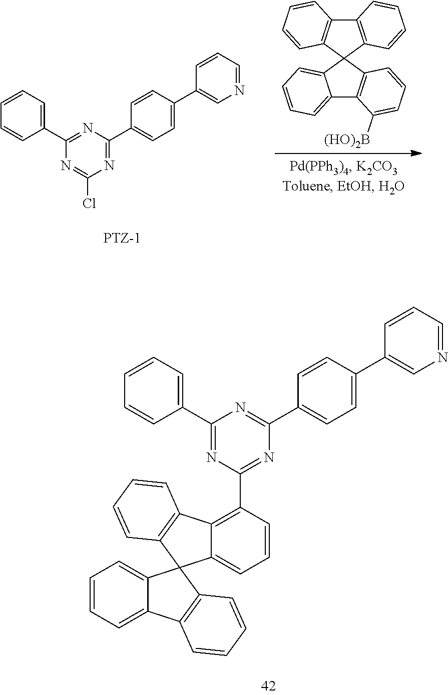 [SMILES: Clc1nc(-c2ccccc2)nc(-c2ccc(-c3cccnc3)cc2)n1 ; OB(O)c1cccc2c1-c1ccccc1C21c2ccccc2-c2ccccc21 ; "PTZ-1" ; c1ccc(-c2nc(-c3ccc(-c4cccnc4)cc3)nc(-c3cccc4c3-c3ccccc3C43c4ccccc4-c4ccccc43)n2)cc1]

3.0 g of PTZ-1, 5.1 g of 9,9′-spirobi[fluorene]-4-yl boronic acid, and 3.3 g of K₂CO₃ were mixed, 60 ml of toluene, 12 ml of ethanol, and 12 ml of water were added thereto, 500 mg of tetrakis(phenylphosphine)palladium (0) was further added thereto, and the mixture was stirred and heated for 4 hours. After the reaction was completed, the temperature was lowered to room temperature, and the reaction product was filtered. The filtrate was poured into water, the resultant solid was filtered out, the resultant solid was dissolved in a sufficient amount of MC and concentrated under reduced pressure, and column chromatography with MC:Hex=2:1 was performed, thereby obtaining 4.1 g (yield 75%) of Compound 42 in a white solid state.

Mass: [(M+H)+]: 625

### [Synthesis Example 5] Synthesis of Compound 45

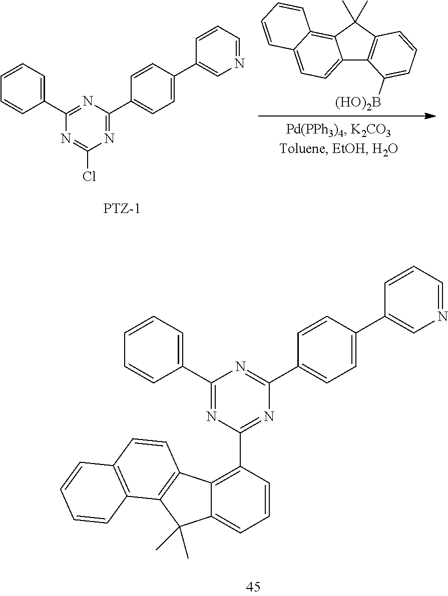

3.2 g of PTZ-1, 4.9 g of (7,7-dimethyl-7H-benzo[c]fluoren-7-yl)boronic acid, and 3.3 g of 5 K₂CO₃ were mixed, 60 ml of toluene, 12 ml of ethanol, and 12 ml of water were added thereto, 520 mg of tetrakis(phenylphosphine)palladium (0) was further added thereto, and the mixture was stirred and heated for 4 hours. After the reaction was completed, the temperature was lowered to room temperature, and the reaction product was filtered. The filtrate was poured into water, followed by extraction with chloroform. An organic layer was dried over MgSO₄ and concentrated under reduced pressure, and column chromatography with MC:Hex=2:1 was performed, thereby obtaining 3.8 g (yield 57%) of Compound 45 in a white solid state.

Mass: [(M+H)+]: 553

### [Synthesis Example 6] Synthesis of Compound 111

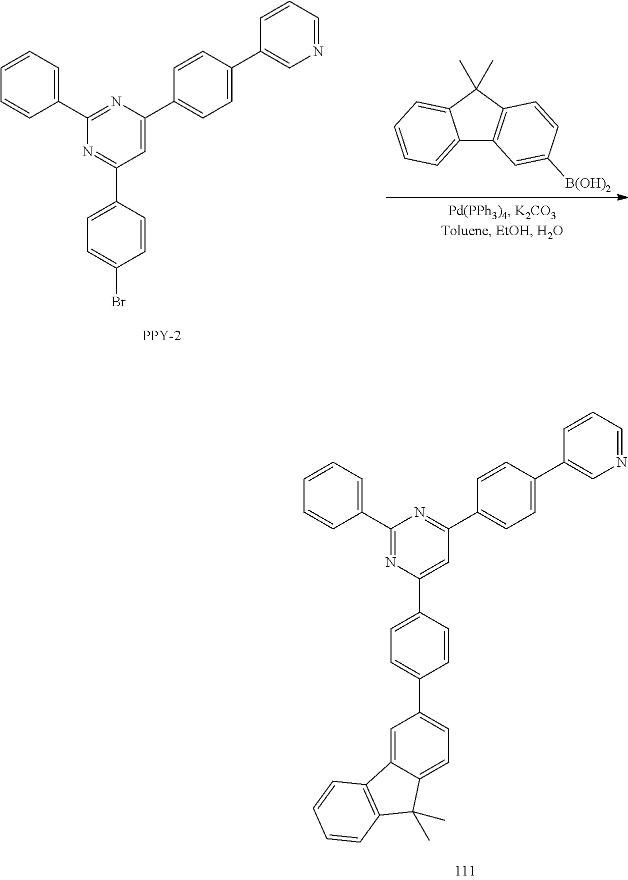

2.0 g of PPY-2, 2.1 g of (9,9-dimethyl-9H-fluoren-3-yl)boronic acid, and 1.8 g of K₂CO₃ were mixed, 50 ml of toluene, 10 ml of ethanol, and 10 ml of water were added thereto, 200 mg of tetrakis(phenylphosphine)palladium (0) was further added thereto, and the mixture was stirred and heated for 4 hours. After the reaction was completed, the temperature was lowered to room temperature, and the reaction product was filtered. The filtrate was poured into water, followed by extraction with chloroform. An organic layer was dried over MgSO₄ and concentrated under reduced pressure, and column chromatography with MC was performed, thereby obtaining 1.8 g (yield 76%) of Compound 111 in a white solid state.

Mass: [(M+H)+]: 578

### [Synthesis Example 7] Synthesis of Compound 112

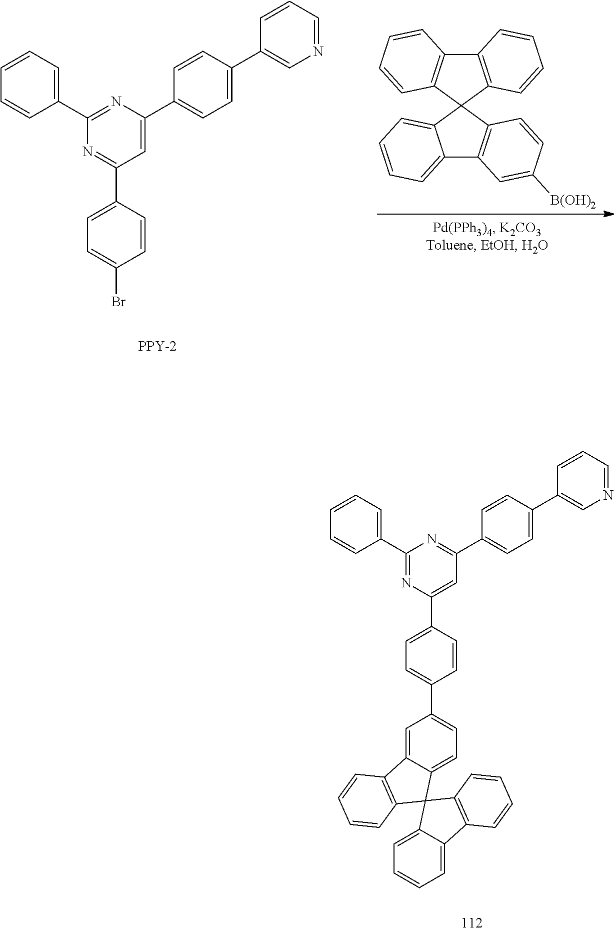 [SMILES: Brc1ccc(-c2cc(-c3ccc(-c4cccnc4)cc3)nc(-c3ccccc3)n2)cc1 ; OB(O)c1ccc2c(c1)-c1ccccc1C21c2ccccc2-c2ccccc21 ; "PPY-2" ; c1ccc(-c2nc(-c3ccc(-c4cccnc4)cc3)cc(-c3ccc(-c4ccc5c(c4)-c4ccccc4C54c5ccccc5-c5ccccc54)cc3)n2)cc1]

2.0 g of PPY-2, 2.5 g of 9,9′-spirobi[fluorene]-3-yl boronic acid, and 2.0 g of K₂CO₃ were mixed, 50 ml of toluene, 12 ml of ethanol, and 12 ml of water were added thereto, 200 mg of tetrakis(phenylphosphine)palladium (0) was further added thereto, and the mixture was stirred and heated for 4 hours. After the reaction was completed, the temperature was lowered to room temperature, and the reaction product was filtered. The filtrate was poured into water, followed by extraction with chloroform. An organic layer was dried over MgSO₄ and concentrated under reduced pressure, and column chromatography with THF:Hex=1:5 was performed, thereby obtaining 1.5 g (yield 55%) of Compound 112 in a white solid state.

Mass: [(M+H)+]: 700

### [Synthesis Example 8] Synthesis of Compound 121

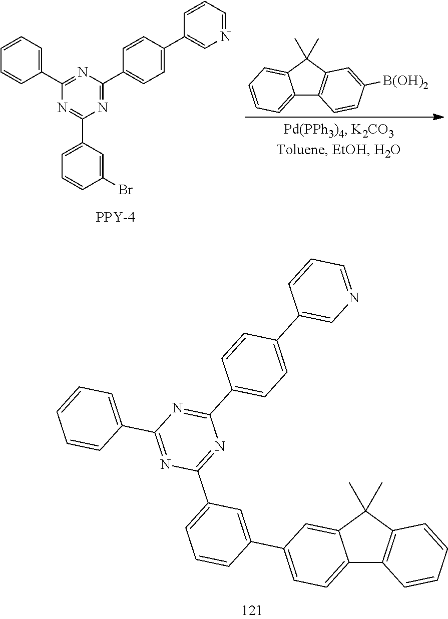

2.1 g of PPY-4, 2.2 g of (9,9-dimethyl-9H-fluoren-2-yl)boronic acid, and 1.9 g of K₂CO₃ were mixed, 50 ml of toluene, 10 ml of ethanol, and 10 ml of water were added thereto, 220 mg of tetrakis(phenylphosphine)palladium (0) was further added thereto, and the mixture was stirred and heated for 4 hours. After the reaction was completed, the temperature was lowered to room temperature, and the reaction product was filtered. The filtrate was poured into water, followed by extraction with chloroform. An organic layer was dried over MgSO₄ and concentrated under reduced pressure, and column chromatography with MC was performed, thereby obtaining 1.6 g (yield 72%) of Compound 121 in a white solid state.

Mass: [(M+H)+]: 578

### [Synthesis Example 9] Synthesis of Compound 133

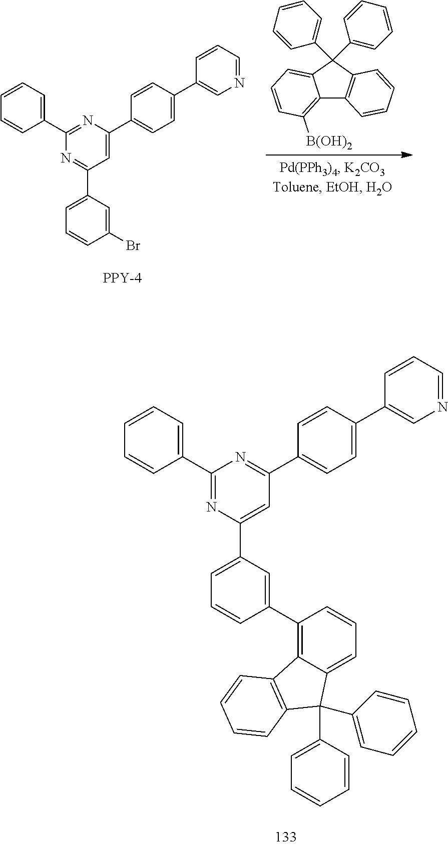

2.1 g of PPY-4, 2.7 g of (9,9-diphenyl-9H-fluoren-4-yl)boronic acid, and 2.1 g of K₂CO₃ were mixed, 50 ml of toluene, 12 ml of ethanol, and 12 ml of water were added thereto, 210 mg of tetrakis(phenylphosphine)palladium (0) was further added thereto, and the mixture was stirred and heated for 4 hours. After the reaction was completed, the temperature was lowered to room temperature, and the reaction product was filtered. The filtrate was poured into water, followed by extraction with chloroform. An organic layer was dried over MgSO₄ and concentrated under reduced pressure, and column chromatography with MC added with some pyridine was performed, thereby obtaining 2.1 g (yield 68%) of Compound 133 in a white solid state.

Mass: [(M+H)+]: 702

### [Synthesis Example 10] Synthesis of Compound 151

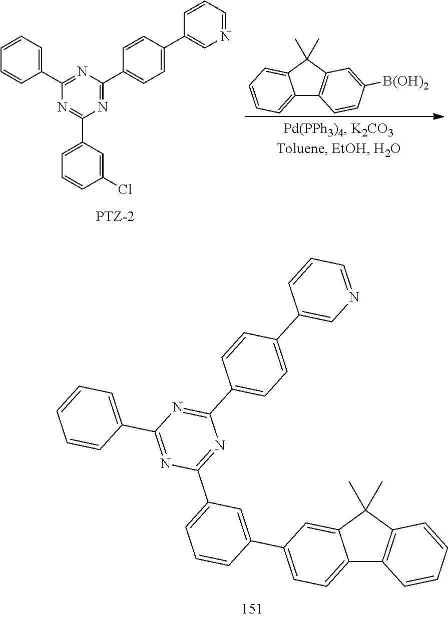 [SMILES: Clc1cccc(-c2nc(-c3ccccc3)nc(-c3ccc(-c4cccnc4)cc3)n2)c1 ; CC1(C)c2ccccc2-c2ccc(B(O)O)cc21 ; CC1(C)c2ccccc2-c2ccc(-c3cccc(-c4nc(-c5ccccc5)nc(-c5ccc(-c6cccnc6)cc5)n4)c3)cc21]

2.3 g of PTZ-2, 2.3 g of (9,9-dimethyl-9H-fluoren-2-yl)boronic acid, and 3.0 g of Cs₂CO₃ were mixed, 60 ml of toluene, 12 ml of ethanol, and 12 ml of water were added thereto, 50 mg of Pd(OAc)₂ and 230 mg of Xphos were further added thereto, and the mixture was stirred and heated for 4 hours. After the reaction was completed, the temperature was lowered to room temperature, and the reaction product was filtered. The filtrate was poured into water, followed by extraction with chloroform. An organic layer was dried over MgSO₄ and concentrated under reduced pressure, and column chromatography with MC was performed, thereby obtaining 2.2 g (yield 75%) of Compound 151 in a white solid state.

Mass: [(M+H)+]: 579

### [Synthesis Example 11] Synthesis of Compound 156

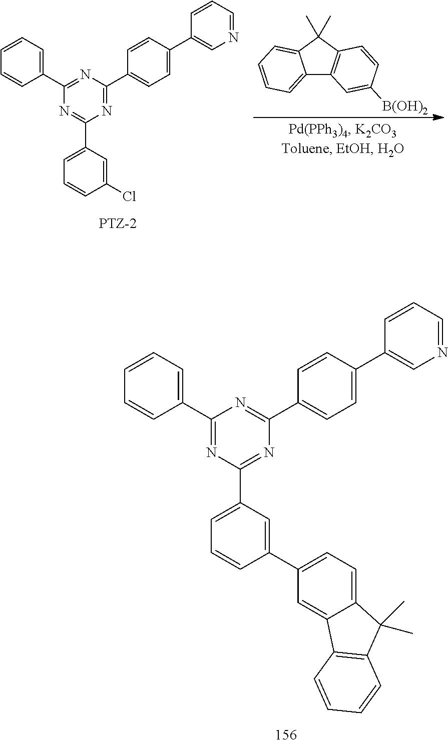 [SMILES: Clc1cccc(-c2nc(-c3ccccc3)nc(-c3ccc(-c4cccnc4)cc3)n2)c1 ; CC1(C)c2ccccc2-c2cc(B(O)O)ccc21 ; CC1(C)c2ccccc2-c2cc(-c3cccc(-c4nc(-c5ccccc5)nc(-c5ccc(-c6cccnc6)cc5)n4)c3)ccc21]

2.1 g of PTZ-2, 2.2 g of (9,9-dimethyl-9H-fluoren-3-yl)boronic acid, and 2.8 g of Cs₂CO₃ were mixed, 60 ml of toluene, 12 ml of ethanol, and 12 ml of water were added thereto, 48 mg of Pd(OAc)₂ and 200 mg of Xphos were further added thereto, and the mixture was stirred and heated for 4 hours. After the reaction was completed, the temperature was lowered to room temperature, and the reaction product was filtered. The filtrate was poured into water, followed by extraction with chloroform. An organic layer was dried over MgSO₄ and concentrated under reduced pressure, and column chromatography with MC was performed, thereby obtaining 2.0 g (yield 71%) of Compound 156 in a white solid state.

Mass: [(M+H)+]: 579

### [Synthesis Example 12] Synthesis of Compound 346

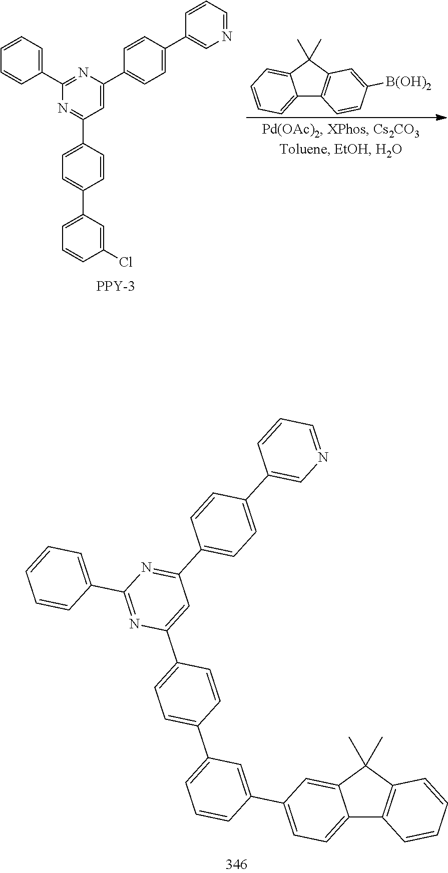

2.5 g of PPY-3, 2.4 g of (9,9-dimethyl-9H-fluoren-2-yl)boronic acid, and 3.3 g of Cs₂CO₃ were mixed, 60 ml of toluene, 12 ml of ethanol, and 12 ml of water were added thereto, 57 mg of Pd(OAc)₂ and 250 mg of Xphos were further added thereto, and the mixture was stirred and heated for 4 hours. After the reaction was completed, the temperature was lowered to room temperature, and the reaction product was filtered. The filtrate was poured into water, followed by extraction with chloroform. An organic layer was dried over MgSO₄ and concentrated under reduced pressure, and column chromatography with MC was performed, thereby obtaining 2.3 g (yield 70%) of Compound 346 in a white solid state.

Mass: [(M+H)+]: 654

### [Synthesis Example 13] Synthesis of Compound 350

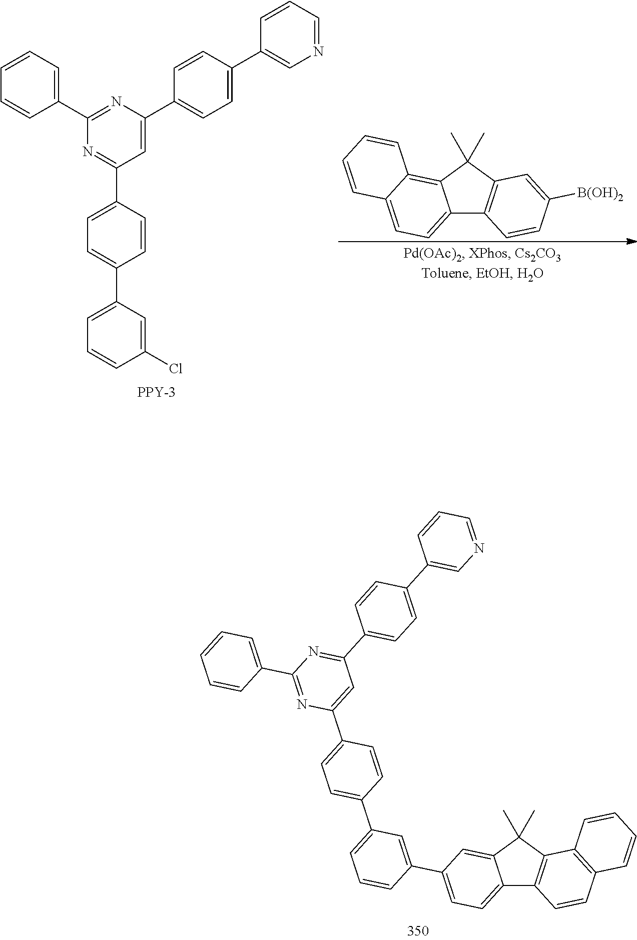

2.5 g of PPY-3, 2.8 g of (7,7-dimethyl-7H-benzo[c]fluoren-9-yl)boronic acid, and 3.3 g of Cs₂CO₃ were mixed, 60 ml of toluene, 12 ml of ethanol, and 12 ml of water were added thereto, 57 mg of Pd(OAc)₂ and 250 mg of Xphos were further added thereto, and the mixture was stirred and heated for 4 hours. After the reaction was completed, the temperature was lowered to room temperature, and the reaction product was filtered. The filtrate was poured into water, followed by extraction with chloroform. An organic layer was dried over MgSO₄ and concentrated under reduced pressure, and column chromatography with MC was performed, thereby obtaining 2.5 g (yield 71%) of Compound 350 in a white solid state.

Mass: [(M+H)+]: 704

### [Synthesis Example 14] Synthesis of Compound 376

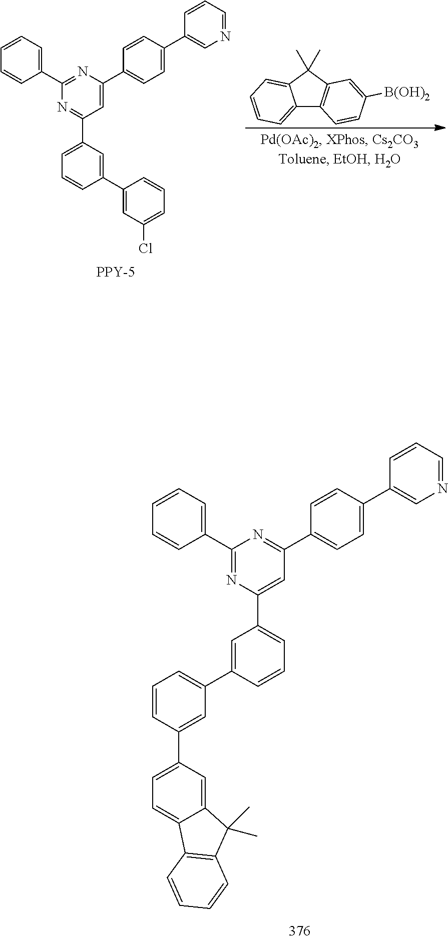 [SMILES: Clc1cccc(-c2cccc(-c3cc(-c4ccc(-c5cccnc5)cc4)nc(-c4ccccc4)n3)c2)c1 ; CC1(C)c2ccccc2-c2ccc(B(O)O)cc21 ; "PPY-5" ; CC1(C)c2ccccc2-c2ccc(-c3cccc(-c4cccc(-c5cc(-c6ccc(-c7cccnc7)cc6)nc(-c6ccccc6)n5)c4)c3)cc21]

2.2 g of PPY-5, 2.3 g of (9,9-dimethyl-9H-fluoren-2-yl)boronic acid, and 3.0 g of Cs₂CO₃ were mixed, 60 ml of toluene, 12 ml of ethanol, and 12 ml of water were added thereto, 50 mg of Pd(OAc)₂ and 230 mg of Xphos were further added thereto, and the mixture was stirred and heated for 4 hours. After the reaction was completed, the temperature was lowered to room temperature, and the reaction product was filtered. The filtrate was poured into water, followed by extraction with chloroform. An organic layer was dried over MgSO₄ and concentrated under reduced pressure, and column chromatography with MC was performed, thereby obtaining 2.0 g (yield 66%) of Compound 376 in a white solid state.

Mass: [(M+H)+]: 654

### [Synthesis Example 15] Synthesis of Compound 377

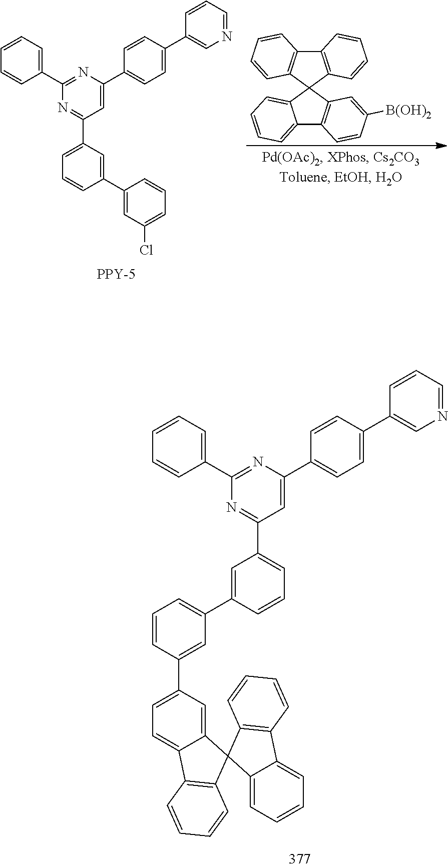

2.0 g of PPY-5, 2.5 g of (9,9-dimethyl-9H-fluoren-2-yl)boronic acid, and 3.0 g of Cs₂CO₃ were mixed, 60 ml of toluene, 12 ml of ethanol, and 12 ml of water were added thereto, 50 mg of Pd(OAc)₂ and 230 mg of Xphos were further added thereto, and the mixture was stirred and heated for 4 hours. After the reaction was completed, the temperature was lowered to room temperature, and the reaction product was filtered. The filtrate was poured into water, followed by extraction with chloroform. An organic layer was dried over MgSO₄ and concentrated under reduced pressure, and column chromatography with THF:Hex=1:2 was performed, thereby obtaining 2.3 g (yield 66%) of Compound 377 in a white solid state.

Mass: [(M+H)+]: 776

### [Synthesis Example 16] Synthesis of Compound 380

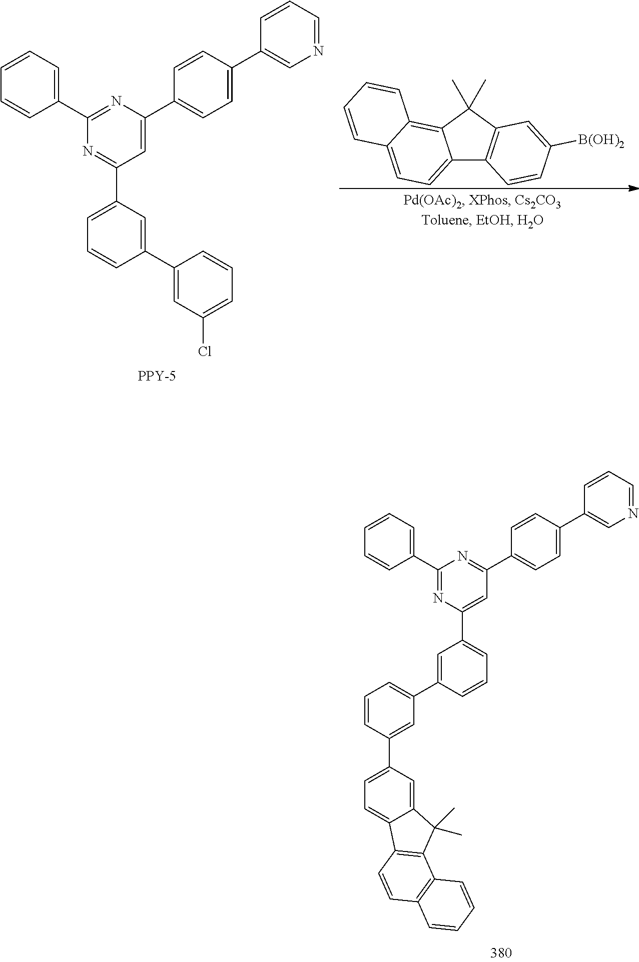 [SMILES: Clc1cccc(-c2cccc(-c3cc(-c4ccc(-c5cccnc5)cc4)nc(-c4ccccc4)n3)c2)c1 ; CC1(C)c2cc(B(O)O)ccc2-c2ccc3ccccc3c21 ; "PPY-5" ; CC1(C)c2cc(-c3cccc(-c4cccc(-c5cc(-c6ccc(-c7cccnc7)cc6)nc(-c6ccccc6)n5)c4)c3)ccc2-c2ccc3ccccc3c21]

2.1 g of PPY-5, 2.4 g of (11,11-dimethyl-11H-benzo[a]fluoren-9-yl)boronic acid, and 2.9 g of Cs₂CO₃ were mixed, 60 ml of toluene, 12 ml of ethanol, and 12 ml of water were added thereto, 53 mg of Pd(OAc)₂ and 240 mg of Xphos were further added thereto, and the mixture was stirred and heated for 4 hours. After the reaction was completed, the temperature was lowered to room temperature, and the reaction product was filtered. The filtrate was poured into water, followed by extraction with chloroform. An organic layer was dried over MgSO₄ and concentrated under reduced pressure, and column chromatography with MC was performed, thereby obtaining 1.9 g (yield 63%) of Compound 380 in a white solid state.

Mass: [(M+H)+]: 704

### [Synthesis Example 17] Synthesis of Compound 409

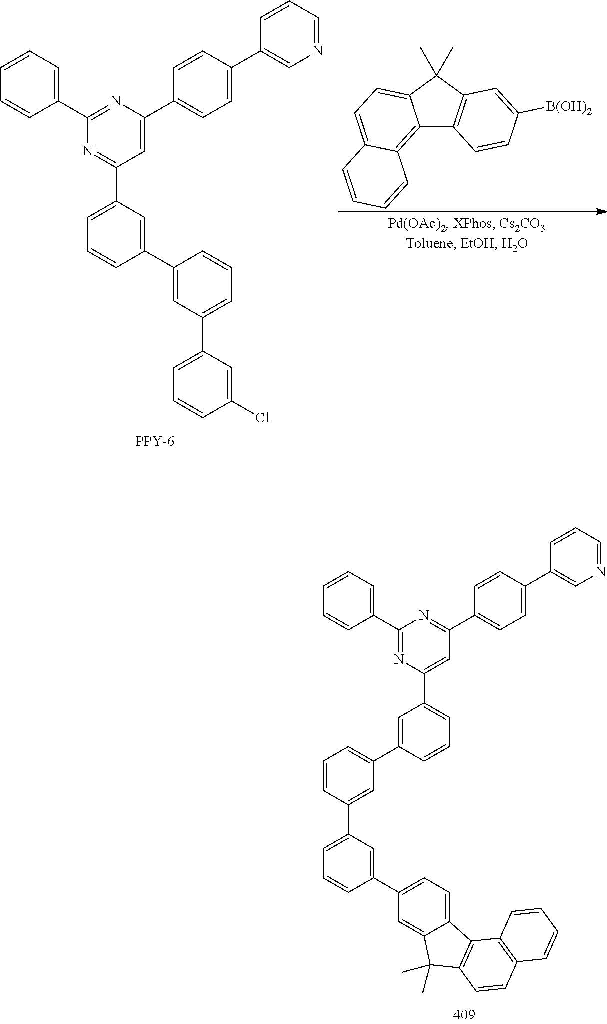 [SMILES: Clc1cccc(-c2cccc(-c3cccc(-c4cc(-c5ccc(-c6cccnc6)cc5)nc(-c5ccccc5)n4)c3)c2)c1 ; CC1(C)c2cc(B(O)O)ccc2-c2c1ccc1ccccc21 ; "PPY-6" ; CC1(C)c2cc(-c3cccc(-c4cccc(-c5cccc(-c6cc(-c7ccc(-c8cccnc8)cc7)nc(-c7ccccc7)n6)c5)c4)c3)ccc2-c2c1ccc1ccccc21]

2.0 g of PPY-6, 2.1 g of (7,7-dimethyl-7H-benzo[c]fluoren-9-yl)boronic acid, and 2.5 g of Cs₂CO₃ were mixed, 60 ml of toluene, 12 ml of ethanol, and 12 ml of water were added thereto, 48 mg of Pd(OAc)₂ and 210 mg of Xphos were further added thereto, and the mixture was stirred and heated for 4 hours. After the reaction was completed, the temperature was lowered to room temperature, and the reaction product was filtered. The filtrate was poured into water, followed by extraction with chloroform. An organic layer was dried over MgSO₄ and concentrated under reduced pressure, and column chromatography with MC:MeOH=100:1 was performed, thereby obtaining 2.1 g (yield 66%) of Compound 409 in a white solid state.

Mass: [(M+H)+]: 780

### [Synthesis Example 18] Synthesis of Compound 411

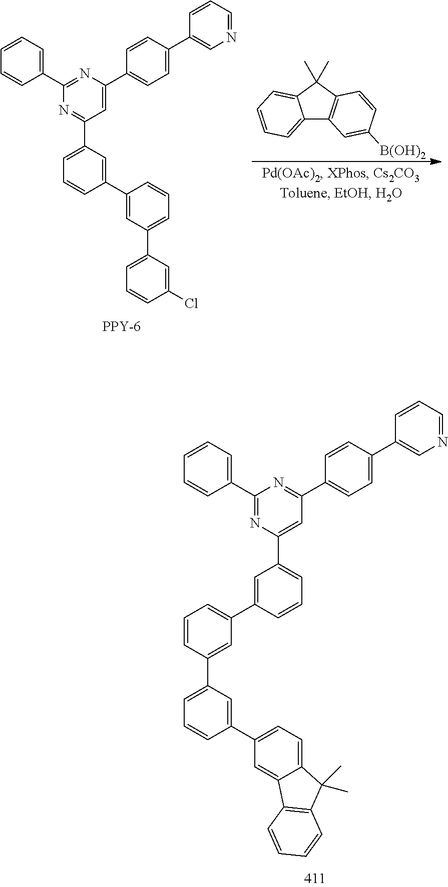 [SMILES: Clc1cccc(-c2cccc(-c3cccc(-c4cc(-c5ccc(-c6cccnc6)cc5)nc(-c5ccccc5)n4)c3)c2)c1 ; CC1(C)c2ccccc2-c2cc(B(O)O)ccc21 ; "PPY-6" ; CC1(C)c2ccccc2-c2cc(-c3cccc(-c4cccc(-c5cccc(-c6cc(-c7ccc(-c8cccnc8)cc7)nc(-c7ccccc7)n6)c5)c4)c3)ccc21]

2.0 g of PPY-6, 2.0 g of (9,9-dimethyl-9H-fluoren-3-yl)boronic acid, and 2.5 g of Cs₂CO₃ were mixed, 60 ml of toluene, 12 ml of ethanol, and 12 ml of water were added thereto, 48 mg of Pd(OAc)₂ and 210 mg of Xphos were further added thereto, and the mixture was stirred and heated for 4 hours. After the reaction was completed, the temperature was lowered to room temperature, and the reaction product was filtered. The filtrate was poured into water, followed by extraction with chloroform. An organic layer was dried over MgSO₄ and concentrated under reduced pressure, and column chromatography with MC:MeOH=100:1 was performed, thereby obtaining 1.6 g (yield 59%) of Compound 411 in a white solid state.

Mass: [(M+H)+]: 730

### [Synthesis Example 19] Synthesis of Compound 436

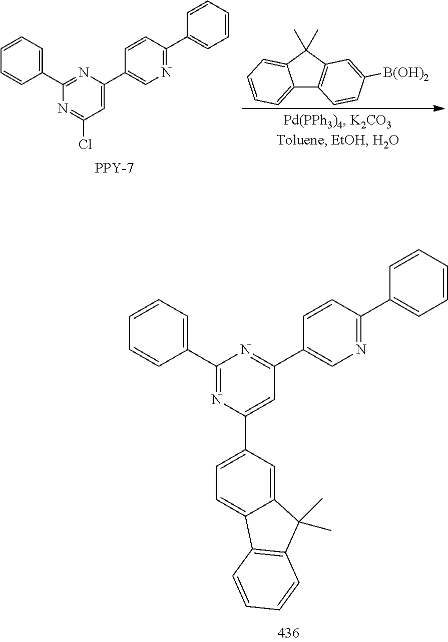

3.0 g of PPY-7, 4.6 g of (9,9′-dimethyl-9H-fluoren-2-yl)boronic acid, and 3.2 g of K₂CO₃ were mixed, 60 ml of toluene, 12 ml of ethanol, and 12 ml of water were added thereto, 500 mg of tetrakis(phenylphosphine)palladium (0) was further added thereto, and the mixture was stirred and heated for 4 hours. After the reaction was completed, the temperature was lowered to room temperature, and the reaction product was filtered. The filtrate was poured into water, followed by extraction with chloroform. An organic layer was dried over MgSO₄ and concentrated under reduced pressure, and column chromatography with MC:Hex=2:1 was performed, thereby obtaining 3.0 g (yield 65%) of Compound 436 in a white solid state.

Mass: [(M+H)+]: 502

### [Synthesis Example 20] Synthesis of Compound 448

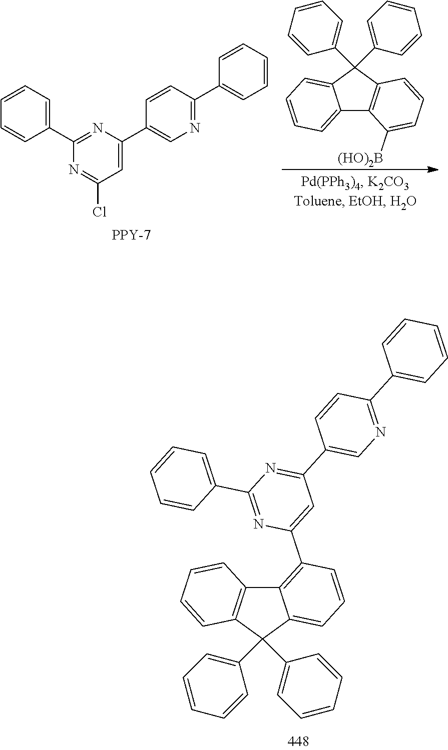

2.9 g of PPY-7, 5.0 g of (9,9-diphenyl-9H-fluoren-4-yl)boronic acid, and 3.1 g of K₂CO₃ were mixed, 60 ml of toluene, 12 ml of ethanol, and 12 ml of water were added thereto, 500 mg of tetrakis(phenylphosphine)palladium (0) was further added thereto, and the mixture was stirred and heated for 4 hours. After the reaction was completed, the temperature was lowered to room temperature, and the reaction product was filtered. The filtrate was poured into water, followed by extraction with chloroform. An organic layer was dried over MgSO₄ and concentrated under reduced pressure, and column chromatography with MC:Hex=2:1 was performed, thereby obtaining 3.9 g (yield 62%) of Compound 448 in a white solid state.

Mass: [(M+H)+]: 626

### [Synthesis Example 21] Synthesis of Compound 518

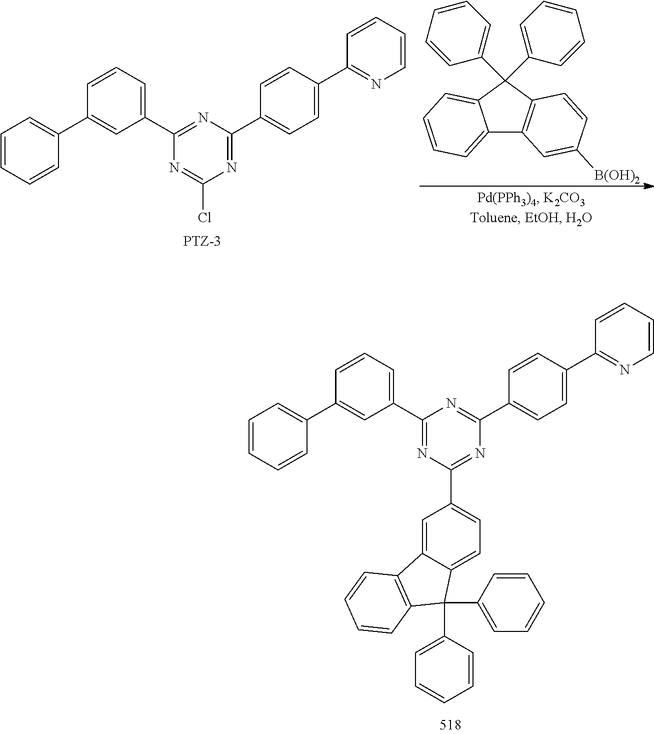

2.6 g of PTZ-3, 4.6 g of (9,9′-diphenyl-9H-fluoren-3-yl)boronic acid, and 3.3 g of K₂CO₃ were mixed, 60 ml of toluene, 12 ml of ethanol, and 12 ml of water were added thereto, 480 mg of tetrakis(phenylphosphine)palladium (0) was further added thereto, and the mixture was stirred and heated for 4 hours. After the reaction was completed, the temperature was lowered to room temperature, and the reaction product was filtered. The filtrate was poured into water, followed by extraction with chloroform. An organic layer was dried over MgSO₄ and concentrated under reduced pressure, and column chromatography with MC:Hex=2:1 was performed, thereby obtaining 4.2 g (yield 72%) of Compound 518 in a white solid state.

Mass: [(M+H)+]: 703

### [Synthesis Example 22] Synthesis of Compound 524

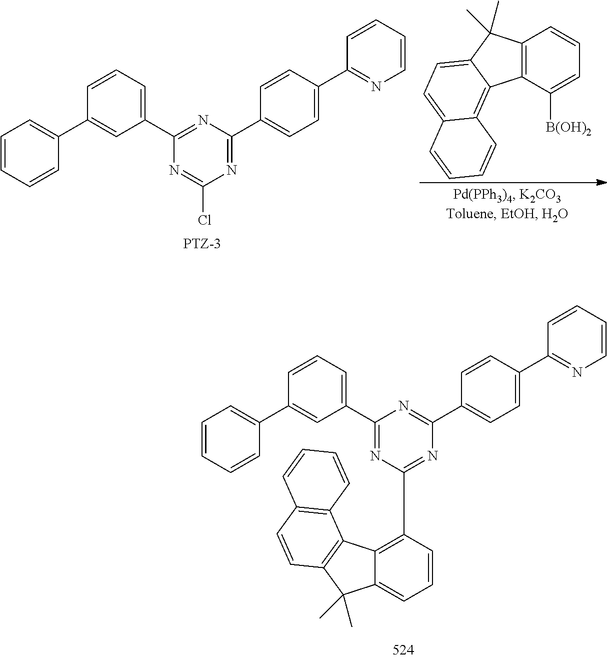

2.0 g of PTZ-3, 3.6 g of (7,7-dimethyl-7H-benzo[c]fluoren-11-yl)boronic acid, and 2.3 g of K₂CO₃ were mixed, 50 ml of toluene, 10 ml of ethanol, and 10 ml of water were added thereto, 400 mg of tetrakis(phenylphosphine)palladium (0) was further added thereto, and the mixture was stirred and heated for 4 hours. After the reaction was completed, the temperature was lowered to room temperature, and the reaction product was filtered. The filtrate was poured into water, followed by extraction with chloroform. An organic layer was dried over MgSO₄ and concentrated under reduced pressure, and column chromatography with MC:Hex=2:1 was performed, thereby obtaining 4.2 g (yield 72%) of Compound 524 in a white solid state.

Mass: [(M+H)+]: 629

### [Synthesis Example 23] Synthesis of Compound 542

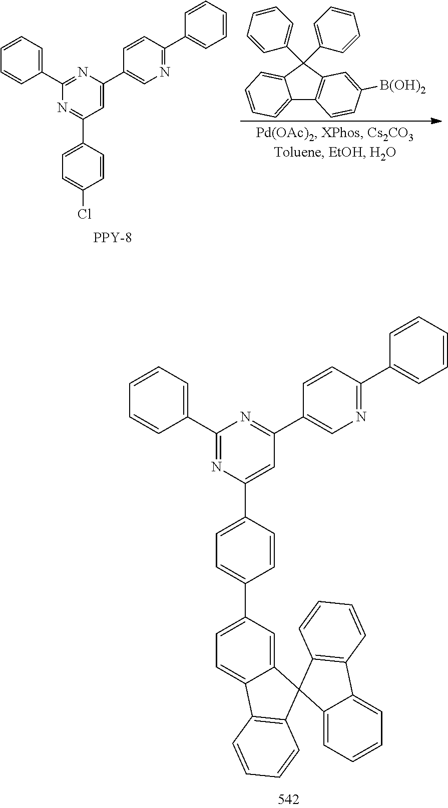

2.2 g of PPY-8, 2.6 g of 9,9′-spirobi[fluorene]-2-yl boronic acid, and 2.9 g of Cs₂CO₃ were mixed, 60 ml of toluene, 12 ml of ethanol, and 12 ml of water were added thereto, 50 mg of Pd(OAc)₂ and 230 mg of Xphos were further added thereto, and the mixture was stirred and heated for 4 hours. After the reaction was completed, the temperature was lowered to room temperature, and the reaction product was filtered. The filtrate was poured into water, followed by extraction with chloroform. An organic layer was dried over MgSO₄ and concentrated under reduced pressure, and column chromatography with MC was performed, thereby obtaining 2.1 g (yield 53%) of Compound 542 in a white solid state.

Mass: [(M+H)+]: 700

### [Synthesis Example 24] Synthesis of Compound 545

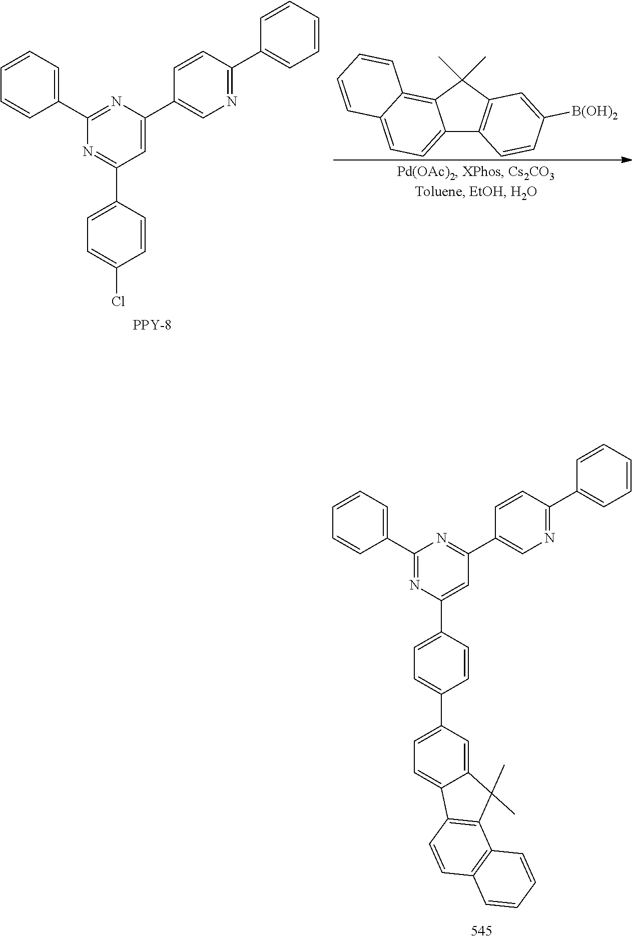

2.3 g of PPY-8, 2.4 g of (11,11-dimethyl-11H-benzo[a]fluoren-9-yl)boronic acid, and 3.0 g of Cs₂CO₃ were mixed, 60 ml of toluene, 12 ml of ethanol, and 12 ml of water were added thereto, 55 mg of Pd(OAc)₂ and 250 mg of Xphos were further added thereto, and the mixture was stirred and heated for 4 hours. After the reaction was completed, the temperature was lowered to room temperature, and the reaction product was filtered. The filtrate was poured into water, followed by extraction with chloroform. An organic layer was dried over MgSO₄ and concentrated under reduced pressure, and column chromatography with THF:Hex=1:3 was performed, thereby obtaining 2.6 g (yield 63%) of Compound 545 in a white solid state.

Mass: [(M+H)+]: 628

### [Embodiments 1 to 13] Manufacturing of Blue Organic Electroluminescent Device

Compounds 1, 2, 4, 42, 45, 111, 112, 121, 133, 151, 156, 346, and 350 synthesized in the above Synthesis Examples were subjected to high purity sublimation purification by a commonly known method and then blue organic EL devices were manufactured as follows.

First, a glass substrate thin-film-coated with indium tin oxide (ITO) to a thickness of 1500 Å was washed with distilled water ultrasonically. After washing with distilled water was completed, the glass substrate was ultrasonically cleaned with a solvent, such as isopropyl alcohol, acetone and methanol, dried, transferred to a UV OZONE cleaner (Power sonic 405, Hwasin Tech) cleaned for 5 minutes using UV, and then transferred to a vacuum evaporator.

On the ITO transparent electrode prepared as above, DS-205 (Doosan Electronics CO., LTD., 80 nm)/NPB (15 nm)/ADN+5% DS-405 (Doosan Electronics CO., LTD., 30 nm)/respective Compounds 1, 2, 4, 42, 45, 111, 112, 121, 133, 151, 156, 346, and 350 (30 nm)/LiF (1 nm)/Al (200 nm) were laminated in the order listed, thereby manufacturing organic EL devices.

### [Comparative Example 1] Manufacturing of Blue Organic EL Device

A blue organic EL device was manufactured in the same manner as in Embodiment 1, except that Alq₃, instead of Compound 1, was used as the material of the electron transporting layer.

### [Comparative Example 2] Manufacturing of Blue Organic EL Device

A blue organic EL device was manufactured in the same manner as in Embodiment 1, except that Compound 1 was not used as the material of the electron transporting layer.

The structures of NPB, ADN, and Alq₃ used in Embodiments 1 to 13 and Comparative Examples 1 and 2 are as follows.

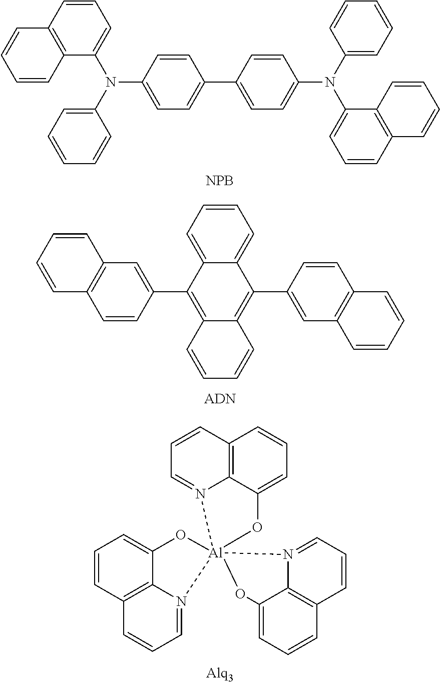

### Evaluation Example 1

For each of the blue organic EL devices manufactured in Embodiments 1 to 13 and Comparative Examples 1 and 2, a driving voltage, a current efficiency and a light emission peak at a current density of 10 mA/cm² were measured and the results are shown in Table 1 below.

TABLE 1

| | Electron | Driving | Emission | Current |
|---|---|---|---|---|
| | transporting | voltage | peak | efficiency |
| Sample | layer | (V) | (nm) | (cd/A) |
| Embodiment 1 | Compound 1 | 3.6 | 455 | 8.1 |
| Embodiment 2 | Compound 2 | 3.8 | 451 | 8.6 |
| Embodiment 3 | Compound 4 | 3.8 | 452 | 9.1 |
| Embodiment 4 | Compound 42 | 3.6 | 452 | 8.5 |
| Embodiment 5 | Compound 45 | 3.7 | 453 | 8.6 |
| Embodiment 6 | Compound 111 | 3.6 | 451 | 8.8 |
| Embodiment 7 | Compound 112 | 3.9 | 451 | 9.1 |
| Embodiment 8 | Compound 121 | 3.4 | 453 | 7.7 |
| Embodiment 9 | Compound 133 | 3.3 | 452 | 7.6 |
| Embodiment 10 | Compound 151 | 3.1 | 451 | 7.1 |
| Embodiment 11 | Compound 156 | 3.2 | 450 | 7.3 |
| Embodiment 12 | Compound 346 | 4.3 | 451 | 8.9 |
| Embodiment 13 | Compound 350 | 4.4 | 453 | 9.0 |
| Comparative | Alq₃ | 4.8 | 457 | 5.6 |
| example 1 | | | | |
| Comparative | — | 4.7 | 459 | 6.1 |
| example 2 | | | | |

As shown in Table 1, it was appreciated that the blue organic EL devices (Embodiments 1 to 13) in which Compounds 11, 2, 4, 42, 45, 111, 112, 121, 133, 151, 156, 346 and 350 of the present disclosure, synthesized in the above Synthesis Examples, were used in the electron transporting layer exhibited excellent performance in terms of the driving voltage, the emission peak and the current efficiency, as compared with a conventional blue organic EL device (Comparative Example 1) in which Alq₃ was used in the electron transporting layer and a conventional blue organic EL device (Comparative Example 2) in which the electron transporting layer is absent.

### [Embodiments 14 to 24] Manufacturing of Blue Organic Electroluminescent Device

Compounds 376, 377, 380, 409, 411, 436, 448, 518, 524, 542, and 545 synthesized in the above Synthesis Examples were subjected to high purity sublimation purification by a commonly known method and then blue organic EL devices were manufactured as follows.

First, a glass substrate thin-film-coated with indium tin oxide (ITO) to a thickness of 1500 Å was washed with distilled water ultrasonically. After washing with distilled water was completed, the glass substrate was ultrasonically cleaned with a solvent, such as isopropyl alcohol, acetone and methanol, dried, transferred to a UV OZONE cleaner (Power sonic 405, Hwasin Tech) cleaned for 5 minutes using UV, and then transferred to a vacuum evaporator.

On the ITO transparent electrode prepared as above, DS-205 (Doosan Electronics CO., LTD., 80 nm)/NPB (15 nm)/ADN+5% DS-405 (Doosan Electronics CO., LTD., 30 nm)/respective Compounds 376, 377, 380, 409, 411, 436, 448, 518, 524, 542, and 545 (5 nm)/Alq₃ (25 nm)/LiF (1 nm)/Al (200 nm) were laminated in the order listed, thereby manufacturing organic EL devices.

### [Comparative Example 3] Manufacturing of Blue Organic EL Device

A blue organic EL device was manufactured in the same manner as in Embodiment 14, except that Compound 376 was not used as a material of an electron transport auxiliary layer and that Alq₃, which is a material for the electron transporting layer, was laminated to 30 nm rather than 25 nm.

### Evaluation Example 2

For each of the blue organic EL devices manufactured in Embodiments 14 to 24 and Comparative Example 3, a driving voltage, a current efficiency and a light emission peak at a current density of 10 mA/cm² were measured and the results are shown in Table 2 below.

TABLE 2

| | Electron | Driving | Emission | Current |
|---|---|---|---|---|
| | transporting | voltage | peak | efficiency |
| Sample | auxiliary layer | (V) | (nm) | (cd/A) |
| Embodiment 14 | Compound 376 | 3.7 | 456 | 9.0 |
| Embodiment 15 | Compound 377 | 3.6 | 455 | 8.8 |
| Embodiment 16 | Compound 380 | 3.5 | 456 | 8.6 |
| Embodiment 17 | Compound 409 | 3.9 | 455 | 8.5 |
| Embodiment 18 | Compound 411 | 3.4 | 456 | 9.1 |
| Embodiment 19 | Compound 436 | 3.3 | 457 | 8.8 |
| Embodiment 20 | Compound 448 | 3.6 | 455 | 9.1 |
| Embodiment 21 | Compound 518 | 3.4 | 454 | 8.4 |
| Embodiment 22 | Compound 524 | 3.7 | 455 | 8.6 |
| Embodiment 23 | Compound 542 | 3.4 | 456 | 8.8 |
| Embodiment 24 | Compound 545 | 3.6 | 455 | 9.3 |
| Comparative | — | 4.7 | 459 | 6.1 |
| example 3 | | | | |

As shown in Table 2, it was appreciated that the blue organic EL devices (Embodiments 14 to 24) in which the compounds of the present disclosure, synthesized in the above Synthesis Examples, were used in the electron transporting auxiliary layer exhibited excellent performance in terms of the driving voltage, the emission peak and the current efficiency, as compared with conventional blue organic EL device (Comparative Example 3) in which the electron transporting auxiliary layer is absent.

Although the preferred embodiments of the present disclosure have been described above, the present disclosure is not limited thereto, and various modifications and changes may be made within the scope of the claims and the detailed description of the invention, which also fall within the scope of the invention.

1. A compound represented by the following Chemical Formula 1: 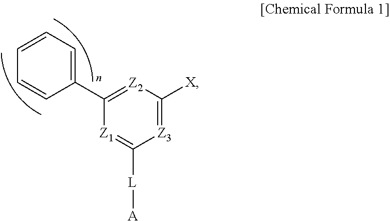
where in Chemical Formula 1,
Z₁ to Z₃ are each independently nitrogen, and
X is represented by the following Chemical Formula 2 or Chemical Formula 3, 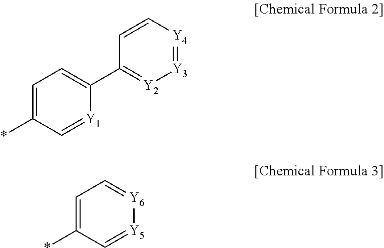
in Chemical Formula 2 and Chemical Formula 3,
one of Y₁ to Y₄ is nitrogen and the others are carbons, and one of Y₅ and Y₆ is nitrogen and the other is carbon,
* means a site where a bond with Chemical Formula 1 is made,
n is an integer ranging from 1 to 3,
L is a single bond, or selected from the group consisting of a C₆ to C₁₈ arylene group and a heteroarylene group having 5 to 18 nuclear atoms, and
A is represented by the following Chemical Formula 4, and 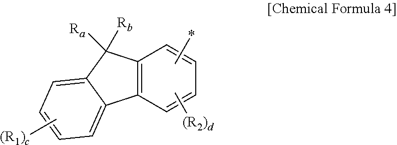
in Chemical Formula 4,
Rₐ and R_{b} are the same as or different from each other, each independently a C₁ to C₄₀ alkyl group or C₆ to C₆₀ aryl group, or bound with each other to form a fused ring,
R₁ and R₂ are the same as or different from each other, each independently selected from the group consisting of: hydrogen, deuterium, a halogen group, a cyano group, a nitro group, an amino group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkyl boron group, a C₆ to C₆₀ aryl boron group, a C₁ to C₄₀ phosphine group, a C₁ to C₄₀ phosphine oxide group, and a C₆ to C₆₀ arylamine group, or bound with an adjacent group to form a fused ring,
c is an integer ranging from 0 to 4,
d is an integer ranging from 0 to 3,
* means a site where a bond with Chemical Formula 1 is made,
the alkyl group and the aryl group of Rₐ and R_{b}, the alkyl group, the alkenyl group, the alkynyl group, the cycloalkyl group, the heterocycloalkyl group, the aryl group, the heteroaryl group, the alkyloxy group, the aryloxy group, the alkylsilyl group, the arylsilyl group, the alkylboron group, the arylboron group, the phosphine group, the phosphine oxide group, and the arylamine group of R₁ and R₂, and the arylene group and the heteroarylene group of L are each independently substituted or unsubstituted with one or more kinds of substituents selected from the group consisting of: deuterium, a halogen group, a cyano group, a nitro group, an amino group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkyl boron group, a C₆ to C₆₀ arylboron group, a C₁ to C₄₀ phosphine group, a C₁ to C₄₀ phosphine oxide group, and a C₆ to C₆₀ arylamine group, and when the substituents are plural in number, the plurality of substituents are the same as or different from each other.

2. The compound of claim 1, wherein the compound represented by Chemical Formula 1 is a compound represented by any one of the following Chemical Formula 7 and Chemical Formula 10: 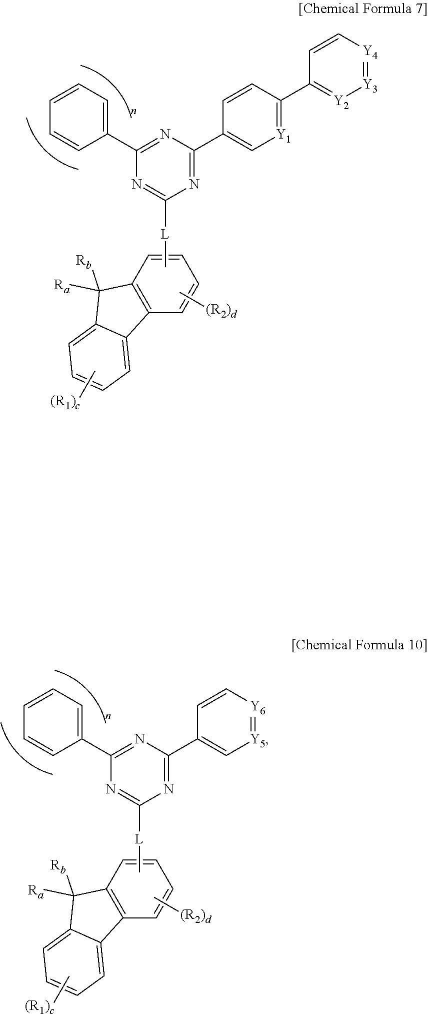
where in Chemical Formula 7 and Chemical Formula 10,
Rₐ, R_{b}, R₁, R₂, Y₁ to Y₆, L, c, d and n are the same as those defined in claim 1, respectively.

3. The compound of claim 1, wherein in Chemical Formula 1, X is selected from the group consisting of the following structures represented by X-1 to X-6:

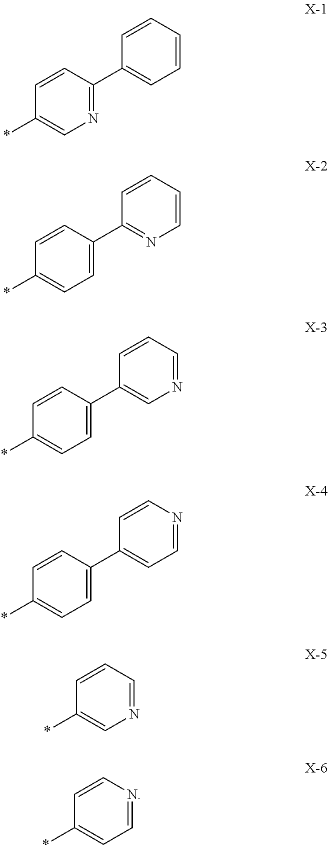 [SMILES: *c1ccc(-c2ccccc2)nc1 ; *c1ccc(-c2ccccn2)cc1 ; *c1ccc(-c2cccnc2)cc1 ; *c1ccc(-c2ccncc2)cc1 ; *c1cccnc1 ; *c1ccncc1]

4. The compound of claim 1, wherein in Chemical Formula 1, a structure represented by 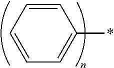
(* being a site where a bond is made) is selected from the group consisting of the following structures represented by Ar-1 to Ar-3:

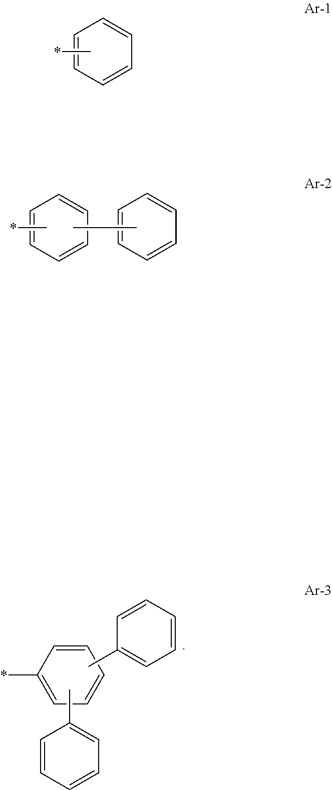

5. The compound of claim 1, wherein Rₐ and R_{b} are each independently a methyl group or a phenyl group, or combined with each other to form a fused ring represented by 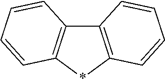 [SMILES: *1c2ccccc2c2ccccc12]
(* being a site where a bond is made).

6. The compound of claim 1, wherein in Chemical Formula 1, A is selected from the group consisting of the following structures represented by A-1 to A-6:

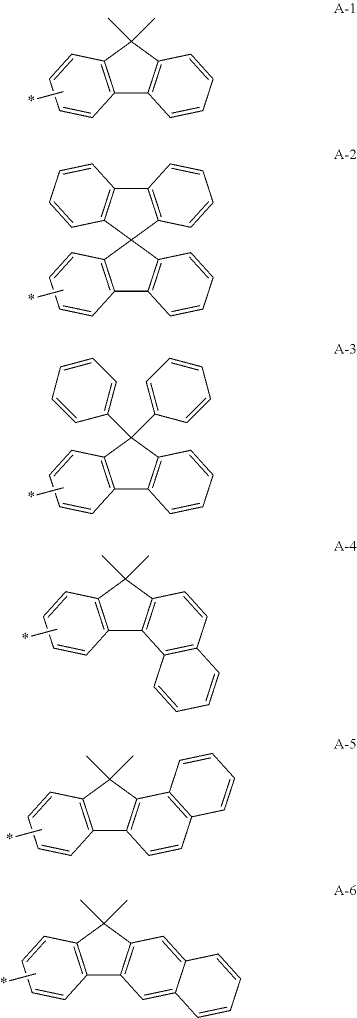

7. The compound of claim 1, wherein in Chemical Formula 1, L is a single bond, or a linking group selected from the following structures represented by L-1 to L-7:

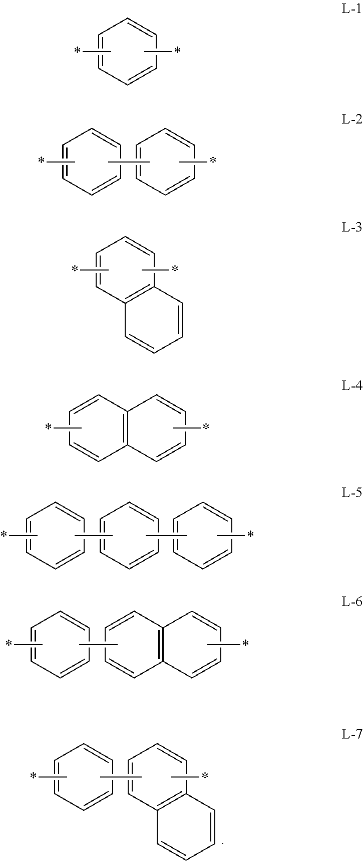

8. The compound of claim 1, wherein the compound represented by Chemical Formula 1 is selected from the group consisting of the following compounds represented by 31-45, 61-90, 136-165, 226-255, 286-315, 331-345, 361-375, 391-405, 421-435, 466-480, 496-525, 571-600, 646-660, 676-690, 706-720, and 736-750:

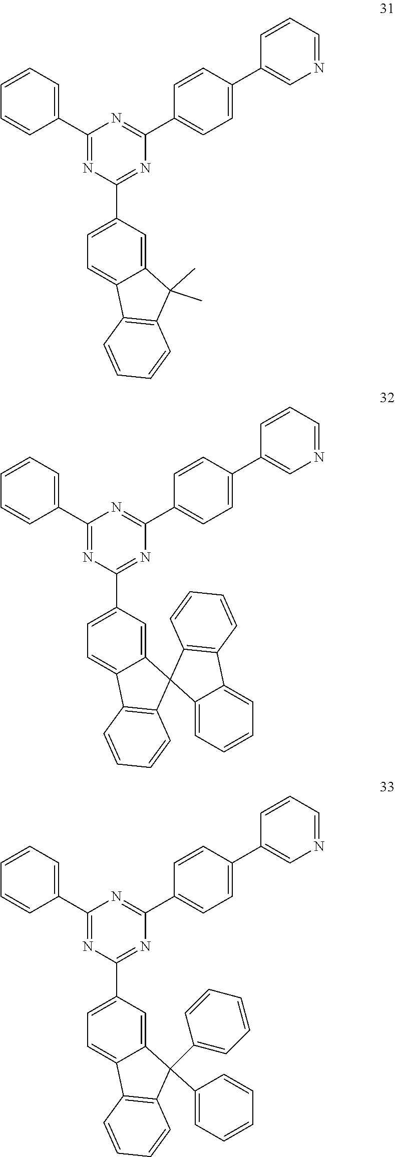 [SMILES: CC1(C)c2ccccc2-c2ccc(-c3nc(-c4ccccc4)nc(-c4ccc(-c5cccnc5)cc4)n3)cc21 ; c1ccc(-c2nc(-c3ccc(-c4cccnc4)cc3)nc(-c3ccc4c(c3)C3(c5ccccc5-c5ccccc53)c3ccccc3-4)n2)cc1 ; c1ccc(-c2nc(-c3ccc(-c4cccnc4)cc3)nc(-c3ccc4c(c3)C(c3ccccc3)(c3ccccc3)c3ccccc3-4)n2)cc1]

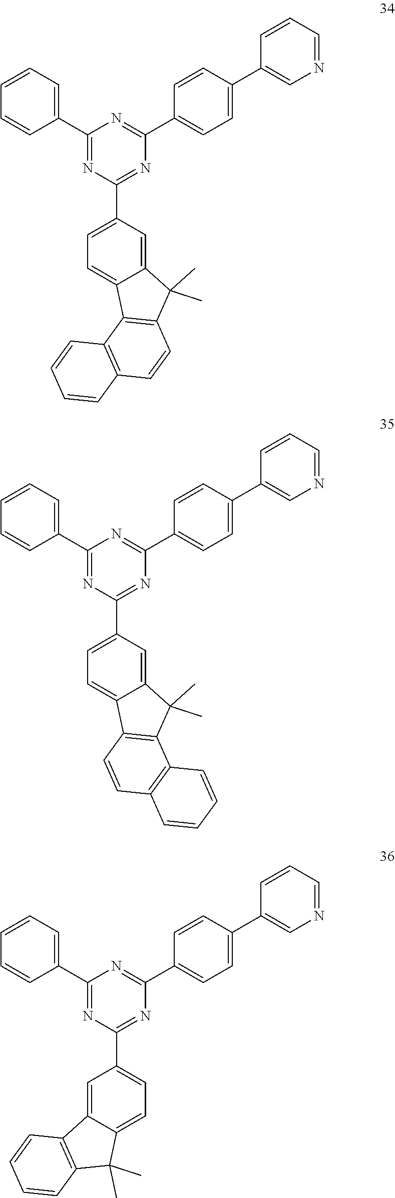 [SMILES: CC1(C)c2cc(-c3nc(-c4ccccc4)nc(-c4ccc(-c5cccnc5)cc4)n3)ccc2-c2c1ccc1ccccc21 ; CC1(C)c2cc(-c3nc(-c4ccccc4)nc(-c4ccc(-c5cccnc5)cc4)n3)ccc2-c2ccc3ccccc3c21 ; CC1(C)c2ccccc2-c2cc(-c3nc(-c4ccccc4)nc(-c4ccc(-c5cccnc5)cc4)n3)ccc21]

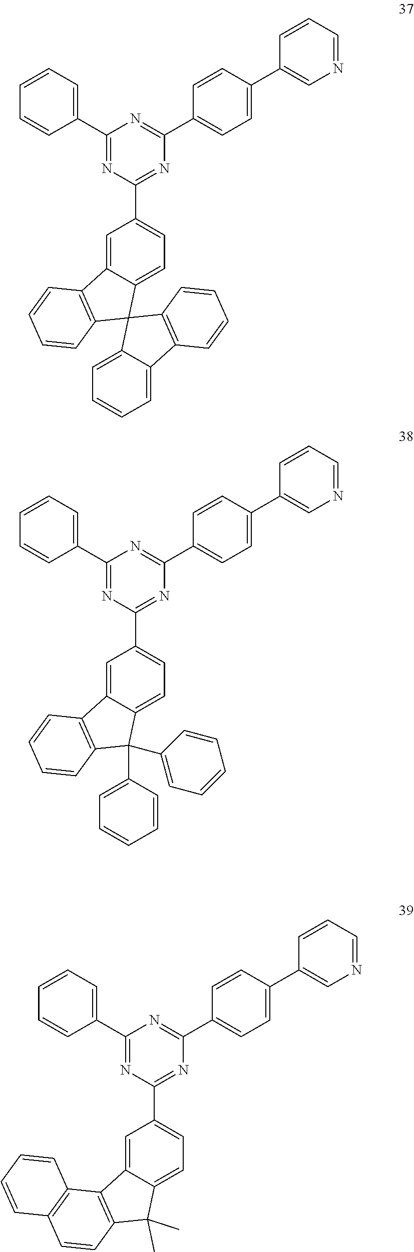 [SMILES: c1ccc(-c2nc(-c3ccc(-c4cccnc4)cc3)nc(-c3ccc4c(c3)-c3ccccc3C43c4ccccc4-c4ccccc43)n2)cc1 ; c1ccc(-c2nc(-c3ccc(-c4cccnc4)cc3)nc(-c3ccc4c(c3)-c3ccccc3C4(c3ccccc3)c3ccccc3)n2)cc1 ; CC1(C)c2ccc(-c3nc(-c4ccccc4)nc(-c4ccc(-c5cccnc5)cc4)n3)cc2-c2c1ccc1ccccc21]

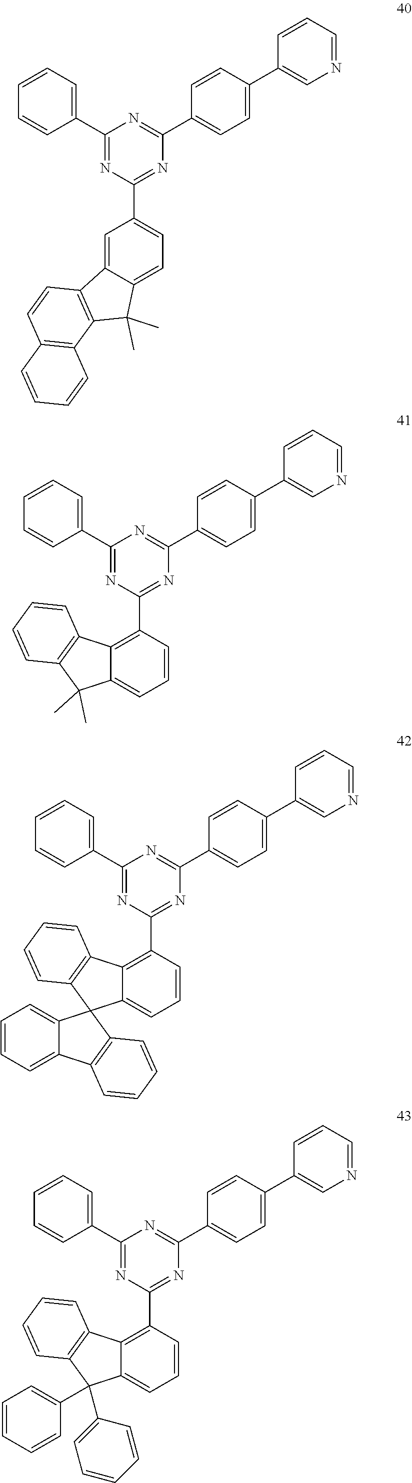 [SMILES: CC1(C)c2ccc(-c3nc(-c4ccccc4)nc(-c4ccc(-c5cccnc5)cc4)n3)cc2-c2ccc3ccccc3c21 ; CC1(C)c2ccccc2-c2c(-c3nc(-c4ccccc4)nc(-c4ccc(-c5cccnc5)cc4)n3)cccc21 ; c1ccc(-c2nc(-c3ccc(-c4cccnc4)cc3)nc(-c3cccc4c3-c3ccccc3C43c4ccccc4-c4ccccc43)n2)cc1 ; c1ccc(-c2nc(-c3ccc(-c4cccnc4)cc3)nc(-c3cccc4c3-c3ccccc3C4(c3ccccc3)c3ccccc3)n2)cc1]

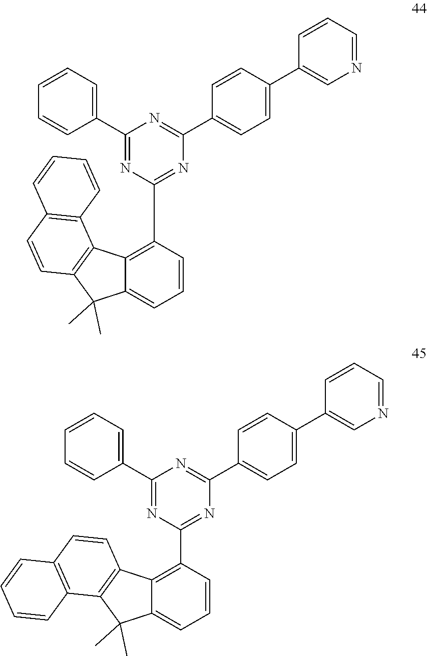 [SMILES: CC1(C)c2cccc(-c3nc(-c4ccccc4)nc(-c4ccc(-c5cccnc5)cc4)n3)c2-c2c1ccc1ccccc21 ; CC1(C)c2cccc(-c3nc(-c4ccccc4)nc(-c4ccc(-c5cccnc5)cc4)n3)c2-c2ccc3ccccc3c21]

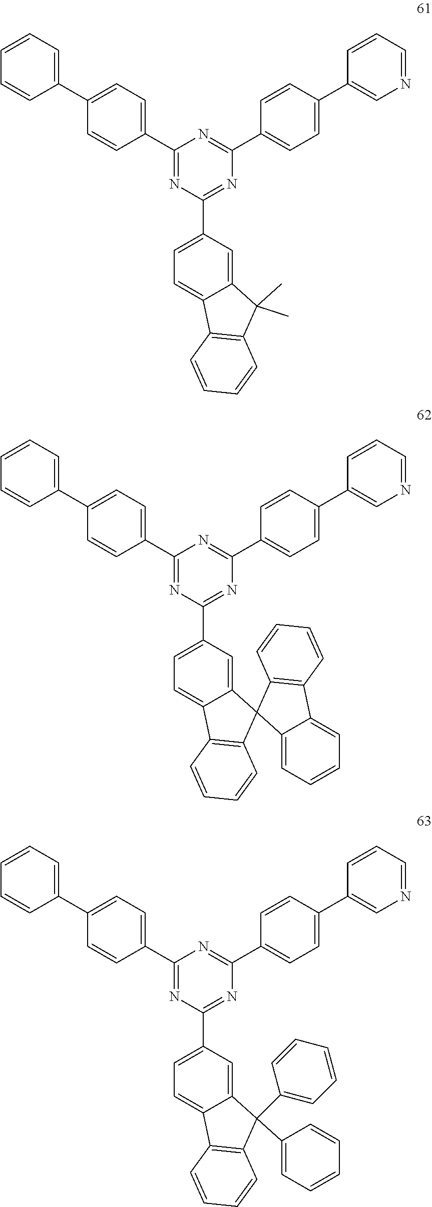 [SMILES: CC1(C)c2ccccc2-c2ccc(-c3nc(-c4ccc(-c5ccccc5)cc4)nc(-c4ccc(-c5cccnc5)cc4)n3)cc21 ; c1ccc(-c2ccc(-c3nc(-c4ccc(-c5cccnc5)cc4)nc(-c4ccc5c(c4)C4(c6ccccc6-c6ccccc64)c4ccccc4-5)n3)cc2)cc1 ; c1ccc(-c2ccc(-c3nc(-c4ccc(-c5cccnc5)cc4)nc(-c4ccc5c(c4)C(c4ccccc4)(c4ccccc4)c4ccccc4-5)n3)cc2)cc1]

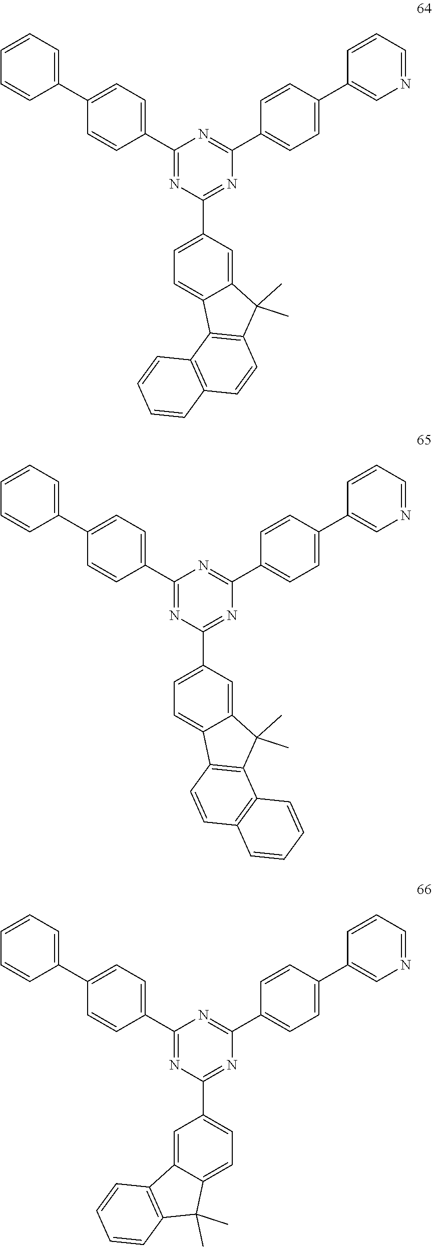 [SMILES: CC1(C)c2cc(-c3nc(-c4ccc(-c5ccccc5)cc4)nc(-c4ccc(-c5cccnc5)cc4)n3)ccc2-c2c1ccc1ccccc21 ; CC1(C)c2cc(-c3nc(-c4ccc(-c5ccccc5)cc4)nc(-c4ccc(-c5cccnc5)cc4)n3)ccc2-c2ccc3ccccc3c21 ; CC1(C)c2ccccc2-c2cc(-c3nc(-c4ccc(-c5ccccc5)cc4)nc(-c4ccc(-c5cccnc5)cc4)n3)ccc21]

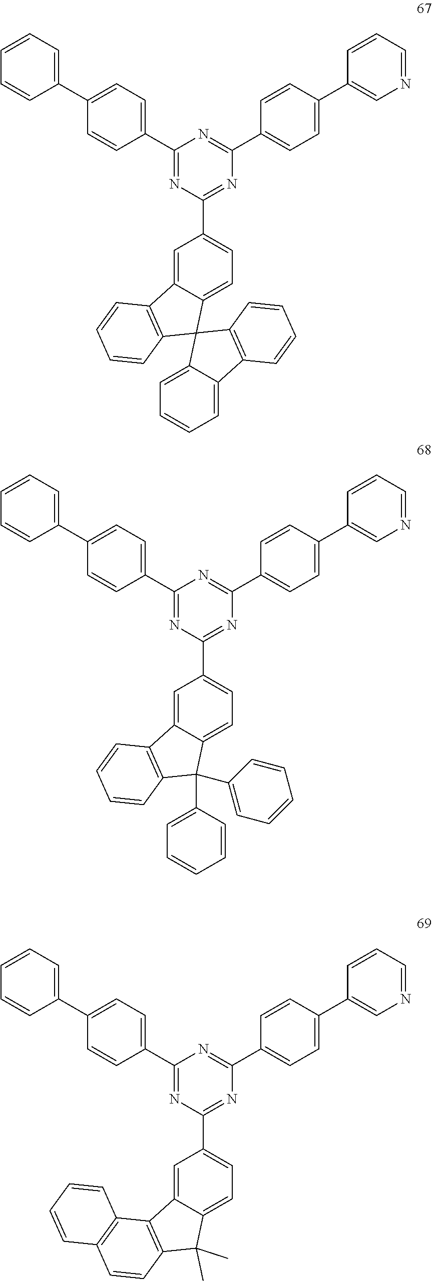 [SMILES: c1ccc(-c2ccc(-c3nc(-c4ccc(-c5cccnc5)cc4)nc(-c4ccc5c(c4)-c4ccccc4C54c5ccccc5-c5ccccc54)n3)cc2)cc1 ; c1ccc(-c2ccc(-c3nc(-c4ccc(-c5cccnc5)cc4)nc(-c4ccc5c(c4)-c4ccccc4C5(c4ccccc4)c4ccccc4)n3)cc2)cc1 ; CC1(C)c2ccc(-c3nc(-c4ccc(-c5ccccc5)cc4)nc(-c4ccc(-c5cccnc5)cc4)n3)cc2-c2c1ccc1ccccc21]

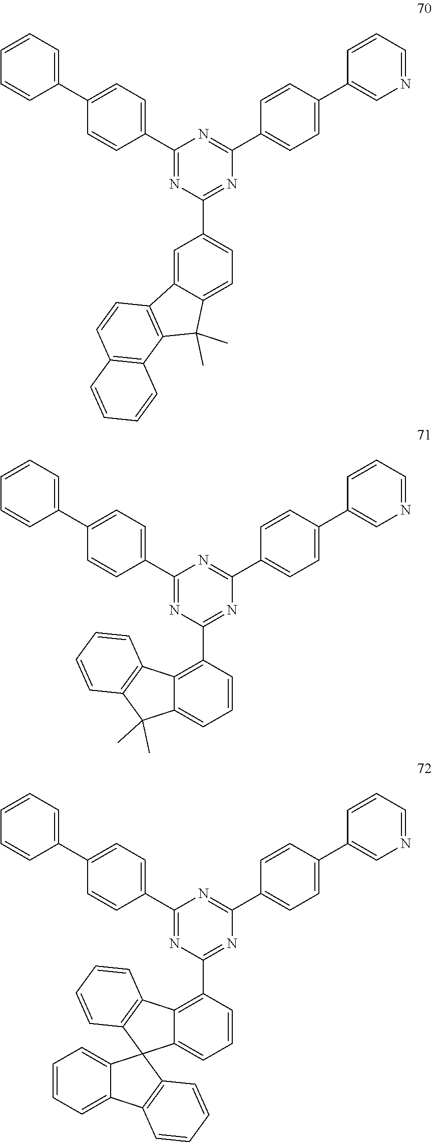 [SMILES: CC1(C)c2ccc(-c3nc(-c4ccc(-c5ccccc5)cc4)nc(-c4ccc(-c5cccnc5)cc4)n3)cc2-c2ccc3ccccc3c21 ; CC1(C)c2ccccc2-c2c(-c3nc(-c4ccc(-c5ccccc5)cc4)nc(-c4ccc(-c5cccnc5)cc4)n3)cccc21 ; c1ccc(-c2ccc(-c3nc(-c4ccc(-c5cccnc5)cc4)nc(-c4cccc5c4-c4ccccc4C54c5ccccc5-c5ccccc54)n3)cc2)cc1]

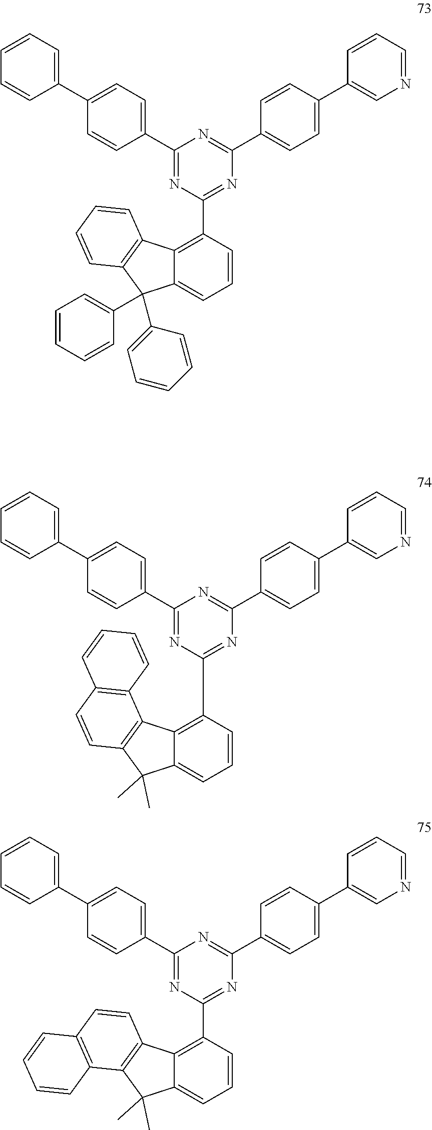 [SMILES: c1ccc(-c2ccc(-c3nc(-c4ccc(-c5cccnc5)cc4)nc(-c4cccc5c4-c4ccccc4C5(c4ccccc4)c4ccccc4)n3)cc2)cc1 ; CC1(C)c2cccc(-c3nc(-c4ccc(-c5ccccc5)cc4)nc(-c4ccc(-c5cccnc5)cc4)n3)c2-c2c1ccc1ccccc21 ; CC1(C)c2cccc(-c3nc(-c4ccc(-c5ccccc5)cc4)nc(-c4ccc(-c5cccnc5)cc4)n3)c2-c2ccc3ccccc3c21]

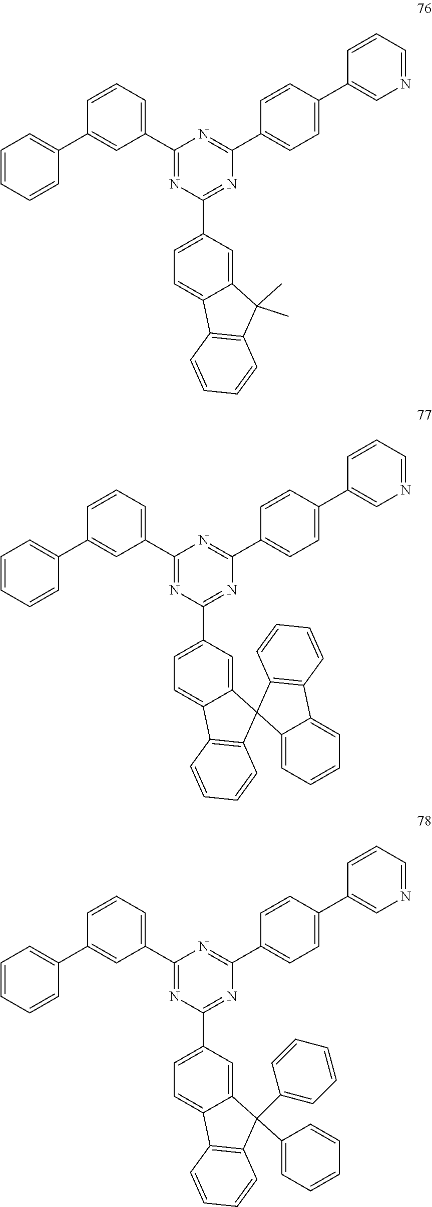 [SMILES: CC1(C)c2ccccc2-c2ccc(-c3nc(-c4ccc(-c5cccnc5)cc4)nc(-c4cccc(-c5ccccc5)c4)n3)cc21 ; c1ccc(-c2cccc(-c3nc(-c4ccc(-c5cccnc5)cc4)nc(-c4ccc5c(c4)C4(c6ccccc6-c6ccccc64)c4ccccc4-5)n3)c2)cc1 ; c1ccc(-c2cccc(-c3nc(-c4ccc(-c5cccnc5)cc4)nc(-c4ccc5c(c4)C(c4ccccc4)(c4ccccc4)c4ccccc4-5)n3)c2)cc1]

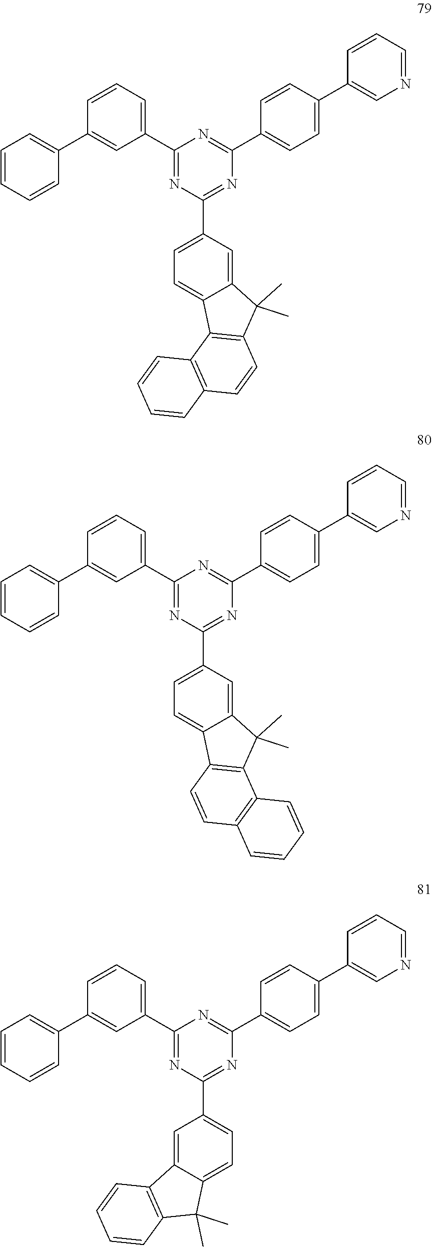 [SMILES: CC1(C)c2cc(-c3nc(-c4ccc(-c5cccnc5)cc4)nc(-c4cccc(-c5ccccc5)c4)n3)ccc2-c2c1ccc1ccccc21 ; CC1(C)c2cc(-c3nc(-c4ccc(-c5cccnc5)cc4)nc(-c4cccc(-c5ccccc5)c4)n3)ccc2-c2ccc3ccccc3c21 ; CC1(C)c2ccccc2-c2cc(-c3nc(-c4ccc(-c5cccnc5)cc4)nc(-c4cccc(-c5ccccc5)c4)n3)ccc21]

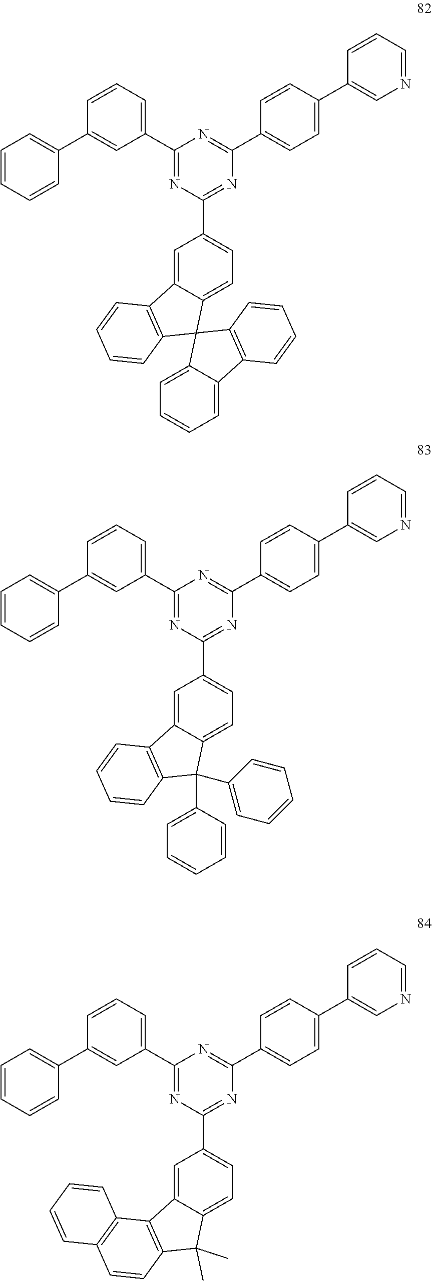 [SMILES: c1ccc(-c2cccc(-c3nc(-c4ccc(-c5cccnc5)cc4)nc(-c4ccc5c(c4)-c4ccccc4C54c5ccccc5-c5ccccc54)n3)c2)cc1 ; c1ccc(-c2cccc(-c3nc(-c4ccc(-c5cccnc5)cc4)nc(-c4ccc5c(c4)-c4ccccc4C5(c4ccccc4)c4ccccc4)n3)c2)cc1 ; CC1(C)c2ccc(-c3nc(-c4ccc(-c5cccnc5)cc4)nc(-c4cccc(-c5ccccc5)c4)n3)cc2-c2c1ccc1ccccc21]

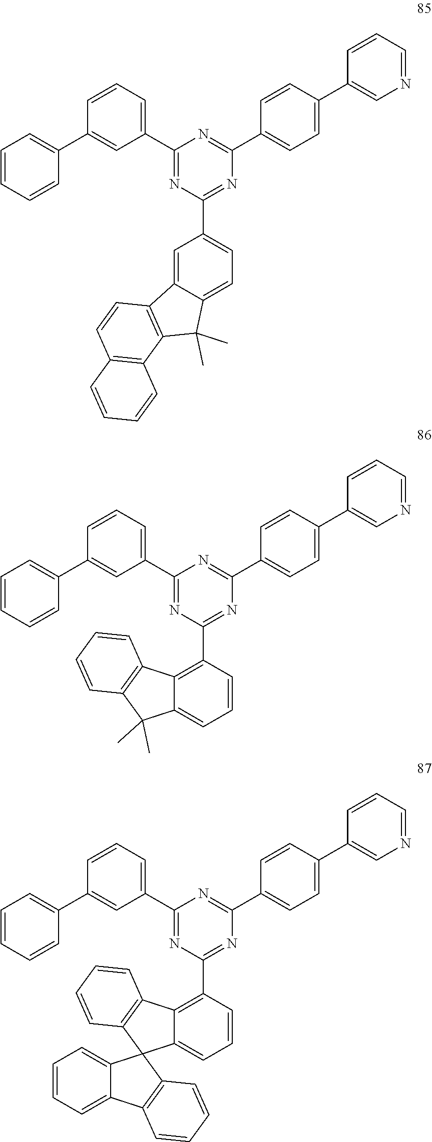 [SMILES: CC1(C)c2ccc(-c3nc(-c4ccc(-c5cccnc5)cc4)nc(-c4cccc(-c5ccccc5)c4)n3)cc2-c2ccc3ccccc3c21 ; CC1(C)c2ccccc2-c2c(-c3nc(-c4ccc(-c5cccnc5)cc4)nc(-c4cccc(-c5ccccc5)c4)n3)cccc21 ; c1ccc(-c2cccc(-c3nc(-c4ccc(-c5cccnc5)cc4)nc(-c4cccc5c4-c4ccccc4C54c5ccccc5-c5ccccc54)n3)c2)cc1]

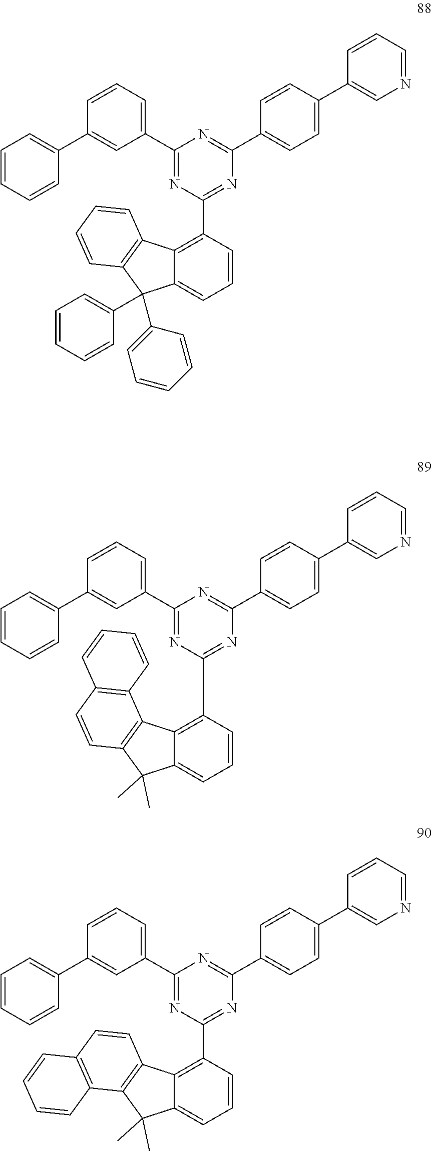 [SMILES: c1ccc(-c2cccc(-c3nc(-c4ccc(-c5cccnc5)cc4)nc(-c4cccc5c4-c4ccccc4C5(c4ccccc4)c4ccccc4)n3)c2)cc1 ; CC1(C)c2cccc(-c3nc(-c4ccc(-c5cccnc5)cc4)nc(-c4cccc(-c5ccccc5)c4)n3)c2-c2c1ccc1ccccc21 ; CC1(C)c2cccc(-c3nc(-c4ccc(-c5cccnc5)cc4)nc(-c4cccc(-c5ccccc5)c4)n3)c2-c2ccc3ccccc3c21]

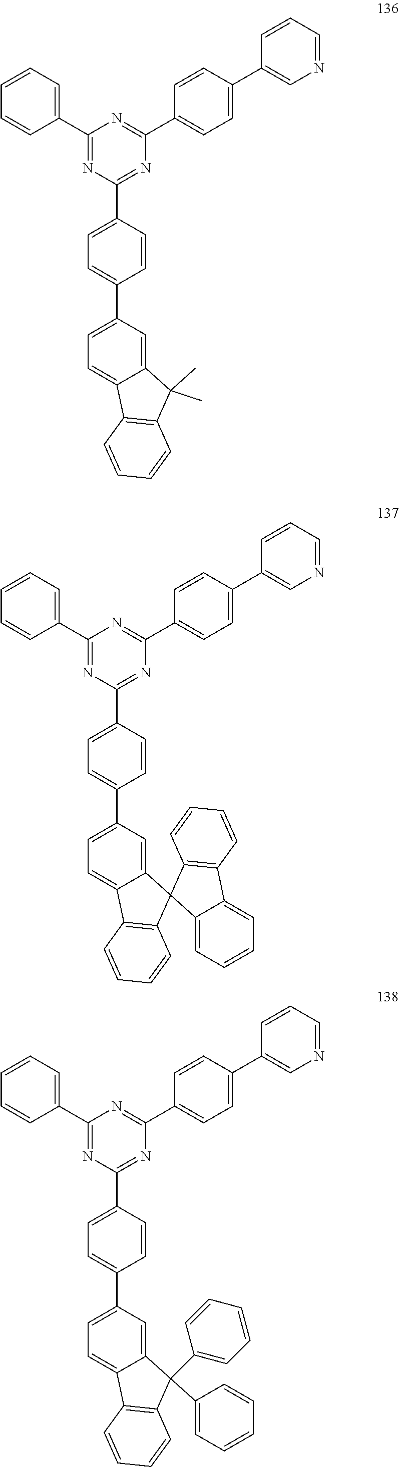 [SMILES: CC1(C)c2ccccc2-c2ccc(-c3ccc(-c4nc(-c5ccccc5)nc(-c5ccc(-c6cccnc6)cc5)n4)cc3)cc21 ; c1ccc(-c2nc(-c3ccc(-c4cccnc4)cc3)nc(-c3ccc(-c4ccc5c(c4)C4(c6ccccc6-c6ccccc64)c4ccccc4-5)cc3)n2)cc1 ; c1ccc(-c2nc(-c3ccc(-c4cccnc4)cc3)nc(-c3ccc(-c4ccc5c(c4)C(c4ccccc4)(c4ccccc4)c4ccccc4-5)cc3)n2)cc1]

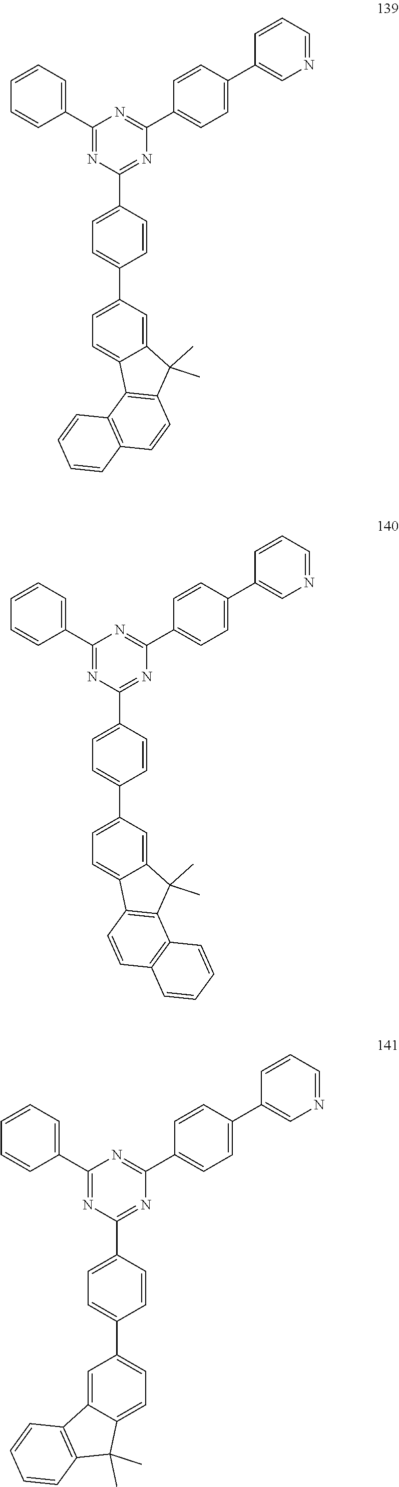 [SMILES: CC1(C)c2cc(-c3ccc(-c4nc(-c5ccccc5)nc(-c5ccc(-c6cccnc6)cc5)n4)cc3)ccc2-c2c1ccc1ccccc21 ; CC1(C)c2cc(-c3ccc(-c4nc(-c5ccccc5)nc(-c5ccc(-c6cccnc6)cc5)n4)cc3)ccc2-c2ccc3ccccc3c21 ; CC1(C)c2ccccc2-c2cc(-c3ccc(-c4nc(-c5ccccc5)nc(-c5ccc(-c6cccnc6)cc5)n4)cc3)ccc21]

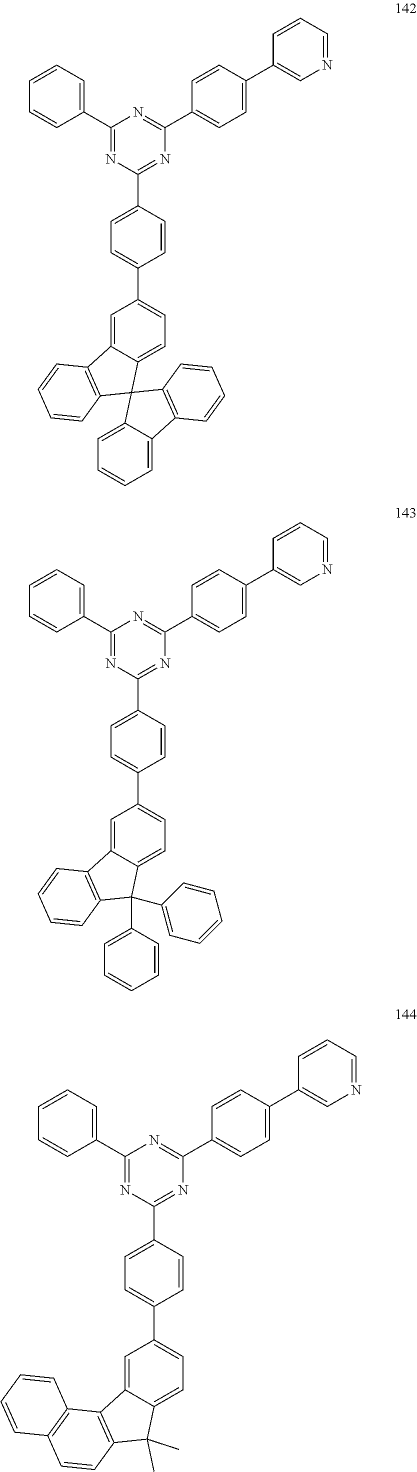 [SMILES: c1ccc(-c2nc(-c3ccc(-c4cccnc4)cc3)nc(-c3ccc(-c4ccc5c(c4)-c4ccccc4C54c5ccccc5-c5ccccc54)cc3)n2)cc1 ; c1ccc(-c2nc(-c3ccc(-c4cccnc4)cc3)nc(-c3ccc(-c4ccc5c(c4)-c4ccccc4C5(c4ccccc4)c4ccccc4)cc3)n2)cc1 ; CC1(C)c2ccc(-c3ccc(-c4nc(-c5ccccc5)nc(-c5ccc(-c6cccnc6)cc5)n4)cc3)cc2-c2c1ccc1ccccc21]

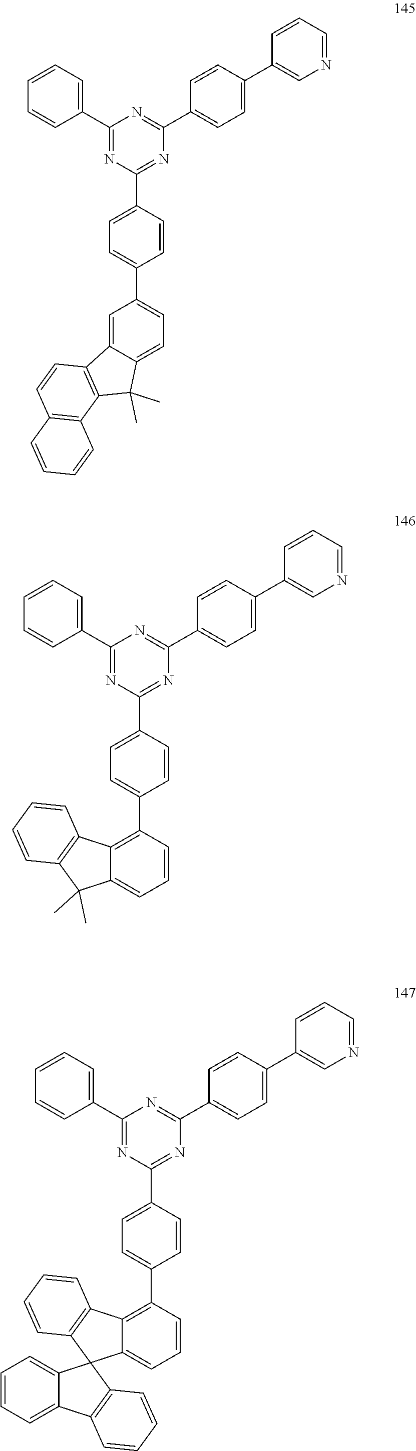 [SMILES: CC1(C)c2ccc(-c3ccc(-c4nc(-c5ccccc5)nc(-c5ccc(-c6cccnc6)cc5)n4)cc3)cc2-c2ccc3ccccc3c21 ; CC1(C)c2ccccc2-c2c(-c3ccc(-c4nc(-c5ccccc5)nc(-c5ccc(-c6cccnc6)cc5)n4)cc3)cccc21 ; c1ccc(-c2nc(-c3ccc(-c4cccnc4)cc3)nc(-c3ccc(-c4cccc5c4-c4ccccc4C54c5ccccc5-c5ccccc54)cc3)n2)cc1]

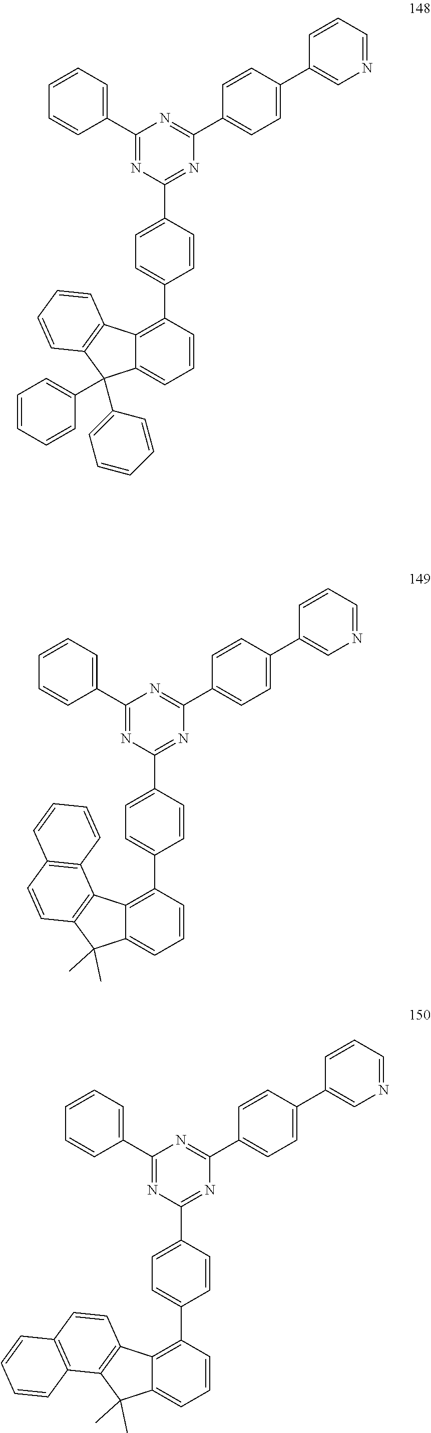 [SMILES: c1ccc(-c2nc(-c3ccc(-c4cccnc4)cc3)nc(-c3ccc(-c4cccc5c4-c4ccccc4C5(c4ccccc4)c4ccccc4)cc3)n2)cc1 ; CC1(C)c2cccc(-c3ccc(-c4nc(-c5ccccc5)nc(-c5ccc(-c6cccnc6)cc5)n4)cc3)c2-c2c1ccc1ccccc21 ; CC1(C)c2cccc(-c3ccc(-c4nc(-c5ccccc5)nc(-c5ccc(-c6cccnc6)cc5)n4)cc3)c2-c2ccc3ccccc3c21]

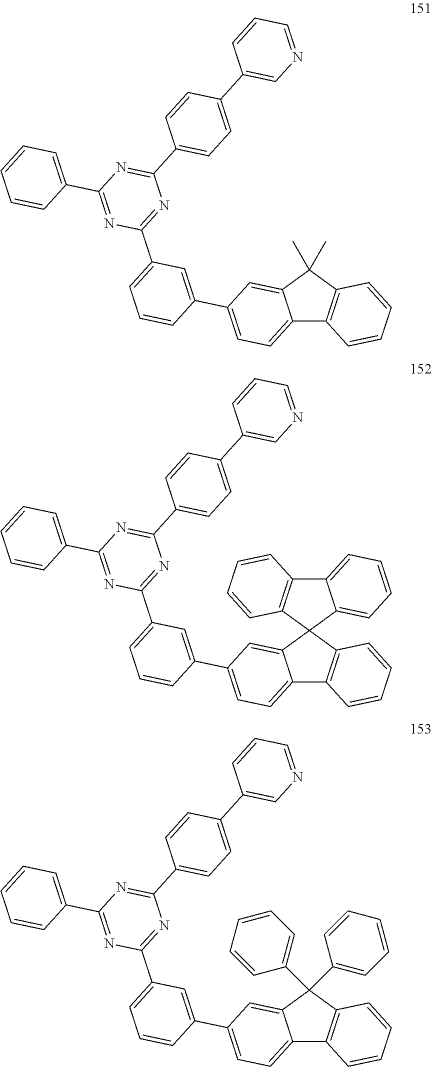

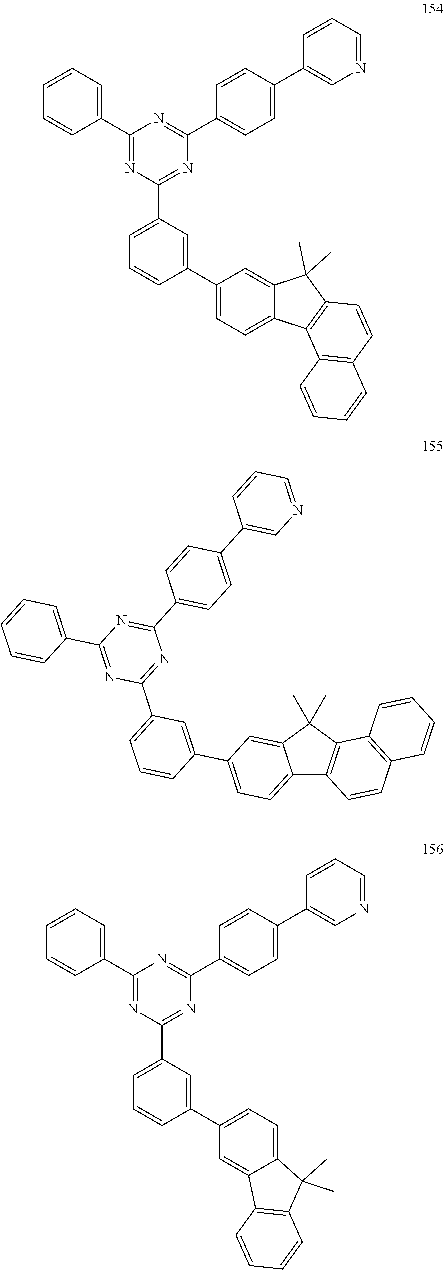 [SMILES: CC1(C)c2cc(-c3cccc(-c4nc(-c5ccccc5)nc(-c5ccc(-c6cccnc6)cc5)n4)c3)ccc2-c2c1ccc1ccccc21 ; CC1(C)c2cc(-c3cccc(-c4nc(-c5ccccc5)nc(-c5ccc(-c6cccnc6)cc5)n4)c3)ccc2-c2ccc3ccccc3c21 ; CC1(C)c2ccccc2-c2cc(-c3cccc(-c4nc(-c5ccccc5)nc(-c5ccc(-c6cccnc6)cc5)n4)c3)ccc21]

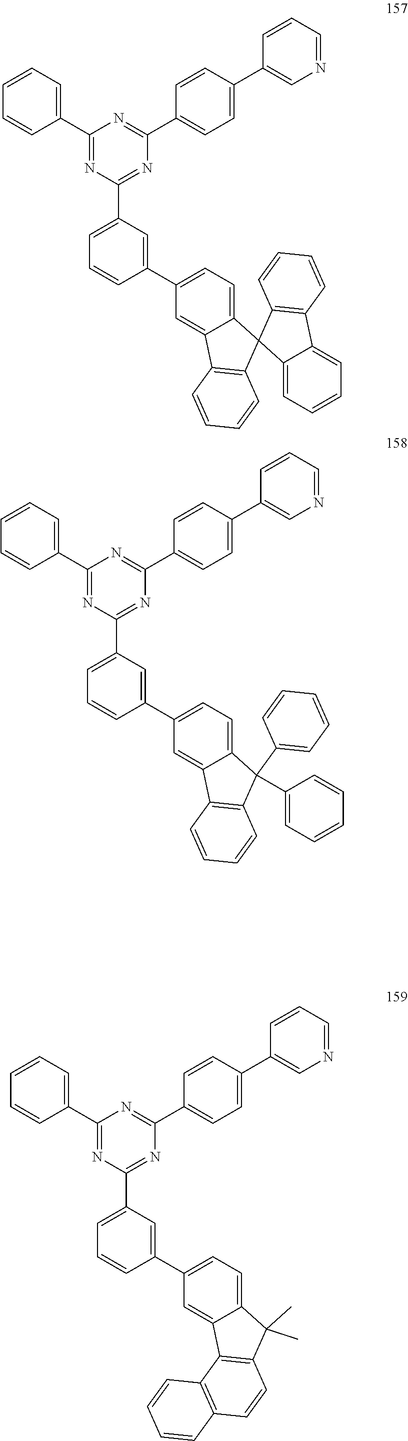

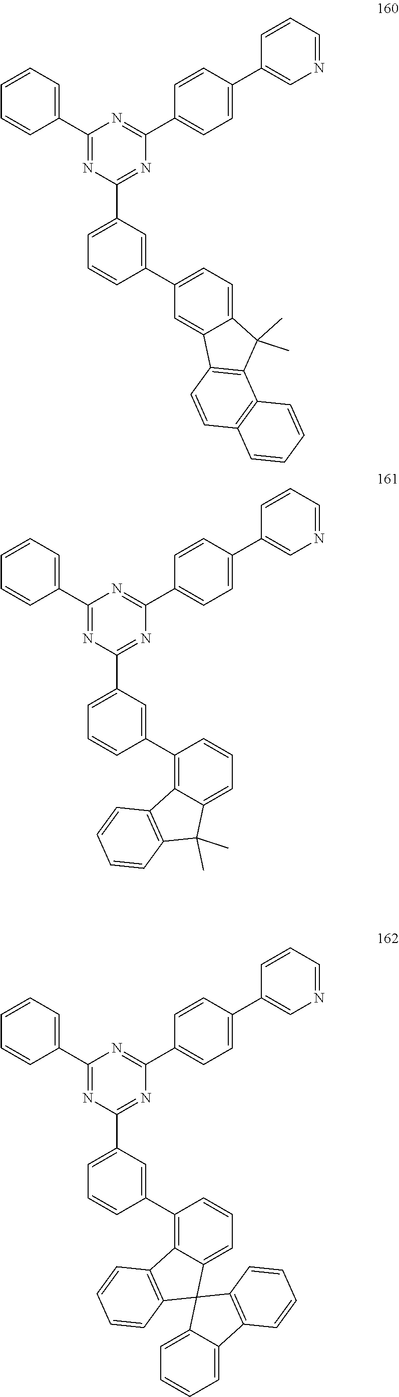

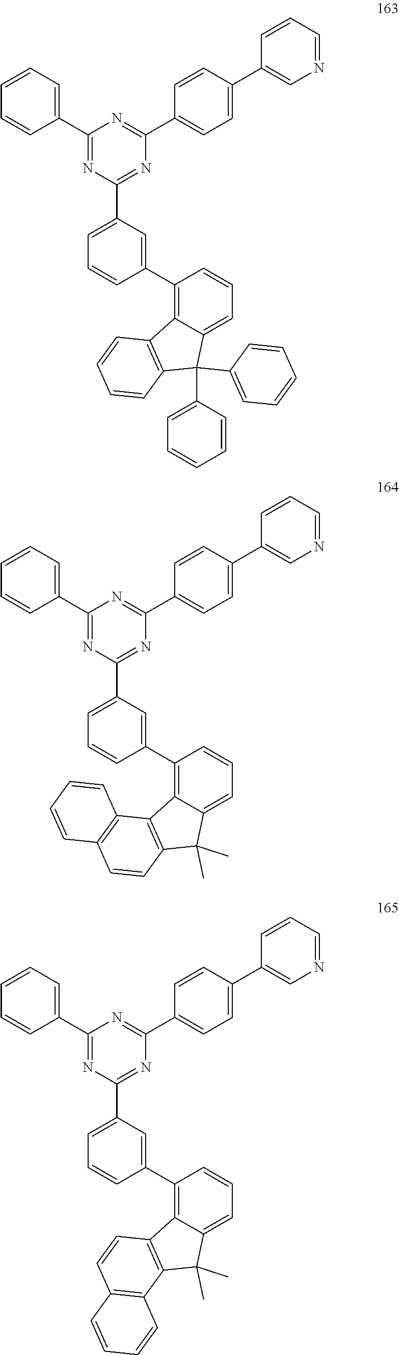 [SMILES: c1ccc(-c2nc(-c3ccc(-c4cccnc4)cc3)nc(-c3cccc(-c4cccc5c4-c4ccccc4C5(c4ccccc4)c4ccccc4)c3)n2)cc1 ; CC1(C)c2cccc(-c3cccc(-c4nc(-c5ccccc5)nc(-c5ccc(-c6cccnc6)cc5)n4)c3)c2-c2c1ccc1ccccc21 ; CC1(C)c2cccc(-c3cccc(-c4nc(-c5ccccc5)nc(-c5ccc(-c6cccnc6)cc5)n4)c3)c2-c2ccc3ccccc3c21]

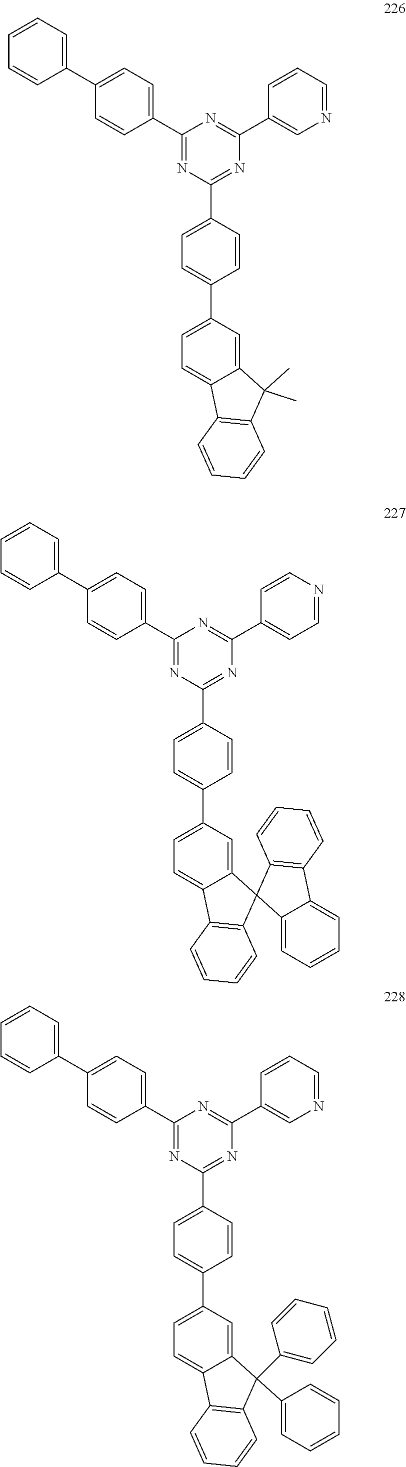 [SMILES: CC1(C)c2ccccc2-c2ccc(-c3ccc(-c4nc(-c5ccc(-c6ccccc6)cc5)nc(-c5cccnc5)n4)cc3)cc21 ; c1ccc(-c2ccc(-c3nc(-c4ccncc4)nc(-c4ccc(-c5ccc6c(c5)C5(c7ccccc7-c7ccccc75)c5ccccc5-6)cc4)n3)cc2)cc1 ; c1ccc(-c2ccc(-c3nc(-c4ccc(-c5ccc6c(c5)C(c5ccccc5)(c5ccccc5)c5ccccc5-6)cc4)nc(-c4cccnc4)n3)cc2)cc1]

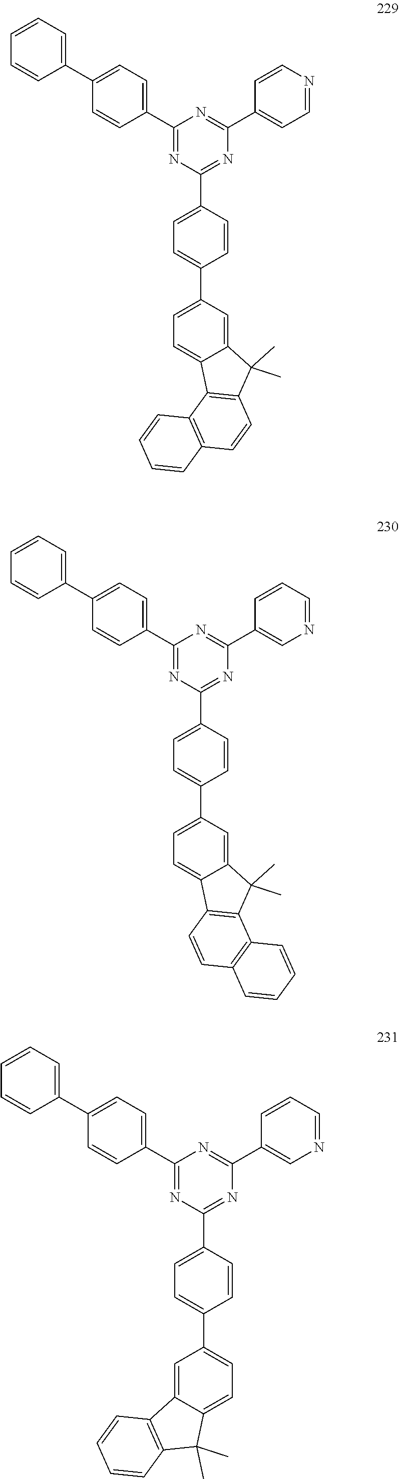 [SMILES: CC1(C)c2cc(-c3ccc(-c4nc(-c5ccncc5)nc(-c5ccc(-c6ccccc6)cc5)n4)cc3)ccc2-c2c1ccc1ccccc21 ; CC1(C)c2cc(-c3ccc(-c4nc(-c5ccc(-c6ccccc6)cc5)nc(-c5cccnc5)n4)cc3)ccc2-c2ccc3ccccc3c21 ; CC1(C)c2ccccc2-c2cc(-c3ccc(-c4nc(-c5ccc(-c6ccccc6)cc5)nc(-c5cccnc5)n4)cc3)ccc21]

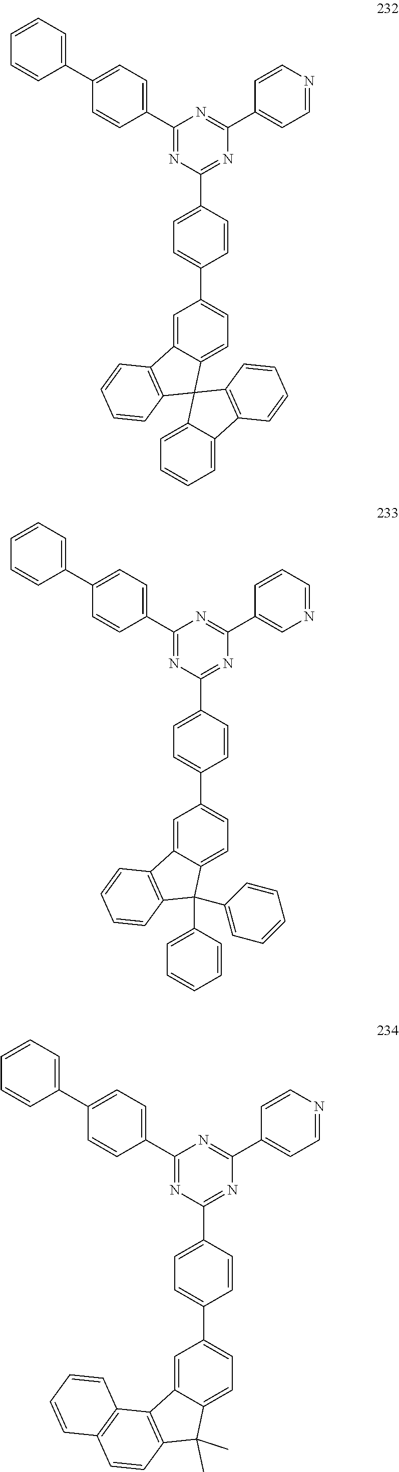 [SMILES: c1ccc(-c2ccc(-c3nc(-c4ccncc4)nc(-c4ccc(-c5ccc6c(c5)-c5ccccc5C65c6ccccc6-c6ccccc65)cc4)n3)cc2)cc1 ; c1ccc(-c2ccc(-c3nc(-c4ccc(-c5ccc6c(c5)-c5ccccc5C6(c5ccccc5)c5ccccc5)cc4)nc(-c4cccnc4)n3)cc2)cc1 ; CC1(C)c2ccc(-c3ccc(-c4nc(-c5ccncc5)nc(-c5ccc(-c6ccccc6)cc5)n4)cc3)cc2-c2c1ccc1ccccc21]

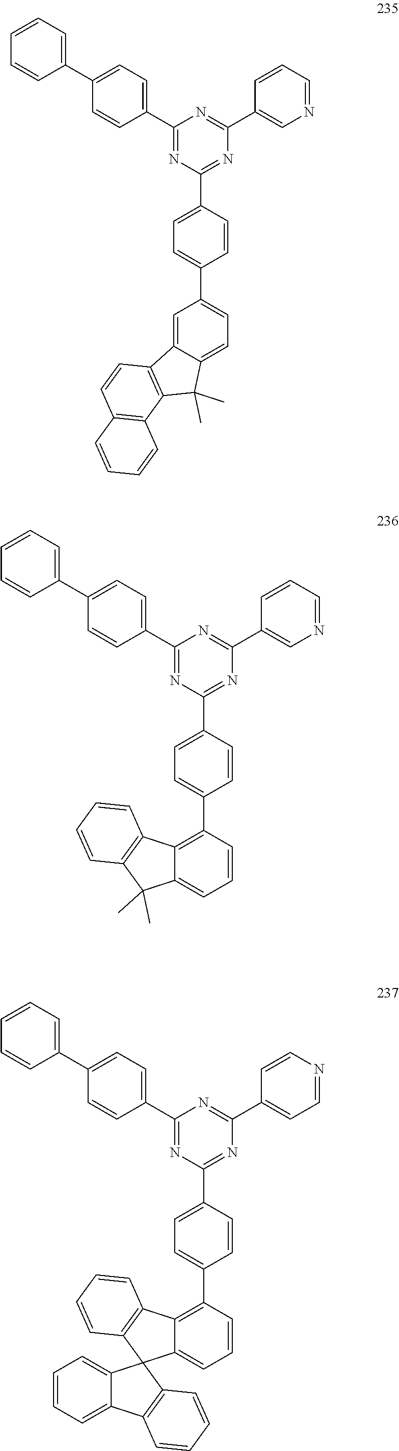 [SMILES: CC1(C)c2ccc(-c3ccc(-c4nc(-c5ccc(-c6ccccc6)cc5)nc(-c5cccnc5)n4)cc3)cc2-c2ccc3ccccc3c21 ; CC1(C)c2ccccc2-c2c(-c3ccc(-c4nc(-c5ccc(-c6ccccc6)cc5)nc(-c5cccnc5)n4)cc3)cccc21 ; c1ccc(-c2ccc(-c3nc(-c4ccncc4)nc(-c4ccc(-c5cccc6c5-c5ccccc5C65c6ccccc6-c6ccccc65)cc4)n3)cc2)cc1]

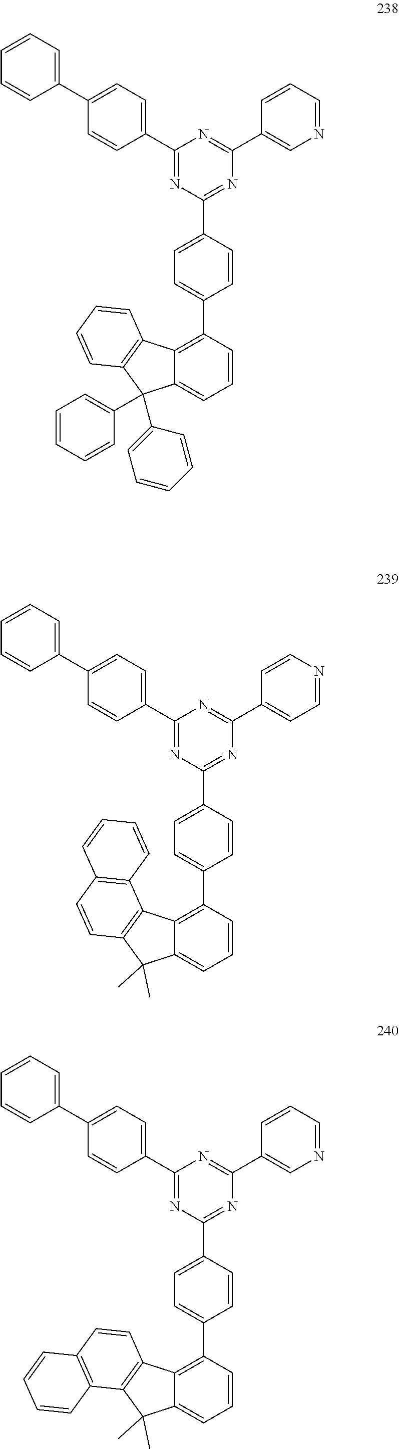 [SMILES: c1ccc(-c2ccc(-c3nc(-c4ccc(-c5cccc6c5-c5ccccc5C6(c5ccccc5)c5ccccc5)cc4)nc(-c4cccnc4)n3)cc2)cc1 ; CC1(C)c2cccc(-c3ccc(-c4nc(-c5ccncc5)nc(-c5ccc(-c6ccccc6)cc5)n4)cc3)c2-c2c1ccc1ccccc21 ; CC1(C)c2cccc(-c3ccc(-c4nc(-c5ccc(-c6ccccc6)cc5)nc(-c5cccnc5)n4)cc3)c2-c2ccc3ccccc3c21]

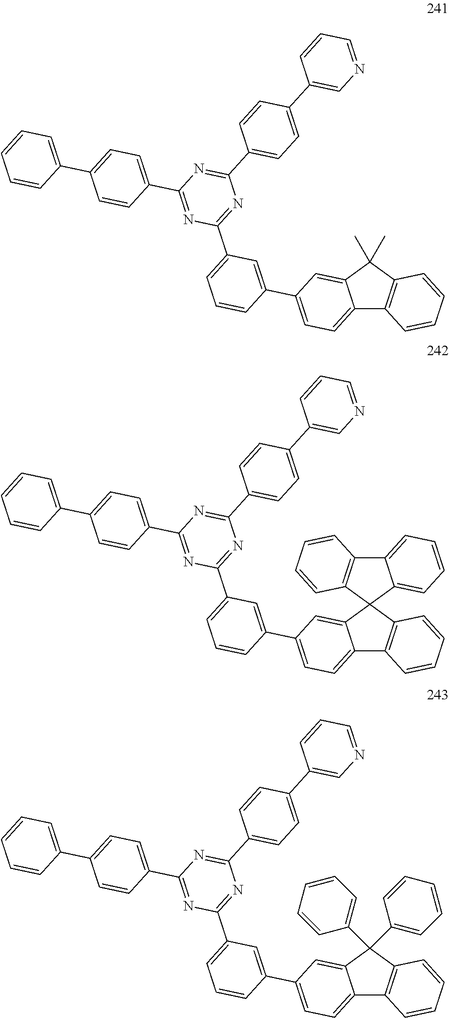 [SMILES: CC1(C)c2ccccc2-c2ccc(-c3cccc(-c4nc(-c5ccc(-c6ccccc6)cc5)nc(-c5ccc(-c6cccnc6)cc5)n4)c3)cc21 ; c1ccc(-c2ccc(-c3nc(-c4ccc(-c5cccnc5)cc4)nc(-c4cccc(-c5ccc6c(c5)C5(c7ccccc7-c7ccccc75)c5ccccc5-6)c4)n3)cc2)cc1 ; c1ccc(-c2ccc(-c3nc(-c4ccc(-c5cccnc5)cc4)nc(-c4cccc(-c5ccc6c(c5)C(c5ccccc5)(c5ccccc5)c5ccccc5-6)c4)n3)cc2)cc1]

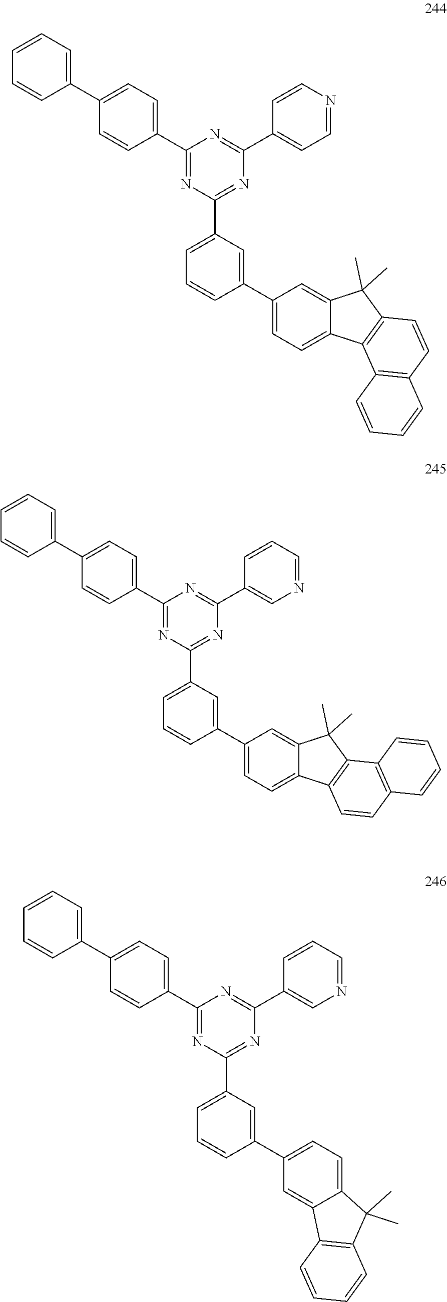 [SMILES: CC1(C)c2cc(-c3cccc(-c4nc(-c5ccncc5)nc(-c5ccc(-c6ccccc6)cc5)n4)c3)ccc2-c2c1ccc1ccccc21 ; CC1(C)c2cc(-c3cccc(-c4nc(-c5ccc(-c6ccccc6)cc5)nc(-c5cccnc5)n4)c3)ccc2-c2ccc3ccccc3c21 ; CC1(C)c2ccccc2-c2cc(-c3cccc(-c4nc(-c5ccc(-c6ccccc6)cc5)nc(-c5cccnc5)n4)c3)ccc21]

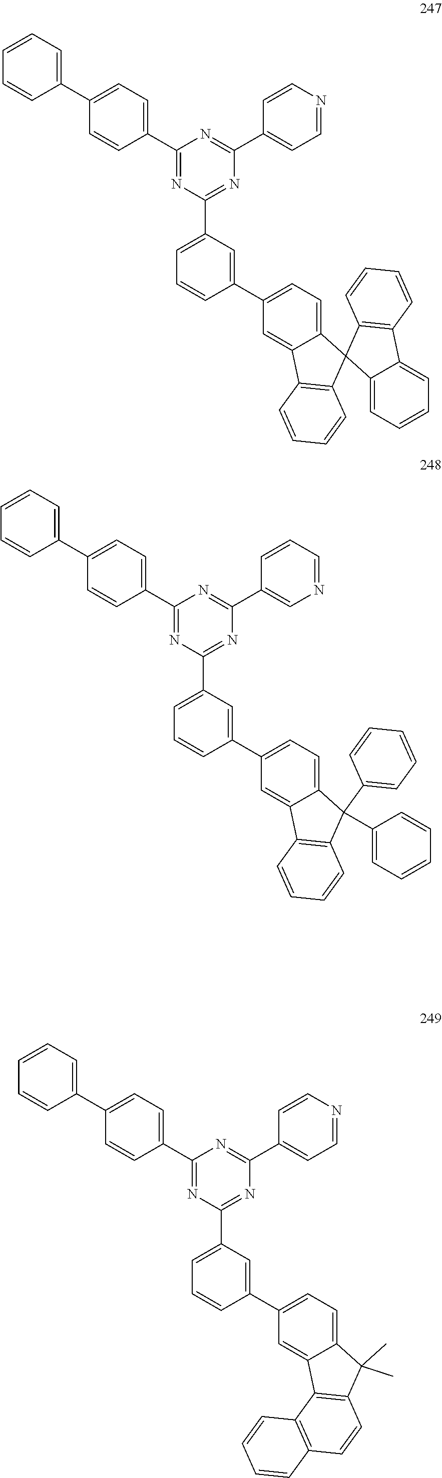 [SMILES: c1ccc(-c2ccc(-c3nc(-c4ccncc4)nc(-c4cccc(-c5ccc6c(c5)-c5ccccc5C65c6ccccc6-c6ccccc65)c4)n3)cc2)cc1 ; c1ccc(-c2ccc(-c3nc(-c4cccnc4)nc(-c4cccc(-c5ccc6c(c5)-c5ccccc5C6(c5ccccc5)c5ccccc5)c4)n3)cc2)cc1 ; CC1(C)c2ccc(-c3cccc(-c4nc(-c5ccncc5)nc(-c5ccc(-c6ccccc6)cc5)n4)c3)cc2-c2c1ccc1ccccc21]

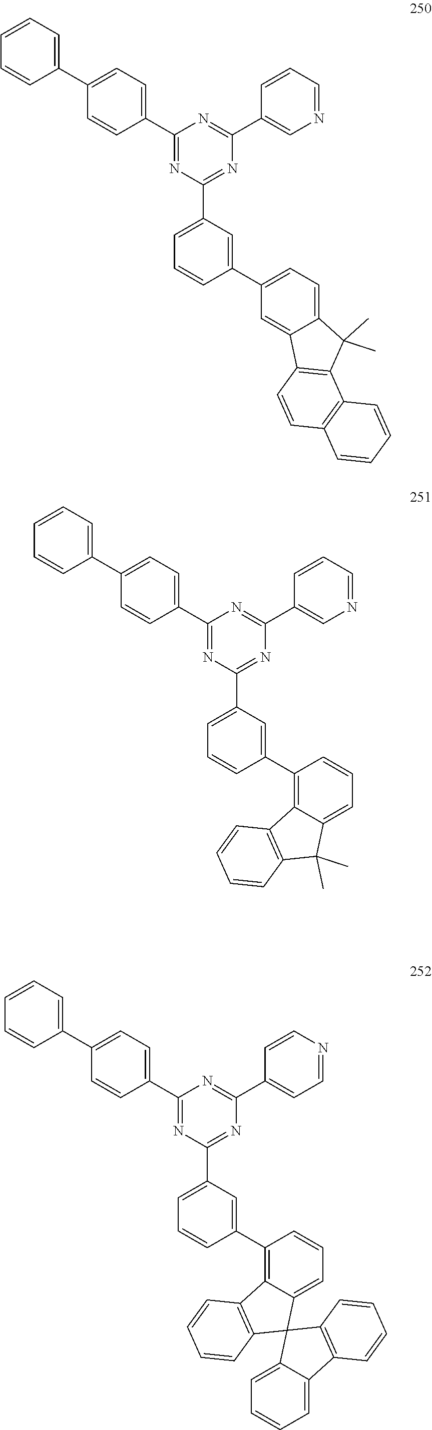 [SMILES: CC1(C)c2ccc(-c3cccc(-c4nc(-c5ccc(-c6ccccc6)cc5)nc(-c5cccnc5)n4)c3)cc2-c2ccc3ccccc3c21 ; CC1(C)c2ccccc2-c2c(-c3cccc(-c4nc(-c5ccc(-c6ccccc6)cc5)nc(-c5cccnc5)n4)c3)cccc21 ; c1ccc(-c2ccc(-c3nc(-c4ccncc4)nc(-c4cccc(-c5cccc6c5-c5ccccc5C65c6ccccc6-c6ccccc65)c4)n3)cc2)cc1]

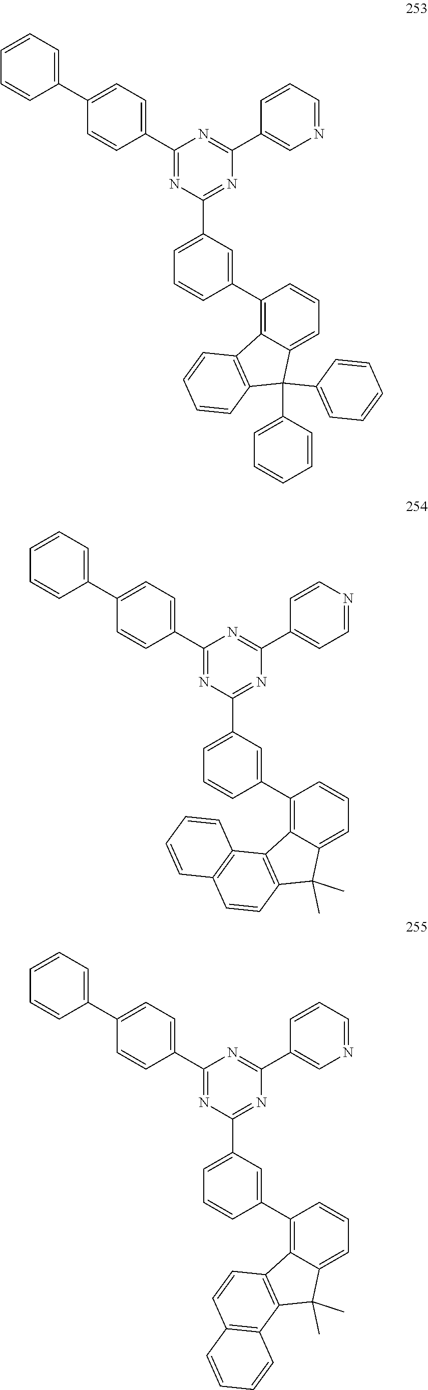 [SMILES: c1ccc(-c2ccc(-c3nc(-c4cccnc4)nc(-c4cccc(-c5cccc6c5-c5ccccc5C6(c5ccccc5)c5ccccc5)c4)n3)cc2)cc1 ; CC1(C)c2cccc(-c3cccc(-c4nc(-c5ccncc5)nc(-c5ccc(-c6ccccc6)cc5)n4)c3)c2-c2c1ccc1ccccc21 ; CC1(C)c2cccc(-c3cccc(-c4nc(-c5ccc(-c6ccccc6)cc5)nc(-c5cccnc5)n4)c3)c2-c2ccc3ccccc3c21]

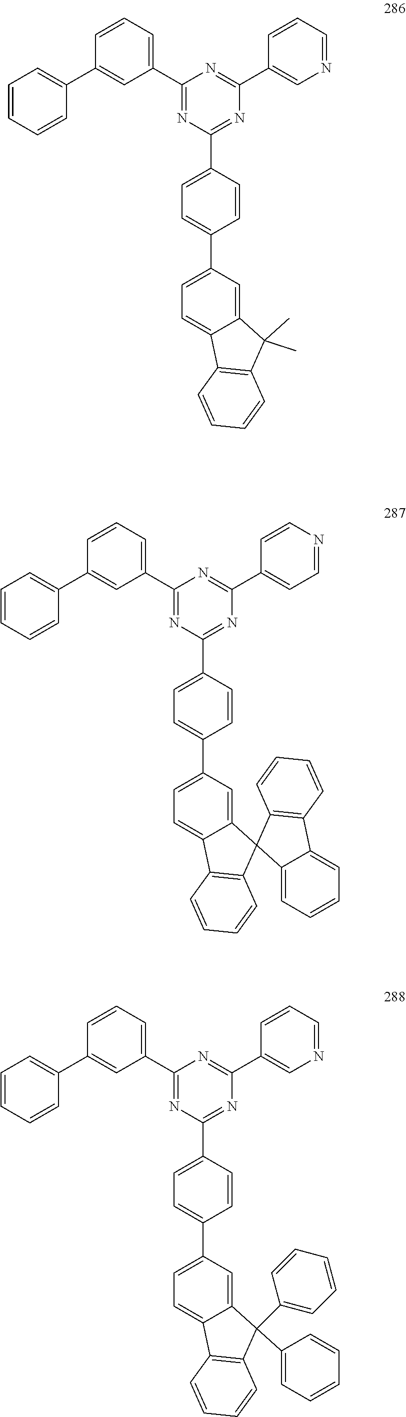 [SMILES: CC1(C)c2ccccc2-c2ccc(-c3ccc(-c4nc(-c5cccnc5)nc(-c5cccc(-c6ccccc6)c5)n4)cc3)cc21 ; c1ccc(-c2cccc(-c3nc(-c4ccncc4)nc(-c4ccc(-c5ccc6c(c5)C5(c7ccccc7-c7ccccc75)c5ccccc5-6)cc4)n3)c2)cc1 ; c1ccc(-c2cccc(-c3nc(-c4ccc(-c5ccc6c(c5)C(c5ccccc5)(c5ccccc5)c5ccccc5-6)cc4)nc(-c4cccnc4)n3)c2)cc1]

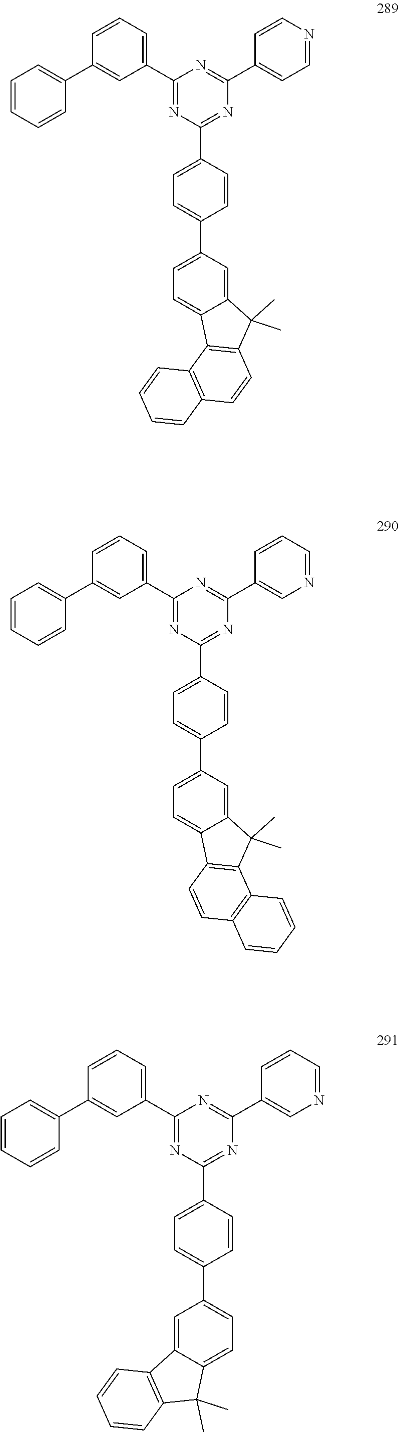 [SMILES: CC1(C)c2cc(-c3ccc(-c4nc(-c5ccncc5)nc(-c5cccc(-c6ccccc6)c5)n4)cc3)ccc2-c2c1ccc1ccccc21 ; CC1(C)c2cc(-c3ccc(-c4nc(-c5cccnc5)nc(-c5cccc(-c6ccccc6)c5)n4)cc3)ccc2-c2ccc3ccccc3c21 ; CC1(C)c2ccccc2-c2cc(-c3ccc(-c4nc(-c5cccnc5)nc(-c5cccc(-c6ccccc6)c5)n4)cc3)ccc21]

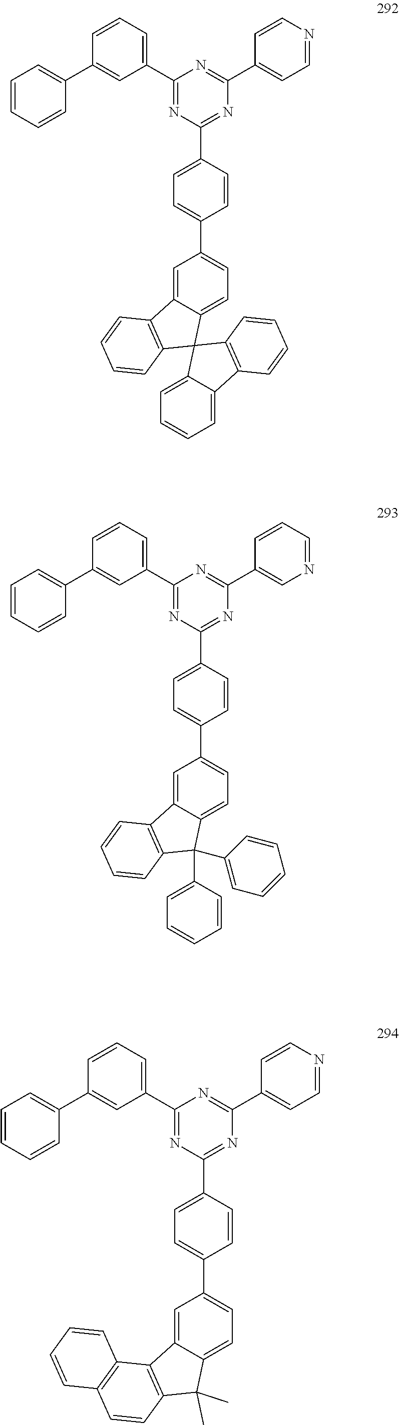 [SMILES: c1ccc(-c2cccc(-c3nc(-c4ccncc4)nc(-c4ccc(-c5ccc6c(c5)-c5ccccc5C65c6ccccc6-c6ccccc65)cc4)n3)c2)cc1 ; c1ccc(-c2cccc(-c3nc(-c4ccc(-c5ccc6c(c5)-c5ccccc5C6(c5ccccc5)c5ccccc5)cc4)nc(-c4cccnc4)n3)c2)cc1 ; CC1(C)c2ccc(-c3ccc(-c4nc(-c5ccncc5)nc(-c5cccc(-c6ccccc6)c5)n4)cc3)cc2-c2c1ccc1ccccc21]

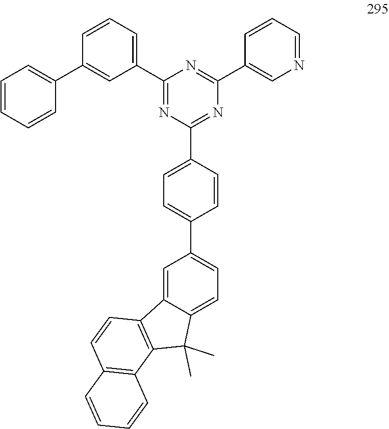 [SMILES: CC1(C)c2ccc(-c3ccc(-c4nc(-c5cccnc5)nc(-c5cccc(-c6ccccc6)c5)n4)cc3)cc2-c2ccc3ccccc3c21]

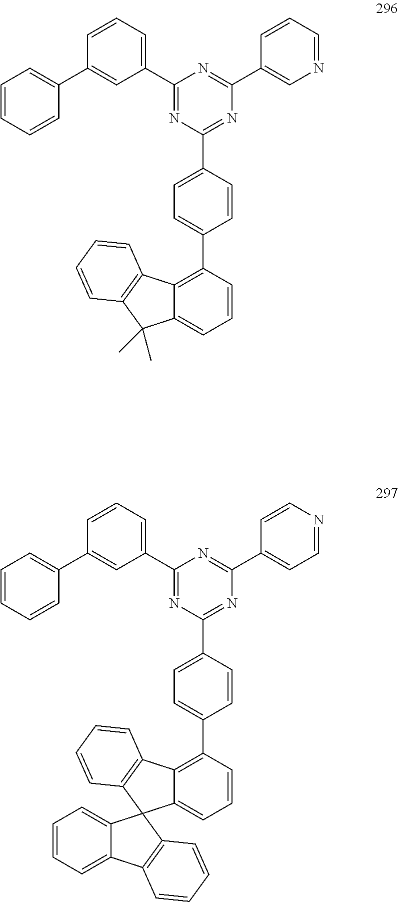 [SMILES: CC1(C)c2ccccc2-c2c(-c3ccc(-c4nc(-c5cccnc5)nc(-c5cccc(-c6ccccc6)c5)n4)cc3)cccc21 ; c1ccc(-c2cccc(-c3nc(-c4ccncc4)nc(-c4ccc(-c5cccc6c5-c5ccccc5C65c6ccccc6-c6ccccc65)cc4)n3)c2)cc1]

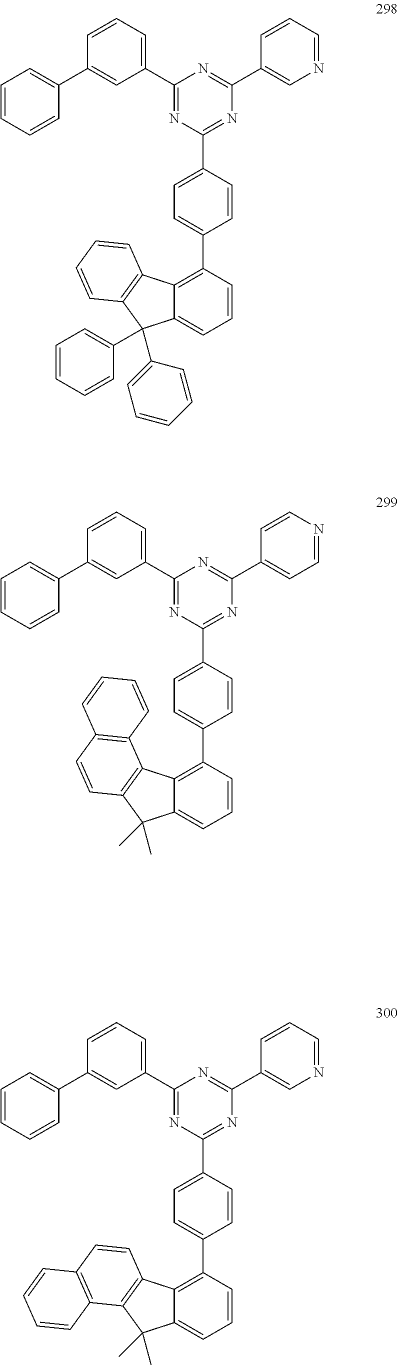 [SMILES: c1ccc(-c2cccc(-c3nc(-c4ccc(-c5cccc6c5-c5ccccc5C6(c5ccccc5)c5ccccc5)cc4)nc(-c4cccnc4)n3)c2)cc1 ; CC1(C)c2cccc(-c3ccc(-c4nc(-c5ccncc5)nc(-c5cccc(-c6ccccc6)c5)n4)cc3)c2-c2c1ccc1ccccc21 ; CC1(C)c2cccc(-c3ccc(-c4nc(-c5cccnc5)nc(-c5cccc(-c6ccccc6)c5)n4)cc3)c2-c2ccc3ccccc3c21]

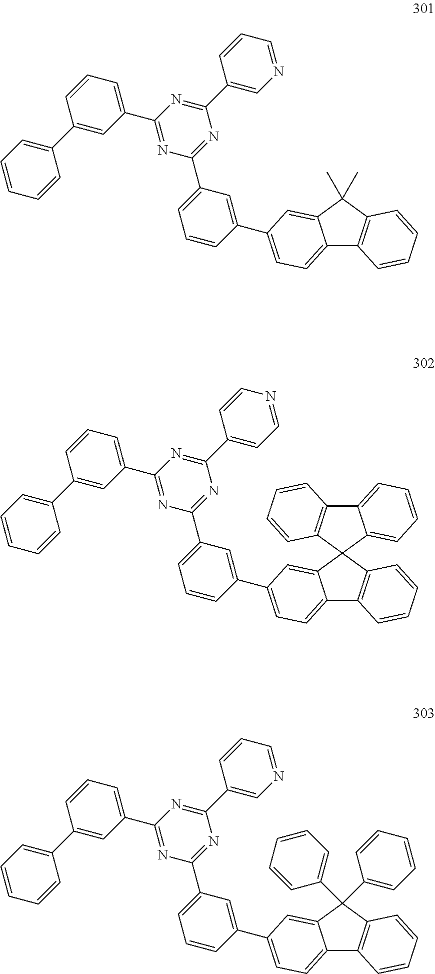 [SMILES: CC1(C)c2ccccc2-c2ccc(-c3cccc(-c4nc(-c5cccnc5)nc(-c5cccc(-c6ccccc6)c5)n4)c3)cc21 ; c1ccc(-c2cccc(-c3nc(-c4ccncc4)nc(-c4cccc(-c5ccc6c(c5)C5(c7ccccc7-c7ccccc75)c5ccccc5-6)c4)n3)c2)cc1 ; c1ccc(-c2cccc(-c3nc(-c4cccnc4)nc(-c4cccc(-c5ccc6c(c5)C(c5ccccc5)(c5ccccc5)c5ccccc5-6)c4)n3)c2)cc1]

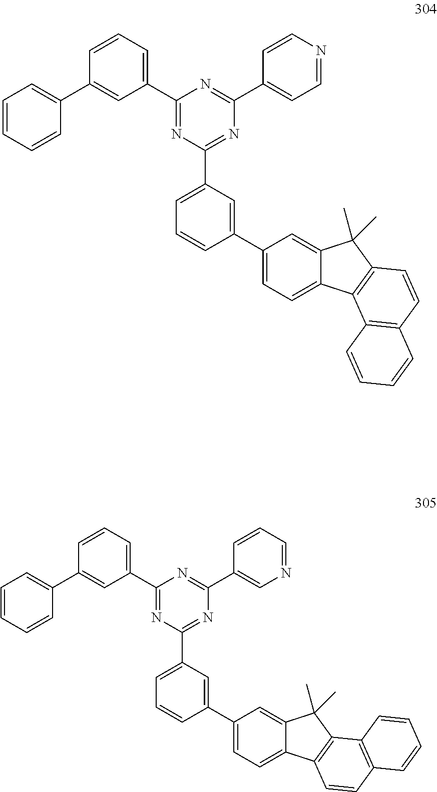 [SMILES: CC1(C)c2cc(-c3cccc(-c4nc(-c5ccncc5)nc(-c5cccc(-c6ccccc6)c5)n4)c3)ccc2-c2c1ccc1ccccc21 ; CC1(C)c2cc(-c3cccc(-c4nc(-c5cccnc5)nc(-c5cccc(-c6ccccc6)c5)n4)c3)ccc2-c2ccc3ccccc3c21]

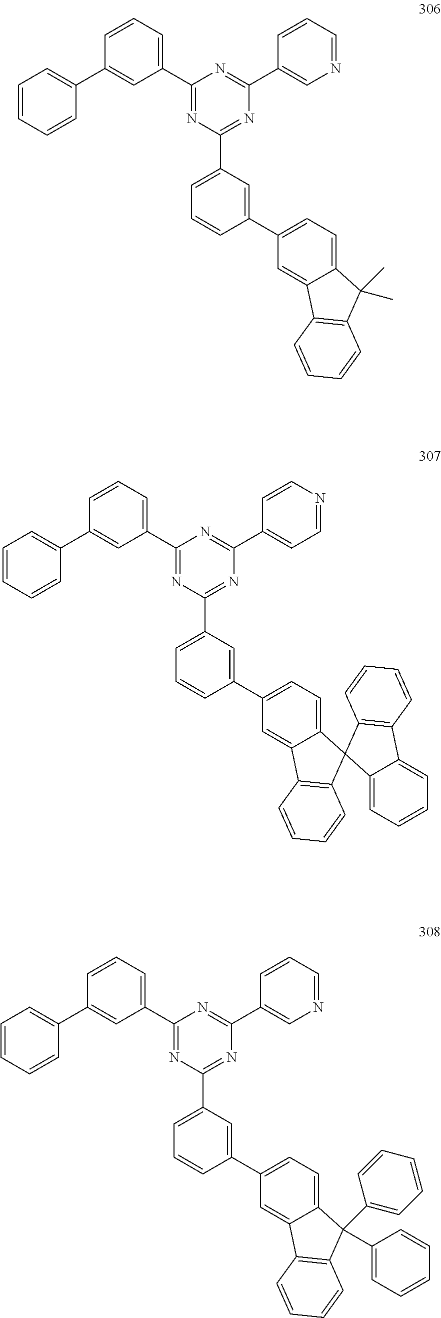 [SMILES: CC1(C)c2ccccc2-c2cc(-c3cccc(-c4nc(-c5cccnc5)nc(-c5cccc(-c6ccccc6)c5)n4)c3)ccc21 ; c1ccc(-c2cccc(-c3nc(-c4ccncc4)nc(-c4cccc(-c5ccc6c(c5)-c5ccccc5C65c6ccccc6-c6ccccc65)c4)n3)c2)cc1 ; c1ccc(-c2cccc(-c3nc(-c4cccnc4)nc(-c4cccc(-c5ccc6c(c5)-c5ccccc5C6(c5ccccc5)c5ccccc5)c4)n3)c2)cc1]

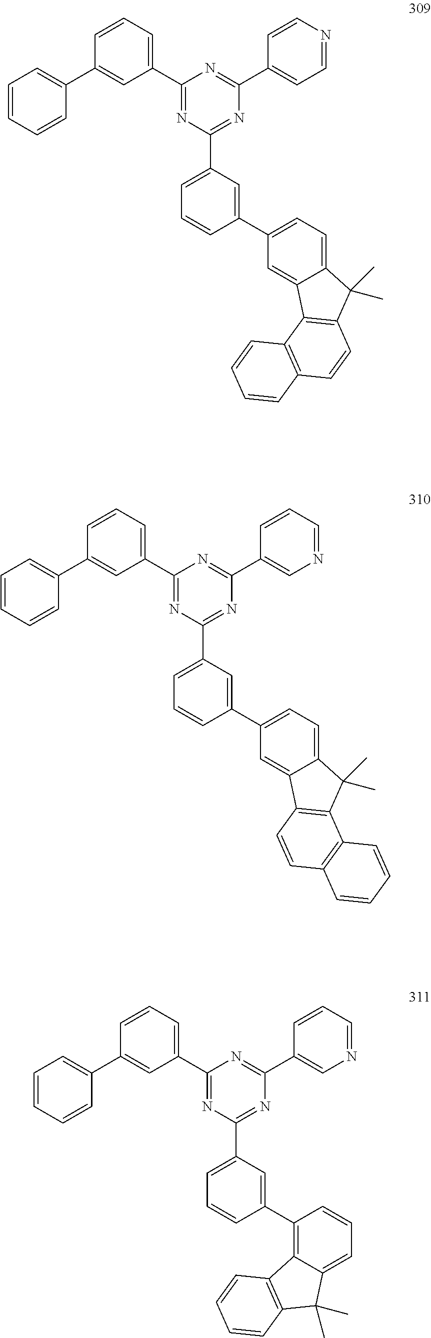 [SMILES: CC1(C)c2ccc(-c3cccc(-c4nc(-c5ccncc5)nc(-c5cccc(-c6ccccc6)c5)n4)c3)cc2-c2c1ccc1ccccc21 ; CC1(C)c2ccc(-c3cccc(-c4nc(-c5cccnc5)nc(-c5cccc(-c6ccccc6)c5)n4)c3)cc2-c2ccc3ccccc3c21 ; CC1(C)c2ccccc2-c2c(-c3cccc(-c4nc(-c5cccnc5)nc(-c5cccc(-c6ccccc6)c5)n4)c3)cccc21]

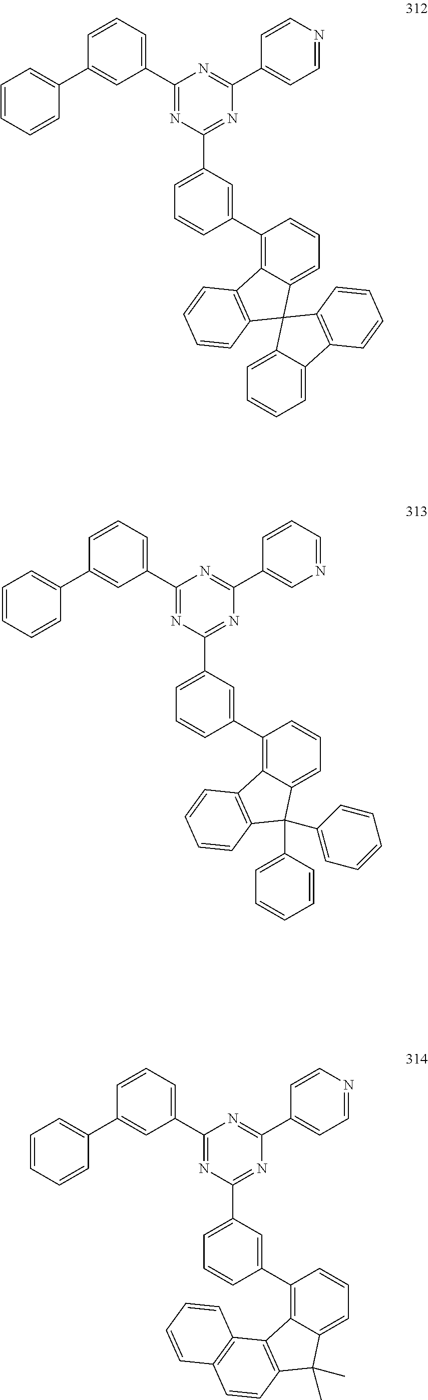 [SMILES: c1ccc(-c2cccc(-c3nc(-c4ccncc4)nc(-c4cccc(-c5cccc6c5-c5ccccc5C65c6ccccc6-c6ccccc65)c4)n3)c2)cc1 ; c1ccc(-c2cccc(-c3nc(-c4cccnc4)nc(-c4cccc(-c5cccc6c5-c5ccccc5C6(c5ccccc5)c5ccccc5)c4)n3)c2)cc1 ; CC1(C)c2cccc(-c3cccc(-c4nc(-c5ccncc5)nc(-c5cccc(-c6ccccc6)c5)n4)c3)c2-c2c1ccc1ccccc21]

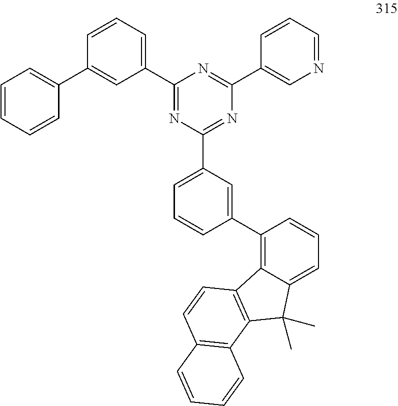 [SMILES: CC1(C)c2cccc(-c3cccc(-c4nc(-c5cccnc5)nc(-c5cccc(-c6ccccc6)c5)n4)c3)c2-c2ccc3ccccc3c21]

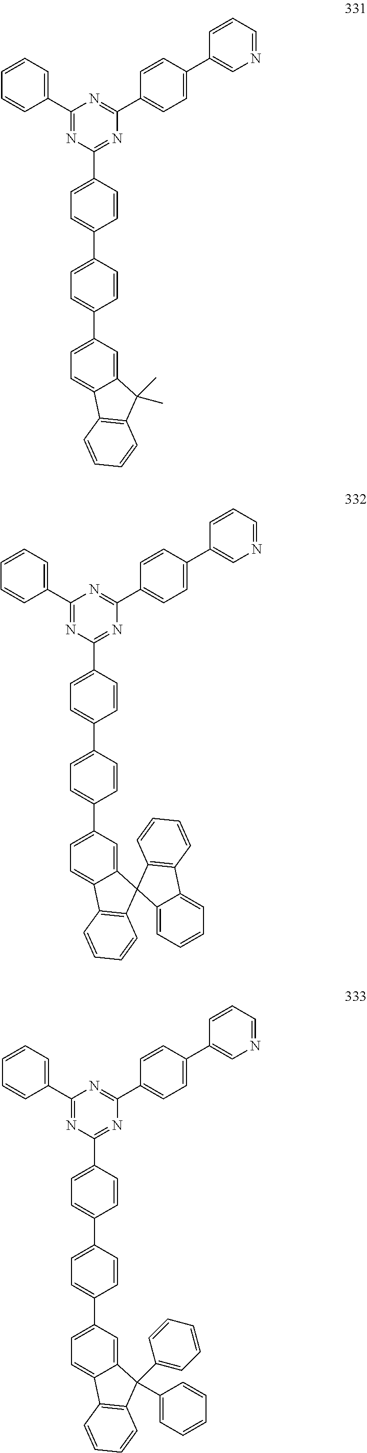 [SMILES: CC1(C)c2ccccc2-c2ccc(-c3ccc(-c4ccc(-c5nc(-c6ccccc6)nc(-c6ccc(-c7cccnc7)cc6)n5)cc4)cc3)cc21 ; c1ccc(-c2nc(-c3ccc(-c4ccc(-c5ccc6c(c5)C5(c7ccccc7-c7ccccc75)c5ccccc5-6)cc4)cc3)nc(-c3ccc(-c4cccnc4)cc3)n2)cc1 ; c1ccc(-c2nc(-c3ccc(-c4ccc(-c5ccc6c(c5)C(c5ccccc5)(c5ccccc5)c5ccccc5-6)cc4)cc3)nc(-c3ccc(-c4cccnc4)cc3)n2)cc1]

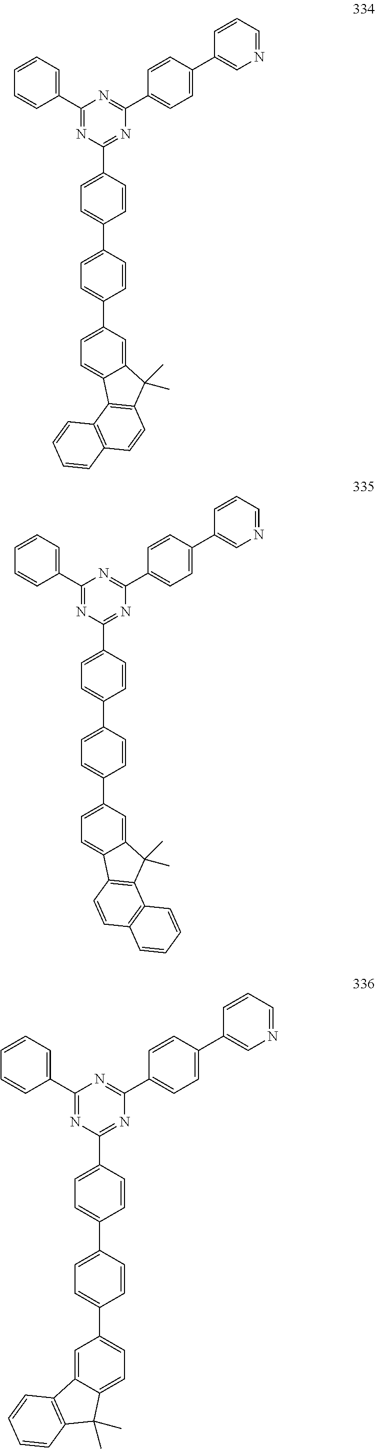 [SMILES: CC1(C)c2cc(-c3ccc(-c4ccc(-c5nc(-c6ccccc6)nc(-c6ccc(-c7cccnc7)cc6)n5)cc4)cc3)ccc2-c2c1ccc1ccccc21 ; CC1(C)c2cc(-c3ccc(-c4ccc(-c5nc(-c6ccccc6)nc(-c6ccc(-c7cccnc7)cc6)n5)cc4)cc3)ccc2-c2ccc3ccccc3c21 ; CC1(C)c2ccccc2-c2cc(-c3ccc(-c4ccc(-c5nc(-c6ccccc6)nc(-c6ccc(-c7cccnc7)cc6)n5)cc4)cc3)ccc21]

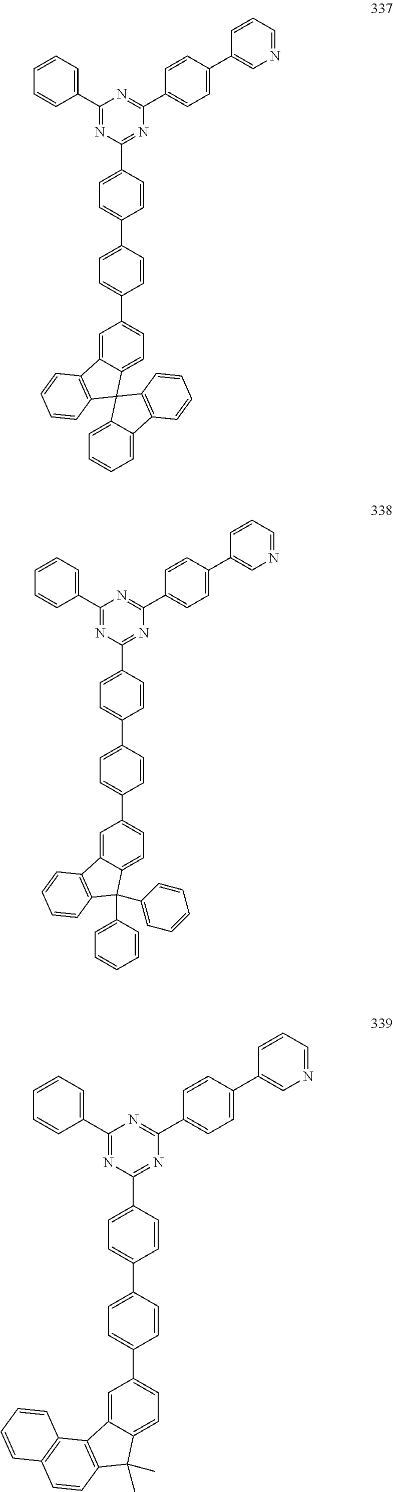 [SMILES: c1ccc(-c2nc(-c3ccc(-c4ccc(-c5ccc6c(c5)-c5ccccc5C65c6ccccc6-c6ccccc65)cc4)cc3)nc(-c3ccc(-c4cccnc4)cc3)n2)cc1 ; c1ccc(-c2nc(-c3ccc(-c4ccc(-c5ccc6c(c5)-c5ccccc5C6(c5ccccc5)c5ccccc5)cc4)cc3)nc(-c3ccc(-c4cccnc4)cc3)n2)cc1 ; CC1(C)c2ccc(-c3ccc(-c4ccc(-c5nc(-c6ccccc6)nc(-c6ccc(-c7cccnc7)cc6)n5)cc4)cc3)cc2-c2c1ccc1ccccc21]

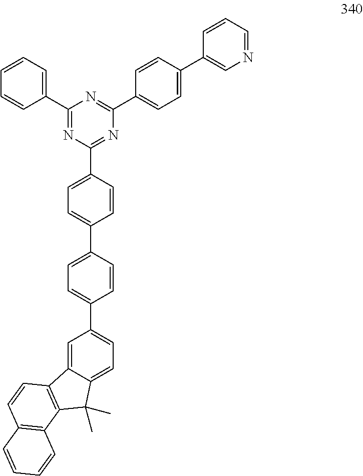 [SMILES: CC1(C)c2ccc(-c3ccc(-c4ccc(-c5nc(-c6ccccc6)nc(-c6ccc(-c7cccnc7)cc6)n5)cc4)cc3)cc2-c2ccc3ccccc3c21]

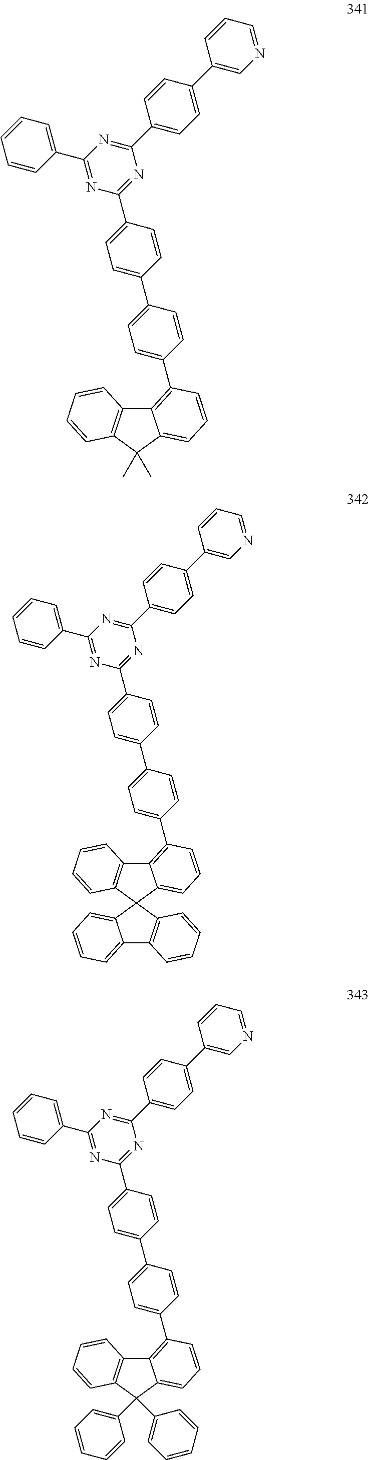 [SMILES: CC1(C)c2ccccc2-c2c(-c3ccc(-c4ccc(-c5nc(-c6ccccc6)nc(-c6ccc(-c7cccnc7)cc6)n5)cc4)cc3)cccc21 ; c1ccc(-c2nc(-c3ccc(-c4ccc(-c5cccc6c5-c5ccccc5C65c6ccccc6-c6ccccc65)cc4)cc3)nc(-c3ccc(-c4cccnc4)cc3)n2)cc1 ; c1ccc(-c2nc(-c3ccc(-c4ccc(-c5cccc6c5-c5ccccc5C6(c5ccccc5)c5ccccc5)cc4)cc3)nc(-c3ccc(-c4cccnc4)cc3)n2)cc1]

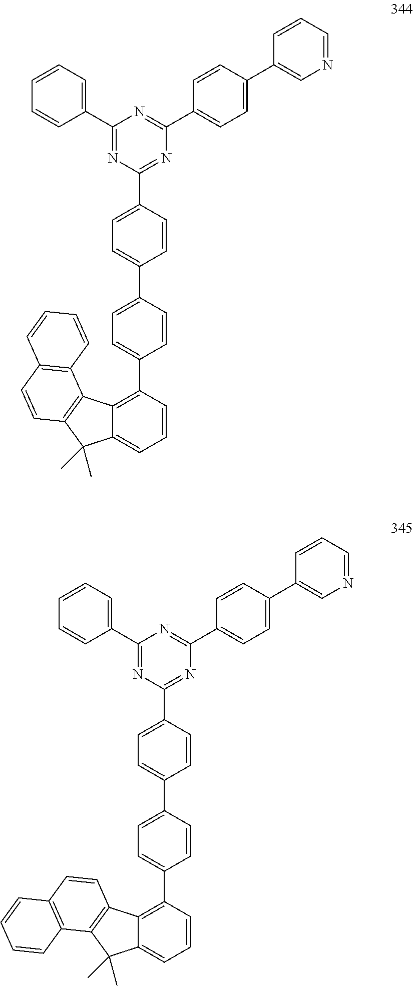 [SMILES: CC1(C)c2cccc(-c3ccc(-c4ccc(-c5nc(-c6ccccc6)nc(-c6ccc(-c7cccnc7)cc6)n5)cc4)cc3)c2-c2c1ccc1ccccc21 ; CC1(C)c2cccc(-c3ccc(-c4ccc(-c5nc(-c6ccccc6)nc(-c6ccc(-c7cccnc7)cc6)n5)cc4)cc3)c2-c2ccc3ccccc3c21]

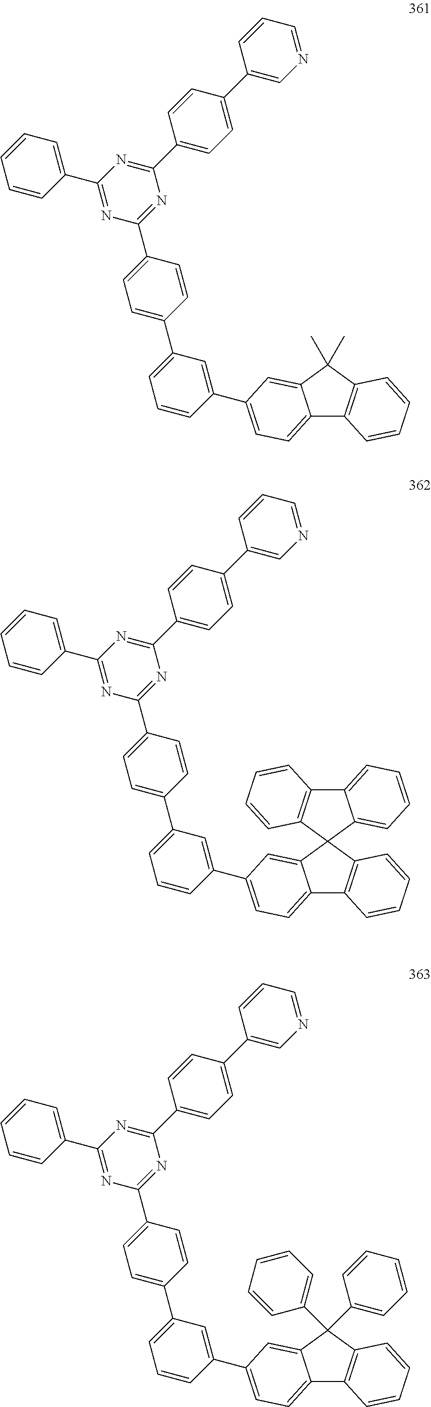 [SMILES: CC1(C)c2ccccc2-c2ccc(-c3cccc(-c4ccc(-c5nc(-c6ccccc6)nc(-c6ccc(-c7cccnc7)cc6)n5)cc4)c3)cc21 ; c1ccc(-c2nc(-c3ccc(-c4cccnc4)cc3)nc(-c3ccc(-c4cccc(-c5ccc6c(c5)C5(c7ccccc7-c7ccccc75)c5ccccc5-6)c4)cc3)n2)cc1 ; c1ccc(-c2nc(-c3ccc(-c4cccnc4)cc3)nc(-c3ccc(-c4cccc(-c5ccc6c(c5)C(c5ccccc5)(c5ccccc5)c5ccccc5-6)c4)cc3)n2)cc1]

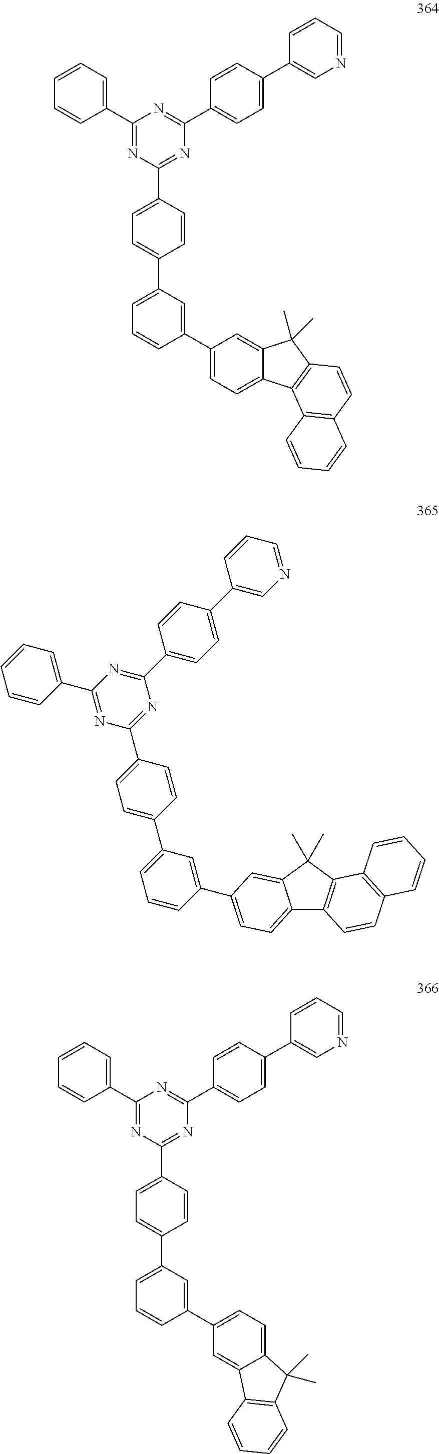 [SMILES: CC1(C)c2cc(-c3cccc(-c4ccc(-c5nc(-c6ccccc6)nc(-c6ccc(-c7cccnc7)cc6)n5)cc4)c3)ccc2-c2c1ccc1ccccc21 ; CC1(C)c2cc(-c3cccc(-c4ccc(-c5nc(-c6ccccc6)nc(-c6ccc(-c7cccnc7)cc6)n5)cc4)c3)ccc2-c2ccc3ccccc3c21 ; CC1(C)c2ccccc2-c2cc(-c3cccc(-c4ccc(-c5nc(-c6ccccc6)nc(-c6ccc(-c7cccnc7)cc6)n5)cc4)c3)ccc21]

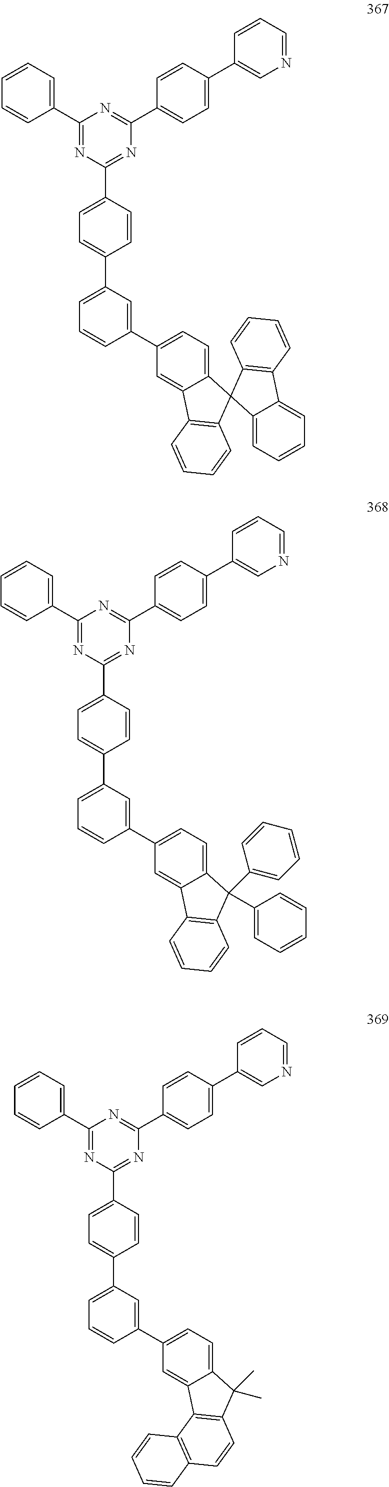 [SMILES: c1ccc(-c2nc(-c3ccc(-c4cccnc4)cc3)nc(-c3ccc(-c4cccc(-c5ccc6c(c5)-c5ccccc5C65c6ccccc6-c6ccccc65)c4)cc3)n2)cc1 ; c1ccc(-c2nc(-c3ccc(-c4cccnc4)cc3)nc(-c3ccc(-c4cccc(-c5ccc6c(c5)-c5ccccc5C6(c5ccccc5)c5ccccc5)c4)cc3)n2)cc1 ; CC1(C)c2ccc(-c3cccc(-c4ccc(-c5nc(-c6ccccc6)nc(-c6ccc(-c7cccnc7)cc6)n5)cc4)c3)cc2-c2c1ccc1ccccc21]

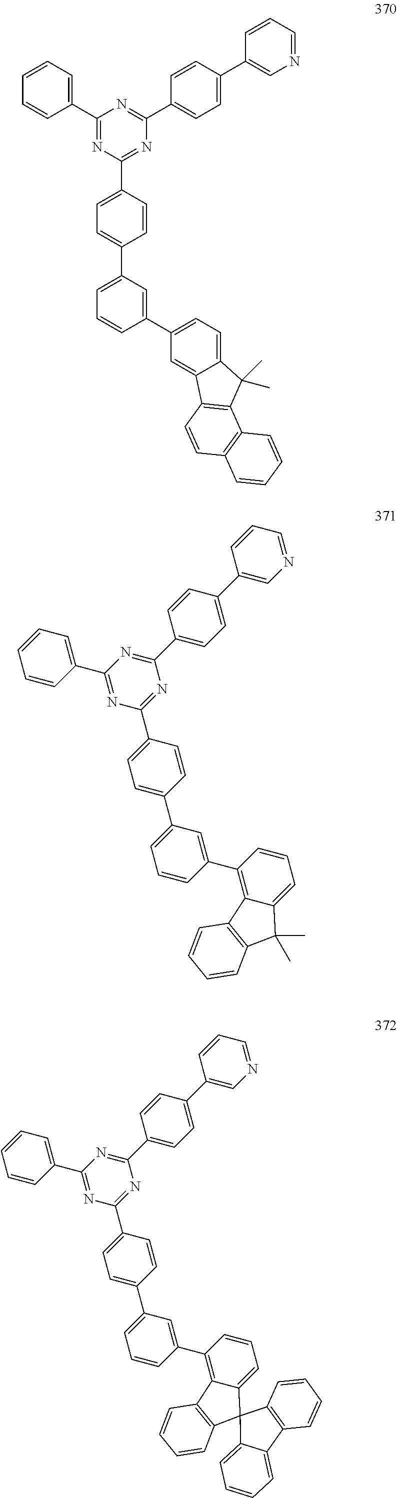 [SMILES: CC1(C)c2ccc(-c3cccc(-c4ccc(-c5nc(-c6ccccc6)nc(-c6ccc(-c7cccnc7)cc6)n5)cc4)c3)cc2-c2ccc3ccccc3c21 ; CC1(C)c2ccccc2-c2c(-c3cccc(-c4ccc(-c5nc(-c6ccccc6)nc(-c6ccc(-c7cccnc7)cc6)n5)cc4)c3)cccc21 ; c1ccc(-c2nc(-c3ccc(-c4cccnc4)cc3)nc(-c3ccc(-c4cccc(-c5cccc6c5-c5ccccc5C65c6ccccc6-c6ccccc65)c4)cc3)n2)cc1]

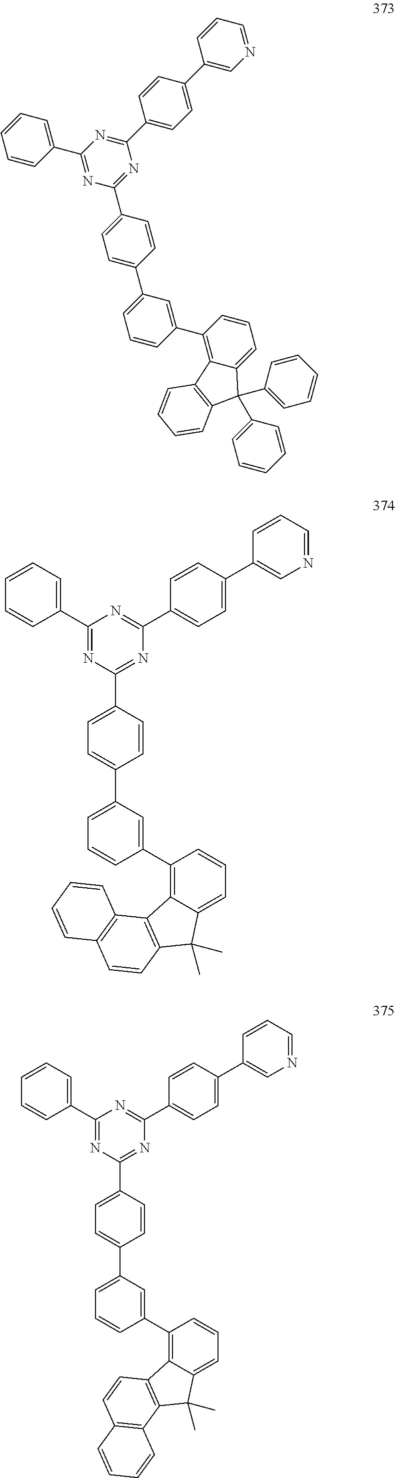 [SMILES: c1ccc(-c2nc(-c3ccc(-c4cccnc4)cc3)nc(-c3ccc(-c4cccc(-c5cccc6c5-c5ccccc5C6(c5ccccc5)c5ccccc5)c4)cc3)n2)cc1 ; CC1(C)c2cccc(-c3cccc(-c4ccc(-c5nc(-c6ccccc6)nc(-c6ccc(-c7cccnc7)cc6)n5)cc4)c3)c2-c2c1ccc1ccccc21 ; CC1(C)c2cccc(-c3cccc(-c4ccc(-c5nc(-c6ccccc6)nc(-c6ccc(-c7cccnc7)cc6)n5)cc4)c3)c2-c2ccc3ccccc3c21]

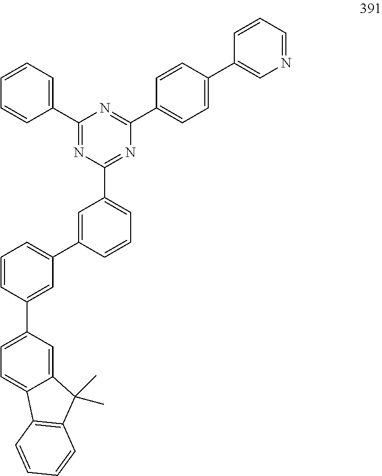 [SMILES: CC1(C)c2ccccc2-c2ccc(-c3cccc(-c4cccc(-c5nc(-c6ccccc6)nc(-c6ccc(-c7cccnc7)cc6)n5)c4)c3)cc21]

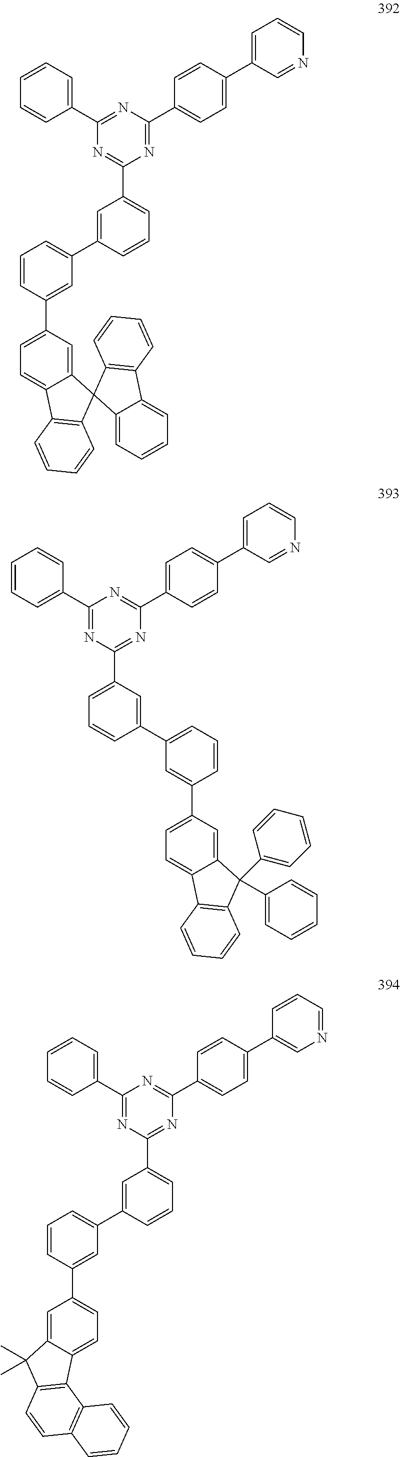 [SMILES: c1ccc(-c2nc(-c3ccc(-c4cccnc4)cc3)nc(-c3cccc(-c4cccc(-c5ccc6c(c5)C5(c7ccccc7-c7ccccc75)c5ccccc5-6)c4)c3)n2)cc1 ; c1ccc(-c2nc(-c3ccc(-c4cccnc4)cc3)nc(-c3cccc(-c4cccc(-c5ccc6c(c5)C(c5ccccc5)(c5ccccc5)c5ccccc5-6)c4)c3)n2)cc1 ; CC1(C)c2cc(-c3cccc(-c4cccc(-c5nc(-c6ccccc6)nc(-c6ccc(-c7cccnc7)cc6)n5)c4)c3)ccc2-c2c1ccc1ccccc21]

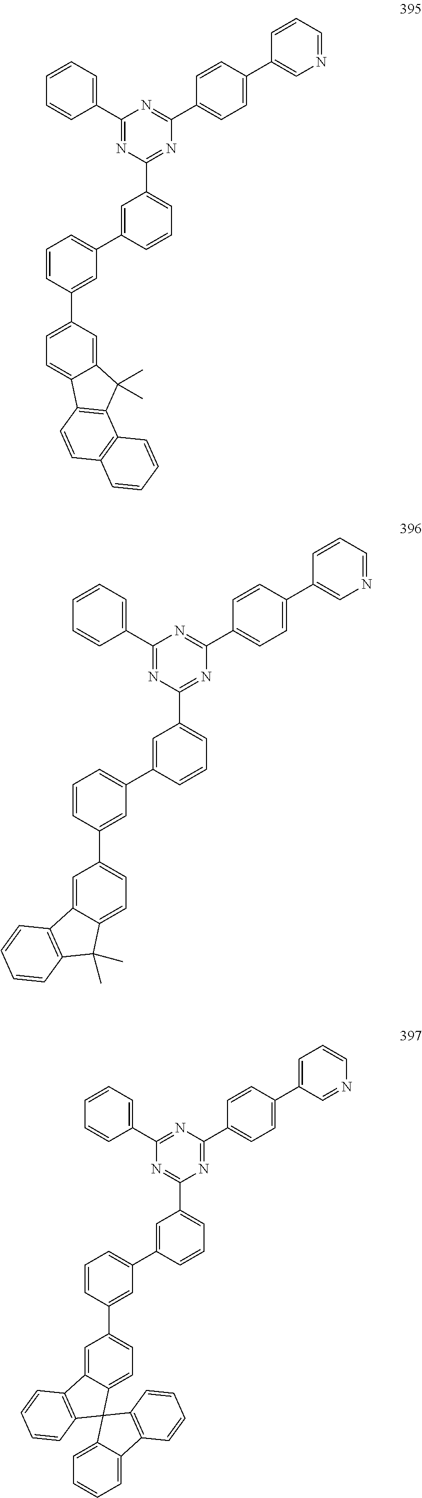 [SMILES: CC1(C)c2cc(-c3cccc(-c4cccc(-c5nc(-c6ccccc6)nc(-c6ccc(-c7cccnc7)cc6)n5)c4)c3)ccc2-c2ccc3ccccc3c21 ; CC1(C)c2ccccc2-c2cc(-c3cccc(-c4cccc(-c5nc(-c6ccccc6)nc(-c6ccc(-c7cccnc7)cc6)n5)c4)c3)ccc21 ; c1ccc(-c2nc(-c3ccc(-c4cccnc4)cc3)nc(-c3cccc(-c4cccc(-c5ccc6c(c5)-c5ccccc5C65c6ccccc6-c6ccccc65)c4)c3)n2)cc1]

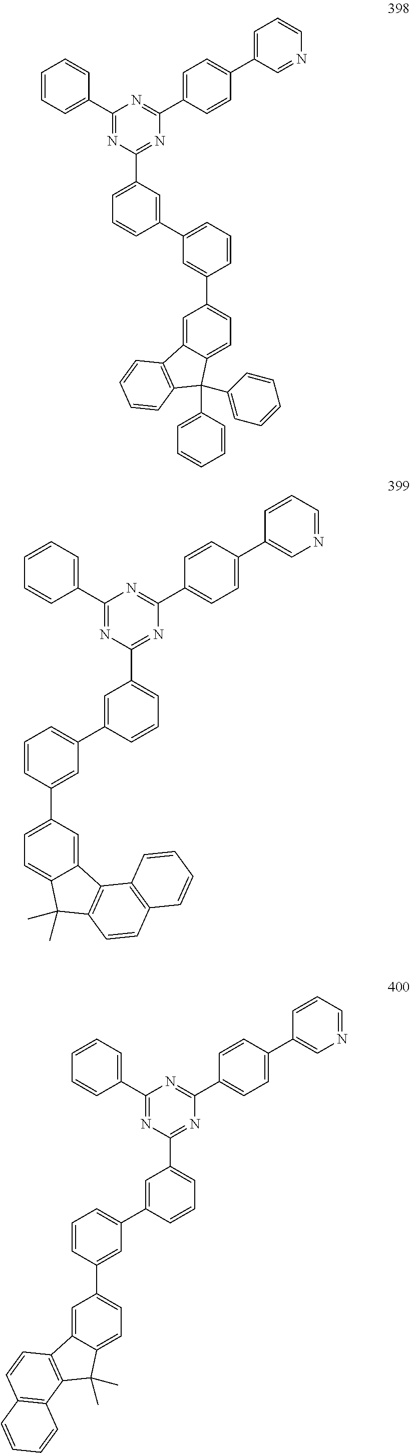 [SMILES: c1ccc(-c2nc(-c3ccc(-c4cccnc4)cc3)nc(-c3cccc(-c4cccc(-c5ccc6c(c5)-c5ccccc5C6(c5ccccc5)c5ccccc5)c4)c3)n2)cc1 ; CC1(C)c2ccc(-c3cccc(-c4cccc(-c5nc(-c6ccccc6)nc(-c6ccc(-c7cccnc7)cc6)n5)c4)c3)cc2-c2c1ccc1ccccc21 ; CC1(C)c2ccc(-c3cccc(-c4cccc(-c5nc(-c6ccccc6)nc(-c6ccc(-c7cccnc7)cc6)n5)c4)c3)cc2-c2ccc3ccccc3c21]

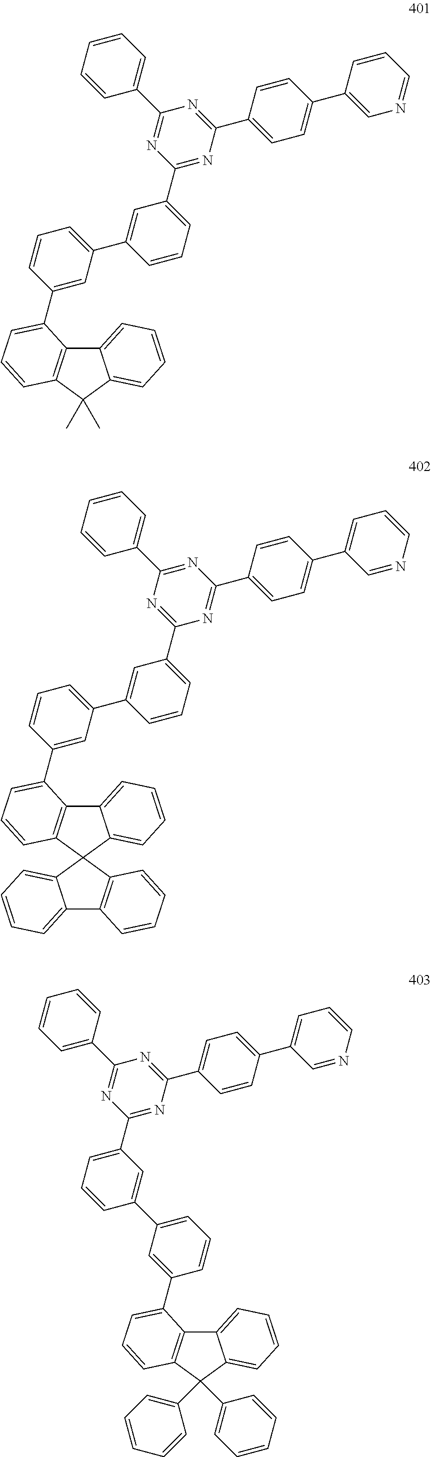 [SMILES: CC1(C)c2ccccc2-c2c(-c3cccc(-c4cccc(-c5nc(-c6ccccc6)nc(-c6ccc(-c7cccnc7)cc6)n5)c4)c3)cccc21 ; c1ccc(-c2nc(-c3ccc(-c4cccnc4)cc3)nc(-c3cccc(-c4cccc(-c5cccc6c5-c5ccccc5C65c6ccccc6-c6ccccc65)c4)c3)n2)cc1 ; c1ccc(-c2nc(-c3ccc(-c4cccnc4)cc3)nc(-c3cccc(-c4cccc(-c5cccc6c5-c5ccccc5C6(c5ccccc5)c5ccccc5)c4)c3)n2)cc1]

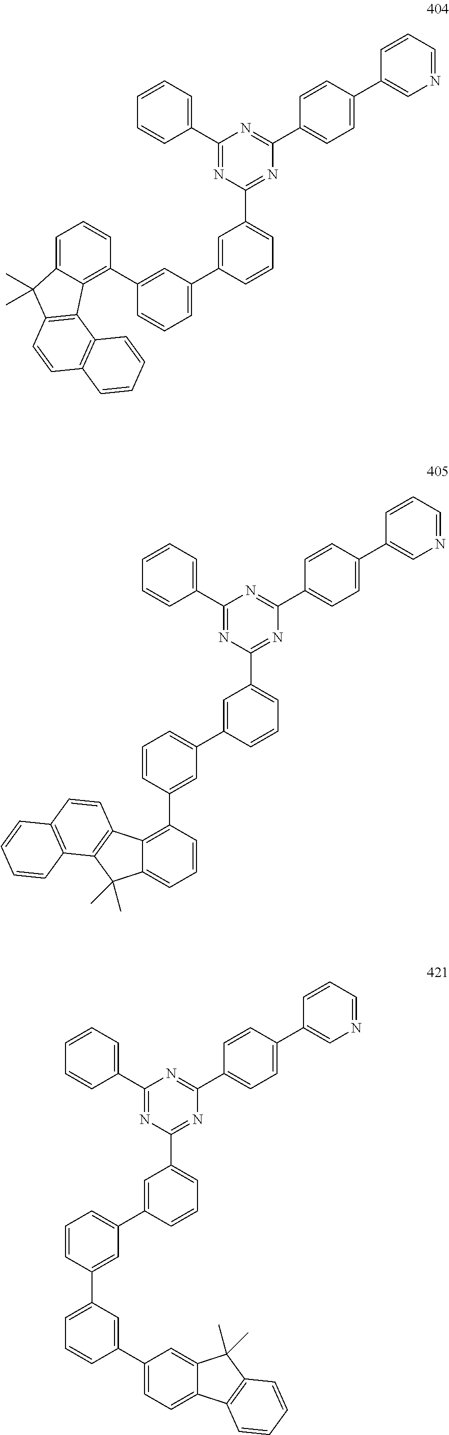 [SMILES: CC1(C)c2cccc(-c3cccc(-c4cccc(-c5nc(-c6ccccc6)nc(-c6ccc(-c7cccnc7)cc6)n5)c4)c3)c2-c2c1ccc1ccccc21 ; CC1(C)c2cccc(-c3cccc(-c4cccc(-c5nc(-c6ccccc6)nc(-c6ccc(-c7cccnc7)cc6)n5)c4)c3)c2-c2ccc3ccccc3c21 ; CC1(C)c2ccccc2-c2ccc(-c3cccc(-c4cccc(-c5cccc(-c6nc(-c7ccccc7)nc(-c7ccc(-c8cccnc8)cc7)n6)c5)c4)c3)cc21]

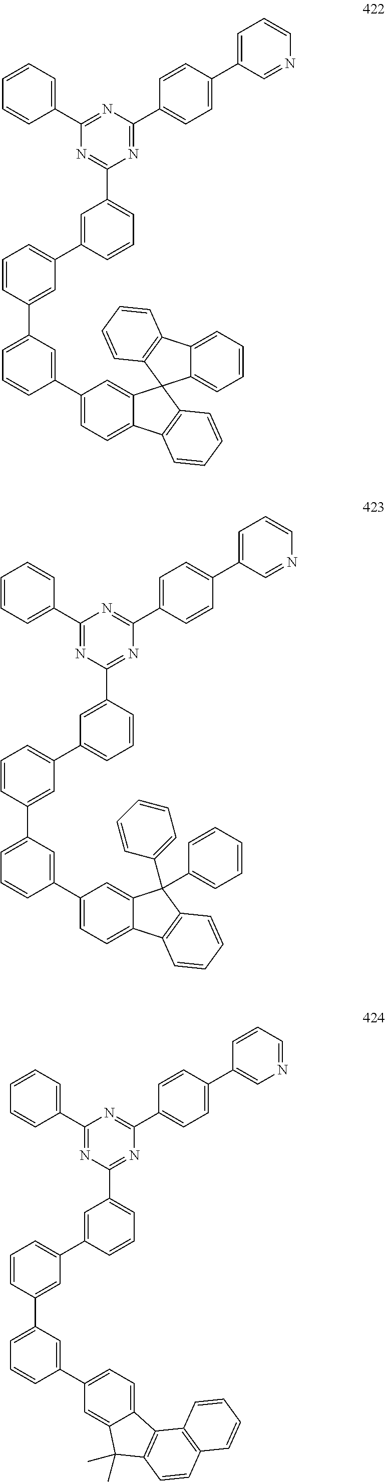 [SMILES: c1ccc(-c2nc(-c3ccc(-c4cccnc4)cc3)nc(-c3cccc(-c4cccc(-c5cccc(-c6ccc7c(c6)C6(c8ccccc8-c8ccccc86)c6ccccc6-7)c5)c4)c3)n2)cc1 ; c1ccc(-c2nc(-c3ccc(-c4cccnc4)cc3)nc(-c3cccc(-c4cccc(-c5cccc(-c6ccc7c(c6)C(c6ccccc6)(c6ccccc6)c6ccccc6-7)c5)c4)c3)n2)cc1 ; CC1(C)c2cc(-c3cccc(-c4cccc(-c5cccc(-c6nc(-c7ccccc7)nc(-c7ccc(-c8cccnc8)cc7)n6)c5)c4)c3)ccc2-c2c1ccc1ccccc21]

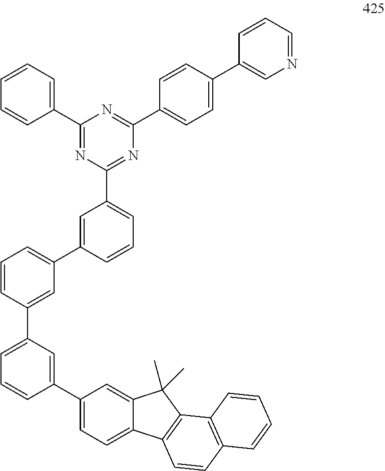 [SMILES: CC1(C)c2cc(-c3cccc(-c4cccc(-c5cccc(-c6nc(-c7ccccc7)nc(-c7ccc(-c8cccnc8)cc7)n6)c5)c4)c3)ccc2-c2ccc3ccccc3c21]

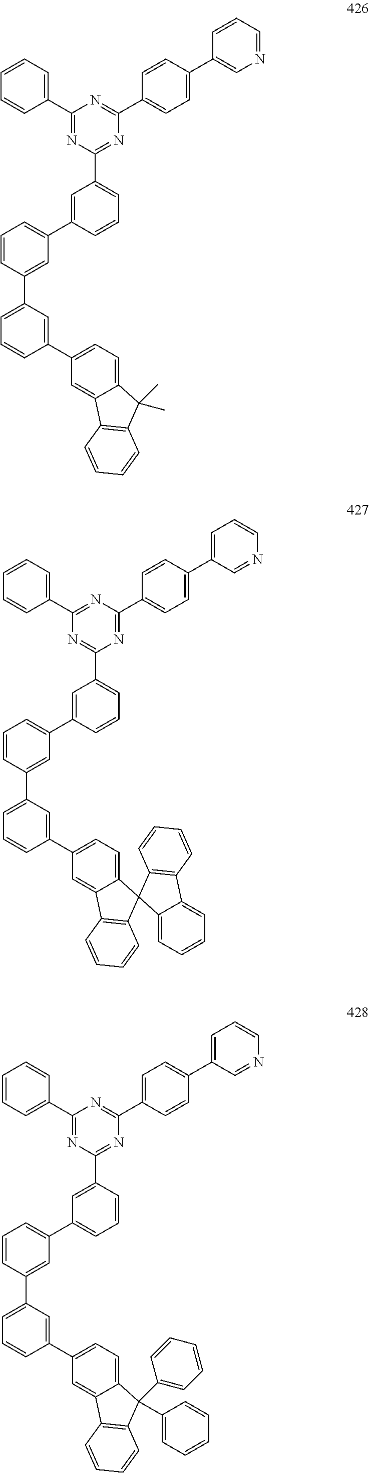 [SMILES: CC1(C)c2ccccc2-c2cc(-c3cccc(-c4cccc(-c5cccc(-c6nc(-c7ccccc7)nc(-c7ccc(-c8cccnc8)cc7)n6)c5)c4)c3)ccc21 ; c1ccc(-c2nc(-c3ccc(-c4cccnc4)cc3)nc(-c3cccc(-c4cccc(-c5cccc(-c6ccc7c(c6)-c6ccccc6C76c7ccccc7-c7ccccc76)c5)c4)c3)n2)cc1 ; c1ccc(-c2nc(-c3ccc(-c4cccnc4)cc3)nc(-c3cccc(-c4cccc(-c5cccc(-c6ccc7c(c6)-c6ccccc6C7(c6ccccc6)c6ccccc6)c5)c4)c3)n2)cc1]

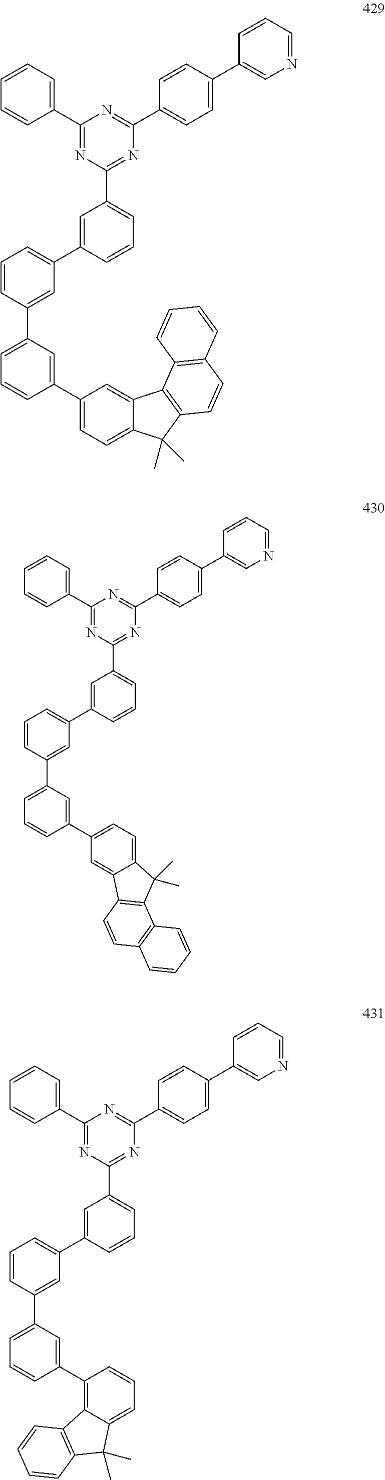 [SMILES: CC1(C)c2ccc(-c3cccc(-c4cccc(-c5cccc(-c6nc(-c7ccccc7)nc(-c7ccc(-c8cccnc8)cc7)n6)c5)c4)c3)cc2-c2c1ccc1ccccc21 ; CC1(C)c2ccc(-c3cccc(-c4cccc(-c5cccc(-c6nc(-c7ccccc7)nc(-c7ccc(-c8cccnc8)cc7)n6)c5)c4)c3)cc2-c2ccc3ccccc3c21 ; CC1(C)c2ccccc2-c2c(-c3cccc(-c4cccc(-c5cccc(-c6nc(-c7ccccc7)nc(-c7ccc(-c8cccnc8)cc7)n6)c5)c4)c3)cccc21]

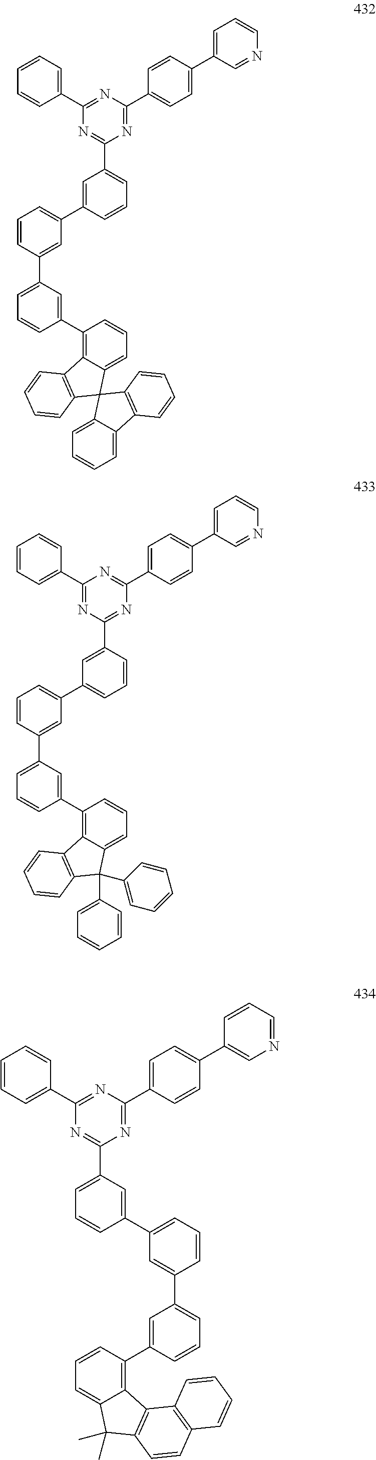 [SMILES: c1ccc(-c2nc(-c3ccc(-c4cccnc4)cc3)nc(-c3cccc(-c4cccc(-c5cccc(-c6cccc7c6-c6ccccc6C76c7ccccc7-c7ccccc76)c5)c4)c3)n2)cc1 ; c1ccc(-c2nc(-c3ccc(-c4cccnc4)cc3)nc(-c3cccc(-c4cccc(-c5cccc(-c6cccc7c6-c6ccccc6C7(c6ccccc6)c6ccccc6)c5)c4)c3)n2)cc1 ; CC1(C)c2cccc(-c3cccc(-c4cccc(-c5cccc(-c6nc(-c7ccccc7)nc(-c7ccc(-c8cccnc8)cc7)n6)c5)c4)c3)c2-c2c1ccc1ccccc21]

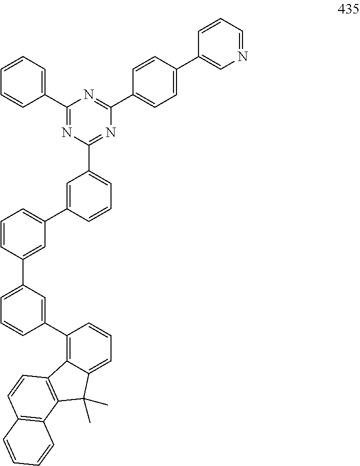 [SMILES: CC1(C)c2cccc(-c3cccc(-c4cccc(-c5cccc(-c6nc(-c7ccccc7)nc(-c7ccc(-c8cccnc8)cc7)n6)c5)c4)c3)c2-c2ccc3ccccc3c21]

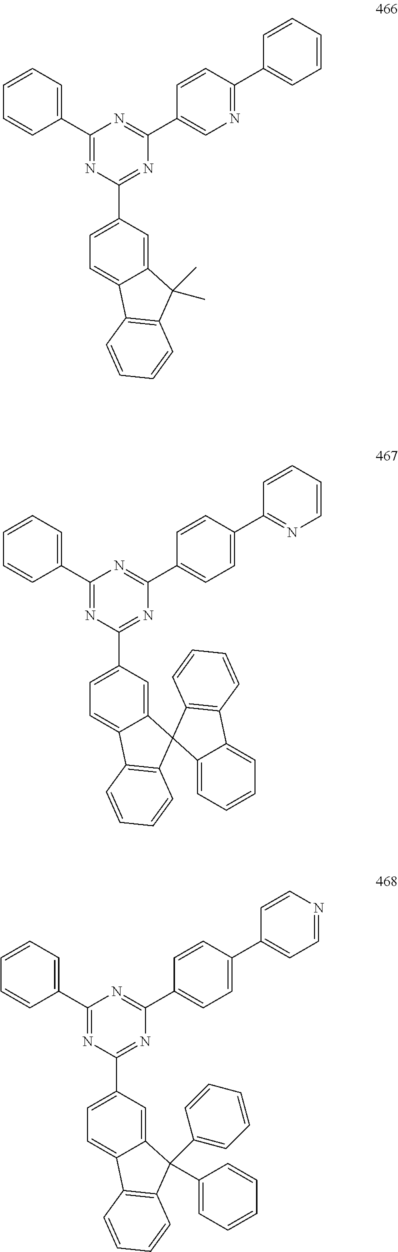 [SMILES: CC1(C)c2ccccc2-c2ccc(-c3nc(-c4ccccc4)nc(-c4ccc(-c5ccccc5)nc4)n3)cc21 ; c1ccc(-c2nc(-c3ccc(-c4ccccn4)cc3)nc(-c3ccc4c(c3)C3(c5ccccc5-c5ccccc53)c3ccccc3-4)n2)cc1 ; c1ccc(-c2nc(-c3ccc(-c4ccncc4)cc3)nc(-c3ccc4c(c3)C(c3ccccc3)(c3ccccc3)c3ccccc3-4)n2)cc1]

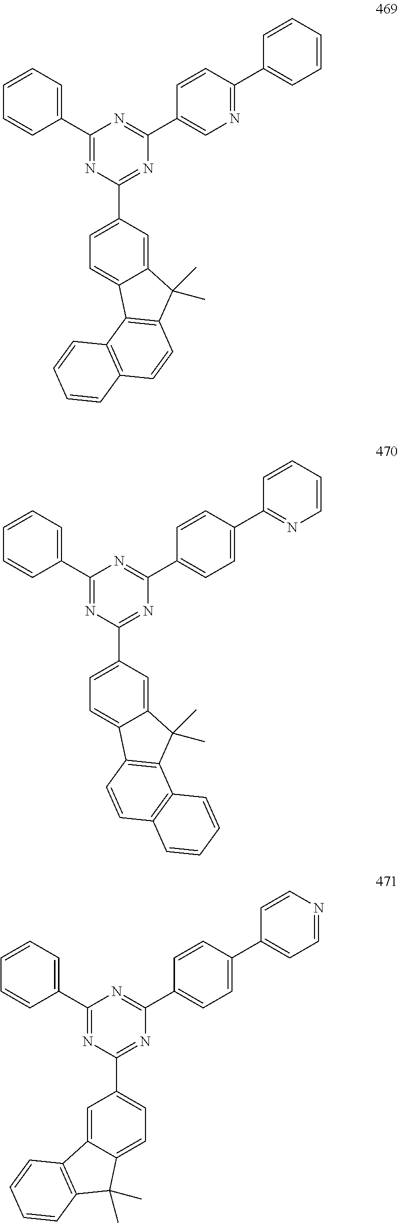 [SMILES: CC1(C)c2cc(-c3nc(-c4ccccc4)nc(-c4ccc(-c5ccccc5)nc4)n3)ccc2-c2c1ccc1ccccc21 ; CC1(C)c2cc(-c3nc(-c4ccccc4)nc(-c4ccc(-c5ccccn5)cc4)n3)ccc2-c2ccc3ccccc3c21 ; CC1(C)c2ccccc2-c2cc(-c3nc(-c4ccccc4)nc(-c4ccc(-c5ccncc5)cc4)n3)ccc21]

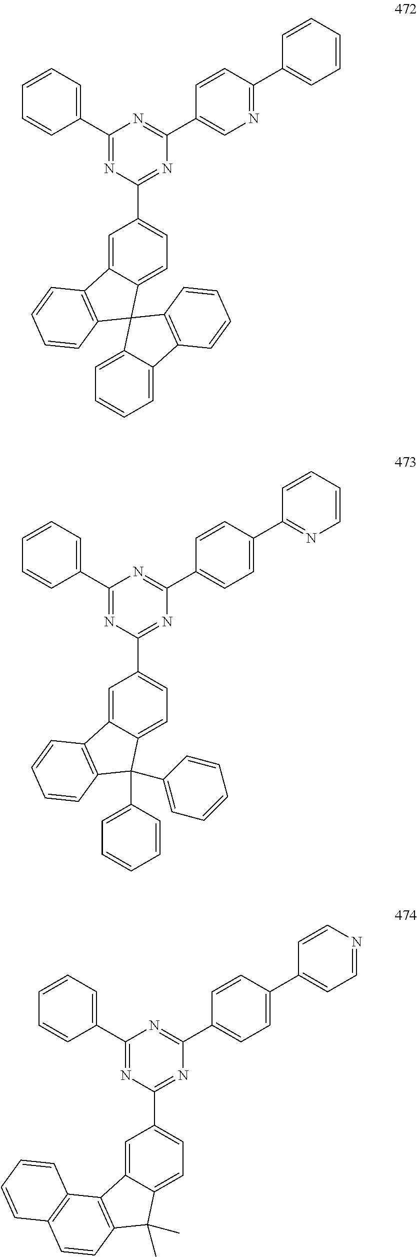 [SMILES: c1ccc(-c2ccc(-c3nc(-c4ccccc4)nc(-c4ccc5c(c4)-c4ccccc4C54c5ccccc5-c5ccccc54)n3)cn2)cc1 ; c1ccc(-c2nc(-c3ccc(-c4ccccn4)cc3)nc(-c3ccc4c(c3)-c3ccccc3C4(c3ccccc3)c3ccccc3)n2)cc1 ; CC1(C)c2ccc(-c3nc(-c4ccccc4)nc(-c4ccc(-c5ccncc5)cc4)n3)cc2-c2c1ccc1ccccc21]

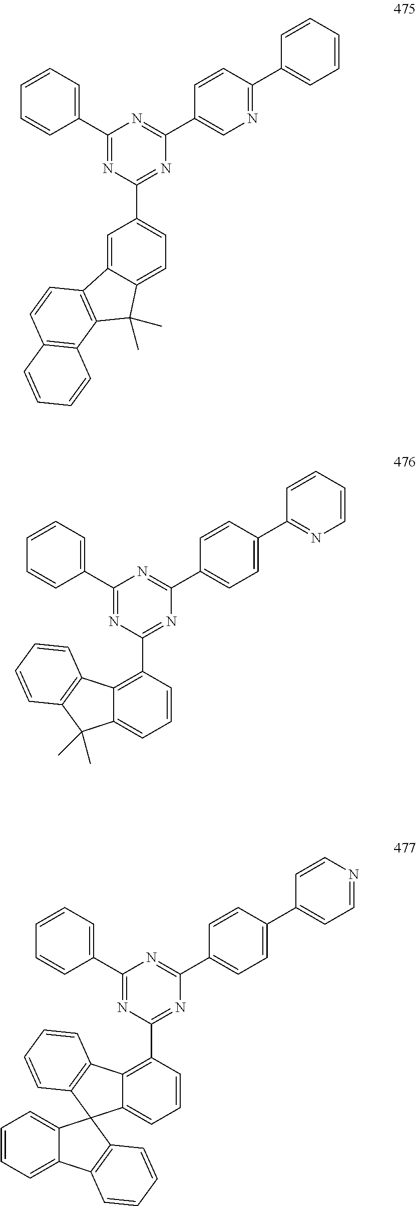 [SMILES: CC1(C)c2ccc(-c3nc(-c4ccccc4)nc(-c4ccc(-c5ccccc5)nc4)n3)cc2-c2ccc3ccccc3c21 ; CC1(C)c2ccccc2-c2c(-c3nc(-c4ccccc4)nc(-c4ccc(-c5ccccn5)cc4)n3)cccc21 ; c1ccc(-c2nc(-c3ccc(-c4ccncc4)cc3)nc(-c3cccc4c3-c3ccccc3C43c4ccccc4-c4ccccc43)n2)cc1]

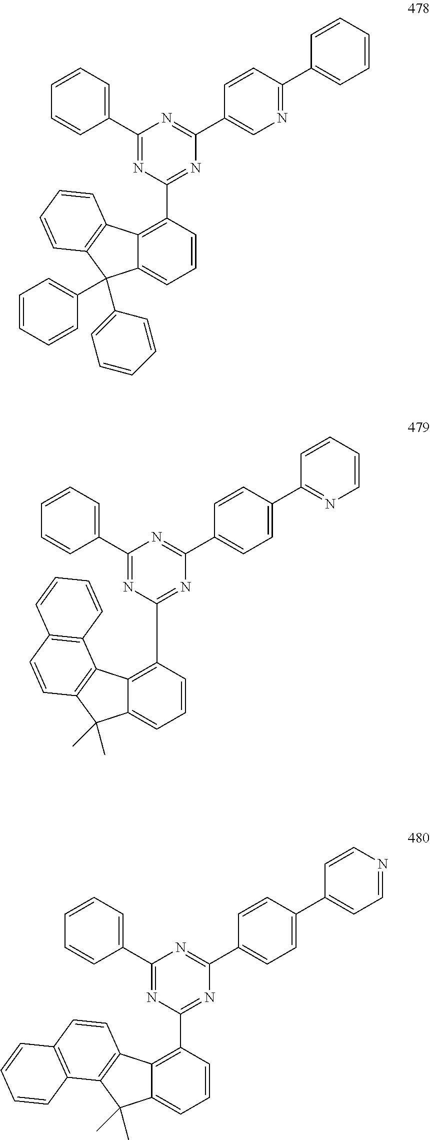 [SMILES: c1ccc(-c2ccc(-c3nc(-c4ccccc4)nc(-c4cccc5c4-c4ccccc4C5(c4ccccc4)c4ccccc4)n3)cn2)cc1 ; CC1(C)c2cccc(-c3nc(-c4ccccc4)nc(-c4ccc(-c5ccccn5)cc4)n3)c2-c2c1ccc1ccccc21 ; CC1(C)c2cccc(-c3nc(-c4ccccc4)nc(-c4ccc(-c5ccncc5)cc4)n3)c2-c2ccc3ccccc3c21]

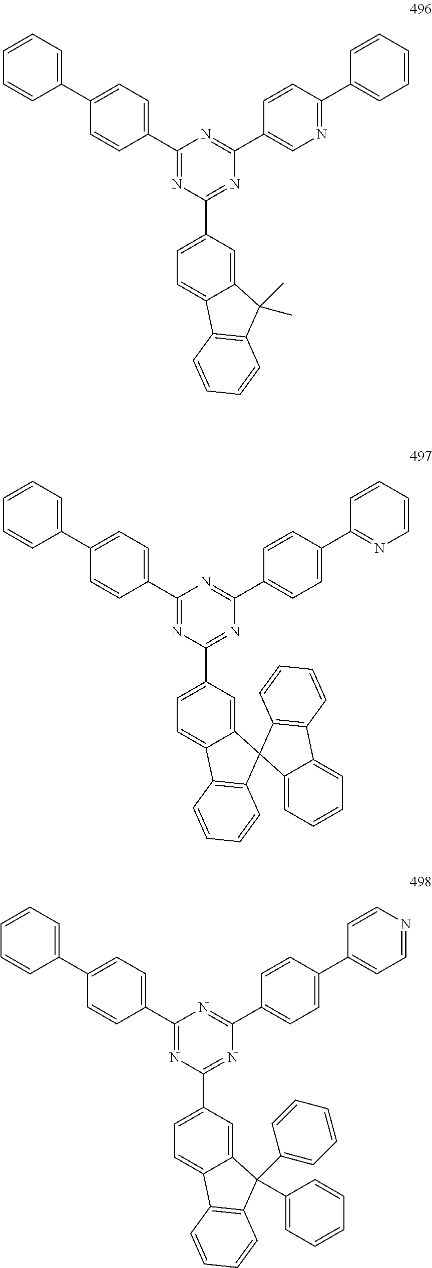 [SMILES: CC1(C)c2ccccc2-c2ccc(-c3nc(-c4ccc(-c5ccccc5)cc4)nc(-c4ccc(-c5ccccc5)nc4)n3)cc21 ; c1ccc(-c2ccc(-c3nc(-c4ccc(-c5ccccn5)cc4)nc(-c4ccc5c(c4)C4(c6ccccc6-c6ccccc64)c4ccccc4-5)n3)cc2)cc1 ; c1ccc(-c2ccc(-c3nc(-c4ccc(-c5ccncc5)cc4)nc(-c4ccc5c(c4)C(c4ccccc4)(c4ccccc4)c4ccccc4-5)n3)cc2)cc1]

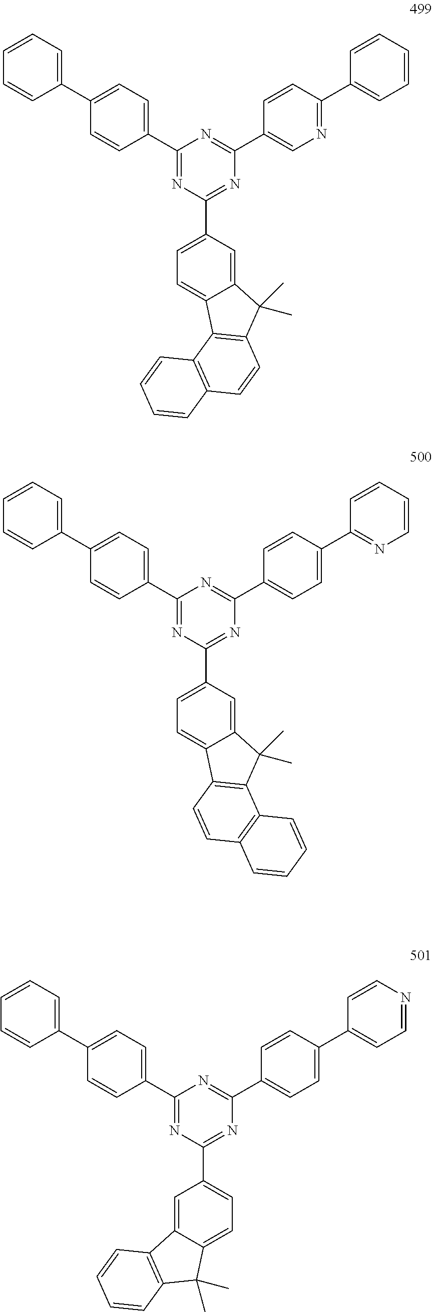 [SMILES: CC1(C)c2cc(-c3nc(-c4ccc(-c5ccccc5)cc4)nc(-c4ccc(-c5ccccc5)nc4)n3)ccc2-c2c1ccc1ccccc21 ; CC1(C)c2cc(-c3nc(-c4ccc(-c5ccccc5)cc4)nc(-c4ccc(-c5ccccn5)cc4)n3)ccc2-c2ccc3ccccc3c21 ; CC1(C)c2ccccc2-c2cc(-c3nc(-c4ccc(-c5ccccc5)cc4)nc(-c4ccc(-c5ccncc5)cc4)n3)ccc21]

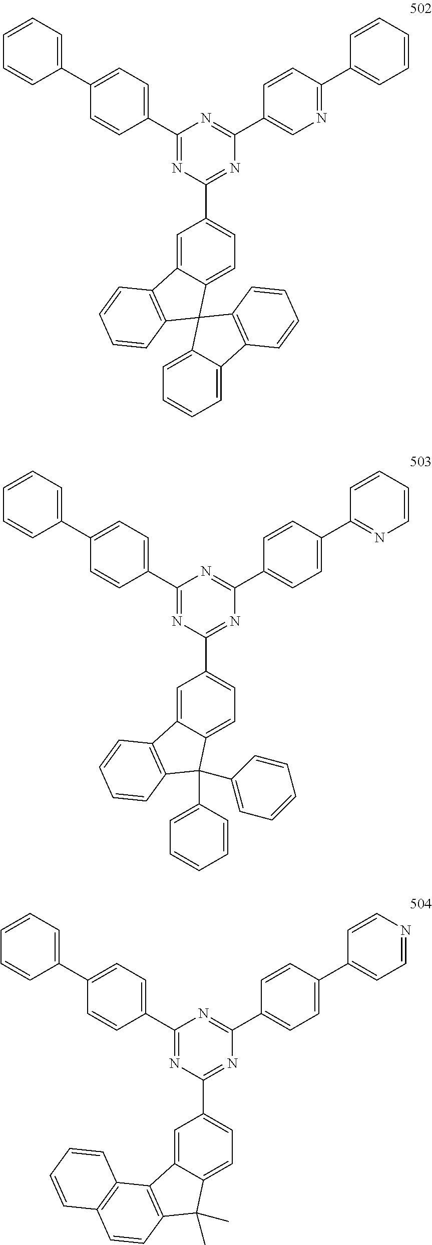 [SMILES: c1ccc(-c2ccc(-c3nc(-c4ccc(-c5ccccc5)nc4)nc(-c4ccc5c(c4)-c4ccccc4C54c5ccccc5-c5ccccc54)n3)cc2)cc1 ; c1ccc(-c2ccc(-c3nc(-c4ccc(-c5ccccn5)cc4)nc(-c4ccc5c(c4)-c4ccccc4C5(c4ccccc4)c4ccccc4)n3)cc2)cc1 ; CC1(C)c2ccc(-c3nc(-c4ccc(-c5ccccc5)cc4)nc(-c4ccc(-c5ccncc5)cc4)n3)cc2-c2c1ccc1ccccc21]

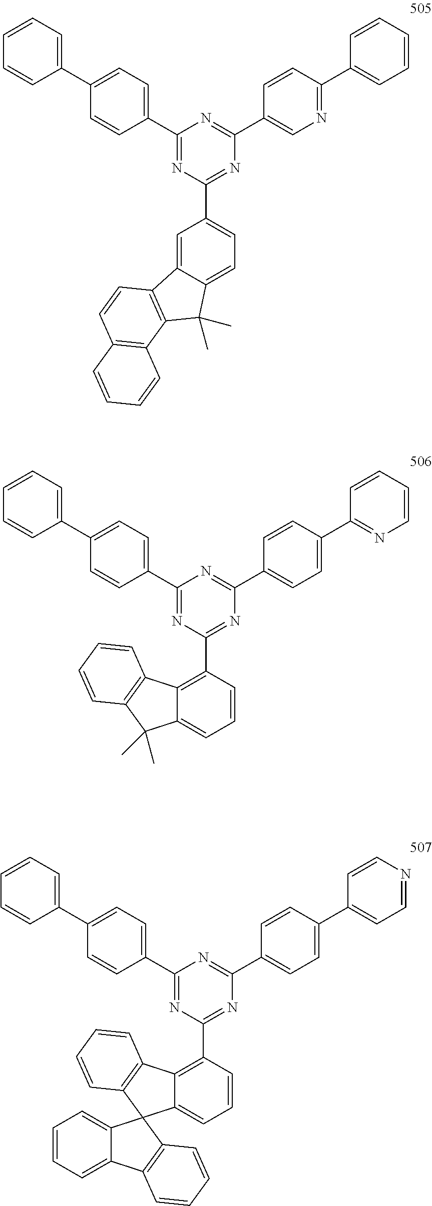 [SMILES: CC1(C)c2ccc(-c3nc(-c4ccc(-c5ccccc5)cc4)nc(-c4ccc(-c5ccccc5)nc4)n3)cc2-c2ccc3ccccc3c21 ; CC1(C)c2ccccc2-c2c(-c3nc(-c4ccc(-c5ccccc5)cc4)nc(-c4ccc(-c5ccccn5)cc4)n3)cccc21 ; c1ccc(-c2ccc(-c3nc(-c4ccc(-c5ccncc5)cc4)nc(-c4cccc5c4-c4ccccc4C54c5ccccc5-c5ccccc54)n3)cc2)cc1]

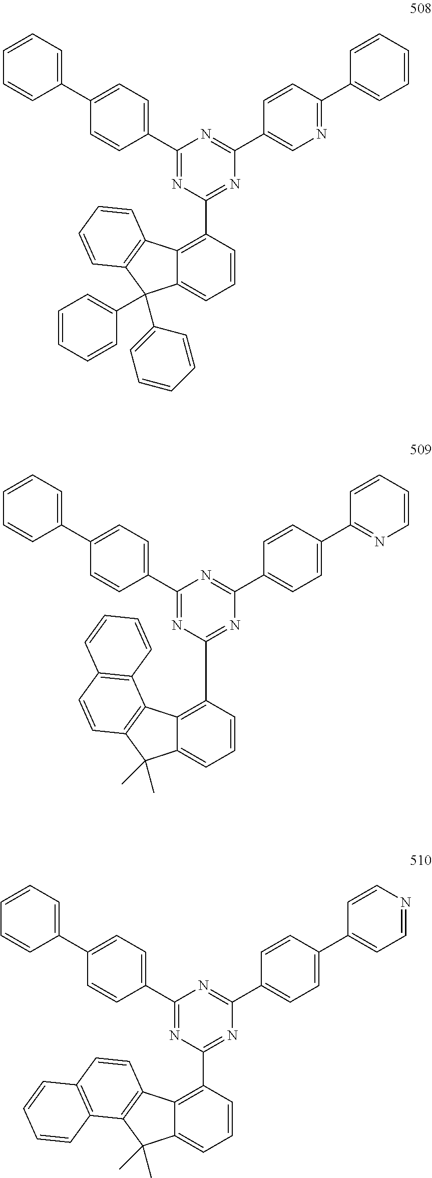 [SMILES: c1ccc(-c2ccc(-c3nc(-c4ccc(-c5ccccc5)nc4)nc(-c4cccc5c4-c4ccccc4C5(c4ccccc4)c4ccccc4)n3)cc2)cc1 ; CC1(C)c2cccc(-c3nc(-c4ccc(-c5ccccc5)cc4)nc(-c4ccc(-c5ccccn5)cc4)n3)c2-c2c1ccc1ccccc21 ; CC1(C)c2cccc(-c3nc(-c4ccc(-c5ccccc5)cc4)nc(-c4ccc(-c5ccncc5)cc4)n3)c2-c2ccc3ccccc3c21]

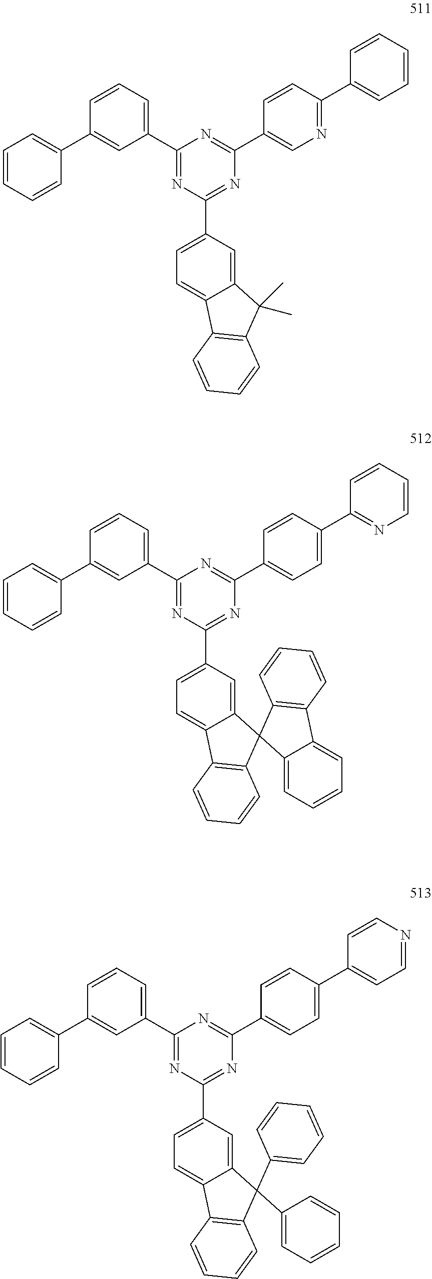 [SMILES: CC1(C)c2ccccc2-c2ccc(-c3nc(-c4ccc(-c5ccccc5)nc4)nc(-c4cccc(-c5ccccc5)c4)n3)cc21 ; c1ccc(-c2cccc(-c3nc(-c4ccc(-c5ccccn5)cc4)nc(-c4ccc5c(c4)C4(c6ccccc6-c6ccccc64)c4ccccc4-5)n3)c2)cc1 ; c1ccc(-c2cccc(-c3nc(-c4ccc(-c5ccncc5)cc4)nc(-c4ccc5c(c4)C(c4ccccc4)(c4ccccc4)c4ccccc4-5)n3)c2)cc1]

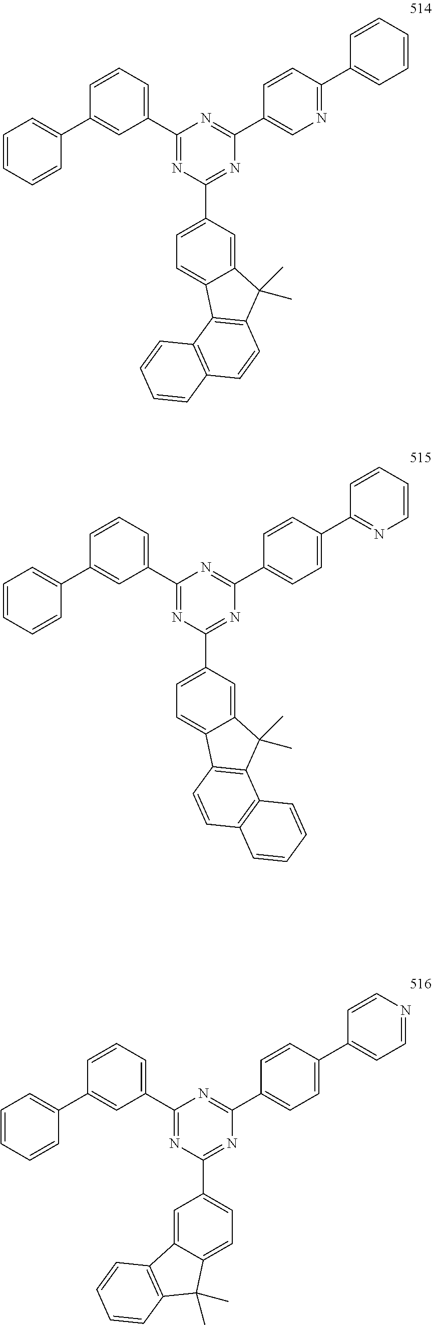 [SMILES: CC1(C)c2cc(-c3nc(-c4ccc(-c5ccccc5)nc4)nc(-c4cccc(-c5ccccc5)c4)n3)ccc2-c2c1ccc1ccccc21 ; CC1(C)c2cc(-c3nc(-c4ccc(-c5ccccn5)cc4)nc(-c4cccc(-c5ccccc5)c4)n3)ccc2-c2ccc3ccccc3c21 ; CC1(C)c2ccccc2-c2cc(-c3nc(-c4ccc(-c5ccncc5)cc4)nc(-c4cccc(-c5ccccc5)c4)n3)ccc21]

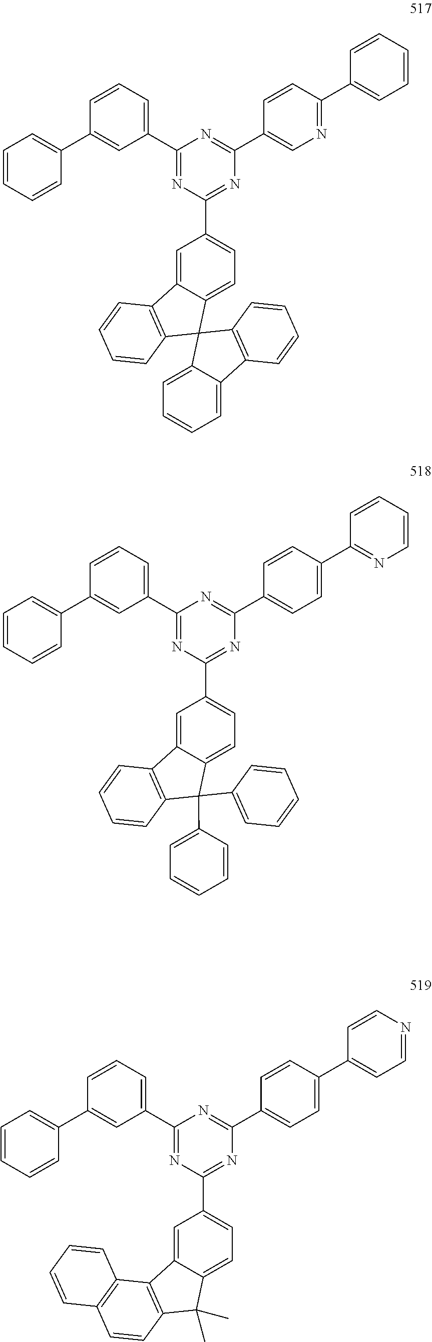 [SMILES: c1ccc(-c2cccc(-c3nc(-c4ccc(-c5ccccc5)nc4)nc(-c4ccc5c(c4)-c4ccccc4C54c5ccccc5-c5ccccc54)n3)c2)cc1 ; c1ccc(-c2cccc(-c3nc(-c4ccc(-c5ccccn5)cc4)nc(-c4ccc5c(c4)-c4ccccc4C5(c4ccccc4)c4ccccc4)n3)c2)cc1 ; CC1(C)c2ccc(-c3nc(-c4ccc(-c5ccncc5)cc4)nc(-c4cccc(-c5ccccc5)c4)n3)cc2-c2c1ccc1ccccc21]

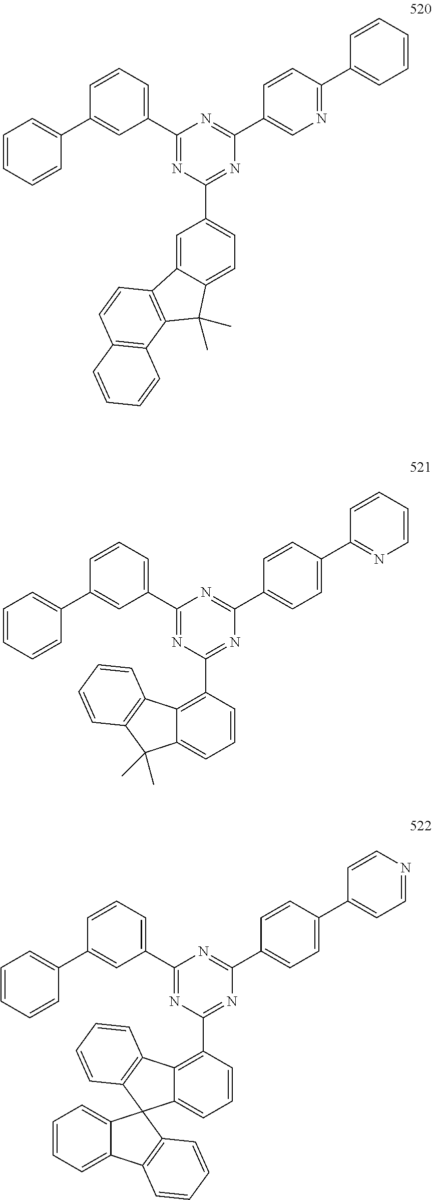 [SMILES: CC1(C)c2ccc(-c3nc(-c4ccc(-c5ccccc5)nc4)nc(-c4cccc(-c5ccccc5)c4)n3)cc2-c2ccc3ccccc3c21 ; CC1(C)c2ccccc2-c2c(-c3nc(-c4ccc(-c5ccccn5)cc4)nc(-c4cccc(-c5ccccc5)c4)n3)cccc21 ; c1ccc(-c2cccc(-c3nc(-c4ccc(-c5ccncc5)cc4)nc(-c4cccc5c4-c4ccccc4C54c5ccccc5-c5ccccc54)n3)c2)cc1]

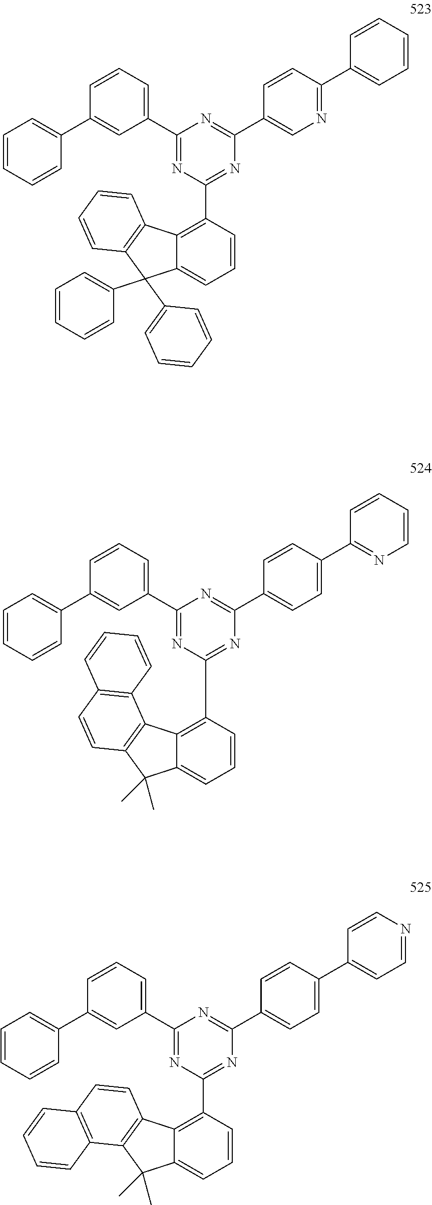 [SMILES: c1ccc(-c2cccc(-c3nc(-c4ccc(-c5ccccc5)nc4)nc(-c4cccc5c4-c4ccccc4C5(c4ccccc4)c4ccccc4)n3)c2)cc1 ; CC1(C)c2cccc(-c3nc(-c4ccc(-c5ccccn5)cc4)nc(-c4cccc(-c5ccccc5)c4)n3)c2-c2c1ccc1ccccc21 ; CC1(C)c2cccc(-c3nc(-c4ccc(-c5ccncc5)cc4)nc(-c4cccc(-c5ccccc5)c4)n3)c2-c2ccc3ccccc3c21]

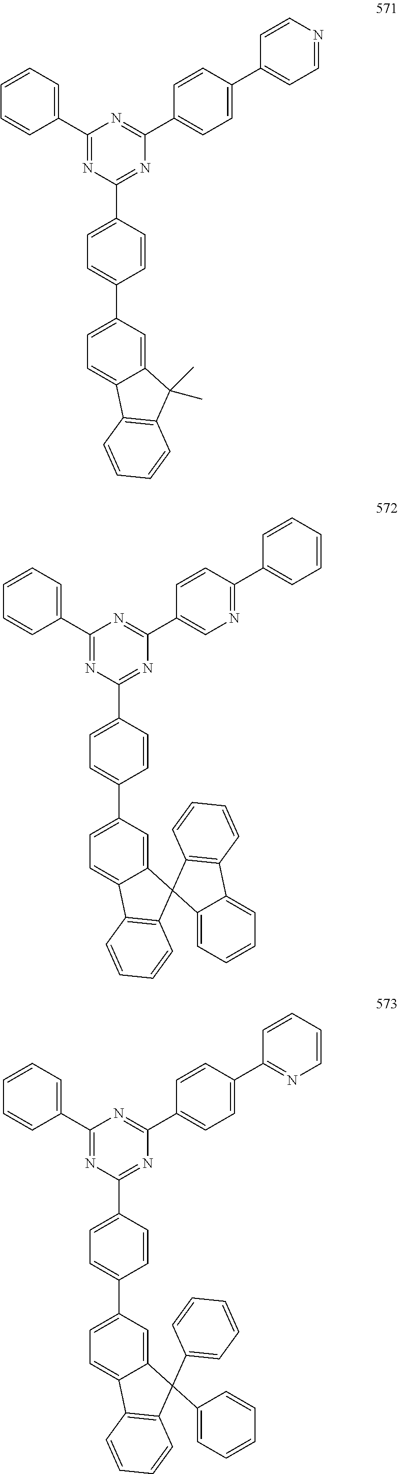 [SMILES: CC1(C)c2ccccc2-c2ccc(-c3ccc(-c4nc(-c5ccccc5)nc(-c5ccc(-c6ccncc6)cc5)n4)cc3)cc21 ; c1ccc(-c2ccc(-c3nc(-c4ccccc4)nc(-c4ccc(-c5ccc6c(c5)C5(c7ccccc7-c7ccccc75)c5ccccc5-6)cc4)n3)cn2)cc1 ; c1ccc(-c2nc(-c3ccc(-c4ccc5c(c4)C(c4ccccc4)(c4ccccc4)c4ccccc4-5)cc3)nc(-c3ccc(-c4ccccn4)cc3)n2)cc1]

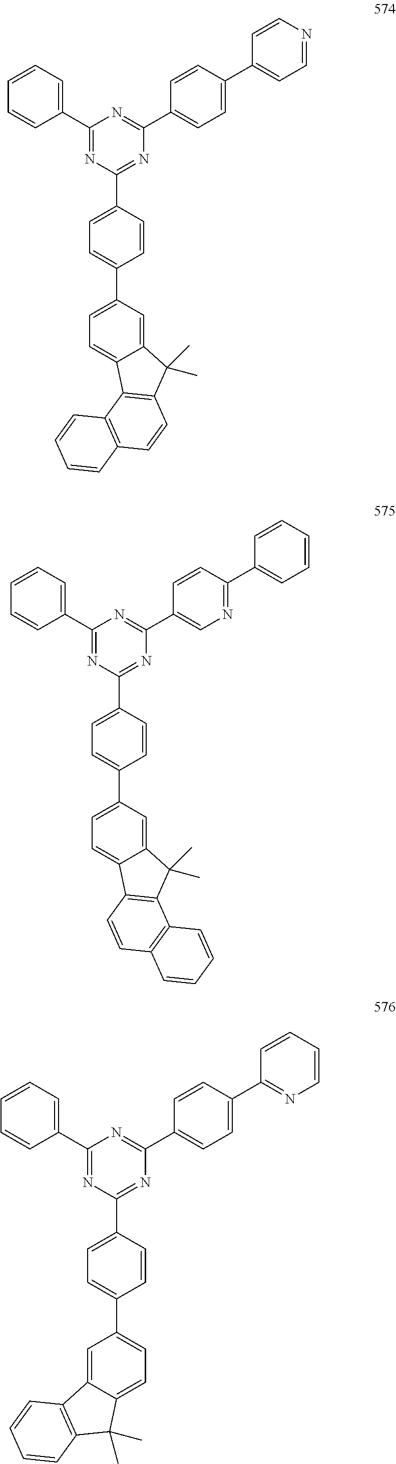 [SMILES: CC1(C)c2cc(-c3ccc(-c4nc(-c5ccccc5)nc(-c5ccc(-c6ccncc6)cc5)n4)cc3)ccc2-c2c1ccc1ccccc21 ; CC1(C)c2cc(-c3ccc(-c4nc(-c5ccccc5)nc(-c5ccc(-c6ccccc6)nc5)n4)cc3)ccc2-c2ccc3ccccc3c21 ; CC1(C)c2ccccc2-c2cc(-c3ccc(-c4nc(-c5ccccc5)nc(-c5ccc(-c6ccccn6)cc5)n4)cc3)ccc21]

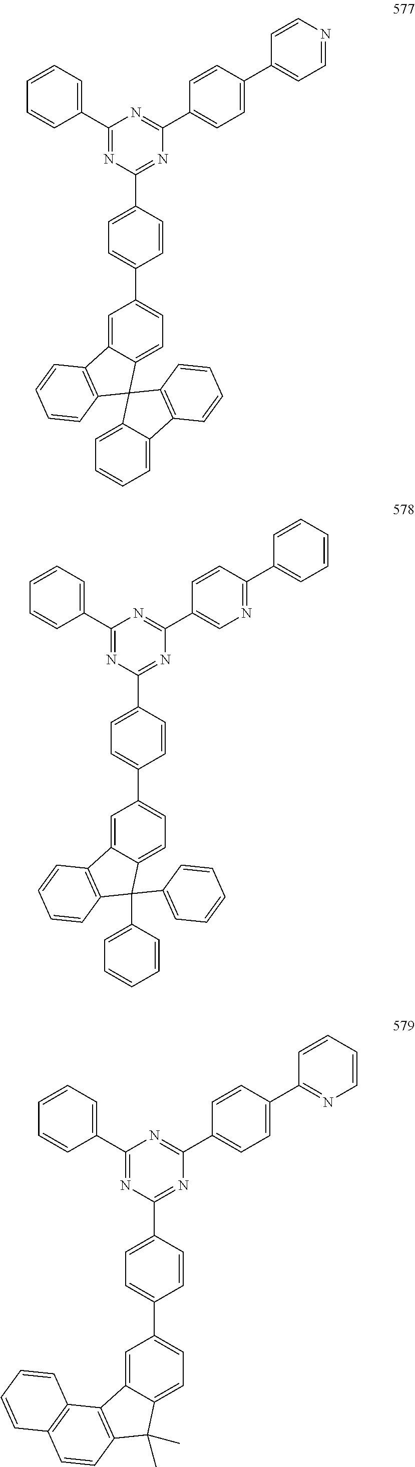 [SMILES: c1ccc(-c2nc(-c3ccc(-c4ccncc4)cc3)nc(-c3ccc(-c4ccc5c(c4)-c4ccccc4C54c5ccccc5-c5ccccc54)cc3)n2)cc1 ; c1ccc(-c2ccc(-c3nc(-c4ccccc4)nc(-c4ccc(-c5ccc6c(c5)-c5ccccc5C6(c5ccccc5)c5ccccc5)cc4)n3)cn2)cc1 ; CC1(C)c2ccc(-c3ccc(-c4nc(-c5ccccc5)nc(-c5ccc(-c6ccccn6)cc5)n4)cc3)cc2-c2c1ccc1ccccc21]

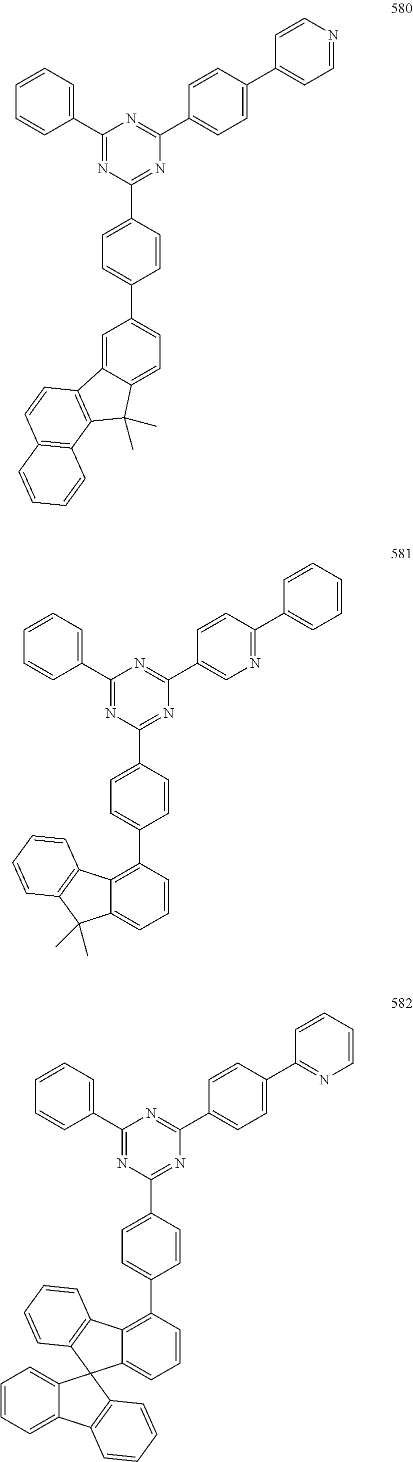 [SMILES: CC1(C)c2ccc(-c3ccc(-c4nc(-c5ccccc5)nc(-c5ccc(-c6ccncc6)cc5)n4)cc3)cc2-c2ccc3ccccc3c21 ; CC1(C)c2ccccc2-c2c(-c3ccc(-c4nc(-c5ccccc5)nc(-c5ccc(-c6ccccc6)nc5)n4)cc3)cccc21 ; c1ccc(-c2nc(-c3ccc(-c4ccccn4)cc3)nc(-c3ccc(-c4cccc5c4-c4ccccc4C54c5ccccc5-c5ccccc54)cc3)n2)cc1]

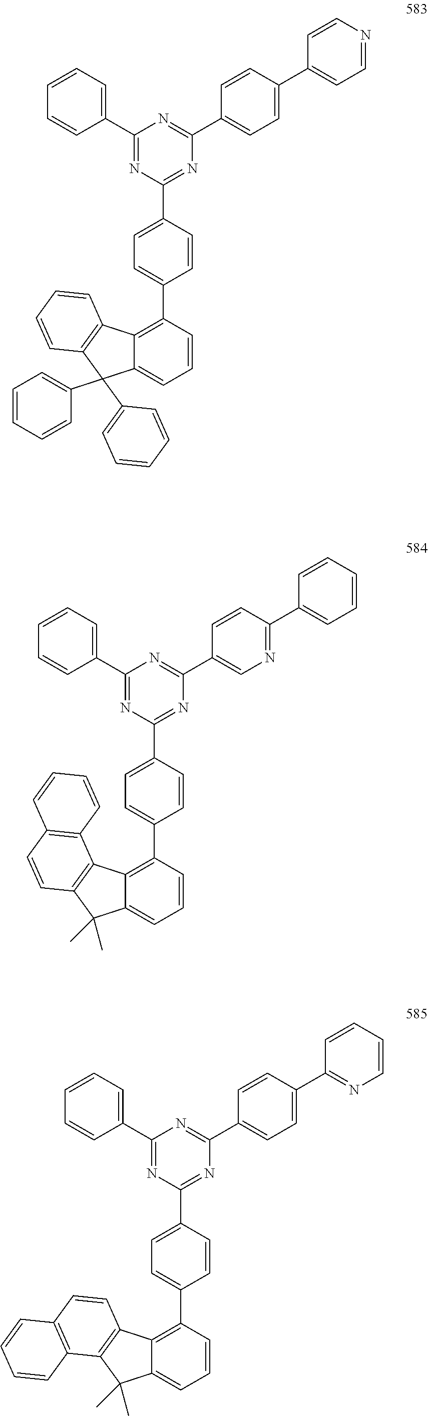 [SMILES: c1ccc(-c2nc(-c3ccc(-c4ccncc4)cc3)nc(-c3ccc(-c4cccc5c4-c4ccccc4C5(c4ccccc4)c4ccccc4)cc3)n2)cc1 ; CC1(C)c2cccc(-c3ccc(-c4nc(-c5ccccc5)nc(-c5ccc(-c6ccccc6)nc5)n4)cc3)c2-c2c1ccc1ccccc21 ; CC1(C)c2cccc(-c3ccc(-c4nc(-c5ccccc5)nc(-c5ccc(-c6ccccn6)cc5)n4)cc3)c2-c2ccc3ccccc3c21]

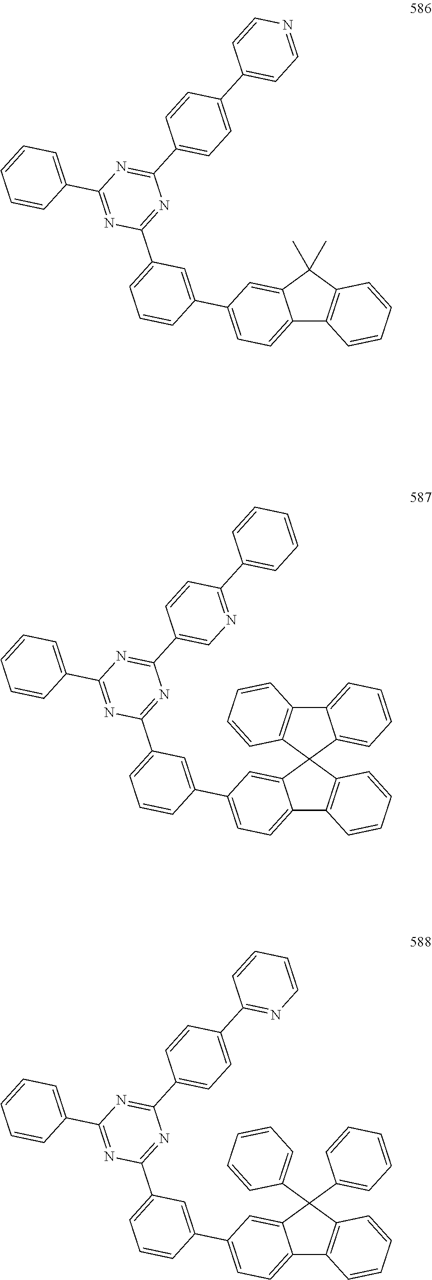 [SMILES: CC1(C)c2ccccc2-c2ccc(-c3cccc(-c4nc(-c5ccccc5)nc(-c5ccc(-c6ccncc6)cc5)n4)c3)cc21 ; c1ccc(-c2ccc(-c3nc(-c4ccccc4)nc(-c4cccc(-c5ccc6c(c5)C5(c7ccccc7-c7ccccc75)c5ccccc5-6)c4)n3)cn2)cc1 ; c1ccc(-c2nc(-c3ccc(-c4ccccn4)cc3)nc(-c3cccc(-c4ccc5c(c4)C(c4ccccc4)(c4ccccc4)c4ccccc4-5)c3)n2)cc1]

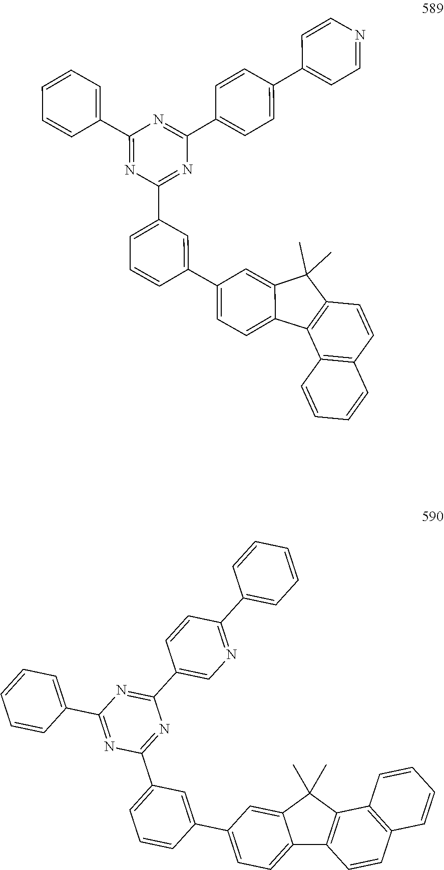 [SMILES: CC1(C)c2cc(-c3cccc(-c4nc(-c5ccccc5)nc(-c5ccc(-c6ccncc6)cc5)n4)c3)ccc2-c2c1ccc1ccccc21 ; CC1(C)c2cc(-c3cccc(-c4nc(-c5ccccc5)nc(-c5ccc(-c6ccccc6)nc5)n4)c3)ccc2-c2ccc3ccccc3c21]

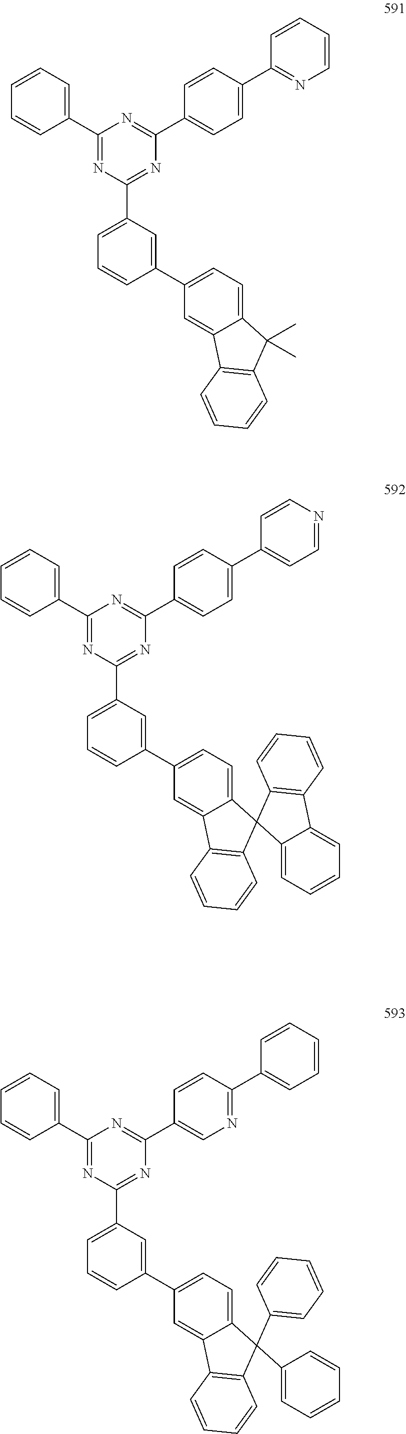 [SMILES: CC1(C)c2ccccc2-c2cc(-c3cccc(-c4nc(-c5ccccc5)nc(-c5ccc(-c6ccccn6)cc5)n4)c3)ccc21 ; c1ccc(-c2nc(-c3ccc(-c4ccncc4)cc3)nc(-c3cccc(-c4ccc5c(c4)-c4ccccc4C54c5ccccc5-c5ccccc54)c3)n2)cc1 ; c1ccc(-c2ccc(-c3nc(-c4ccccc4)nc(-c4cccc(-c5ccc6c(c5)-c5ccccc5C6(c5ccccc5)c5ccccc5)c4)n3)cn2)cc1]

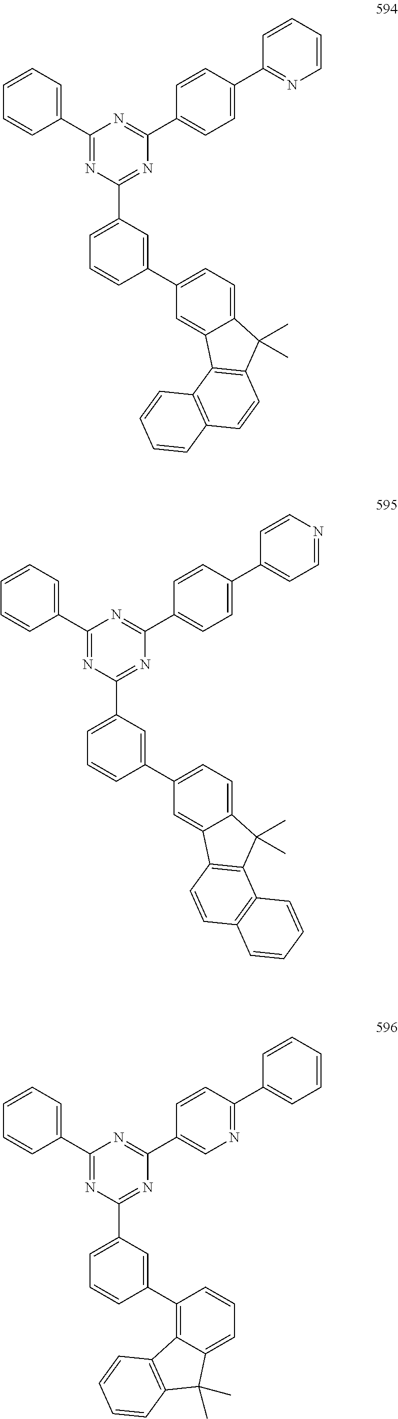 [SMILES: CC1(C)c2ccc(-c3cccc(-c4nc(-c5ccccc5)nc(-c5ccc(-c6ccccn6)cc5)n4)c3)cc2-c2c1ccc1ccccc21 ; CC1(C)c2ccc(-c3cccc(-c4nc(-c5ccccc5)nc(-c5ccc(-c6ccncc6)cc5)n4)c3)cc2-c2ccc3ccccc3c21 ; CC1(C)c2ccccc2-c2c(-c3cccc(-c4nc(-c5ccccc5)nc(-c5ccc(-c6ccccc6)nc5)n4)c3)cccc21]

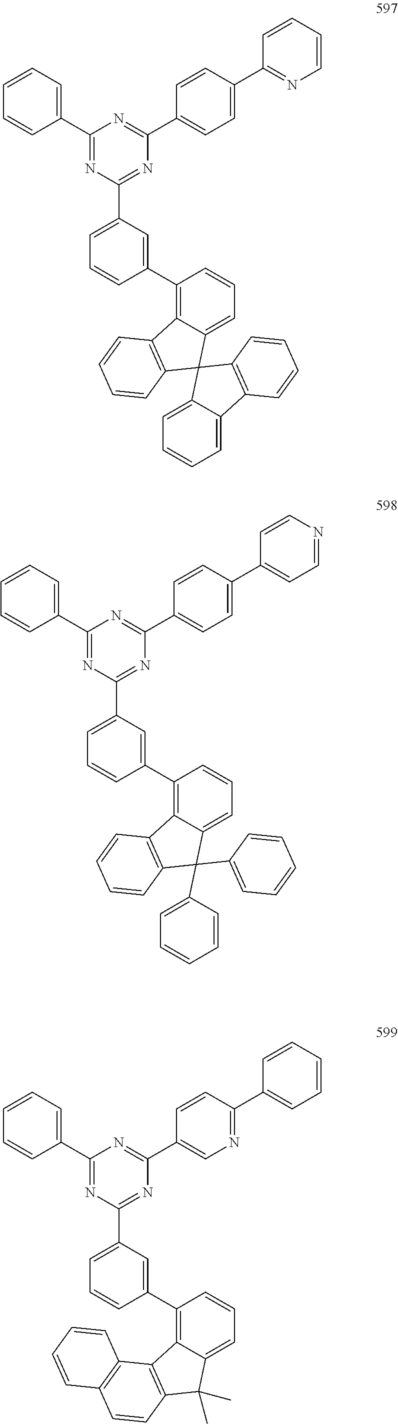 [SMILES: c1ccc(-c2nc(-c3ccc(-c4ccccn4)cc3)nc(-c3cccc(-c4cccc5c4-c4ccccc4C54c5ccccc5-c5ccccc54)c3)n2)cc1 ; c1ccc(-c2nc(-c3ccc(-c4ccncc4)cc3)nc(-c3cccc(-c4cccc5c4-c4ccccc4C5(c4ccccc4)c4ccccc4)c3)n2)cc1 ; CC1(C)c2cccc(-c3cccc(-c4nc(-c5ccccc5)nc(-c5ccc(-c6ccccc6)nc5)n4)c3)c2-c2c1ccc1ccccc21]

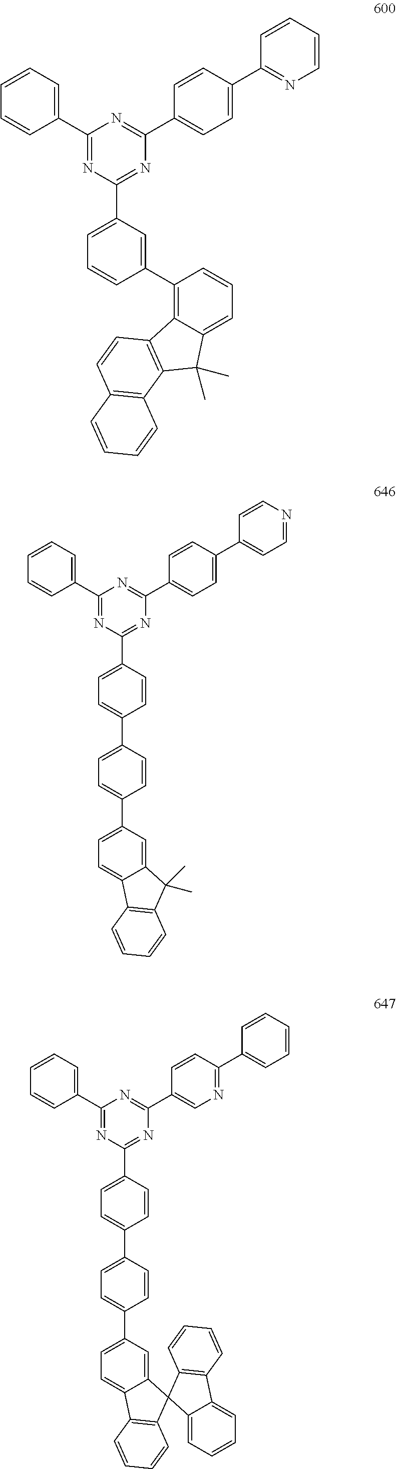 [SMILES: CC1(C)c2cccc(-c3cccc(-c4nc(-c5ccccc5)nc(-c5ccc(-c6ccccn6)cc5)n4)c3)c2-c2ccc3ccccc3c21 ; CC1(C)c2ccccc2-c2ccc(-c3ccc(-c4ccc(-c5nc(-c6ccccc6)nc(-c6ccc(-c7ccncc7)cc6)n5)cc4)cc3)cc21 ; c1ccc(-c2ccc(-c3nc(-c4ccccc4)nc(-c4ccc(-c5ccc(-c6ccc7c(c6)C6(c8ccccc8-c8ccccc86)c6ccccc6-7)cc5)cc4)n3)cn2)cc1]

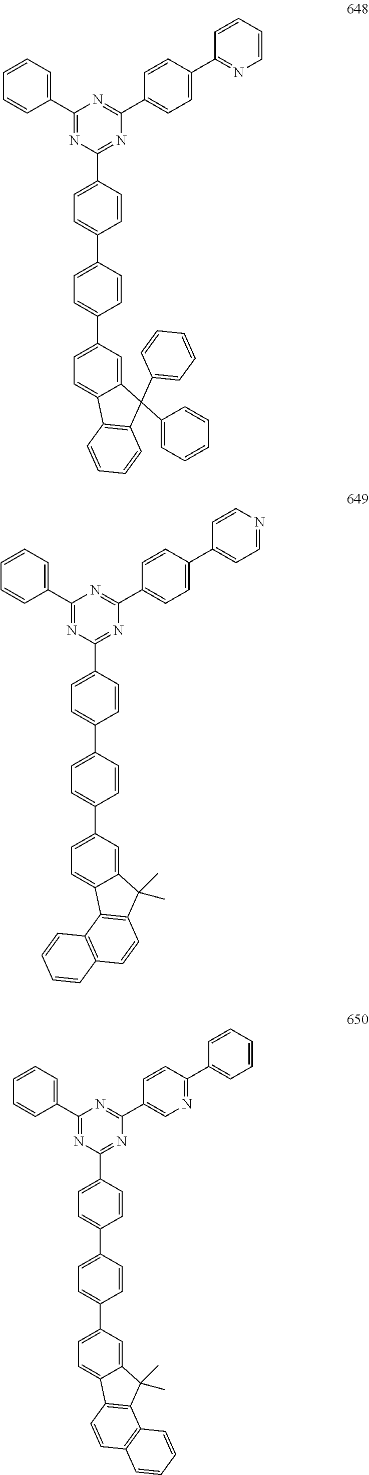 [SMILES: c1ccc(-c2nc(-c3ccc(-c4ccc(-c5ccc6c(c5)C(c5ccccc5)(c5ccccc5)c5ccccc5-6)cc4)cc3)nc(-c3ccc(-c4ccccn4)cc3)n2)cc1 ; CC1(C)c2cc(-c3ccc(-c4ccc(-c5nc(-c6ccccc6)nc(-c6ccc(-c7ccncc7)cc6)n5)cc4)cc3)ccc2-c2c1ccc1ccccc21 ; CC1(C)c2cc(-c3ccc(-c4ccc(-c5nc(-c6ccccc6)nc(-c6ccc(-c7ccccc7)nc6)n5)cc4)cc3)ccc2-c2ccc3ccccc3c21]

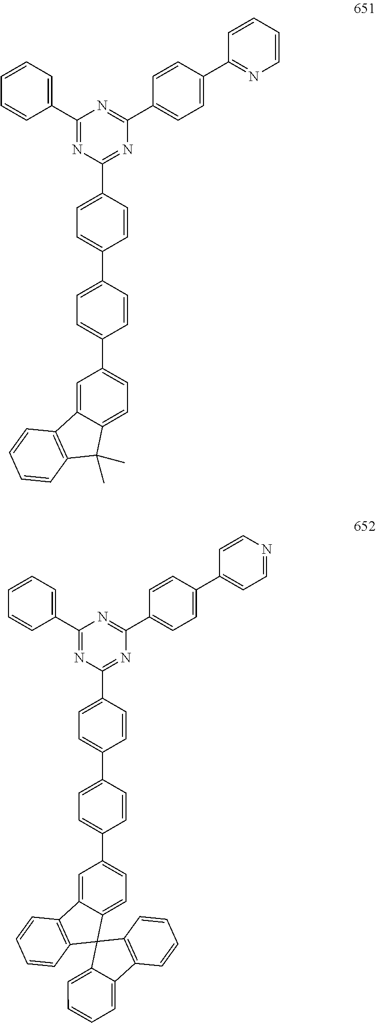 [SMILES: CC1(C)c2ccccc2-c2cc(-c3ccc(-c4ccc(-c5nc(-c6ccccc6)nc(-c6ccc(-c7ccccn7)cc6)n5)cc4)cc3)ccc21 ; c1ccc(-c2nc(-c3ccc(-c4ccncc4)cc3)nc(-c3ccc(-c4ccc(-c5ccc6c(c5)-c5ccccc5C65c6ccccc6-c6ccccc65)cc4)cc3)n2)cc1]

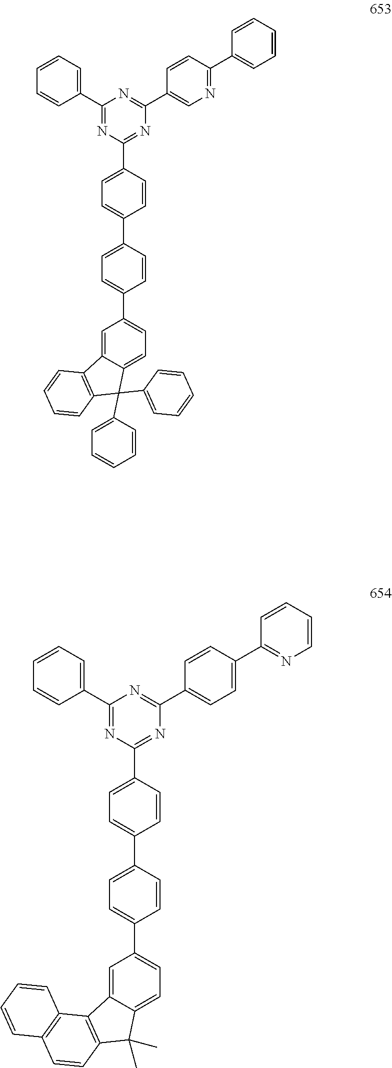 [SMILES: c1ccc(-c2ccc(-c3nc(-c4ccccc4)nc(-c4ccc(-c5ccc(-c6ccc7c(c6)-c6ccccc6C7(c6ccccc6)c6ccccc6)cc5)cc4)n3)cn2)cc1 ; CC1(C)c2ccc(-c3ccc(-c4ccc(-c5nc(-c6ccccc6)nc(-c6ccc(-c7ccccn7)cc6)n5)cc4)cc3)cc2-c2c1ccc1ccccc21]

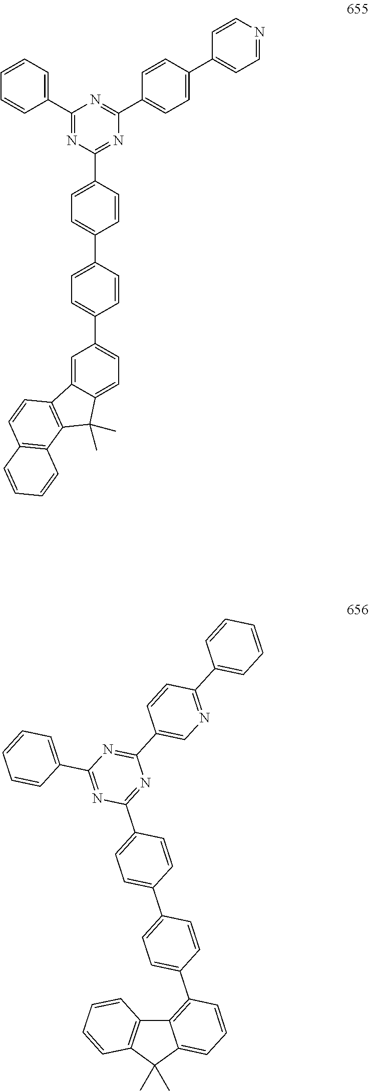 [SMILES: CC1(C)c2ccc(-c3ccc(-c4ccc(-c5nc(-c6ccccc6)nc(-c6ccc(-c7ccncc7)cc6)n5)cc4)cc3)cc2-c2ccc3ccccc3c21 ; CC1(C)c2ccccc2-c2c(-c3ccc(-c4ccc(-c5nc(-c6ccccc6)nc(-c6ccc(-c7ccccc7)nc6)n5)cc4)cc3)cccc21]

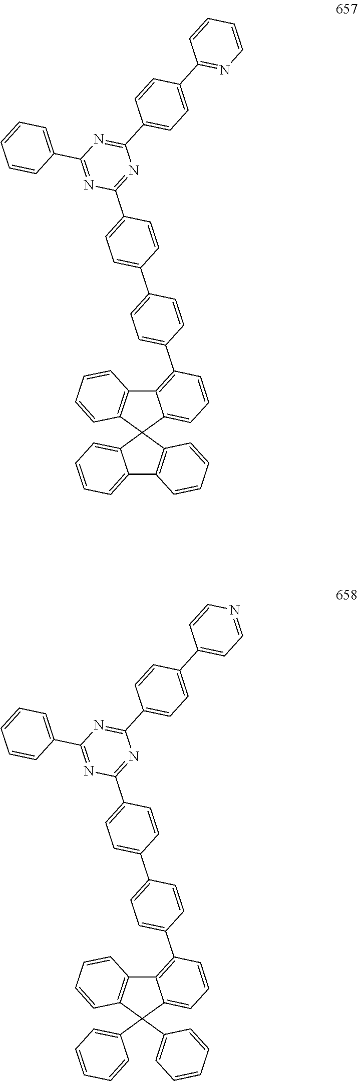 [SMILES: c1ccc(-c2nc(-c3ccc(-c4ccc(-c5cccc6c5-c5ccccc5C65c6ccccc6-c6ccccc65)cc4)cc3)nc(-c3ccc(-c4ccccn4)cc3)n2)cc1 ; c1ccc(-c2nc(-c3ccc(-c4ccncc4)cc3)nc(-c3ccc(-c4ccc(-c5cccc6c5-c5ccccc5C6(c5ccccc5)c5ccccc5)cc4)cc3)n2)cc1]

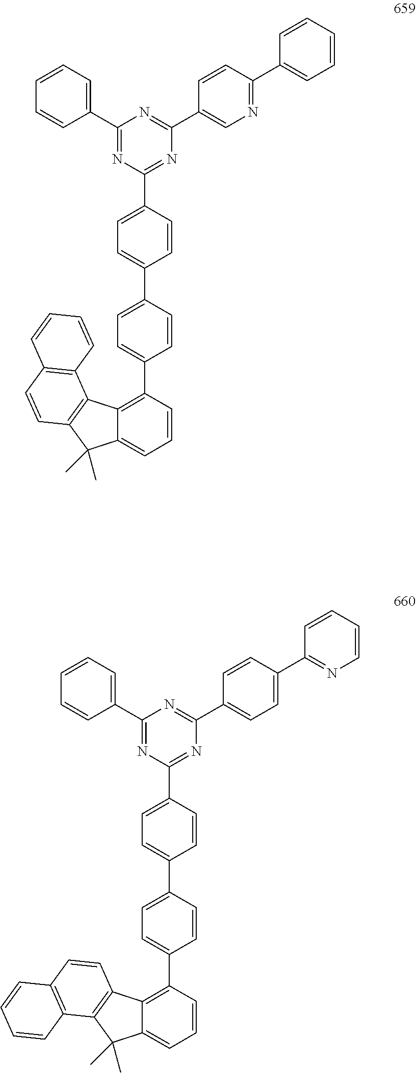 [SMILES: CC1(C)c2cccc(-c3ccc(-c4ccc(-c5nc(-c6ccccc6)nc(-c6ccc(-c7ccccc7)nc6)n5)cc4)cc3)c2-c2c1ccc1ccccc21 ; CC1(C)c2cccc(-c3ccc(-c4ccc(-c5nc(-c6ccccc6)nc(-c6ccc(-c7ccccn7)cc6)n5)cc4)cc3)c2-c2ccc3ccccc3c21]

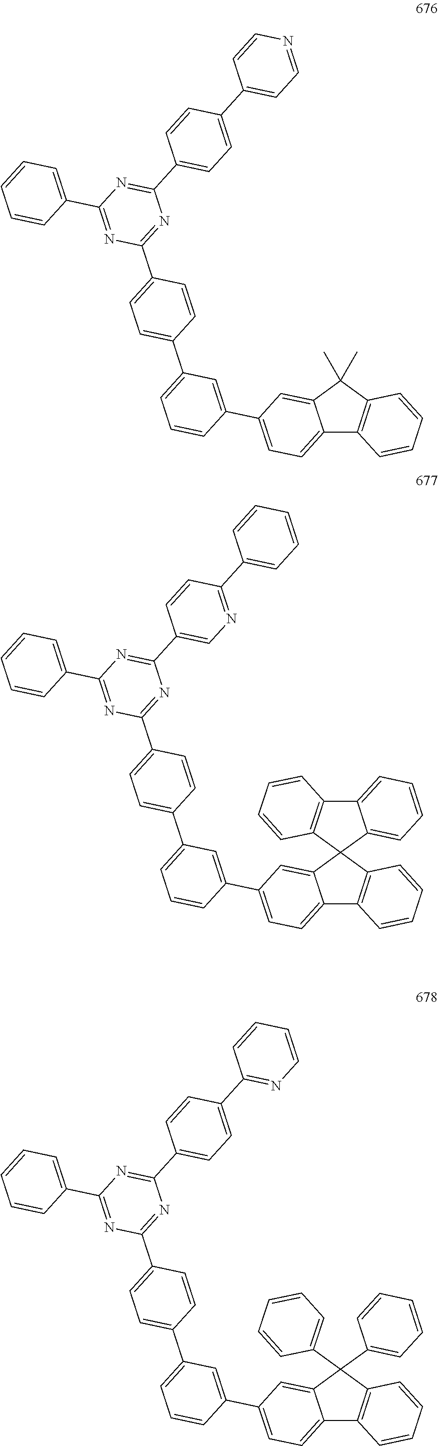 [SMILES: CC1(C)c2ccccc2-c2ccc(-c3cccc(-c4ccc(-c5nc(-c6ccccc6)nc(-c6ccc(-c7ccncc7)cc6)n5)cc4)c3)cc21 ; c1ccc(-c2ccc(-c3nc(-c4ccccc4)nc(-c4ccc(-c5cccc(-c6ccc7c(c6)C6(c8ccccc8-c8ccccc86)c6ccccc6-7)c5)cc4)n3)cn2)cc1 ; c1ccc(-c2nc(-c3ccc(-c4cccc(-c5ccc6c(c5)C(c5ccccc5)(c5ccccc5)c5ccccc5-6)c4)cc3)nc(-c3ccc(-c4ccccn4)cc3)n2)cc1]

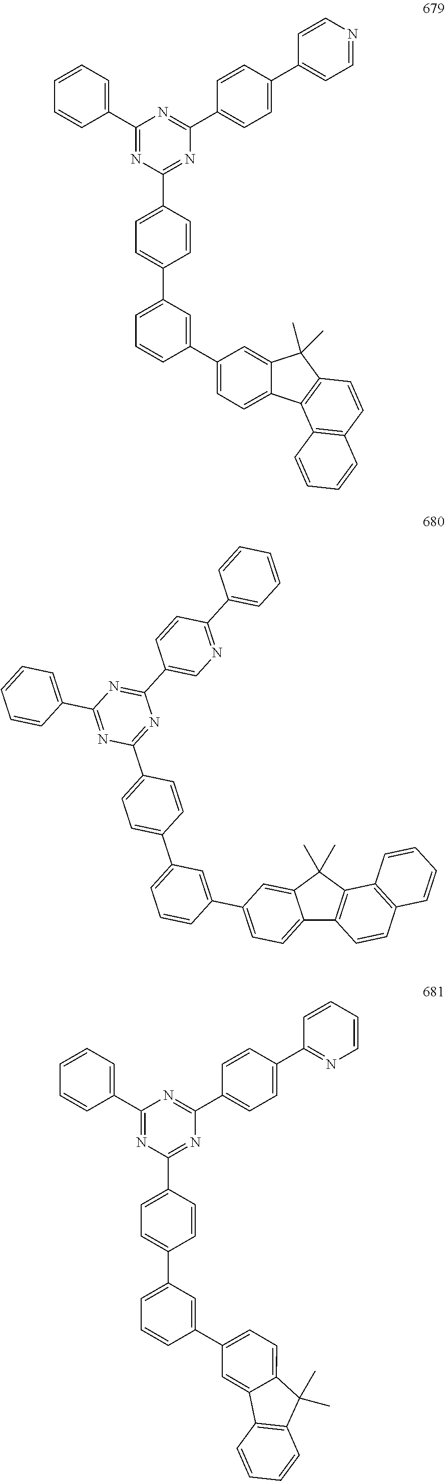 [SMILES: CC1(C)c2cc(-c3cccc(-c4ccc(-c5nc(-c6ccccc6)nc(-c6ccc(-c7ccncc7)cc6)n5)cc4)c3)ccc2-c2c1ccc1ccccc21 ; CC1(C)c2cc(-c3cccc(-c4ccc(-c5nc(-c6ccccc6)nc(-c6ccc(-c7ccccc7)nc6)n5)cc4)c3)ccc2-c2ccc3ccccc3c21 ; CC1(C)c2ccccc2-c2cc(-c3cccc(-c4ccc(-c5nc(-c6ccccc6)nc(-c6ccc(-c7ccccn7)cc6)n5)cc4)c3)ccc21]

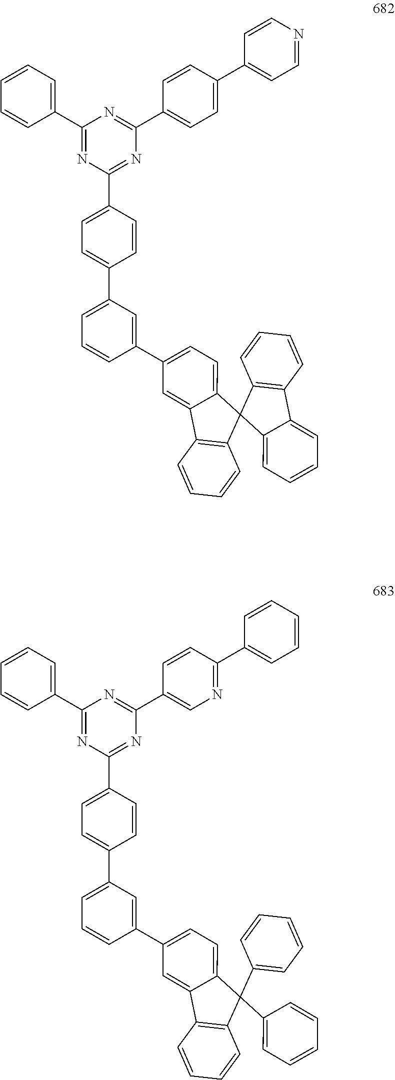 [SMILES: c1ccc(-c2nc(-c3ccc(-c4ccncc4)cc3)nc(-c3ccc(-c4cccc(-c5ccc6c(c5)-c5ccccc5C65c6ccccc6-c6ccccc65)c4)cc3)n2)cc1 ; c1ccc(-c2ccc(-c3nc(-c4ccccc4)nc(-c4ccc(-c5cccc(-c6ccc7c(c6)-c6ccccc6C7(c6ccccc6)c6ccccc6)c5)cc4)n3)cn2)cc1]

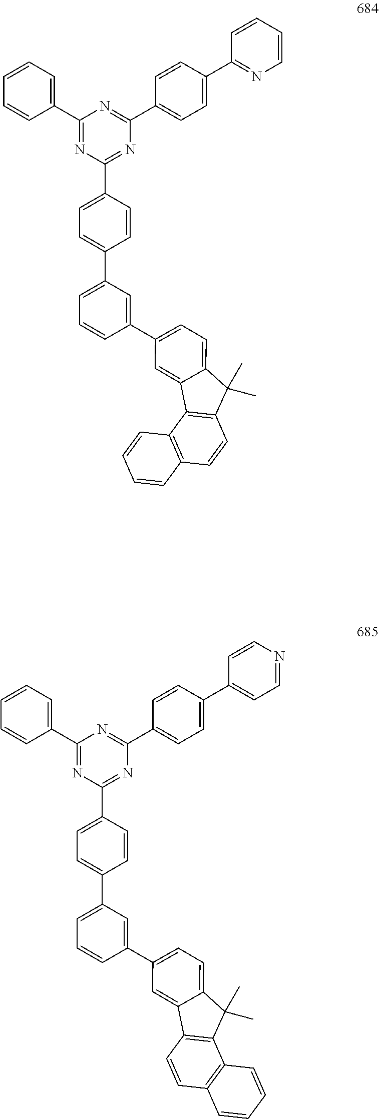 [SMILES: CC1(C)c2ccc(-c3cccc(-c4ccc(-c5nc(-c6ccccc6)nc(-c6ccc(-c7ccccn7)cc6)n5)cc4)c3)cc2-c2c1ccc1ccccc21 ; CC1(C)c2ccc(-c3cccc(-c4ccc(-c5nc(-c6ccccc6)nc(-c6ccc(-c7ccncc7)cc6)n5)cc4)c3)cc2-c2ccc3ccccc3c21]

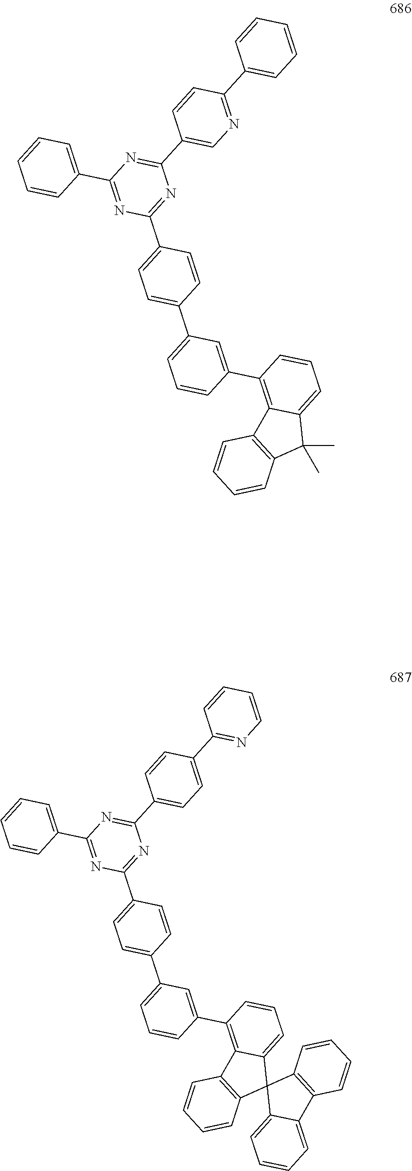 [SMILES: CC1(C)c2ccccc2-c2c(-c3cccc(-c4ccc(-c5nc(-c6ccccc6)nc(-c6ccc(-c7ccccc7)nc6)n5)cc4)c3)cccc21 ; c1ccc(-c2nc(-c3ccc(-c4cccc(-c5cccc6c5-c5ccccc5C65c6ccccc6-c6ccccc65)c4)cc3)nc(-c3ccc(-c4ccccn4)cc3)n2)cc1]

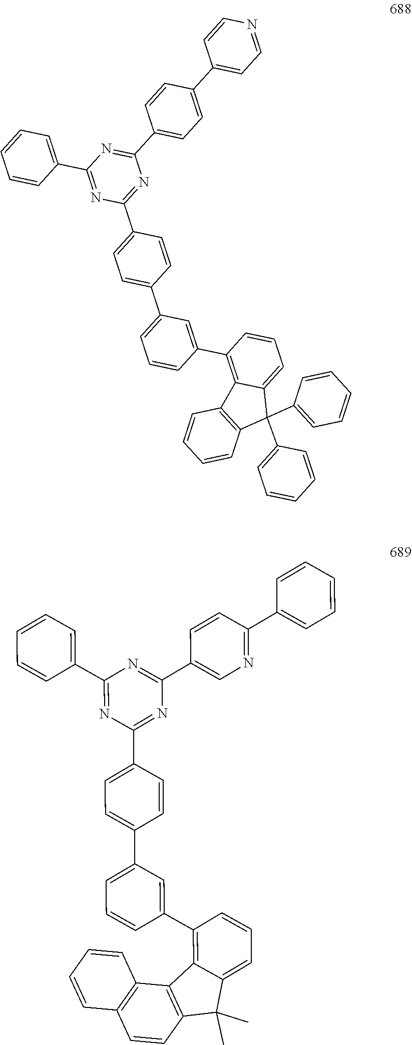 [SMILES: c1ccc(-c2nc(-c3ccc(-c4ccncc4)cc3)nc(-c3ccc(-c4cccc(-c5cccc6c5-c5ccccc5C6(c5ccccc5)c5ccccc5)c4)cc3)n2)cc1 ; CC1(C)c2cccc(-c3cccc(-c4ccc(-c5nc(-c6ccccc6)nc(-c6ccc(-c7ccccc7)nc6)n5)cc4)c3)c2-c2c1ccc1ccccc21]

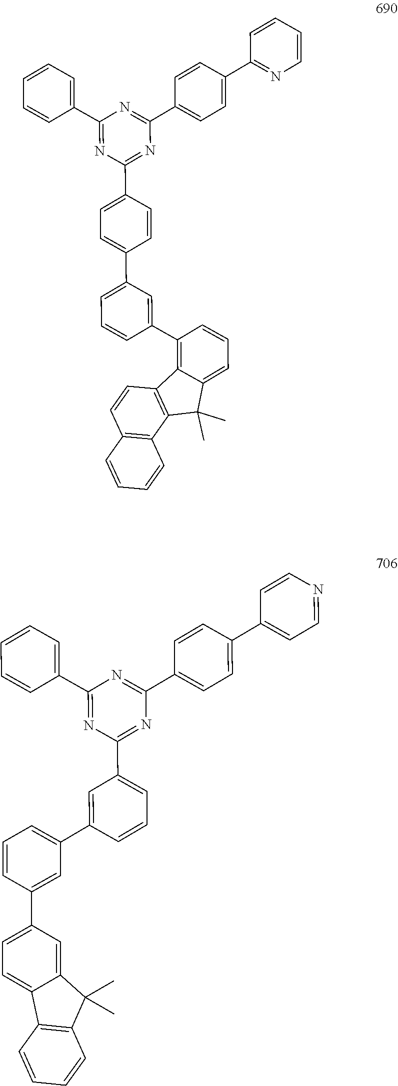 [SMILES: CC1(C)c2cccc(-c3cccc(-c4ccc(-c5nc(-c6ccccc6)nc(-c6ccc(-c7ccccn7)cc6)n5)cc4)c3)c2-c2ccc3ccccc3c21 ; CC1(C)c2ccccc2-c2ccc(-c3cccc(-c4cccc(-c5nc(-c6ccccc6)nc(-c6ccc(-c7ccncc7)cc6)n5)c4)c3)cc21]

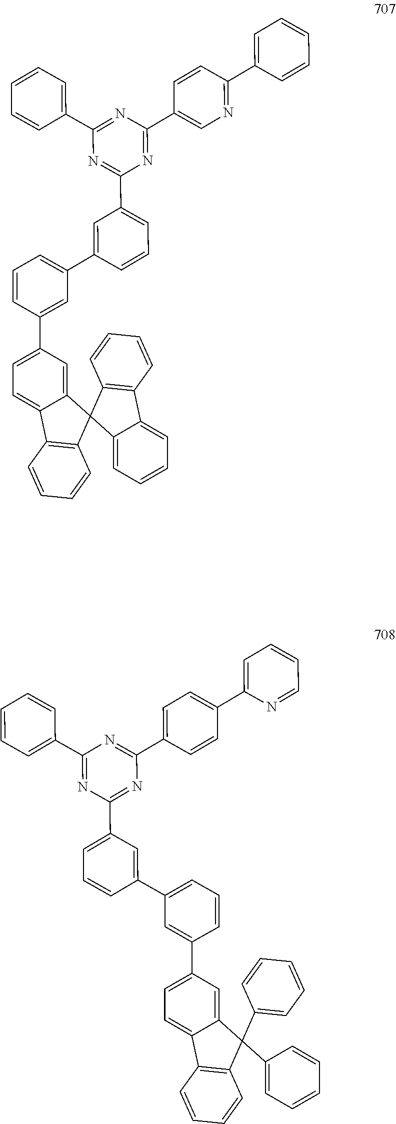 [SMILES: c1ccc(-c2ccc(-c3nc(-c4ccccc4)nc(-c4cccc(-c5cccc(-c6ccc7c(c6)C6(c8ccccc8-c8ccccc86)c6ccccc6-7)c5)c4)n3)cn2)cc1 ; c1ccc(-c2nc(-c3ccc(-c4ccccn4)cc3)nc(-c3cccc(-c4cccc(-c5ccc6c(c5)C(c5ccccc5)(c5ccccc5)c5ccccc5-6)c4)c3)n2)cc1]

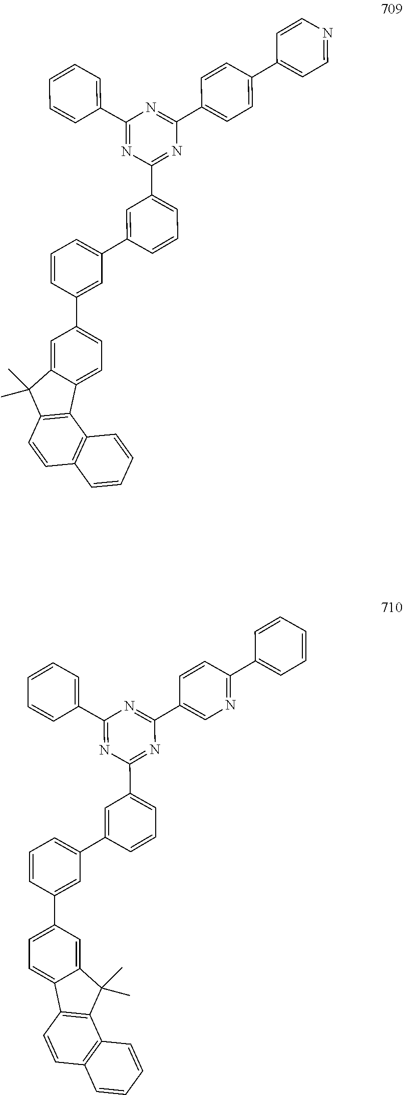 [SMILES: CC1(C)c2cc(-c3cccc(-c4cccc(-c5nc(-c6ccccc6)nc(-c6ccc(-c7ccncc7)cc6)n5)c4)c3)ccc2-c2c1ccc1ccccc21 ; CC1(C)c2cc(-c3cccc(-c4cccc(-c5nc(-c6ccccc6)nc(-c6ccc(-c7ccccc7)nc6)n5)c4)c3)ccc2-c2ccc3ccccc3c21]

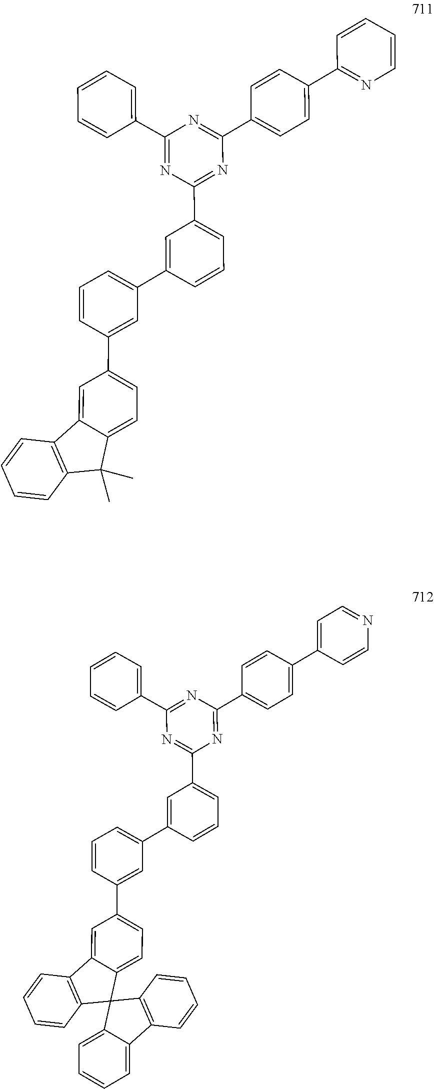 [SMILES: CC1(C)c2ccccc2-c2cc(-c3cccc(-c4cccc(-c5nc(-c6ccccc6)nc(-c6ccc(-c7ccccn7)cc6)n5)c4)c3)ccc21 ; c1ccc(-c2nc(-c3ccc(-c4ccncc4)cc3)nc(-c3cccc(-c4cccc(-c5ccc6c(c5)-c5ccccc5C65c6ccccc6-c6ccccc65)c4)c3)n2)cc1]

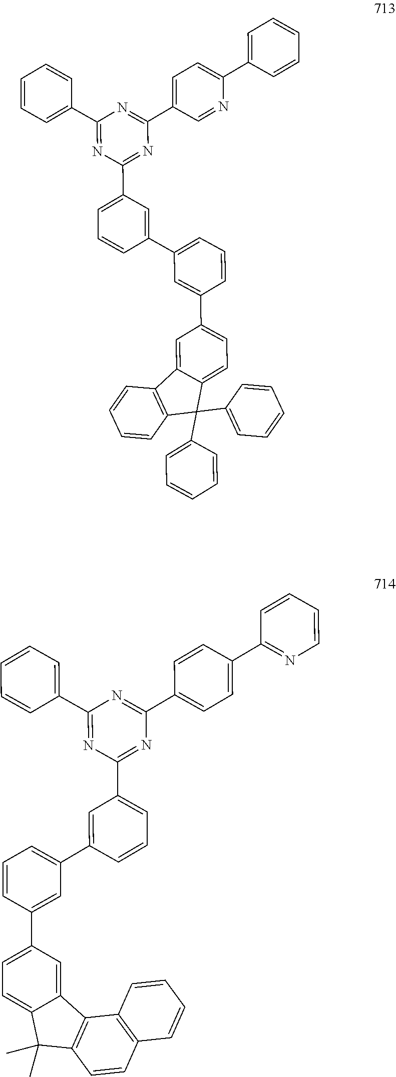 [SMILES: c1ccc(-c2ccc(-c3nc(-c4ccccc4)nc(-c4cccc(-c5cccc(-c6ccc7c(c6)-c6ccccc6C7(c6ccccc6)c6ccccc6)c5)c4)n3)cn2)cc1 ; CC1(C)c2ccc(-c3cccc(-c4cccc(-c5nc(-c6ccccc6)nc(-c6ccc(-c7ccccn7)cc6)n5)c4)c3)cc2-c2c1ccc1ccccc21]

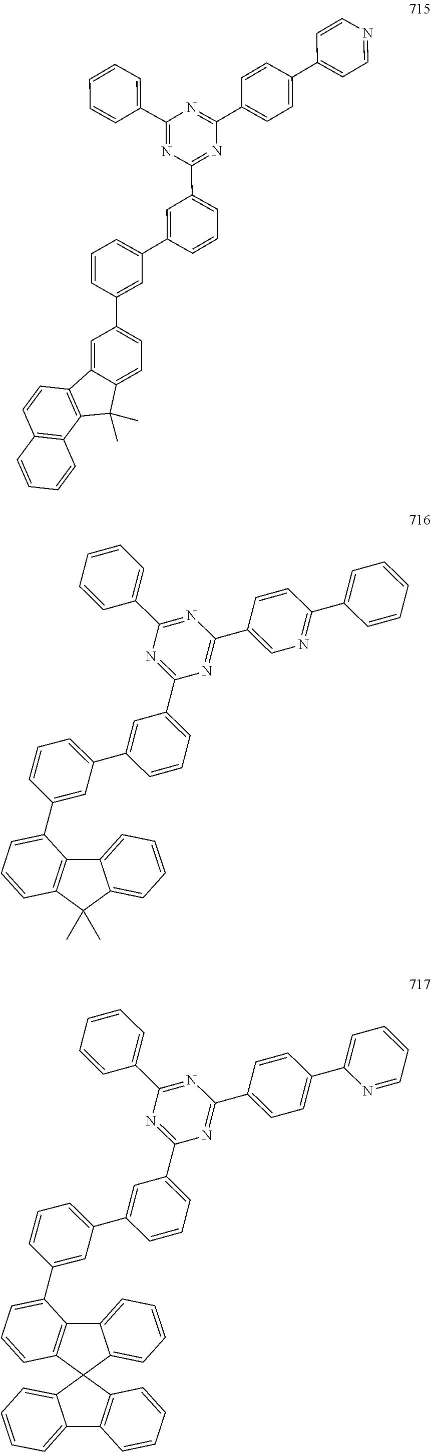 [SMILES: CC1(C)c2ccc(-c3cccc(-c4cccc(-c5nc(-c6ccccc6)nc(-c6ccc(-c7ccncc7)cc6)n5)c4)c3)cc2-c2ccc3ccccc3c21 ; CC1(C)c2ccccc2-c2c(-c3cccc(-c4cccc(-c5nc(-c6ccccc6)nc(-c6ccc(-c7ccccc7)nc6)n5)c4)c3)cccc21 ; c1ccc(-c2nc(-c3ccc(-c4ccccn4)cc3)nc(-c3cccc(-c4cccc(-c5cccc6c5-c5ccccc5C65c6ccccc6-c6ccccc65)c4)c3)n2)cc1]

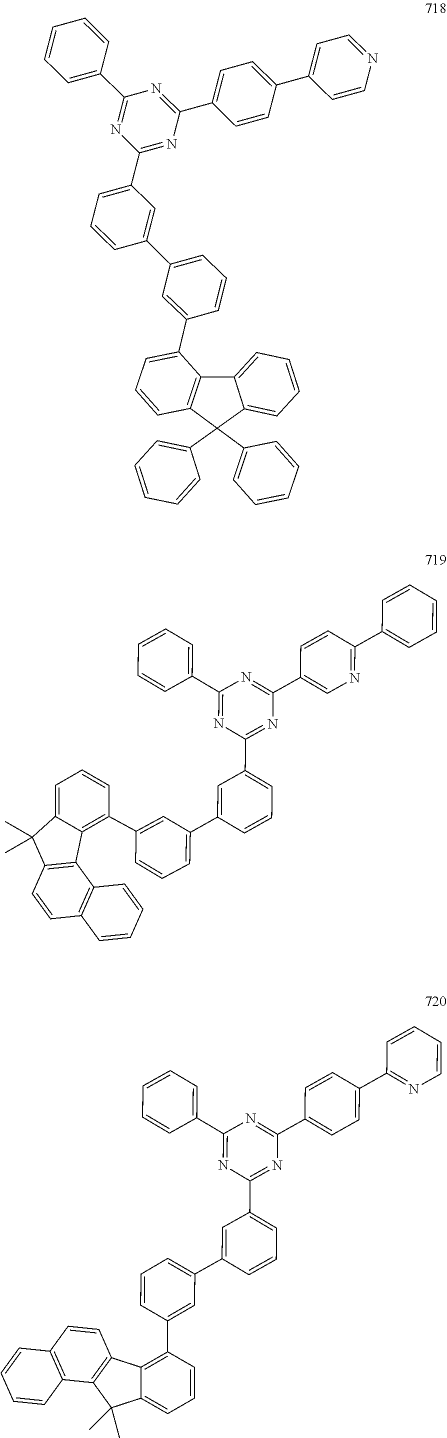 [SMILES: c1ccc(-c2nc(-c3ccc(-c4ccncc4)cc3)nc(-c3cccc(-c4cccc(-c5cccc6c5-c5ccccc5C6(c5ccccc5)c5ccccc5)c4)c3)n2)cc1 ; CC1(C)c2cccc(-c3cccc(-c4cccc(-c5nc(-c6ccccc6)nc(-c6ccc(-c7ccccc7)nc6)n5)c4)c3)c2-c2c1ccc1ccccc21 ; CC1(C)c2cccc(-c3cccc(-c4cccc(-c5nc(-c6ccccc6)nc(-c6ccc(-c7ccccn7)cc6)n5)c4)c3)c2-c2ccc3ccccc3c21]

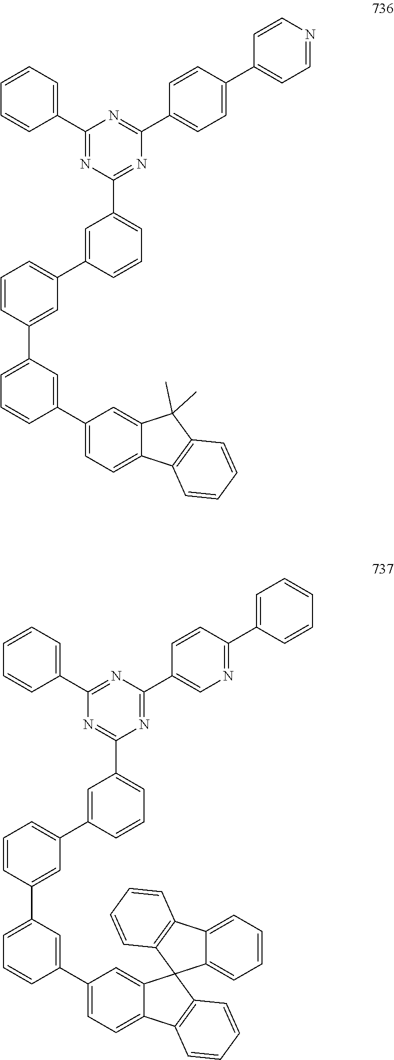 [SMILES: CC1(C)c2ccccc2-c2ccc(-c3cccc(-c4cccc(-c5cccc(-c6nc(-c7ccccc7)nc(-c7ccc(-c8ccncc8)cc7)n6)c5)c4)c3)cc21 ; c1ccc(-c2ccc(-c3nc(-c4ccccc4)nc(-c4cccc(-c5cccc(-c6cccc(-c7ccc8c(c7)C7(c9ccccc9-c9ccccc97)c7ccccc7-8)c6)c5)c4)n3)cn2)cc1]

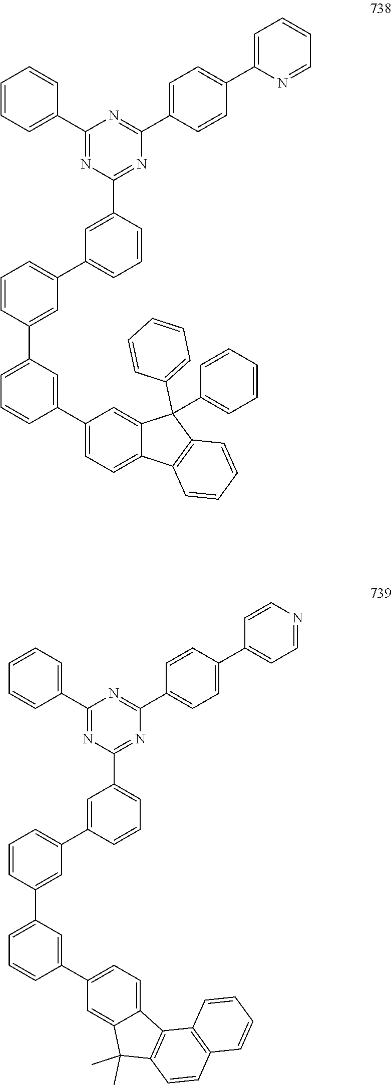

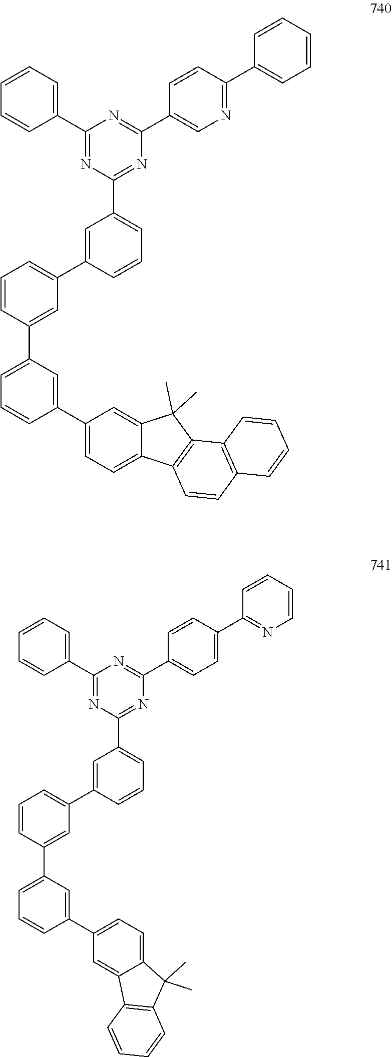 [SMILES: CC1(C)c2cc(-c3cccc(-c4cccc(-c5cccc(-c6nc(-c7ccccc7)nc(-c7ccc(-c8ccccc8)nc7)n6)c5)c4)c3)ccc2-c2ccc3ccccc3c21 ; CC1(C)c2ccccc2-c2cc(-c3cccc(-c4cccc(-c5cccc(-c6nc(-c7ccccc7)nc(-c7ccc(-c8ccccn8)cc7)n6)c5)c4)c3)ccc21]

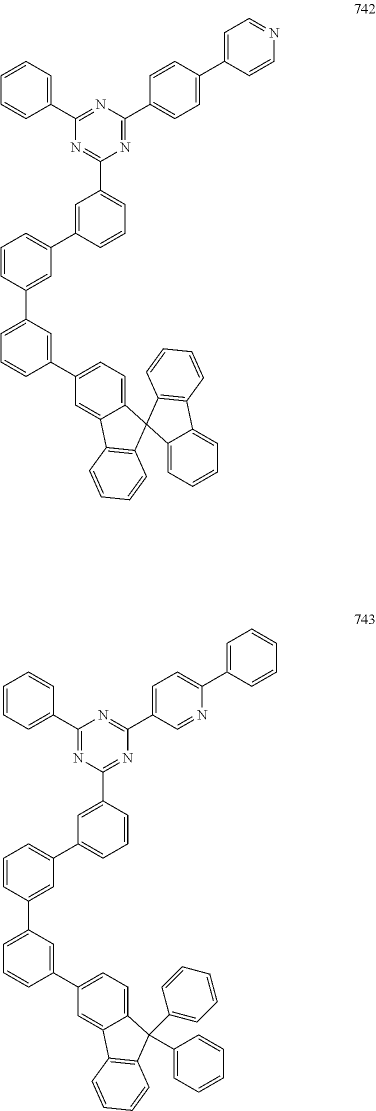 [SMILES: c1ccc(-c2nc(-c3ccc(-c4ccncc4)cc3)nc(-c3cccc(-c4cccc(-c5cccc(-c6ccc7c(c6)-c6ccccc6C76c7ccccc7-c7ccccc76)c5)c4)c3)n2)cc1 ; c1ccc(-c2ccc(-c3nc(-c4ccccc4)nc(-c4cccc(-c5cccc(-c6cccc(-c7ccc8c(c7)-c7ccccc7C8(c7ccccc7)c7ccccc7)c6)c5)c4)n3)cn2)cc1]

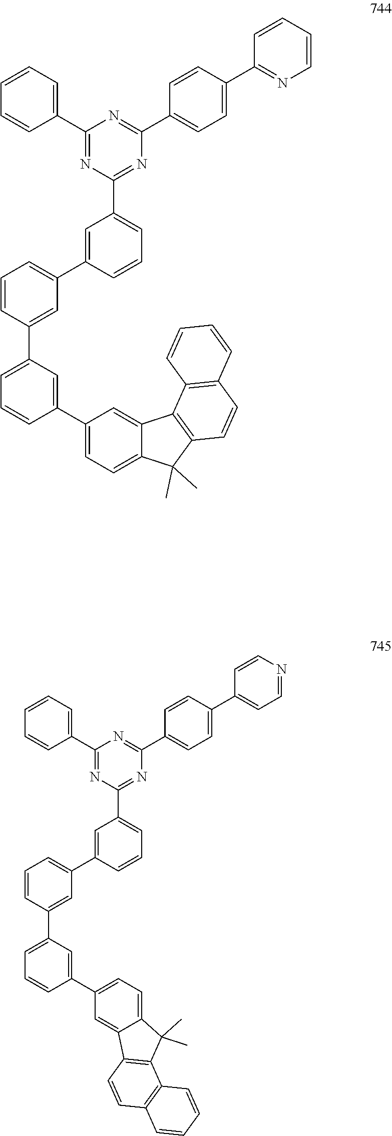 [SMILES: CC1(C)c2ccc(-c3cccc(-c4cccc(-c5cccc(-c6nc(-c7ccccc7)nc(-c7ccc(-c8ccccn8)cc7)n6)c5)c4)c3)cc2-c2c1ccc1ccccc21 ; CC1(C)c2ccc(-c3cccc(-c4cccc(-c5cccc(-c6nc(-c7ccccc7)nc(-c7ccc(-c8ccncc8)cc7)n6)c5)c4)c3)cc2-c2ccc3ccccc3c21]

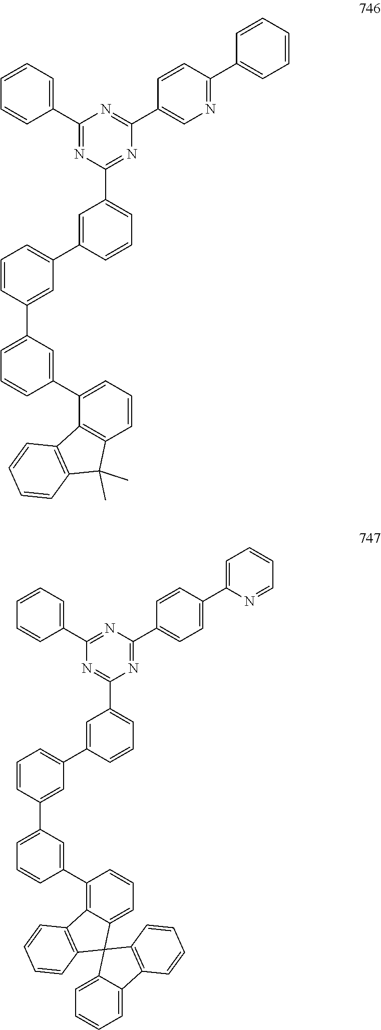 [SMILES: CC1(C)c2ccccc2-c2c(-c3cccc(-c4cccc(-c5cccc(-c6nc(-c7ccccc7)nc(-c7ccc(-c8ccccc8)nc7)n6)c5)c4)c3)cccc21 ; c1ccc(-c2nc(-c3ccc(-c4ccccn4)cc3)nc(-c3cccc(-c4cccc(-c5cccc(-c6cccc7c6-c6ccccc6C76c7ccccc7-c7ccccc76)c5)c4)c3)n2)cc1]

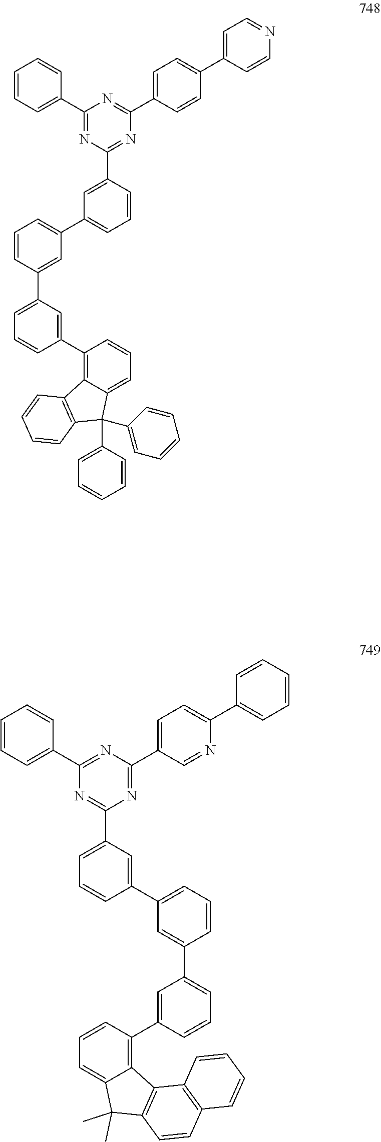 [SMILES: c1ccc(-c2nc(-c3ccc(-c4ccncc4)cc3)nc(-c3cccc(-c4cccc(-c5cccc(-c6cccc7c6-c6ccccc6C7(c6ccccc6)c6ccccc6)c5)c4)c3)n2)cc1 ; CC1(C)c2cccc(-c3cccc(-c4cccc(-c5cccc(-c6nc(-c7ccccc7)nc(-c7ccc(-c8ccccc8)nc7)n6)c5)c4)c3)c2-c2c1ccc1ccccc21]

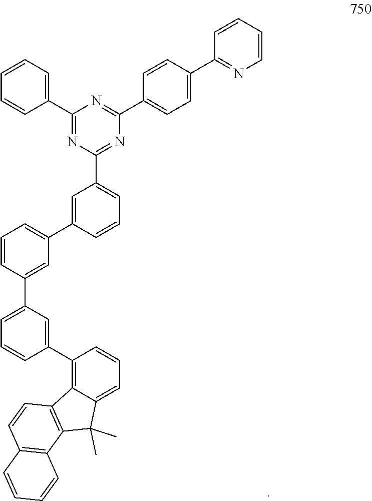 [SMILES: CC1(C)c2cccc(-c3cccc(-c4cccc(-c5cccc(-c6nc(-c7ccccc7)nc(-c7ccc(-c8ccccn8)cc7)n6)c5)c4)c3)c2-c2ccc3ccccc3c21]

9. An organic electroluminescent device, comprising an anode, a cathode and one or more organic layers disposed between the anode and the cathode,
wherein at least one of the one or more organic layers comprises the compound of the following Chemical Formula 1 according to claim 1: 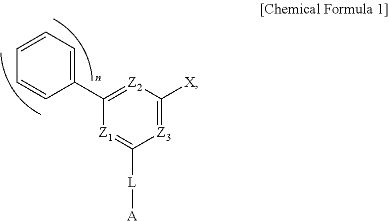
where in Chemical Formula 1,
Z₁ to Z₃ are each independently nitrogen, and
X is represented by the following Chemical Formula 2 or Chemical Formula 3, 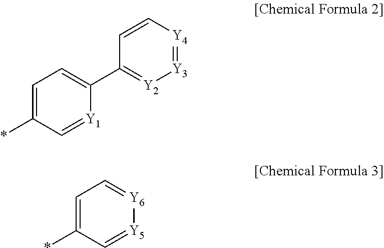
in Chemical Formula 2 and Chemical Formula 3,
one of Y₁ to Y₄ is nitrogen and the others are carbons, and one of Y₅ and Y₆ is nitrogen and the other is carbon,
* means a site where a bond with Chemical Formula 1 is made,
n is an integer ranging from 1 to 3,
L is a single bond, or selected from the group consisting of a C₆ to C₁₈ arylene group and a heteroarylene group having 5 to 18 nuclear atoms, and
A is represented by the following Chemical Formula 4, and 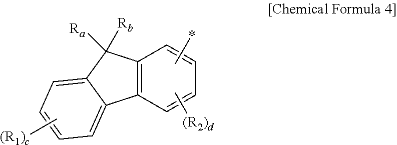
in Chemical Formula 4,
Rₐ and R_{b} are the same as or different from each other, each independently a C₁ to C₄₀ alkyl group or C₆ to C₆₀ aryl group, or bound with each other to form a fused ring,
R₁ and R₂ are the same as or different from each other, each independently selected from the group consisting of hydrogen, deuterium, a halogen group, a cyano group, a nitro group, an amino group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkyl boron group, a C₆ to C₆₀ aryl boron group, a C₁ to C₄₀ phosphine group, a C₁ to C₄₀ phosphine oxide group, and a C₆ to C₆₀ arylamine group, or bound with an adjacent group to form a fused ring,
c is an integer ranging from 0 to 4,
d is an integer ranging from 0 to 3,
* means a site where a bond with Chemical Formula 1 is made,
the alkyl group and the aryl group of Rₐ and R_{b}, the alkyl group, the alkenyl group, the alkynyl group, the cycloalkyl group, the heterocycloalkyl group, the aryl group, the heteroaryl group, the alkyloxy group, the aryloxy group, the alkylsilyl group, the arylsilyl group, the alkylboron group, the arylboron group, the phosphine group, the phosphine oxide group, and the arylamine group of R₁ and R₂, and the arylene group and the heteroarylene group of L are each independently substituted or unsubstituted with one or more kinds of substituents selected from the group consisting of deuterium, a halogen group, a cyano group, a nitro group, an amino group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkyl boron group, a C₆ to C₆₀ arylboron group, a C₁ to C₄₀ phosphine group, a C₁ to C₄₀ phosphine oxide group, and a C₆ to C₆₀ arylamine group, and when the substituents are plural in number, the plurality of substituents are the same as or different from each other.

10. The organic electroluminescent device of claim 9, wherein the compound represented by Chemical Formula 1 is a compound represented by any one of the following Chemical Formula 7 and Chemical Formula 10: 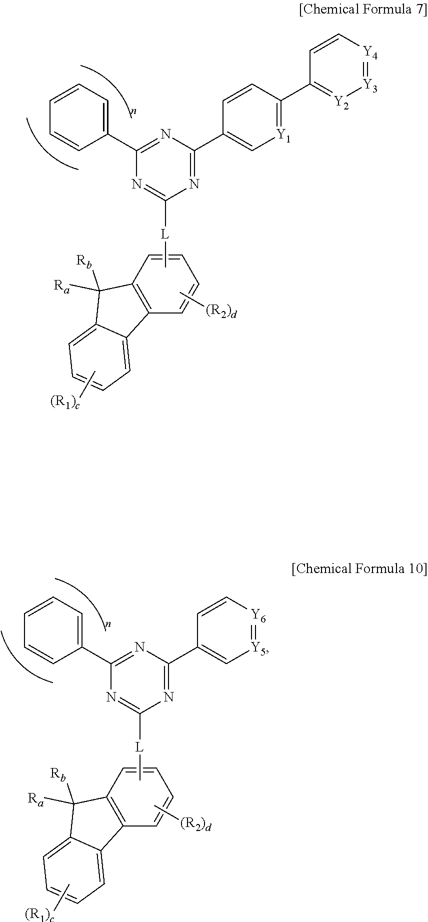
where in Chemical Formula 7 and Chemical Formula 10,
Rₐ, R_{b}, R₁, R₂, Y₁ to Y₆, L, c, d and n are the same as those defined in claim 9, respectively.

11. The organic electroluminescent device of claim 9, wherein in Chemical Formula 1, X is selected from the group consisting of the following structures represented by X-1 to X-6:

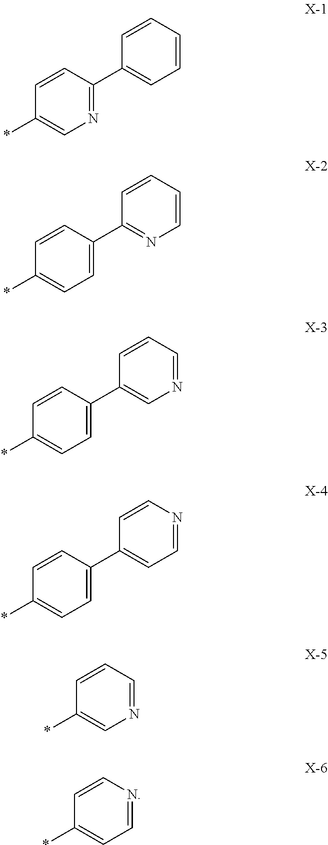 [SMILES: *c1ccc(-c2ccccc2)nc1 ; *c1ccc(-c2ccccn2)cc1 ; *c1ccc(-c2cccnc2)cc1 ; *c1ccc(-c2ccncc2)cc1 ; *c1cccnc1 ; *c1ccncc1]

12. The organic electroluminescent device of claim 9, wherein in Chemical Formula 1, a structure represented by 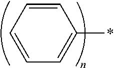
(* being a site where a bond is made) is selected from the group consisting of the following structures represented by Ar-1 to Ar-3:

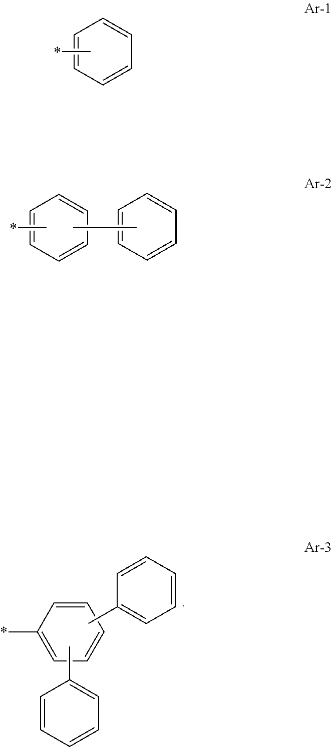

13. The organic electroluminescent device of claim 9, wherein Rₐ and R_{b} are each independently a methyl group or a phenyl group, or combined with each other to form a fused ring represented by 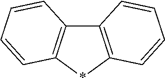 [SMILES: *1c2ccccc2c2ccccc12]
(* being a site where a bond is made).

14. The organic electroluminescent device of claim 9, wherein in Chemical Formula 1, A is selected from the group consisting of the following structures represented by A-1 to A-6:

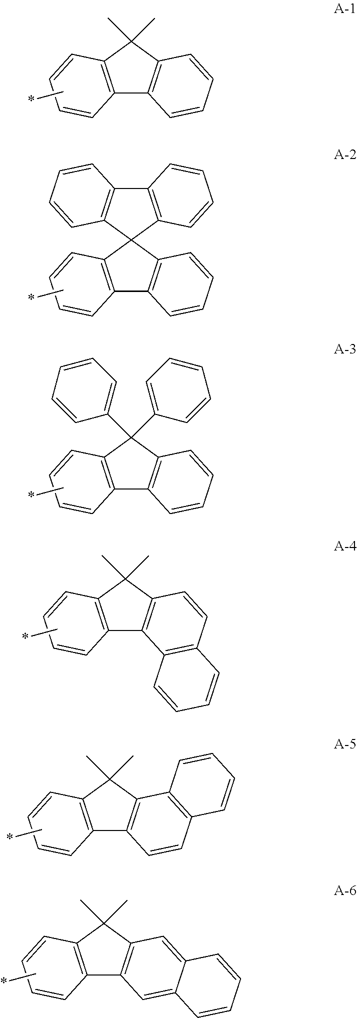

15. The organic electroluminescent device of claim 9, wherein in Chemical Formula 1, L is a single bond or a linking group selected from the following structures represented by L-1 to L-7:

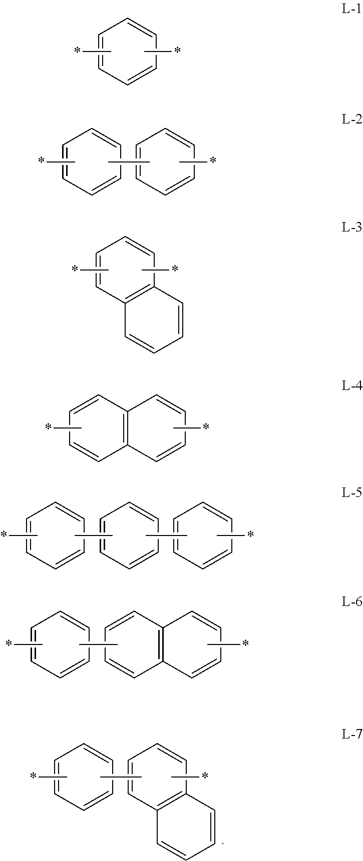

16. The organic electroluminescent device of claim 9, wherein the compound represented by Chemical Formula 1 is selected from the group consisting of the following compounds represented by 31-45, 61-90, 136-165, 226-255, 286-315, 331-345, 361-375, 391-405, 421-435, 466-480, 496-525, 571-600, 646-660, 676-690, 706-720, and 736-750:

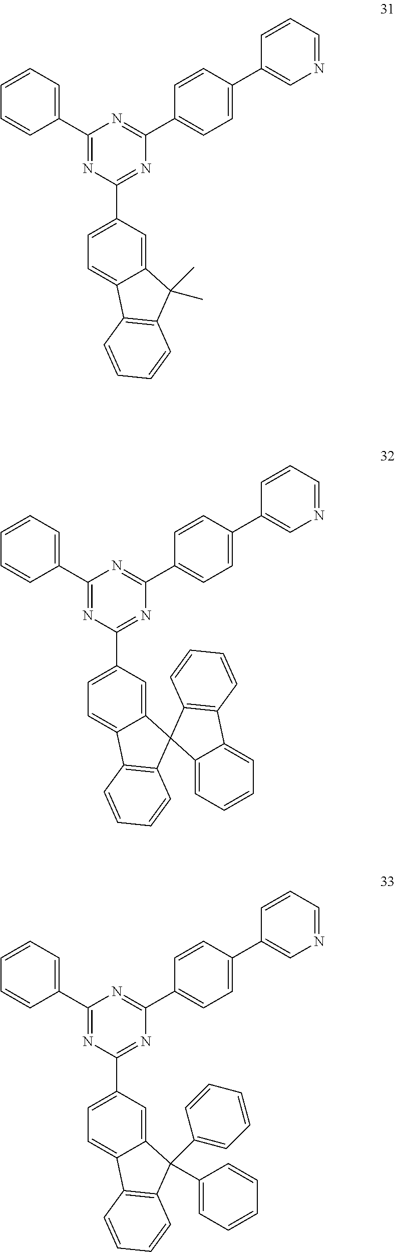 [SMILES: CC1(C)c2ccccc2-c2ccc(-c3nc(-c4ccccc4)nc(-c4ccc(-c5cccnc5)cc4)n3)cc21 ; c1ccc(-c2nc(-c3ccc(-c4cccnc4)cc3)nc(-c3ccc4c(c3)C3(c5ccccc5-c5ccccc53)c3ccccc3-4)n2)cc1 ; c1ccc(-c2nc(-c3ccc(-c4cccnc4)cc3)nc(-c3ccc4c(c3)C(c3ccccc3)(c3ccccc3)c3ccccc3-4)n2)cc1]

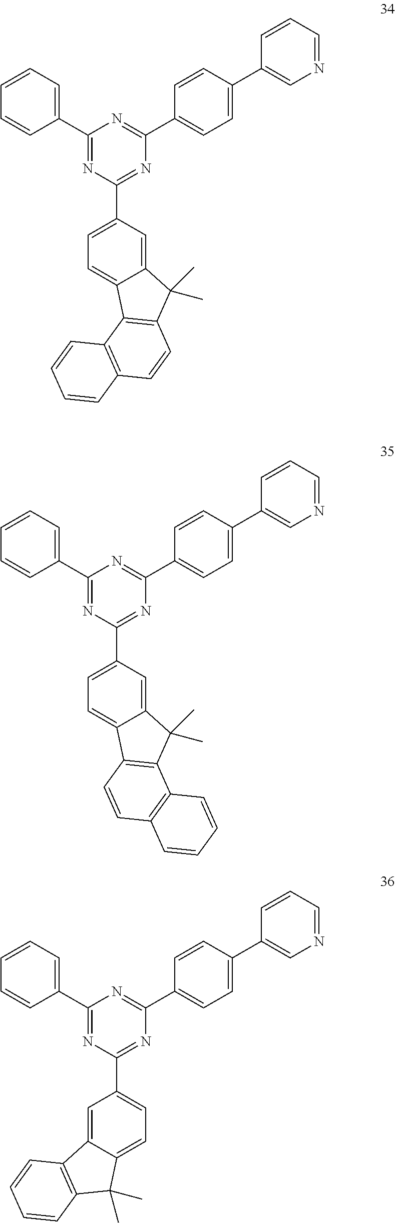 [SMILES: CC1(C)c2cc(-c3nc(-c4ccccc4)nc(-c4ccc(-c5cccnc5)cc4)n3)ccc2-c2c1ccc1ccccc21 ; CC1(C)c2cc(-c3nc(-c4ccccc4)nc(-c4ccc(-c5cccnc5)cc4)n3)ccc2-c2ccc3ccccc3c21 ; CC1(C)c2ccccc2-c2cc(-c3nc(-c4ccccc4)nc(-c4ccc(-c5cccnc5)cc4)n3)ccc21]

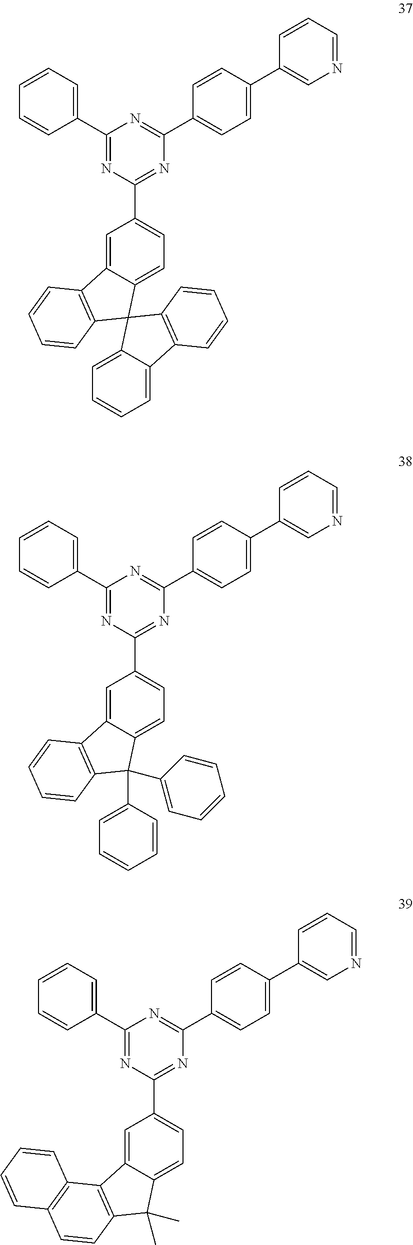 [SMILES: c1ccc(-c2nc(-c3ccc(-c4cccnc4)cc3)nc(-c3ccc4c(c3)-c3ccccc3C43c4ccccc4-c4ccccc43)n2)cc1 ; c1ccc(-c2nc(-c3ccc(-c4cccnc4)cc3)nc(-c3ccc4c(c3)-c3ccccc3C4(c3ccccc3)c3ccccc3)n2)cc1 ; CC1(C)c2ccc(-c3nc(-c4ccccc4)nc(-c4ccc(-c5cccnc5)cc4)n3)cc2-c2c1ccc1ccccc21]

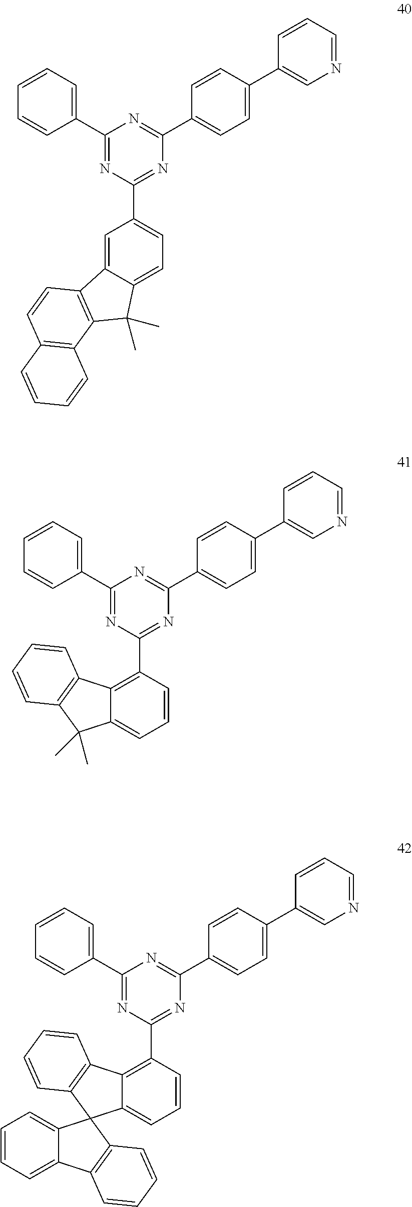 [SMILES: CC1(C)c2ccc(-c3nc(-c4ccccc4)nc(-c4ccc(-c5cccnc5)cc4)n3)cc2-c2ccc3ccccc3c21 ; CC1(C)c2ccccc2-c2c(-c3nc(-c4ccccc4)nc(-c4ccc(-c5cccnc5)cc4)n3)cccc21 ; c1ccc(-c2nc(-c3ccc(-c4cccnc4)cc3)nc(-c3cccc4c3-c3ccccc3C43c4ccccc4-c4ccccc43)n2)cc1]

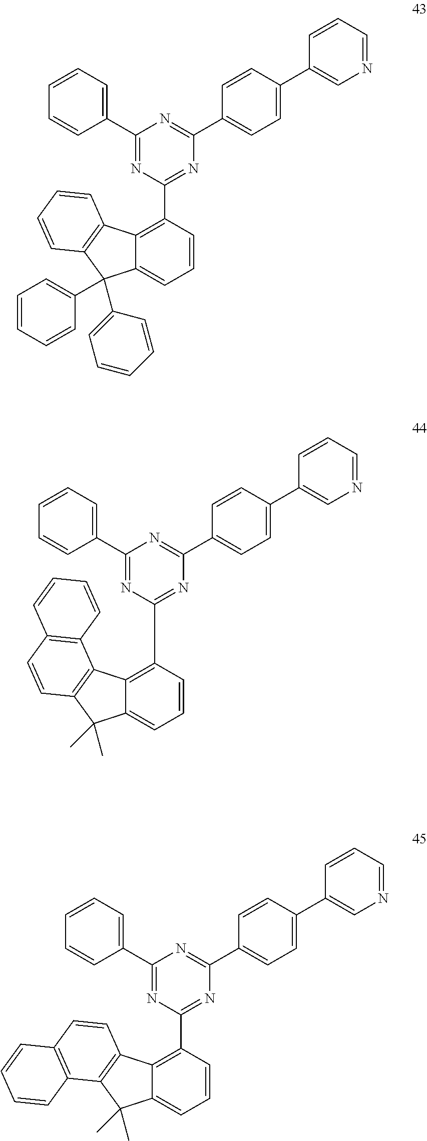 [SMILES: c1ccc(-c2nc(-c3ccc(-c4cccnc4)cc3)nc(-c3cccc4c3-c3ccccc3C4(c3ccccc3)c3ccccc3)n2)cc1 ; CC1(C)c2cccc(-c3nc(-c4ccccc4)nc(-c4ccc(-c5cccnc5)cc4)n3)c2-c2c1ccc1ccccc21 ; CC1(C)c2cccc(-c3nc(-c4ccccc4)nc(-c4ccc(-c5cccnc5)cc4)n3)c2-c2ccc3ccccc3c21]

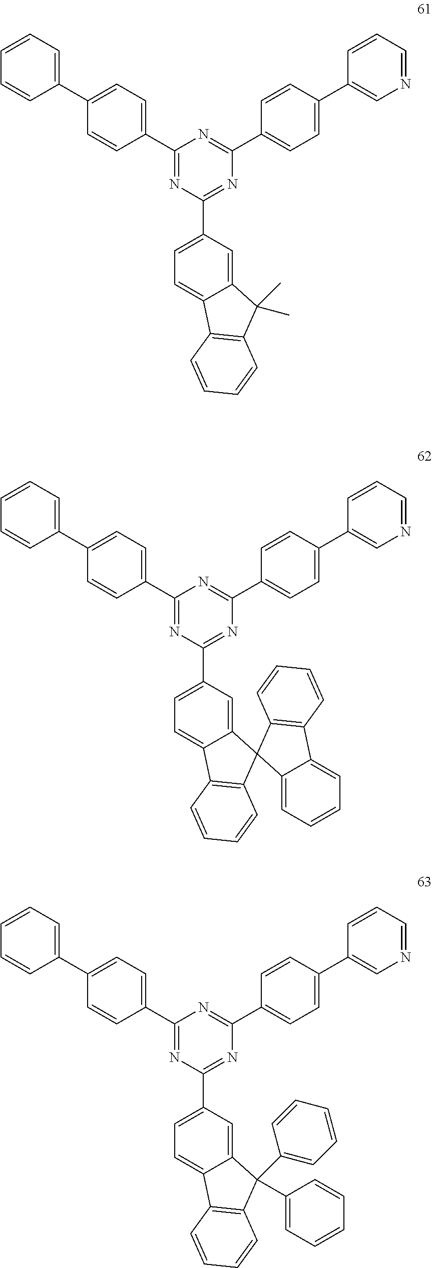 [SMILES: CC1(C)c2ccccc2-c2ccc(-c3nc(-c4ccc(-c5ccccc5)cc4)nc(-c4ccc(-c5cccnc5)cc4)n3)cc21 ; c1ccc(-c2ccc(-c3nc(-c4ccc(-c5cccnc5)cc4)nc(-c4ccc5c(c4)C4(c6ccccc6-c6ccccc64)c4ccccc4-5)n3)cc2)cc1 ; c1ccc(-c2ccc(-c3nc(-c4ccc(-c5cccnc5)cc4)nc(-c4ccc5c(c4)C(c4ccccc4)(c4ccccc4)c4ccccc4-5)n3)cc2)cc1]

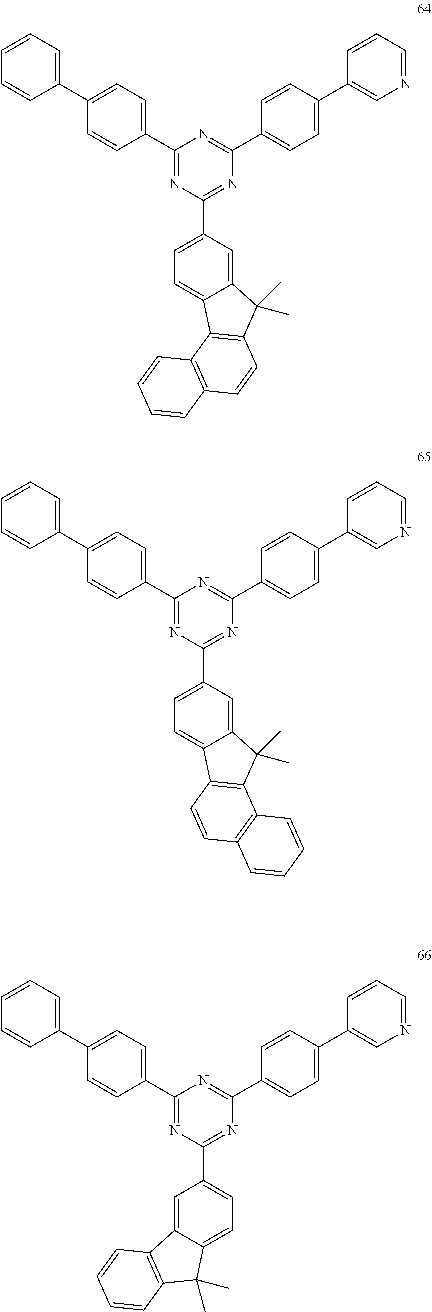 [SMILES: CC1(C)c2cc(-c3nc(-c4ccc(-c5ccccc5)cc4)nc(-c4ccc(-c5cccnc5)cc4)n3)ccc2-c2c1ccc1ccccc21 ; CC1(C)c2cc(-c3nc(-c4ccc(-c5ccccc5)cc4)nc(-c4ccc(-c5cccnc5)cc4)n3)ccc2-c2ccc3ccccc3c21 ; CC1(C)c2ccccc2-c2cc(-c3nc(-c4ccc(-c5ccccc5)cc4)nc(-c4ccc(-c5cccnc5)cc4)n3)ccc21]

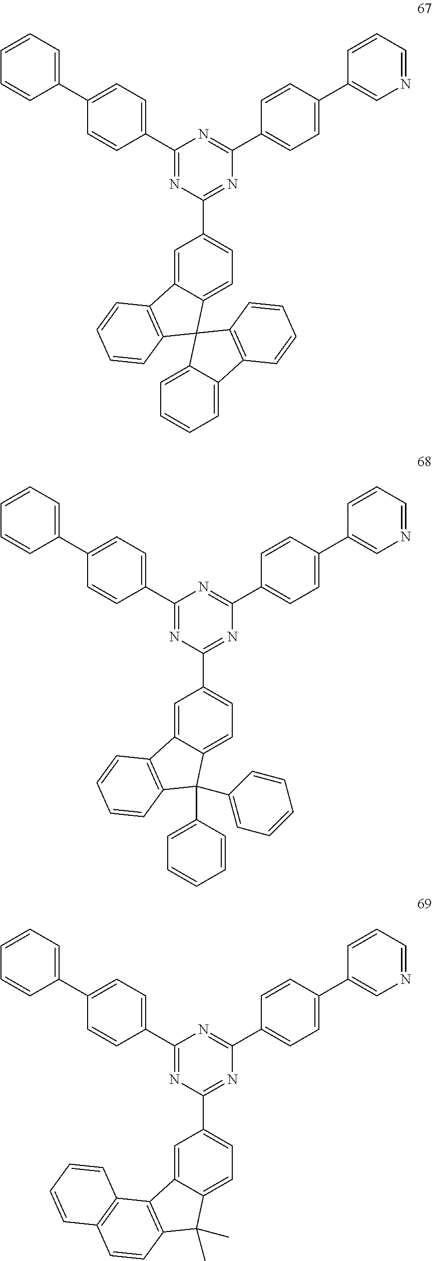 [SMILES: c1ccc(-c2ccc(-c3nc(-c4ccc(-c5cccnc5)cc4)nc(-c4ccc5c(c4)-c4ccccc4C54c5ccccc5-c5ccccc54)n3)cc2)cc1 ; c1ccc(-c2ccc(-c3nc(-c4ccc(-c5cccnc5)cc4)nc(-c4ccc5c(c4)-c4ccccc4C5(c4ccccc4)c4ccccc4)n3)cc2)cc1 ; CC1(C)c2ccc(-c3nc(-c4ccc(-c5ccccc5)cc4)nc(-c4ccc(-c5cccnc5)cc4)n3)cc2-c2c1ccc1ccccc21]

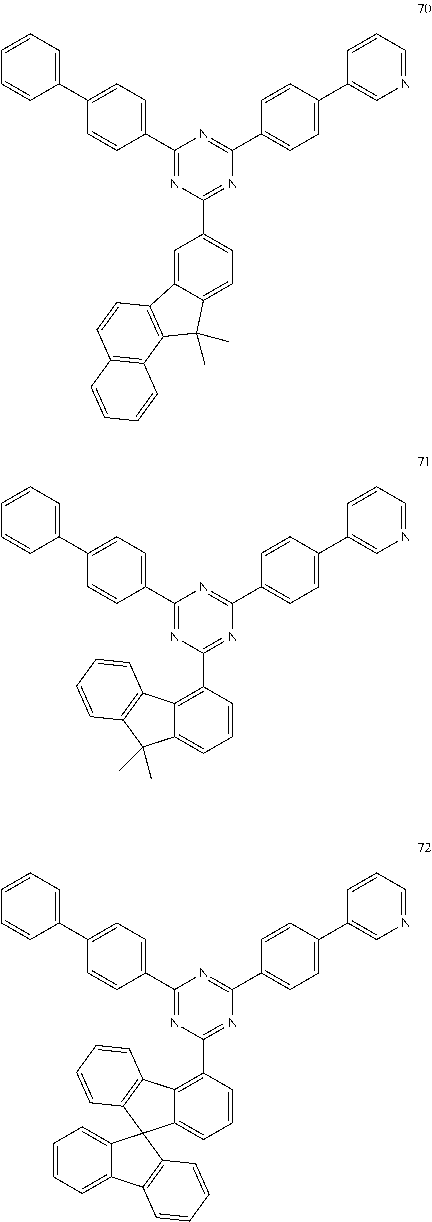 [SMILES: CC1(C)c2ccc(-c3nc(-c4ccc(-c5ccccc5)cc4)nc(-c4ccc(-c5cccnc5)cc4)n3)cc2-c2ccc3ccccc3c21 ; CC1(C)c2ccccc2-c2c(-c3nc(-c4ccc(-c5ccccc5)cc4)nc(-c4ccc(-c5cccnc5)cc4)n3)cccc21 ; c1ccc(-c2ccc(-c3nc(-c4ccc(-c5cccnc5)cc4)nc(-c4cccc5c4-c4ccccc4C54c5ccccc5-c5ccccc54)n3)cc2)cc1]

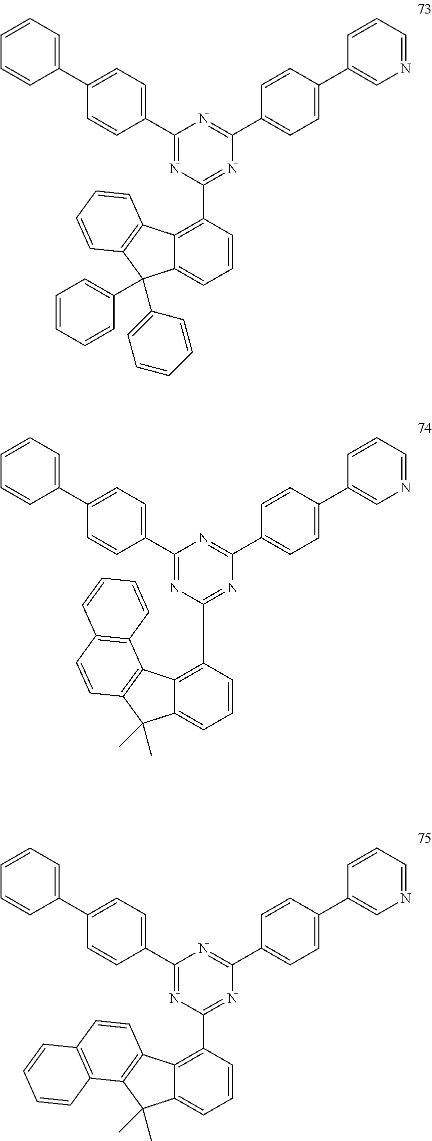 [SMILES: c1ccc(-c2ccc(-c3nc(-c4ccc(-c5cccnc5)cc4)nc(-c4cccc5c4-c4ccccc4C5(c4ccccc4)c4ccccc4)n3)cc2)cc1 ; CC1(C)c2cccc(-c3nc(-c4ccc(-c5ccccc5)cc4)nc(-c4ccc(-c5cccnc5)cc4)n3)c2-c2c1ccc1ccccc21 ; CC1(C)c2cccc(-c3nc(-c4ccc(-c5ccccc5)cc4)nc(-c4ccc(-c5cccnc5)cc4)n3)c2-c2ccc3ccccc3c21]

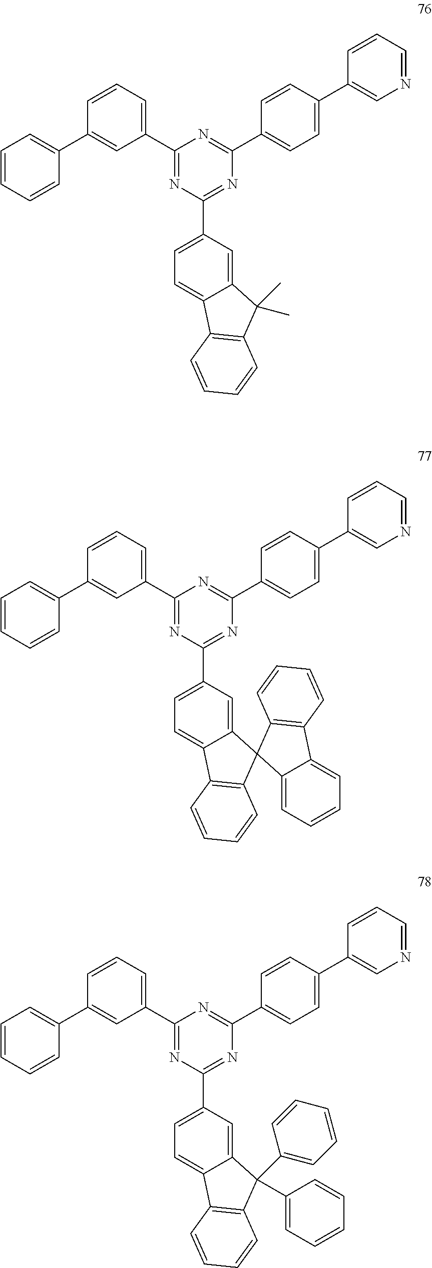 [SMILES: CC1(C)c2ccccc2-c2ccc(-c3nc(-c4ccc(-c5cccnc5)cc4)nc(-c4cccc(-c5ccccc5)c4)n3)cc21 ; c1ccc(-c2cccc(-c3nc(-c4ccc(-c5cccnc5)cc4)nc(-c4ccc5c(c4)C4(c6ccccc6-c6ccccc64)c4ccccc4-5)n3)c2)cc1 ; c1ccc(-c2cccc(-c3nc(-c4ccc(-c5cccnc5)cc4)nc(-c4ccc5c(c4)C(c4ccccc4)(c4ccccc4)c4ccccc4-5)n3)c2)cc1]

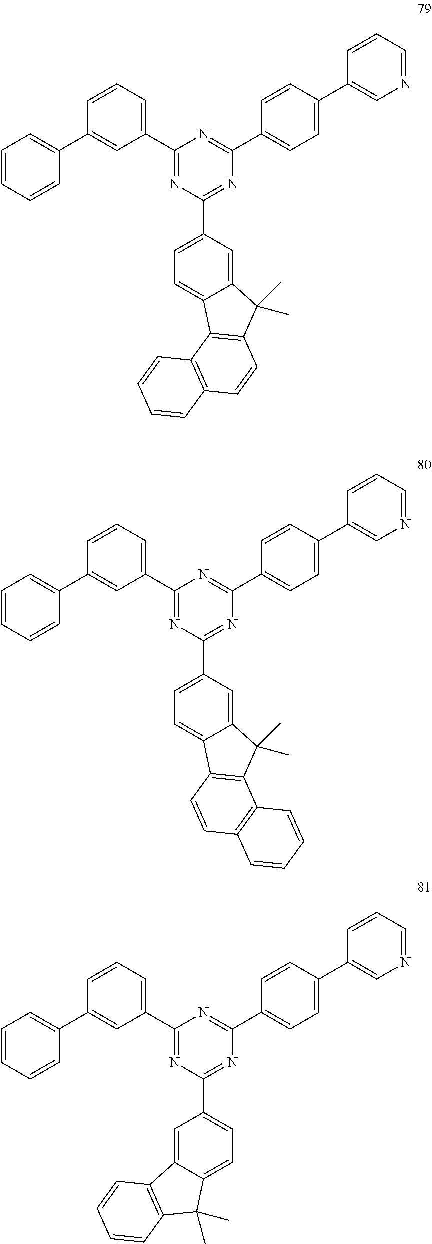 [SMILES: CC1(C)c2cc(-c3nc(-c4ccc(-c5cccnc5)cc4)nc(-c4cccc(-c5ccccc5)c4)n3)ccc2-c2c1ccc1ccccc21 ; CC1(C)c2cc(-c3nc(-c4ccc(-c5cccnc5)cc4)nc(-c4cccc(-c5ccccc5)c4)n3)ccc2-c2ccc3ccccc3c21 ; CC1(C)c2ccccc2-c2cc(-c3nc(-c4ccc(-c5cccnc5)cc4)nc(-c4cccc(-c5ccccc5)c4)n3)ccc21]

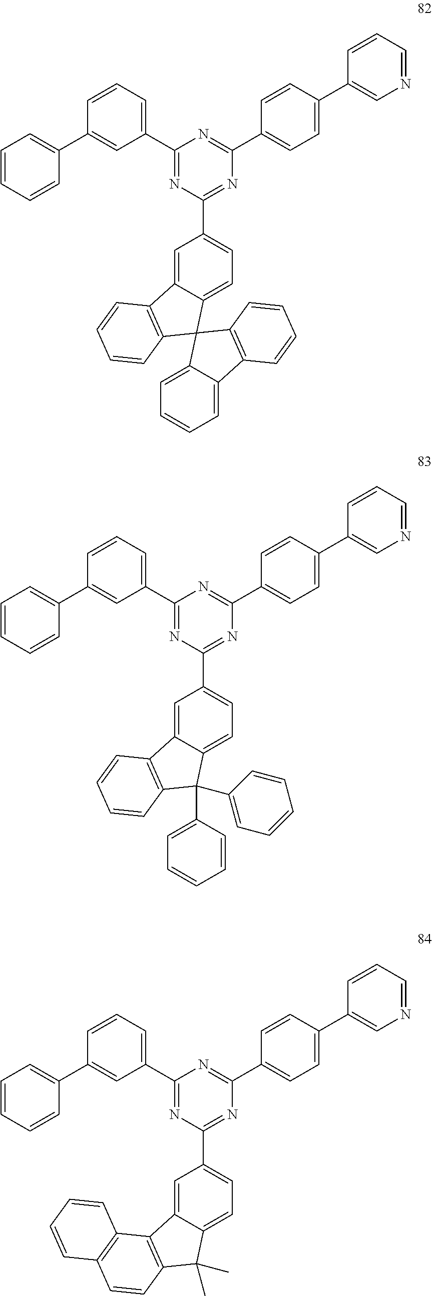 [SMILES: c1ccc(-c2cccc(-c3nc(-c4ccc(-c5cccnc5)cc4)nc(-c4ccc5c(c4)-c4ccccc4C54c5ccccc5-c5ccccc54)n3)c2)cc1 ; c1ccc(-c2cccc(-c3nc(-c4ccc(-c5cccnc5)cc4)nc(-c4ccc5c(c4)-c4ccccc4C5(c4ccccc4)c4ccccc4)n3)c2)cc1 ; CC1(C)c2ccc(-c3nc(-c4ccc(-c5cccnc5)cc4)nc(-c4cccc(-c5ccccc5)c4)n3)cc2-c2c1ccc1ccccc21]

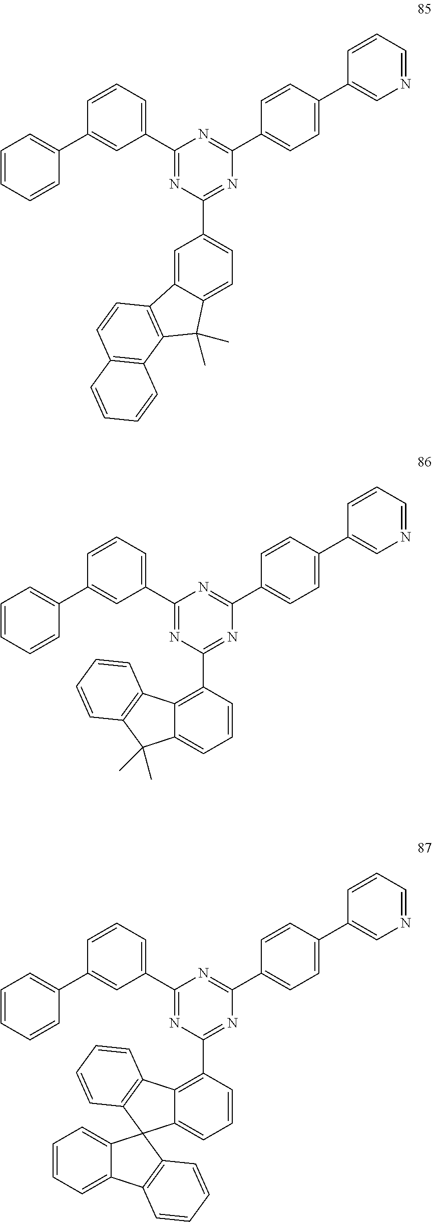 [SMILES: CC1(C)c2ccc(-c3nc(-c4ccc(-c5cccnc5)cc4)nc(-c4cccc(-c5ccccc5)c4)n3)cc2-c2ccc3ccccc3c21 ; CC1(C)c2ccccc2-c2c(-c3nc(-c4ccc(-c5cccnc5)cc4)nc(-c4cccc(-c5ccccc5)c4)n3)cccc21 ; c1ccc(-c2cccc(-c3nc(-c4ccc(-c5cccnc5)cc4)nc(-c4cccc5c4-c4ccccc4C54c5ccccc5-c5ccccc54)n3)c2)cc1]

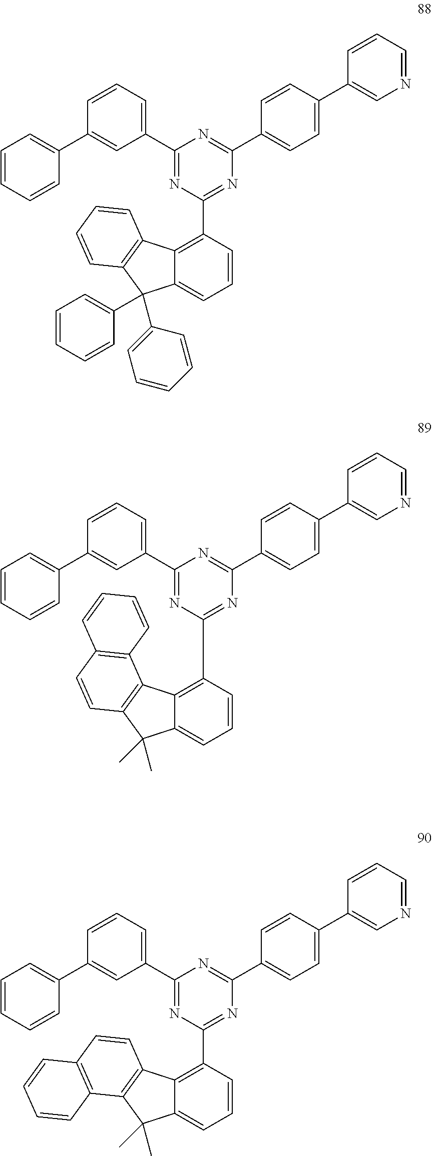 [SMILES: c1ccc(-c2cccc(-c3nc(-c4ccc(-c5cccnc5)cc4)nc(-c4cccc5c4-c4ccccc4C5(c4ccccc4)c4ccccc4)n3)c2)cc1 ; CC1(C)c2cccc(-c3nc(-c4ccc(-c5cccnc5)cc4)nc(-c4cccc(-c5ccccc5)c4)n3)c2-c2c1ccc1ccccc21 ; CC1(C)c2cccc(-c3nc(-c4ccc(-c5cccnc5)cc4)nc(-c4cccc(-c5ccccc5)c4)n3)c2-c2ccc3ccccc3c21]

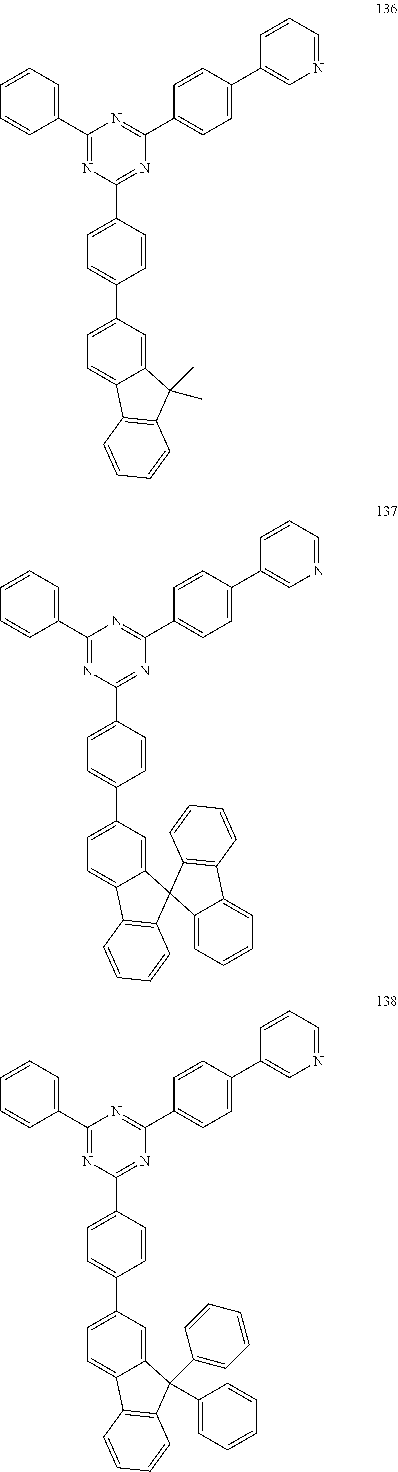 [SMILES: CC1(C)c2ccccc2-c2ccc(-c3ccc(-c4nc(-c5ccccc5)nc(-c5ccc(-c6cccnc6)cc5)n4)cc3)cc21 ; c1ccc(-c2nc(-c3ccc(-c4cccnc4)cc3)nc(-c3ccc(-c4ccc5c(c4)C4(c6ccccc6-c6ccccc64)c4ccccc4-5)cc3)n2)cc1 ; c1ccc(-c2nc(-c3ccc(-c4cccnc4)cc3)nc(-c3ccc(-c4ccc5c(c4)C(c4ccccc4)(c4ccccc4)c4ccccc4-5)cc3)n2)cc1]

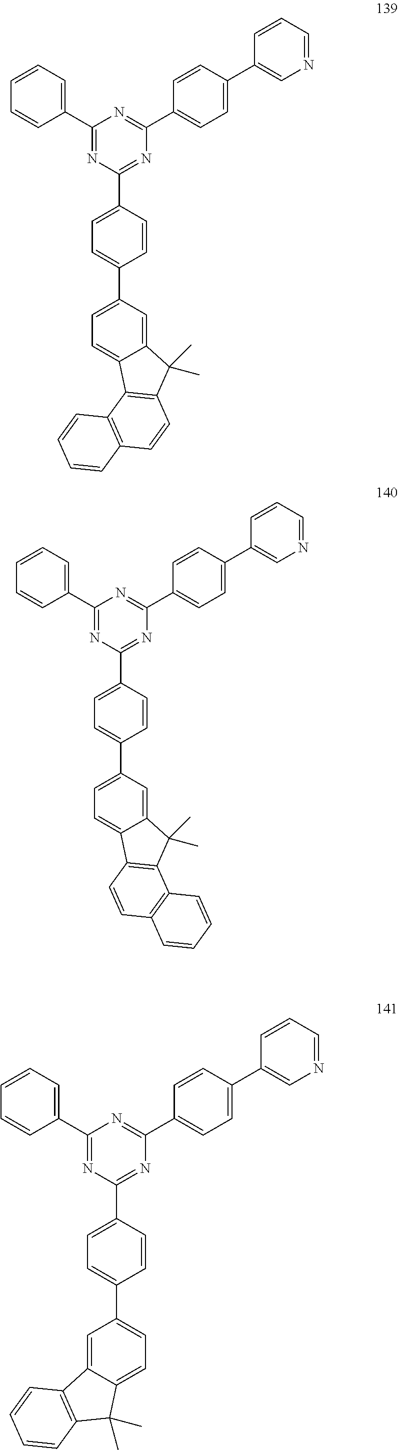 [SMILES: CC1(C)c2cc(-c3ccc(-c4nc(-c5ccccc5)nc(-c5ccc(-c6cccnc6)cc5)n4)cc3)ccc2-c2c1ccc1ccccc21 ; CC1(C)c2cc(-c3ccc(-c4nc(-c5ccccc5)nc(-c5ccc(-c6cccnc6)cc5)n4)cc3)ccc2-c2ccc3ccccc3c21 ; CC1(C)c2ccccc2-c2cc(-c3ccc(-c4nc(-c5ccccc5)nc(-c5ccc(-c6cccnc6)cc5)n4)cc3)ccc21]

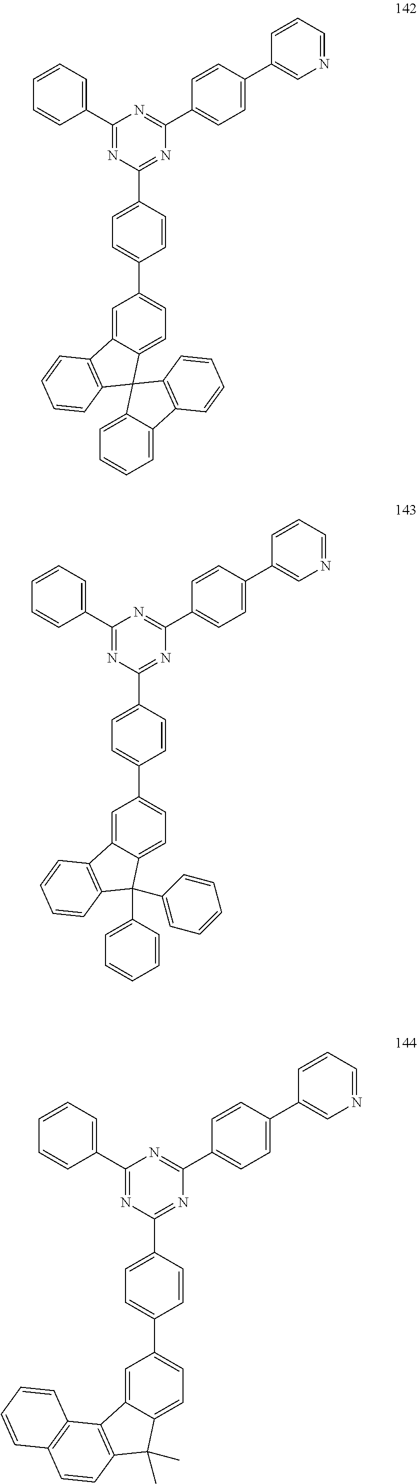 [SMILES: c1ccc(-c2nc(-c3ccc(-c4cccnc4)cc3)nc(-c3ccc(-c4ccc5c(c4)-c4ccccc4C54c5ccccc5-c5ccccc54)cc3)n2)cc1 ; c1ccc(-c2nc(-c3ccc(-c4cccnc4)cc3)nc(-c3ccc(-c4ccc5c(c4)-c4ccccc4C5(c4ccccc4)c4ccccc4)cc3)n2)cc1 ; CC1(C)c2ccc(-c3ccc(-c4nc(-c5ccccc5)nc(-c5ccc(-c6cccnc6)cc5)n4)cc3)cc2-c2c1ccc1ccccc21]

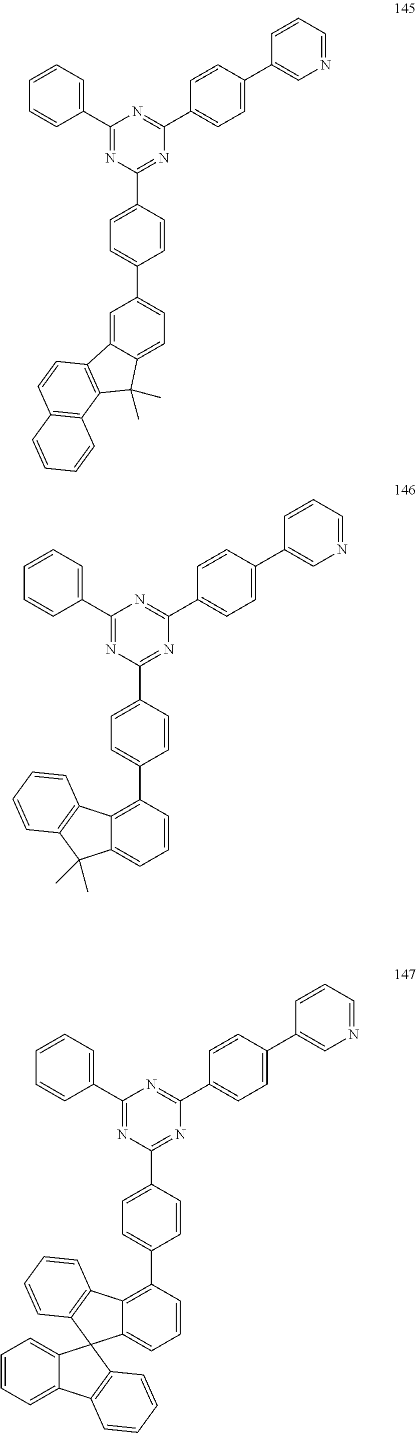 [SMILES: CC1(C)c2ccc(-c3ccc(-c4nc(-c5ccccc5)nc(-c5ccc(-c6cccnc6)cc5)n4)cc3)cc2-c2ccc3ccccc3c21 ; CC1(C)c2ccccc2-c2c(-c3ccc(-c4nc(-c5ccccc5)nc(-c5ccc(-c6cccnc6)cc5)n4)cc3)cccc21 ; c1ccc(-c2nc(-c3ccc(-c4cccnc4)cc3)nc(-c3ccc(-c4cccc5c4-c4ccccc4C54c5ccccc5-c5ccccc54)cc3)n2)cc1]

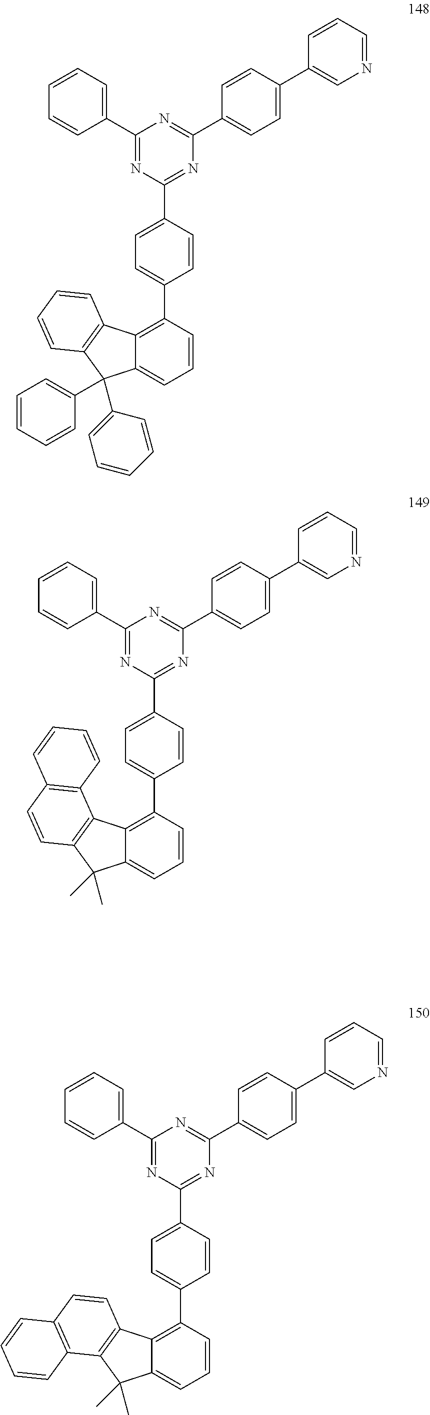 [SMILES: c1ccc(-c2nc(-c3ccc(-c4cccnc4)cc3)nc(-c3ccc(-c4cccc5c4-c4ccccc4C5(c4ccccc4)c4ccccc4)cc3)n2)cc1 ; CC1(C)c2cccc(-c3ccc(-c4nc(-c5ccccc5)nc(-c5ccc(-c6cccnc6)cc5)n4)cc3)c2-c2c1ccc1ccccc21 ; CC1(C)c2cccc(-c3ccc(-c4nc(-c5ccccc5)nc(-c5ccc(-c6cccnc6)cc5)n4)cc3)c2-c2ccc3ccccc3c21]

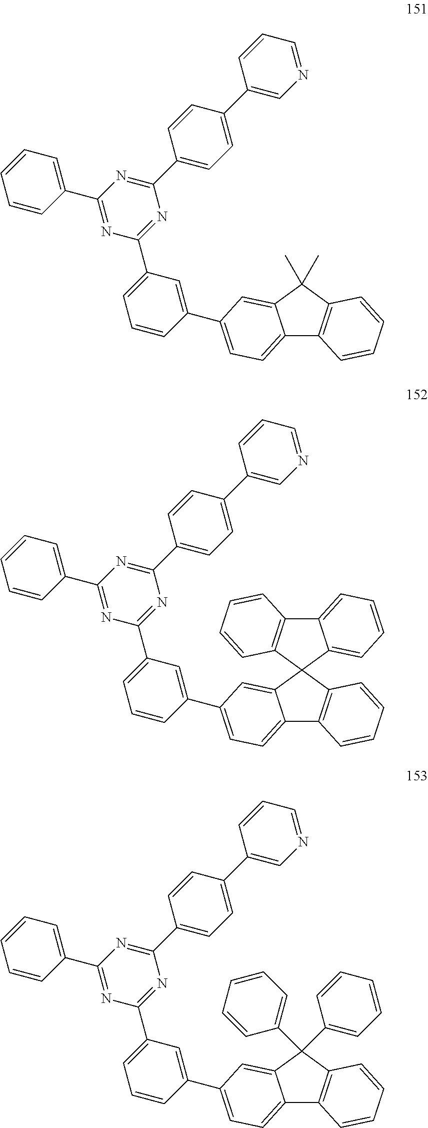 [SMILES: CC1(C)c2ccccc2-c2ccc(-c3cccc(-c4nc(-c5ccccc5)nc(-c5ccc(-c6cccnc6)cc5)n4)c3)cc21 ; c1ccc(-c2nc(-c3ccc(-c4cccnc4)cc3)nc(-c3cccc(-c4ccc5c(c4)C4(c6ccccc6-c6ccccc64)c4ccccc4-5)c3)n2)cc1 ; c1ccc(-c2nc(-c3ccc(-c4cccnc4)cc3)nc(-c3cccc(-c4ccc5c(c4)C(c4ccccc4)(c4ccccc4)c4ccccc4-5)c3)n2)cc1]

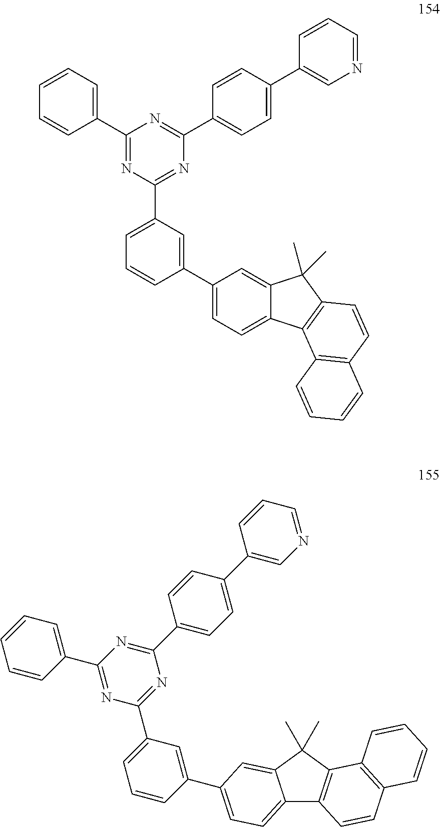 [SMILES: CC1(C)c2cc(-c3cccc(-c4nc(-c5ccccc5)nc(-c5ccc(-c6cccnc6)cc5)n4)c3)ccc2-c2c1ccc1ccccc21 ; CC1(C)c2cc(-c3cccc(-c4nc(-c5ccccc5)nc(-c5ccc(-c6cccnc6)cc5)n4)c3)ccc2-c2ccc3ccccc3c21]

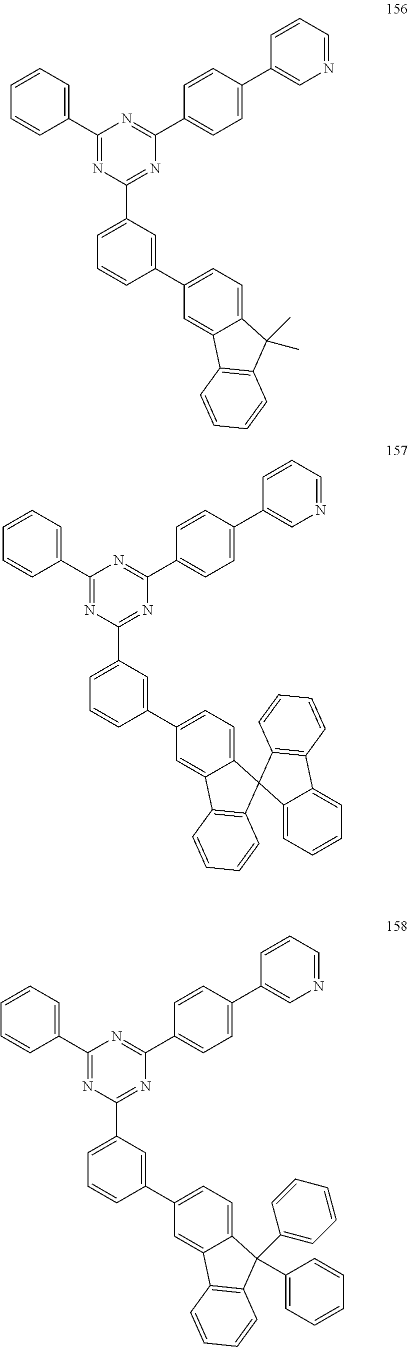 [SMILES: CC1(C)c2ccccc2-c2cc(-c3cccc(-c4nc(-c5ccccc5)nc(-c5ccc(-c6cccnc6)cc5)n4)c3)ccc21 ; c1ccc(-c2nc(-c3ccc(-c4cccnc4)cc3)nc(-c3cccc(-c4ccc5c(c4)-c4ccccc4C54c5ccccc5-c5ccccc54)c3)n2)cc1 ; c1ccc(-c2nc(-c3ccc(-c4cccnc4)cc3)nc(-c3cccc(-c4ccc5c(c4)-c4ccccc4C5(c4ccccc4)c4ccccc4)c3)n2)cc1]

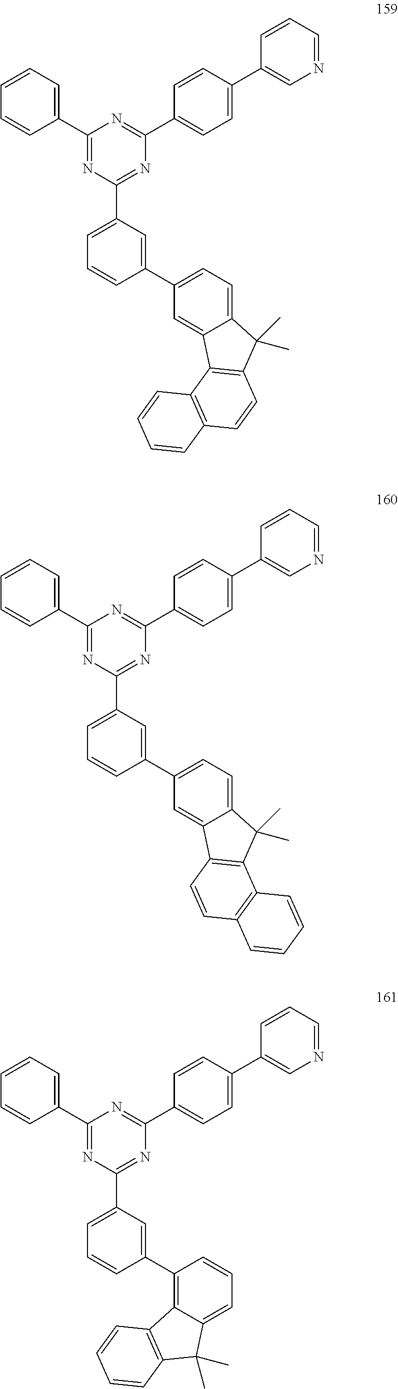 [SMILES: CC1(C)c2ccc(-c3cccc(-c4nc(-c5ccccc5)nc(-c5ccc(-c6cccnc6)cc5)n4)c3)cc2-c2c1ccc1ccccc21 ; CC1(C)c2ccc(-c3cccc(-c4nc(-c5ccccc5)nc(-c5ccc(-c6cccnc6)cc5)n4)c3)cc2-c2ccc3ccccc3c21 ; CC1(C)c2ccccc2-c2c(-c3cccc(-c4nc(-c5ccccc5)nc(-c5ccc(-c6cccnc6)cc5)n4)c3)cccc21]

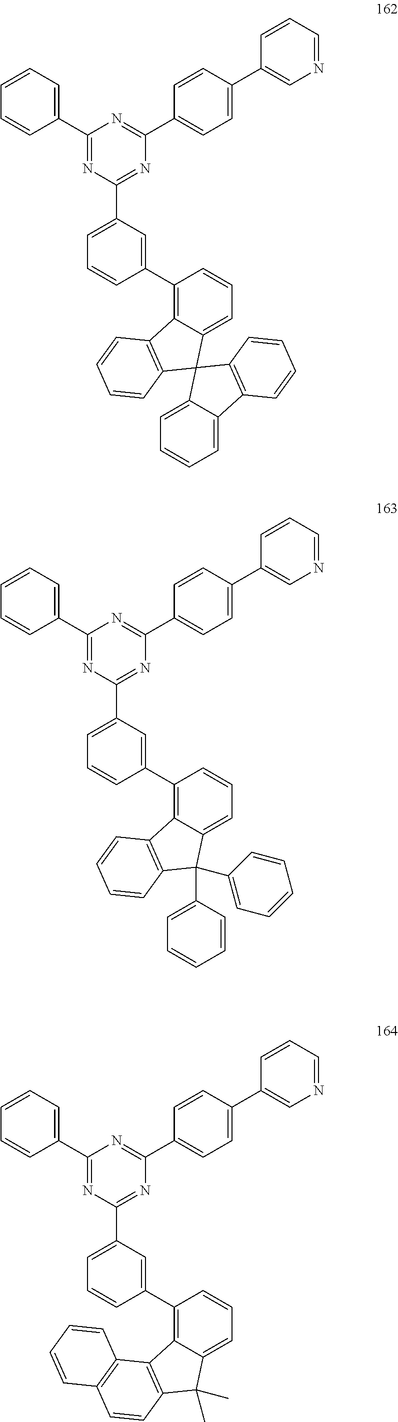 [SMILES: c1ccc(-c2nc(-c3ccc(-c4cccnc4)cc3)nc(-c3cccc(-c4cccc5c4-c4ccccc4C54c5ccccc5-c5ccccc54)c3)n2)cc1 ; c1ccc(-c2nc(-c3ccc(-c4cccnc4)cc3)nc(-c3cccc(-c4cccc5c4-c4ccccc4C5(c4ccccc4)c4ccccc4)c3)n2)cc1 ; CC1(C)c2cccc(-c3cccc(-c4nc(-c5ccccc5)nc(-c5ccc(-c6cccnc6)cc5)n4)c3)c2-c2c1ccc1ccccc21]

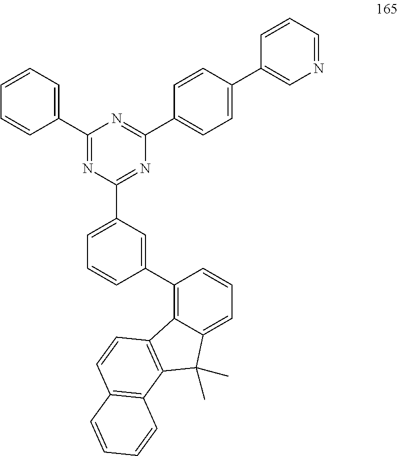 [SMILES: CC1(C)c2cccc(-c3cccc(-c4nc(-c5ccccc5)nc(-c5ccc(-c6cccnc6)cc5)n4)c3)c2-c2ccc3ccccc3c21]

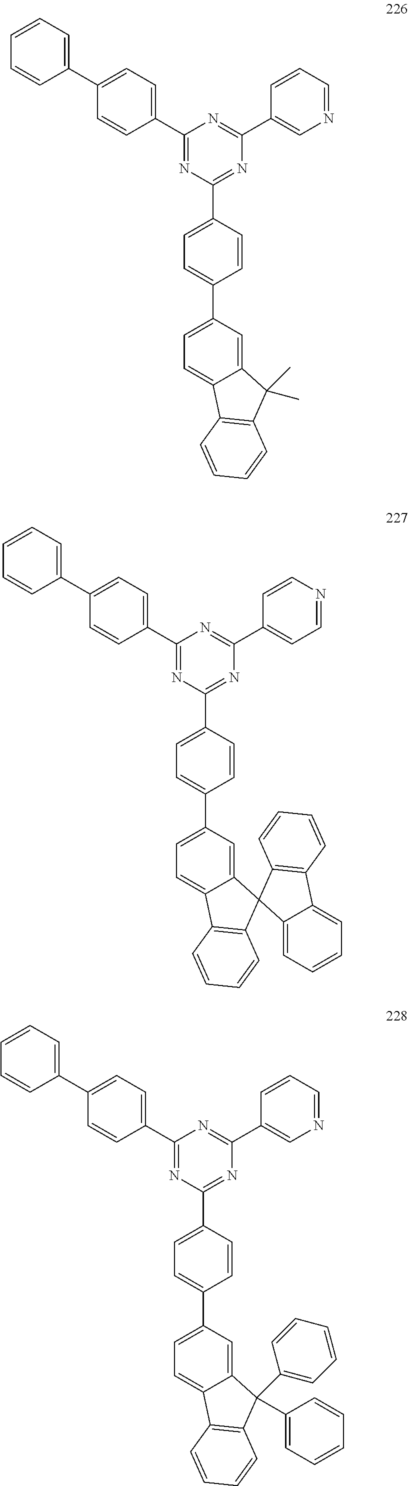 [SMILES: CC1(C)c2ccccc2-c2ccc(-c3ccc(-c4nc(-c5ccc(-c6ccccc6)cc5)nc(-c5cccnc5)n4)cc3)cc21 ; c1ccc(-c2ccc(-c3nc(-c4ccncc4)nc(-c4ccc(-c5ccc6c(c5)C5(c7ccccc7-c7ccccc75)c5ccccc5-6)cc4)n3)cc2)cc1 ; c1ccc(-c2ccc(-c3nc(-c4ccc(-c5ccc6c(c5)C(c5ccccc5)(c5ccccc5)c5ccccc5-6)cc4)nc(-c4cccnc4)n3)cc2)cc1]

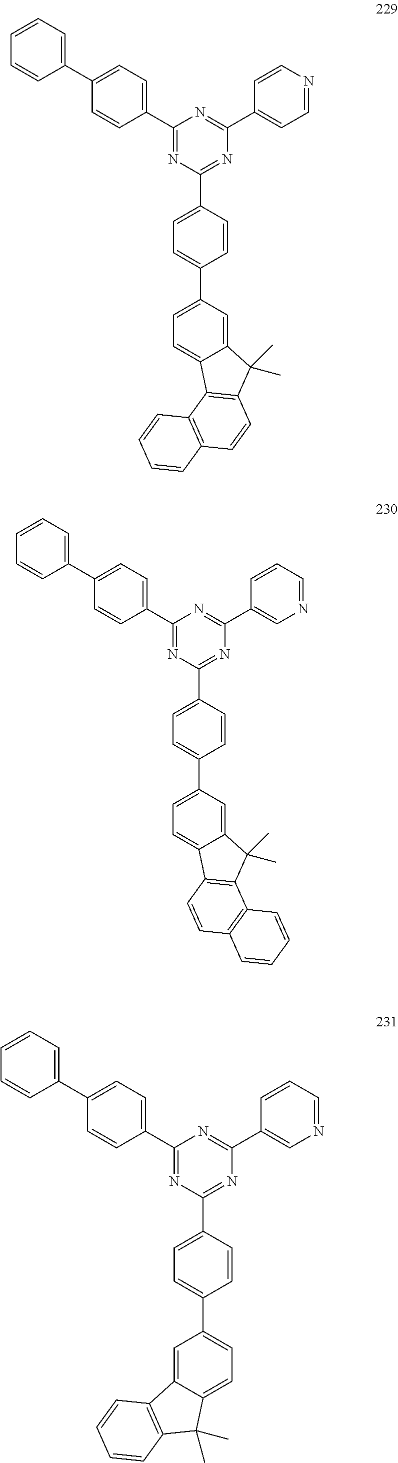 [SMILES: CC1(C)c2cc(-c3ccc(-c4nc(-c5ccncc5)nc(-c5ccc(-c6ccccc6)cc5)n4)cc3)ccc2-c2c1ccc1ccccc21 ; CC1(C)c2cc(-c3ccc(-c4nc(-c5ccc(-c6ccccc6)cc5)nc(-c5cccnc5)n4)cc3)ccc2-c2ccc3ccccc3c21 ; CC1(C)c2ccccc2-c2cc(-c3ccc(-c4nc(-c5ccc(-c6ccccc6)cc5)nc(-c5cccnc5)n4)cc3)ccc21]

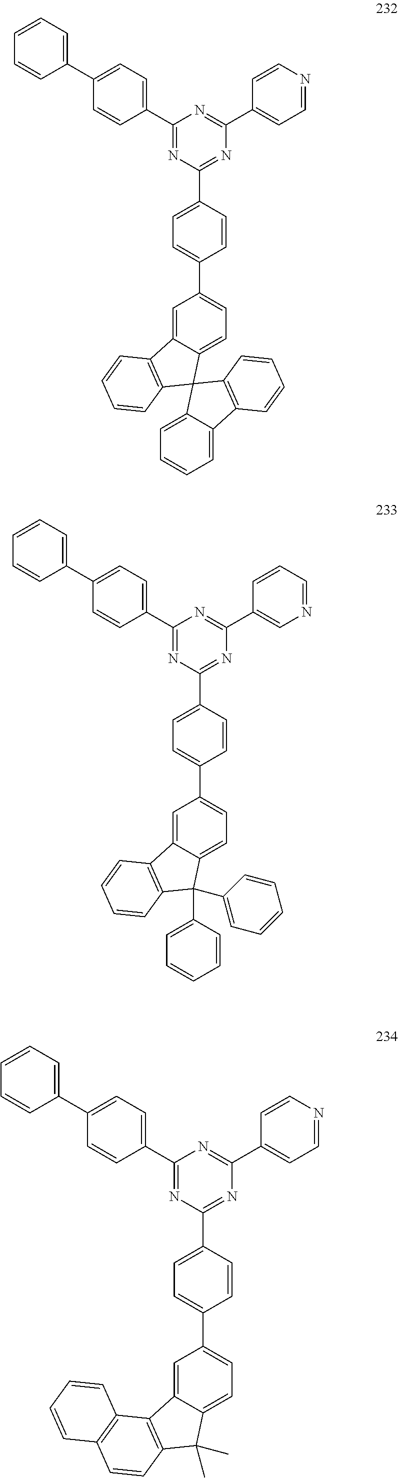 [SMILES: c1ccc(-c2ccc(-c3nc(-c4ccncc4)nc(-c4ccc(-c5ccc6c(c5)-c5ccccc5C65c6ccccc6-c6ccccc65)cc4)n3)cc2)cc1 ; c1ccc(-c2ccc(-c3nc(-c4ccc(-c5ccc6c(c5)-c5ccccc5C6(c5ccccc5)c5ccccc5)cc4)nc(-c4cccnc4)n3)cc2)cc1 ; CC1(C)c2ccc(-c3ccc(-c4nc(-c5ccncc5)nc(-c5ccc(-c6ccccc6)cc5)n4)cc3)cc2-c2c1ccc1ccccc21]

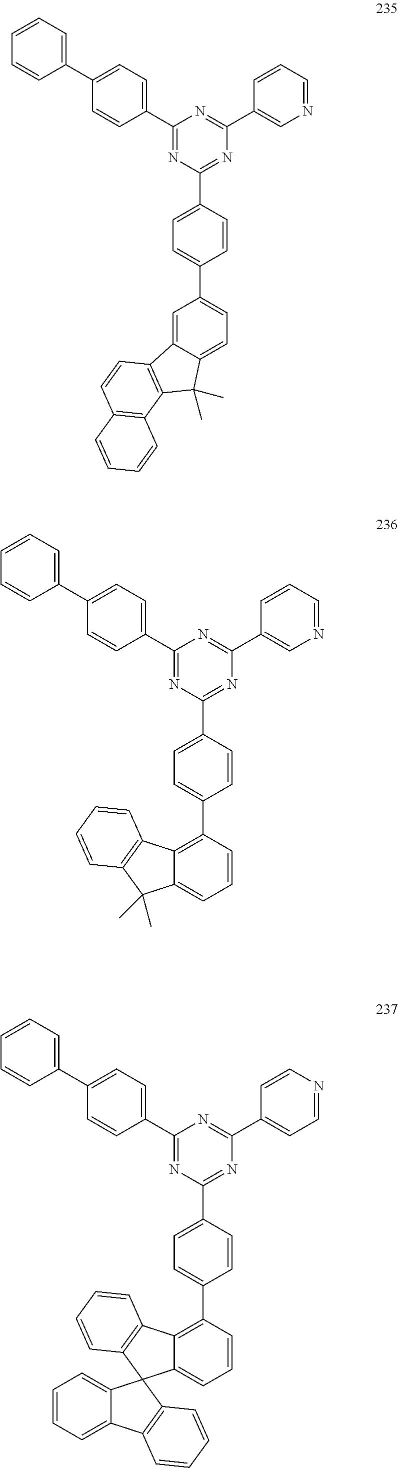 [SMILES: CC1(C)c2ccc(-c3ccc(-c4nc(-c5ccc(-c6ccccc6)cc5)nc(-c5cccnc5)n4)cc3)cc2-c2ccc3ccccc3c21 ; CC1(C)c2ccccc2-c2c(-c3ccc(-c4nc(-c5ccc(-c6ccccc6)cc5)nc(-c5cccnc5)n4)cc3)cccc21 ; c1ccc(-c2ccc(-c3nc(-c4ccncc4)nc(-c4ccc(-c5cccc6c5-c5ccccc5C65c6ccccc6-c6ccccc65)cc4)n3)cc2)cc1]

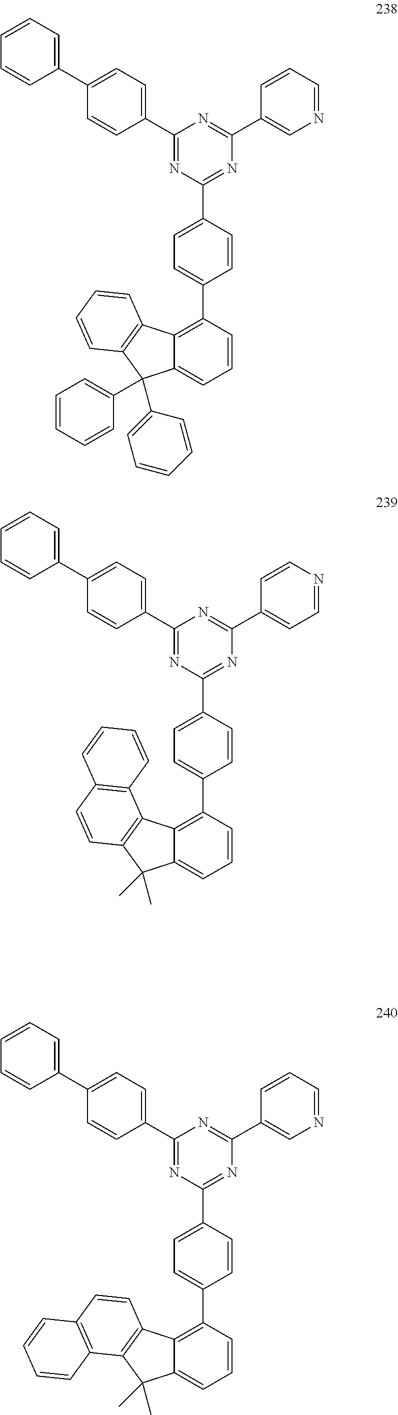 [SMILES: c1ccc(-c2ccc(-c3nc(-c4ccc(-c5cccc6c5-c5ccccc5C6(c5ccccc5)c5ccccc5)cc4)nc(-c4cccnc4)n3)cc2)cc1 ; CC1(C)c2cccc(-c3ccc(-c4nc(-c5ccncc5)nc(-c5ccc(-c6ccccc6)cc5)n4)cc3)c2-c2c1ccc1ccccc21 ; CC1(C)c2cccc(-c3ccc(-c4nc(-c5ccc(-c6ccccc6)cc5)nc(-c5cccnc5)n4)cc3)c2-c2ccc3ccccc3c21]

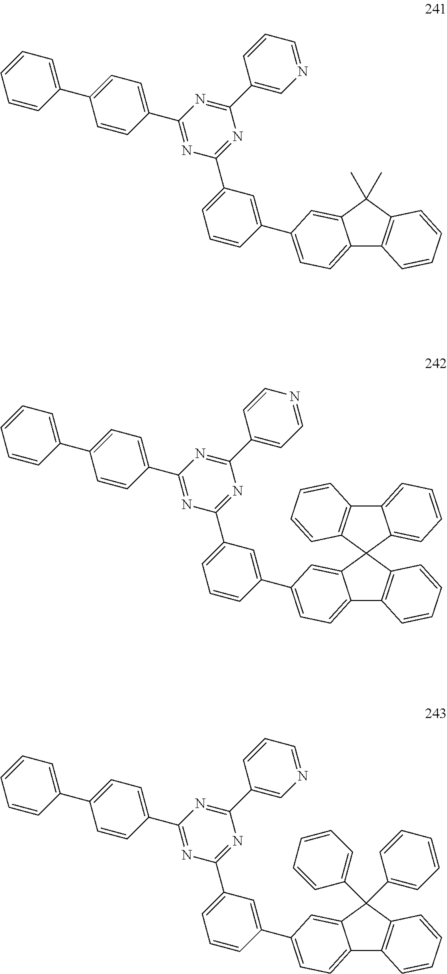 [SMILES: CC1(C)c2ccccc2-c2ccc(-c3cccc(-c4nc(-c5ccc(-c6ccccc6)cc5)nc(-c5cccnc5)n4)c3)cc21 ; c1ccc(-c2ccc(-c3nc(-c4ccncc4)nc(-c4cccc(-c5ccc6c(c5)C5(c7ccccc7-c7ccccc75)c5ccccc5-6)c4)n3)cc2)cc1 ; c1ccc(-c2ccc(-c3nc(-c4cccnc4)nc(-c4cccc(-c5ccc6c(c5)C(c5ccccc5)(c5ccccc5)c5ccccc5-6)c4)n3)cc2)cc1]

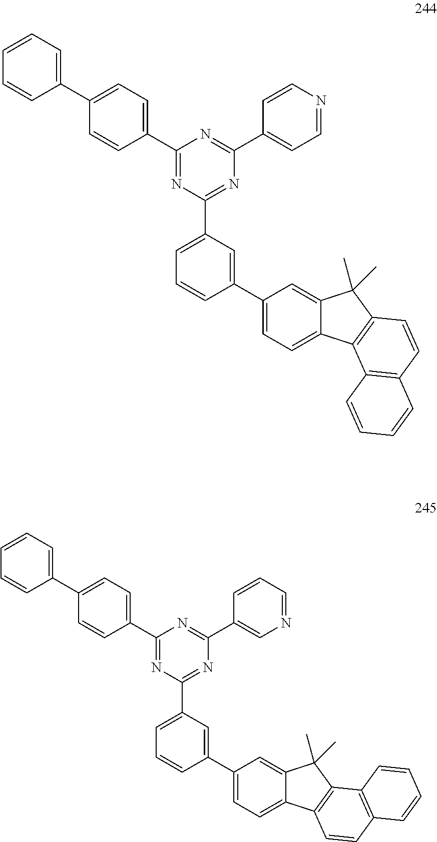 [SMILES: CC1(C)c2cc(-c3cccc(-c4nc(-c5ccncc5)nc(-c5ccc(-c6ccccc6)cc5)n4)c3)ccc2-c2c1ccc1ccccc21 ; CC1(C)c2cc(-c3cccc(-c4nc(-c5ccc(-c6ccccc6)cc5)nc(-c5cccnc5)n4)c3)ccc2-c2ccc3ccccc3c21]

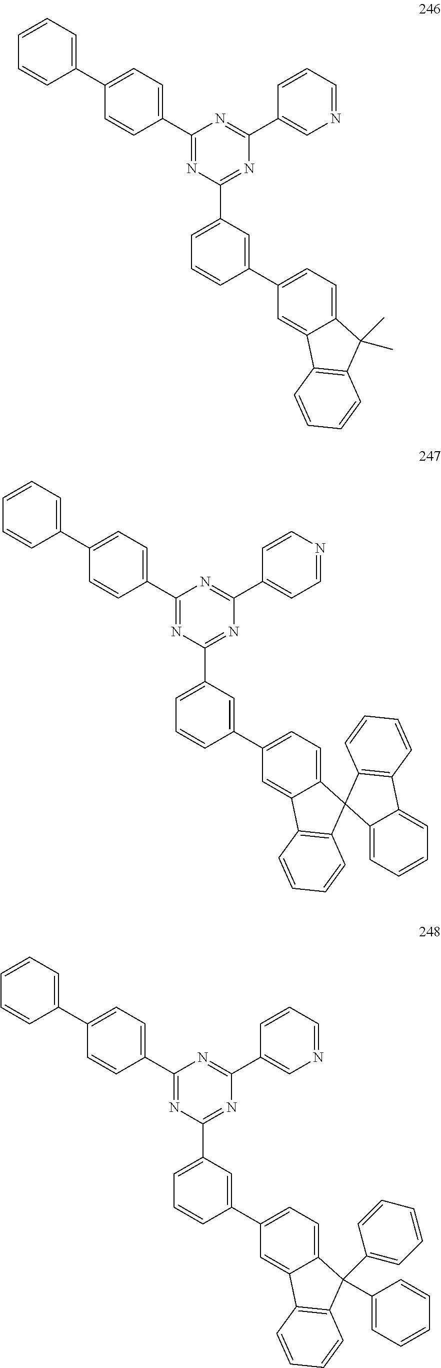 [SMILES: CC1(C)c2ccccc2-c2cc(-c3cccc(-c4nc(-c5ccc(-c6ccccc6)cc5)nc(-c5cccnc5)n4)c3)ccc21 ; c1ccc(-c2ccc(-c3nc(-c4ccncc4)nc(-c4cccc(-c5ccc6c(c5)-c5ccccc5C65c6ccccc6-c6ccccc65)c4)n3)cc2)cc1 ; c1ccc(-c2ccc(-c3nc(-c4cccnc4)nc(-c4cccc(-c5ccc6c(c5)-c5ccccc5C6(c5ccccc5)c5ccccc5)c4)n3)cc2)cc1]

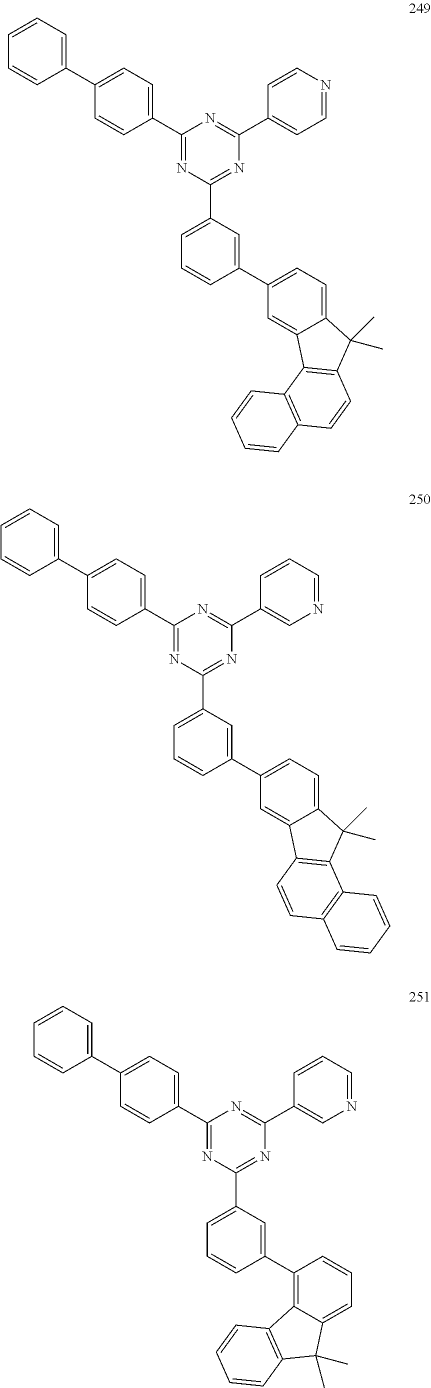 [SMILES: CC1(C)c2ccc(-c3cccc(-c4nc(-c5ccncc5)nc(-c5ccc(-c6ccccc6)cc5)n4)c3)cc2-c2c1ccc1ccccc21 ; CC1(C)c2ccc(-c3cccc(-c4nc(-c5ccc(-c6ccccc6)cc5)nc(-c5cccnc5)n4)c3)cc2-c2ccc3ccccc3c21 ; CC1(C)c2ccccc2-c2c(-c3cccc(-c4nc(-c5ccc(-c6ccccc6)cc5)nc(-c5cccnc5)n4)c3)cccc21]

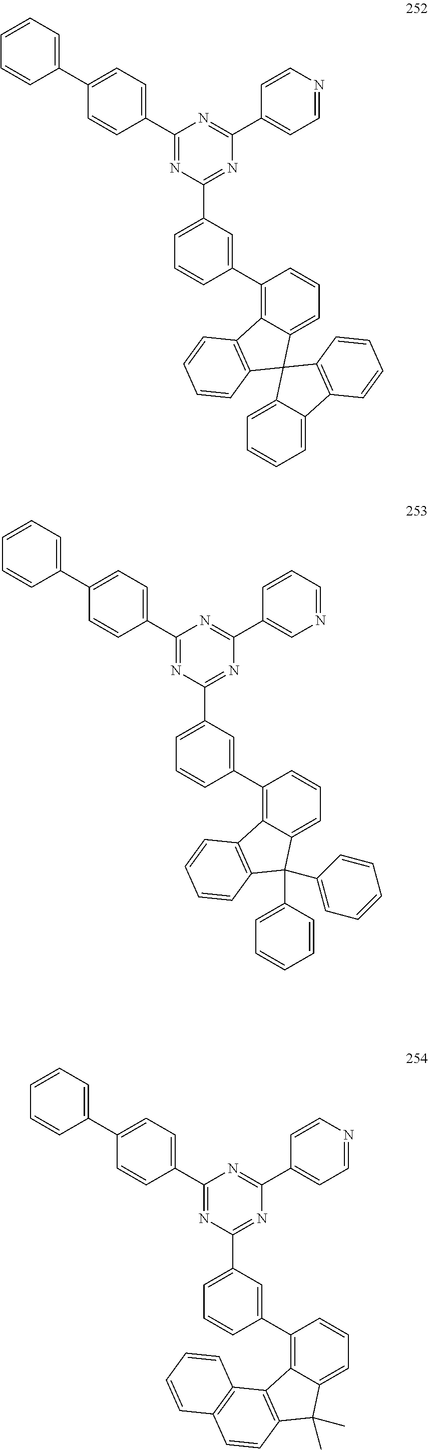 [SMILES: c1ccc(-c2ccc(-c3nc(-c4ccncc4)nc(-c4cccc(-c5cccc6c5-c5ccccc5C65c6ccccc6-c6ccccc65)c4)n3)cc2)cc1 ; c1ccc(-c2ccc(-c3nc(-c4cccnc4)nc(-c4cccc(-c5cccc6c5-c5ccccc5C6(c5ccccc5)c5ccccc5)c4)n3)cc2)cc1 ; CC1(C)c2cccc(-c3cccc(-c4nc(-c5ccncc5)nc(-c5ccc(-c6ccccc6)cc5)n4)c3)c2-c2c1ccc1ccccc21]

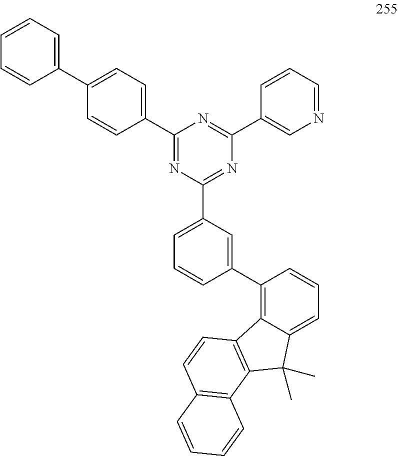 [SMILES: CC1(C)c2cccc(-c3cccc(-c4nc(-c5ccc(-c6ccccc6)cc5)nc(-c5cccnc5)n4)c3)c2-c2ccc3ccccc3c21]

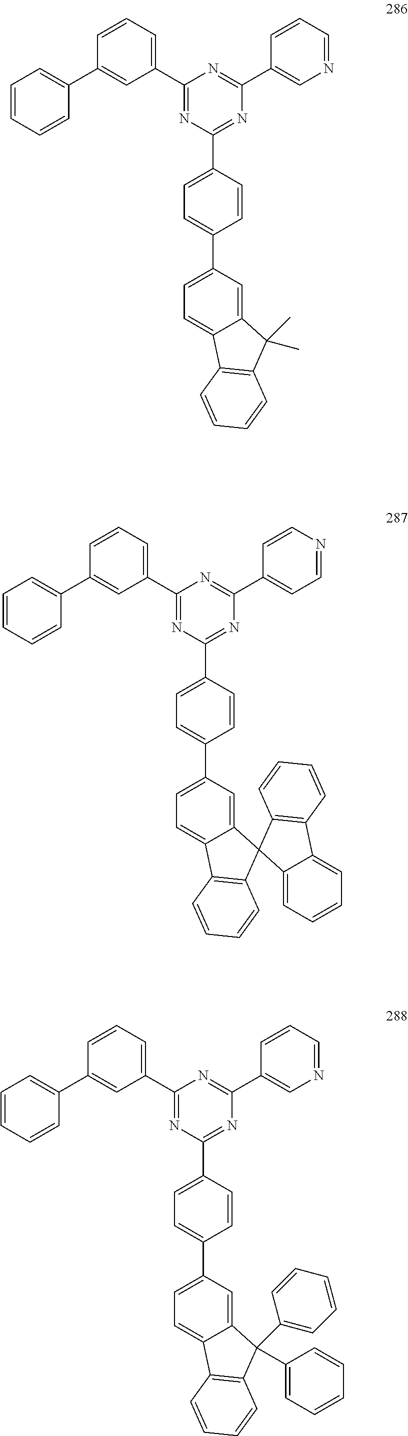 [SMILES: CC1(C)c2ccccc2-c2ccc(-c3ccc(-c4nc(-c5cccnc5)nc(-c5cccc(-c6ccccc6)c5)n4)cc3)cc21 ; c1ccc(-c2cccc(-c3nc(-c4ccncc4)nc(-c4ccc(-c5ccc6c(c5)C5(c7ccccc7-c7ccccc75)c5ccccc5-6)cc4)n3)c2)cc1 ; c1ccc(-c2cccc(-c3nc(-c4ccc(-c5ccc6c(c5)C(c5ccccc5)(c5ccccc5)c5ccccc5-6)cc4)nc(-c4cccnc4)n3)c2)cc1]

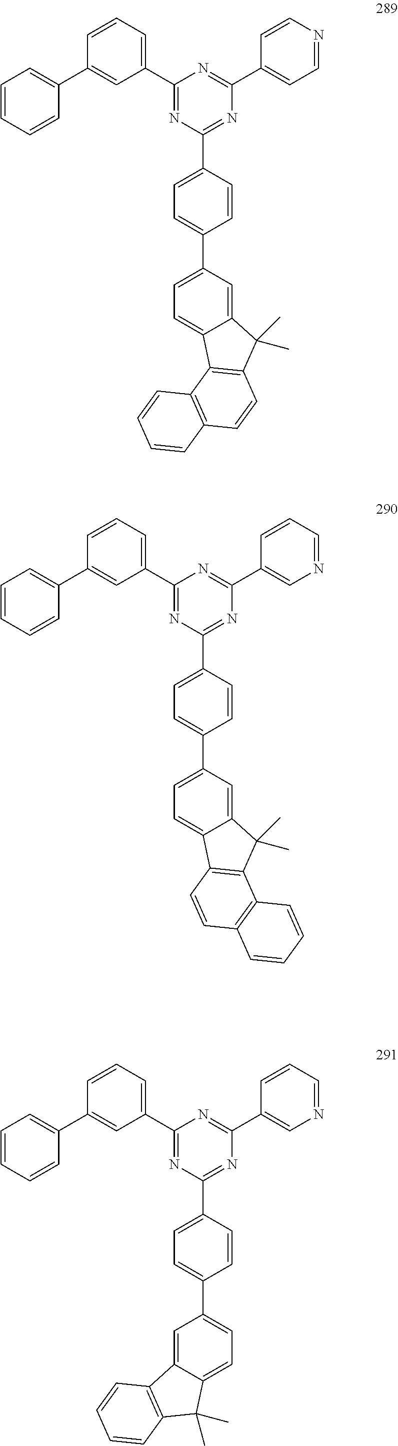 [SMILES: CC1(C)c2cc(-c3ccc(-c4nc(-c5ccncc5)nc(-c5cccc(-c6ccccc6)c5)n4)cc3)ccc2-c2c1ccc1ccccc21 ; CC1(C)c2cc(-c3ccc(-c4nc(-c5cccnc5)nc(-c5cccc(-c6ccccc6)c5)n4)cc3)ccc2-c2ccc3ccccc3c21 ; CC1(C)c2ccccc2-c2cc(-c3ccc(-c4nc(-c5cccnc5)nc(-c5cccc(-c6ccccc6)c5)n4)cc3)ccc21]

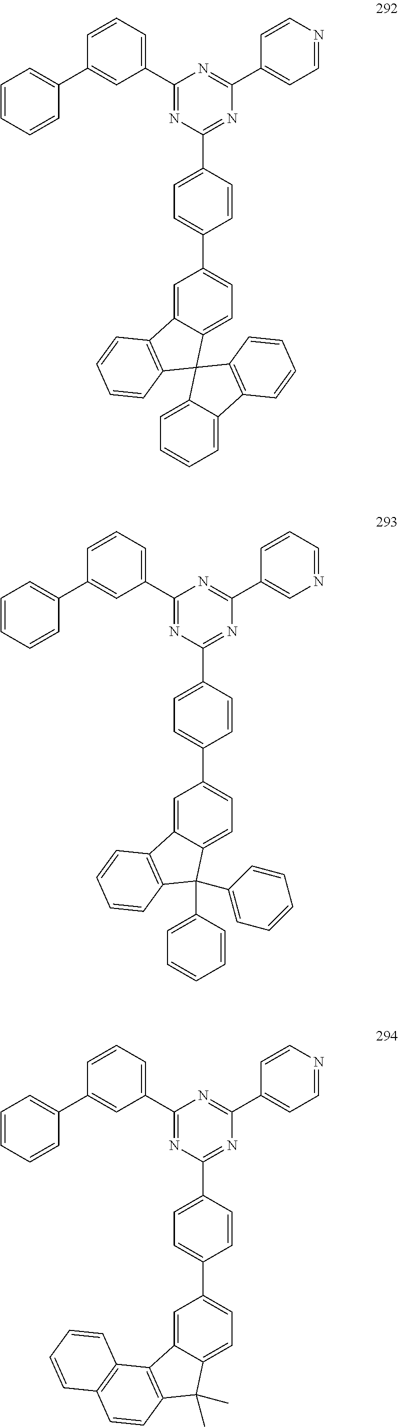 [SMILES: c1ccc(-c2cccc(-c3nc(-c4ccncc4)nc(-c4ccc(-c5ccc6c(c5)-c5ccccc5C65c6ccccc6-c6ccccc65)cc4)n3)c2)cc1 ; c1ccc(-c2cccc(-c3nc(-c4ccc(-c5ccc6c(c5)-c5ccccc5C6(c5ccccc5)c5ccccc5)cc4)nc(-c4cccnc4)n3)c2)cc1 ; CC1(C)c2ccc(-c3ccc(-c4nc(-c5ccncc5)nc(-c5cccc(-c6ccccc6)c5)n4)cc3)cc2-c2c1ccc1ccccc21]

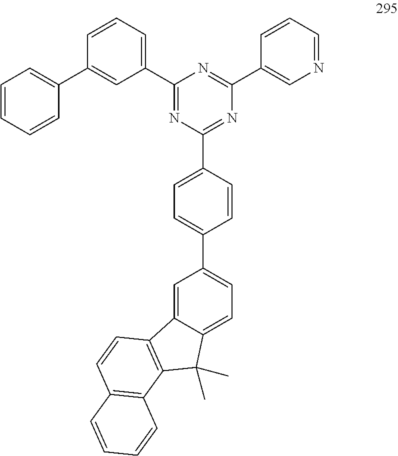 [SMILES: CC1(C)c2ccc(-c3ccc(-c4nc(-c5cccnc5)nc(-c5cccc(-c6ccccc6)c5)n4)cc3)cc2-c2ccc3ccccc3c21]

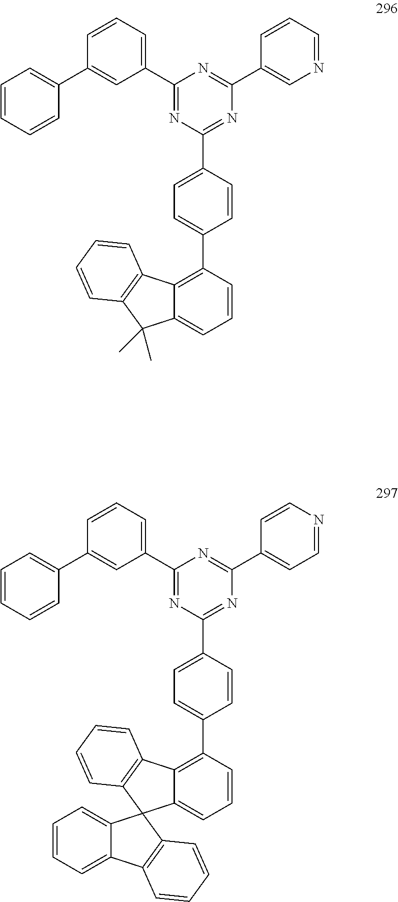 [SMILES: CC1(C)c2ccccc2-c2c(-c3ccc(-c4nc(-c5cccnc5)nc(-c5cccc(-c6ccccc6)c5)n4)cc3)cccc21 ; c1ccc(-c2cccc(-c3nc(-c4ccncc4)nc(-c4ccc(-c5cccc6c5-c5ccccc5C65c6ccccc6-c6ccccc65)cc4)n3)c2)cc1]

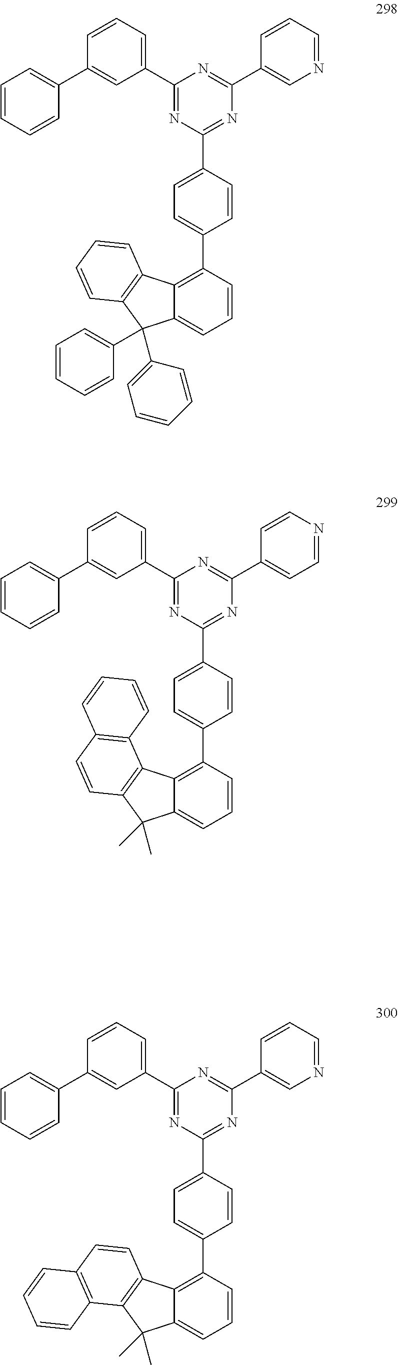 [SMILES: c1ccc(-c2cccc(-c3nc(-c4ccc(-c5cccc6c5-c5ccccc5C6(c5ccccc5)c5ccccc5)cc4)nc(-c4cccnc4)n3)c2)cc1 ; CC1(C)c2cccc(-c3ccc(-c4nc(-c5ccncc5)nc(-c5cccc(-c6ccccc6)c5)n4)cc3)c2-c2c1ccc1ccccc21 ; CC1(C)c2cccc(-c3ccc(-c4nc(-c5cccnc5)nc(-c5cccc(-c6ccccc6)c5)n4)cc3)c2-c2ccc3ccccc3c21]

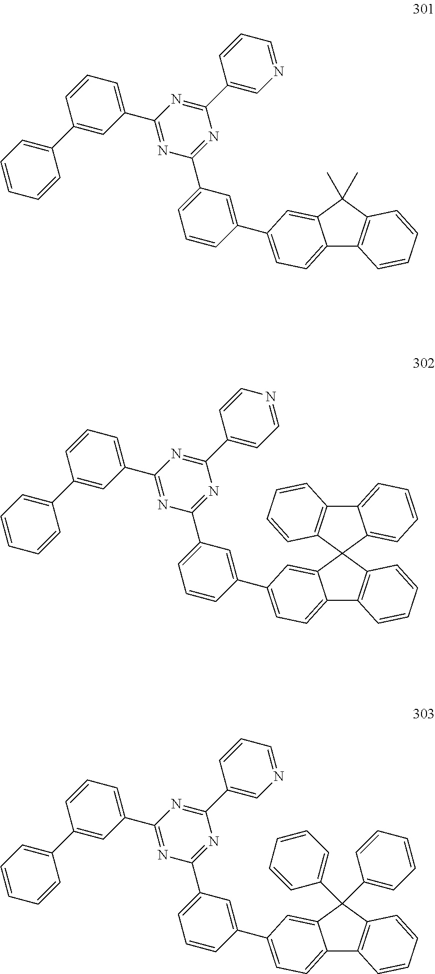 [SMILES: CC1(C)c2ccccc2-c2ccc(-c3cccc(-c4nc(-c5cccnc5)nc(-c5cccc(-c6ccccc6)c5)n4)c3)cc21 ; c1ccc(-c2cccc(-c3nc(-c4ccncc4)nc(-c4cccc(-c5ccc6c(c5)C5(c7ccccc7-c7ccccc75)c5ccccc5-6)c4)n3)c2)cc1 ; c1ccc(-c2cccc(-c3nc(-c4cccnc4)nc(-c4cccc(-c5ccc6c(c5)C(c5ccccc5)(c5ccccc5)c5ccccc5-6)c4)n3)c2)cc1]

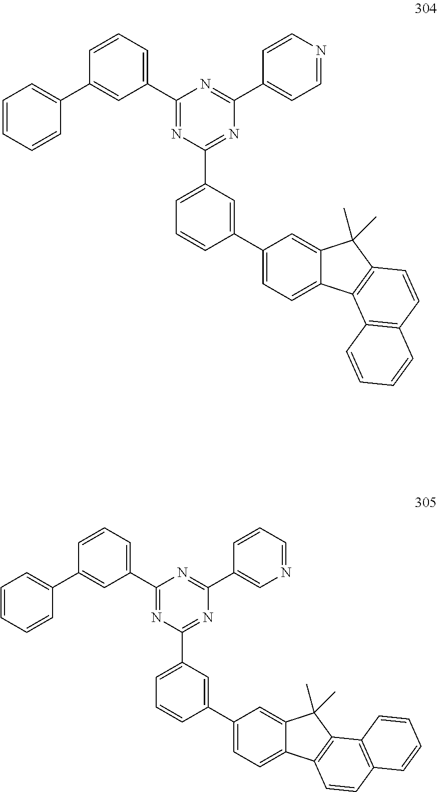 [SMILES: CC1(C)c2cc(-c3cccc(-c4nc(-c5ccncc5)nc(-c5cccc(-c6ccccc6)c5)n4)c3)ccc2-c2c1ccc1ccccc21 ; CC1(C)c2cc(-c3cccc(-c4nc(-c5cccnc5)nc(-c5cccc(-c6ccccc6)c5)n4)c3)ccc2-c2ccc3ccccc3c21]

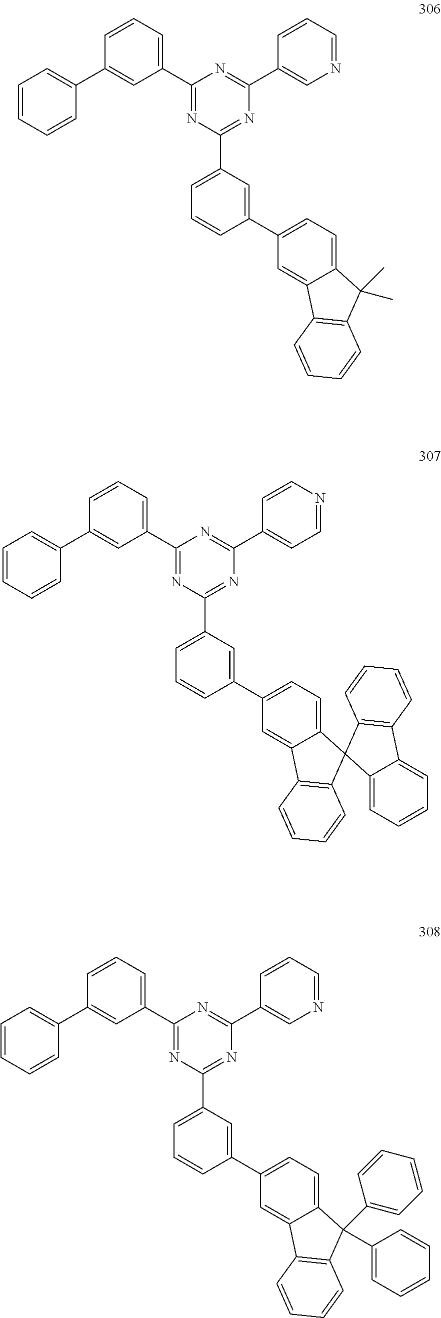 [SMILES: CC1(C)c2ccccc2-c2cc(-c3cccc(-c4nc(-c5cccnc5)nc(-c5cccc(-c6ccccc6)c5)n4)c3)ccc21 ; c1ccc(-c2cccc(-c3nc(-c4ccncc4)nc(-c4cccc(-c5ccc6c(c5)-c5ccccc5C65c6ccccc6-c6ccccc65)c4)n3)c2)cc1 ; c1ccc(-c2cccc(-c3nc(-c4cccnc4)nc(-c4cccc(-c5ccc6c(c5)-c5ccccc5C6(c5ccccc5)c5ccccc5)c4)n3)c2)cc1]

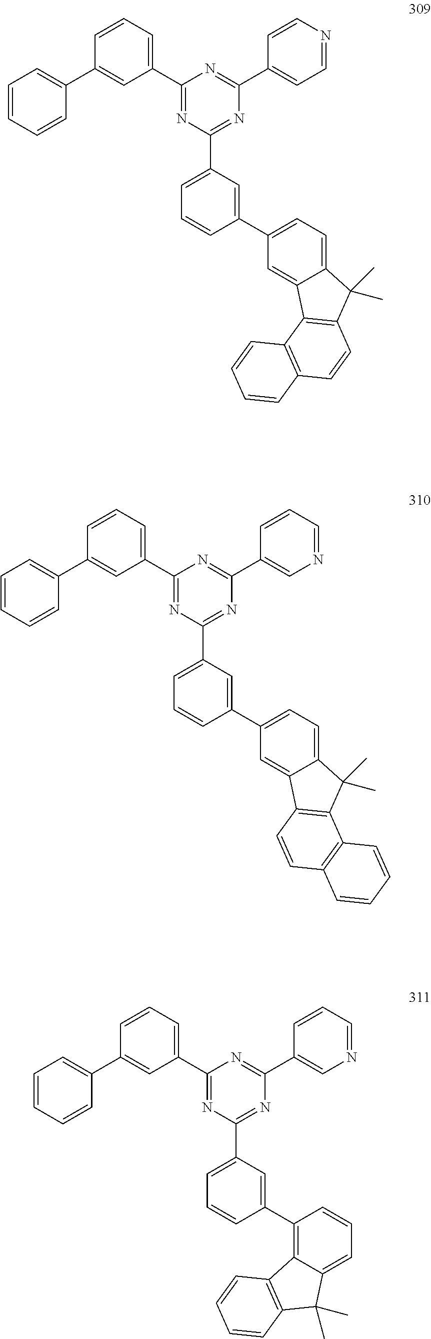 [SMILES: CC1(C)c2ccc(-c3cccc(-c4nc(-c5ccncc5)nc(-c5cccc(-c6ccccc6)c5)n4)c3)cc2-c2c1ccc1ccccc21 ; CC1(C)c2ccc(-c3cccc(-c4nc(-c5cccnc5)nc(-c5cccc(-c6ccccc6)c5)n4)c3)cc2-c2ccc3ccccc3c21 ; CC1(C)c2ccccc2-c2c(-c3cccc(-c4nc(-c5cccnc5)nc(-c5cccc(-c6ccccc6)c5)n4)c3)cccc21]

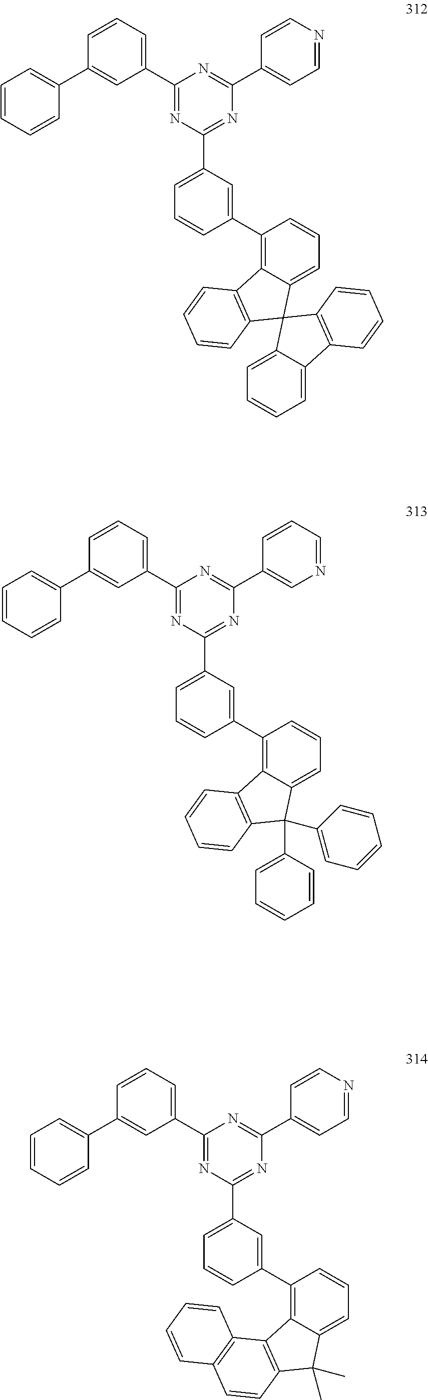 [SMILES: c1ccc(-c2cccc(-c3nc(-c4ccncc4)nc(-c4cccc(-c5cccc6c5-c5ccccc5C65c6ccccc6-c6ccccc65)c4)n3)c2)cc1 ; c1ccc(-c2cccc(-c3nc(-c4cccnc4)nc(-c4cccc(-c5cccc6c5-c5ccccc5C6(c5ccccc5)c5ccccc5)c4)n3)c2)cc1 ; CC1(C)c2cccc(-c3cccc(-c4nc(-c5ccncc5)nc(-c5cccc(-c6ccccc6)c5)n4)c3)c2-c2c1ccc1ccccc21]

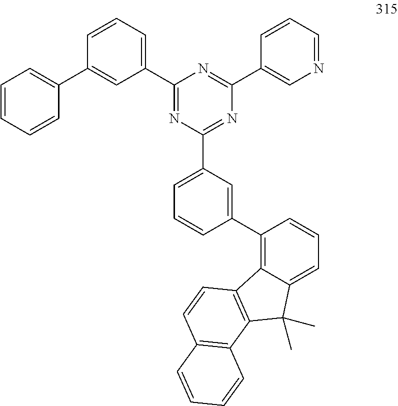 [SMILES: CC1(C)c2cccc(-c3cccc(-c4nc(-c5cccnc5)nc(-c5cccc(-c6ccccc6)c5)n4)c3)c2-c2ccc3ccccc3c21]

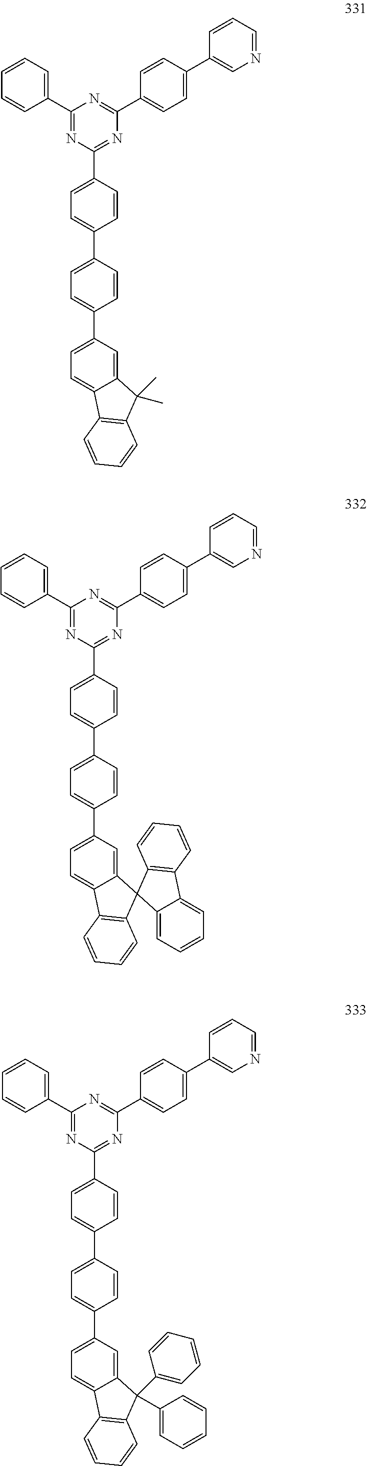 [SMILES: CC1(C)c2ccccc2-c2ccc(-c3ccc(-c4ccc(-c5nc(-c6ccccc6)nc(-c6ccc(-c7cccnc7)cc6)n5)cc4)cc3)cc21 ; c1ccc(-c2nc(-c3ccc(-c4ccc(-c5ccc6c(c5)C5(c7ccccc7-c7ccccc75)c5ccccc5-6)cc4)cc3)nc(-c3ccc(-c4cccnc4)cc3)n2)cc1 ; c1ccc(-c2nc(-c3ccc(-c4ccc(-c5ccc6c(c5)C(c5ccccc5)(c5ccccc5)c5ccccc5-6)cc4)cc3)nc(-c3ccc(-c4cccnc4)cc3)n2)cc1]

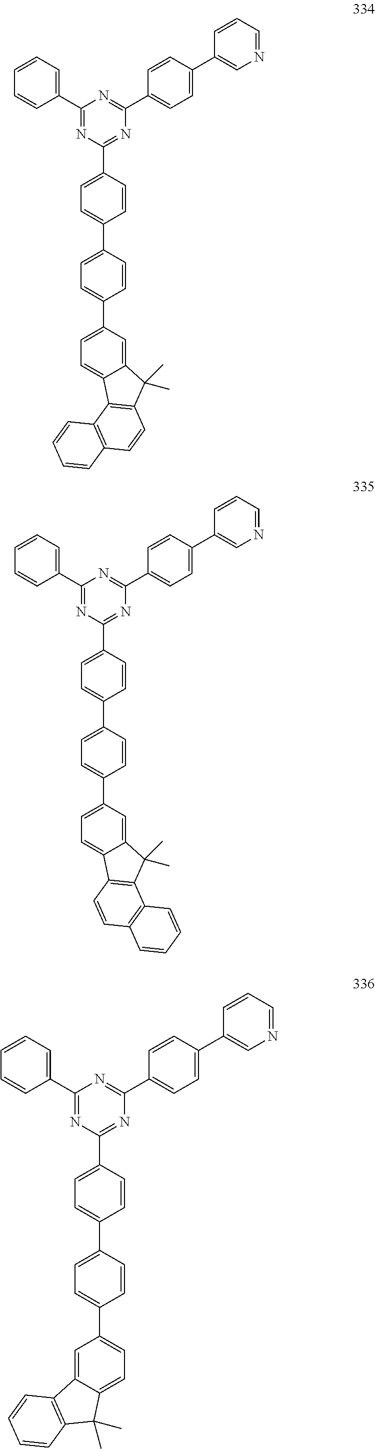 [SMILES: CC1(C)c2cc(-c3ccc(-c4ccc(-c5nc(-c6ccccc6)nc(-c6ccc(-c7cccnc7)cc6)n5)cc4)cc3)ccc2-c2c1ccc1ccccc21 ; CC1(C)c2cc(-c3ccc(-c4ccc(-c5nc(-c6ccccc6)nc(-c6ccc(-c7cccnc7)cc6)n5)cc4)cc3)ccc2-c2ccc3ccccc3c21 ; CC1(C)c2ccccc2-c2cc(-c3ccc(-c4ccc(-c5nc(-c6ccccc6)nc(-c6ccc(-c7cccnc7)cc6)n5)cc4)cc3)ccc21]

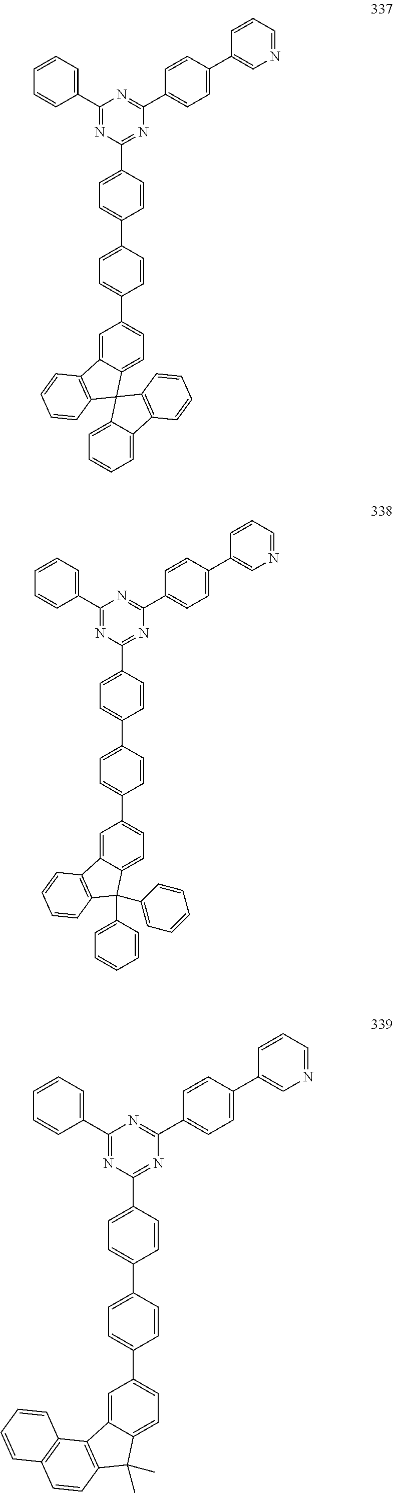 [SMILES: c1ccc(-c2nc(-c3ccc(-c4ccc(-c5ccc6c(c5)-c5ccccc5C65c6ccccc6-c6ccccc65)cc4)cc3)nc(-c3ccc(-c4cccnc4)cc3)n2)cc1 ; c1ccc(-c2nc(-c3ccc(-c4ccc(-c5ccc6c(c5)-c5ccccc5C6(c5ccccc5)c5ccccc5)cc4)cc3)nc(-c3ccc(-c4cccnc4)cc3)n2)cc1 ; CC1(C)c2ccc(-c3ccc(-c4ccc(-c5nc(-c6ccccc6)nc(-c6ccc(-c7cccnc7)cc6)n5)cc4)cc3)cc2-c2c1ccc1ccccc21]

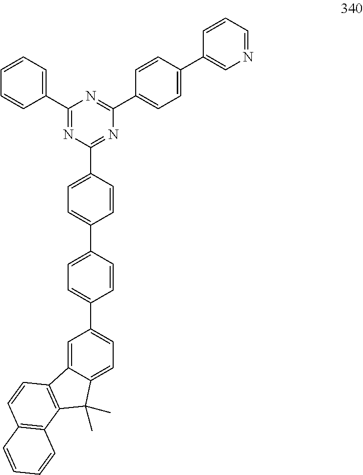 [SMILES: CC1(C)c2ccc(-c3ccc(-c4ccc(-c5nc(-c6ccccc6)nc(-c6ccc(-c7cccnc7)cc6)n5)cc4)cc3)cc2-c2ccc3ccccc3c21]

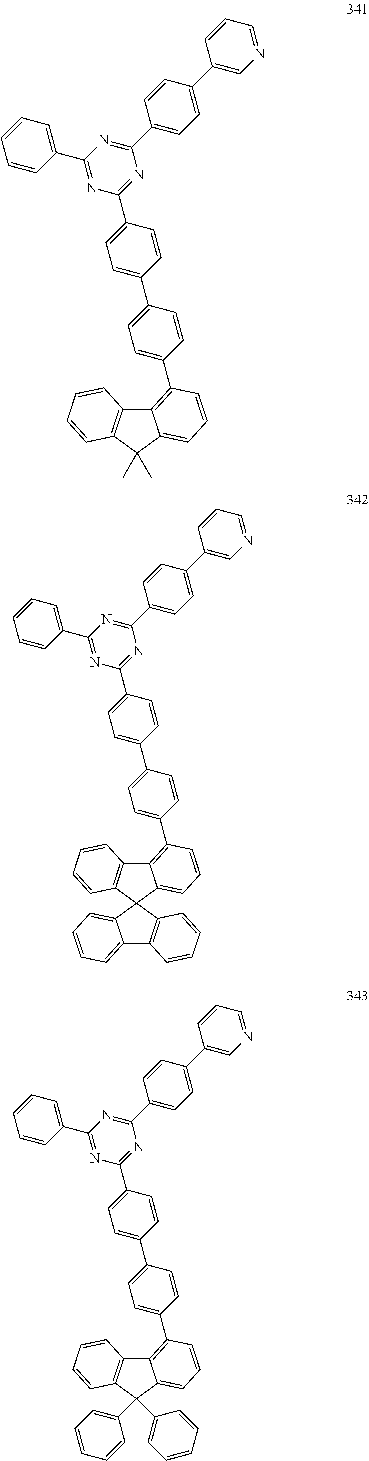 [SMILES: CC1(C)c2ccccc2-c2c(-c3ccc(-c4ccc(-c5nc(-c6ccccc6)nc(-c6ccc(-c7cccnc7)cc6)n5)cc4)cc3)cccc21 ; c1ccc(-c2nc(-c3ccc(-c4ccc(-c5cccc6c5-c5ccccc5C65c6ccccc6-c6ccccc65)cc4)cc3)nc(-c3ccc(-c4cccnc4)cc3)n2)cc1 ; c1ccc(-c2nc(-c3ccc(-c4ccc(-c5cccc6c5-c5ccccc5C6(c5ccccc5)c5ccccc5)cc4)cc3)nc(-c3ccc(-c4cccnc4)cc3)n2)cc1]

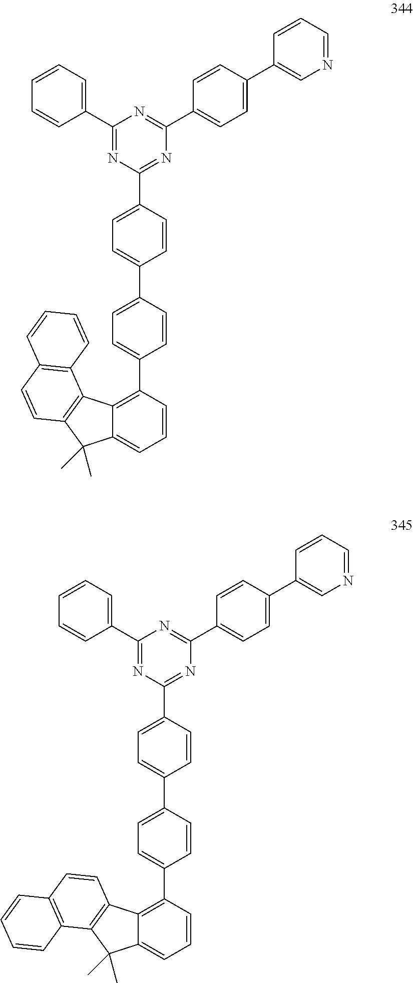 [SMILES: CC1(C)c2cccc(-c3ccc(-c4ccc(-c5nc(-c6ccccc6)nc(-c6ccc(-c7cccnc7)cc6)n5)cc4)cc3)c2-c2c1ccc1ccccc21 ; CC1(C)c2cccc(-c3ccc(-c4ccc(-c5nc(-c6ccccc6)nc(-c6ccc(-c7cccnc7)cc6)n5)cc4)cc3)c2-c2ccc3ccccc3c21]

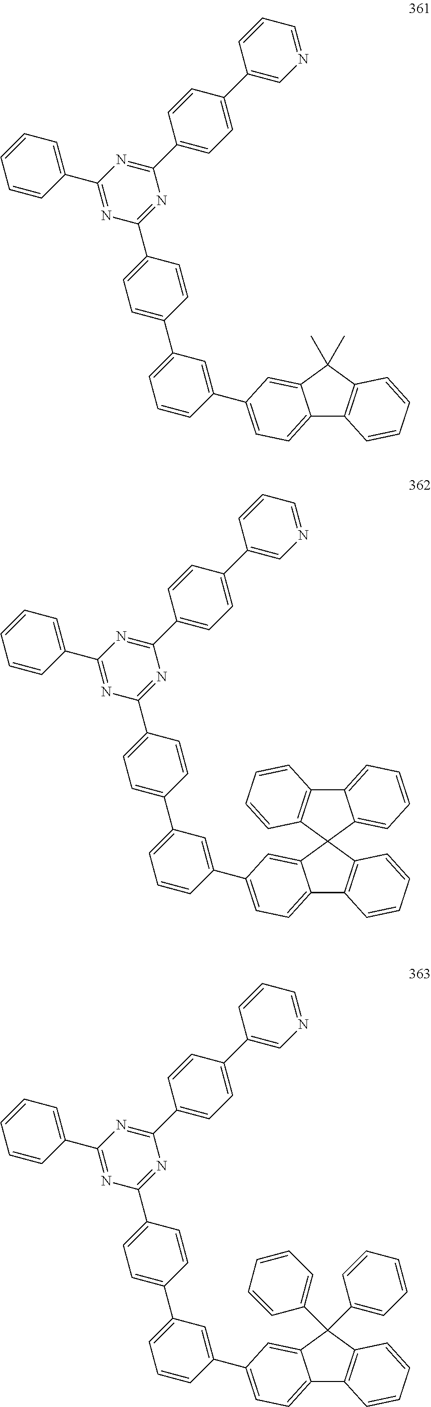 [SMILES: CC1(C)c2ccccc2-c2ccc(-c3cccc(-c4ccc(-c5nc(-c6ccccc6)nc(-c6ccc(-c7cccnc7)cc6)n5)cc4)c3)cc21 ; c1ccc(-c2nc(-c3ccc(-c4cccnc4)cc3)nc(-c3ccc(-c4cccc(-c5ccc6c(c5)C5(c7ccccc7-c7ccccc75)c5ccccc5-6)c4)cc3)n2)cc1 ; c1ccc(-c2nc(-c3ccc(-c4cccnc4)cc3)nc(-c3ccc(-c4cccc(-c5ccc6c(c5)C(c5ccccc5)(c5ccccc5)c5ccccc5-6)c4)cc3)n2)cc1]

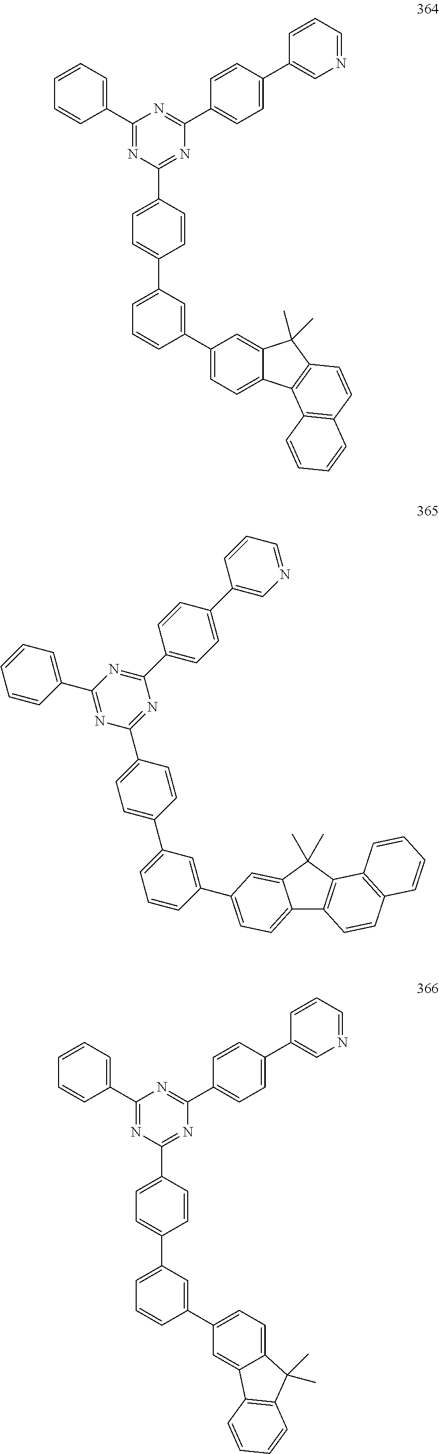 [SMILES: CC1(C)c2cc(-c3cccc(-c4ccc(-c5nc(-c6ccccc6)nc(-c6ccc(-c7cccnc7)cc6)n5)cc4)c3)ccc2-c2c1ccc1ccccc21 ; CC1(C)c2cc(-c3cccc(-c4ccc(-c5nc(-c6ccccc6)nc(-c6ccc(-c7cccnc7)cc6)n5)cc4)c3)ccc2-c2ccc3ccccc3c21 ; CC1(C)c2ccccc2-c2cc(-c3cccc(-c4ccc(-c5nc(-c6ccccc6)nc(-c6ccc(-c7cccnc7)cc6)n5)cc4)c3)ccc21]

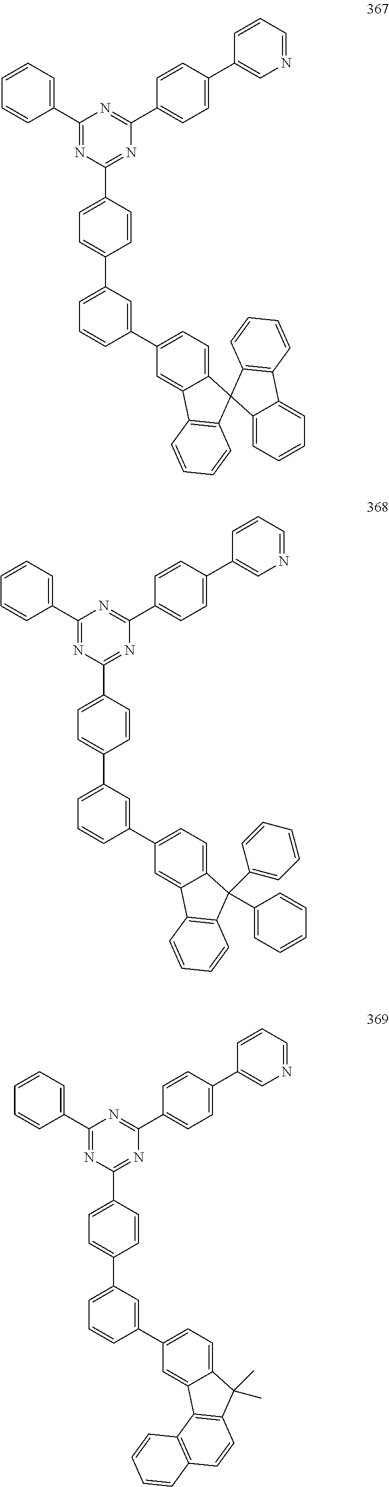 [SMILES: c1ccc(-c2nc(-c3ccc(-c4cccnc4)cc3)nc(-c3ccc(-c4cccc(-c5ccc6c(c5)-c5ccccc5C65c6ccccc6-c6ccccc65)c4)cc3)n2)cc1 ; c1ccc(-c2nc(-c3ccc(-c4cccnc4)cc3)nc(-c3ccc(-c4cccc(-c5ccc6c(c5)-c5ccccc5C6(c5ccccc5)c5ccccc5)c4)cc3)n2)cc1 ; CC1(C)c2ccc(-c3cccc(-c4ccc(-c5nc(-c6ccccc6)nc(-c6ccc(-c7cccnc7)cc6)n5)cc4)c3)cc2-c2c1ccc1ccccc21]

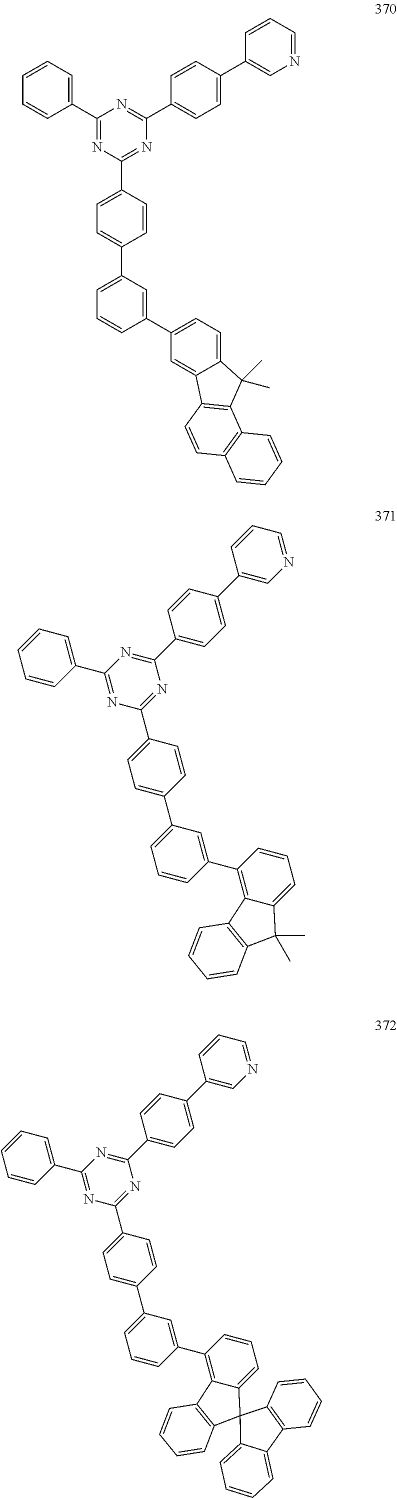 [SMILES: CC1(C)c2ccc(-c3cccc(-c4ccc(-c5nc(-c6ccccc6)nc(-c6ccc(-c7cccnc7)cc6)n5)cc4)c3)cc2-c2ccc3ccccc3c21 ; CC1(C)c2ccccc2-c2c(-c3cccc(-c4ccc(-c5nc(-c6ccccc6)nc(-c6ccc(-c7cccnc7)cc6)n5)cc4)c3)cccc21 ; c1ccc(-c2nc(-c3ccc(-c4cccnc4)cc3)nc(-c3ccc(-c4cccc(-c5cccc6c5-c5ccccc5C65c6ccccc6-c6ccccc65)c4)cc3)n2)cc1]

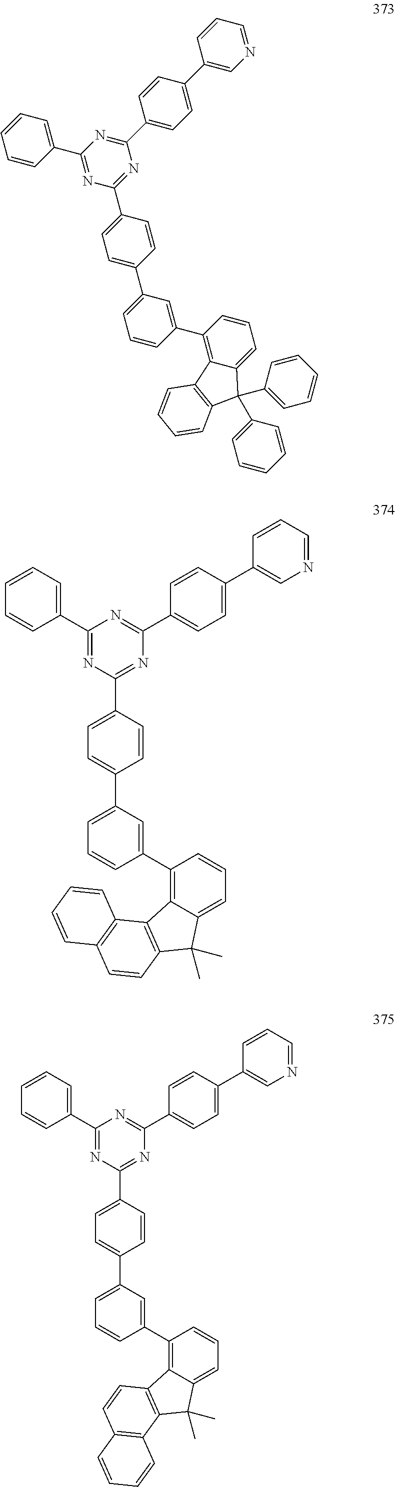 [SMILES: c1ccc(-c2nc(-c3ccc(-c4cccnc4)cc3)nc(-c3ccc(-c4cccc(-c5cccc6c5-c5ccccc5C6(c5ccccc5)c5ccccc5)c4)cc3)n2)cc1 ; CC1(C)c2cccc(-c3cccc(-c4ccc(-c5nc(-c6ccccc6)nc(-c6ccc(-c7cccnc7)cc6)n5)cc4)c3)c2-c2c1ccc1ccccc21 ; CC1(C)c2cccc(-c3cccc(-c4ccc(-c5nc(-c6ccccc6)nc(-c6ccc(-c7cccnc7)cc6)n5)cc4)c3)c2-c2ccc3ccccc3c21]

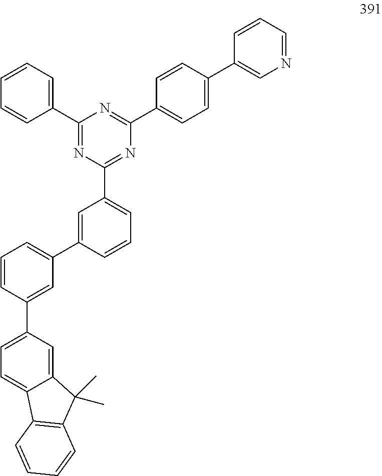 [SMILES: CC1(C)c2ccccc2-c2ccc(-c3cccc(-c4cccc(-c5nc(-c6ccccc6)nc(-c6ccc(-c7cccnc7)cc6)n5)c4)c3)cc21]

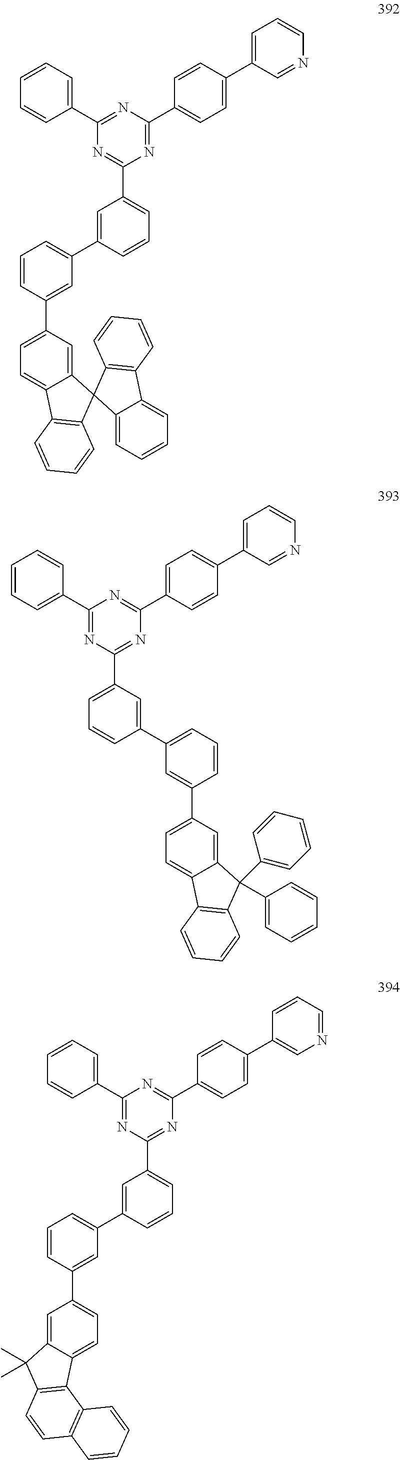 [SMILES: c1ccc(-c2nc(-c3ccc(-c4cccnc4)cc3)nc(-c3cccc(-c4cccc(-c5ccc6c(c5)C5(c7ccccc7-c7ccccc75)c5ccccc5-6)c4)c3)n2)cc1 ; c1ccc(-c2nc(-c3ccc(-c4cccnc4)cc3)nc(-c3cccc(-c4cccc(-c5ccc6c(c5)C(c5ccccc5)(c5ccccc5)c5ccccc5-6)c4)c3)n2)cc1 ; CC1(C)c2cc(-c3cccc(-c4cccc(-c5nc(-c6ccccc6)nc(-c6ccc(-c7cccnc7)cc6)n5)c4)c3)ccc2-c2c1ccc1ccccc21]

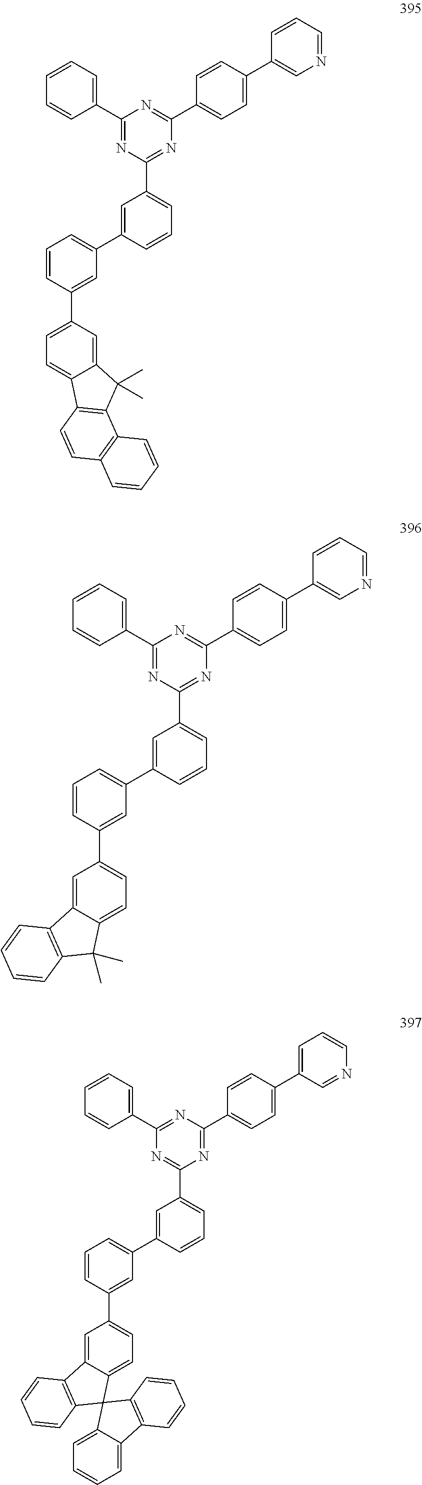 [SMILES: CC1(C)c2cc(-c3cccc(-c4cccc(-c5nc(-c6ccccc6)nc(-c6ccc(-c7cccnc7)cc6)n5)c4)c3)ccc2-c2ccc3ccccc3c21 ; CC1(C)c2ccccc2-c2cc(-c3cccc(-c4cccc(-c5nc(-c6ccccc6)nc(-c6ccc(-c7cccnc7)cc6)n5)c4)c3)ccc21 ; c1ccc(-c2nc(-c3ccc(-c4cccnc4)cc3)nc(-c3cccc(-c4cccc(-c5ccc6c(c5)-c5ccccc5C65c6ccccc6-c6ccccc65)c4)c3)n2)cc1]

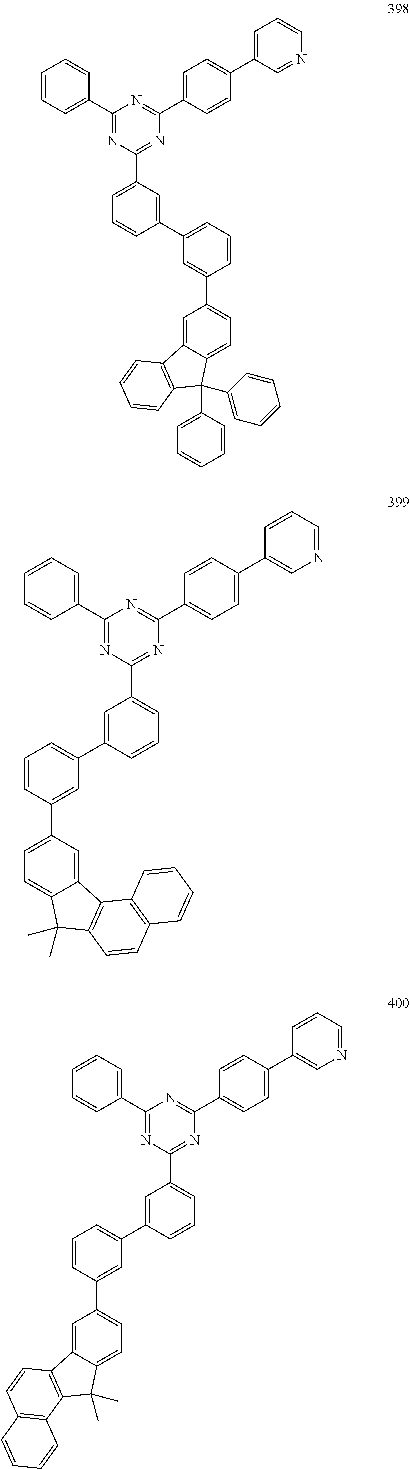 [SMILES: c1ccc(-c2nc(-c3ccc(-c4cccnc4)cc3)nc(-c3cccc(-c4cccc(-c5ccc6c(c5)-c5ccccc5C6(c5ccccc5)c5ccccc5)c4)c3)n2)cc1 ; CC1(C)c2ccc(-c3cccc(-c4cccc(-c5nc(-c6ccccc6)nc(-c6ccc(-c7cccnc7)cc6)n5)c4)c3)cc2-c2c1ccc1ccccc21 ; CC1(C)c2ccc(-c3cccc(-c4cccc(-c5nc(-c6ccccc6)nc(-c6ccc(-c7cccnc7)cc6)n5)c4)c3)cc2-c2ccc3ccccc3c21]

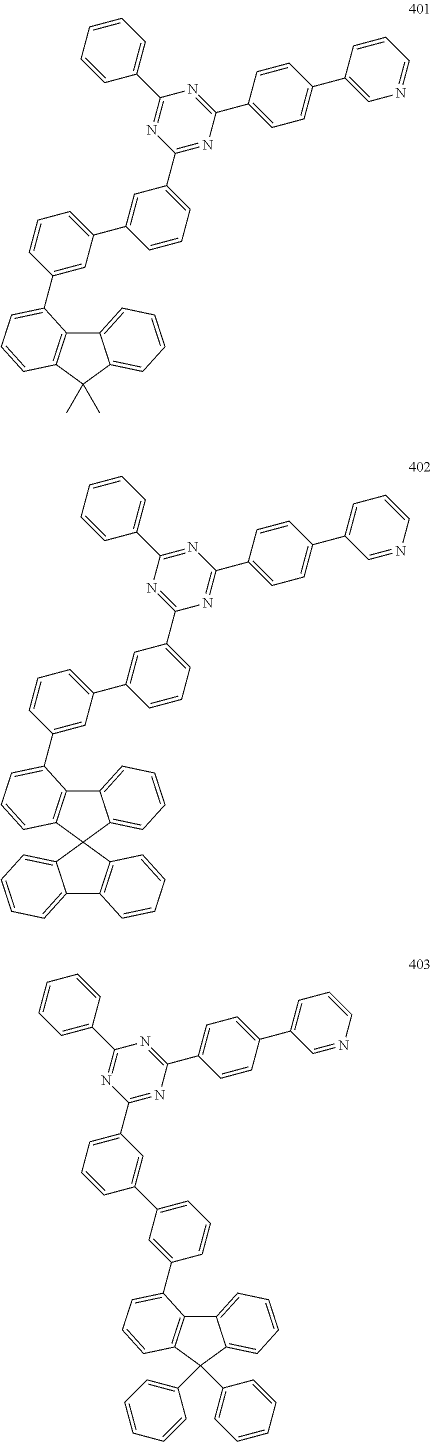 [SMILES: CC1(C)c2ccccc2-c2c(-c3cccc(-c4cccc(-c5nc(-c6ccccc6)nc(-c6ccc(-c7cccnc7)cc6)n5)c4)c3)cccc21 ; c1ccc(-c2nc(-c3ccc(-c4cccnc4)cc3)nc(-c3cccc(-c4cccc(-c5cccc6c5-c5ccccc5C65c6ccccc6-c6ccccc65)c4)c3)n2)cc1 ; c1ccc(-c2nc(-c3ccc(-c4cccnc4)cc3)nc(-c3cccc(-c4cccc(-c5cccc6c5-c5ccccc5C6(c5ccccc5)c5ccccc5)c4)c3)n2)cc1]

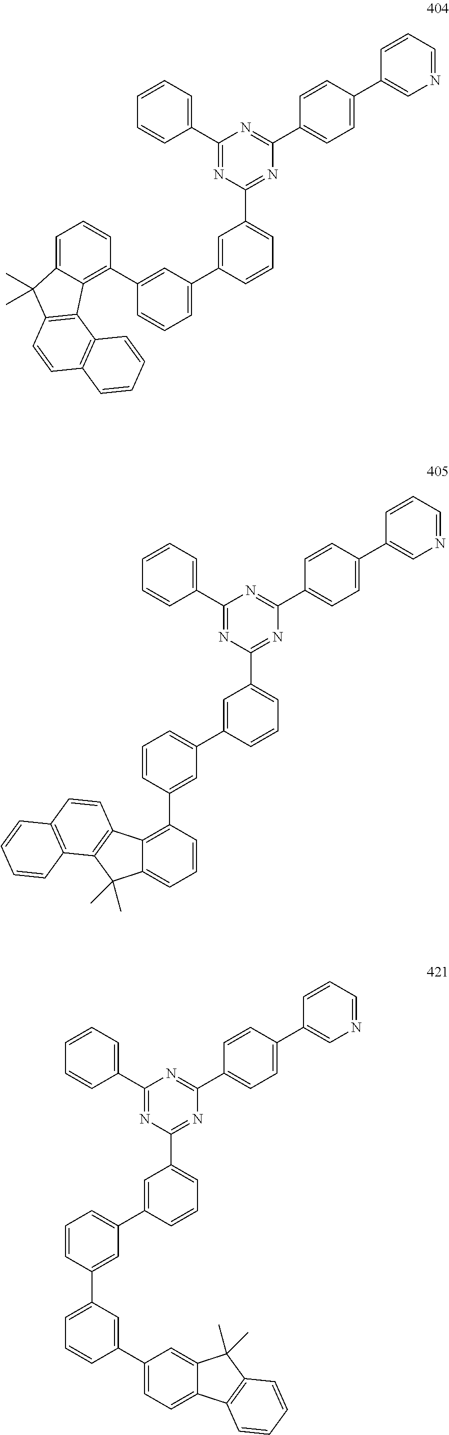 [SMILES: CC1(C)c2cccc(-c3cccc(-c4cccc(-c5nc(-c6ccccc6)nc(-c6ccc(-c7cccnc7)cc6)n5)c4)c3)c2-c2c1ccc1ccccc21 ; CC1(C)c2cccc(-c3cccc(-c4cccc(-c5nc(-c6ccccc6)nc(-c6ccc(-c7cccnc7)cc6)n5)c4)c3)c2-c2ccc3ccccc3c21 ; CC1(C)c2ccccc2-c2ccc(-c3cccc(-c4cccc(-c5cccc(-c6nc(-c7ccccc7)nc(-c7ccc(-c8cccnc8)cc7)n6)c5)c4)c3)cc21]

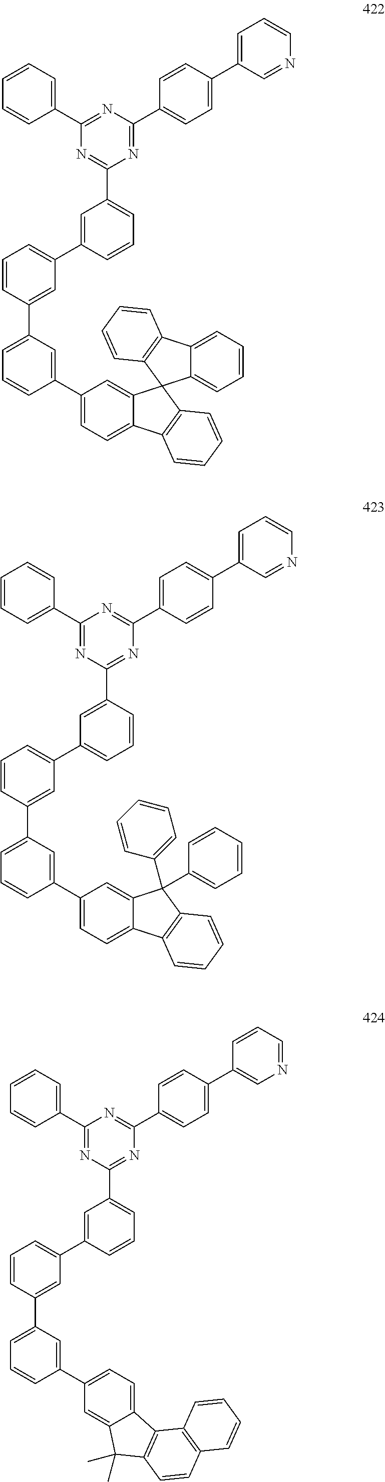 [SMILES: c1ccc(-c2nc(-c3ccc(-c4cccnc4)cc3)nc(-c3cccc(-c4cccc(-c5cccc(-c6ccc7c(c6)C6(c8ccccc8-c8ccccc86)c6ccccc6-7)c5)c4)c3)n2)cc1 ; c1ccc(-c2nc(-c3ccc(-c4cccnc4)cc3)nc(-c3cccc(-c4cccc(-c5cccc(-c6ccc7c(c6)C(c6ccccc6)(c6ccccc6)c6ccccc6-7)c5)c4)c3)n2)cc1 ; CC1(C)c2cc(-c3cccc(-c4cccc(-c5cccc(-c6nc(-c7ccccc7)nc(-c7ccc(-c8cccnc8)cc7)n6)c5)c4)c3)ccc2-c2c1ccc1ccccc21]

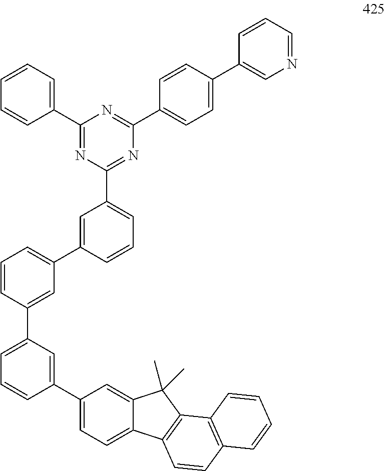 [SMILES: CC1(C)c2cc(-c3cccc(-c4cccc(-c5cccc(-c6nc(-c7ccccc7)nc(-c7ccc(-c8cccnc8)cc7)n6)c5)c4)c3)ccc2-c2ccc3ccccc3c21]

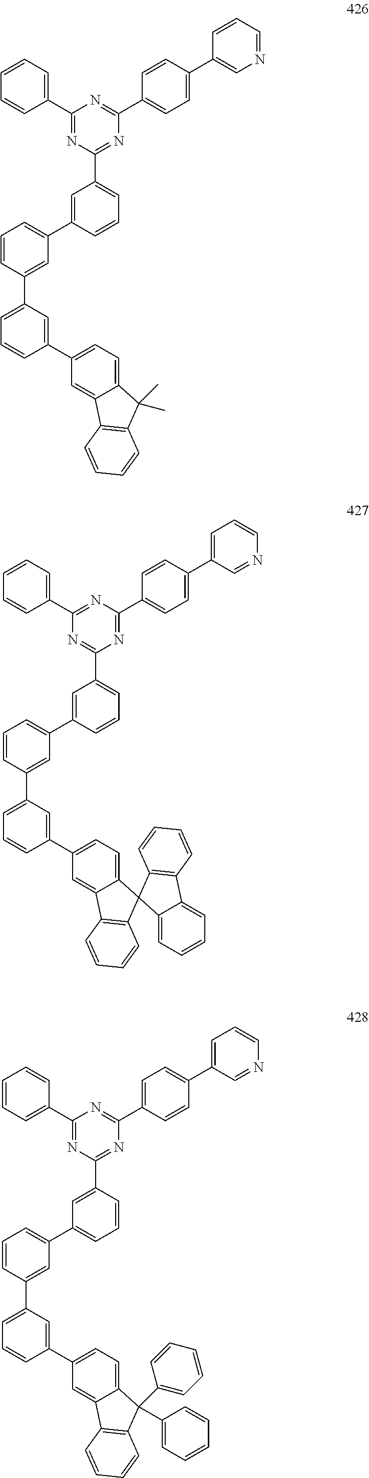 [SMILES: CC1(C)c2ccccc2-c2cc(-c3cccc(-c4cccc(-c5cccc(-c6nc(-c7ccccc7)nc(-c7ccc(-c8cccnc8)cc7)n6)c5)c4)c3)ccc21 ; c1ccc(-c2nc(-c3ccc(-c4cccnc4)cc3)nc(-c3cccc(-c4cccc(-c5cccc(-c6ccc7c(c6)-c6ccccc6C76c7ccccc7-c7ccccc76)c5)c4)c3)n2)cc1 ; c1ccc(-c2nc(-c3ccc(-c4cccnc4)cc3)nc(-c3cccc(-c4cccc(-c5cccc(-c6ccc7c(c6)-c6ccccc6C7(c6ccccc6)c6ccccc6)c5)c4)c3)n2)cc1]

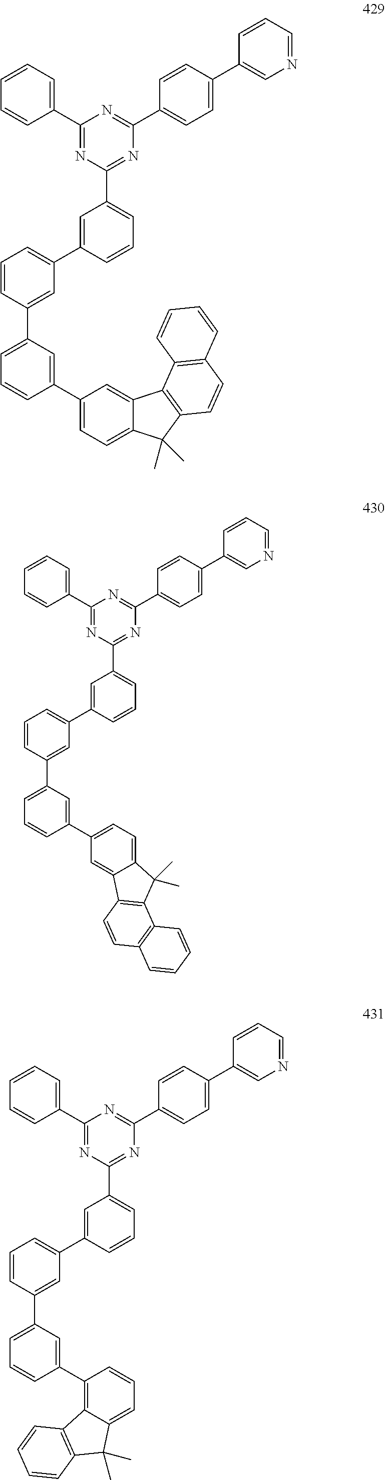 [SMILES: CC1(C)c2ccc(-c3cccc(-c4cccc(-c5cccc(-c6nc(-c7ccccc7)nc(-c7ccc(-c8cccnc8)cc7)n6)c5)c4)c3)cc2-c2c1ccc1ccccc21 ; CC1(C)c2ccc(-c3cccc(-c4cccc(-c5cccc(-c6nc(-c7ccccc7)nc(-c7ccc(-c8cccnc8)cc7)n6)c5)c4)c3)cc2-c2ccc3ccccc3c21 ; CC1(C)c2ccccc2-c2c(-c3cccc(-c4cccc(-c5cccc(-c6nc(-c7ccccc7)nc(-c7ccc(-c8cccnc8)cc7)n6)c5)c4)c3)cccc21]

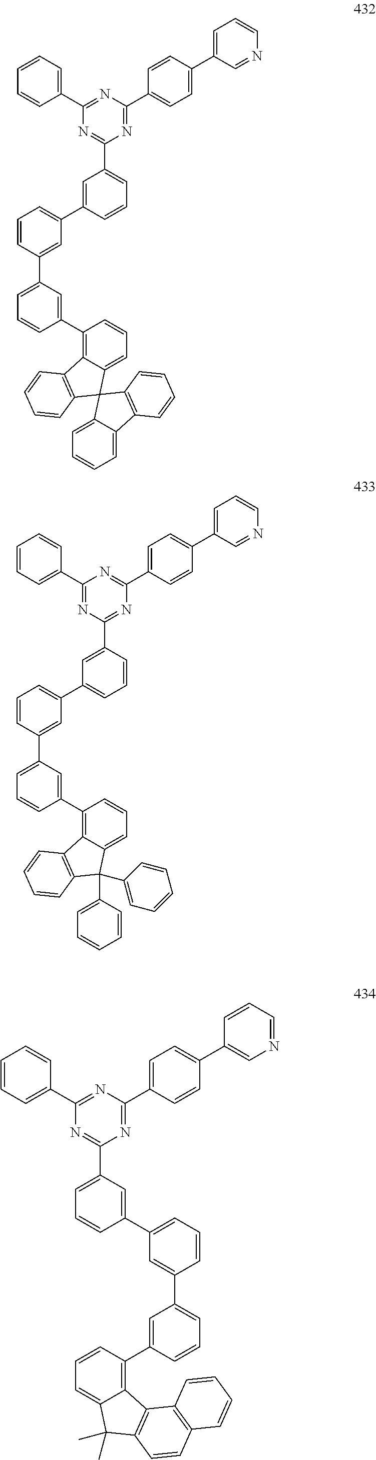 [SMILES: c1ccc(-c2nc(-c3ccc(-c4cccnc4)cc3)nc(-c3cccc(-c4cccc(-c5cccc(-c6cccc7c6-c6ccccc6C76c7ccccc7-c7ccccc76)c5)c4)c3)n2)cc1 ; c1ccc(-c2nc(-c3ccc(-c4cccnc4)cc3)nc(-c3cccc(-c4cccc(-c5cccc(-c6cccc7c6-c6ccccc6C7(c6ccccc6)c6ccccc6)c5)c4)c3)n2)cc1 ; CC1(C)c2cccc(-c3cccc(-c4cccc(-c5cccc(-c6nc(-c7ccccc7)nc(-c7ccc(-c8cccnc8)cc7)n6)c5)c4)c3)c2-c2c1ccc1ccccc21]

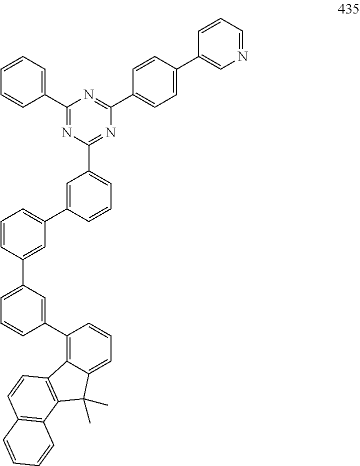 [SMILES: CC1(C)c2cccc(-c3cccc(-c4cccc(-c5cccc(-c6nc(-c7ccccc7)nc(-c7ccc(-c8cccnc8)cc7)n6)c5)c4)c3)c2-c2ccc3ccccc3c21]

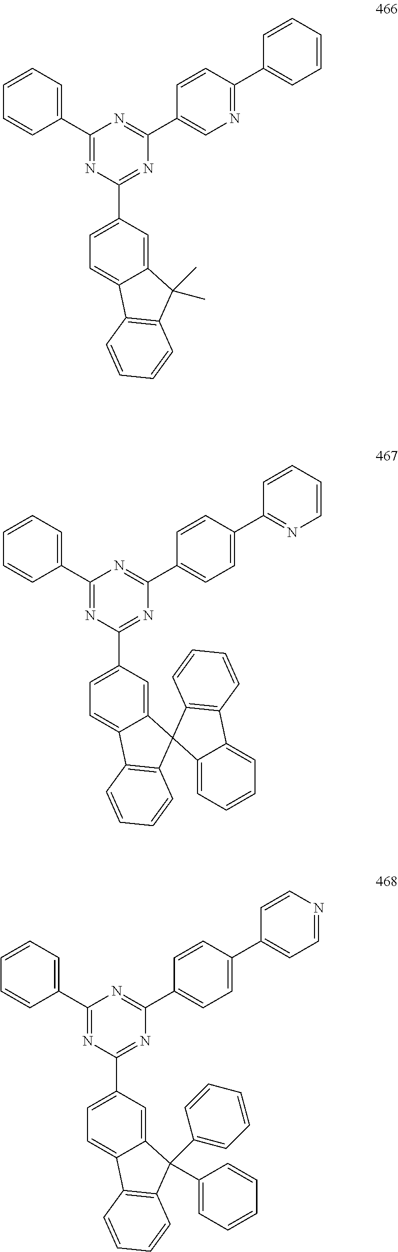 [SMILES: CC1(C)c2ccccc2-c2ccc(-c3nc(-c4ccccc4)nc(-c4ccc(-c5ccccc5)nc4)n3)cc21 ; c1ccc(-c2nc(-c3ccc(-c4ccccn4)cc3)nc(-c3ccc4c(c3)C3(c5ccccc5-c5ccccc53)c3ccccc3-4)n2)cc1 ; c1ccc(-c2nc(-c3ccc(-c4ccncc4)cc3)nc(-c3ccc4c(c3)C(c3ccccc3)(c3ccccc3)c3ccccc3-4)n2)cc1]

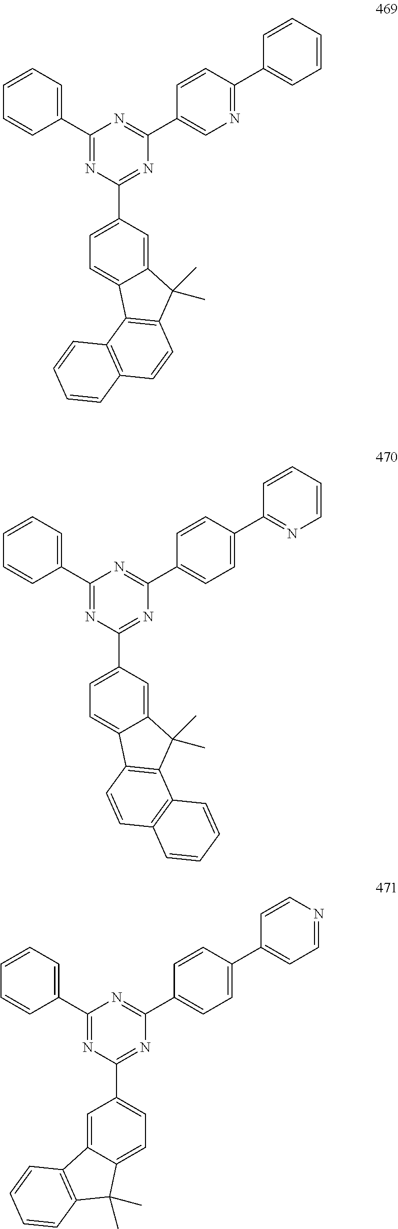 [SMILES: CC1(C)c2cc(-c3nc(-c4ccccc4)nc(-c4ccc(-c5ccccc5)nc4)n3)ccc2-c2c1ccc1ccccc21 ; CC1(C)c2cc(-c3nc(-c4ccccc4)nc(-c4ccc(-c5ccccn5)cc4)n3)ccc2-c2ccc3ccccc3c21 ; CC1(C)c2ccccc2-c2cc(-c3nc(-c4ccccc4)nc(-c4ccc(-c5ccncc5)cc4)n3)ccc21]

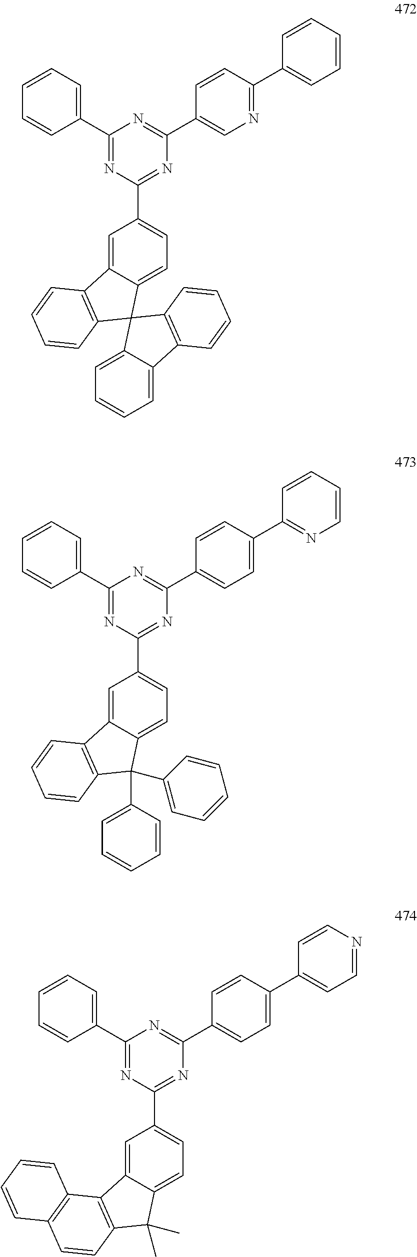 [SMILES: c1ccc(-c2ccc(-c3nc(-c4ccccc4)nc(-c4ccc5c(c4)-c4ccccc4C54c5ccccc5-c5ccccc54)n3)cn2)cc1 ; c1ccc(-c2nc(-c3ccc(-c4ccccn4)cc3)nc(-c3ccc4c(c3)-c3ccccc3C4(c3ccccc3)c3ccccc3)n2)cc1 ; CC1(C)c2ccc(-c3nc(-c4ccccc4)nc(-c4ccc(-c5ccncc5)cc4)n3)cc2-c2c1ccc1ccccc21]

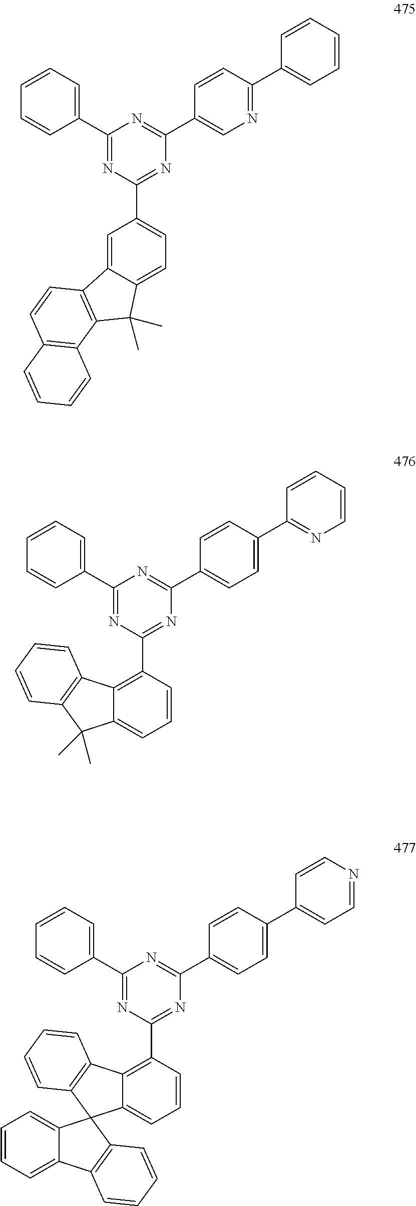 [SMILES: CC1(C)c2ccc(-c3nc(-c4ccccc4)nc(-c4ccc(-c5ccccc5)nc4)n3)cc2-c2ccc3ccccc3c21 ; CC1(C)c2ccccc2-c2c(-c3nc(-c4ccccc4)nc(-c4ccc(-c5ccccn5)cc4)n3)cccc21 ; c1ccc(-c2nc(-c3ccc(-c4ccncc4)cc3)nc(-c3cccc4c3-c3ccccc3C43c4ccccc4-c4ccccc43)n2)cc1]

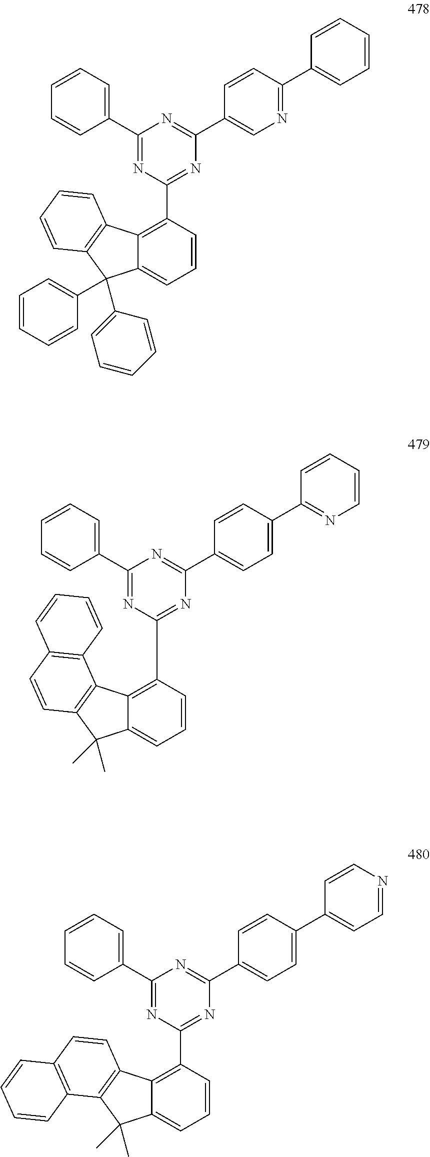 [SMILES: c1ccc(-c2ccc(-c3nc(-c4ccccc4)nc(-c4cccc5c4-c4ccccc4C5(c4ccccc4)c4ccccc4)n3)cn2)cc1 ; CC1(C)c2cccc(-c3nc(-c4ccccc4)nc(-c4ccc(-c5ccccn5)cc4)n3)c2-c2c1ccc1ccccc21 ; CC1(C)c2cccc(-c3nc(-c4ccccc4)nc(-c4ccc(-c5ccncc5)cc4)n3)c2-c2ccc3ccccc3c21]

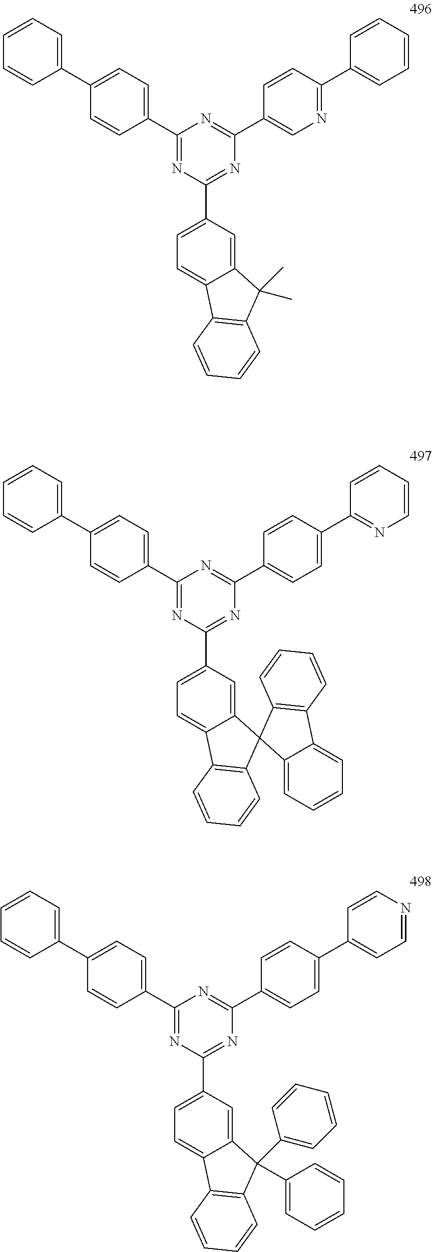 [SMILES: CC1(C)c2ccccc2-c2ccc(-c3nc(-c4ccc(-c5ccccc5)cc4)nc(-c4ccc(-c5ccccc5)nc4)n3)cc21 ; c1ccc(-c2ccc(-c3nc(-c4ccc(-c5ccccn5)cc4)nc(-c4ccc5c(c4)C4(c6ccccc6-c6ccccc64)c4ccccc4-5)n3)cc2)cc1 ; c1ccc(-c2ccc(-c3nc(-c4ccc(-c5ccncc5)cc4)nc(-c4ccc5c(c4)C(c4ccccc4)(c4ccccc4)c4ccccc4-5)n3)cc2)cc1]

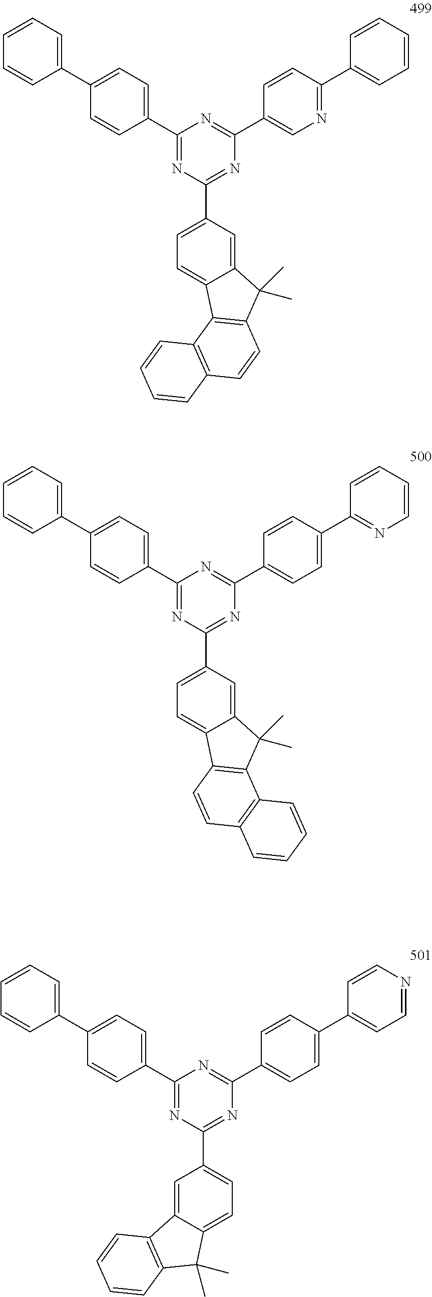 [SMILES: CC1(C)c2cc(-c3nc(-c4ccc(-c5ccccc5)cc4)nc(-c4ccc(-c5ccccc5)nc4)n3)ccc2-c2c1ccc1ccccc21 ; CC1(C)c2cc(-c3nc(-c4ccc(-c5ccccc5)cc4)nc(-c4ccc(-c5ccccn5)cc4)n3)ccc2-c2ccc3ccccc3c21 ; CC1(C)c2ccccc2-c2cc(-c3nc(-c4ccc(-c5ccccc5)cc4)nc(-c4ccc(-c5ccncc5)cc4)n3)ccc21]

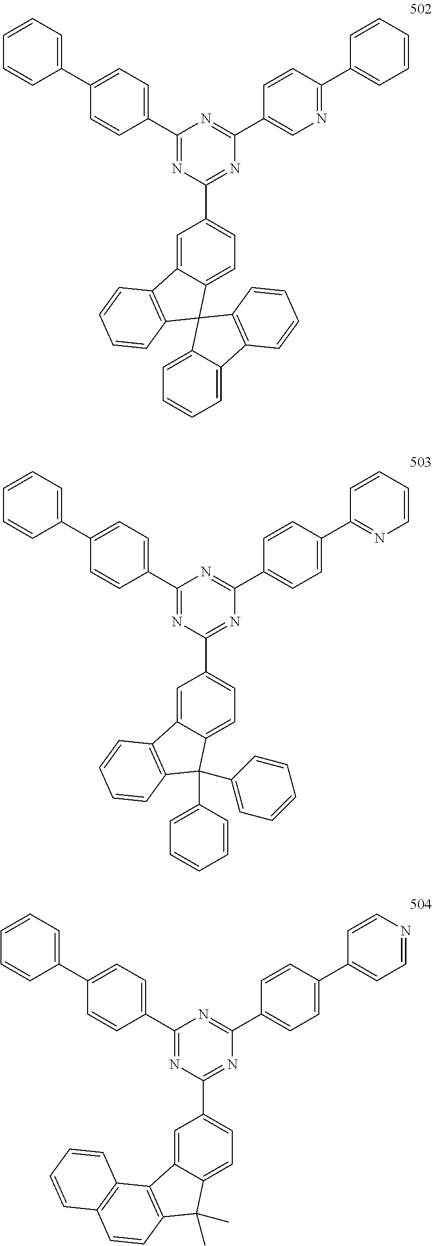 [SMILES: c1ccc(-c2ccc(-c3nc(-c4ccc(-c5ccccc5)nc4)nc(-c4ccc5c(c4)-c4ccccc4C54c5ccccc5-c5ccccc54)n3)cc2)cc1 ; c1ccc(-c2ccc(-c3nc(-c4ccc(-c5ccccn5)cc4)nc(-c4ccc5c(c4)-c4ccccc4C5(c4ccccc4)c4ccccc4)n3)cc2)cc1 ; CC1(C)c2ccc(-c3nc(-c4ccc(-c5ccccc5)cc4)nc(-c4ccc(-c5ccncc5)cc4)n3)cc2-c2c1ccc1ccccc21]

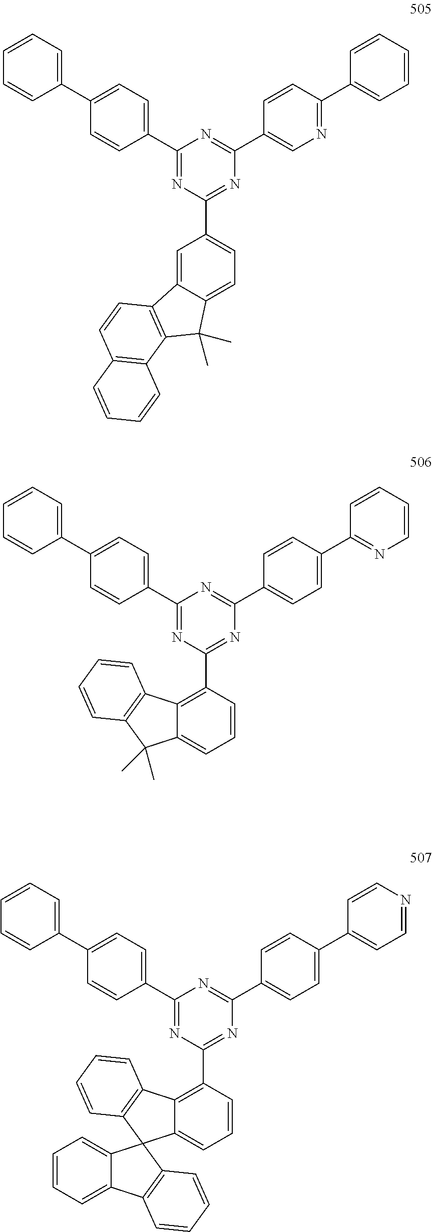 [SMILES: CC1(C)c2ccc(-c3nc(-c4ccc(-c5ccccc5)cc4)nc(-c4ccc(-c5ccccc5)nc4)n3)cc2-c2ccc3ccccc3c21 ; CC1(C)c2ccccc2-c2c(-c3nc(-c4ccc(-c5ccccc5)cc4)nc(-c4ccc(-c5ccccn5)cc4)n3)cccc21 ; c1ccc(-c2ccc(-c3nc(-c4ccc(-c5ccncc5)cc4)nc(-c4cccc5c4-c4ccccc4C54c5ccccc5-c5ccccc54)n3)cc2)cc1]

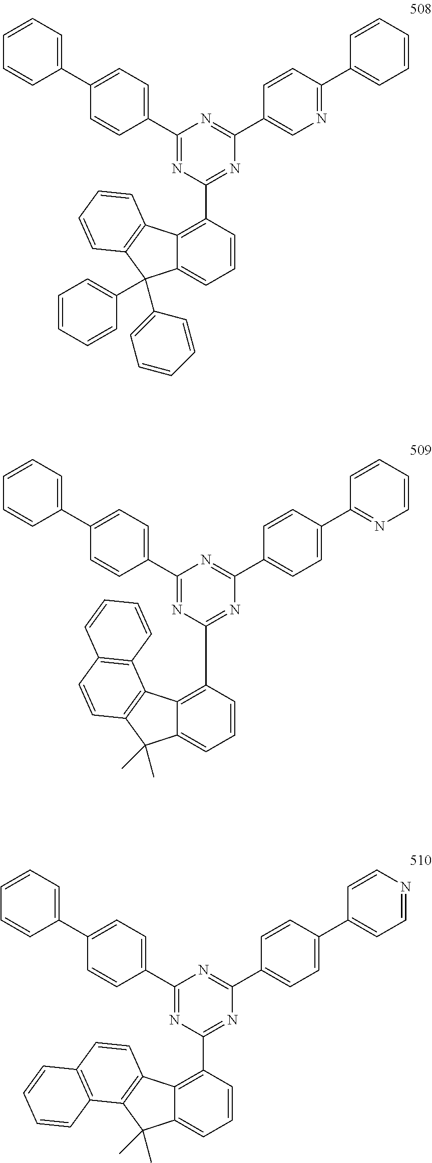 [SMILES: c1ccc(-c2ccc(-c3nc(-c4ccc(-c5ccccc5)nc4)nc(-c4cccc5c4-c4ccccc4C5(c4ccccc4)c4ccccc4)n3)cc2)cc1 ; CC1(C)c2cccc(-c3nc(-c4ccc(-c5ccccc5)cc4)nc(-c4ccc(-c5ccccn5)cc4)n3)c2-c2c1ccc1ccccc21 ; CC1(C)c2cccc(-c3nc(-c4ccc(-c5ccccc5)cc4)nc(-c4ccc(-c5ccncc5)cc4)n3)c2-c2ccc3ccccc3c21]

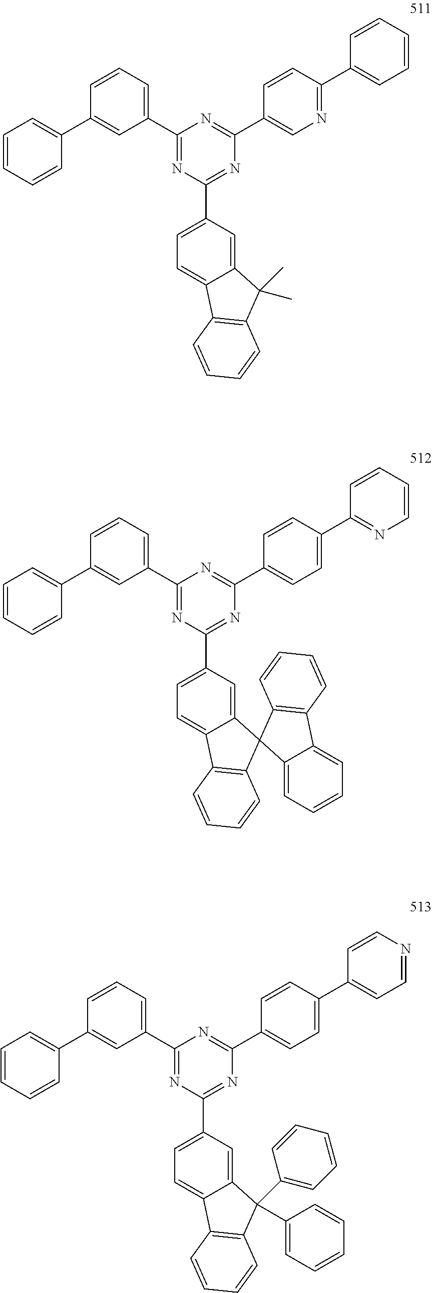 [SMILES: CC1(C)c2ccccc2-c2ccc(-c3nc(-c4ccc(-c5ccccc5)nc4)nc(-c4cccc(-c5ccccc5)c4)n3)cc21 ; c1ccc(-c2cccc(-c3nc(-c4ccc(-c5ccccn5)cc4)nc(-c4ccc5c(c4)C4(c6ccccc6-c6ccccc64)c4ccccc4-5)n3)c2)cc1 ; c1ccc(-c2cccc(-c3nc(-c4ccc(-c5ccncc5)cc4)nc(-c4ccc5c(c4)C(c4ccccc4)(c4ccccc4)c4ccccc4-5)n3)c2)cc1]

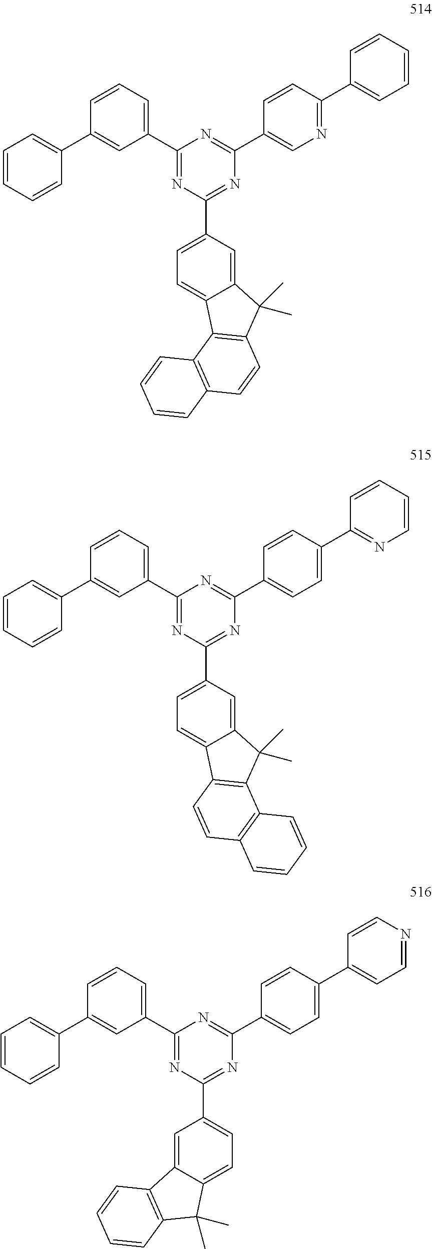 [SMILES: CC1(C)c2cc(-c3nc(-c4ccc(-c5ccccc5)nc4)nc(-c4cccc(-c5ccccc5)c4)n3)ccc2-c2c1ccc1ccccc21 ; CC1(C)c2cc(-c3nc(-c4ccc(-c5ccccn5)cc4)nc(-c4cccc(-c5ccccc5)c4)n3)ccc2-c2ccc3ccccc3c21 ; CC1(C)c2ccccc2-c2cc(-c3nc(-c4ccc(-c5ccncc5)cc4)nc(-c4cccc(-c5ccccc5)c4)n3)ccc21]

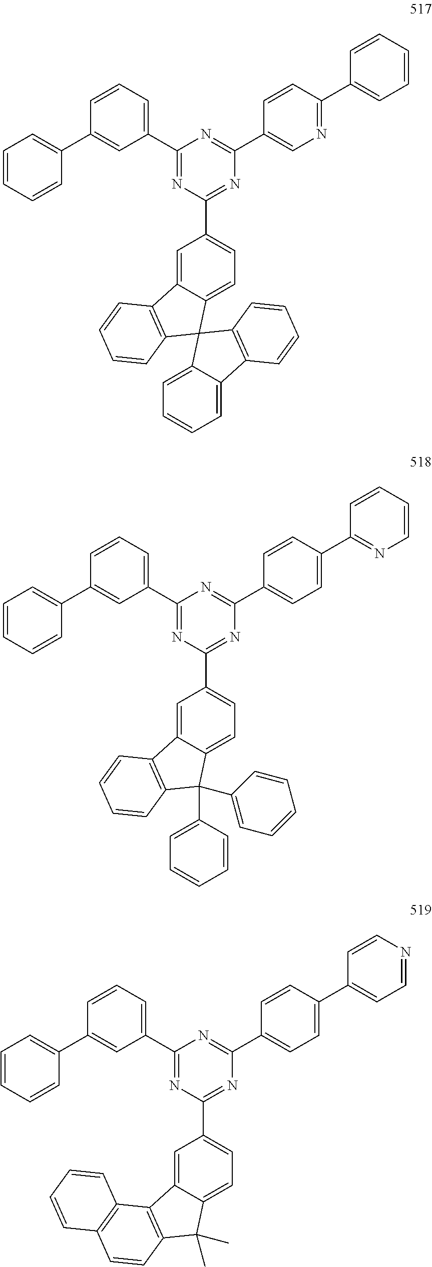 [SMILES: c1ccc(-c2cccc(-c3nc(-c4ccc(-c5ccccc5)nc4)nc(-c4ccc5c(c4)-c4ccccc4C54c5ccccc5-c5ccccc54)n3)c2)cc1 ; c1ccc(-c2cccc(-c3nc(-c4ccc(-c5ccccn5)cc4)nc(-c4ccc5c(c4)-c4ccccc4C5(c4ccccc4)c4ccccc4)n3)c2)cc1 ; CC1(C)c2ccc(-c3nc(-c4ccc(-c5ccncc5)cc4)nc(-c4cccc(-c5ccccc5)c4)n3)cc2-c2c1ccc1ccccc21]

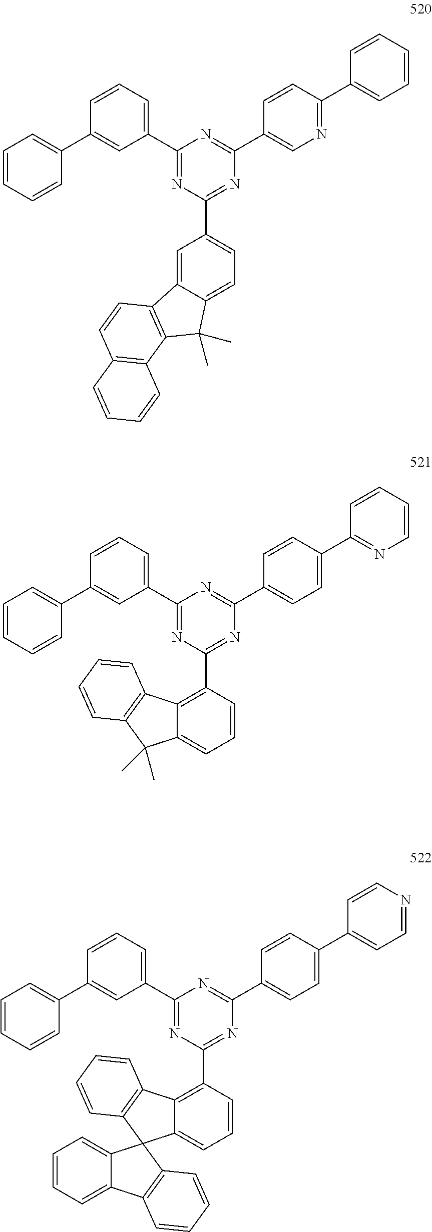 [SMILES: CC1(C)c2ccc(-c3nc(-c4ccc(-c5ccccc5)nc4)nc(-c4cccc(-c5ccccc5)c4)n3)cc2-c2ccc3ccccc3c21 ; CC1(C)c2ccccc2-c2c(-c3nc(-c4ccc(-c5ccccn5)cc4)nc(-c4cccc(-c5ccccc5)c4)n3)cccc21 ; c1ccc(-c2cccc(-c3nc(-c4ccc(-c5ccncc5)cc4)nc(-c4cccc5c4-c4ccccc4C54c5ccccc5-c5ccccc54)n3)c2)cc1]

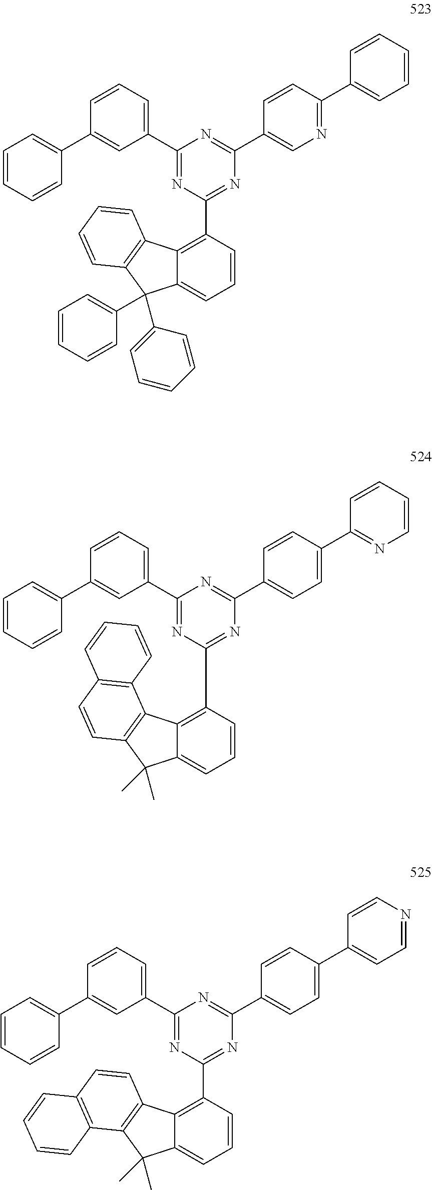 [SMILES: c1ccc(-c2cccc(-c3nc(-c4ccc(-c5ccccc5)nc4)nc(-c4cccc5c4-c4ccccc4C5(c4ccccc4)c4ccccc4)n3)c2)cc1 ; CC1(C)c2cccc(-c3nc(-c4ccc(-c5ccccn5)cc4)nc(-c4cccc(-c5ccccc5)c4)n3)c2-c2c1ccc1ccccc21 ; CC1(C)c2cccc(-c3nc(-c4ccc(-c5ccncc5)cc4)nc(-c4cccc(-c5ccccc5)c4)n3)c2-c2ccc3ccccc3c21]

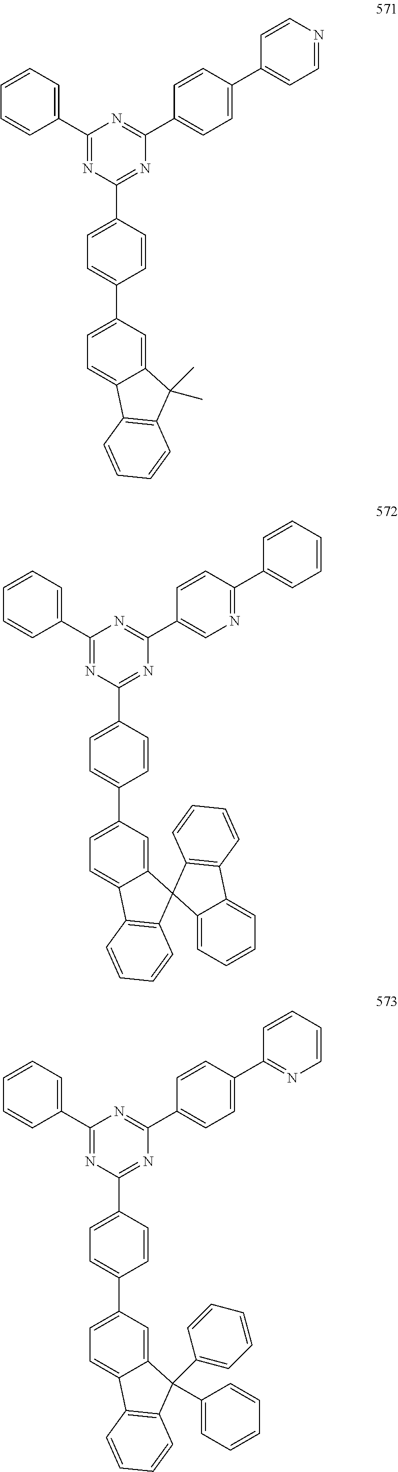 [SMILES: CC1(C)c2ccccc2-c2ccc(-c3ccc(-c4nc(-c5ccccc5)nc(-c5ccc(-c6ccncc6)cc5)n4)cc3)cc21 ; c1ccc(-c2ccc(-c3nc(-c4ccccc4)nc(-c4ccc(-c5ccc6c(c5)C5(c7ccccc7-c7ccccc75)c5ccccc5-6)cc4)n3)cn2)cc1 ; c1ccc(-c2nc(-c3ccc(-c4ccc5c(c4)C(c4ccccc4)(c4ccccc4)c4ccccc4-5)cc3)nc(-c3ccc(-c4ccccn4)cc3)n2)cc1]

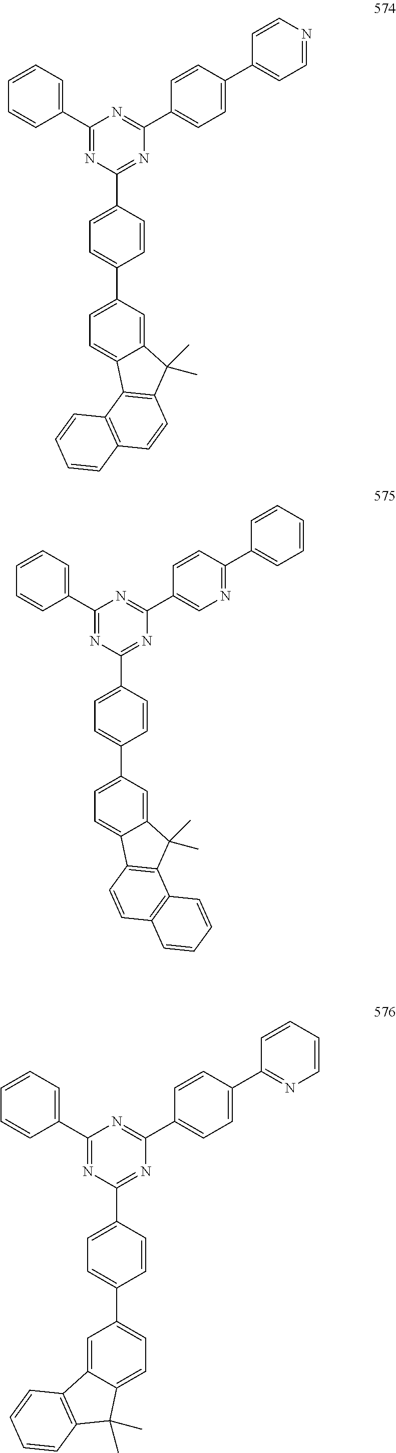 [SMILES: CC1(C)c2cc(-c3ccc(-c4nc(-c5ccccc5)nc(-c5ccc(-c6ccncc6)cc5)n4)cc3)ccc2-c2c1ccc1ccccc21 ; CC1(C)c2cc(-c3ccc(-c4nc(-c5ccccc5)nc(-c5ccc(-c6ccccc6)nc5)n4)cc3)ccc2-c2ccc3ccccc3c21 ; CC1(C)c2ccccc2-c2cc(-c3ccc(-c4nc(-c5ccccc5)nc(-c5ccc(-c6ccccn6)cc5)n4)cc3)ccc21]

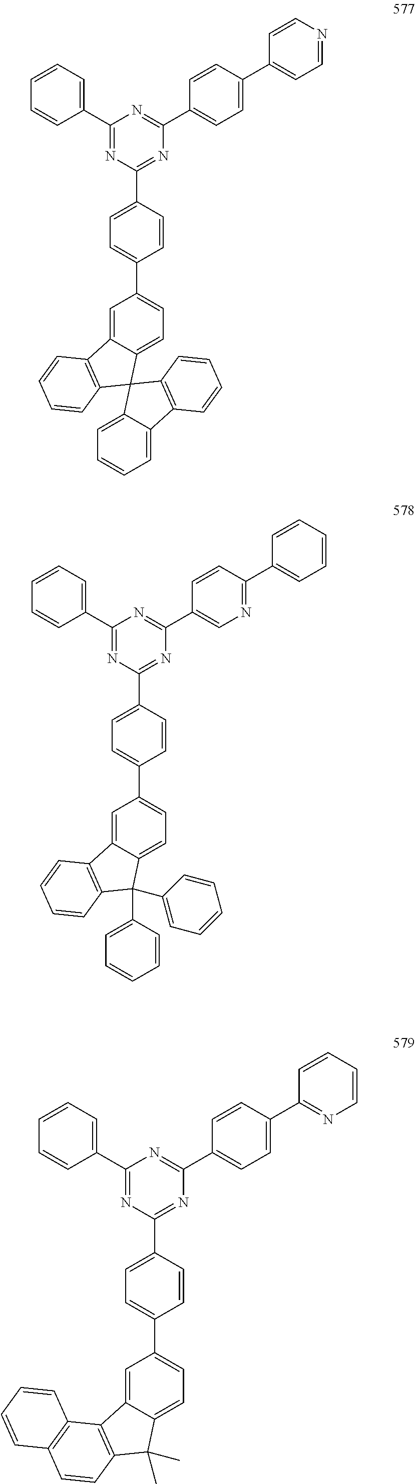 [SMILES: c1ccc(-c2nc(-c3ccc(-c4ccncc4)cc3)nc(-c3ccc(-c4ccc5c(c4)-c4ccccc4C54c5ccccc5-c5ccccc54)cc3)n2)cc1 ; c1ccc(-c2ccc(-c3nc(-c4ccccc4)nc(-c4ccc(-c5ccc6c(c5)-c5ccccc5C6(c5ccccc5)c5ccccc5)cc4)n3)cn2)cc1 ; CC1(C)c2ccc(-c3ccc(-c4nc(-c5ccccc5)nc(-c5ccc(-c6ccccn6)cc5)n4)cc3)cc2-c2c1ccc1ccccc21]

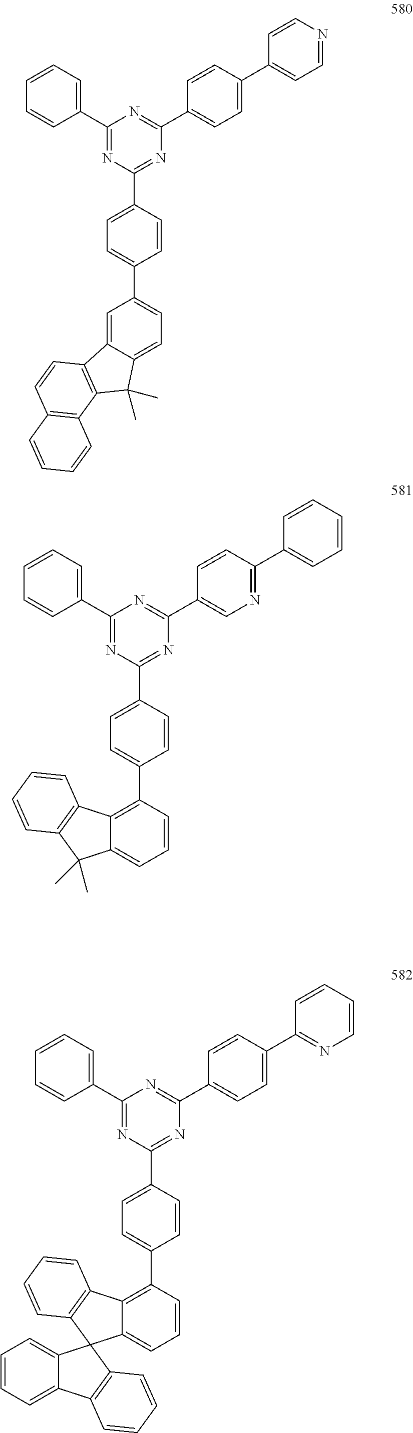 [SMILES: CC1(C)c2ccc(-c3ccc(-c4nc(-c5ccccc5)nc(-c5ccc(-c6ccncc6)cc5)n4)cc3)cc2-c2ccc3ccccc3c21 ; CC1(C)c2ccccc2-c2c(-c3ccc(-c4nc(-c5ccccc5)nc(-c5ccc(-c6ccccc6)nc5)n4)cc3)cccc21 ; c1ccc(-c2nc(-c3ccc(-c4ccccn4)cc3)nc(-c3ccc(-c4cccc5c4-c4ccccc4C54c5ccccc5-c5ccccc54)cc3)n2)cc1]

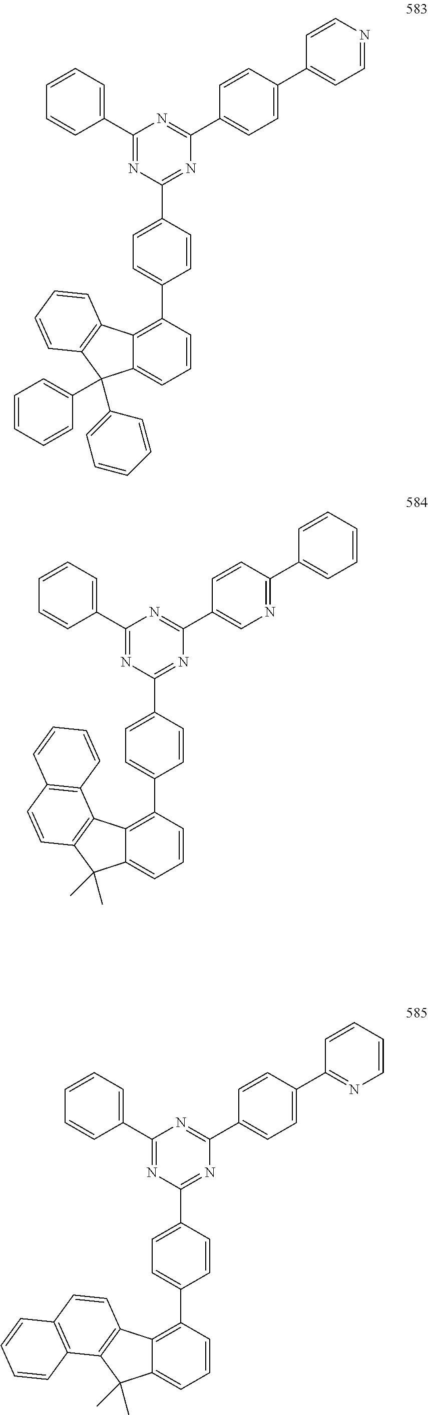 [SMILES: c1ccc(-c2nc(-c3ccc(-c4ccncc4)cc3)nc(-c3ccc(-c4cccc5c4-c4ccccc4C5(c4ccccc4)c4ccccc4)cc3)n2)cc1 ; CC1(C)c2cccc(-c3ccc(-c4nc(-c5ccccc5)nc(-c5ccc(-c6ccccc6)nc5)n4)cc3)c2-c2c1ccc1ccccc21 ; CC1(C)c2cccc(-c3ccc(-c4nc(-c5ccccc5)nc(-c5ccc(-c6ccccn6)cc5)n4)cc3)c2-c2ccc3ccccc3c21]

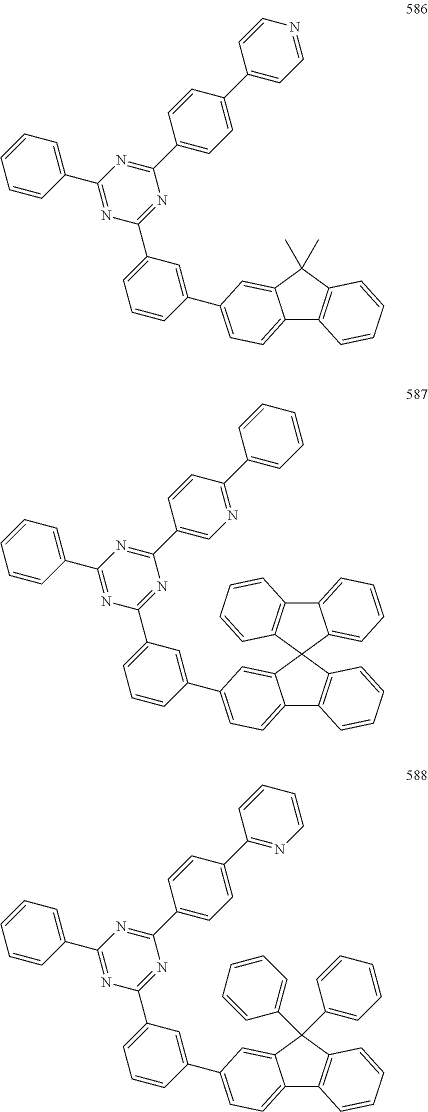 [SMILES: CC1(C)c2ccccc2-c2ccc(-c3cccc(-c4nc(-c5ccccc5)nc(-c5ccc(-c6ccncc6)cc5)n4)c3)cc21 ; c1ccc(-c2ccc(-c3nc(-c4ccccc4)nc(-c4cccc(-c5ccc6c(c5)C5(c7ccccc7-c7ccccc75)c5ccccc5-6)c4)n3)cn2)cc1 ; c1ccc(-c2nc(-c3ccc(-c4ccccn4)cc3)nc(-c3cccc(-c4ccc5c(c4)C(c4ccccc4)(c4ccccc4)c4ccccc4-5)c3)n2)cc1]

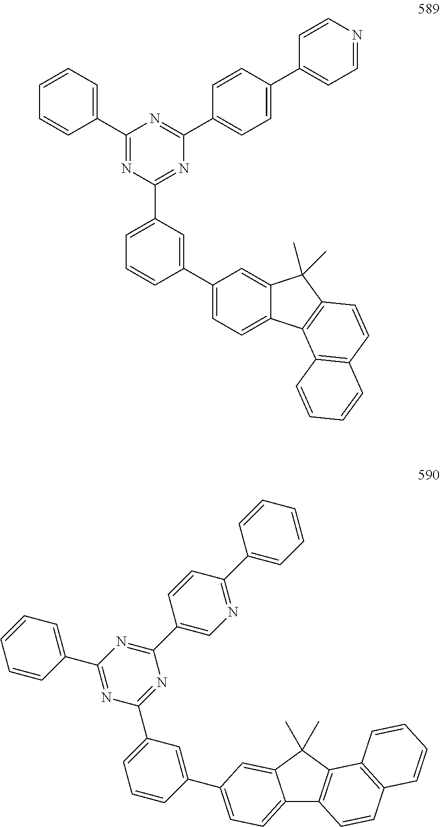 [SMILES: CC1(C)c2cc(-c3cccc(-c4nc(-c5ccccc5)nc(-c5ccc(-c6ccncc6)cc5)n4)c3)ccc2-c2c1ccc1ccccc21 ; CC1(C)c2cc(-c3cccc(-c4nc(-c5ccccc5)nc(-c5ccc(-c6ccccc6)nc5)n4)c3)ccc2-c2ccc3ccccc3c21]

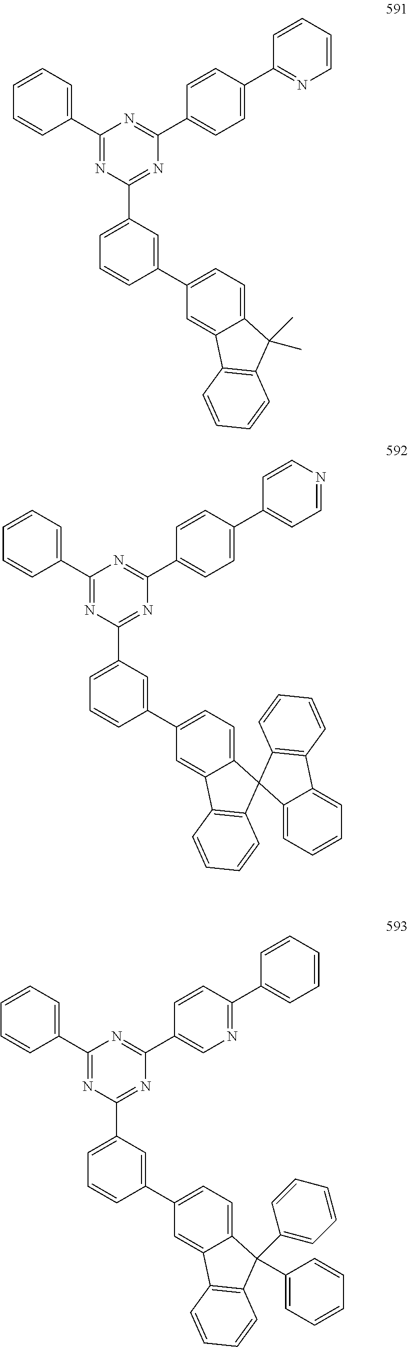 [SMILES: CC1(C)c2ccccc2-c2cc(-c3cccc(-c4nc(-c5ccccc5)nc(-c5ccc(-c6ccccn6)cc5)n4)c3)ccc21 ; c1ccc(-c2nc(-c3ccc(-c4ccncc4)cc3)nc(-c3cccc(-c4ccc5c(c4)-c4ccccc4C54c5ccccc5-c5ccccc54)c3)n2)cc1 ; c1ccc(-c2ccc(-c3nc(-c4ccccc4)nc(-c4cccc(-c5ccc6c(c5)-c5ccccc5C6(c5ccccc5)c5ccccc5)c4)n3)cn2)cc1]

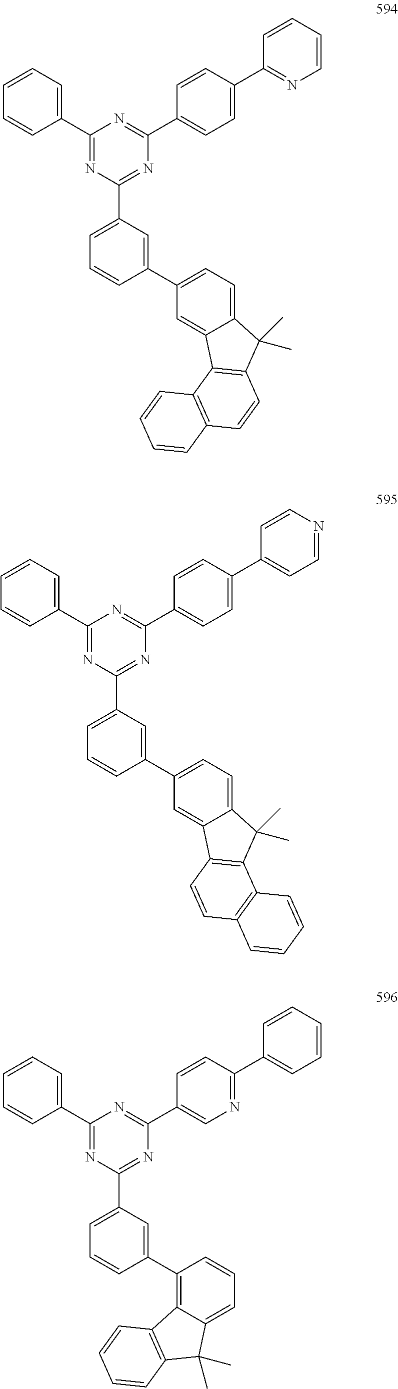 [SMILES: CC1(C)c2ccc(-c3cccc(-c4nc(-c5ccccc5)nc(-c5ccc(-c6ccccn6)cc5)n4)c3)cc2-c2c1ccc1ccccc21 ; CC1(C)c2ccc(-c3cccc(-c4nc(-c5ccccc5)nc(-c5ccc(-c6ccncc6)cc5)n4)c3)cc2-c2ccc3ccccc3c21 ; CC1(C)c2ccccc2-c2c(-c3cccc(-c4nc(-c5ccccc5)nc(-c5ccc(-c6ccccc6)nc5)n4)c3)cccc21]

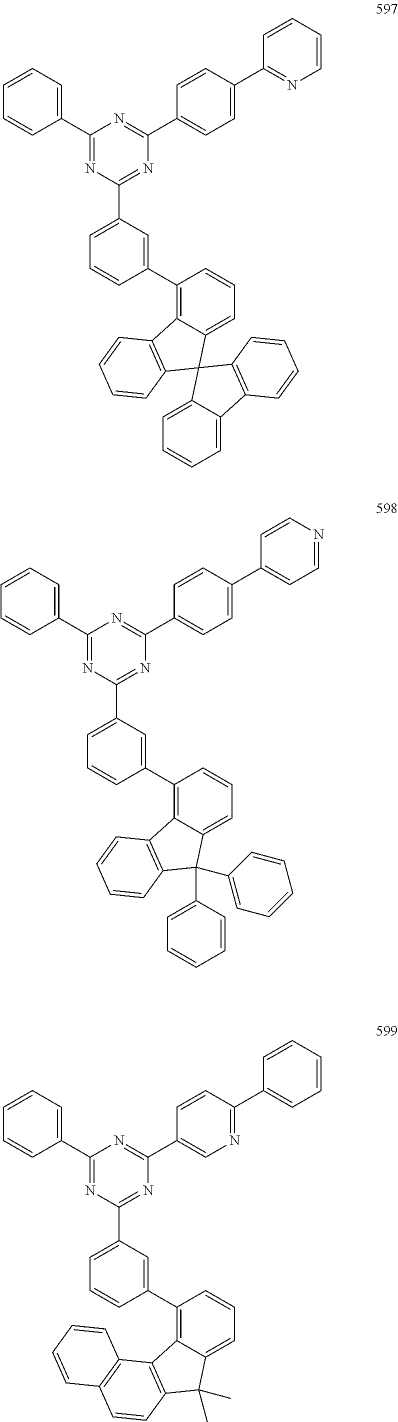 [SMILES: c1ccc(-c2nc(-c3ccc(-c4ccccn4)cc3)nc(-c3cccc(-c4cccc5c4-c4ccccc4C54c5ccccc5-c5ccccc54)c3)n2)cc1 ; c1ccc(-c2nc(-c3ccc(-c4ccncc4)cc3)nc(-c3cccc(-c4cccc5c4-c4ccccc4C5(c4ccccc4)c4ccccc4)c3)n2)cc1 ; CC1(C)c2cccc(-c3cccc(-c4nc(-c5ccccc5)nc(-c5ccc(-c6ccccc6)nc5)n4)c3)c2-c2c1ccc1ccccc21]

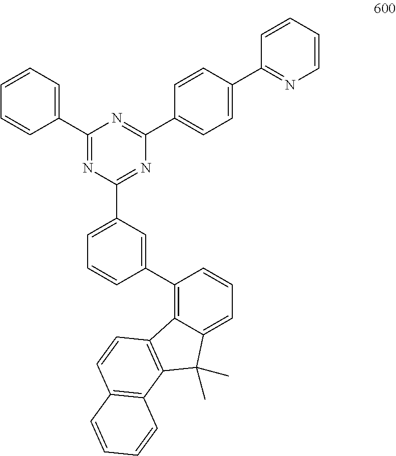 [SMILES: CC1(C)c2cccc(-c3cccc(-c4nc(-c5ccccc5)nc(-c5ccc(-c6ccccn6)cc5)n4)c3)c2-c2ccc3ccccc3c21]

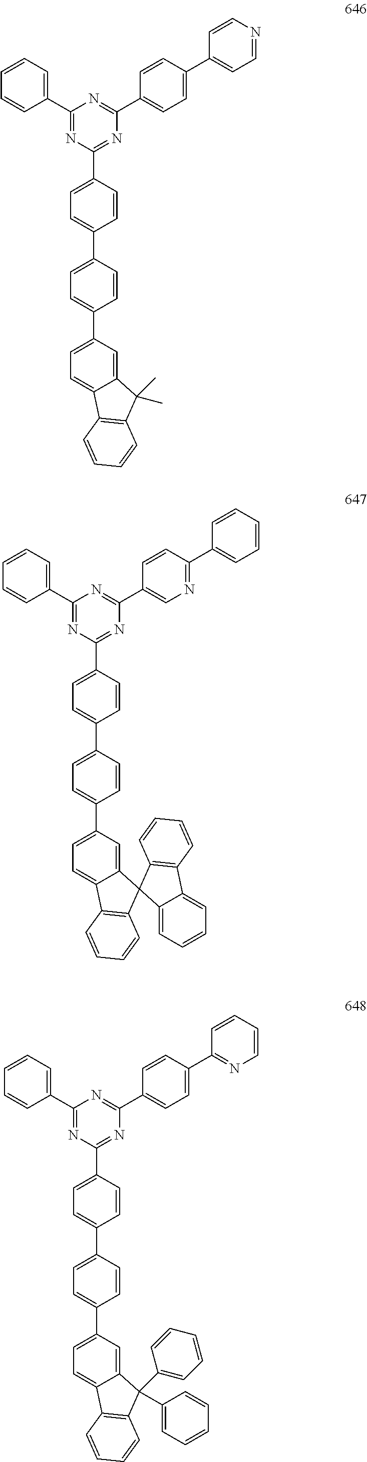 [SMILES: CC1(C)c2ccccc2-c2ccc(-c3ccc(-c4ccc(-c5nc(-c6ccccc6)nc(-c6ccc(-c7ccncc7)cc6)n5)cc4)cc3)cc21 ; c1ccc(-c2ccc(-c3nc(-c4ccccc4)nc(-c4ccc(-c5ccc(-c6ccc7c(c6)C6(c8ccccc8-c8ccccc86)c6ccccc6-7)cc5)cc4)n3)cn2)cc1 ; c1ccc(-c2nc(-c3ccc(-c4ccc(-c5ccc6c(c5)C(c5ccccc5)(c5ccccc5)c5ccccc5-6)cc4)cc3)nc(-c3ccc(-c4ccccn4)cc3)n2)cc1]

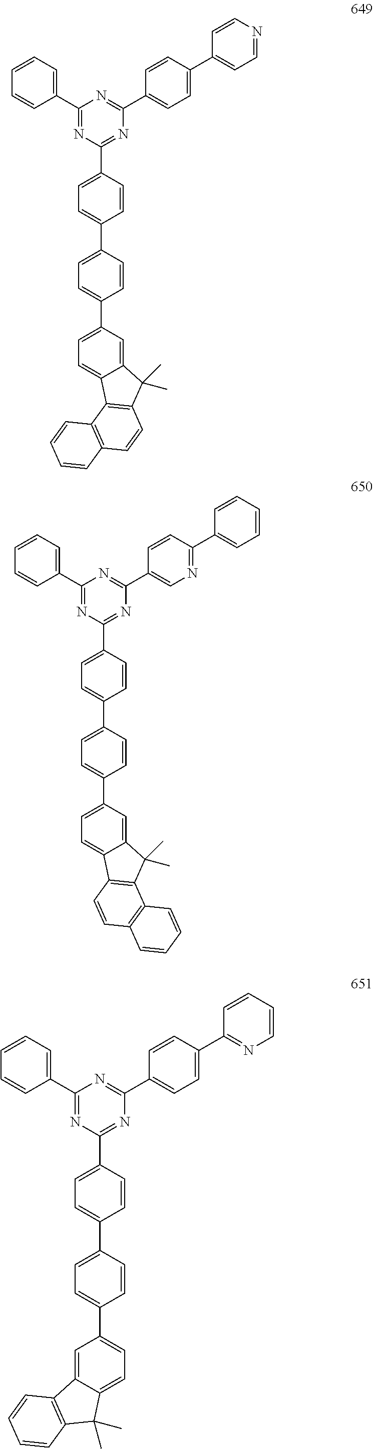 [SMILES: CC1(C)c2cc(-c3ccc(-c4ccc(-c5nc(-c6ccccc6)nc(-c6ccc(-c7ccncc7)cc6)n5)cc4)cc3)ccc2-c2c1ccc1ccccc21 ; CC1(C)c2cc(-c3ccc(-c4ccc(-c5nc(-c6ccccc6)nc(-c6ccc(-c7ccccc7)nc6)n5)cc4)cc3)ccc2-c2ccc3ccccc3c21 ; CC1(C)c2ccccc2-c2cc(-c3ccc(-c4ccc(-c5nc(-c6ccccc6)nc(-c6ccc(-c7ccccn7)cc6)n5)cc4)cc3)ccc21]

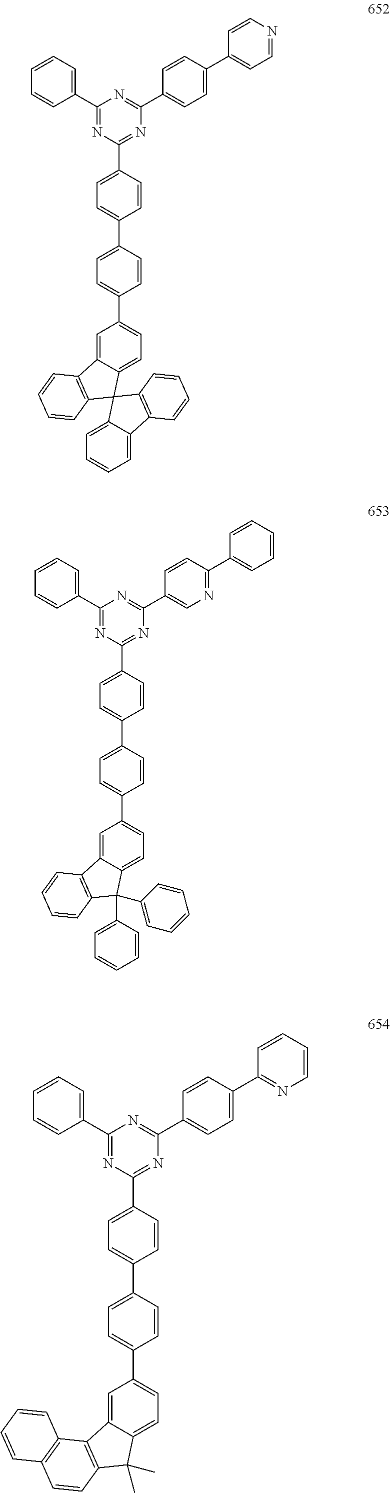 [SMILES: c1ccc(-c2nc(-c3ccc(-c4ccncc4)cc3)nc(-c3ccc(-c4ccc(-c5ccc6c(c5)-c5ccccc5C65c6ccccc6-c6ccccc65)cc4)cc3)n2)cc1 ; c1ccc(-c2ccc(-c3nc(-c4ccccc4)nc(-c4ccc(-c5ccc(-c6ccc7c(c6)-c6ccccc6C7(c6ccccc6)c6ccccc6)cc5)cc4)n3)cn2)cc1 ; CC1(C)c2ccc(-c3ccc(-c4ccc(-c5nc(-c6ccccc6)nc(-c6ccc(-c7ccccn7)cc6)n5)cc4)cc3)cc2-c2c1ccc1ccccc21]

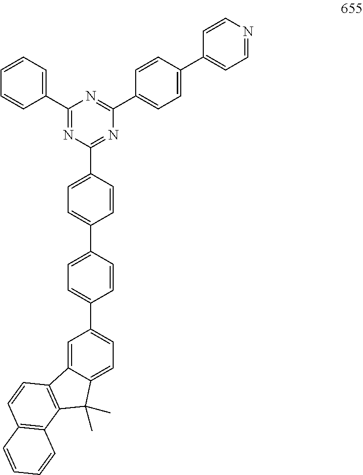 [SMILES: CC1(C)c2ccc(-c3ccc(-c4ccc(-c5nc(-c6ccccc6)nc(-c6ccc(-c7ccncc7)cc6)n5)cc4)cc3)cc2-c2ccc3ccccc3c21]

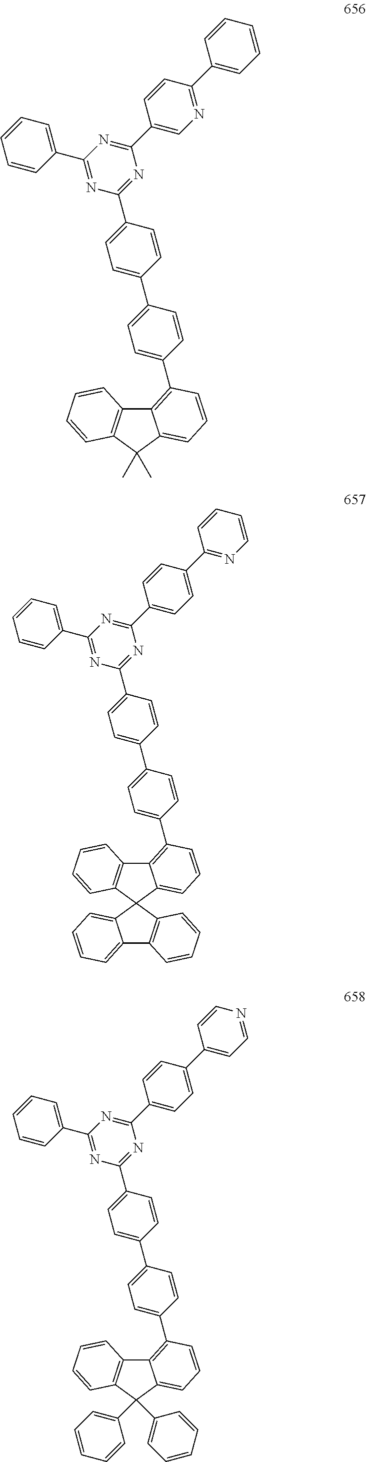 [SMILES: CC1(C)c2ccccc2-c2c(-c3ccc(-c4ccc(-c5nc(-c6ccccc6)nc(-c6ccc(-c7ccccc7)nc6)n5)cc4)cc3)cccc21 ; c1ccc(-c2nc(-c3ccc(-c4ccc(-c5cccc6c5-c5ccccc5C65c6ccccc6-c6ccccc65)cc4)cc3)nc(-c3ccc(-c4ccccn4)cc3)n2)cc1 ; c1ccc(-c2nc(-c3ccc(-c4ccncc4)cc3)nc(-c3ccc(-c4ccc(-c5cccc6c5-c5ccccc5C6(c5ccccc5)c5ccccc5)cc4)cc3)n2)cc1]

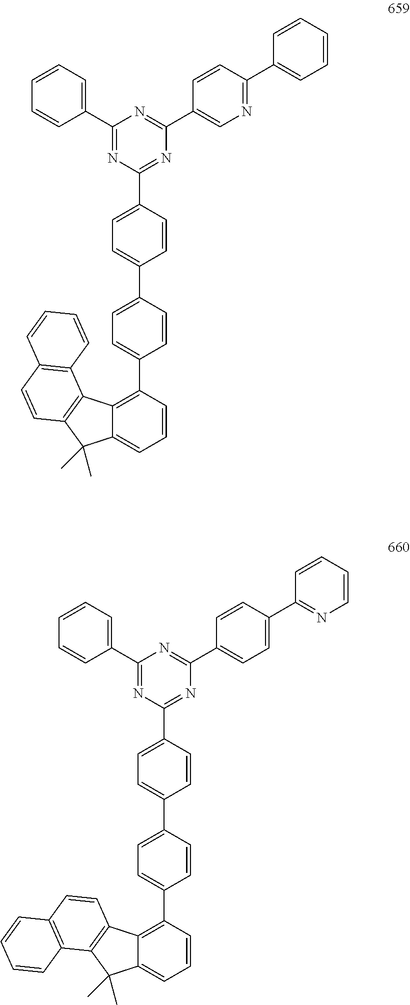 [SMILES: CC1(C)c2cccc(-c3ccc(-c4ccc(-c5nc(-c6ccccc6)nc(-c6ccc(-c7ccccc7)nc6)n5)cc4)cc3)c2-c2c1ccc1ccccc21 ; CC1(C)c2cccc(-c3ccc(-c4ccc(-c5nc(-c6ccccc6)nc(-c6ccc(-c7ccccn7)cc6)n5)cc4)cc3)c2-c2ccc3ccccc3c21]

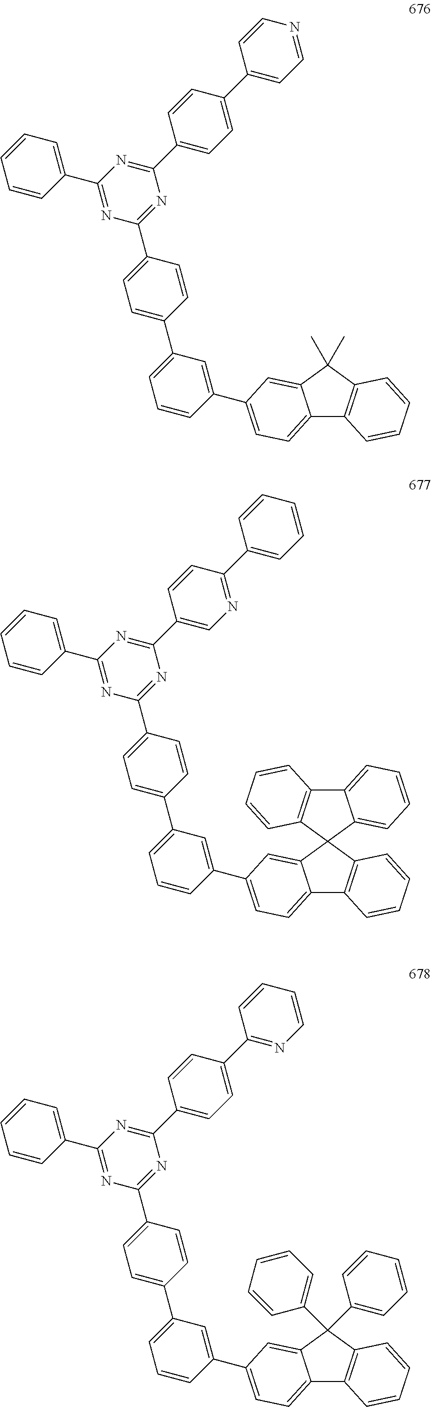 [SMILES: CC1(C)c2ccccc2-c2ccc(-c3cccc(-c4ccc(-c5nc(-c6ccccc6)nc(-c6ccc(-c7ccncc7)cc6)n5)cc4)c3)cc21 ; c1ccc(-c2ccc(-c3nc(-c4ccccc4)nc(-c4ccc(-c5cccc(-c6ccc7c(c6)C6(c8ccccc8-c8ccccc86)c6ccccc6-7)c5)cc4)n3)cn2)cc1 ; c1ccc(-c2nc(-c3ccc(-c4cccc(-c5ccc6c(c5)C(c5ccccc5)(c5ccccc5)c5ccccc5-6)c4)cc3)nc(-c3ccc(-c4ccccn4)cc3)n2)cc1]

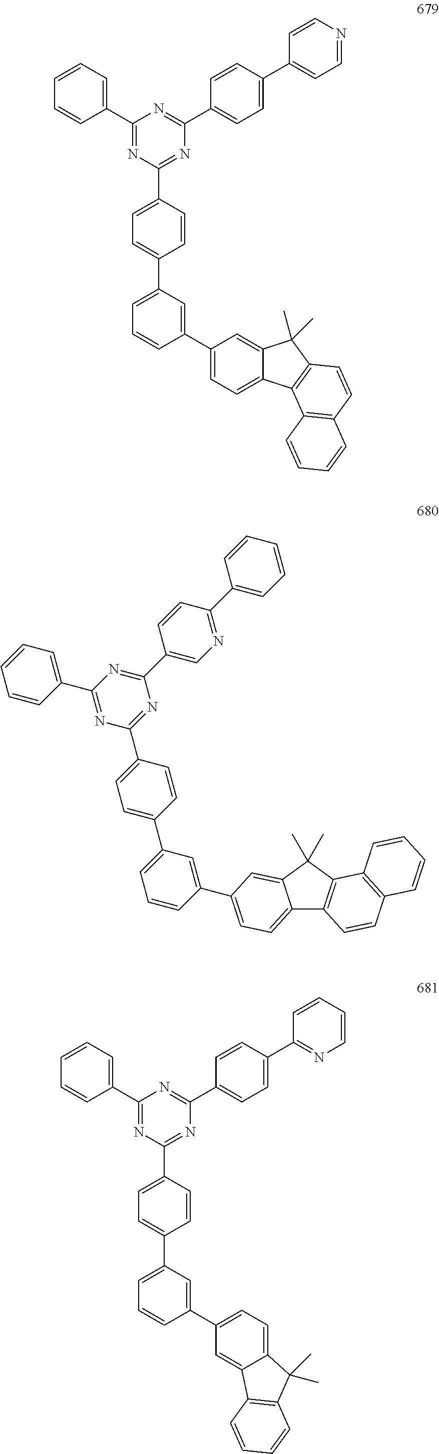 [SMILES: CC1(C)c2cc(-c3cccc(-c4ccc(-c5nc(-c6ccccc6)nc(-c6ccc(-c7ccncc7)cc6)n5)cc4)c3)ccc2-c2c1ccc1ccccc21 ; CC1(C)c2cc(-c3cccc(-c4ccc(-c5nc(-c6ccccc6)nc(-c6ccc(-c7ccccc7)nc6)n5)cc4)c3)ccc2-c2ccc3ccccc3c21 ; CC1(C)c2ccccc2-c2cc(-c3cccc(-c4ccc(-c5nc(-c6ccccc6)nc(-c6ccc(-c7ccccn7)cc6)n5)cc4)c3)ccc21]

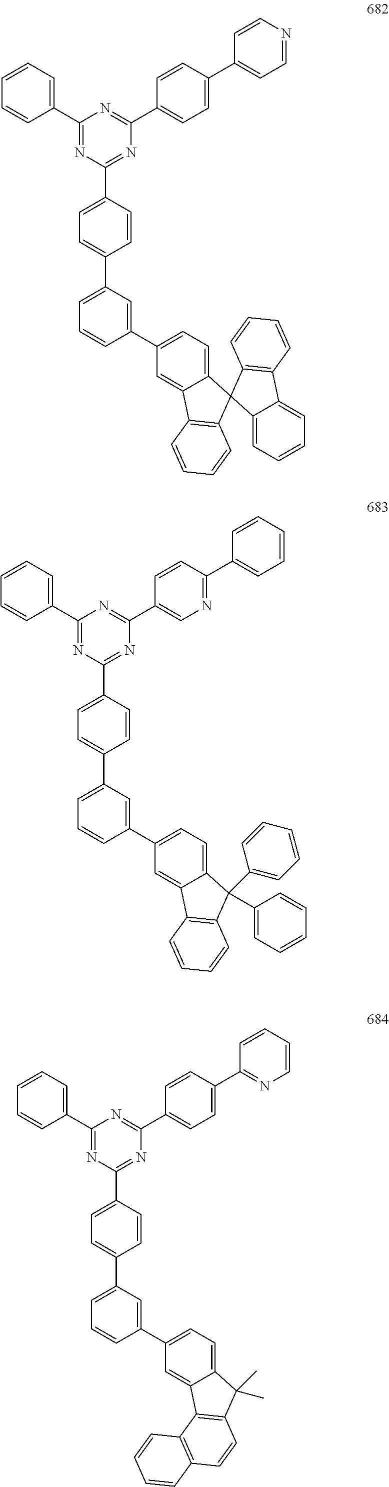 [SMILES: c1ccc(-c2nc(-c3ccc(-c4ccncc4)cc3)nc(-c3ccc(-c4cccc(-c5ccc6c(c5)-c5ccccc5C65c6ccccc6-c6ccccc65)c4)cc3)n2)cc1 ; c1ccc(-c2ccc(-c3nc(-c4ccccc4)nc(-c4ccc(-c5cccc(-c6ccc7c(c6)-c6ccccc6C7(c6ccccc6)c6ccccc6)c5)cc4)n3)cn2)cc1 ; CC1(C)c2ccc(-c3cccc(-c4ccc(-c5nc(-c6ccccc6)nc(-c6ccc(-c7ccccn7)cc6)n5)cc4)c3)cc2-c2c1ccc1ccccc21]

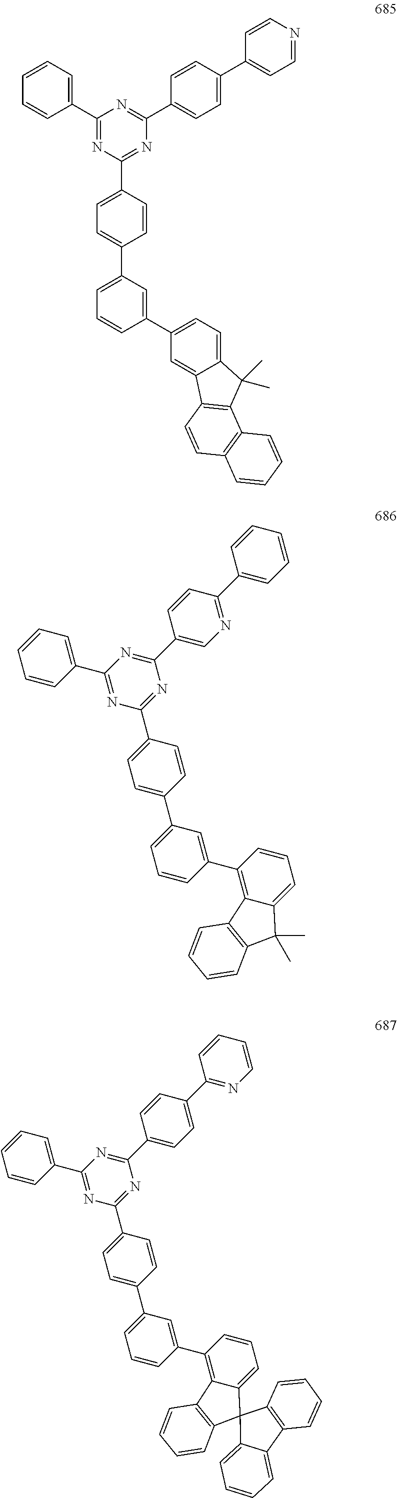 [SMILES: CC1(C)c2ccc(-c3cccc(-c4ccc(-c5nc(-c6ccccc6)nc(-c6ccc(-c7ccncc7)cc6)n5)cc4)c3)cc2-c2ccc3ccccc3c21 ; CC1(C)c2ccccc2-c2c(-c3cccc(-c4ccc(-c5nc(-c6ccccc6)nc(-c6ccc(-c7ccccc7)nc6)n5)cc4)c3)cccc21 ; c1ccc(-c2nc(-c3ccc(-c4cccc(-c5cccc6c5-c5ccccc5C65c6ccccc6-c6ccccc65)c4)cc3)nc(-c3ccc(-c4ccccn4)cc3)n2)cc1]

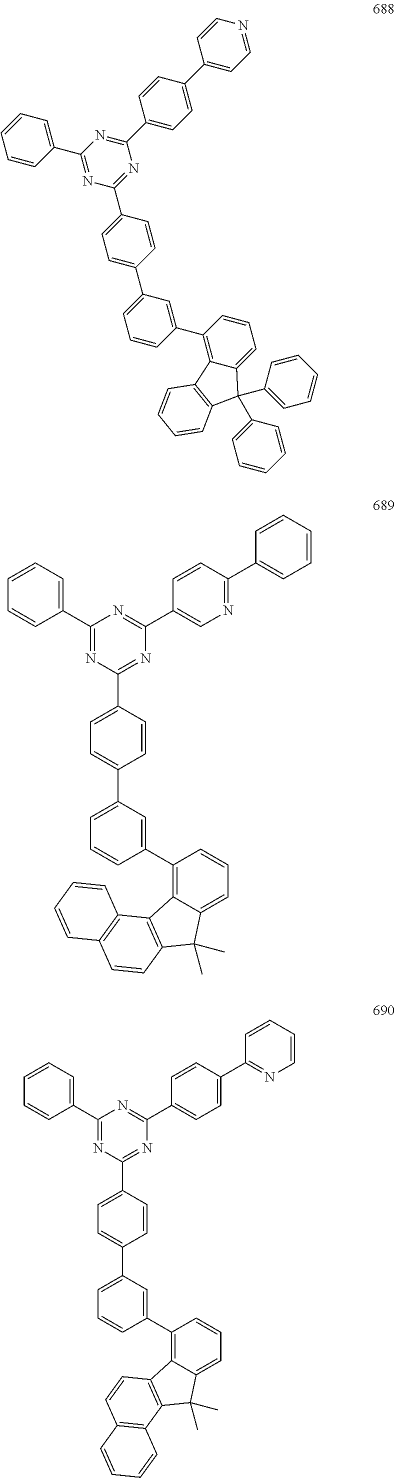 [SMILES: c1ccc(-c2nc(-c3ccc(-c4ccncc4)cc3)nc(-c3ccc(-c4cccc(-c5cccc6c5-c5ccccc5C6(c5ccccc5)c5ccccc5)c4)cc3)n2)cc1 ; CC1(C)c2cccc(-c3cccc(-c4ccc(-c5nc(-c6ccccc6)nc(-c6ccc(-c7ccccc7)nc6)n5)cc4)c3)c2-c2c1ccc1ccccc21 ; CC1(C)c2cccc(-c3cccc(-c4ccc(-c5nc(-c6ccccc6)nc(-c6ccc(-c7ccccn7)cc6)n5)cc4)c3)c2-c2ccc3ccccc3c21]

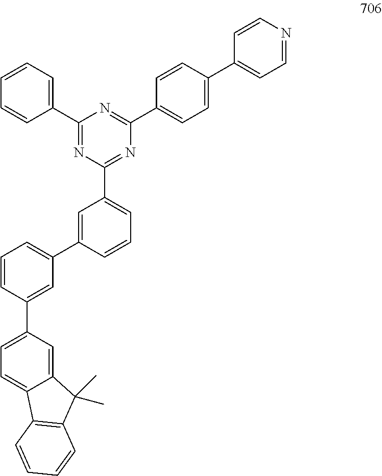 [SMILES: CC1(C)c2ccccc2-c2ccc(-c3cccc(-c4cccc(-c5nc(-c6ccccc6)nc(-c6ccc(-c7ccncc7)cc6)n5)c4)c3)cc21]

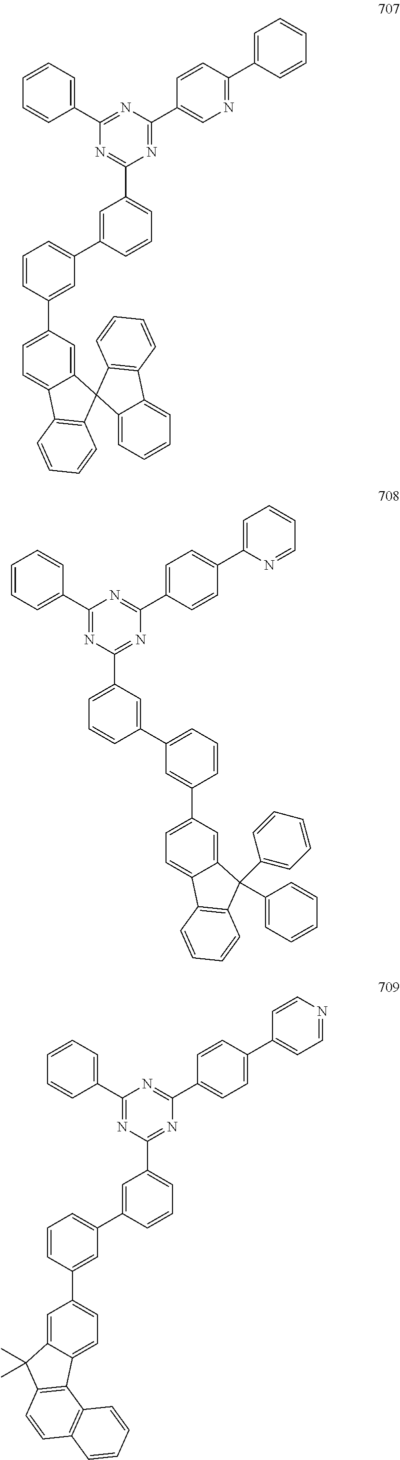 [SMILES: c1ccc(-c2ccc(-c3nc(-c4ccccc4)nc(-c4cccc(-c5cccc(-c6ccc7c(c6)C6(c8ccccc8-c8ccccc86)c6ccccc6-7)c5)c4)n3)cn2)cc1 ; c1ccc(-c2nc(-c3ccc(-c4ccccn4)cc3)nc(-c3cccc(-c4cccc(-c5ccc6c(c5)C(c5ccccc5)(c5ccccc5)c5ccccc5-6)c4)c3)n2)cc1 ; CC1(C)c2cc(-c3cccc(-c4cccc(-c5nc(-c6ccccc6)nc(-c6ccc(-c7ccncc7)cc6)n5)c4)c3)ccc2-c2c1ccc1ccccc21]

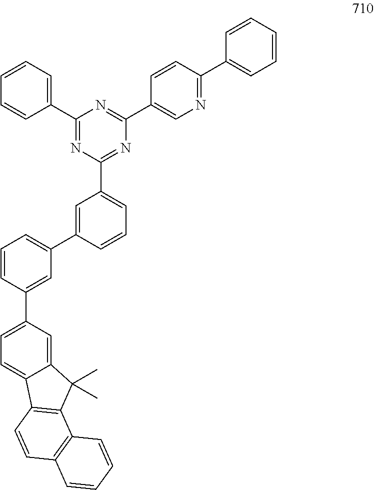 [SMILES: CC1(C)c2cc(-c3cccc(-c4cccc(-c5nc(-c6ccccc6)nc(-c6ccc(-c7ccccc7)nc6)n5)c4)c3)ccc2-c2ccc3ccccc3c21]

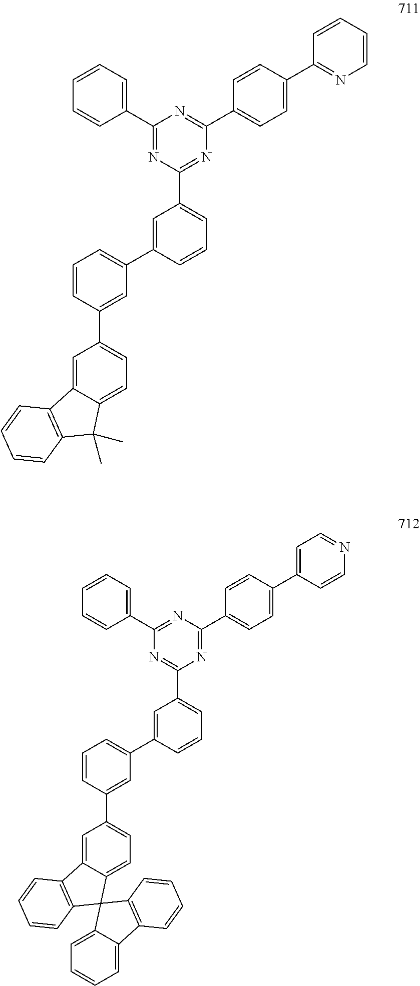 [SMILES: CC1(C)c2ccccc2-c2cc(-c3cccc(-c4cccc(-c5nc(-c6ccccc6)nc(-c6ccc(-c7ccccn7)cc6)n5)c4)c3)ccc21 ; c1ccc(-c2nc(-c3ccc(-c4ccncc4)cc3)nc(-c3cccc(-c4cccc(-c5ccc6c(c5)-c5ccccc5C65c6ccccc6-c6ccccc65)c4)c3)n2)cc1]

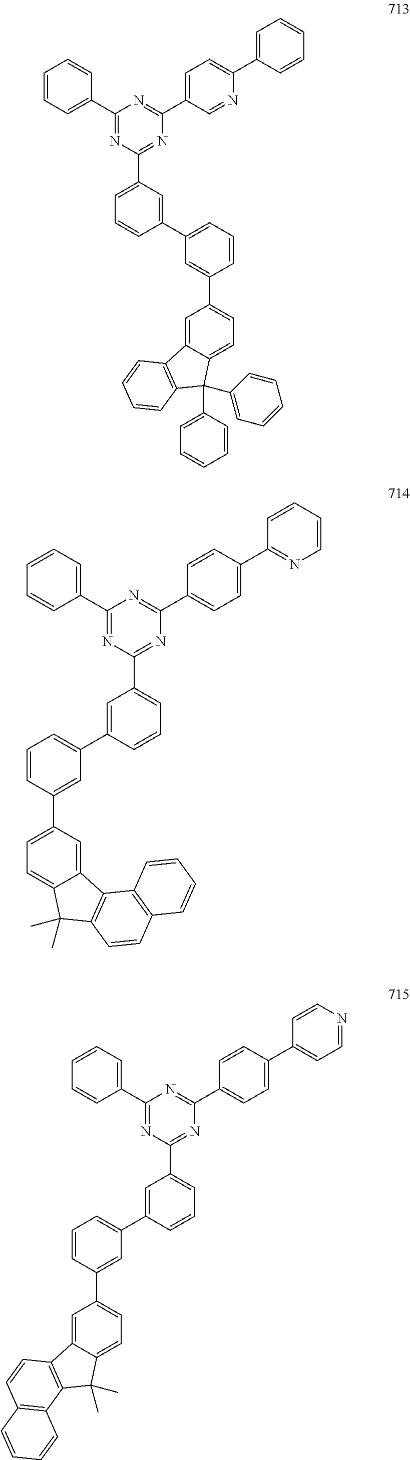 [SMILES: c1ccc(-c2ccc(-c3nc(-c4ccccc4)nc(-c4cccc(-c5cccc(-c6ccc7c(c6)-c6ccccc6C7(c6ccccc6)c6ccccc6)c5)c4)n3)cn2)cc1 ; CC1(C)c2ccc(-c3cccc(-c4cccc(-c5nc(-c6ccccc6)nc(-c6ccc(-c7ccccn7)cc6)n5)c4)c3)cc2-c2c1ccc1ccccc21 ; CC1(C)c2ccc(-c3cccc(-c4cccc(-c5nc(-c6ccccc6)nc(-c6ccc(-c7ccncc7)cc6)n5)c4)c3)cc2-c2ccc3ccccc3c21]

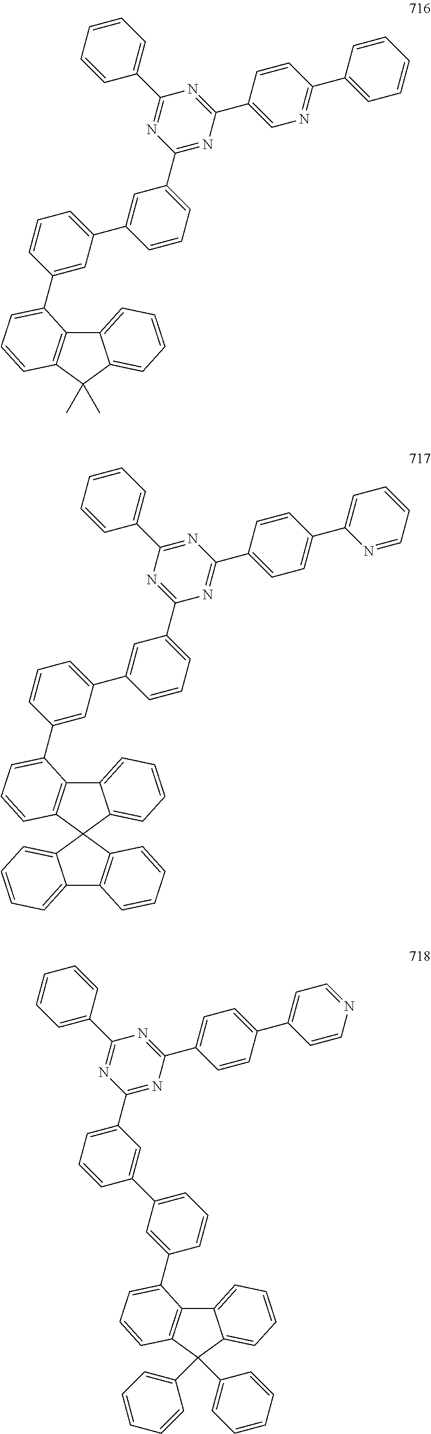 [SMILES: CC1(C)c2ccccc2-c2c(-c3cccc(-c4cccc(-c5nc(-c6ccccc6)nc(-c6ccc(-c7ccccc7)nc6)n5)c4)c3)cccc21 ; c1ccc(-c2nc(-c3ccc(-c4ccccn4)cc3)nc(-c3cccc(-c4cccc(-c5cccc6c5-c5ccccc5C65c6ccccc6-c6ccccc65)c4)c3)n2)cc1 ; c1ccc(-c2nc(-c3ccc(-c4ccncc4)cc3)nc(-c3cccc(-c4cccc(-c5cccc6c5-c5ccccc5C6(c5ccccc5)c5ccccc5)c4)c3)n2)cc1]

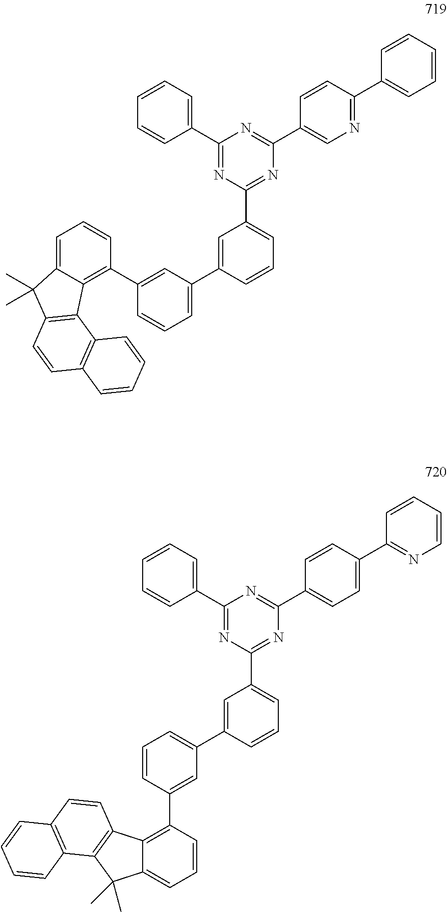 [SMILES: CC1(C)c2cccc(-c3cccc(-c4cccc(-c5nc(-c6ccccc6)nc(-c6ccc(-c7ccccc7)nc6)n5)c4)c3)c2-c2c1ccc1ccccc21 ; CC1(C)c2cccc(-c3cccc(-c4cccc(-c5nc(-c6ccccc6)nc(-c6ccc(-c7ccccn7)cc6)n5)c4)c3)c2-c2ccc3ccccc3c21]

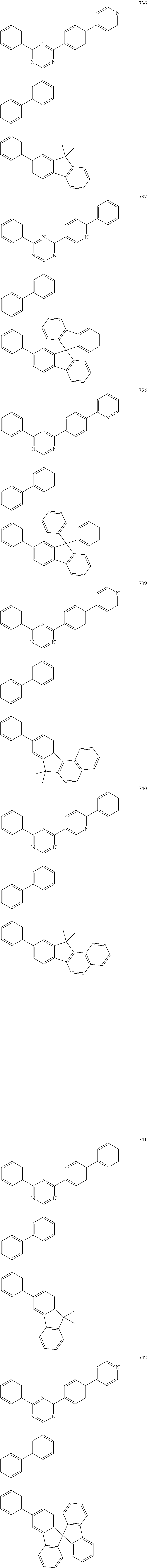

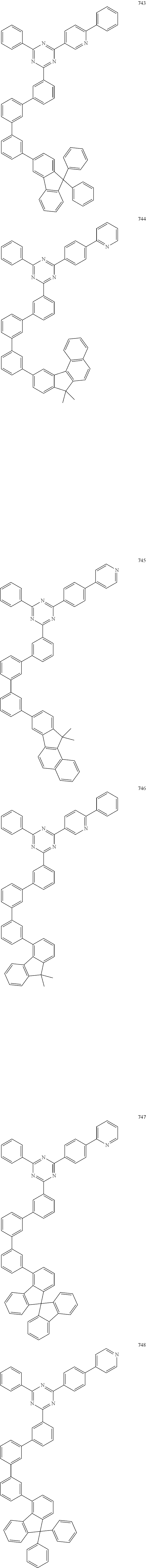

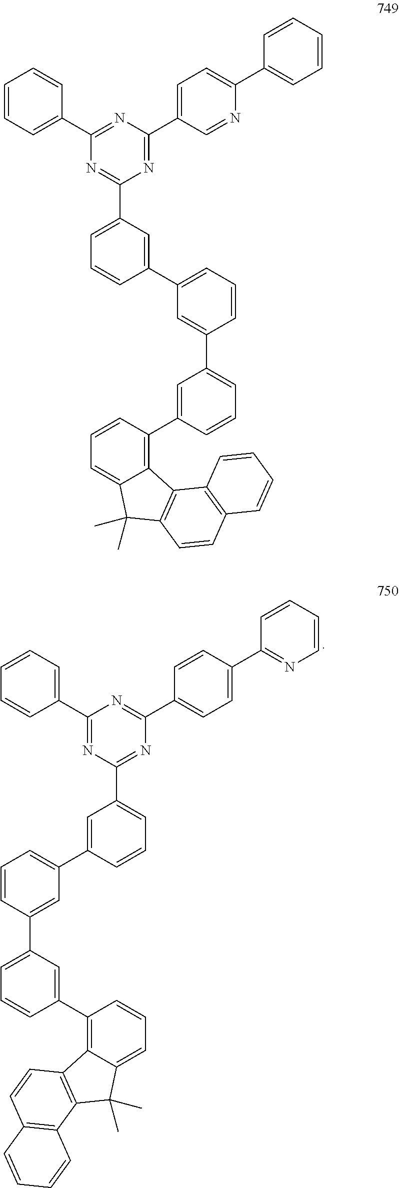 [SMILES: CC1(C)c2cccc(-c3cccc(-c4cccc(-c5cccc(-c6nc(-c7ccccc7)nc(-c7ccc(-c8ccccc8)nc7)n6)c5)c4)c3)c2-c2c1ccc1ccccc21 ; CC1(C)c2cccc(-c3cccc(-c4cccc(-c5cccc(-c6nc(-c7ccccc7)nc(-c7ccc(-c8ccccn8)cc7)n6)c5)c4)c3)c2-c2ccc3ccccc3c21]

17. The organic electroluminescent device of claim 9, wherein the organic layer comprising the compound is selected from the group consisting of: a hole injection layer, a hole transporting layer, a light emitting auxiliary layer, a light emitting layer, an electron transporting layer, and an electron injection layer.

18. The organic electroluminescent device of claim 9, wherein the organic layer comprising the compound is selected from the group consisting of: an electron transporting layer and an electron transport auxiliary layer.
